SEQUENCE LISTING (1219 sequences)

SEQ ID NO: 1 - DNA, 41 bp, Homo sapiens
tattgttttttgtttgtttgttttcaagtttgggactaaaa

SEQ ID NO: 2 - DNA, 41 bp, Homo sapiens
cctgcagagggagcgtcaaggccctgtgctgctgtccctgg

SEQ ID NO: 3 - DNA, 41 bp, Homo sapiens
ggcgggaggattgcttgagcccaggactttgagaccagcct

SEQ ID NO: 4 - DNA, 41 bp, Homo sapiens
gcacaaaaagaatagagatggaagactagggctaaggtagc

SEQ ID NO: 5 - DNA, 41 bp, Homo sapiens
agacttttccaatgaagagcgttttacggggtttgctgcat

SEQ ID NO: 6 - DNA, 41 bp, Homo sapiens
tcttacccttatggttttggatttaacctcattctcccttc

SEQ ID NO: 7 - DNA, 41 bp, Homo sapiens
gtcccttgtagccacttctgcaacaatttcattggtggtta

SEQ ID NO: 8 - DNA, 41 bp, Homo sapiens
ggattccctgggcctctaaatattgaaaccaagagtaatgc

SEQ ID NO: 9 - DNA, 41 bp, Homo sapiens
caattctgtttgggagcctgcgaaggcaaaatatgtcttta

SEQ ID NO: 10 - DNA, 41 bp, Homo sapiens
agcctgcgaaggcaaaatatgtctttagagatgtggtgcag

SEQ ID NO: 11 - DNA, 41 bp, Homo sapiens
ccacagagaggctggtgtggggaggttcatcccaggtgtgc

SEQ ID NO: 12 - DNA, 41 bp, Homo sapiens
ttggggagagatgatagccatgggtgactgggagtcacctt

SEQ ID NO: 13 - DNA, 41 bp, Homo sapiens
acctcttccgactacaacctcatggatggggacctgggact

SEQ ID NO: 14 - DNA, 41 bp, Homo sapiens
tcccagactccacggtgctccgcatgcacccagcttccccc

SEQ ID NO: 15 - DNA, 41 bp, Homo sapiens
ggccctggagctgggcttccctgagatcagccccagggcac

SEQ ID NO: 16 - DNA, 41 bp, Homo sapiens
cagcctgggtcatcaggaactagactggctcacaggcagag

SEQ ID NO: 17 - DNA, 41 bp, Homo sapiens
atggaggccctgcaccagccgctgggatggacacatgtggg

SEQ ID NO: 18 - DNA, 41 bp, Homo sapiens
ctggctactcggcttcctctgtaaatgaggaaggcctggcg

SEQ ID NO: 19 - DNA, 41 bp, Homo sapiens
tgtgctccctcagggccacgtgggagaagaatggagtgccc

SEQ ID NO: 20 - DNA, 41 bp, Homo sapiens
aatggagtgcccctttggcctgggaaggaagtaggcagagc

SEQ ID NO: 21 - DNA, 41 bp, Homo sapiens
cccctttggcctgggaaggaagtaggcagagcccaggtctg

SEQ ID NO: 22 - DNA, 41 bp, Homo sapiens
tcacacctactgggtagggagccatgattatagaggagatg

SEQ ID NO: 23 - DNA, 41 bp, Homo sapiens
ccccccggggccccagcctcggcgctcgctgtccctgacac

SEQ ID NO: 24 - DNA, 41 bp, Homo sapiens
tcccagaccagaccttgcccaatgacgttgttggtaatgct

SEQ ID NO: 25 - DNA, 41 bp, Homo sapiens
cggattaagtccgggttcagggtgtagctcaggtacctaag

SEQ ID NO: 26 - DNA, 41 bp, Homo sapiens
ctaagagggccagatgctgtctcagctactgctaattcagg

SEQ ID NO: 27 - DNA, 41 bp, Homo sapiens
ctctcgcagtcccaccctgcgccaagactcacctgttcagg

SEQ ID NO: 28 - DNA, 41 bp, Homo sapiens
tttcggaactgctcccaggcgaagtcccactcctcctcccc

SEQ ID NO: 29 - DNA, 41 bp, Homo sapiens
cctccccgccctgggcgatagcgttgcagtagacggtggac

SEQ ID NO: 30 - DNA, 41 bp, Homo sapiens
gcgatagcgttgcagtagacggtggaccgcaggttggggtg

SEQ ID NO: 31 - DNA, 41 bp, Homo sapiens
tagttctggggaaaagaaaatatgaatctcatcaaagactc

SEQ ID NO: 32 - DNA, 41 bp, Homo sapiens
gtggggacccagggagggacgtccagggagcacaggagctc

SEQ ID NO: 33 - DNA, 41 bp, Homo sapiens
gagctcagggcacagcacgtggcatatgggaagggcagcag

SEQ ID NO: 34 - DNA, 41 bp, Homo sapiens
gaagtcacgagcttctgcagctgagccaggcagcggagcac

SEQ ID NO: 35 - DNA, 41 bp, Homo sapiens
tgttgaatggaatggcccatcacagccctcagaacatgggc

SEQ ID NO: 36 - DNA, 41 bp, Homo sapiens
gctggattacctcttacatctatcagccagtagtcctgctg

SEQ ID NO: 37 - DNA, 41 bp, Homo sapiens
ctggattacctcttacatctatcagccagtagtcctgctgc

SEQ ID NO: 38 - DNA, 41 bp, Homo sapiens
ccctccaggccaagtccccacctccttccccgtgccccacg

SEQ ID NO: 39 - DNA, 41 bp, Homo sapiens
acctccttccccgtgccccacgaggagcgggctgcaccttg

SEQ ID NO: 40 - DNA, 41 bp, Homo sapiens
catgccccccgcaccagacccctgggcagctggcttaccaa

SEQ ID NO: 41 - DNA, 41 bp, Homo sapiens
ctggaggaggacagggggtcgaacagcagggagttctcccg

SEQ ID NO: 42 - DNA, 41 bp, Homo sapiens
tctggtctggggaggcgatgccattggcaggatgaactccg

SEQ ID NO: 43 - DNA, 41 bp, Homo sapiens
aagaagttaaggatggggcccgtcacgttcagggcataatc

SEQ ID NO: 44 - DNA, 41 bp, Homo sapiens
aggatggggcccgtcacgttcagggcataatcgccgtggcc

SEQ ID NO: 45 - DNA, 41 bp, Homo sapiens
gggcataatcgccgtggcccgccgcaatggcactgggccgg

SEQ ID NO: 46 - DNA, 41 bp, Homo sapiens
caggtgggctgggtcccagggcccagtgggctggggggatc

SEQ ID NO: 47 - DNA, 41 bp, Homo sapiens
cctggccctgtggccgcaggcagggcccactcacctttggg

SEQ ID NO: 48 - DNA, 41 bp, Homo sapiens
tctgcagcctgcatctgtgtagtggccaccaccctgcccca

SEQ ID NO: 49 - DNA, 41 bp, Homo sapiens
agcacctcggatccaccccaccgggcagcccagccggaact

SEQ ID NO: 50 - DNA, 41 bp, Homo sapiens
ttgctgtggatgatgatgacgtcagtggcctccttgcaggt

SEQ ID NO: 51 - DNA, 41 bp, Homo sapiens
ggctccaacaggcgaaggtcactggactgggcaggggcacg

SEQ ID NO: 52 - DNA, 41 bp, Homo sapiens
agttcctccaggttcccctccctgccgcttcttgccaaata

SEQ ID NO: 53 - DNA, 41 bp, Homo sapiens
taaaatgaaatgagttgttttgctttttttgctgaaggctt

SEQ ID NO: 54 - DNA, 41 bp, Homo sapiens
caggtttgttgaacagatttagtgagaaaacatattaaaca

SEQ ID NO: 55 - DNA, 41 bp, Homo sapiens
gtgagaaaacatattaaacaccaaatagtagaatgattaaa

SEQ ID NO: 56 - DNA, 41 bp, Homo sapiens
aggccgaggtgggtggatcacgaggttaggagttcaagacc

SEQ ID NO: 57 - DNA, 41 bp, Homo sapiens
agttgggcgtggtggcgggcgtctgtaatcccagctacttg

SEQ ID NO: 58 - DNA, 41 bp, Homo sapiens
ggcagagaattgcttgaatccgggaggcagagattgcagtg

SEQ ID NO: 59 - DNA, 41 bp, Homo sapiens
attgcagtgagctgagatcgcaccactgcactccagcctgg

SEQ ID NO: 60 - DNA, 41 bp, Homo sapiens
ttgcagtgagctgagatcgcaccactgcactccagcctggg

SEQ ID NO: 61 - DNA, 41 bp, Homo sapiens
gtggtgtgatctcggctcaccgcaacctctgcctcccaggt

SEQ ID NO: 62 - DNA, 41 bp, Homo sapiens
agctgggattacagacatgcaccaccacacctggctaattt

SEQ ID NO: 63 - DNA, 41 bp, Homo sapiens
tctagtagagatggggtttcactgtgttggccaggttgggc

SEQ ID NO: 64 - DNA, 41 bp, Homo sapiens
gttggccaggttgggcttgaactcccgacctcaggtggtcc

SEQ ID NO: 65 - DNA, 41 bp, Homo sapiens
ataggcactcaataatttttattaaatgagtgaatgataaa

SEQ ID NO: 66 - DNA, 41 bp, Homo sapiens
ttgggactggatctttttgagggtgacatcagacttgatag

SEQ ID NO: 67 - DNA, 41 bp, Homo sapiens
ttcaaagggtgggcttatatagtctttaaggttttgtttgg

SEQ ID NO: 68 - DNA, 41 bp, Homo sapiens
gctggagaaacaaactatatatcagtgttcaagggcagtgg

SEQ ID NO: 69 - DNA, 41 bp, Homo sapiens
ctgccctgagacctcagattgtaacattctcttcccccttt

SEQ ID NO: 70 - DNA, 41 bp, Homo sapiens
acatttctctttttttcctatgtttttagttaaggactgaa

SEQ ID NO: 71 - DNA, 41 bp, Homo sapiens
tttagttaaggactgaatttctaagcatgtgtcctctcttg

SEQ ID NO: 72 - DNA, 41 bp, Homo sapiens
tccttgagttttatggactcgtgcagttttgaactggaaaa

SEQ ID NO: 73 - DNA, 41 bp, Homo sapiens
taatgattattcattaggttactactgagaagtaggccttg

SEQ ID NO: 74 - DNA, 41 bp, Homo sapiens
ctctcaattctgcttttctttaacaggttctggtttcttca

SEQ ID NO: 75 - DNA, 41 bp, Homo sapiens
taaatgtgggggccacagcagtgctggaaagtagaacgctg

SEQ ID NO: 76 - DNA, 41 bp, Homo sapiens
tgtttgaaggacgcaaaggctctggtgcatcactgggtggt

SEQ ID NO: 77 - DNA, 41 bp, Homo sapiens
attgatgacatcaatctttcggaaacacggtgccctcatca

SEQ ID NO: 78 - DNA, 41 bp, Homo sapiens
aaccagtgcctttataacccacgaaaggctgaaaagcagag

SEQ ID NO: 79 - DNA, 41 bp, Homo sapiens
accagtgcctttataacccacgaaaggctgaaaagcagaga

SEQ ID NO: 80 - DNA, 41 bp, Homo sapiens
tttatataaactagtttttttttgttgtggccactgattat

SEQ ID NO: 81 - DNA, 41 bp, Homo sapiens
ttccacttttagatgtattatatagagatgtggggggaatg

SEQ ID NO: 82 - DNA, 41 bp, Homo sapiens
atcaaatgattgttaaacaaaagctgatttccatccacact

SEQ ID NO: 83 - DNA, 41 bp, Homo sapiens
aagagaggctccacccgagacaccatagtcattgctgtttc

SEQ ID NO: 84 - DNA, 41 bp, Homo sapiens
aaatcgaccaaatttgactccgcaaaatgtaagttgaggct

SEQ ID NO: 85 - DNA, 41 bp, Homo sapiens
ggctgatgtttgattattcataacctattggtgaaatctta

SEQ ID NO: 86 - DNA, 41 bp, Homo sapiens
cttcatttaaagaccagatcattttattatgattcatttaa

SEQ ID NO: 87 - DNA, 41 bp, Homo sapiens
aaagtggatatttttctgtaaatagctggaaatattataaa

SEQ ID NO: 88 - DNA, 41 bp, Homo sapiens
ttcccaatcacatccctgcaggtcaggtggtaattgttgag

SEQ ID NO: 89 - DNA, 41 bp, Homo sapiens
cacaagctgagagaaaaggtgtgcaacaccctttctcctta

SEQ ID NO: 90 - DNA, 41 bp, Homo sapiens
attaaaaaatacaaaacaacgaatagctttcaaaaaggctc

SEQ ID NO: 91 - DNA, 41 bp, Homo sapiens
aagaaggtgacctgaaagacataaagagccacttaattgta

SEQ ID NO: 92 - DNA, 41 bp, Homo sapiens
tgcagaatggggacagatgtctaatcctgtagttttctggt

SEQ ID NO: 93 - DNA, 41 bp, Homo sapiens
aggagcaattttactaccccacatgtaatttttacttcttt

SEQ ID NO: 94 - DNA, 41 bp, Homo sapiens
gagcaattttactaccccacatgtaatttttacttctttat

SEQ ID NO: 95 - DNA, 41 bp, Homo sapiens
tatcaaacagtcttttgtgagaacagagtcacaactgggct

SEQ ID NO: 96 - DNA, 41 bp, Homo sapiens
accagctgagtgctctcagctggagctcagctgtccacctt

SEQ ID NO: 97 - DNA, 41 bp, Homo sapiens
agcagcaaggccaggcagcatgctcctccgcatgccttacg

SEQ ID NO: 98 - DNA, 41 bp, Homo sapiens
ctgcctgttgcctgtaacagaaagacagaaagcagagtggc

SEQ ID NO: 99 - DNA, 41 bp, Homo sapiens
cacgaggctcctgggtccccccaaatggatccttacctgcg

SEQ ID NO: 100 - DNA, 41 bp, Homo sapiens
aaagctcactttttcctcaattgcttcatcccaactgacca

SEQ ID NO: 101 - DNA, 41 bp, Homo sapiens
gcatccactctattatggaacttttcttcattcttgttact

SEQ ID NO: 102 - DNA, 41 bp, Homo sapiens
ggctgaggctcagagagattctaaccttgtgccaggctcaa

SEQ ID NO: 103 - DNA, 41 bp, Homo sapiens
cggtggtcctagaggcaaagggcagtagggtgggaaatcaa

SEQ ID NO: 104 - DNA, 41 bp, Homo sapiens
gagcctgcagatggaggagaggtgggtggcagaaaaaggag

SEQ ID NO: 105 - DNA, 41 bp, Homo sapiens
aatatcaggcagtgatgggtgggccaggaccagaaagggca

SEQ ID NO: 106 - DNA, 41 bp, Homo sapiens
cagccctggtcccagaaagcagtaggaacccagtgaaagaa

SEQ ID NO: 107 - DNA, 41 bp, Homo sapiens
tcgacttggattcggaggacgaggctggagacaatagcaag

SEQ ID NO: 108 - DNA, 41 bp, Homo sapiens
cttggattcggaggacgaggctggagacaatagcaagaggt

SEQ ID NO: 109 - DNA, 41 bp, Homo sapiens
acgcccattgcagcagggcagggagcagggccttgaaagtc

SEQ ID NO: 110 - DNA, 41 bp, Homo sapiens
gcccattgcagcagggcagggagcagggccttgaaagtcca

SEQ ID NO: 111 - DNA, 41 bp, Homo sapiens
gcctggagaaagtagcgcccgtcagtccacatggctgcatg

SEQ ID NO: 112 - DNA, 41 bp, Homo sapiens
gactgttctgcctgcttagccttgtgctaggctcagcgggg

SEQ ID NO: 113 - DNA, 41 bp, Homo sapiens
atttcaaaaccagcctgcccccagcaattcaacgtccagtt

SEQ ID NO: 114 - DNA, 41 bp, Homo sapiens
taggtgtttatctttgtttatagtagaaaaaagcaggagaa

SEQ ID NO: 115 - DNA, 41 bp, Homo sapiens
aaagcaggagaagtgtatggtcagttaaataaactataata

SEQ ID NO: 116 - DNA, 41 bp, Homo sapiens
agatgacaggctgggcgcagtggctcacgcctttaattcca

SEQ ID NO: 117 - DNA, 41 bp, Homo sapiens
agtttctagaaggacagtcacccaagtgttaagagtggtta

SEQ ID NO: 118 - DNA, 41 bp, Homo sapiens
agctctcagaagccagttcagaggctgagtcccctccctca

SEQ ID NO: 119 - DNA, 41 bp, Homo sapiens
gctctcagaagccagttcagaggctgagtcccctccctcag

SEQ ID NO: 120 - DNA, 41 bp, Homo sapiens
aagtctgtcaccttctggacgtgggcttttttgcactcatc

SEQ ID NO: 121 - DNA, 41 bp, Homo sapiens
ctgcttccctggccctttctcccttggacgcaggagtcccc

SEQ ID NO: 122 - DNA, 41 bp, Homo sapiens
ctttagcattacaaaatccagtgtccctcccaaacaatgct

SEQ ID NO: 123 - DNA, 41 bp, Homo sapiens
aatgagcctcccacttcttgctcccagttatgggtgctcaa

SEQ ID NO: 124 - DNA, 41 bp, Homo sapiens
ggtctctgaagataattaggaactagattcctgcacctcaa

SEQ ID NO: 125 - DNA, 41 bp, Homo sapiens
gcattcctctctgggaaatcaccttcctcgaacccaaagag

SEQ ID NO: 126 - DNA, 41 bp, Homo sapiens
tgggaatcagcttgctggagggaaggggaccgaattaagga

SEQ ID NO: 127 - DNA, 41 bp, Homo sapiens
accctctgtctgctcgagcccaggaaaggcctgaaggaaga

SEQ ID NO: 128 - DNA, 41 bp, Homo sapiens
gaggccggggaaagagccctccctctctcccttgtccctcc

SEQ ID NO: 129 - DNA, 41 bp, Homo sapiens
gcttcctggaagaggcacttggtgtgcttgggcttgtagct

SEQ ID NO: 130 - DNA, 41 bp, Homo sapiens
ctgcaagtttctctgttctgccccaggaactgcagtggtga

SEQ ID NO: 131 - DNA, 41 bp, Homo sapiens
tataaagaaatggaccttggtagaaggaggggcggtgggac

SEQ ID NO: 132 - DNA, 41 bp, Homo sapiens
agggaaccaggactaactttgcctgaatcacaattttttcc

SEQ ID NO: 133 - DNA, 41 bp, Homo sapiens
gatacccaaggtgggtttgccaggccccagcccaagccagg

SEQ ID NO: 134 - DNA, 41 bp, Homo sapiens
cagaggcgccagctactagaggagttcgagtggcttcaaca

SEQ ID NO: 135 - DNA, 41 bp, Homo sapiens
tccaagacctatggagaaggtcccaggccccaggaacacag

SEQ ID NO: 136 - DNA, 41 bp, Homo sapiens
attggccgttagccacctgggtccacatcctgctaagacgt

SEQ ID NO: 137 - DNA, 41 bp, Homo sapiens
aaacccattgctctgatggccttgaagatgatggaactttc

SEQ ID NO: 138 - DNA, 41 bp, Homo sapiens
ggcctcctccttcctgtcttggcctttgttttgggctgcta

SEQ ID NO: 139 - DNA, 41 bp, Homo sapiens
gagacatcttcaatttcttctttgttggagtggtctgcata

SEQ ID NO: 140 - DNA, 41 bp, Homo sapiens
acatcttcaatttcttctttgttggagtggtctgcatagga

SEQ ID NO: 141 - DNA, 41 bp, Homo sapiens
aagaagaaaacaaaattagcataaggagaaatttttgaaag

SEQ ID NO: 142 - DNA, 41 bp, Homo sapiens
tttttgaaagcatctgcgaatgcaactcatatacacacgca

SEQ ID NO: 143 - DNA, 41 bp, Homo sapiens
tccctggcctacttgattcatgtgctctgtattttcttctt

SEQ ID NO: 144 - DNA, 41 bp, Homo sapiens
tttcagaactaaccaataaatagattccatgttttcttgtt

SEQ ID NO: 145 - DNA, 41 bp, Homo sapiens
caaagagccctgaggatgagggacgtgggatcattccccgg

SEQ ID NO: 146 - DNA, 41 bp, Homo sapiens
atcattccccggtttcccccgggccaggtgctaggtcgttt

SEQ ID NO: 147 - DNA, 41 bp, Homo sapiens
aggcggctataggtggcaagcggagctgtccagcactagtg

SEQ ID NO: 148 - DNA, 41 bp, Homo sapiens
tattgttttttgtttgtttgctttcaagtttgggactaaaa

SEQ ID NO: 149 - DNA, 41 bp, Homo sapiens
cctgcagagggagcgtcaagaccctgtgctgctgtccctgg

SEQ ID NO: 150 - DNA, 41 bp, Homo sapiens
ggcgggaggattgcttgagctcaggactttgagaccagcct

SEQ ID NO: 151 - DNA, 41 bp, Homo sapiens
gcacaaaaagaatagagatgaaagactagggctaaggtagc

SEQ ID NO: 152 - DNA, 41 bp, Homo sapiens
agacttttccaatgaagagcattttacggggtttgctgcat

SEQ ID NO: 153 - DNA, 41 bp, Homo sapiens
tcttacccttatggttttgggtttaacctcattctcccttc

SEQ ID NO: 154 - DNA, 41 bp, Homo sapiens
gtcccttgtagccacttctgtaacaatttcattggtggtta

SEQ ID NO: 155 - DNA, 41 bp, Homo sapiens
ggattccctgggcctctaaaaattgaaaccaagagtaatgc

SEQ ID NO: 156 - DNA, 41 bp, Homo sapiens
caattctgtttgggagcctgtgaaggcaaaatatgtcttta

SEQ ID NO: 157 - DNA, 41 bp, Homo sapiens
agcctgcgaaggcaaaatatatctttagagatgtggtgcag

SEQ ID NO: 158 - DNA, 41 bp, Homo sapiens
ccacagagaggctggtgtggagaggttcatcccaggtgtgc

SEQ ID NO: 159 - DNA, 41 bp, Homo sapiens
ttggggagagatgatagccacgggtgactgggagtcacctt

SEQ ID NO: 160 - DNA, 41 bp, Homo sapiens
acctcttccgactacaaccttatggatggggacctgggact

SEQ ID NO: 161 - DNA, 41 bp, Homo sapiens
tcccagactccacggtgctctgcatgcacccagcttccccc

SEQ ID NO: 162 - DNA, 41 bp, Homo sapiens
ggccctggagctgggcttccttgagatcagccccagggcac

SEQ ID NO: 163 - DNA, 41 bp, Homo sapiens
cagcctgggtcatcaggaaccagactggctcacaggcagag

SEQ ID NO: 164 - DNA, 41 bp, Homo sapiens
atggaggccctgcaccagccactgggatggacacatgtggg

SEQ ID NO: 165 - DNA, 41 bp, Homo sapiens
ctggctactcggcttcctctataaatgaggaaggcctggcg

SEQ ID NO: 166 - DNA, 41 bp, Homo sapiens
tgtgctccctcagggccacgcgggagaagaatggagtgccc

SEQ ID NO: 167 - DNA, 41 bp, Homo sapiens
aatggagtgcccctttggccggggaaggaagtaggcagagc

SEQ ID NO: 168 - DNA, 41 bp, Homo sapiens
cccctttggcctgggaaggatgtaggcagagcccaggtctg

SEQ ID NO: 169 - DNA, 41 bp, Homo sapiens
tcacacctactgggtagggaaccatgattatagaggagatg

SEQ ID NO: 170 - DNA, 41 bp, Homo sapiens
ccccccggggccccagcctcagcgctcgctgtccctgacac

SEQ ID NO: 171 - DNA, 41 bp, Homo sapiens
tcccagaccagaccttgcccgatgacgttgttggtaatgct

SEQ ID NO: 172 - DNA, 41 bp, Homo sapiens
cggattaagtccgggttcagtgtgtagctcaggtacctaag

SEQ ID NO: 173 - DNA, 41 bp, Homo sapiens
ctaagagggccagatgctgtgtcagctactgctaattcagg

SEQ ID NO: 174 - DNA, 41 bp, Homo sapiens
ctctcgcagtcccaccctgccccaagactcacctgttcagg

SEQ ID NO: 175 - DNA, 41 bp, Homo sapiens
tttcggaactgctcccaggcaaagtcccactcctcctcccc

SEQ ID NO: 176 - DNA, 41 bp, Homo sapiens
cctccccgccctgggcgataacgttgcagtagacggtggac

SEQ ID NO: 177 - DNA, 41 bp, Homo sapiens
gcgatagcgttgcagtagacagtggaccgcaggttggggtg

SEQ ID NO: 178 - DNA, 41 bp, Homo sapiens
tagttctggggaaaagaaaacatgaatctcatcaaagactc

SEQ ID NO: 179 - DNA, 41 bp, Homo sapiens
gtggggacccagggagggacatccagggagcacaggagctc

SEQ ID NO: 180 - DNA, 41 bp, Homo sapiens
gagctcagggcacagcacgtagcatatgggaagggcagcag

SEQ ID NO: 181 - DNA, 41 bp, Homo sapiens
gaagtcacgagcttctgcagttgagccaggcagcggagcac

SEQ ID NO: 182 - DNA, 41 bp, Homo sapiens
tgttgaatggaatggcccatgacagccctcagaacatgggc

SEQ ID NO: 183 - DNA, 41 bp, Homo sapiens
gctggattacctcttacatccatcagccagtagtcctgctg

SEQ ID NO: 184 - DNA, 41 bp, Homo sapiens
ctggattacctcttacatctttcagccagtagtcctgctgc

SEQ ID NO: 185 - DNA, 41 bp, Homo sapiens
ccctccaggccaagtccccatctccttccccgtgccccacg

SEQ ID NO: 186 - DNA, 41 bp, Homo sapiens
acctccttccccgtgccccatgaggagcgggctgcaccttg

SEQ ID NO: 187 - DNA, 41 bp, Homo sapiens
catgccccccgcaccagacctctgggcagctggcttaccaa

SEQ ID NO: 188 - DNA, 41 bp, Homo sapiens
ctggaggaggacagggggtcaaacagcagggagttctcccg

SEQ ID NO: 189 - DNA, 41 bp, Homo sapiens
tctggtctggggaggcgatgtcattggcaggatgaactccg

SEQ ID NO: 190 - DNA, 41 bp, Homo sapiens
aagaagttaaggatggggcctgtcacgttcagggcataatc

SEQ ID NO: 191 - DNA, 41 bp, Homo sapiens
aggatggggcccgtcacgttaagggcataatcgccgtggcc

SEQ ID NO: 192 - DNA, 41 bp, Homo sapiens
gggcataatcgccgtggcccaccgcaatggcactgggccgg

SEQ ID NO: 193 - DNA, 41 bp, Homo sapiens
caggtgggctgggtcccaggacccagtgggctggggggatc

SEQ ID NO: 194 - DNA, 41 bp, Homo sapiens
cctggccctgtggccgcaggaagggcccactcacctttggg

SEQ ID NO: 195 - DNA, 41 bp, Homo sapiens
tctgcagcctgcatctgtgttgtggccaccaccctgcccca

SEQ ID NO: 196 - DNA, 41 bp, Homo sapiens
agcacctcggatccaccccagcgggcagcccagccggaact

SEQ ID NO: 197 - DNA, 41 bp, Homo sapiens
ttgctgtggatgatgatgacatcagtggcctccttgcaggt

SEQ ID NO: 198 - DNA, 41 bp, Homo sapiens
ggctccaacaggcgaaggtcgctggactgggcaggggcacg

SEQ ID NO: 199 - DNA, 41 bp, Homo sapiens
agttcctccaggttcccctcactgccgcttcttgccaaata

SEQ ID NO: 200 - DNA, 41 bp, Homo sapiens
taaaatgaaatgagttgtttcgctttttttgctgaaggctt

SEQ ID NO: 201 - DNA, 41 bp, Homo sapiens
caggtttgttgaacagatttcgtgagaaaacatattaaaca

SEQ ID NO: 202 - DNA, 41 bp, Homo sapiens
gtgagaaaacatattaaacagcaaatagtagaatgattaaa

SEQ ID NO: 203 - DNA, 41 bp, Homo sapiens
aggccgaggtgggtggatcatgaggttaggagttcaagacc

SEQ ID NO: 204 - DNA, 41 bp, Homo sapiens
agttgggcgtggtggcgggcatctgtaatcccagctacttg

SEQ ID NO: 205 - DNA, 41 bp, Homo sapiens
ggcagagaattgcttgaatctgggaggcagagattgcagtg

SEQ ID NO: 206 - DNA, 41 bp, Homo sapiens
attgcagtgagctgagatcgaaccactgcactccagcctgg

SEQ ID NO: 207 - DNA, 41 bp, Homo sapiens
ttgcagtgagctgagatcgccccactgcactccagcctggg

SEQ ID NO: 208 - DNA, 41 bp, Homo sapiens
gtggtgtgatctcggctcactgcaacctctgcctcccaggt

SEQ ID NO: 209 - DNA, 41 bp, Homo sapiens
agctgggattacagacatgccccaccacacctggctaattt

SEQ ID NO: 210 - DNA, 41 bp, Homo sapiens
tctagtagagatggggtttccctgtgttggccaggttgggc

SEQ ID NO: 211 - DNA, 41 bp, Homo sapiens
gttggccaggttgggcttgacctcccgacctcaggtggtcc

SEQ ID NO: 212 - DNA, 41 bp, Homo sapiens
ataggcactcaataatttttgttaaatgagtgaatgataaa

SEQ ID NO: 213 - DNA, 41 bp, Homo sapiens
ttgggactggatctttttgaaggtgacatcagacttgatag

SEQ ID NO: 214 - DNA, 41 bp, Homo sapiens
ttcaaagggtgggcttatatggtctttaaggttttgtttgg

SEQ ID NO: 215 - DNA, 41 bp, Homo sapiens
gctggagaaacaaactatatgtcagtgttcaagggcagtgg

SEQ ID NO: 216 - DNA, 41 bp, Homo sapiens
ctgccctgagacctcagattttaacattctcttcccccttt

SEQ ID NO: 217 - DNA, 41 bp, Homo sapiens
acatttctctttttttcctacgtttttagttaaggactgaa

SEQ ID NO: 218 - DNA, 41 bp, Homo sapiens
tttagttaaggactgaatttttaagcatgtgtcctctcttg

SEQ ID NO: 219 - DNA, 41 bp, Homo sapiens
tccttgagttttatggactcatgcagttttgaactggaaaa

SEQ ID NO: 220 - DNA, 41 bp, Homo sapiens
taatgattattcattaggttgctactgagaagtaggccttg

SEQ ID NO: 221 - DNA, 41 bp, Homo sapiens
ctctcaattctgcttttcttcaacaggttctggtttcttca

SEQ ID NO: 222 - DNA, 41 bp, Homo sapiens
taaatgtgggggccacagcaatgctggaaagtagaacgctg

SEQ ID NO: 223 - DNA, 41 bp, Homo sapiens
tgtttgaaggacgcaaaggcactggtgcatcactgggtggt

SEQ ID NO: 224 - DNA, 41 bp, Homo sapiens
attgatgacatcaatctttcagaaacacggtgccctcatca

SEQ ID NO: 225 - DNA, 41 bp, Homo sapiens
aaccagtgcctttataacccccgaaaggctgaaaagcagag

SEQ ID NO: 226 - DNA, 41 bp, Homo sapiens
accagtgcctttataacccatgaaaggctgaaaagcagaga

SEQ ID NO: 227 - DNA, 41 bp, Homo sapiens
tttatataaactagttttttattgttgtggccactgattat

SEQ ID NO: 228 - DNA, 41 bp, Homo sapiens
ttccacttttagatgtattagatagagatgtggggggaatg

SEQ ID NO: 229 - DNA, 41 bp, Homo sapiens
atcaaatgattgttaaacaacagctgatttccatccacact

SEQ ID NO: 230 - DNA, 41 bp, Homo sapiens
aagagaggctccacccgagataccatagtcattgctgtttc

SEQ ID NO: 231 - DNA, 41 bp, Homo sapiens
aaatcgaccaaatttgactctgcaaaatgtaagttgaggct

SEQ ID NO: 232 - DNA, 41 bp, Homo sapiens
ggctgatgtttgattattcacaacctattggtgaaatctta

SEQ ID NO: 233 - DNA, 41 bp, Homo sapiens
cttcatttaaagaccagatcgttttattatgattcatttaa

SEQ ID NO: 234 - DNA, 41 bp, Homo sapiens
aaagtggatatttttctgtacatagctggaaatattataaa

SEQ ID NO: 235 - DNA, 41 bp, Homo sapiens
ttcccaatcacatccctgcacgtcaggtggtaattgttgag

SEQ ID NO: 236 - DNA, 41 bp, Homo sapiens
cacaagctgagagaaaaggtatgcaacaccctttctcctta

SEQ ID NO: 237 - DNA, 41 bp, Homo sapiens
attaaaaaatacaaaacaactaatagctttcaaaaaggctc

SEQ ID NO: 238 - DNA, 41 bp, Homo sapiens
aagaaggtgacctgaaagacgtaaagagccacttaattgta

SEQ ID NO: 239 - DNA, 41 bp, Homo sapiens
tgcagaatggggacagatgtgtaatcctgtagttttctggt

SEQ ID NO: 240 - DNA, 41 bp, Homo sapiens
aggagcaattttactaccccccatgtaatttttacttcttt

SEQ ID NO: 241 - DNA, 41 bp, Homo sapiens
gagcaattttactaccccacgtgtaatttttacttctttat

SEQ ID NO: 242 - DNA, 41 bp, Homo sapiens
tatcaaacagtcttttgtgaaaacagagtcacaactgggct

SEQ ID NO: 243 - DNA, 41 bp, Homo sapiens
accagctgagtgctctcagccggagctcagctgtccacctt

SEQ ID NO: 244 - DNA, 41 bp, Homo sapiens
agcagcaaggccaggcagcacgctcctccgcatgccttacg

SEQ ID NO: 245 - DNA, 41 bp, Homo sapiens
ctgcctgttgcctgtaacagcaagacagaaagcagagtggc

SEQ ID NO: 246 - DNA, 41 bp, Homo sapiens
cacgaggctcctgggtcccctcaaatggatccttacctgcg

SEQ ID NO: 247 - DNA, 41 bp, Homo sapiens
aaagctcactttttcctcaactgcttcatcccaactgacca

SEQ ID NO: 248 - DNA, 41 bp, Homo sapiens
gcatccactctattatggaatttttcttcattcttgttact

SEQ ID NO: 249 - DNA, 41 bp, Homo sapiens
ggctgaggctcagagagattgtaaccttgtgccaggctcaa

SEQ ID NO: 250 - DNA, 41 bp, Homo sapiens
cggtggtcctagaggcaaagagcagtagggtgggaaatcaa

SEQ ID NO: 251 - DNA, 41 bp, Homo sapiens
gagcctgcagatggaggagaagtgggtggcagaaaaaggag

SEQ ID NO: 252 - DNA, 41 bp, Homo sapiens
aatatcaggcagtgatgggtaggccaggaccagaaagggca

SEQ ID NO: 253 - DNA, 41 bp, Homo sapiens
cagccctggtcccagaaagcggtaggaacccagtgaaagaa

SEQ ID NO: 254 - DNA, 41 bp, Homo sapiens
tcgacttggattcggaggacaaggctggagacaatagcaag

SEQ ID NO: 255 - DNA, 41 bp, Homo sapiens
cttggattcggaggacgaggttggagacaatagcaagaggt

SEQ ID NO: 256 - DNA, 41 bp, Homo sapiens
acgcccattgcagcagggcacggagcagggccttgaaagtc

SEQ ID NO: 257 - DNA, 41 bp, Homo sapiens
gcccattgcagcagggcaggaagcagggccttgaaagtcca

SEQ ID NO: 258 - DNA, 41 bp, Homo sapiens
gcctggagaaagtagcgcccatcagtccacatggctgcatg

SEQ ID NO: 259 - DNA, 41 bp, Homo sapiens
gactgttctgcctgcttagctttgtgctaggctcagcgggg

SEQ ID NO: 260 - DNA, 41 bp, Homo sapiens
atttcaaaaccagcctgccctcagcaattcaacgtccagtt

SEQ ID NO: 261 - DNA, 41 bp, Homo sapiens
taggtgtttatctttgtttacagtagaaaaaagcaggagaa

SEQ ID NO: 262 - DNA, 41 bp, Homo sapiens
aaagcaggagaagtgtatgggcagttaaataaactataata

SEQ ID NO: 263 - DNA, 41 bp, Homo sapiens
agatgacaggctgggcgcagcggctcacgcctttaattcca

SEQ ID NO: 264 - DNA, 41 bp, Homo sapiens
agtttctagaaggacagtcatccaagtgttaagagtggtta

SEQ ID NO: 265 - DNA, 41 bp, Homo sapiens
agctctcagaagccagttcacaggctgagtcccctccctca

SEQ ID NO: 266 - DNA, 41 bp, Homo sapiens
gctctcagaagccagttcaggggctgagtcccctccctcag

SEQ ID NO: 267 - DNA, 41 bp, Homo sapiens
aagtctgtcaccttctggacatgggcttttttgcactcatc

SEQ ID NO: 268 - DNA, 41 bp, Homo sapiens
ctgcttccctggccctttctaccttggacgcaggagtcccc

SEQ ID NO: 269 - DNA, 41 bp, Homo sapiens
ctttagcattacaaaatccaatgtccctcccaaacaatgct

SEQ ID NO: 270 - DNA, 41 bp, Homo sapiens
aatgagcctcccacttcttgttcccagttatgggtgctcaa

SEQ ID NO: 271 - DNA, 41 bp, Homo sapiens
ggtctctgaagataattagggactagattcctgcacctcaa

SEQ ID NO: 272 - DNA, 41 bp, Homo sapiens
gcattcctctctgggaaatcgccttcctcgaacccaaagag

SEQ ID NO: 273 - DNA, 41 bp, Homo sapiens
tgggaatcagcttgctggagagaaggggaccgaattaagga

SEQ ID NO: 274 - DNA, 41 bp, Homo sapiens
accctctgtctgctcgagcctaggaaaggcctgaaggaaga

SEQ ID NO: 275 - DNA, 41 bp, Homo sapiens
gaggccggggaaagagccctgcctctctcccttgtccctcc

SEQ ID NO: 276 - DNA, 41 bp, Homo sapiens
gcttcctggaagaggcacttagtgtgcttgggcttgtagct

SEQ ID NO: 277 - DNA, 41 bp, Homo sapiens
ctgcaagtttctctgttctgtcccaggaactgcagtggtga

SEQ ID NO: 278 - DNA, 41 bp, Homo sapiens
tataaagaaatggaccttgggagaaggaggggcggtgggac

SEQ ID NO: 279 - DNA, 41 bp, Homo sapiens
agggaaccaggactaactttacctgaatcacaattttttcc

SEQ ID NO: 280 - DNA, 41 bp, Homo sapiens
gatacccaaggtgggtttgctaggccccagcccaagccagg

SEQ ID NO: 281 - DNA, 41 bp, Homo sapiens
cagaggcgccagctactagaagagttcgagtggcttcaaca

SEQ ID NO: 282 - DNA, 41 bp, Homo sapiens
tccaagacctatggagaagggcccaggccccaggaacacag

SEQ ID NO: 283 - DNA, 41 bp, Homo sapiens
attggccgttagccacctggttccacatcctgctaagacgt

SEQ ID NO: 284 - DNA, 41 bp, Homo sapiens
aaacccattgctctgatggctttgaagatgatggaactttc

SEQ ID NO: 285 - DNA, 41 bp, Homo sapiens
ggcctcctccttcctgtctttgcctttgttttgggctgcta

SEQ ID NO: 286 - DNA, 41 bp, Homo sapiens
gagacatcttcaatttcttccttgttggagtggtctgcata

SEQ ID NO: 287 - DNA, 41 bp, Homo sapiens
acatcttcaatttcttctttattggagtggtctgcatagga

SEQ ID NO: 288 - DNA, 41 bp, Homo sapiens
aagaagaaaacaaaattagcttaaggagaaatttttgaaag

SEQ ID NO: 289 - DNA, 41 bp, Homo sapiens
tttttgaaagcatctgcgaacgcaactcatatacacacgca

SEQ ID NO: 290 - DNA, 41 bp, Homo sapiens
tccctggcctacttgattcacgtgctctgtattttcttctt

SEQ ID NO: 291 - DNA, 41 bp, Homo sapiens
tttcagaactaaccaataaacagattccatgttttcttgtt

SEQ ID NO: 292 - DNA, 41 bp, Homo sapiens
caaagagccctgaggatgagagacgtgggatcattccccgg

SEQ ID NO: 293 - DNA, 41 bp, Homo sapiens
atcattccccggtttccccccggccaggtgctaggtcgttt

SEQ ID NO: 294 - DNA, 41 bp, Homo sapiens
aggcggctataggtggcaagtggagctgtccagcactagtg

SEQ ID NO: 295 - DNA, 39 bp, Homo sapiens
tgtaaaacgacggccagtagccaactaaaggggcataaa

SEQ ID NO: 296 - DNA, 39 bp, Homo sapiens
tgtaaaacgacggccagtctggactttctgggtttggtt

SEQ ID NO: 297 - DNA, 39 bp, Homo sapiens
tgtaaaacgacggccagtctttgaaggtgacactgagcc

SEQ ID NO: 298 - DNA, 39 bp, Homo sapiens
tgtaaaacgacggccagtttcttctgcctctggcaaata

SEQ ID NO: 299 - DNA, 39 bp, Homo sapiens
tgtaaaacgacggccagtgtgccacctgtactcacagtg

SEQ ID NO: 300 - DNA, 39 bp, Homo sapiens
tgtaaaacgacggccagtcggatctttaagcaataggcc

SEQ ID NO: 301 - DNA, 39 bp, Homo sapiens; identical to SEQ ID NO: 300

SEQ ID NO: 302 - DNA, 39 bp, Homo sapiens
tgtaaaacgacggccagttttgtatctcccacttcccaa

SEQ ID NO: 303 - DNA, 39 bp, Homo sapiens
tgtaaaacgacggccagttgaggacaactggacgatttt

SEQ ID NO: 304 - DNA, 39 bp, Homo sapiens; identical to SEQ ID NO: 303

SEQ ID NO: 305 - DNA, 39 bp, Homo sapiens
tgtaaaacgacggccagtagagctgagagatgccagttg

SEQ ID NO: 306 - DNA, 39 bp, Homo sapiens
tgtaaaacgacggccagtgagatgttccctttgtctccc

SEQ ID NO: 307 - DNA, 39 bp, Homo sapiens
tgtaaaacgacggccagtagcatgtgtttattcatccgg

SEQ ID NO: 308 - DNA, 36 bp, Homo sapiens
tgtaaaacgacggccagtgtcccagactccacggtg

SEQ ID NO: 309 - DNA, 36 bp, Homo sapiens; identical to SEQ ID NO: 308

SEQ ID NO: 310 - DNA, 36 bp, Homo sapiens; identical to SEQ ID NO: 308

SEQ ID NO: 311 - DNA, 39 bp, Homo sapiens
tgtaaaacgacggccagtcctgggtcatcaggaactaga

SEQ ID NO: 312 - DNA, 39 bp, Homo sapiens
tgtaaaacgacggccagtaaagtctggctactcggcttc

SEQ ID NO: 313 - DNA, 39 bp, Homo sapiens; identical to SEQ ID NO: 312

SEQ ID NO: 314 - DNA, 39 bp, Homo sapiens; identical to SEQ ID NO: 312

SEQ ID NO: 315 - DNA, 39 bp, Homo sapiens; identical to SEQ ID NO: 312

SEQ ID NO: 316 - DNA, 39 bp, Homo sapiens; identical to SEQ ID NO: 312

SEQ ID NO: 317 - DNA, 39 bp, Homo sapiens
tgtaaaacgacggccagttggctgatttttgtccacttc

SEQ ID NO: 318 - DNA, 39 bp, Homo sapiens; identical to SEQ ID NO: 317

SEQ ID NO: 319 - DNA, 39 bp, Homo sapiens; identical to SEQ ID NO: 317

SEQ ID NO: 320 - DNA, 39 bp, Homo sapiens; identical to SEQ ID NO: 317

SEQ ID NO: 321 - DNA, 39 bp, Homo sapiens
tgtaaaacgacggccagtaatactgcctccacctcaaca

SEQ ID NO: 322 - DNA, 39 bp, Homo sapiens; identical to SEQ ID NO: 321

SEQ ID NO: 323 - DNA, 39 bp, Homo sapiens; identical to SEQ ID NO: 321

SEQ ID NO: 324 - DNA, 39 bp, Homo sapiens; identical to SEQ ID NO: 321

SEQ ID NO: 325 - DNA, 39 bp, Homo sapiens
tgtaaaacgacggccagtcagagaacactgccaggatgt

SEQ ID NO: 326 - DNA, 39 bp, Homo sapiens
tgtaaaacgacggccagtcatctgtgaaatgggtgtgtg

SEQ ID NO: 327 - DNA, 39 bp, Homo sapiens; identical to SEQ ID NO: 326

SEQ ID NO: 328 - DNA, 39 bp, Homo sapiens; identical to SEQ ID NO: 326

SEQ ID NO: 329 - DNA, 39 bp, Homo sapiens
tgtaaaacgacggccagtcagcaaagtggagattggaac

SEQ ID NO: 330 - DNA, 39 bp, Homo sapiens; identical to SEQ ID NO: 329

SEQ ID NO: 331 - DNA, 39 bp, Homo sapiens; identical to SEQ ID NO: 329

SEQ ID NO: 332 - DNA, 38 bp, Homo sapiens
tgtaaaacgacggccagtcagaccctgccttcagtgag

SEQ ID NO: 333 - DNA, 38 bp, Homo sapiens; identical to SEQ ID NO: 332

SEQ ID NO: 334 - DNA, 39 bp, Homo sapiens
tgtaaaacgacggccagtacacatcattcagcaccatga

SEQ ID NO: 335 - DNA, 39 bp, Homo sapiens
tgtaaaacgacggccagtgatttcaacaccatcgtgacc

SEQ ID NO: 336 - DNA, 38 bp, Homo sapiens
tgtaaaacgacggccagtgtcaccagtccccagttctc

SEQ ID NO: 337 - DNA, 38 bp, Homo sapiens; identical to SEQ ID NO: 336

SEQ ID NO: 338 - DNA, 38 bp, Homo sapiens; identical to SEQ ID NO: 336

SEQ ID NO: 339 - DNA, 38 bp, Homo sapiens; identical to SEQ ID NO: 336

SEQ ID NO: 340 - DNA, 38 bp, Homo sapiens; identical to SEQ ID NO: 336

SEQ ID NO: 341 - DNA, 39 bp, Homo sapiens
tgtaaaacgacggccagtgtgtgtgtgaggacgtaccct

SEQ ID NO: 342 - DNA, 39 bp, Homo sapiens; identical to SEQ ID NO: 341

SEQ ID NO: 343 - DNA, 39 bp, Homo sapiens
tgtaaaacgacggccagtcctgcaagctaagtccttcct

SEQ ID NO: 344 - DNA, 39 bp, Homo sapiens
tgtaaaacgacggccagtctccaccagctcagtcttgtc

SEQ ID NO: 345 - DNA, 36 bp, Homo sapiens
tgtaaaacgacggccagttcaggccaggcagagaac

SEQ ID NO: 346 - DNA, 39 bp, Homo sapiens
tgtaaaacgacggccagtgggaacagcatcagaactgag

SEQ ID NO: 347 - DNA, 39 bp, Homo sapiens; identical to SEQ ID NO: 346

SEQ ID NO: 348 - DNA, 39 bp, Homo sapiens; identical to SEQ ID NO: 346

SEQ ID NO: 349 - DNA, 39 bp, Homo sapiens; identical to SEQ ID NO: 346

SEQ ID NO: 350 - DNA, 39 bp, Homo sapiens
tgtaaaacgacggccagttgcctctcaggtttgttgaac

SEQ ID NO: 351 - DNA, 39 bp, Homo sapiens; identical to SEQ ID NO: 350

SEQ ID NO: 352 - DNA, 39 bp, Homo sapiens; identical to SEQ ID NO: 350

SEQ ID NO: 353 - DNA, 39 bp, Homo sapiens; identical to SEQ ID NO: 350

SEQ ID NO: 354 - DNA, 39 bp, Homo sapiens; identical to SEQ ID NO: 350

SEQ ID NO: 355 - DNA, 39 bp, Homo sapiens
tgtaaaacgacggccagtttagcacaaaatggggtgaag

SEQ ID NO: 356 - DNA, 39 bp, Homo sapiens; identical to SEQ ID NO: 355

SEQ ID NO: 357 - DNA, 39 bp, Homo sapiens; identical to SEQ ID NO: 355

SEQ ID NO: 358 - DNA, 39 bp, Homo sapiens; identical to SEQ ID NO: 355

SEQ ID NO: 359 - DNA, 40 bp, Homo sapiens
tgtaaaacgacggccagtgattacacatgtgagccaccat

SEQ ID NO: 360 - DNA, 39 bp, Homo sapiens
tgtaaaacgacggccagttagtcacctgaggccaaaaga

SEQ ID NO: 361 - DNA, 39 bp, Homo sapiens
tgtaaaacgacggccagtacacacaatttgtttgtgcga

SEQ ID NO: 362 - DNA, 39 bp, Homo sapiens
tgtaaaacgacggccagtggccaaataagggatctgaag

SEQ ID NO: 363 - DNA, 39 bp, Homo sapiens
tgtaaaacgacggccagtaaaggtgggtaatgaagtggg

SEQ ID NO: 364 - DNA, 39 bp, Homo sapiens; identical to SEQ ID NO: 363

SEQ ID NO: 365 - DNA, 39 bp, Homo sapiens; identical to SEQ ID NO: 363

SEQ ID NO: 366 - DNA, 41 bp, Homo sapiens
tgtaaaacgacggccagtccaacctggcatttctaatactg

SEQ ID NO: 367 - DNA, 41 bp, Homo sapiens; identical to SEQ ID NO: 366

SEQ ID NO: 368 - DNA, 39 bp, Homo sapiens
tgtaaaacgacggccagttggatgttacttatgcctcgc

SEQ ID NO: 369 - DNA, 39 bp, Homo sapiens; identical to SEQ ID NO: 368

SEQ ID NO: 370 - DNA, 39 bp, Homo sapiens
tgtaaaacgacggccagtggttttggatgaccctttgtc

SEQ ID NO: 371 - DNA, 39 bp, Homo sapiens; identical to SEQ ID NO: 370

SEQ ID NO: 372 - DNA, 39 bp, Homo sapiens
tgtaaaacgacggccagtcctgttctttcctaccaccaa

SEQ ID NO: 373 - DNA, 39 bp, Homo sapiens; identical to SEQ ID NO: 372

SEQ ID NO: 374 - DNA, 39 bp, Homo sapiens; identical to SEQ ID NO: 372

SEQ ID NO: 375 - DNA, 39 bp, Homo sapiens; identical to SEQ ID NO: 372

SEQ ID NO: 376 - DNA, 39 bp, Homo sapiens
tgtaaaacgacggccagtgcaacattgcaagatctcacc

SEQ ID NO: 377 - DNA, 42 bp, Homo sapiens
tgtaaaacgacggccagtggtgatccacatctttaactgtga

SEQ ID NO: 378 - DNA, 42 bp, Homo sapiens; identical to SEQ ID NO: 377

SEQ ID NO: 379 - DNA, 42 bp, Homo sapiens; identical to SEQ ID NO: 377

SEQ ID NO: 380 - DNA, 39 bp, Homo sapiens
tgtaaaacgacggccagttgctgatggggacttcattta

SEQ ID NO: 381 - DNA, 39 bp, Homo sapiens; identical to SEQ ID NO: 380

SEQ ID NO: 382 - DNA, 39 bp, Homo sapiens
tgtaaaacgacggccagtaaggaaaggggaaagatgtca

SEQ ID NO: 383 - DNA, 39 bp, Homo sapiens; identical to SEQ ID NO: 382

SEQ ID NO: 384 - DNA, 39 bp, Homo sapiens
tgtaaaacgacggccagtggaagggaaaagtattgccaa

SEQ ID NO: 385 - DNA, 39 bp, Homo sapiens; identical to SEQ ID NO: 384

SEQ ID NO: 386 - DNA, 39 bp, Homo sapiens
tgtaaaacgacggccagttatggagactcaccccatgag

SEQ ID NO: 387 - DNA, 39 bp, Homo sapiens
tgtaaaacgacggccagtataagcctgactccacagcaa

SEQ ID NO: 388 - DNA, 39 bp, Homo sapiens; identical to SEQ ID NO: 387

SEQ ID NO: 389 - DNA, 39 bp, Homo sapiens
tgtaaaacgacggccagtaaacttccagccaaggactgt

SEQ ID NO: 390 - DNA, 39 bp, Homo sapiens; identical to SEQ ID NO: 389

SEQ ID NO: 391 - DNA, 39 bp, Homo sapiens
tgtaaaacgacggccagttaagaagaggctcccaaaagg

SEQ ID NO: 392 - DNA, 39 bp, Homo sapiens
tgtaaaacgacggccagtaaattgttgggaagctcaggt

SEQ ID NO: 393 - DNA, 39 bp, Homo sapiens; identical to SEQ ID NO: 392

SEQ ID NO: 394 - DNA, 39 bp, Homo sapiens; identical to SEQ ID NO: 392

SEQ ID NO: 395 - DNA, 39 bp, Homo sapiens
tgtaaaacgacggccagtaacccaaaaagcatccactct

SEQ ID NO: 396 - DNA, 39 bp, Homo sapiens
tgtaaaacgacggccagtcaataactcctcctggaaggc

SEQ ID NO: 397 - DNA, 39 bp, Homo sapiens; identical to SEQ ID NO: 396

SEQ ID NO: 398 - DNA, 39 bp, Homo sapiens
tgtaaaacgacggccagtctcatccatggagaccacagt

SEQ ID NO: 399 - DNA, 39 bp, Homo sapiens
tgtaaaacgacggccagtgggaaaatgttaaaagggcaa

SEQ ID NO: 400 - DNA, 39 bp, Homo sapiens
tgtaaaacgacggccagtcttcttattccctggccactc

SEQ ID NO: 401 - DNA, 39 bp, Homo sapiens
tgtaaaacgacggccagtgtagggcaaaatcctcctgtc

SEQ ID NO: 402 - DNA, 39 bp, Homo sapiens; identical to SEQ ID NO: 401

SEQ ID NO: 403 - DNA, 39 bp, Homo sapiens; identical to SEQ ID NO: 401

SEQ ID NO: 404 - DNA, 39 bp, Homo sapiens; identical to SEQ ID NO: 401

SEQ ID NO: 405 - DNA, 39 bp, Homo sapiens
tgtaaaacgacggccagttccttggattctcaaaagggt

SEQ ID NO: 406 - DNA, 39 bp, Homo sapiens
tgtaaaacgacggccagtgtcaatgtggagcatctggtt

SEQ ID NO: 407 - DNA, 39 bp, Homo sapiens
tgtaaaacgacggccagtatcggttcggtcacatactca

SEQ ID NO: 408 - DNA, 39 bp, Homo sapiens; identical to SEQ ID NO: 407

SEQ ID NO: 409 - DNA, 41 bp, Homo sapiens
tgtaaaacgacggccagttgactggttacctctttcactcc

SEQ ID NO: 410 - DNA, 41 bp, Homo sapiens; identical to SEQ ID NO: 409

SEQ ID NO: 411 - DNA, 36 bp, Homo sapiens
tgtaaaacgacggccagtatcagccaggtgtggtgg

SEQ ID NO: 412 - DNA, 39 bp, Homo sapiens
tgtaaaacgacggccagtgtgaagcagatgcctggttag

SEQ ID NO: 413 - DNA, 39 bp, Homo sapiens; identical to SEQ ID NO: 412

SEQ ID NO: 414 - DNA, 39 bp, Homo sapiens; identical to SEQ ID NO: 412

SEQ ID NO: 415 - DNA, 39 bp, Homo sapiens; identical to SEQ ID NO: 412

SEQ ID NO: 416 - DNA, 39 bp, Homo sapiens
tgtaaaacgacggccagtgggaagcagaaacaggaagac

SEQ ID NO: 417 - DNA, 39 bp, Homo sapiens
tgtaaaacgacggccagtgggtctcccagggtaagttct

SEQ ID NO: 418 - DNA, 39 bp, Homo sapiens; identical to SEQ ID NO: 417

SEQ ID NO: 419 - DNA, 38 bp, Homo sapiens
tgtaaaacgacggccagtctccaggctaggtgagcatc

SEQ ID NO: 420 - DNA, 38 bp, Homo sapiens
tgtaaaacgacggccagtaggaggatgaggagggaaaa

SEQ ID NO: 421 - DNA, 39 bp, Homo sapiens
tgtaaaacgacggccagtaaaagcaggctgattgagacc

SEQ ID NO: 422 - DNA, 39 bp, Homo sapiens; identical to SEQ ID NO: 421

SEQ ID NO: 423 - DNA, 39 bp, Homo sapiens
tgtaaaacgacggccagtctgcaaaagatacggttgctc

SEQ ID NO: 424 - DNA, 39 bp, Homo sapiens
tgtaaaacgacggccagttgattcaggacacctttctgc

SEQ ID NO: 425 - DNA, 39 bp, Homo sapiens
tgtaaaacgacggccagtaggaaatttgaggccatcact

SEQ ID NO: 426 - DNA, 39 bp, Homo sapiens
tgtaaaacgacggccagtgcaaagggaaccaggactaac

SEQ ID NO: 427 - DNA, 39 bp, Homo sapiens
tgtaaaacgacggccagtcttcctttgacctccaggaac

SEQ ID NO: 428 - DNA, 39 bp, Homo sapiens
tgtaaaacgacggccagtggactatggtgacagctggag

SEQ ID NO: 429 - DNA, 39 bp, Homo sapiens
tgtaaaacgacggccagtagccacagctacaatgctgtt

SEQ ID NO: 430 - DNA, 39 bp, Homo sapiens
tgtaaaacgacggccagtagagcccaaacctatcaccac

SEQ ID NO: 431 - DNA, 39 bp, Homo sapiens
tgtaaaacgacggccagtgatgttttgccgacatgtttt

SEQ ID NO: 432 - DNA, 39 bp, Homo sapiens
tgtaaaacgacggccagtttcctgtctttgaggagctca

SEQ ID NO: 433 - DNA, 39 bp, Homo sapiens
tgtaaaacgacggccagtaacacccatcacttcacaagg

SEQ ID NO: 434 - DNA, 39 bp, Homo sapiens; identical to SEQ ID NO: 433

SEQ ID NO: 435 - DNA, 39 bp, Homo sapiens; identical to SEQ ID NO: 433

SEQ ID NO: 436 - DNA, 39 bp, Homo sapiens; identical to SEQ ID NO: 433

SEQ ID NO: 437 - DNA, 39 bp, Homo sapiens
tgtaaaacgacggccagtaggcttcctgagcctttaaaa

SEQ ID NO: 438 - DNA, 37 bp, Homo sapiens
tgtaaaacgacggccagtccagaacatgggtcaccaa

SEQ ID NO: 439 - DNA, 39 bp, Homo sapiens
tgtaaaacgacggccagtggaaggactggtttgcaaaga

SEQ ID NO: 440 - DNA, 39 bp, Homo sapiens; identical to SEQ ID NO: 439

SEQ ID NO: 441 - DNA, 39 bp, Homo sapiens; identical to SEQ ID NO: 439

SEQ ID NO: 442 - DNA, 39 bp, Homo sapiens
caggaaacagctatgacctcctcctctccctccctttat

SEQ ID NO: 443 - DNA, 39 bp, Homo sapiens
caggaaacagctatgaccttcagaagctggtaggagcaa

SEQ ID NO: 444 - DNA, 38 bp, Homo sapiens
caggaaacagctatgacccatgacgctgggctaatttt

SEQ ID NO: 445 - DNA, 39 bp, Homo sapiens
caggaaacagctatgacccactccctcccacatccttat

SEQ ID NO: 446 - DNA, 39 bp, Homo sapiens
caggaaacagctatgacccctcattcaatgtgttgctca

SEQ ID NO: 447 - DNA, 39 bp, Homo sapiens
caggaaacagctatgaccaagaagtgccaaatgaaggct

SEQ ID NO: 448 - DNA, 39 bp, Homo sapiens; identical to SEQ ID NO: 447

SEQ ID NO: 449 - DNA, 39 bp, Homo sapiens
caggaaacagctatgacccatctacctgctttgatccca

SEQ ID NO: 450 - DNA, 39 bp, Homo sapiens
caggaaacagctatgaccctccatgactcacaagggaga

SEQ ID NO: 451 - DNA, 39 bp, Homo sapiens; identical to SEQ ID NO: 450

SEQ ID NO: 452 - DNA, 39 bp, Homo sapiens
caggaaacagctatgacctctccaaactctgtctctggc

SEQ ID NO: 453 - DNA, 39 bp, Homo sapiens
caggaaacagctatgacctcagtcctttcccaagaggat

SEQ ID NO: 454 - DNA, 39 bp, Homo sapiens
caggaaacagctatgaccaccactgtcccctttacagct

SEQ ID NO: 455 - DNA, 39 bp, Homo sapiens
caggaaacagctatgaccctcccctcaaggacaaagtct

SEQ ID NO: 456 - DNA, 39 bp, Homo sapiens; identical to SEQ ID NO: 455

SEQ ID NO: 457 - DNA, 39 bp, Homo sapiens; identical to SEQ ID NO: 455

SEQ ID NO: 458 - DNA, 39 bp, Homo sapiens
caggaaacagctatgaccgggactctaaagtgggagtgg

SEQ ID NO: 459 - DNA, 39 bp, Homo sapiens
caggaaacagctatgaccgtgactcccccacatcctagt

SEQ ID NO: 460 - DNA, 39 bp, Homo sapiens; identical to SEQ ID NO: 459

SEQ ID NO: 461 - DNA, 39 bp, Homo sapiens; identical to SEQ ID NO: 459

SEQ ID NO: 462 - DNA, 39 bp, Homo sapiens; identical to SEQ ID NO: 459

SEQ ID NO: 463 - DNA, 39 bp, Homo sapiens; identical to SEQ ID NO: 459

SEQ ID NO: 464 - DNA, 39 bp, Homo sapiens
caggaaacagctatgaccgttgagaggatgcaggcatag

SEQ ID NO: 465 - DNA, 39 bp, Homo sapiens; identical to SEQ ID NO: 464

SEQ ID NO: 466 - DNA, 39 bp, Homo sapiens; identical to SEQ ID NO: 464

SEQ ID NO: 467 - DNA, 39 bp, Homo sapiens; identical to SEQ ID NO: 464

SEQ ID NO: 468 - DNA, 38 bp, Homo sapiens
caggaaacagctatgacccctcctctgccagtcttcat

SEQ ID NO: 469 - DNA, 38 bp, Homo sapiens; identical to SEQ ID NO: 468

SEQ ID NO: 470 - DNA, 38 bp, Homo sapiens; identical to SEQ ID NO: 468

SEQ ID NO: 471 - DNA, 38 bp, Homo sapiens; identical to SEQ ID NO: 468

SEQ ID NO: 472 - DNA, 39 bp, Homo sapiens
caggaaacagctatgaccctctagacttctatgggcccc

SEQ ID NO: 473 - DNA, 39 bp, Homo sapiens
caggaaacagctatgaccctcagctgcagagagaccact

SEQ ID NO: 474 - DNA, 39 bp, Homo sapiens; identical to SEQ ID NO: 473

SEQ ID NO: 475 - DNA, 39 bp, Homo sapiens; identical to SEQ ID NO: 473

SEQ ID NO: 476 - DNA, 39 bp, Homo sapiens
caggaaacagctatgaccgtagacagaccctccctccag

SEQ ID NO: 477 - DNA, 39 bp, Homo sapiens; identical to SEQ ID NO: 476

SEQ ID NO: 478 - DNA, 39 bp, Homo sapiens; identical to SEQ ID NO: 476

SEQ ID NO: 479 - DNA, 39 bp, Homo sapiens
caggaaacagctatgaccgtacctgggtgctgactatgc

SEQ ID NO: 480 - DNA, 39 bp, Homo sapiens; identical to SEQ ID NO: 479

SEQ ID NO: 481 - DNA, 39 bp, Homo sapiens
caggaaacagctatgaccggtcacgatggtgttgaaatc

SEQ ID NO: 482 - DNA, 39 bp, Homo sapiens
caggaaacagctatgaccactcccaaaatcaggtgagtg

SEQ ID NO: 483 - DNA, 39 bp, Homo sapiens
caggaaacagctatgaccaggtttagcttgattggcctc

SEQ ID NO: 484 - DNA, 39 bp, Homo sapiens; identical to SEQ ID NO: 483

SEQ ID NO: 485 - DNA, 39 bp, Homo sapiens; identical to SEQ ID NO: 483

SEQ ID NO: 486 - DNA, 39 bp, Homo sapiens; identical to SEQ ID NO: 483

SEQ ID NO: 487 - DNA, 39 bp, Homo sapiens; identical to SEQ ID NO: 483

SEQ ID NO: 488 - DNA, 38 bp, Homo sapiens
caggaaacagctatgaccctggtagcatcctcagagcc

SEQ ID NO: 489 - DNA, 38 bp, Homo sapiens; identical to SEQ ID NO: 488

SEQ ID NO: 490 - DNA, 39 bp, Homo sapiens
caggaaacagctatgacctcatcatcatccacagcaaga

SEQ ID NO: 491 - DNA, 39 bp, Homo sapiens
caggaaacagctatgaccaatcatcgcactgtcagtggt

SEQ ID NO: 492 - DNA, 39 bp, Homo sapiens
caggaaacagctatgaccgtgttcttaataggagccggg

SEQ ID NO: 493 - DNA, 38 bp, Homo sapiens
caggaaacagctatgacccagccgggaatgatttttaa

SEQ ID NO: 494 - DNA, 38 bp, Homo sapiens; identical to SEQ ID NO: 493

SEQ ID NO: 495 - DNA, 38 bp, Homo sapiens; identical to SEQ ID NO: 493

SEQ ID NO: 496 - DNA, 38 bp, Homo sapiens; identical to SEQ ID NO: 493

SEQ ID NO: 497 - DNA, 46 bp, Homo sapiens
caggaaacagctatgaccaaggtgtgtattttattttgtatggtaa

SEQ ID NO: 498 - DNA, 46 bp, Homo sapiens; identical to SEQ ID NO: 497

SEQ ID NO: 499 - DNA, 46 bp, Homo sapiens; identical to SEQ ID NO: 497

SEQ ID NO: 500 - DNA, 46 bp, Homo sapiens; identical to SEQ ID NO: 497

SEQ ID NO: 501 - DNA, 46 bp, Homo sapiens; identical to SEQ ID NO: 497

SEQ ID NO: 502 - DNA, 39 bp, Homo sapiens
caggaaacagctatgacccaacctgttctctcagttccc

SEQ ID NO: 503 - DNA, 39 bp, Homo sapiens; identical to SEQ ID NO: 502

SEQ ID NO: 504 - DNA, 39 bp, Homo sapiens; identical to SEQ ID NO: 502

SEQ ID NO: 505 - DNA, 39 bp, Homo sapiens; identical to SEQ ID NO: 502

SEQ ID NO: 506 - DNA, 39 bp, Homo sapiens
caggaaacagctatgacctgcctggctgagacttcttaa

SEQ ID NO: 507 - DNA, 39 bp, Homo sapiens
caggaaacagctatgaccgaatttggcttgcagtggtta

SEQ ID NO: 508 - DNA, 39 bp, Homo sapiens
caggaaacagctatgacccaaaggagcaaaccaaacaaa

SEQ ID NO: 509 - DNA, 39 bp, Homo sapiens
caggaaacagctatgaccattgctgcaatgtaatgtccc

SEQ ID NO: 510 - DNA, 39 bp, Homo sapiens
caggaaacagctatgacccagttgccacagatgacaaga

SEQ ID NO: 511 - DNA, 39 bp, Homo sapiens; identical to SEQ ID NO: 510

SEQ ID NO: 512 - DNA, 39 bp, Homo sapiens; identical to SEQ ID NO: 510

SEQ ID NO: 513 - DNA, 39 bp, Homo sapiens
caggaaacagctatgaccatcaggtgcaaggcctacttc

SEQ ID NO: 514 - DNA, 39 bp, Homo sapiens; identical to SEQ ID NO: 513

SEQ ID NO: 515 - DNA, 39 bp, Homo sapiens
caggaaacagctatgaccagtagcccctgccctattgta

SEQ ID NO: 516 - DNA, 39 bp, Homo sapiens; identical to SEQ ID NO: 515

SEQ ID NO: 517 - DNA, 39 bp, Homo sapiens
caggaaacagctatgaccatccctgcattagtcacatgg

SEQ ID NO: 518 - DNA, 39 bp, Homo sapiens; identical to SEQ ID NO: 517

SEQ ID NO: 519 - DNA, 39 bp, Homo sapiens
caggaaacagctatgaccatattcctcccaaaccccatt

SEQ ID NO: 520 - DNA, 39 bp, Homo sapiens; identical to SEQ ID NO: 519

SEQ ID NO: 521 - DNA, 39 bp, Homo sapiens; identical to SEQ ID NO: 519

SEQ ID NO: 522 - DNA, 39 bp, Homo sapiens; identical to SEQ ID NO: 519

SEQ ID NO: 523 - DNA, 39 bp, Homo sapiens
caggaaacagctatgacctgaatggctgtgagatgagaa

SEQ ID NO: 524 - DNA, 41 bp, Homo sapiens
caggaaacagctatgaccaaaaatgtcctagttcctctcca

SEQ ID NO: 525 - DNA, 41 bp, Homo sapiens; identical to SEQ ID NO: 524

SEQ ID NO: 526 - DNA, 41 bp, Homo sapiens; identical to SEQ ID NO: 524

SEQ ID NO: 527 - DNA, 41 bp, Homo sapiens
caggaaacagctatgaccgaaaagccatctctaactgttgc

SEQ ID NO: 528 - DNA, 41 bp, Homo sapiens; identical to SEQ ID NO: 527

SEQ ID NO: 529 - DNA, 39 bp, Homo sapiens
caggaaacagctatgaccgtatttccggggaaacagaac

SEQ ID NO: 530 - DNA, 39 bp, Homo sapiens; identical to SEQ ID NO: 529

SEQ ID NO: 531 - DNA, 39 bp, Homo sapiens
caggaaacagctatgacccgttctggagccacacataat

SEQ ID NO: 532 - DNA, 39 bp, Homo sapiens; identical to SEQ ID NO: 531

SEQ ID NO: 533 - DNA, 39 bp, Homo sapiens
caggaaacagctatgaccttagcaggcaggattttctga

SEQ ID NO: 534 - DNA, 39 bp, Homo sapiens
caggaaacagctatgacctctcacttgtccacatgcttg

SEQ ID NO: 535 - DNA, 39 bp, Homo sapiens; identical to SEQ ID NO: 534

SEQ ID NO: 536 - DNA, 39 bp, Homo sapiens
caggaaacagctatgaccagctcttgtgtgtcctcctcc

SEQ ID NO: 537 - DNA, 39 bp, Homo sapiens; identical to SEQ ID NO: 536

SEQ ID NO: 538 - DNA, 39 bp, Homo sapiens
caggaaacagctatgaccttgctagttgaatgaggctgg

SEQ ID NO: 539 - DNA, 39 bp, Homo sapiens
caggaaacagctatgaccgtgagagcctttgggagttct

SEQ ID NO: 540 - DNA, 39 bp, Homo sapiens; identical to SEQ ID NO: 539

SEQ ID NO: 541 - DNA, 39 bp, Homo sapiens; identical to SEQ ID NO: 539

SEQ ID NO: 542 - DNA, 39 bp, Homo sapiens
caggaaacagctatgacctgcaggtacagccttctcagt

SEQ ID NO: 543 - DNA, 39 bp, Homo sapiens
caggaaacagctatgaccaaaaggttgttgccttggtct

SEQ ID NO: 544 - DNA, 39 bp, Homo sapiens; identical to SEQ ID NO: 543

SEQ ID NO: 545 - DNA, 39 bp, Homo sapiens
caggaaacagctatgaccagagaaaaggggcttggtttt

SEQ ID NO: 546 - DNA, 39 bp, Homo sapiens
caggaaacagctatgaccactccaagctgaagctcttcc

SEQ ID NO: 547 - DNA, 39 bp, Homo sapiens
caggaaacagctatgacccctgcctgtttaagtagcgtg

SEQ ID NO: 548 - DNA, 39 bp, Homo sapiens
caggaaacagctatgaccggaccacctttctgaaggttc

SEQ ID NO: 549 - DNA, 39 bp, Homo sapiens; identical to SEQ ID NO: 548

SEQ ID NO: 550 - DNA, 39 bp, Homo sapiens; identical to SEQ ID NO: 548

SEQ ID NO: 551 - DNA, 39 bp, Homo sapiens; identical to SEQ ID NO: 548

SEQ ID NO: 552 - DNA, 39 bp, Homo sapiens
caggaaacagctatgacctgtcattttgtgcagcttctg

SEQ ID NO: 553 - DNA, 39 bp, Homo sapiens
caggaaacagctatgacctcaatcttctgcagtgtgtgc

SEQ ID NO: 554 - DNA, 40 bp, Homo sapiens
caggaaacagctatgacctgaccatacacttctcctgctt

SEQ ID NO: 555 - DNA, 40 bp, Homo sapiens; identical to SEQ ID NO: 554

SEQ ID NO: 556 - DNA, 39 bp, Homo sapiens
caggaaacagctatgaccctcctgagtgcaagtgattcc

SEQ ID NO: 557 - DNA, 39 bp, Homo sapiens; identical to SEQ ID NO: 556

SEQ ID NO: 558 - DNA, 39 bp, Homo sapiens
caggaaacagctatgaccacagctggttggtagcaggta

SEQ ID NO: 559 - DNA, 39 bp, Homo sapiens
caggaaacagctatgaccaattgtatgtgggggcagact

SEQ ID NO: 560 - DNA, 39 bp, Homo sapiens; identical to SEQ ID NO: 559

SEQ ID NO: 561 - DNA, 39 bp, Homo sapiens; identical to SEQ ID NO: 559

SEQ ID NO: 562 - DNA, 39 bp, Homo sapiens; identical to SEQ ID NO: 559

SEQ ID NO: 563 - DNA, 39 bp, Homo sapiens
caggaaacagctatgaccagatctctgggactttggagg

SEQ ID NO: 564 - DNA, 39 bp, Homo sapiens
caggaaacagctatgaccatttacttgtgggcttcctgg

SEQ ID NO: 565 - DNA, 39 bp, Homo sapiens; identical to SEQ ID NO: 564

SEQ ID NO: 566 - DNA, 39 bp, Homo sapiens
caggaaacagctatgaccgcattttctgaacctgcactc

SEQ ID NO: 567 - DNA, 39 bp, Homo sapiens
caggaaacagctatgaccgtcttttgttttggagggctc

SEQ ID NO: 568 - DNA, 39 bp, Homo sapiens
caggaaacagctatgaccatgcacataccacagaggagg

SEQ ID NO: 569 - DNA, 39 bp, Homo sapiens; identical to SEQ ID NO: 568

SEQ ID NO: 570 - DNA, 39 bp, Homo sapiens
caggaaacagctatgaccccagatgctgagacactagcc

SEQ ID NO: 571 - DNA, 39 bp, Homo sapiens
caggaaacagctatgacctgcacgctctcacctatacct

SEQ ID NO: 572 - DNA, 39 bp, Homo sapiens
caggaaacagctatgacccctccttctaccaaggtccat

SEQ ID NO: 573 - DNA, 39 bp, Homo sapiens
caggaaacagctatgacctaaacaagcatcccaggtgac

SEQ ID NO: 574 - DNA, 39 bp, Homo sapiens
caggaaacagctatgaccaggggcttcacctcactttag

SEQ ID NO: 575 - DNA, 39 bp, Homo sapiens
caggaaacagctatgacctgcaaaggtttctaggcaatg

SEQ ID NO: 576 - DNA, 39 bp, Homo sapiens
caggaaacagctatgaccccacctcaccctctcttcttc

SEQ ID NO: 577 - DNA, 39 bp, Homo sapiens
caggaaacagctatgaccttgtgtgtccttaggcaaagc

SEQ ID NO: 578 - DNA, 39 bp, Homo sapiens
caggaaacagctatgacctcctccttaagcacccaactt

SEQ ID NO: 579 - DNA, 39 bp, Homo sapiens
caggaaacagctatgaccgggactcacagatgacagcat

SEQ ID NO: 580 - DNA, 39 bp, Homo sapiens
caggaaacagctatgacctgaaagcatagactgcgtgtg

SEQ ID NO: 581 - DNA, 39 bp, Homo sapiens; identical to SEQ ID NO: 580

SEQ ID NO: 582 - DNA, 39 bp, Homo sapiens; identical to SEQ ID NO: 580

SEQ ID NO: 583 - DNA, 39 bp, Homo sapiens; identical to SEQ ID NO: 580

SEQ ID NO: 584 - DNA, 39 bp, Homo sapiens
caggaaacagctatgaccttttgctgcttgttctcaaca

SEQ ID NO: 585 - DNA, 39 bp, Homo sapiens
caggaaacagctatgaccttgggaagtttaccacactgc

SEQ ID NO: 586 - DNA, 36 bp, Homo sapiens
caggaaacagctatgaccgacttgtgttccccgcct

SEQ ID NO: 587 - DNA, 36 bp, Homo sapiens; identical to SEQ ID NO: 586

SEQ ID NO: 588 - DNA, 36 bp, Homo sapiens; identical to SEQ ID NO: 586

SEQ ID NO: 589 - DNA, 18 bp, Homo sapiens
tgtaaaacgacggccagt

SEQ ID NO: 590 - DNA, 18 bp, Homo sapiens
tgtaaaacgacggccagt

SEQ ID NO: 591 - DNA, 18 bp, Homo sapiens
tgtaaaacgacggccagt

SEQ ID NO: 592 - DNA, 18 bp, Homo sapiens
tgtaaaacgacggccagt

SEQ ID NO: 593 - DNA, 18 bp, Homo sapiens
tgtaaaacgacggccagt

SEQ ID NO: 594 - DNA, 18 bp, Homo sapiens
tgtaaaacgacggccagt

SEQ ID NO: 595 - DNA, 18 bp, Homo sapiens
tgtaaaacgacggccagt

SEQ ID NO: 596 - DNA, 18 bp, Homo sapiens
tgtaaaacgacggccagt

SEQ ID NO: 597 - DNA, 18 bp, Homo sapiens
tgtaaaacgacggccagt

SEQ ID NO: 598 - DNA, 18 bp, Homo sapiens
tgtaaaacgacggccagt

SEQ ID NO: 599 - DNA, 18 bp, Homo sapiens
tgtaaaacgacggccagt

SEQ ID NO: 600 - DNA, 18 bp, Homo sapiens
tgtaaaacgacggccagt

SEQ ID NO: 601 - DNA, 18 bp, Homo sapiens
tgtaaaacgacggccagt

SEQ ID NO: 602 - DNA, 18 bp, Homo sapiens
tgtaaaacgacggccagt

SEQ ID NO: 603 - DNA, 18 bp, Homo sapiens
tgtaaaacgacggccagt

SEQ ID NO: 604 - DNA, 18 bp, Homo sapiens
tgtaaaacgacggccagt

SEQ ID NO: 605 - DNA, 18 bp, Homo sapiens
tgtaaaacgacggccagt

SEQ ID NO: 606 - DNA, 18 bp, Homo sapiens
tgtaaaacgacggccagt

SEQ ID NO: 607 - DNA, 18 bp, Homo sapiens
tgtaaaacgacggccagt

SEQ ID NO: 608 - DNA, 18 bp, Homo sapiens
tgtaaaacgacggccagt

SEQ ID NO: 609 - DNA, 18 bp, Homo sapiens
tgtaaaacgacggccagt

SEQ ID NO: 610 - DNA, 18 bp, Homo sapiens
tgtaaaacgacggccagt

SEQ ID NO: 611 - DNA, 18 bp, Homo sapiens
tgtaaaacgacggccagt

SEQ ID NO: 612 - DNA, 18 bp, Homo sapiens
tgtaaaacgacggccagt

SEQ ID NO: 613 - DNA, 18 bp, Homo sapiens
tgtaaaacgacggccagt

SEQ ID NO: 614 - DNA, 18 bp, Homo sapiens
tgtaaaacgacggccagt

SEQ ID NO: 615 - DNA, 18 bp, Homo sapiens
tgtaaaacgacggccagt

SEQ ID NO: 616 - DNA, 18 bp, Homo sapiens
tgtaaaacgacggccagt

SEQ ID NO: 617 - DNA, 18 bp, Homo sapiens
tgtaaaacgacggccagt

SEQ ID NO: 618 - DNA, 18 bp, Homo sapiens
tgtaaaacgacggccagt

SEQ ID NO: 619 - DNA, 18 bp, Homo sapiens
tgtaaaacgacggccagt

SEQ ID NO: 620 - DNA, 18 bp, Homo sapiens
tgtaaaacgacggccagt

SEQ ID NO: 621 - DNA, 18 bp, Homo sapiens
tgtaaaacgacggccagt

SEQ ID NO: 622 - DNA, 18 bp, Homo sapiens
tgtaaaacgacggccagt

SEQ ID NO: 623 - DNA, 18 bp, Homo sapiens
tgtaaaacgacggccagt

SEQ ID NO: 624 - DNA, 18 bp, Homo sapiens
tgtaaaacgacggccagt

SEQ ID NO: 625 - DNA, 18 bp, Homo sapiens
tgtaaaacgacggccagt

SEQ ID NO: 626 - DNA, 18 bp, Homo sapiens
tgtaaaacgacggccagt

SEQ ID NO: 627 - DNA, 18 bp, Homo sapiens
tgtaaaacgacggccagt

SEQ ID NO: 628 - DNA, 18 bp, Homo sapiens
tgtaaaacgacggccagt

SEQ ID NO: 629 - DNA, 18 bp, Homo sapiens
tgtaaaacgacggccagt

SEQ ID NO: 630 - DNA, 18 bp, Homo sapiens
tgtaaaacgacggccagt

SEQ ID NO: 631 - DNA, 18 bp, Homo sapiens
tgtaaaacgacggccagt

SEQ ID NO: 632 - DNA, 18 bp, Homo sapiens
tgtaaaacgacggccagt

SEQ ID NO: 633 - DNA, 18 bp, Homo sapiens
tgtaaaacgacggccagt

SEQ ID NO: 634 - DNA, 18 bp, Homo sapiens
tgtaaaacgacggccagt

SEQ ID NO: 635 - DNA, 18 bp, Homo sapiens
tgtaaaacgacggccagt

SEQ ID NO: 636 - DNA, 18 bp, Homo sapiens
tgtaaaacgacggccagt

SEQ ID NO: 637 - DNA, 18 bp, Homo sapiens
tgtaaaacgacggccagt

SEQ ID NO: 638 - DNA, 18 bp, Homo sapiens
tgtaaaacgacggccagt

SEQ ID NO: 639 - DNA, 18 bp, Homo sapiens
tgtaaaacgacggccagt

SEQ ID NO: 640 - DNA, 18 bp, Homo sapiens
tgtaaaacgacggccagt

SEQ ID NO: 641 - DNA, 18 bp, Homo sapiens
tgtaaaacgacggccagt

SEQ ID NO: 642 - DNA, 18 bp, Homo sapiens
tgtaaaacgacggccagt

SEQ ID NO: 643 - DNA, 18 bp, Homo sapiens
tgtaaaacgacggccagt

SEQ ID NO: 644 - DNA, 18 bp, Homo sapiens
tgtaaaacgacggccagt

SEQ ID NO: 645 - DNA, 18 bp, Homo sapiens
tgtaaaacgacggccagt

SEQ ID NO: 646 - DNA, 18 bp, Homo sapiens
tgtaaaacgacggccagt

SEQ ID NO: 647 - DNA, 18 bp, Homo sapiens
tgtaaaacgacggccagt

SEQ ID NO: 648 - DNA, 18 bp, Homo sapiens
tgtaaaacgacggccagt

SEQ ID NO: 649 - DNA, 18 bp, Homo sapiens
tgtaaaacgacggccagt

SEQ ID NO: 650 - DNA, 18 bp, Homo sapiens
tgtaaaacgacggccagt

SEQ ID NO: 651 - DNA, 18 bp, Homo sapiens
tgtaaaacgacggccagt

SEQ ID NO: 652 - DNA, 18 bp, Homo sapiens
tgtaaaacgacggccagt

SEQ ID NO: 653 - DNA, 18 bp, Homo sapiens
tgtaaaacgacggccagt

SEQ ID NO: 654 - DNA, 18 bp, Homo sapiens
tgtaaaacgacggccagt

SEQ ID NO: 655 - DNA, 18 bp, Homo sapiens
tgtaaaacgacggccagt

SEQ ID NO: 656 - DNA, 18 bp, Homo sapiens
tgtaaaacgacggccagt

SEQ ID NO: 657 - DNA, 18 bp, Homo sapiens
tgtaaaacgacggccagt

SEQ ID NO: 658 - DNA, 18 bp, Homo sapiens
tgtaaaacgacggccagt

SEQ ID NO: 659 - DNA, 18 bp, Homo sapiens
tgtaaaacgacggccagt

SEQ ID NO: 660 - DNA, 18 bp, Homo sapiens
tgtaaaacgacggccagt

SEQ ID NO: 661 - DNA, 18 bp, Homo sapiens
tgtaaaacgacggccagt

SEQ ID NO: 662 - DNA, 18 bp, Homo sapiens
tgtaaaacgacggccagt

SEQ ID NO: 663 - DNA, 18 bp, Homo sapiens
tgtaaaacgacggccagt

SEQ ID NO: 664 - DNA, 18 bp, Homo sapiens
tgtaaaacgacggccagt

SEQ ID NO: 665 - DNA, 18 bp, Homo sapiens
tgtaaaacgacggccagt

SEQ ID NO: 666 - DNA, 18 bp, Homo sapiens
tgtaaaacgacggccagt

SEQ ID NO: 667 - DNA, 18 bp, Homo sapiens
tgtaaaacgacggccagt

SEQ ID NO: 668 - DNA, 18 bp, Homo sapiens
tgtaaaacgacggccagt

SEQ ID NO: 669 - DNA, 18 bp, Homo sapiens
tgtaaaacgacggccagt

SEQ ID NO: 670 - DNA, 18 bp, Homo sapiens
tgtaaaacgacggccagt

SEQ ID NO: 671 - DNA, 18 bp, Homo sapiens
tgtaaaacgacggccagt

SEQ ID NO: 672 - DNA, 18 bp, Homo sapiens
tgtaaaacgacggccagt

SEQ ID NO: 673 - DNA, 18 bp, Homo sapiens
tgtaaaacgacggccagt

SEQ ID NO: 674 - DNA, 18 bp, Homo sapiens
tgtaaaacgacggccagt

SEQ ID NO: 675 - DNA, 18 bp, Homo sapiens
tgtaaaacgacggccagt

SEQ ID NO: 676 - DNA, 18 bp, Homo sapiens
tgtaaaacgacggccagt

SEQ ID NO: 677 - DNA, 18 bp, Homo sapiens
tgtaaaacgacggccagt

SEQ ID NO: 678 - DNA, 18 bp, Homo sapiens
tgtaaaacgacggccagt

SEQ ID NO: 679 - DNA, 18 bp, Homo sapiens
tgtaaaacgacggccagt

SEQ ID NO: 680 - DNA, 18 bp, Homo sapiens
tgtaaaacgacggccagt

SEQ ID NO: 681 - DNA, 18 bp, Homo sapiens
tgtaaaacgacggccagt

SEQ ID NO: 682 - DNA, 18 bp, Homo sapiens
tgtaaaacgacggccagt

SEQ ID NO: 683 - DNA, 18 bp, Homo sapiens
tgtaaaacgacggccagt

SEQ ID NO: 684 - DNA, 18 bp, Homo sapiens
tgtaaaacgacggccagt

SEQ ID NO: 685 - DNA, 18 bp, Homo sapiens
tgtaaaacgacggccagt

SEQ ID NO: 686 - DNA, 18 bp, Homo sapiens
tgtaaaacgacggccagt

SEQ ID NO: 687 - DNA, 18 bp, Homo sapiens
tgtaaaacgacggccagt

SEQ ID NO: 688 - DNA, 18 bp, Homo sapiens
tgtaaaacgacggccagt

SEQ ID NO: 689 - DNA, 18 bp, Homo sapiens
tgtaaaacgacggccagt

SEQ ID NO: 690 - DNA, 18 bp, Homo sapiens
tgtaaaacgacggccagt

SEQ ID NO: 691 - DNA, 18 bp, Homo sapiens
tgtaaaacgacggccagt

SEQ ID NO: 692 - DNA, 18 bp, Homo sapiens
tgtaaaacgacggccagt

SEQ ID NO: 693 - DNA, 18 bp, Homo sapiens
tgtaaaacgacggccagt

SEQ ID NO: 694 - DNA, 18 bp, Homo sapiens
tgtaaaacgacggccagt

SEQ ID NO: 695 - DNA, 18 bp, Homo sapiens
tgtaaaacgacggccagt

SEQ ID NO: 696 - DNA, 18 bp, Homo sapiens
tgtaaaacgacggccagt

SEQ ID NO: 697 - DNA, 18 bp, Homo sapiens
tgtaaaacgacggccagt

SEQ ID NO: 698 - DNA, 18 bp, Homo sapiens
tgtaaaacgacggccagt

SEQ ID NO: 699 - DNA, 18 bp, Homo sapiens
tgtaaaacgacggccagt

SEQ ID NO: 700 - DNA, 18 bp, Homo sapiens
tgtaaaacgacggccagt

SEQ ID NO: 701 - DNA, 18 bp, Homo sapiens
tgtaaaacgacggccagt

SEQ ID NO: 702 - DNA, 18 bp, Homo sapiens
tgtaaaacgacggccagt

SEQ ID NO: 703 - DNA, 18 bp, Homo sapiens
tgtaaaacgacggccagt

SEQ ID NO: 704 - DNA, 18 bp, Homo sapiens
tgtaaaacgacggccagt

SEQ ID NO: 705 - DNA, 18 bp, Homo sapiens
tgtaaaacgacggccagt

SEQ ID NO: 706 - DNA, 18 bp, Homo sapiens
tgtaaaacgacggccagt

SEQ ID NO: 707 - DNA, 18 bp, Homo sapiens
tgtaaaacgacggccagt

SEQ ID NO: 708 - DNA, 18 bp, Homo sapiens
tgtaaaacgacggccagt

SEQ ID NO: 709 - DNA, 18 bp, Homo sapiens
tgtaaaacgacggccagt

SEQ ID NO: 710 - DNA, 18 bp, Homo sapiens
tgtaaaacgacggccagt

SEQ ID NO: 711 - DNA, 18 bp, Homo sapiens
tgtaaaacgacggccagt

SEQ ID NO: 712 - DNA, 18 bp, Homo sapiens
tgtaaaacgacggccagt

SEQ ID NO: 713 - DNA, 18 bp, Homo sapiens
tgtaaaacgacggccagt

SEQ ID NO: 714 - DNA, 18 bp, Homo sapiens
tgtaaaacgacggccagt

SEQ ID NO: 715 - DNA, 18 bp, Homo sapiens
tgtaaaacgacggccagt

SEQ ID NO: 716 - DNA, 18 bp, Homo sapiens
tgtaaaacgacggccagt

SEQ ID NO: 717 - DNA, 18 bp, Homo sapiens
tgtaaaacgacggccagt

SEQ ID NO: 718 - DNA, 18 bp, Homo sapiens
tgtaaaacgacggccagt

SEQ ID NO: 719 - DNA, 18 bp, Homo sapiens
tgtaaaacgacggccagt

SEQ ID NO: 720 - DNA, 18 bp, Homo sapiens
tgtaaaacgacggccagt

SEQ ID NO: 721 - DNA, 18 bp, Homo sapiens
tgtaaaacgacggccagt

SEQ ID NO: 722 - DNA, 18 bp, Homo sapiens
tgtaaaacgacggccagt

SEQ ID NO: 723 - DNA, 18 bp, Homo sapiens
tgtaaaacgacggccagt

SEQ ID NO: 724 - DNA, 18 bp, Homo sapiens
tgtaaaacgacggccagt

SEQ ID NO: 725 - DNA, 18 bp, Homo sapiens
tgtaaaacgacggccagt

SEQ ID NO: 726 - DNA, 18 bp, Homo sapiens
tgtaaaacgacggccagt

SEQ ID NO: 727 - DNA, 18 bp, Homo sapiens
tgtaaaacgacggccagt

SEQ ID NO: 728 - DNA, 18 bp, Homo sapiens
tgtaaaacgacggccagt

SEQ ID NO: 729 - DNA, 18 bp, Homo sapiens
tgtaaaacgacggccagt

SEQ ID NO: 730 - DNA, 18 bp, Homo sapiens
tgtaaaacgacggccagt

SEQ ID NO: 731 - DNA, 18 bp, Homo sapiens
tgtaaaacgacggccagt

SEQ ID NO: 732 - DNA, 18 bp, Homo sapiens
tgtaaaacgacggccagt

SEQ ID NO: 733 - DNA, 18 bp, Homo sapiens
tgtaaaacgacggccagt

SEQ ID NO: 734 - DNA, 18 bp, Homo sapiens
tgtaaaacgacggccagt

SEQ ID NO: 735 - DNA, 18 bp, Homo sapiens
tgtaaaacgacggccagt

SEQ ID NO: 736 - DNA, 18 bp, Homo sapiens
caggaaacagctatgacc

SEQ ID NO: 737 - DNA, 18 bp, Homo sapiens
caggaaacagctatgacc

SEQ ID NO: 738 - DNA, 18 bp, Homo sapiens
caggaaacagctatgacc

SEQ ID NO: 739 - DNA, 18 bp, Homo sapiens
caggaaacagctatgacc

SEQ ID NO: 740 - DNA, 18 bp, Homo sapiens
caggaaacagctatgacc

SEQ ID NO: 741 - DNA, 18 bp, Homo sapiens
caggaaacagctatgacc

SEQ ID NO: 742 - DNA, 18 bp, Homo sapiens
caggaaacagctatgacc

SEQ ID NO: 743 - DNA, 18 bp, Homo sapiens
caggaaacagctatgacc

SEQ ID NO: 744 - DNA, 18 bp, Homo sapiens
caggaaacagctatgacc

SEQ ID NO: 745 - DNA, 18 bp, Homo sapiens
caggaaacagctatgacc

SEQ ID NO: 746 - DNA, 18 bp, Homo sapiens
caggaaacagctatgacc

SEQ ID NO: 747 - DNA, 18 bp, Homo sapiens
caggaaacagctatgacc

SEQ ID NO: 748 - DNA, 18 bp, Homo sapiens
caggaaacagctatgacc

SEQ ID NO: 749 - DNA, 18 bp, Homo sapiens
caggaaacagctatgacc

SEQ ID NO: 750 - DNA, 18 bp, Homo sapiens
caggaaacagctatgacc

SEQ ID NO: 751 - DNA, 18 bp, Homo sapiens
caggaaacagctatgacc

SEQ ID NO: 752 - DNA, 18 bp, Homo sapiens
caggaaacagctatgacc

SEQ ID NO: 753 - DNA, 18 bp, Homo sapiens
caggaaacagctatgacc

SEQ ID NO: 754 - DNA, 18 bp, Homo sapiens
caggaaacagctatgacc

SEQ ID NO: 755 - DNA, 18 bp, Homo sapiens
caggaaacagctatgacc

SEQ ID NO: 756 - DNA, 18 bp, Homo sapiens
caggaaacagctatgacc

SEQ ID NO: 757 - DNA, 18 bp, Homo sapiens
caggaaacagctatgacc

SEQ ID NO: 758 - DNA, 18 bp, Homo sapiens
caggaaacagctatgacc

SEQ ID NO: 759 - DNA, 18 bp, Homo sapiens
caggaaacagctatgacc

SEQ ID NO: 760 - DNA, 18 bp, Homo sapiens
caggaaacagctatgacc

SEQ ID NO: 761 - DNA, 18 bp, Homo sapiens
caggaaacagctatgacc

SEQ ID NO: 762 - DNA, 18 bp, Homo sapiens
caggaaacagctatgacc

SEQ ID NO: 763 - DNA, 18 bp, Homo sapiens
caggaaacagctatgacc

SEQ ID NO: 764 - DNA, 18 bp, Homo sapiens
caggaaacagctatgacc

SEQ ID NO: 765 - DNA, 18 bp, Homo sapiens
caggaaacagctatgacc

SEQ ID NO: 766 - DNA, 18 bp, Homo sapiens
caggaaacagctatgacc

SEQ ID NO: 767 - DNA, 18 bp, Homo sapiens
caggaaacagctatgacc

SEQ ID NO: 768 - DNA, 18 bp, Homo sapiens
caggaaacagctatgacc

SEQ ID NO: 769 - DNA, 18 bp, Homo sapiens
caggaaacagctatgacc

SEQ ID NO: 770 - DNA, 18 bp, Homo sapiens
caggaaacagctatgacc

SEQ ID NO: 771 - DNA, 18 bp, Homo sapiens
caggaaacagctatgacc

SEQ ID NO: 772 - DNA, 18 bp, Homo sapiens
caggaaacagctatgacc

SEQ ID NO: 773 - DNA, 18 bp, Homo sapiens
caggaaacagctatgacc

SEQ ID NO: 774 - DNA, 18 bp, Homo sapiens
caggaaacagctatgacc

SEQ ID NO: 775 - DNA, 18 bp, Homo sapiens
caggaaacagctatgacc

SEQ ID NO: 776 - DNA, 18 bp, Homo sapiens
caggaaacagctatgacc

SEQ ID NO: 777 - DNA, 18 bp, Homo sapiens
caggaaacagctatgacc

SEQ ID NO: 778 - DNA, 18 bp, Homo sapiens
caggaaacagctatgacc

SEQ ID NO: 779 - DNA, 18 bp, Homo sapiens
caggaaacagctatgacc

SEQ ID NO: 780 - DNA, 18 bp, Homo sapiens
caggaaacagctatgacc

SEQ ID NO: 781 - DNA, 18 bp, Homo sapiens
caggaaacagctatgacc

SEQ ID NO: 782 - DNA, 18 bp, Homo sapiens
caggaaacagctatgacc

SEQ ID NO: 783 - DNA, 18 bp, Homo sapiens
caggaaacagctatgacc

SEQ ID NO: 784 - DNA, 18 bp, Homo sapiens
caggaaacagctatgacc

SEQ ID NO: 785 - DNA, 18 bp, Homo sapiens
caggaaacagctatgacc

SEQ ID NO: 786 - DNA, 18 bp, Homo sapiens
caggaaacagctatgacc

SEQ ID NO: 787 - DNA, 18 bp, Homo sapiens
caggaaacagctatgacc

SEQ ID NO: 788 - DNA, 18 bp, Homo sapiens
caggaaacagctatgacc

SEQ ID NO: 789 - DNA, 18 bp, Homo sapiens
caggaaacagctatgacc

SEQ ID NO: 790 - DNA, 18 bp, Homo sapiens
caggaaacagctatgacc

SEQ ID NO: 791 - DNA, 18 bp, Homo sapiens
caggaaacagctatgacc

SEQ ID NO: 792 - DNA, 18 bp, Homo sapiens
caggaaacagctatgacc

SEQ ID NO: 793 - DNA, 18 bp, Homo sapiens
caggaaacagctatgacc

SEQ ID NO: 794 - DNA, 18 bp, Homo sapiens
caggaaacagctatgacc

SEQ ID NO: 795 - DNA, 18 bp, Homo sapiens
caggaaacagctatgacc

SEQ ID NO: 796 - DNA, 18 bp, Homo sapiens
caggaaacagctatgacc

SEQ ID NO: 797 - DNA, 18 bp, Homo sapiens
caggaaacagctatgacc

SEQ ID NO: 798 - DNA, 18 bp, Homo sapiens
caggaaacagctatgacc

SEQ ID NO: 799 - DNA, 18 bp, Homo sapiens
caggaaacagctatgacc

SEQ ID NO: 800 - DNA, 18 bp, Homo sapiens
caggaaacagctatgacc

SEQ ID NO: 801 - DNA, 18 bp, Homo sapiens
caggaaacagctatgacc

SEQ ID NO: 802 - DNA, 18 bp, Homo sapiens
caggaaacagctatgacc

SEQ ID NO: 803 - DNA, 18 bp, Homo sapiens
caggaaacagctatgacc

SEQ ID NO: 804 - DNA, 18 bp, Homo sapiens
caggaaacagctatgacc

SEQ ID NO: 805 - DNA, 18 bp, Homo sapiens
caggaaacagctatgacc

SEQ ID NO: 806 - DNA, 18 bp, Homo sapiens
caggaaacagctatgacc

SEQ ID NO: 807 - DNA, 18 bp, Homo sapiens
caggaaacagctatgacc

SEQ ID NO: 808 - DNA, 18 bp, Homo sapiens
caggaaacagctatgacc

SEQ ID NO: 809 - DNA, 18 bp, Homo sapiens
caggaaacagctatgacc

SEQ ID NO: 810 - DNA, 18 bp, Homo sapiens
caggaaacagctatgacc

SEQ ID NO: 811 - DNA, 18 bp, Homo sapiens
caggaaacagctatgacc

SEQ ID NO: 812 - DNA, 18 bp, Homo sapiens
caggaaacagctatgacc

SEQ ID NO: 813 - DNA, 18 bp, Homo sapiens
caggaaacagctatgacc

SEQ ID NO: 814 - DNA, 18 bp, Homo sapiens
caggaaacagctatgacc

SEQ ID NO: 815 - DNA, 18 bp, Homo sapiens
caggaaacagctatgacc

SEQ ID NO: 816 - DNA, 18 bp, Homo sapiens
caggaaacagctatgacc

SEQ ID NO: 817 - DNA, 18 bp, Homo sapiens
caggaaacagctatgacc

SEQ ID NO: 818 - DNA, 18 bp, Homo sapiens
caggaaacagctatgacc

SEQ ID NO: 819 - DNA, 18 bp, Homo sapiens
caggaaacagctatgacc

SEQ ID NO: 820 - DNA, 18 bp, Homo sapiens
caggaaacagctatgacc

SEQ ID NO: 821 - DNA, 18 bp, Homo sapiens
caggaaacagctatgacc

SEQ ID NO: 822 - DNA, 18 bp, Homo sapiens
caggaaacagctatgacc

SEQ ID NO: 823 - DNA, 18 bp, Homo sapiens
caggaaacagctatgacc

SEQ ID NO: 824 - DNA, 18 bp, Homo sapiens
caggaaacagctatgacc

SEQ ID NO: 825 - DNA, 18 bp, Homo sapiens
caggaaacagctatgacc

SEQ ID NO: 826 - DNA, 18 bp, Homo sapiens
caggaaacagctatgacc

SEQ ID NO: 827 - DNA, 18 bp, Homo sapiens
caggaaacagctatgacc

SEQ ID NO: 828 - DNA, 18 bp, Homo sapiens
caggaaacagctatgacc

SEQ ID NO: 829 - DNA, 18 bp, Homo sapiens
caggaaacagctatgacc

SEQ ID NO: 830 - DNA, 18 bp, Homo sapiens
caggaaacagctatgacc

SEQ ID NO: 831 - DNA, 18 bp, Homo sapiens
caggaaacagctatgacc

SEQ ID NO: 832 - DNA, 18 bp, Homo sapiens
caggaaacagctatgacc

SEQ ID NO: 833 - DNA, 18 bp, Homo sapiens
caggaaacagctatgacc

SEQ ID NO: 834 - DNA, 18 bp, Homo sapiens
caggaaacagctatgacc

SEQ ID NO: 835 - DNA, 18 bp, Homo sapiens
caggaaacagctatgacc

SEQ ID NO: 836 - DNA, 18 bp, Homo sapiens
caggaaacagctatgacc

SEQ ID NO: 837 - DNA, 18 bp, Homo sapiens
caggaaacagctatgacc

SEQ ID NO: 838 - DNA, 18 bp, Homo sapiens
caggaaacagctatgacc

SEQ ID NO: 839 - DNA, 18 bp, Homo sapiens
caggaaacagctatgacc

SEQ ID NO: 840 - DNA, 18 bp, Homo sapiens
caggaaacagctatgacc

SEQ ID NO: 841 - DNA, 18 bp, Homo sapiens
caggaaacagctatgacc

SEQ ID NO: 842 - DNA, 18 bp, Homo sapiens
caggaaacagctatgacc

SEQ ID NO: 843 - DNA, 18 bp, Homo sapiens
caggaaacagctatgacc

SEQ ID NO: 844 - DNA, 18 bp, Homo sapiens
caggaaacagctatgacc

SEQ ID NO: 845 - DNA, 18 bp, Homo sapiens
caggaaacagctatgacc

SEQ ID NO: 846 - DNA, 18 bp, Homo sapiens
caggaaacagctatgacc

SEQ ID NO: 847 - DNA, 18 bp, Homo sapiens
caggaaacagctatgacc

SEQ ID NO: 848 - DNA, 18 bp, Homo sapiens
caggaaacagctatgacc

SEQ ID NO: 849 - DNA, 18 bp, Homo sapiens
caggaaacagctatgacc

SEQ ID NO: 850 - DNA, 18 bp, Homo sapiens
caggaaacagctatgacc

SEQ ID NO: 851 - DNA, 18 bp, Homo sapiens
caggaaacagctatgacc

SEQ ID NO: 852 - DNA, 18 bp, Homo sapiens
caggaaacagctatgacc

SEQ ID NO: 853 - DNA, 18 bp, Homo sapiens
caggaaacagctatgacc

SEQ ID NO: 854 - DNA, 18 bp, Homo sapiens
caggaaacagctatgacc

SEQ ID NO: 855 - DNA, 18 bp, Homo sapiens
caggaaacagctatgacc

SEQ ID NO: 856 - DNA, 18 bp, Homo sapiens
caggaaacagctatgacc

SEQ ID NO: 857 - DNA, 18 bp, Homo sapiens
caggaaacagctatgacc

SEQ ID NO: 858 - DNA, 18 bp, Homo sapiens
caggaaacagctatgacc

SEQ ID NO: 859 - DNA, 18 bp, Homo sapiens
caggaaacagctatgacc

SEQ ID NO: 860 - DNA, 18 bp, Homo sapiens
caggaaacagctatgacc

SEQ ID NO: 861 - DNA, 18 bp, Homo sapiens
caggaaacagctatgacc

SEQ ID NO: 862 - DNA, 18 bp, Homo sapiens
caggaaacagctatgacc

SEQ ID NO: 863 - DNA, 18 bp, Homo sapiens
caggaaacagctatgacc

SEQ ID NO: 864 - DNA, 18 bp, Homo sapiens
caggaaacagctatgacc

SEQ ID NO: 865 - DNA, 18 bp, Homo sapiens
caggaaacagctatgacc

SEQ ID NO: 866 - DNA, 18 bp, Homo sapiens
caggaaacagctatgacc

SEQ ID NO: 867 - DNA, 18 bp, Homo sapiens
caggaaacagctatgacc

SEQ ID NO: 868 - DNA, 18 bp, Homo sapiens
caggaaacagctatgacc

SEQ ID NO: 869 - DNA, 18 bp, Homo sapiens
caggaaacagctatgacc

SEQ ID NO: 870 - DNA, 18 bp, Homo sapiens
caggaaacagctatgacc

SEQ ID NO: 871 - DNA, 18 bp, Homo sapiens
caggaaacagctatgacc

SEQ ID NO: 872 - DNA, 18 bp, Homo sapiens
caggaaacagctatgacc

SEQ ID NO: 873 - DNA, 18 bp, Homo sapiens
caggaaacagctatgacc

SEQ ID NO: 874 - DNA, 18 bp, Homo sapiens
caggaaacagctatgacc

SEQ ID NO: 875 - DNA, 18 bp, Homo sapiens
caggaaacagctatgacc

SEQ ID NO: 876 - DNA, 18 bp, Homo sapiens
caggaaacagctatgacc

SEQ ID NO: 877 - DNA, 18 bp, Homo sapiens
caggaaacagctatgacc

SEQ ID NO: 878 - DNA, 18 bp, Homo sapiens
caggaaacagctatgacc

SEQ ID NO: 879 - DNA, 18 bp, Homo sapiens
caggaaacagctatgacc

SEQ ID NO: 880 - DNA, 18 bp, Homo sapiens
caggaaacagctatgacc

SEQ ID NO: 881 - DNA, 18 bp, Homo sapiens
caggaaacagctatgacc

SEQ ID NO: 882 - DNA, 18 bp, Homo sapiens
caggaaacagctatgacc

SEQ ID NO: 883 - DNA, 2647 bp, Homo sapiens
  misc_feature (558)..(558): wherein “n” equals either ′G′ or ′A′.
gggccggagttcctgcagagggagcgtcaaggccctgtgctgctgtccctgggggccagaggggttgcccagcatgcccactggcaggagagagggaactgacccacttgctcctaccagcttctgaaggctccaaagtccggagtgcagaaagccaggaccaagagacaggcagctcaccagggtggacaaatcgccagagatgtggtgcattgtcctgttttcacttttggcatgggtttatgctgagcctaccatgtatggggagatcctgtcccctaactatcctcaggcatatcccagtgaggtagagaaatcttgggacatagaagttcctgaagggtatgggattcacctctacttcacccatctggacattgagctgtcagagaactgtgcgtatgactcagtgcagataatctcaggagacactgaagaagggaggctctgtggacagaggagcagtaacaatccccactctccaattgtggaagagttccaagtcccatacaacaaactccaggtgatctttaagtcagacttttccaatgaagagcnttttacggggtttgctgcatactatgttgccacagacataaatgaatgcacagattttgtagatgtcccttgtagccacttctgnaacaatttcattggtggttacttctgctcctgccccccggaatatttcctccatgatgacatgaagaattgcggagttaattgcagtggggatgtattcactgcactgattggggagattgcaagtcccaattatcccaaaccatatccagagaactcaaggtgtgaataccagatccggttggagaaagggttccaagtggtggtgaccttgcggagagaagattttgatgtggaagcagctgactcagcgggaaactgccttgacagtttagtttttgttgcaggagatcggcaatttggtccttactgtggtcatggattccctgggcctctaaanattgaaaccaagagtaatgctcttgatatcatcttccaaactgatctaacagggcaaaaaaagggctggaaacttcgctatcatggagatccaatgccctgccctaaggaagacactcccaattctgtttgggagcctgngaaggcaaaatatntctttagagatgtggtgcagataacctgtctggatgggtttgaagttgtggagggacgtgttggtgcaacatctttctattcgacttgtcaaagcaatggaaagtggagtaattccaaactgaaatgtcaacctgtggactgtggcattcctgaatccattgagaatggtaaagttgaagacccagagagcactttgtttggttctgtcatccgctacacttgtgaggagccatattactacatggaaaatggaggaggtggggagtatcactgtgctggtaacgggagctgggtgaatgaggtgctgggcccggagctgccgaaatgtgttccagtctgtggagtccccagagaaccctttgaagaaaaacagaggataattggaggatccgatgcagatattaaaaacttcccctggcaagtcttctttgacaacccatgggctggtggagcgctcattaatgagtactgggtgctgacggctgctcatgttgtggagggaaacagggagccaacaatgtatgttgggtccacctcagtgcagacctcacggctggcaaaatccaagatgctcactcctgagcatgtgtttattcatccgggatggaagctgctggaagtcccagaaggacgaaccaattttgataatgacattgcactggtgcggctgaaagacccagtgaaaatgggacccaccgtctctcccatctgcctaccaggcacctcttccgactacaacctnatggatggggacctgggactgatctcaggctggggccgaacagagaagagagatcgtgctgttcgcctcaaggcggcaaggttacctgtagctcctttaagaaaatgcaaagaagtgaaagtggagaaacccacagcagatgcagaggcctatgttttcactcctaacatgatctgtgctggaggagagaagggcatggatagctgtaaaggggacagtggtggggcctttgctgtacaggatcccaatgacaagaccaaattctacgcagctggcctggtgtcctgggggccccagtgtgggacctatgggctctacacacgggtaaagaactatgttgactggataatgaagactatgcaggaaaatagcaccccccgtgaggactaatccagatacatcccaccagcctctccaagggtggtgaccaatgcattaccttctgttccttatgatattctcattatttcatcatgactgaaagaagacacgagcgaatgatttaaatagaacttgattgttgagacgccttgctagaggtagagtttgatcatagaattgtgctggtcatacatttgtggtctgactccttggggtcctttccccggagtacctattgtagataacactatgggtggggcactcctttcttgcactattccacagggataccttaattctttgtttcctctttacctgttcaaaattccatttacttgatcattctcagtatccactgtctatgtacaataaaggatgtttataagc

SEQ ID NO: 884 - protein, 688 aa, Homo sapiens
  MISC_FEATURE (119)..(119): wherein “X” equals ′Arg′ or ′His′.
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEEXFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLXIETKSNALDIIFQTDLTGQKKGWKLRYHGDPMPCPKEDTPNSVWEPXKAKYXFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 885 - DNA, 3494 bp, Homo sapiens
  misc_feature (474)..(474): wherein “n” equals either ′C′ or ′T′.
taatttttgcccagtctgcctgttgtggggctcctcccctttggggatataagcccggcctggggctgctccgttctctgcctggcctgaggctccctgagccgcctccccaccatcaccatggccaagggcttctatatttccaagtccctgggcatcctggggatcctcctgggcgtggcagccgtgtgcacaatcatcgcactgtcagtggtgtactcccaggagaagaacaagaacgccaacagctcccccgtggcctccaccaccccgtccgcctcagccaccaccaaccccgcctcggccaccaccttggaccaaagtaaagcgtggaatcgttaccgcctccccaacacgctgaaacccgattcctaccaggtgacgctgagaccgtacctcacccccaatgacaggggcctgtacgtttttaagggctccagcaccgtccgtttcacctgcaaggaggccactgangtcatcatcatccacagcaagaagctcaactacaccctcagccaggggcacagggtggtcctgcgtggtgtgggaggctcccagccccccgacattgacaagactgagctggtggagcccaccgagtacctggtggtgcacctcaagggctccctggtgaaggacagccagtatgagatggacagcgagttcgagggggagttggcagatgacctggcgggcttctaccgcagcgagtacatggagggcaatgtcagaaaggtggtggccacnacacagatgcaggctgcagatgcccggaagtccttcccatgcttcgatgagccggccatgaaggccgagttcaacatcacgcttatccaccccaaggacctgacagccctgtccaacatgcttcccaaaggtcccagcaccccacttccagaagaccccaactggaatgtcactgagttccacaccacgcccaagatgtccacgtacttgctggccttcattgtcagtgagttcgactacgtggagaagcaggcatccaatggtgtcttgatccggatctgggcccggcccagtgccattgcggngggccacggcgattatgccctnaacgtgacnggccccatccttaacttctttgctggtcattatgacacaccctacccactcccaaaatcagaccagattggcctgccagacttcaacgccggcgccatggagaactggggactggtgacctaccgggagaactccctgctgttngaccccctgtcctcctccagcagcaacaaggagcgggtggtcactgtgattgctcatgagctggcccaccagtggttcgggaacctggtgaccatagagtggtggaatgacctgtggctgaacgagggcttcgcctcctacgtggagtacctgggtgctgactatgcggagcccacctggaacttgaaagacctcatggtgctgaatgatgtgtaccgcgtgatggcagtggatgcactggcctcctcccacccgctgtccacacccgcctcggagatcaacacgccggcccagatcagtgagctgtttgacgccatctcctacagcaagggcgcctcagtcctcaggatgctctccagcttcctgtccgaggacgtattcaagcagggcctggcgtcctacctccacacctttgcctaccagaacaccatctacctgaacctgtgggaccacctgcaggaggctgtgaacaaccggtccatccaactccccaccaccgtgcgggacatcatgaaccgctggaccctgcagatgggcttcccggtcatcacggtggataccagcacggggaccctttcccaggagcacttcctccttgaccccgattccaatgttacccgcccctcagaattcaactacgtgtggattgtgcccatcacatccatcagagatggcagacagcagcaggactactggctganngatgtaagagcccagaacgatctcttcagcacatcaggcaatgagtgggtcctgctgaacctcaatgtgacgggctattaccgggtgaactacgacgaagagaactggaggaagattcagactcagctgcagagagaccactcggccatccctgtcatcaatcgggcacagatcattaatgacgccttcaacctggccagtgcccataaggtccctgtcactctggcgctgaacaacaccctcttcctgattgaagagagacagtacatgccctgggaggccgccctgagcagcctgagctacttcaagctcatgtttgaccgctccgaggtctatggccccatgaagaactacctgaagaagcaggtcacacccctcttcattcacttcagaaataataccaacaactggagggagatcccagaaaacctgatggaccagtacagcgaggttaatgccatcagcaccgcctgctccaacggagttccagagtgtgaggagatggtctctggccttttcaagcagtggatggagaaccccaataataacccgatccaccccaacctgcggtccacngtctactgcaacgntatcgcccagggcggggaggaggagtgggacttngcctgggagcagttccgaaatgccacactggtcaatgaggctgacaagctccgggcagccctggcctgcagcaaagagttgtggatcctgaacaggtacctgagctacacnctgaacccggacttaatccggaagcaggacgccacctctaccatcatcagcattaccaacaacgtcatngggcaaggtctggtctgggactttgtccagagcaactggaagaagctttttaacgattatggtggtggctcgttctccttctccaacctcatccaggcagtgacacgacgattctccaccgagtatgagctgcagcagctggagcagttcaagaaggacaacgaggaaacaggcttcggctcaggcacccgggccctggagcaagccctggagaagacgaaagccaacatcaagtgggtgaaggagaacaaggaggtggtgctccagtggttcacagaaaacagcaaatagtccccagcccttgaagtcacccggccccgatgcaaggtgcccacatgtgtccatcccagcggctggtgcagggcctccattcctggagcccgaggcaccagtgtcctcccctcaaggacaaagtctccagcccacgttctctctgcctgtgagccagtctagttcctgatgacccaggctgcctgagcacctcccagcccctgcccctcatgccaaccccgccctaggcctggcatggcacctgtcgcccagtgccctggggctgatctcagggaagcccagctccagggccagatgagcagaagctctcgatggacaatgaacggccttgctgggggccgccctgtaccctctttcacctttccctaaagaccctaaatctgaggaatcaacagggcagcagatctgtatatttttttctaagagaaaatgtaaataaaggatttctagatgaaaaaaaaaaaaaaaaa

SEQ ID NO: 886 - protein, 1027 aa, Homo sapiens
  MISC_FEATURE (311)..(311): wherein “X” equals ′Ala′ or ′Val′.
MAKGFYISKSLGILGILLGVAAVCTIIALSVVYSQEKNKNANSSPVASTTPSASATTNPASATTLDQSKAWNRYRLPNTLKPDSYQVTLRPYLTPNDRGLYVFKGSSTVRFTCKEATDVIIIHSKKLNYTLSQGHRVVLRGVGGSQPPDIDKTELVEPTEYLVVHLKGSLVKDSQYEMDSEFEGELADDLAGFYRSEYMEGNVRKVVATTQMQAADARKSFPCFDEPAMKAEFNITLIHPKDLTALSNMLPKGPSTPLPEDPNWNVTEFHTTPKMSTYLLAFIVSEFDYVEKQASNGVLIRIWARPSAIAXGHGDYALNVTGPILNFFAGHYDTPYPLPKSDQIGLPDFNAGAMENWGLVTYRENSLLFDPLSSSSSNKERVVTVIAHELAHQWFGNLVTIEWWNDLWLNEGFASYVEYLGADYAEPTWNLKDLMVLNDVYRVMAVDALASSHPLSTPASEINTPAQISELFDAISYSKGASVLRMLSSFLSEDVFKQGLASYLHTFAYQNTIYLNLWDHLQEAVNNRSIQLPTTVRDIMNRWTLQMGFPVITVDTSTGTLSQEHFLLDPDSNVTRPSEFNYVWIVPITSIRDGRQQQDYWLXDVRAQNDLFSTSGNEWVLLNLNVTGYYRVNYDEENWRKIQTQLQRDHSAIPVINRAQIINDAFNLASAHKVPVTLALNNTLFLIEERQYMPWEAALSSLSYFKLMFDRSEVYGPMKNYLKKQVTPLFIHFRNNTNNWREIPENLMDQYSEVNAISTACSNGVPECEEMVSGLFKQWMENPNNNPIHPNLRSTVYCNXIAQGGEEEWDFAWEQFRNATLVNEADKLRAALACSKELWILNRYLSYTLNPDLIRKQDATTCTACCATCATCAGCATTACCAACAACGTCATNGGGCAAGGTCTGGTCTGGGACTTTGTCSTIISITNNVIGQGLVWDFVQSNWKKLFNDYGGGSFSFSNLIQAVTRRFSTEYELQQLEQFKKDNEETGFGSGTRALEQALEKTKANIKWVKENKEVVLQWFTENSK

SEQ ID NO: 887 - DNA, 2326 bp, Homo sapiens
  misc_feature (196)..(196): wherein “n” equals either ′G′ or ′A′.
caaccctgaatgtcatagttagctactttcaactggaagctacaacatggatttatggaatctgtcttggtttctgttcttggatgctcttctcgtgatttctggcttggcaactccagaaaactttgatgtagatggcggaatggaccaggacatatttgatatcaatgaaggtttgggactggatctttttganggtgacatcagacttgatagggcacaaattagaaattccatcattggagaaaagtatagatggcctcataccattccatatgttctagaagatagcttggaaatgaatgctaagggagttatcctcaatgcatttgaacgttatcgccttaaaacatgtattgactttaagccttgggctggagaaacaaactatatntcagtgttcaagggcagtggctgctggtcttcagtaggaaataggcgggttgggaagcaagaactttccatcggggcaaactgtgaccgaatagcaacagttcaacacgagttcctccacgctctgggattctggcatgagcagtcgcgttctgaccgggatgactatgtcaggataatgtgggacagaattctgtcaggcagagagcacaattttaacacctatagtgacgatatatcagattccctgaatgttccctatgattacacttcagtaatgcactacagtaaaactgcattccaaaatggaacagagccgacaattgtcacaagaatctcagactttgaggatgtgatcggccaacgaatggatttcagtgactctgatctcctaaagttgaatcaactgtataactgctcctcttccttgagttttatggactcntgcagttttgaactggaaaatgtgtgtggcatgatccaaagttcaggagataatgctgactggcaacgggtttcacaggttcccagggggccagagagtgatcactccaacatgggccagtgccaaggttctggtttcttcatgcatttcgatagcagctctgtaaatgtgggggccacagcantgctggaaagtagaacgctgtaccctaaaagaggatttcagtgcctgcaattttacttatataacagtggcagtgaaagtgatcaactgaacatctatatcagggagtattctgcagacaatgtggatggcaatttaacccttgtggaagaaataaaagaaatacccactgggagctggcaactttatcatgtaacattgaaagtgaccaagaagtttagagtggtgtttgaaggacgcaaaggcnctggtgcatcactgggtggtctgtctattgatgacatcaatctttcngaaacacggtgccctcatcatatctggcatataaggaatttcacacagttcattggcagcccaaatggaactctgtatagccctccattttactcttctaaaggttatgcctttcagatttacttaaatctagcccatgtgactaatgcagggatatatttccacttgatctctggagccaatgatgatcaattacagtggccatgtccttggcaacaagccacaatgacacttttggatcaaaatcctgacattcgacagcgtatgtccaatcagcggagtataactacagacccatttatgaccaccgataatggaaactatttctgggacaggccttctaaagtgggaacagtggctttgttctctaatggaactcagtttagaagaggtgggggctatggaaccagtgcctttataacccnngaaaggctgaaaagcagagattttataaaaggagatgatgtttatatcctactgacagtggaagacatatctcacctcaactctacacaaatccagctaacaccagcccctagtgttcaagacctctgctcaaaaaccacctgtaaaaatgacggtgtctgcactgttcgagatggcaaagctgagtgcaggtgccagtcaggggaagactggtggtacatgggagaaaggtgtgaaaagagaggctccacccgaganaccatagtcattgctgtttcatctactgttgctgtgtttgccttgatgctgatcatcacccttgtcagtgtctattgcaccaggaagaaatatcgtgaaaggatgagctcaaatcgaccaaatttgactcngcaaaatcatgctttttgaagattaactcgacaatatggccagctaatgaaattaaaaaggattcttcatcatggatttcgcctaagtgatattacagccacctcattcttctaaaagtggatatttttctgtaaatagctggaaatattataaatcactattttggtcaaagtcaaaaaaaaaaaaaaaaaaaaa

SEQ ID NO: 888 - protein, 700 aa, Homo sapiens
  MISC_FEATURE (115)..(115): wherein “X” equals ′Ile′ or ′Met′.
MDLWNLSWFLFLDALLVISGLATPENFDVDGGMDQDIFDINEGLGLDLFEGDIRLDRAQIRNSIIGEKYRWPHTIPYVLEDSLEMNAKGVILNAFERYRLKTCIDFKPWAGETNYXSVFKGSGCWSSVGNRRVGKQELSIGANCDRIATVQHEFLHALGFWHEQSRSDRDDYVRIMWDRILSGREHNFNTYSDDISDSLNVPYDYTSVMHYSKTAFQNGTEPTIVTRISDFEDVIGQRMDFSDSDLLKLNQLYNCSSSLSFMDSCSFELENVCGMIQSSGDNADWQRVSQVPRGPESDHSNMGQCQGSGFFMHFDSSSVNVGATAXLESRTLYPKRGFQCLQFYLYNSGSESDQLNIYIREYSADNVDGNLTLVEEIKEIPTGSWQLYHVTLKVTKKFRVVFEGRKGXGASLGGLSIDDINLSETRCPHHIWHIRNFTQFIGSPNGTLYSPPFYSSKGYAFQIYLNLAHVTNAGIYFHLISGANDDQLQWPCPWQQATMTLLDQNPDIRQRMSNQRSITTDPFMTTDNGNYFWDRPSKVGTVALFSNGTQFRRGGGYGTSAFITXERLKSRDFIKGDDVYILLTVEDISHLNSTQIQLTPAPSVQDLCSKTTCKNDGVCTVRDGKAECRCQSGEDWWYMGERCEKRGSTRDTIVIAVSSTVAVFALMLIITLVSVYCTRKKYRERMSSNRPNLTXQNHAF

SEQ ID NO: 889 - DNA, 1872 bp, Homo sapiens
  misc_feature (225)..(225): wherein “n” equals either ′C′ or ′T′.
atgcctccaaaggtgacttcagagctgcttcggcagctgagacaagccatgaggaactctgagtatgtgaccgaaccgatccaggcctacatcatcccatcgggagatgctcatcagagtgagtatattgctccatgtgactgtcggcgggcttttgtctctggattcgatggctctgcgggcacagccatcatcacagaagagcatgcagccatgtggactgangggcgctactttctccaggctgccaagcaaatggacagcaactggacacttatgaagatgggtctgaaggacacaccaactcaggaagactggctggtgagtgtgcttcctgaaggatccagggttggtgtggaccccttgatcattcctacagattattggaagaaaatggccaaagttctgagaagtgccggccatcacctcattcctgtcaaggagaacctcgttgacaaaatctggacagaccgtcctgagcgcccttgcaagcctctcctcacactgggcctggattacacaggcatctcctggaaggacaaggttgcagaccttcggttgaaaatggctgagaggaacgtcatgtggtttgtggtcactgccttggatgagattgcgtggctatttaatctccgaggatcagatgtggagcacaatccagtatttttctcctacgcaatcataggactagagacgatcatgctcttcattgatggtgaccgcatagacgcccccagtgtgaaggagcacctgcttcttgacttgggtctggaagccgaatacaggatccaggtgcatccctacaagtccatcctgagcgagctcaaggccctgtgtgctgacctctccccaagggagaaggtgtgggtcagtgacaaggccagctatgctgtgagcgagaccatccccaaggaccaccgctgctgtatgccttacacccccatctgcatcgccaaagctgtgaagaattcagctgagtcagaaggcatgaggccggctcacattaaagatgctgttgctctctgtgaactctttaactggctggagaaagaggttcccaaaggtggtgtgacagagatctcagctgctgacaaagctgaggagtttcgcaggcaacaggcagactttgtggacctgagcttcccaacaatttccagtacgggacccaacggcgccatcattcactacgcgccagtccctgagacgaataggaccttgtccctggatgaggtgtaccttattgactcgggtgctcaatacaaggatggcaccacagatgtgacgcggacaatgcattttgggacccctacagcctacgagaaggaatgcttcacatatgtcctcaagggccacatagctgtgagtgcagccgttttcccgactggaaccaaaggtcaccttcttgactcctttgcccgttcagctttatgggattcaggcctagattacttgcacgggactggacatggtgttgggtcttttttgaatgtccatgagggtccttgcggcatcagttacaaaacattctctgatgagcccttggaggcaggcatgattgtcactgatgagcccgggtactatgaagatggggcttttggaattcgcattgagaatgttgtccttgtggttcctgtgaagaccaagtataattttaataaccggggaagcctgacctttgaacctctaacattggttccaattcagaccaaaatgatagatgtggattctcttacagacaaagagtgcgactggctcaacaattaccacctgacntgcagggatgtgattgggaaggaattgcagaaacagggccgccaggaagctctcgagtggctcatcagagagacgcaacccatctccaaacagcattaa

SEQ ID NO: 890 - protein, 623 aa, Homo sapiens
MPPKVTSELLRQLRQAMRNSEYVTEPIQAYIIPSGDAHQSEYIAPCDCRRAFVSGFDGSAGTAIITEEHAAMWTDGRYFLQAAKQMDSNWTLMKMGLKDTPTQEDWLVSVLPEGSRVGVDPLIIPTDYWKKMAKVLRSAGHHLIPVKENLVDKIWTDRPERPCKPLLTLGLDYTGISWKDKVADLRLKMAERNVMWFVVTALDEIAWLFNLRGSDVEHNPVFFSYAIIGLETIMLFIDGDRIDAPSVKEHLLLDLGLEAEYRIQVHPYKSILSELKALCADLSPREKVWVSDKASYAVSETIPKDHRCCMPYTPICIAKAVKNSAESEGMRPAHIKDAVALCELFNWLEKEVPKGGVTEISAADKAEEFRRQQADFVDLSFPTISSTGPNGAIIHYAPVPETNRTLSLDEVYLIDSGAQYKDGTTDVTRTMHFGTPTAYEKECFTYVLKGHIAVSAAVFPTGTKGHLLDSFARSALWDSGLDYLHGTGHGVGSFLNVHEGPCGISYKTFSDEPLEAGMIVTDEPGYYEDGAFGIRIENVVLVVPVKTKYNFNNRGSLTFEPLTLVPIQTKMIDVDSLTDKECDWLNNYHLTCRDVIGKELQKQGRQEALEWLIRETQPISKQH

SEQ ID NO: 891 - DNA, 871 bp, Homo sapiens
  misc_feature (603)..(603): wherein “n” equals either ′C′ or ′T′.
tcctccacctgctggcccctggacacctctgtcaccatgtggttcctggttctgtgcctcgccctgtccctgggggggactggtgctgcgcccccgattcagtcccggattgtgggaggctgggagtgtgagcagcattcccagccctggcaggcggctctgtaccatttcagcactttccagtgtgggggcatcctggtgcaccgccagtgggtgctcacagctgctcattgcatcagcgacaattaccagctctggctgggtcgccacaacttgtttgacgacgaaaacacagcccagtttgttcatgtcagtgagagcttcccacaccctggcttcaacatgagcctcctggagaaccacacccgccaagcagacgaggactacagccacgacctcatgctgctccgcctgacagagcctgctgataccatcacagatgctgtgaaggtcgtggagttgcccaccgaggaacccgaagtggggagcacctgtttggcttccggctggggcagcatcgaaccagagaatttctcatttccagatgatctccagtgtgtggacctcaaaatcctgcctaatgatgagtgcaaaaaagcccangtccagaaggtgacagacttcatgctgtgtgtcggacacctggaaggtggcaaagacacctgtgtgggtgattcagggggcccgctgatgtgtgatggtgtgctccaaggtgtcacatcatggggctacgtcccttgtggcacccccaataagccttctgtcgccgtcagagtgctgtcttatgtgaagtggatcgaggacaccatagcggagaactcctgaacgcccagccctgtcccctacccccagtaaaatcaaatgtgcatcc

SEQ ID NO: 892 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 893 - DNA, 3431 bp, Homo sapiens
  misc_feature (2172)..(2172): wherein “n” equals either ′G′ or ′A′.
caccctatcctacactactaggaacttgcacagtccgcctcgggcagcccaaagctcctctgcccaccctggctcccaaaaccctccaaaacaaaagaccagaaaagcactctccacccagcagccaaacgcctccttcttgacgccagcccccaccctctgtctgctcgagcccaggaaaggcctgaaggaacaggccggggaaggagccctccctctctcccttgtccctccatccacccagcgccggcatctggagaccctatggcccgggctcactggggctgctgcccctggctggtcctcctctgtgcttgtgcctggggccacacaaagccactggaccttggagggcaggatgtgagaaattgttccaccaaccccccttaccttccagttactgtggtcaataccacaatgtcactcacagccctccgccagcagatgcagacccagaatctctcagcctacatcatcccaggcacagatgctcacatgaacgagtacatcggccaacatgacgagaggcgtgcgtggattacaggctttacagggtctgcaggaactgcagtggtgactatgaagaaagcagctgtctggaccgacagtcgctactggactcaggctgagcggcaaatggactgtaattgggagctccataaggaagttggcaccactcctattgtcacctggctcctcaccgagattcccgctggagggcgtgtgggttttgaccccttcctcttgtccattgacacctgggagagttatgatctggccctccaaggctctaacagacagctggtgtccatcacaaccaatcttgtggacctggtatggggatcagagaggccaccggttccaaatcaacccatttatgccctgcaggaggcattcacagggagcacttggcaggagaaagtatctggcgtccgaagccagatgcagaagcatcaaaaggtcccgactgccgtccttctgtcggcgcttgaggagacggcctggctcttcaaccttcgagccagtgacatcccctataaccccttcttctattcctacacgctgctcacagactcttctattaggttgtttgcaaacaagagtcgctttagctccgaaaccttgagctatctgaactccagttgcacaggccccatgtgtgtgcaaatcgaggattacagccaagttcgtgacagcatccaggcctactcattgggagatgtgaggatctggattgggaccagctataccatgtatgggatctatgaaatgataccaagggagaaactcgtgacagacacctactccccagtgatgatgaccaaggcagtgaagaacagcaaggagcaggccctcctcaaggccagccacgtgcgggacgctgtggctgtgatccggtacttggtctggctggagaagaacgtgcccaaaggcacagtggatgagttttcgggggcagagatcgtggacaagttccgaggagaagaacagttctcctccggacccagttttgaaaccatctctgctagtggtttgaatgctgccctggcccactacagcccgaccaaggagctgaaccgcaagctgtcctcagatgagatgtacctgctggactctggggggcagtactgggacgggaccacagacatcaccagaacagtccactggggcaccccctctgcctttcagaaggaggcatatacccgtgtgctgataggaaatattgacctgtccaggctcatctttcccgctgctacatcagggcgaatggtggaggcctttgcccgcagagccttgtgggatgctggtctcaattatggtcatgggacaggccacggcattggcaacttcctgtgtgtgcatgagtggccagtgggattccagtccaacaacatcgctatggccaagggcatgttcacttccattgaacctggttactataaggatggagaatttgggatccgtctcgaagatgtggctctcgtggtagaagcaaagaccaagtacccagggagctacctgacctttgaagtggtatcatttgtgccctatgaccggaacctcatcgatgtcagcctgctgtctcccgagcatctccagtacctgaatcgctactaccagaccatccgggagaaggtgggtccagagctgcagaggcgccagctactagangagttcgagtggcttcaacagcacacagagcccctggccgccagggccccagacaccgcctcctgggcctctgtgttagtggtctccacccttgccatccttggctggagtgtctagaggctccagactctcctgttaaccctccatctagatggggggctcccttgcttagctcccctcaccctgcactgaacataccccaagagcccctgctggcccattgcctagaaacctttgcattcatcctccttctccaagacctatggagaaggtcccaggccccaggaaacacagggcttcttggccccagatggcacctccctgcaccccggggttgtataccacaccctgggcccctaatcccaggccccgaaataggaaagccagctagtctcttctcttctgtgatctcagtaggcctaacctataacctaacacagactgctacagctgctcccctcccgccaaacaaagccccaagaaaacaatgcccctaccacccaagggtgccatggtcccgggaaaacccaacctgtcaccgcgtgttgggcgtaaccagaactgttcccccccaccagggcttaaaaatcgcccccactttttaaccatcgtccattaaccacctggtgggcatagccagagctgttcgaacccagccagggatgaaaaatcaacccccgacatggaacccatgattcctaaacccggggtaggttccatgccaagtaacagcagagggagttaagccataggaatttggctgtggagtaagagggaatgcggtgaggcagtgtggaatatgaccctaccagaggttggagaacaaacttgggcagccggaacccgtcactattttagattcctggcattcgaggagccctttgaactttccaaagtgcagccacagctacaatgctgttaaatcctcccacatttcttggatgccccttcaccttgtgtggacagtgtctggtttccccattttacagacaggaaaactgagcttcagacagggggtgggctttgcctaaggacacacaaatttggttgggagttgatggggccagatgagccagcattccagctgtttcacccttcagcaacatgcagagtccctgagcccacctcccagccctctcctcattctctgaacccactgtggtgagaagaatttgctccggccaaattggccgttagccacctgggtccacatcctgctaagacgtttaaaacagcctaacaaagacacttgcctgtgg

SEQ ID NO: 894 - protein, 674 aa, Homo sapiens
MARAHWGCCPWLVLLCACAWGHTKPLDLGGQDVRNCSTNPPYLPVTVVNTTMSLTALRQQMQTQNLSAYIIPGTDAHMNEYIGQHDERRAWITGFTGSAGTAVVTMKKAAVWTDSRYWTQAERQMDCNWELHKEVGTTPIVTWLLTEIPAGGRVGFDPFLLSIDTWESYDLALQGSNRQLVSITTNLVDLVWGSERPPVPNQPIYALQEAFTGSTWQEKVSGVRSQMQKHQKVPTAVLLSALEETAWLFNLRASDIPYNPFFYSYTLLTDSSIRLFANKSRFSSETLSYLNSSCTGPMCVQIEDYSQVRDSIQAYSLGDVRIWIGTSYTMYGIYEMIPREKLVTDTYSPVMMTKAVKNSKEQALLKASHVRDAVAVIRYLVWLEKNVPKGTVDEFSGAEIVDKFRGEEQFSSGPSFETISASGLNAALAHYSPTKELNRKLSSDEMYLLDSGGQYWDGTTDITRTVHWGTPSAFQKEAYTRVLIGNIDLSRLIFPAATSGRMVEAFARRALWDAGLNYGHGTGHGIGNFLCVHEWPVGFQSNNIAMAKGMFTSIEPGYYKDGEFGIRLEDVALVVEAKTKYPGSYLTFEVVSFVPYDRNLIDVSLLSPEHLQYLNRYYQTIREKVGPELQRRQLLEEFEWLQQHTEPLAARAPDTASWASVLVVSTLAILGWSV

SEQ ID NO: 895 - DNA, 2954 bp, Homo sapiens
  misc_feature (822)..(822): wherein “n” equals either ′C′ or ′T′.
tgggccgcagccctccccgccccgccgaccgcggtcacacactctcggagcctccccgtgagcgggagcgcggcgcacggcgatgcgccgaggcgggcgctgaggcggcgccgcgcgagcagcagcagaggcggcggcggcccccagcccagcccggcgccgccgccgagcccgggccccaaggtgcggcggcgccccaagttcccgccatgagcagccggctcggggggctccgcggccccggggactcccgccccgccgggcgcgaccgcagcgccccgcggccccgacgcgcttaacgttgtcgcttgccggtcccgccaccgccgcctccgccgccgctcgcgtcctcgccgccaccgcctcggccgctgcagctccgccggcagcatgtctcgaaggaagatttcgtccgagtccttcagctccctgggctccgactacctggagaccagcccggaggaggagggggagtgccccctgtctaggctctgctggaatggcagccggagcccgcccgggccgctggagcccagcccggccgcagctgccgctgccgccgccccggccccgaccccggctgcttctgccgccgccgccgctgccactgccggggccaggagggtgcagcgccggaggcgggtcaacctggactcgctgggcgagagcatcagccgcctgacggcgccctcgcctcagacgatacagcagactctcaagaggacactgcagtattatgaacatcaagttattggttacagggatgcagaaaagaatttccacaatatctctaacagatgctcctatgcagaccactccaanaangaagaaattgaagatgtctcaggaattcttcagtgtactgctaatatactcggtttgaagtttgaggaaattcaaaaaagatttggtgaagagttctttaatatatgctttcatgagaatgagagagtccttcgagctgtaggtggcactttgcaggacttttttaacggctttgatgctttgttggaacacattagaacttcttttggaaaacaggccactctggagtcaccatctttcctatgcaaagagctccctgaaggtactctcatgctccactacttccaccctcaccatattgtggggtttgcaatgctggggatgattaaggctgcaggaaagaagatctatcggctggatgtggaagtggaacaggttgcaaatgagaagctatgctctgatgtttcaaacccaggcaattgtagctgtcttactttccttatcaaagaatgtgaaaatactaatatcatgaagaaccttccacagggaacctcccaagttcctgcggacctcagaattagcatcaacaccttctgtagagccttccctttccacttgatgtttgatcccagcatgtcagtccttcagttgggggaaggtctaaggaagcagcttcgatgtgacactcacaaagtgctcaagtttgaggactgcttcgagattgtatctccaaaggttaatgccacctttgaaagggtcctgctgcgactgtctaccccgtttgtgattagaaccaagcctgaggcttctggctctgaaaataaagacaaggtgatggaagtcaaaggacaaatgatccatgttccagaatcaaattccattttatttttgggctctccatgtgtggacaagttggatgaactcatgggccgagggctacatctctcagacatccctatccatgatgccacccgagatgtcattttggttggtgagcaggcaaaggcccaagatgggttgaagaaaaggatggataaattaaaggcaactttagaaagaactcaccaggccctggaagaagagaaaaagaagacagtggatcttctatattctattttccctggtgatgtagcccagcaattatggcaagggcagcaagtacaggccagaaagtttgatgatgtcaccatgctcttttcagacattgttggcttcacagccatatgtgcccagtgtactcccatgcaagtaatcagcatgctgaatgaactgtacaccagatttgaccaccagtgtggatttttggatatttataaggtggaaacaataggtgatgcctactgtgttgcagcagggctccacagaaaaagcctctgccatgctaaacccattgctctgatggcnttgaagatgatggaactttcagaagaggtgctgacacctgatggaagaccgattcagatgaggataggaattcactcaggctccgtgctggctggagttgttggggtgcgaatgccacgttattgcctgtttggaaataatgtcacactggcaagcaaattcgagtcgggaagtcaccctcggcgcatcaatgtcagcccaaccacttaccaattattaaaacgagaagaaagtttcacattcattccgcggtctcgtgaagagcttccagacaactttccaaaggaaattcctgggatctgctatttcctggaggtaaggactggtccaaagccaccaaagccttctctttcttcgtcgagaataaaaaaggtttcctacaacatcggcaccatgttcctccgggagacaagcctctgagacctgctacagatcaaagactcctccaaaaagcacaagcccagaacatgggtcaccaatggggggtggaaagagattgtgtctctttcattgctttgttgagaacaagcagcaaaatttctgtattatgtcaggcaataatcctactaaaaggtggaggtgaccgctgtcaataaaaagccggaggatgagggaaataagatgtgtccattcatatgagtggttttggtcatatatatacacatatattttaattacaagtgtgggtcccctttcagaactaaccaataaatagattccatgttttcttgtttatcacacatacaagtatctttccctatatatttgtaccacttttgagagcc

SEQ ID NO: 896 - protein, 732 aa, Homo sapiens
MSRRKISSESFSSLGSDYLETSPEEEGECPLSRLCWNGSRSPPGPLEPSPAAAAAAAAPAPTPAASAAAAAATAGARRVQRRRRVNLDSLGESISRLTAPSPQTIQQTLKRTLQYYEHQVIGYRDAEKNFHNISNRCSYADHSNKEEIEDVSGILQCTANILGLKFEEIQKRFGEEFFNICFHENERVLRAVGGTLQDFFNGFDALLEHIRTSFGKQATLESPSFLCKELPEGTLMLHYFHPHHIVGFAMLGMIKAAGKKIYRLDVEVEQVANEKLCSDVSNPGNCSCLTFLIKECENTNIMKNLPQGTSQVPADLRISINTFCRAFPFHLMFDPSMSVLQLGEGLRKQLRCDTHKVLKFEDCFEIVSPKVNATFERVLLRLSTPFVIRTKPEASGSENKDKVMEVKGQMIHVPESNSILFLGSPCVDKLDELMGRGLHLSDIPIHDATRDVILVGEQAKAQDGLKKRMDKLKATLERTHQALEEEKKKTVDLLYSIFPGDVAQQLWQGQQVQARKFDDVTMLFSDIVGFTAICAQCTPMQVISMLNELYTRFDHQCGFLDIYKVETIGDAYCVAAGLHRKSLCHAKPIALMALKMMELSEEVLTPDGRPIQMRIGIHSGSVLAGVVGVRMPRYCLFGNNVTLASKFESGSHPRRINVSPTTYQLLKREESFTFIPRSREELPDNFPKEIPGICYFLEVRTGPKPPKPSLSSSRIKKVSYNIGTMFLRETSL

SEQ ID NO: 897 - DNA, 39 bp, Homo sapiens
caggaaacagctatgacctcagtcccttgtctgcaattc

SEQ ID NO: 898 - DNA, 39 bp, Homo sapiens
caggaaacagctatgaccagcattaagcaatcggtgaaa

SEQ ID NO: 899 - DNA, 39 bp, Homo sapiens
caggaaacagctatgaccagaaagccaggaccaagagac

SEQ ID NO: 900 - DNA, 39 bp, Homo sapiens
caggaaacagctatgacccaggcatatcccagtgaggta

SEQ ID NO: 901 - DNA, 39 bp, Homo sapiens
caggaaacagctatgaccactgaagaagggaggctctgt

SEQ ID NO: 902 - DNA, 39 bp, Homo sapiens
caggaaacagctatgaccagggctcaactctggaatcat

SEQ ID NO: 903 - DNA, 39 bp, Homo sapiens
caggaaacagctatgacccggatctttaagcaataggcc

SEQ ID NO: 904 - DNA, 39 bp, Homo sapiens
caggaaacagctatgaccataggtgtgagccactgcact

SEQ ID NO: 905 - DNA, 39 bp, Homo sapiens
caggaaacagctatgaccgacctgagactgcagacagct

SEQ ID NO: 906 - DNA, 39 bp, Homo sapiens
caggaaacagctatgacctgaggacaactggacgatttt

SEQ ID NO: 907 - DNA, 39 bp, Homo sapiens
caggaaacagctatgaccctttccaagaaaggggctatg

SEQ ID NO: 908 - DNA, 39 bp, Homo sapiens
caggaaacagctatgaccagtgtggcctgtgttctctgt

SEQ ID NO: 909 - DNA, 39 bp, Homo sapiens
caggaaacagctatgaccccaacaatgtatgttgggtcc

SEQ ID NO: 910 - DNA, 39 bp, Homo sapiens
caggaaacagctatgaccctccagctgccatcagaatag

SEQ ID NO: 911 - DNA, 39 bp, Homo sapiens
caggaaacagctatgacccagctgctgtattgacccact

SEQ ID NO: 912 - DNA, 39 bp, Homo sapiens
caggaaacagctatgaccttcattgtccatcgagagctt

SEQ ID NO: 913 - DNA, 39 bp, Homo sapiens; identical to SEQ ID NO: 912

SEQ ID NO: 914 - DNA, 39 bp, Homo sapiens
caggaaacagctatgaccggtaacgggtacatggaggtt

SEQ ID NO: 915 - DNA, 39 bp, Homo sapiens; identical to SEQ ID NO: 914

SEQ ID NO: 916 - DNA, 39 bp, Homo sapiens; identical to SEQ ID NO: 914

SEQ ID NO: 917 - DNA, 39 bp, Homo sapiens; identical to SEQ ID NO: 914

SEQ ID NO: 918 - DNA, 39 bp, Homo sapiens
caggaaacagctatgaccttcttacctgctgcagctcat

SEQ ID NO: 919 - DNA, 39 bp, Homo sapiens
caggaaacagctatgacccacagcttgaacacaggtctg

SEQ ID NO: 920 - DNA, 39 bp, Homo sapiens
caggaaacagctatgacctggctgatttttgtccacttc

SEQ ID NO: 921 - DNA, 39 bp, Homo sapiens
caggaaacagctatgaccctgacacccattccacagact

SEQ ID NO: 922 - DNA, 39 bp, Homo sapiens
caggaaacagctatgaccacaaagtcccagaccagacct

SEQ ID NO: 923 - DNA, 39 bp, Homo sapiens
caggaaacagctatgacctcaggtaaaggctaagaggcc

SEQ ID NO: 924 - DNA, 39 bp, Homo sapiens
caggaaacagctatgaccaatactgcctccacctcaaca

SEQ ID NO: 925 - DNA, 39 bp, Homo sapiens
caggaaacagctatgaccgactcacctgttcaggatcca

SEQ ID NO: 926 - DNA, 39 bp, Homo sapiens; identical to SEQ ID NO: 925

SEQ ID NO: 927 - DNA, 39 bp, Homo sapiens
caggaaacagctatgaccacttccaaacccatgagagct

SEQ ID NO: 928 - DNA, 39 bp, Homo sapiens
caggaaacagctatgaccaaagcttggtgctgtggagta

SEQ ID NO: 929 - DNA, 39 bp, Homo sapiens; identical to SEQ ID NO: 928

SEQ ID NO: 930 - DNA, 39 bp, Homo sapiens
caggaaacagctatgacccatctgtgaaatgggtgtgtg

SEQ ID NO: 931 - DNA, 39 bp, Homo sapiens
caggaaacagctatgacctggcctaggattctctccttt

SEQ ID NO: 932 - DNA, 39 bp, Homo sapiens
caggaaacagctatgacccagcaaagtggagattggaac

SEQ ID NO: 933 - DNA, 39 bp, Homo sapiens; identical to SEQ ID NO: 932

SEQ ID NO: 934 - DNA, 39 bp, Homo sapiens
caggaaacagctatgacccttgaatacgtcctcggacag

SEQ ID NO: 935 - DNA, 39 bp, Homo sapiens; identical to SEQ ID NO: 934

SEQ ID NO: 936 - DNA, 39 bp, Homo sapiens
caggaaacagctatgaccctgtaggagatggcgtcaaac

SEQ ID NO: 937 - DNA, 39 bp, Homo sapiens
caggaaacagctatgaccgatttcaacaccatcgtgacc

SEQ ID NO: 938 - DNA, 39 bp, Homo sapiens
caggaaacagctatgacctcatgagcaatcacagtgacc

SEQ ID NO: 939 - DNA, 39 bp, Homo sapiens
caggaaacagctatgaccgtaggtcaccagtccccagtt

SEQ ID NO: 940 - DNA, 39 bp, Homo sapiens; identical to SEQ ID NO: 939

SEQ ID NO: 941 - DNA, 39 bp, Homo sapiens; identical to SEQ ID NO: 939

SEQ ID NO: 942 - DNA, 39 bp, Homo sapiens
caggaaacagctatgaccgcaaagaagttaaggatgggg

SEQ ID NO: 943 - DNA, 39 bp, Homo sapiens
caggaaacagctatgaccacattccagttggggtcttct

SEQ ID NO: 944 - DNA, 39 bp, Homo sapiens
caggaaacagctatgaccactcacctttgggaagcatgt

SEQ ID NO: 945 - DNA, 39 bp, Homo sapiens
caggaaacagctatgaccgacctcgggtgagtttctctc

SEQ ID NO: 946 - DNA, 39 bp, Homo sapiens
caggaaacagctatgacctccatctcatactggctgtcc

SEQ ID NO: 947 - DNA, 39 bp, Homo sapiens
caggaaacagctatgaccgcccagggacttggaaatata

SEQ ID NO: 948 - DNA, 39 bp, Homo sapiens
caggaaacagctatgacccccggctcctattaagaacac

SEQ ID NO: 949 - DNA, 39 bp, Homo sapiens; identical to SEQ ID NO: 948

SEQ ID NO: 950 - DNA, 39 bp, Homo sapiens
caggaaacagctatgacccttcaagagcagggagttcct

SEQ ID NO: 951 - DNA, 39 bp, Homo sapiens
caggaaacagctatgaccctttccagctgtgaccttgag

SEQ ID NO: 952 - DNA, 39 bp, Homo sapiens
caggaaacagctatgacctgcctctcaggtttgttgaac

SEQ ID NO: 953 - DNA, 39 bp, Homo sapiens; identical to SEQ ID NO: 952

SEQ ID NO: 954 - DNA, 44 bp, Homo sapiens
caggaaacagctatgacctgttgaacagatttagtgagaaaaca

SEQ ID NO: 955 - DNA, 39 bp, Homo sapiens
caggaaacagctatgacctaggagttcaagaccagcctg

SEQ ID NO: 956 - DNA, 39 bp, Homo sapiens; identical to SEQ ID NO: 955

SEQ ID NO: 957 - DNA, 39 bp, Homo sapiens
caggaaacagctatgacccatggtggctcacatgtgtaa

SEQ ID NO: 958 - DNA, 39 bp, Homo sapiens
caggaaacagctatgaccctcagttcccatggcttcata

SEQ ID NO: 959 - DNA, 39 bp, Homo sapiens; identical to SEQ ID NO: 958

SEQ ID NO: 960 - DNA, 39 bp, Homo sapiens; identical to SEQ ID NO: 958

SEQ ID NO: 961 - DNA, 39 bp, Homo sapiens; identical to SEQ ID NO: 506

SEQ ID NO: 962 - DNA, 39 bp, Homo sapiens
caggaaacagctatgaccatgtgtcaccatgattctgca

SEQ ID NO: 963 - DNA, 39 bp, Homo sapiens
caggaaacagctatgaccaacgttggttgaactcattgc

SEQ ID NO: 964 - DNA, 39 bp, Homo sapiens
caggaaacagctatgaccgcaactagcaagaaagtcccc

SEQ ID NO: 965 - DNA, 39 bp, Homo sapiens
caggaaacagctatgaccgacatagtcatcccggtcaga

SEQ ID NO: 966 - DNA, 39 bp, Homo sapiens
caggaaacagctatgaccgcaaaactaatttggccacag

SEQ ID NO: 967 - DNA, 39 bp, Homo sapiens; identical to SEQ ID NO: 966

SEQ ID NO: 968 - DNA, 39 bp, Homo sapiens
caggaaacagctatgacctcactcctgttaccttggcac

SEQ ID NO: 969 - DNA, 39 bp, Homo sapiens
caggaaacagctatgaccagataagaggccttgcaggag

SEQ ID NO: 970 - DNA, 39 bp, Homo sapiens
caggaaacagctatgaccaagggttaaattgccatccac

SEQ ID NO: 971 - DNA, 39 bp, Homo sapiens; identical to SEQ ID NO: 970

SEQ ID NO: 972 - DNA, 39 bp, Homo sapiens; identical to SEQ ID NO: 517

SEQ ID NO: 973 - DNA, 39 bp, Homo sapiens
caggaaacagctatgaccccaaaagtgtcattgtggctt

SEQ ID NO: 974 - DNA, 39 bp, Homo sapiens
caggaaacagctatgaccaatcagtggccacaacaaaaa

SEQ ID NO: 975 - DNA, 39 bp, Homo sapiens; identical to SEQ ID NO: 974

SEQ ID NO: 976 - DNA, 39 bp, Homo sapiens
caggaaacagctatgaccttccgctatagcttcatgtgg

SEQ ID NO: 977 - DNA, 39 bp, Homo sapiens; identical to SEQ ID NO: 976

SEQ ID NO: 978 - DNA, 39 bp, Homo sapiens
caggaaacagctatgaccccttacctgcactcagctttg

SEQ ID NO: 979 - DNA, 39 bp, Homo sapiens
caggaaacagctatgacctcaaatttggtcgatttgagc

SEQ ID NO: 980 - DNA, 39 bp, Homo sapiens
caggaaacagctatgaccgttgtcccagcataggaaaca

SEQ ID NO: 981 - DNA, 39 bp, Homo sapiens
caggaaacagctatgaccgcaaaatcagttgtgcctttc

SEQ ID NO: 982 - DNA, 39 bp, Homo sapiens
caggaaacagctatgacctaggcgaaatccatgatgaag

SEQ ID NO: 983 - DNA, 39 bp, Homo sapiens
caggaaacagctatgacctcccattgggtgtttctagtg

SEQ ID NO: 984 - DNA, 39 bp, Homo sapiens
caggaaacagctatgacccagaagccatgaagtctggtc

SEQ ID NO: 985 - DNA, 39 bp, Homo sapiens
caggaaacagctatgacccaggagttctcacagagtccg

SEQ ID NO: 986 - DNA, 39 bp, Homo sapiens
caggaaacagctatgaccgcggcatcagttacaaaacat

SEQ ID NO: 987 - DNA, 39 bp, Homo sapiens
caggaaacagctatgacccttgtgctgtgctgaaatctg

SEQ ID NO: 988 - DNA, 39 bp, Homo sapiens
caggaaacagctatgaccgggtcatctctctgacagtgc

SEQ ID NO: 989 - DNA, 39 bp, Homo sapiens; identical to SEQ ID NO: 534

SEQ ID NO: 990 - DNA, 39 bp, Homo sapiens; identical to SEQ ID NO: 534

SEQ ID NO: 991 - DNA, 39 bp, Homo sapiens
caggaaacagctatgaccgtaaaccaggactgctgtgga

SEQ ID NO: 992 - DNA, 39 bp, Homo sapiens
caggaaacagctatgacccttgtccctggatgaggtgta

SEQ ID NO: 993 - DNA, 39 bp, Homo sapiens; identical to SEQ ID NO: 538

SEQ ID NO: 994 - DNA, 39 bp, Homo sapiens
caggaaacagctatgaccggagtttcgcaggtaaggatc

SEQ ID NO: 995 - DNA, 39 bp, Homo sapiens; identical to SEQ ID NO: 539

SEQ ID NO: 996 - DNA, 39 bp, Homo sapiens
caggaaacagctatgacctaggaggggatgcttttgaat

SEQ ID NO: 997 - DNA, 39 bp, Homo sapiens
caggaaacagctatgaccttggccattaatttcttgctc

SEQ ID NO: 998 - DNA, 39 bp, Homo sapiens
caggaaacagctatgaccgtatgccttacacccccatct

SEQ ID NO: 999 - DNA, 39 bp, Homo sapiens
caggaaacagctatgacctagctatatgggtcgccagtg

SEQ ID NO: 1000 - DNA, 39 bp, Homo sapiens
caggaaacagctatgaccagatttctggcttggcagatt

SEQ ID NO: 1001 - DNA, 39 bp, Homo sapiens
caggaaacagctatgaccatcagatgtggagcacaatcc

SEQ ID NO: 1002 - DNA, 39 bp, Homo sapiens
caggaaacagctatgacctcaagtggcttggacacttct

SEQ ID NO: 1003 - DNA, 39 bp, Homo sapiens
caggaaacagctatgaccggaagaaaatggccaaagttc

SEQ ID NO: 1004 - DNA, 39 bp, Homo sapiens; identical to SEQ ID NO: 1003

SEQ ID NO: 1005 - DNA, 39 bp, Homo sapiens
caggaaacagctatgaccgtccctttctgcttgggtaag

SEQ ID NO: 1006 - DNA, 39 bp, Homo sapiens; identical to SEQ ID NO: 1005

SEQ ID NO: 1007 - DNA, 39 bp, Homo sapiens
caggaaacagctatgacccccagaggtttctttggagtc

SEQ ID NO: 1008 - DNA, 39 bp, Homo sapiens
caggaaacagctatgaccaaagggaacagctctctctgc

SEQ ID NO: 1009 - DNA, 39 bp, Homo sapiens
caggaaacagctatgacctagctgaacttttctggccac

SEQ ID NO: 1010 - DNA, 40 bp, Homo sapiens
caggaaacagctatgacctcacccacaaatgttgtctagg

SEQ ID NO: 1011 - DNA, 40 bp, Homo sapiens; identical to SEQ ID NO: 1010

SEQ ID NO: 1012 - DNA, 39 bp, Homo sapiens; identical to SEQ ID NO: 556

SEQ ID NO: 1013 - DNA, 39 bp, Homo sapiens
caggaaacagctatgacctttgactcctagtggacggaa

SEQ ID NO: 1014 - DNA, 39 bp, Homo sapiens
caggaaacagctatgaccccagtgtgtggacctcaaaat

SEQ ID NO: 1015 - DNA, 39 bp, Homo sapiens; identical to SEQ ID NO: 1014

SEQ ID NO: 1016 - DNA, 39 bp, Homo sapiens; identical to SEQ ID NO: 559

SEQ ID NO: 1017 - DNA, 39 bp, Homo sapiens
caggaaacagctatgacccctgacagagcctgctgatac

SEQ ID NO: 1018 - DNA, 39 bp, Homo sapiens
caggaaacagctatgaccaaatgtgatgttcaaggtcgc

SEQ ID NO: 1019 - DNA, 39 bp, Homo sapiens; identical to SEQ ID NO: 1018

SEQ ID NO: 1020 - DNA, 39 bp, Homo sapiens
caggaaacagctatgacctttcttagccaggtccctgat

SEQ ID NO: 1021 - DNA, 39 bp, Homo sapiens
caggaaacagctatgaccaccctgggcaacagaataaga

SEQ ID NO: 1022 - DNA, 39 bp, Homo sapiens
caggaaacagctatgaccgaaggaggaaaaaggaggagg

SEQ ID NO: 1023 - DNA, 39 bp, Homo sapiens
caggaaacagctatgacccctcacaccctatcctacacg

SEQ ID NO: 1024 - DNA, 39 bp, Homo sapiens
caggaaacagctatgaccgagccctccaaaacaaaagac

SEQ ID NO: 1025 - DNA, 39 bp, Homo sapiens
caggaaacagctatgaccctcctttgcagaacagtccag

SEQ ID NO: 1026 - DNA, 39 bp, Homo sapiens
caggaaacagctatgacctgattcaggacacctttctgc

SEQ ID NO: 1027 - DNA, 39 bp, Homo sapiens
caggaaacagctatgacctattgtcacctggctcctcac

SEQ ID NO: 1028 - DNA, 39 bp, Homo sapiens
caggaaacagctatgacctgttagagtaggctcagggca

SEQ ID NO: 1029 - DNA, 39 bp, Homo sapiens
caggaaacagctatgacctttgcaaacaagagtcgcttt

SEQ ID NO: 1030 - DNA, 39 bp, Homo sapiens
caggaaacagctatgaccggactatggtgacagctggag

SEQ ID NO: 1031 - DNA, 39 bp, Homo sapiens
caggaaacagctatgaccgaggctccagactctcctgtt

SEQ ID NO: 1032 - DNA, 39 bp, Homo sapiens
caggaaacagctatgaccccttcaccttgtgtggacagt

SEQ ID NO: 1033 - DNA, 39 bp, Homo sapiens
caggaaacagctatgaccgatgttttgccgacatgtttt

SEQ ID NO: 1034 - DNA, 39 bp, Homo sapiens
caggaaacagctatgaccacgttattgcctgtttggaaa

SEQ ID NO: 1035 - DNA, 39 bp, Homo sapiens; identical to SEQ ID NO: 580

SEQ ID NO: 1036 - DNA, 39 bp, Homo sapiens; identical to SEQ ID NO: 580

SEQ ID NO: 1037 - DNA, 39 bp, Homo sapiens
caggaaacagctatgacccaaatgctgatgtgaatggtg

SEQ ID NO: 1038 - DNA, 39 bp, Homo sapiens
caggaaacagctatgaccgccttgatgaagtgctctcag

SEQ ID NO: 1039 - DNA, 39 bp, Homo sapiens
caggaaacagctatgaccaccatactcagctttggggtt

SEQ ID NO: 1040 - DNA, 39 bp, Homo sapiens
caggaaacagctatgaccggtgaccgctgtcaataaaaa

SEQ ID NO: 1041 - DNA, 39 bp, Homo sapiens
caggaaacagctatgacccaagacagcactgttgctgag

SEQ ID NO: 1042 - DNA, 39 bp, Homo sapiens; identical to SEQ ID NO: 1041

SEQ ID NO: 1043 - DNA, 38 bp, Homo sapiens
caggaaacagctatgaccaaggacaggggcaacatttt

SEQ ID NO: 1044 - DNA, 39 bp, Homo sapiens; identical to SEQ ID NO: 897

SEQ ID NO: 1045 - DNA, 39 bp, Homo sapiens; identical to SEQ ID NO: 898

SEQ ID NO: 1046 - DNA, 39 bp, Homo sapiens; identical to SEQ ID NO: 899

SEQ ID NO: 1047 - DNA, 39 bp, Homo sapiens; identical to SEQ ID NO: 900

SEQ ID NO: 1048 - DNA, 39 bp, Homo sapiens; identical to SEQ ID NO: 901

SEQ ID NO: 1049 - DNA, 39 bp, Homo sapiens; identical to SEQ ID NO: 902

SEQ ID NO: 1050 - DNA, 39 bp, Homo sapiens; identical to SEQ ID NO: 903

SEQ ID NO: 1051 - DNA, 39 bp, Homo sapiens; identical to SEQ ID NO: 904

SEQ ID NO: 1052 - DNA, 39 bp, Homo sapiens; identical to SEQ ID NO: 905

SEQ ID NO: 1053 - DNA, 39 bp, Homo sapiens; identical to SEQ ID NO: 906

SEQ ID NO: 1054 - DNA, 39 bp, Homo sapiens; identical to SEQ ID NO: 907

SEQ ID NO: 1055 - DNA, 39 bp, Homo sapiens; identical to SEQ ID NO: 908

SEQ ID NO: 1056 - DNA, 39 bp, Homo sapiens; identical to SEQ ID NO: 909

SEQ ID NO: 1057 - DNA, 39 bp, Homo sapiens; identical to SEQ ID NO: 910

SEQ ID NO: 1058 - DNA, 39 bp, Homo sapiens; identical to SEQ ID NO: 911

SEQ ID NO: 1059 - DNA, 39 bp, Homo sapiens; identical to SEQ ID NO: 912

SEQ ID NO: 1060 - DNA, 39 bp, Homo sapiens; identical to SEQ ID NO: 912

SEQ ID NO: 1061 - DNA, 39 bp, Homo sapiens; identical to SEQ ID NO: 914

SEQ ID NO: 1062 - DNA, 39 bp, Homo sapiens; identical to SEQ ID NO: 914

SEQ ID NO: 1063 - DNA, 39 bp, Homo sapiens; identical to SEQ ID NO: 914

SEQ ID NO: 1064 - DNA, 39 bp, Homo sapiens; identical to SEQ ID NO: 914

SEQ ID NO: 1065 - DNA, 39 bp, Homo sapiens; identical to SEQ ID NO: 918

SEQ ID NO: 1066 - DNA, 39 bp, Homo sapiens; identical to SEQ ID NO: 919

SEQ ID NO: 1067 - DNA, 39 bp, Homo sapiens; identical to SEQ ID NO: 920

SEQ ID NO: 1068 - DNA, 39 bp, Homo sapiens; identical to SEQ ID NO: 921

SEQ ID NO: 1069 - DNA, 39 bp, Homo sapiens; identical to SEQ ID NO: 922

SEQ ID NO: 1070 - DNA, 39 bp, Homo sapiens; identical to SEQ ID NO: 923

SEQ ID NO: 1071 - DNA, 39 bp, Homo sapiens; identical to SEQ ID NO: 924

SEQ ID NO: 1072 - DNA, 39 bp, Homo sapiens; identical to SEQ ID NO: 925

SEQ ID NO: 1073 - DNA, 39 bp, Homo sapiens; identical to SEQ ID NO: 925

SEQ ID NO: 1074 - DNA, 39 bp, Homo sapiens; identical to SEQ ID NO: 927

SEQ ID NO: 1075 - DNA, 39 bp, Homo sapiens; identical to SEQ ID NO: 928

SEQ ID NO: 1076 - DNA, 39 bp, Homo sapiens; identical to SEQ ID NO: 928

SEQ ID NO: 1077 - DNA, 39 bp, Homo sapiens; identical to SEQ ID NO: 930

SEQ ID NO: 1078 - DNA, 39 bp, Homo sapiens; identical to SEQ ID NO: 931

SEQ ID NO: 1079 - DNA, 39 bp, Homo sapiens; identical to SEQ ID NO: 932

SEQ ID NO: 1080 - DNA, 39 bp, Homo sapiens; identical to SEQ ID NO: 932

SEQ ID NO: 1081 - DNA, 39 bp, Homo sapiens; identical to SEQ ID NO: 934

SEQ ID NO: 1082 - DNA, 39 bp, Homo sapiens; identical to SEQ ID NO: 934

SEQ ID NO: 1083 - DNA, 39 bp, Homo sapiens; identical to SEQ ID NO: 936

SEQ ID NO: 1084 - DNA, 39 bp, Homo sapiens; identical to SEQ ID NO: 937

SEQ ID NO: 1085 - DNA, 39 bp, Homo sapiens; identical to SEQ ID NO: 938

SEQ ID NO: 1086 - DNA, 39 bp, Homo sapiens; identical to SEQ ID NO: 939

SEQ ID NO: 1087 - DNA, 39 bp, Homo sapiens; identical to SEQ ID NO: 939

SEQ ID NO: 1088 - DNA, 39 bp, Homo sapiens; identical to SEQ ID NO: 939

SEQ ID NO: 1089 - DNA, 39 bp, Homo sapiens; identical to SEQ ID NO: 942

SEQ ID NO: 1090 - DNA, 39 bp, Homo sapiens; identical to SEQ ID NO: 943

SEQ ID NO: 1091 - DNA, 39 bp, Homo sapiens; identical to SEQ ID NO: 944

SEQ ID NO: 1092 - DNA, 39 bp, Homo sapiens; identical to SEQ ID NO: 945

SEQ ID NO: 1093 - DNA, 39 bp, Homo sapiens; identical to SEQ ID NO: 946

SEQ ID NO: 1094 - DNA, 39 bp, Homo sapiens; identical to SEQ ID NO: 947

SEQ ID NO: 1095 - DNA, 39 bp, Homo sapiens; identical to SEQ ID NO: 948

SEQ ID NO: 1096 - DNA, 39 bp, Homo sapiens; identical to SEQ ID NO: 948

SEQ ID NO: 1097 - DNA, 39 bp, Homo sapiens; identical to SEQ ID NO: 950

SEQ ID NO: 1098 - DNA, 39 bp, Homo sapiens; identical to SEQ ID NO: 951

SEQ ID NO: 1099 - DNA, 39 bp, Homo sapiens; identical to SEQ ID NO: 952

SEQ ID NO: 1100 - DNA, 39 bp, Homo sapiens; identical to SEQ ID NO: 952

SEQ ID NO: 1101 - DNA, 44 bp, Homo sapiens; identical to SEQ ID NO: 954

SEQ ID NO: 1102 - DNA, 39 bp, Homo sapiens; identical to SEQ ID NO: 955

SEQ ID NO: 1103 - DNA, 39 bp, Homo sapiens; identical to SEQ ID NO: 955

SEQ ID NO: 1104 - DNA, 39 bp, Homo sapiens; identical to SEQ ID NO: 957

SEQ ID NO: 1105 - DNA, 39 bp, Homo sapiens; identical to SEQ ID NO: 958

SEQ ID NO: 1106 - DNA, 39 bp, Homo sapiens; identical to SEQ ID NO: 958

SEQ ID NO: 1107 - DNA, 39 bp, Homo sapiens; identical to SEQ ID NO: 958

SEQ ID NO: 1108 - DNA, 39 bp, Homo sapiens; identical to SEQ ID NO: 506

SEQ ID NO: 1109 - DNA, 39 bp, Homo sapiens; identical to SEQ ID NO: 962

SEQ ID NO: 1110 - DNA, 39 bp, Homo sapiens; identical to SEQ ID NO: 963

SEQ ID NO: 1111 - DNA, 39 bp, Homo sapiens; identical to SEQ ID NO: 964

SEQ ID NO: 1112 - DNA, 39 bp, Homo sapiens; identical to SEQ ID NO: 965

SEQ ID NO: 1113 - DNA, 39 bp, Homo sapiens; identical to SEQ ID NO: 966

SEQ ID NO: 1114 - DNA, 39 bp, Homo sapiens; identical to SEQ ID NO: 966

SEQ ID NO: 1115 - DNA, 39 bp, Homo sapiens; identical to SEQ ID NO: 968

SEQ ID NO: 1116 - DNA, 39 bp, Homo sapiens; identical to SEQ ID NO: 969

SEQ ID NO: 1117 - DNA, 39 bp, Homo sapiens; identical to SEQ ID NO: 970

SEQ ID NO: 1118 - DNA, 39 bp, Homo sapiens; identical to SEQ ID NO: 970

SEQ ID NO: 1119 - DNA, 39 bp, Homo sapiens; identical to SEQ ID NO: 517

SEQ ID NO: 1120 - DNA, 39 bp, Homo sapiens; identical to SEQ ID NO: 973

SEQ ID NO: 1121 - DNA, 39 bp, Homo sapiens; identical to SEQ ID NO: 974

SEQ ID NO: 1122 - DNA, 39 bp, Homo sapiens; identical to SEQ ID NO: 974

SEQ ID NO: 1123 - DNA, 39 bp, Homo sapiens; identical to SEQ ID NO: 976

SEQ ID NO: 1124 - DNA, 39 bp, Homo sapiens; identical to SEQ ID NO: 976

SEQ ID NO: 1125 - DNA, 39 bp, Homo sapiens; identical to SEQ ID NO: 978

SEQ ID NO: 1126 - DNA, 39 bp, Homo sapiens; identical to SEQ ID NO: 979

SEQ ID NO: 1127 - DNA, 39 bp, Homo sapiens; identical to SEQ ID NO: 980

SEQ ID NO: 1128 - DNA, 39 bp, Homo sapiens; identical to SEQ ID NO: 981

SEQ ID NO: 1129 - DNA, 39 bp, Homo sapiens; identical to SEQ ID NO: 982

SEQ ID NO: 1130 - DNA, 39 bp, Homo sapiens; identical to SEQ ID NO: 983

SEQ ID NO: 1131 - DNA, 39 bp, Homo sapiens; identical to SEQ ID NO: 984

SEQ ID NO: 1132 - DNA, 39 bp, Homo sapiens; identical to SEQ ID NO: 985

SEQ ID NO: 1133 - DNA, 39 bp, Homo sapiens; identical to SEQ ID NO: 986

SEQ ID NO: 1134 - DNA, 39 bp, Homo sapiens; identical to SEQ ID NO: 987

SEQ ID NO: 1135 - DNA, 39 bp, Homo sapiens; identical to SEQ ID NO: 988

SEQ ID NO: 1136 - DNA, 39 bp, Homo sapiens; identical to SEQ ID NO: 534

SEQ ID NO: 1137 - DNA, 39 bp, Homo sapiens; identical to SEQ ID NO: 534

SEQ ID NO: 1138 - DNA, 39 bp, Homo sapiens; identical to SEQ ID NO: 991

SEQ ID NO: 1139 - DNA, 39 bp, Homo sapiens; identical to SEQ ID NO: 992

SEQ ID NO: 1140 - DNA, 39 bp, Homo sapiens; identical to SEQ ID NO: 538

SEQ ID NO: 1141 - DNA, 39 bp, Homo sapiens; identical to SEQ ID NO: 994

SEQ ID NO: 1142 - DNA, 39 bp, Homo sapiens; identical to SEQ ID NO: 539

SEQ ID NO: 1143 - DNA, 39 bp, Homo sapiens; identical to SEQ ID NO: 996

SEQ ID NO: 1144 - DNA, 39 bp, Homo sapiens; identical to SEQ ID NO: 997

SEQ ID NO: 1145 - DNA, 39 bp, Homo sapiens; identical to SEQ ID NO: 998

SEQ ID NO: 1146 - DNA, 39 bp, Homo sapiens; identical to SEQ ID NO: 999

SEQ ID NO: 1147 - DNA, 39 bp, Homo sapiens; identical to SEQ ID NO: 1000

SEQ ID NO: 1148 - DNA, 39 bp, Homo sapiens; identical to SEQ ID NO: 1001

SEQ ID NO: 1149 - DNA, 39 bp, Homo sapiens; identical to SEQ ID NO: 1002

SEQ ID NO: 1150 - DNA, 39 bp, Homo sapiens; identical to SEQ ID NO: 1003

SEQ ID NO: 1151 - DNA, 39 bp, Homo sapiens; identical to SEQ ID NO: 1003

SEQ ID NO: 1152 - DNA, 39 bp, Homo sapiens; identical to SEQ ID NO: 1005

SEQ ID NO: 1153 - DNA, 39 bp, Homo sapiens; identical to SEQ ID NO: 1005

SEQ ID NO: 1154 - DNA, 39 bp, Homo sapiens; identical to SEQ ID NO: 1007

SEQ ID NO: 1155 - DNA, 39 bp, Homo sapiens; identical to SEQ ID NO: 1008

SEQ ID NO: 1156 - DNA, 39 bp, Homo sapiens; identical to SEQ ID NO: 1009

SEQ ID NO: 1157 - DNA, 40 bp, Homo sapiens; identical to SEQ ID NO: 1010

SEQ ID NO: 1158 - DNA, 40 bp, Homo sapiens; identical to SEQ ID NO: 1010

SEQ ID NO: 1159 - DNA, 39 bp, Homo sapiens; identical to SEQ ID NO: 556

SEQ ID NO: 1160 - DNA, 39 bp, Homo sapiens; identical to SEQ ID NO: 1013

SEQ ID NO: 1161 - DNA, 39 bp, Homo sapiens; identical to SEQ ID NO: 1014

SEQ ID NO: 1162 - DNA, 39 bp, Homo sapiens; identical to SEQ ID NO: 1014

SEQ ID NO: 1163 - DNA, 39 bp, Homo sapiens; identical to SEQ ID NO: 559

SEQ ID NO: 1164 - DNA, 39 bp, Homo sapiens; identical to SEQ ID NO: 1017

SEQ ID NO: 1165 - DNA, 39 bp, Homo sapiens; identical to SEQ ID NO: 1018

SEQ ID NO: 1166 - DNA, 39 bp, Homo sapiens; identical to SEQ ID NO: 1018

SEQ ID NO: 1167 - DNA, 39 bp, Homo sapiens; identical to SEQ ID NO: 1020

SEQ ID NO: 1168 - DNA, 39 bp, Homo sapiens; identical to SEQ ID NO: 1021

SEQ ID NO: 1169 - DNA, 39 bp, Homo sapiens; identical to SEQ ID NO: 1022

SEQ ID NO: 1170 - DNA, 39 bp, Homo sapiens; identical to SEQ ID NO: 1023

SEQ ID NO: 1171 - DNA, 39 bp, Homo sapiens; identical to SEQ ID NO: 1024

SEQ ID NO: 1172 - DNA, 39 bp, Homo sapiens; identical to SEQ ID NO: 1025

SEQ ID NO: 1173 - DNA, 39 bp, Homo sapiens; identical to SEQ ID NO: 1026

SEQ ID NO: 1174 - DNA, 39 bp, Homo sapiens; identical to SEQ ID NO: 1027

SEQ ID NO: 1175 - DNA, 39 bp, Homo sapiens; identical to SEQ ID NO: 1028

SEQ ID NO: 1176 - DNA, 39 bp, Homo sapiens; identical to SEQ ID NO: 1029

SEQ ID NO: 1177 - DNA, 39 bp, Homo sapiens; identical to SEQ ID NO: 1030

SEQ ID NO: 1178 - DNA, 39 bp, Homo sapiens; identical to SEQ ID NO: 1031

SEQ ID NO: 1179 - DNA, 39 bp, Homo sapiens; identical to SEQ ID NO: 1032

SEQ ID NO: 1180 - DNA, 39 bp, Homo sapiens; identical to SEQ ID NO: 1033

SEQ ID NO: 1181 - DNA, 39 bp, Homo sapiens; identical to SEQ ID NO: 1034

SEQ ID NO: 1182 - DNA, 39 bp, Homo sapiens; identical to SEQ ID NO: 580

SEQ ID NO: 1183 - DNA, 39 bp, Homo sapiens; identical to SEQ ID NO: 580

SEQ ID NO: 1184 - DNA, 39 bp, Homo sapiens; identical to SEQ ID NO: 1037

SEQ ID NO: 1185 - DNA, 39 bp, Homo sapiens; identical to SEQ ID NO: 1038

SEQ ID NO: 1186 - DNA, 39 bp, Homo sapiens; identical to SEQ ID NO: 1039

SEQ ID NO: 1187 - DNA, 39 bp, Homo sapiens; identical to SEQ ID NO: 1040

SEQ ID NO: 1188 - DNA, 39 bp, Homo sapiens; identical to SEQ ID NO: 1041

SEQ ID NO: 1189 - DNA, 39 bp, Homo sapiens; identical to SEQ ID NO: 1041

SEQ ID NO: 1190 - DNA, 38 bp, Homo sapiens; identical to SEQ ID NO: 1043

SEQ ID NO: 1191 - DNA, 20 bp, Homo sapiens
gtgagccttacctgtggcaa

SEQ ID NO: 1192 - DNA, 22 bp, Homo sapiens
atccagacaggttatctgcacc

SEQ ID NO: 1193 - DNA, 25 bp, Homo sapiens
tactttacctccacaacttcaaacc

SEQ ID NO: 1194 - DNA, 20 bp, Homo sapiens
tcacatccatcagagatggc

SEQ ID NO: 1195 - DNA, 21 bp, Homo sapiens
ttcttcctcacaccttgcaga

SEQ ID NO: 1196 - DNA, 22 bp, Homo sapiens
aatgtgaagtttcaggggtctc

SEQ ID NO: 1197 - DNA, 29 bp, Homo sapiens
ttatcgccttaaaacatgtattgacttta

SEQ ID NO: 1198 - DNA, 20 bp, Homo sapiens
ttcctggatctcctctcccc

SEQ ID NO: 1199 - DNA, 32 bp, Homo sapiens
ggactttattcaagaataggagataagtacac

SEQ ID NO: 1200 - DNA, 25 bp, Homo sapiens
aaggcagctcctagtaaatgctcta

SEQ ID NO: 1201 - DNA, 18 bp, Homo sapiens
ttttcccagccccctctc

SEQ ID NO: 1202 - DNA, 26 bp, Homo sapiens
aaactccaggtgatctttaagtcaga

SEQ ID NO: 1203 - DNA, 18 bp, Homo sapiens
aatgccctgccctaagga

SEQ ID NO: 1204 - DNA, 25 bp, Homo sapiens
taaggaagacactcccaattctgtt

SEQ ID NO: 1205 - DNA, 18 bp, Homo sapiens
ggggctgctgcccataag

SEQ ID NO: 1206 - DNA, 27 bp, Homo sapiens
ataatgaccagcaaagaagttaaggat

SEQ ID NO: 1207 - DNA, 21 bp, Homo sapiens
aagaagtgggaggctcattga

SEQ ID NO: 1208 - DNA, 29 bp, Homo sapiens
ttctccttagatgccttagaagactaagt

SEQ ID NO: 1209 - DNA, 22 bp, Homo sapiens
cagactgggcaaaaattaacca

SEQ ID NO: 1210 - DNA, 34 bp, Homo sapiens
ctgttgtacgtattatagtttatttaactgacca

SEQ ID NO: 1211 - DNA, 26 bp, Homo sapiens
attgggtctgtttgtctctctaaagc

SEQ ID NO: 1212 - DNA, 19 bp, Homo sapiens
agagcccagagtgtgggag

SEQ ID NO: 1213 - DNA, 18 bp, Bacteriophage M13
tgtaaaacgacggccagt

SEQ ID NO: 1214 - DNA, 18 bp, Bacteriophage M13
caggaaacagctatgacc

SEQ ID NO: 1215 - DNA, 20 bp, Homo sapiens
agacttttccaatgaagagc

SEQ ID NO: 1216 - DNA, 24 bp, Bacteriophage M13
agcggataacaatttcacacagga

SEQ ID NO: 1217 - DNA, 21 bp, Homo sapiens
attttacggggtttgctgcat

SEQ ID NO: 1218 - DNA, 24 bp, Bacteriophage M13
cgccagggttttcccagtcacgac

SEQ ID NO: 1219 - DNA, 20 bp, Homo sapiens
gctcttcattggaaaagtct

(19) United States
(12) Patent Application Publication
(10) Pub. No.: US 2004/0033582 A1
(43) Pub. Date: Feb. 19, 2004

(54) Human single nucleotide polymorphisms

(76) Inventors: Manling-Ma Edmonds, Lawrenceville, PA (US); Lester Hui, Fairfax, VA (US); Mark Perrone, Boston, MA (US); James R. Powell, Lumberville, PA (US); Chandra S. Ramanathan, Wallingford, CT (US); Brian Swanson, Yardley, PA (US); Zenta Tsuchihashi, Skillman, NJ (US); Kim Zerba, New Hope, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/453,827

(22) Filed: Jun. 3, 2003

Related U.S. Application Data
(60) Provisional application No. 60/384,980, filed on Jun. 3, 2002.

(51) Int. Cl. C12Q 1/68; C07H 21/04; C12N 9/64; C12P 21/02; C12N 5/06

(52) U.S. Cl. 435/226; 435/69.1; 435/6; 435/320.1; 435/325; 536/23.2

(57) ABSTRACT

The invention provides polynucleotides and polypeptides corresponding to novel gene sequences associated with the incidence of cardiovascular disorders. The invention also provides polynucleotide fragments corresponding to the genomic and/or coding regions of these genes which comprise at least one polymorphic site per fragment. Allele-specific primers and probes which hybridize to these regions, and/or which comprise at least one polymorphic site are also provided. The polynucleotides, primers, and probes of the present invention are useful in phenotype correlations, paternity testing, medicine, and genetic analysis. Also provided are vectors, host cells, antibodies, and recombinant and synthetic methods for producing said polypeptides. The invention further relates to diagnostic and therapeutic methods for applying these novel polypeptides to the diagnosis, treatment, and/or prevention of various diseases and/or disorders, particularly cardiovascular diseases related to these polypeptides. The invention further relates to screening methods for identifying agonists and antagonists of the polynucleotides and polypeptides of the present invention.

This application claims benefit to provisional application U.S. Ser. No. 60/384,980 filed Jun. 3, 2002, under 35 U.S.C. 119(e). The entire teachings of the referenced applications are incorporated herein by reference.

### FIELD OF THE INVENTION

The invention provides polynucleotides and polypeptides corresponding to novel gene sequences associated with the incidence of cardiovascular diseases. The invention also provides polynucleotide fragments corresponding to the genomic and/or coding regions of these genes which comprise at least one polymorphic site per fragment. Allele-specific primers and probes which hybridize to these regions, and/or which comprise at least one polymorphic site are also provided. The polynucleotides, primers, and probes of the present invention are useful in phenotype correlations, paternity testing, medicine, and genetic analysis. Also provided are vectors, host cells, antibodies, and recombinant and synthetic methods for producing said polypeptides. The invention further relates to diagnostic and therapeutic methods for applying these novel polypeptides to the diagnosis, treatment, and/or prevention of various diseases and/or disorders, particularly cardiovascular diseases related to these polypeptides. The invention further relates to screening methods for identifying agonists and antagonists of the polynucleotides and polypeptides of the present invention.

### BACKGROUND OF THE INVENTION

The genomes of all organisms undergo spontaneous mutation in the course of their continuing evolution, generating variant forms of progenitor nucleic acid sequences (Gusella, Ann. Rev. Biochem., 55:831-854 (1986). The variant form may confer an evolutionary advantage or disadvantage relative to a progenitor form, or may be neutral. In some instances, a variant form confers a lethal disadvantage and is not transmitted to subsequent generations of the organism. In other instances, a variant form confers an evolutionary advantage to the species and is eventually incorporated into the DNA of many or most members of the species and effectively becomes the progenitor form. In many instances, both progenitor and variant form(s) survive and co- exist in a species population. The coexistence of multiple forms of a sequence gives rise to polymorphisms.

Several different types of polymorphism have been reported. A restriction fragment length polymorphism (RFLP) is a variation in DNA sequence that alters the length of a restriction fragment (Botstein et al., Am. J. Hum. Genet, 32:314-331 (1980). The restriction fragment length polymorphism may create or delete a restriction site, thus changing the length of the restriction fragment. RFLPs have been widely used in human and animal genetic analyses (see WO 90/13668; WO90/11369; Donis-Keller, Cell , 51:319-337 (1987); Lander et al., Genetics 121,85-99 (1989)). When a heritable trait can be linked to a particular RFLP, the presence of the RFLP in an individual can be used to predict the likelihood that the animal will also exhibit the trait.

Other polymorphisms take the form of short tandem repeats (STRs) that include tandem di-, tri- and tetra-nucleotide repeated motifs. These tandem repeats are also referred to as variable number tandem repeat (VNTR) polymorphisms. VNTRs have been used in identity and paternity analysis (U.S. Pat. No. 5,075,217; Annour et al., FEBSLett. 307, 113-115 (1992); Horn et al., WO 91/14003; Jeffreys, EP 370,719), and in a large number of genetic mapping studies.

Other polymorphisms take the form of single nucleotide variations between individuals of the same species. Such polymorphisms are far more frequent than RFLPs, STRs and VNTRs. Some single nucleotide polymorphisms (SNP) occur in protein-coding nucleic acid sequences (coding sequence SNP (cSNP)), in which case, one of the polymorphic forms may give rise to the expression of a defective or otherwise variant protein and, potentially, a genetic disease. Examples of genes in which polymorphisms within coding sequences give rise to genetic disease include ˜-globin (sickle cell anemia), apoE4 (Alzheimer's Disease), Factor V Leiden (thrombosis), and CFTR (cystic fibrosis). cSNPs can alter the codon sequence of the gene and therefore specify an alternative amino acid. Such changes are called “missense” when another amino acid is substituted, and “nonsense” when the alternative codon specifies a stop signal in protein translation. When the cSNP does not alter the amino acid specified the cSNP is called “silent”.

Other single nucleotide polymorphisms occur in noncoding regions. Some of these polymorphisms may also result in defective protein expression (e.g., as a result of defective splicing). Other single nucleotide polymorphisms have no phenotypic effects. Single nucleotide polymorphisms can be used in the same manner as RFLPs and VNTRs, but offer several advantages.

Single nucleotide polymorphisms occur with greater frequency and are spaced more uniformly throughout the genome than other forms of polymorphism. The greater frequency and uniformity of single nucleotide polymorphisms means that there is a greater probability that such a polymorphism will be found in close proximity to a genetic locus of interest than would be the case for other polymorphisms. The different forms of characterized single nucleotide polymorphisms are often easier to distinguish than other types of polymorphism (e.g., by use of assays employing allele-specific hybridization probes or primers).

Only a small percentage of the total repository of polymorphisms in humans and other organisms has been identified. The limited number of polymorphisms identified to date is due to the large amount of work required for their detection by conventional methods. For example, a conventional approach to identifying polymorphisms might be to sequence the same stretch of DNA in a population of individuals by dideoxy sequencing. In this type of approach, the amount of work increases in proportion to both the length of sequence and the number of individuals in a population and becomes impractical for large stretches of DNA or large numbers of persons.

Angiotensin converting enzyme (ACE) inhibitors are a class of therapeutic agents, which have been widely used for the treatment of hypertension (Brown N J 1998). Inhibition of ACE leads to a reduced concentration of angiotensin II, a key regulator of blood pressure. ACE inhibition also causes the increase of bradykinin, another ACE substrate, which is a vasodilator. This action also contributes to the reduction of blood pressure.

Vasopaptidase inhibitors are another class of therapeutic agents designed for hypertension treatment. Vasopeptidase inhibitors, such as Omapatrilat, inhibit both ACE and neutral endopeptidases (NEP) (Robl J A 1997; Coats 2000). NEP inhibition reduces the degradation of atrial natriuretic peptide (ANP), which also contributes to the decrease of blood pressure.

Angioedema is a relatively rare, but potentially life-threatening side effect associated with the administration of ACE inhibitors (Anderson M W 1990; Brown N J 1998; van Rijnsoever E W 1998; Agostoni A 1999). This side effect is believed to be a class effect directly caused by ACE inhibition, since it is observed with a variety of ACE inhibitors, and can develop after a long-term treatment, even though the majority of the cases occur within hours to days after the start of the treatment (Brown N J 1997; Schiller P I 1997; Agostoni A 1999). Angioedema has also been observed in vasopeptidase inhibitor treatment (Coats 2000). Angioedema has been noted to be more common in African Americans than in Caucasians in both ACE inhibitor and vasopeptidase inhibitor regimens, suggesting a genetic factor for susceptibility (Brown N J 1996; Brown N J 1998; Agostoni A 1999; Coats 2000). Hereditary forms of angioedema, which are independent of ACE inhibitors, is caused by a deficiency in C1 esterase inhibitor (Tosi 1998; Ebo D G 2000).

Bradykinin (BK) is a vasodilatory peptide generated from high molecular weight (HMW) kininogen through the action of serine proteases including tissue and plasma kallikreins (Barnes 1997). Two types of bradykinin receptors, B1 and B2 have been identified, of which the B2 receptors are, in general, constitutively expressed, while the B1 receptors are inducibly expressed (Marceau F 1997; Marceau, Hess et al. 1998; Marceau F 1998). Lys-des-Arg¹⁰ bradykinin (des-Arg¹⁰ kallidin),Lys-bradykinin (kallidin) is another peptide derived from kininogen through the action of tissue kallikrein. Both bradykinin and kallidin are substrates of kininase I (generic name for carboxypeptidases which act on bradykinin including carboxypeptidase M, carboxypeptidase N, and carboxypeptidase U), which converts them into des-Arg⁹ bradykinin and des-Arg¹⁰ kallidin, respectively. Both des-Arg⁹ bradykinin and des-Arg¹⁰ kallidin are much more potent effectors for the B1 receptor than bradykinin and kallidin themselves. Both des-Arg⁹ bradykinin and des-Arg¹⁰ kallidin are inactivated by aminopeptidase P as well as by ACE and NEP (Marceau F 1997; Marceau F 1998; Marceau F 1999). Some of the actions of bradykinin are mediated through NK1 tachykinin receptor after induction of substance P (Marceau, Hess et al. 1998).

Genetic polymorphisms in members of the bradykinin pathway, in addition to other proteins described herein, may cause alterations in the level of bradykinin or its related peptides, or may affect downstream signal transduction. Such polymorphisms may genetically predispose certain individuals to an increased risk of developing angioedema. Such polymorphisms are expected to show a significant difference in allele frequency between healthy individuals and angioedema subjects. Genotypes of such polymorphisms can predict each individual's susceptibility to angioedema, and thus will be useful in identifying a group of high risk individuals that may be subject to modified ACE inhibitor or vasopeptidase inhibitor treatment regimens. Alternatively, the identification of such a group may preclude one or more individuals within said group from being administered an ACE inhibitor or vasopeptidase inhibitor.

### SUMMARY OF THE INVENTION

The present invention pertains to single nucleotide polymorphisms which can predispose individuals to disease, by resequencing large numbers of genes in a large number of individuals. Various genes from a number of individuals have been resequenced as described herein, and SNPs in these genes have been genotyped and are thought to be associated with increased susceptibility to angioedema (Table I, II, and III). Some of these SNPs are cSNPs (coding SNPs) which specify a different amino acid sequence (described as “missense” under the ‘Mutation Type’ column of the Tables); some of the SNPs are silent cSNPs (shown as mutation type “silent” under the ‘Mutation Type’ column of the Tables), and some of these cSNPs may specify a stop signal in protein translation. Some of the identified SNPs were located in non-coding regions (described as “non-CDS” in the ‘Mutation Type’ column in the Tables).

The invention relates to a nucleic acid molecule which comprises a single nucleotide polymorphism at a specific location. In a particular embodiment the invention relates to the variant allele of a gene or polynucleotide having a single nucleotide polymorphism, which variant allele differs from a reference allele by one nucleotide at the site(s) identified in Table I, II, III, or elsewhere herein. Complements of these nucleic acid segments are also provided. The segments can be DNA or RNA, and can be double- or single-stranded. Segments can be, for example, 5-10,5-15, 10-20,5-25,10-30, 10-50 or 10-100 bases long. In another embodiment, the invention relates to the reference or wild type allele of a gene or polynucleotide having a single nucleotide polymorphism, which reference or wild type allele differs from a variant allele by one nucleotide at the site(s) identified in Table I, II, III or elsewhere herein. Complements of these nucleic acid segments are also provided. The segments can be DNA or RNA, and can be double- or single-stranded. Segments can be, for example, 5-10,5-15, 10-20,5-25,10-30, 10-50 or 10-100 bases long.

The invention further provides variant and reference allele-specific oligonucleotides that hybridize to a nucleic acid molecule comprising a single nucleotide polymorphism or to the complement of the nucleic acid molecule. These oligonucleotides can be probes or primers.

The invention further provides oligonucleotides that may be used to amplify across a single nucleotide polymorphic site of the present invention. The invention further provides oligonucleotides that may be used to sequence said amplified sequence. The invention further provides a method of analyzing a nucleic acid from a DNA sample using said amplification and sequencing primers to assess whether said sample contains the reference or variant base (allele) at the polymorphic site, comprising the steps of amplifying a sequence using appropriate PCR primers for amplifying across a polymorphic site, sequencing the resulting amplified product using appropriate sequencing primers to sequence said product, and determining whether the variant or reference base is present at the polymorphic site. The invention further provides a method of analyzing a nucleic acid from DNA sample(s) from various ethnic populations using said amplification and sequencing primers to assess whether said sample(s) contain the reference or variant base (allele) at the polymorphic site in an effort to identify populations at risk of developing angiodema upon administration of an ACE inhibitor and/or vasopeptidase inhibitor and/or neutral endopeptidase (NEP) inhibitor, comprising the steps of amplifying a sequence using appropriate PCR primers for amplifying across a polymorphic site, sequencing the resulting amplified product using appropriate sequencing primers to sequence said product, and determining whether the variant or reference base is present at the polymorphic site, and optionally determining the statistical association between either the reference or variant allele at the polymorphic site(s) to the incidence of angioedema.

The invention further provides oligonucleotides that may be used to genotype DNA sample(s) to assess whether said sample(s) contain the reference or variant base (allele) at the polymorphic site(s). The invention provide a method of using oligonucleotides that may be used to genotype a DNA sample to assess whether said sample contains the reference or variant base (allele) at the polymorphic site comprising the steps of amplifying a sequence using appropriate PCR primers for amplifying across a polymorphic site, subjecting the product of said amplification to a genetic bit analysis (GBA) reaction, and analyzing the result.

The invention provides a method of using oligonucleotides that may be used to genotype DNA sample(s) to identify individual(s) that may be at risk of developing angioedema upon administration of an ACE inhibitor and/or vasopeptidase inhibitor and/or neutral endopeptidase (NEP) inhibitor to assess whether said sample(s) contains the reference or variant base (allele) at the polymorphic site(s) comprising the steps of amplifying a sequence using appropriate PCR primers for amplifying across a polymorphic site, subjecting the product of said amplification to a genetic bit analysis (GBA) reaction, analyzing the result, and optionally determining the statistical association between either the reference or variant allele at the polymorphic site(s) to the incidence of angioedema..

The invention provides a method of using oligonucleotides that may be used to genotype DNA sample(s) to identify ethnic population(s) that may be at risk of developing angioedema upon administration of an ACE inhibitor and/or vasopeptidase inhibitor and/or neutral endopeptidase (NEP) inhibitor to assess whether said sample(s) contain the reference or variant base (allele) at the polymorphic site comprising the steps of amplifying a sequence using appropriate PCR primers for amplifying across a polymorphic site, subjecting the product of said amplification to a genetic bit analysis (GBA) reaction, analyzing the result, and optionally determining the statistical association between either the reference or variant allele at the polymorphic site(s) to the incidence of angioedema.

The invention further provides a method of analyzing a nucleic acid from an individual. The method allows the determination of whether the reference or variant base is present at any one, or more, of the polymorphic sites shown in Table I, II, III or elsewhere herein. Optionally, a set of bases occupying a set of the polymorphic sites shown in Table I, II, III or elsewhere herein, is determined. This type of analysis can be performed on a number of individuals, who are also tested (previously, concurrently or subsequently) for the presence of a disease phenotype. The presence or absence of disease phenotype is then correlated with a base or set of bases present at the polymorphic site or sites in the individuals tested.

Thus, the invention further relates to a method of predicting the presence, absence, likelihood of the presence or absence, or severity of a particular phenotype or disorder associated with a particular genotype. The method comprises obtaining a nucleic acid sample from an individual and determining the identity of one or more bases (nucleotides) at specific (e.g., polymorphic) sites of nucleic acid molecules described herein, wherein the presence of a particular base at that site is correlated with a specified phenotype or disorder, thereby predicting the presence, absence, likelihood of the presence: or absence, or severity of the phenotype or disorder in the individual, wherein the phenotype or disorder is preferably a cardiovascular disease, and more preferably either angioedema or an angioedema-like disorder.

The invention further relates to polynucleotides having one or more variant alleles. The invention also relates to said polynucleotides lacking a start codon. The invention further relates to polynucleotides of the present invention containing one or more variant alleles wherein said polynucleotides encode a polypeptide of the present invention. The invention relates to polypeptides of the present invention containing one or more variant amino acids encoded by one or more variant alleles.

The present invention relates to antisense oligonucleotides corresponding to the polynucleotides of the present invention. Preferably, such antisense oligonucleotides are capable of discriminating against the reference or variant allele of the polynucleotide, preferably at one or more polymorphic sites of said polynucleotide.

The present invention relates to antibodies directed against the polypeptides of the present invention. Preferably, such antibodies are capable of discriminating against the reference or variant allele of the polypeptide, preferably at one or more polymorphic sites of said polynucleotide.

The present invention also relates to recombinant vectors, which include the isolated nucleic acid molecules of the present invention, and to host cells containing the recombinant vectors, as well as to methods of making such vectors and host cells, in addition to their use in the production of polypeptides or peptides provided herein using recombinant techniques. Synthetic methods for producing the polypeptides and polynucleotides of the present invention are provided. Also provided are diagnostic methods for detecting diseases, disorders, and/or conditions related to the polypeptides and polynucleotides provided herein, and therapeutic methods for treating such diseases, disorders, and/or conditions. The invention further relates to screening methods for identifying binding partners of the polypeptides.

The invention further provides an isolated polypeptide having an amino acid sequence encoded by a polynucleotide described herein.

The invention further relates to the identification of SNPs that have been determined to represent a random sampling of SNPs throughout the genome of a DNA sample, or sample(s), such as the SNPs provided in Tables I, II, or III herein.

The invention further relates to the use of such randomly distributed SNPs as a means of increasing the accuracy of ethnic assignments for genomic control DNA sample(s) of the present invention. The increased ethnic accuracy of such genomic controls results in an increased statistical confidence in the angioedema association data obtained for any one or more SNPs of the present invention.

The invention further relates to the use of such genomic control SNPs for clustering individuals to confirm known gene pool/racial/ethnic groups or to reveal cryptic SNPs in a DNA sample(s).

The invention relates to a method of analyzing at least one nucleic acid sample, comprising the steps of (1) obtaining said nucleic acid sample from one or more individuals; and (2) determining the nucleic acid sequence at one or more polymorphic positions in a gene encoding a protein selected from the group consisting of ANPEP, C1S, GUCY1A2, KLK1, MEP1B, XPNPEP2, or XPNPEPL.

The invention further relates to a method of analyzing at least one nucleic acid sample, further comprising the steps of (3) testing each individual for the presence of a disease phenotype; and (4) correlating the presence of the disease phenotype with the sequence at said one or more polymorphic positions. Preferably wherein the disease phenotype is angioedema or an angioedema-like disorder.

The invention further relates to a method of analyzing at least one nucleic acid sample, wherein said one or more polymorphic positions of said nucleic acid sequence is a polymorphic position specified in Table I, II, or III for said gene.

The invention further relates to a method of constructing haplotypes using the isolated nucleic acids referred to in Table I, II, or III, comprising the step of grouping at least two said nucleic acids.

The invention further relates to a method of constructing haplotypes further comprising the step of using said haplotypes to identify an individual for the presence of a disease phenotype, and correlating the presence of the disease phenotype with said haplotype, preferably wherein the disease phenotype is angioedema or an angioedema-like disorder.

The invention further relates to a method for identifying an individual at risk of developing a disorder upon administration of a pharmaceutically acceptable amount of an ACE inhibitor and/or vasopeptidase inhibitor comprising the steps of (a) obtaining nucleic acid sample(s) from said individual; (b) amplifying one or more sequences from said sample(s) using appropriate PCR primers for amplifying across at least one polymorphic position; (c) comparing said at least one polymorphic position with a known data set; and (d) determining whether the result correlates with an increased or decreased risk for developing a disorder, preferably wherein the disease phenotype is angioedema or an angioedema-like disorder..

The invention further relates to a library of nucleic acids, each of which comprises one or more polymorphic positions within a gene encoding a human protein selected from the group consisting of ANPEP, C1S, GUCY1A2, KLK1, MEP1B, XPNPEP2, or XPNPEPL, wherein said polymorphic positions are selected from a group consisting of the polymorphic positions provided in Table I, II, or III.

The invention further relates to a library of nucleic acids, wherein the sequence at said polymorphic position is selected from the group consisting of the sequences provided in Table I, II, or III.

The invention further relates to a library of nucleic acids, wherein the sequence at said polymorphic position is selected from the group consisting of the sequences provided in Table I, II, or III, wherein said library of isolated sequences represents the complimentary sequence of said sequences.

The invention further relates to a kit for identifying an individual at risk of developing a disorder upon administration of a pharmaceutically acceptable amount of an ACE inhibitor and/or vasopeptidase inhibitor, said kit comprising sequencing primers, and sequencing reagents, wherein said primers are primers that hybridize to at least one polymorphic position in a human gene selected from the group consisting of ANPEP, C1S, GUCY1A2, KLK1, MEP1B, XPNPEP2, or XPNPEPL.

The invention further relates to a kit for identifying an individual at risk of developing a disorder upon administration of a pharmaceutically acceptable amount of an ACE inhibitor and/or vasopeptidase inhibitor, wherein said polymorphic positions are selected from a group consisting of the polymorphic positions provided in Table I, II, or III.

The invention further relates to a kit for identifying an individual at risk of developing a disorder upon administration of a pharmaceutically acceptable amount of an ACE inhibitor and/or vasopeptidase inhibitor, wherein said polymorphic positions are selected from a group consisting of the polymorphic positions provided in Table I, II, or III, wherein said primer(s) hybridizes immediately adjacent to said polymorphic positions, or wherein said primer(s) hybridizes to said polymorphic positions such that the central position of the primer aligns with the polymorphic position of said gene.

The invention further relates to a method for identifying an individual at risk of developing a disorder upon administration of a pharmaceutically acceptable amount of an ACE inhibitor and/or vasopeptidase inhibitor comprising the steps of (a) obtaining a nucleic acid sample(s) from said individual; (b) determining the nucleotide present at least one polymorphic position, (c) comparing said at least one polymorphic position with a known data set; and (d) determining whether the result correlates with an increased or decreased risk for developing a disorder, preferably wherein the disorder is angioedema or an angioedema-like disorder.

The invention further relates to a method for predicting the likelihood that an individual will be diagnosed as being at risk of developing an angioedema or angioedema-like disorder upon administration of a pharmaceutically acceptable amount of an ACE inhibitor and/or vasopeptidase inhibitor comprising the steps of (a) obtaining a nucleic acid sample(s) from am individual to be assessed; and (b) determining the nucleotide present at one or more polymorphic position(s) of a gene selected from the group consisting of: SEQ ID NO:883, 885, 887, 889, 891, 893, and 895, wherein the presence of the reference nucleotide at the one or more polymorphic position(s) indicates that the individual has a lower likelihood of being diagnosed as at risk of developing an angioedema or angioedema-like disorder upon administration of a pharmaceutically acceptable amount of an ACE inhibitor and/or vasopeptidase inhibitor as compared to an individual having an alternate allele at said polymorphic position(s).

The invention further relates to a method for predicting the likelihood that an individual will be diagnosed as being at risk of developing an angioedema or angioedema-like disorder upon administration of a pharmaceutically acceptable amount of an ACE inhibitor and/or vasopeptidase inhibitor comprising the steps of (a) obtaining a nucleic acid sample(s) from am individual to be assessed; and (b) determining the nucleotide present at one or more polymorphic position(s) of a gene selected from the group consisting of: SEQ ID NO:883, 885, 887, 889, 891, 893, and 895, wherein the presence of the reference nucleotide at the one or more polymorphic position(s) indicates that the individual has a higher likelihood of being diagnosed as at risk of developing an angioedema or angioedema-like disorder upon administration of a pharmaceutically acceptable amount of an ACE inhibitor and/or vasopeptidase inhibitor as compared to an individual having an alternate allele at said polymorphic position(s).

The invention further relates to a method for predicting the likelihood that an individual will be diagnosed as being at risk of developing an angioedema or angioedema-like disorder upon administration of a pharmaceutically acceptable amount of an ACE inhibitor and/or vasopeptidase inhibitor comprising the steps of (a) obtaining a nucleic acid sample(s) from am individual to be assessed; and (b) determining the nucleotide present at one or more polymorphic position(s) of a gene selected from the group consisting of: SEQ ID NO:883, 885, 887, 889, 891, 893, and 895, wherein the presence of the alternate nucleotide at the one or more polymorphic position(s) indicates that the individual has a lower likelihood of being diagnosed as at risk of developing an angioedema or angioedema-like disorder upon administration of a pharmaceutically acceptable amount of an ACE inhibitor and/or vasopeptidase inhibitor as compared to an individual having a reference nucleotide at said polymorphic position(s).

The invention further relates to a method for predicting the likelihood that an individual will be diagnosed as being at risk of developing an angioedema or angioedema-like disorder upon administration of a pharmaceutically acceptable amount of an ACE inhibitor and/or vasopeptidase inhibitor comprising the steps of (a) obtaining a nucleic acid sample(s) from am individual to be assessed; and (b) determining the nucleotide present at one or more polymorphic position(s) of a gene selected from the group consisting of: SEQ ID NO:883, 885, 887, 889, 891, 893, and 895, wherein the presence of the alternate nucleotide at the one or more polymorphic position(s) indicates that the individual has a higher likelihood of being diagnosed as at risk of developing an angioedema or angioedema-like disorder upon administration of a pharmaceutically acceptable amount of an ACE inhibitor and/or vasopeptidase inhibitor as compared to an individual having a reference nucleotide at said polymorphic position(s).

The invention further relates to a method for predicting the likelihood that an individual will be diagnosed as being at risk of developing an angioedema or angioedema-like disorder upon administration of a pharmaceutically acceptable amount of an ACE inhibitor and/or vasopeptidase inhibitor comprising the steps of (a) obtaining a nucleic acid sample(s) from am individual to be assessed; and (b) determining the nucleotide present at one or more polymorphic position(s) of a gene selected from the group consisting of: nucleotide position 558, 643, 982, 1122, 1136, 1879 of SEQ ID NO:883; nucleotide position 2733, 2664, 2553, 2519, 2505, 1929, 1928, 1227, 1083, 1074, 1052, 747, 474 of SEQ ID NO:885; nucleotide position 196, 394, 838, 1022, 1268, 1315, 1740, 1741, 1999, 2130 of SEQ ID NO:887; nucleotide position 1773, 225 of SEQ ID NO:889; nucleotide position 603 of SEQ ID NO:891; nucleotide position 2172 of SEQ ID NO:893; nucleotide position 2169, 825, and 822 of SEQ ID NO:895, wherein the presence of the reference nucleotide at the one or more polymorphic position(s) as provided in Table I indicates that the individual has a lower likelihood of being diagnosed as at risk of developing an angioedema or angioedema-like disorder upon administration of a pharmaceutically acceptable amount of an ACE inhibitor and/or vasopeptidase inhibitor as compared to an individual having an alternate allele at said polymorphic position(s).

The invention further relates to a method for predicting the likelihood that an individual will be diagnosed as being at risk of developing an angioedema or angioedema-like disorder upon administration of a pharmaceutically acceptable amount of an ACE inhibitor and/or vasopeptidase inhibitor comprising the steps of (a) obtaining a nucleic acid sample(s) from am individual to be assessed; and (b) determining the nucleotide present at one or more polymorphic position(s) of a gene selected from the group consisting of: nucleotide position 558, 643, 982, 1122, 1136, 1879 of SEQ ID NO:883; nucleotide position 2733, 2664, 2553, 2519, 2505, 1929, 1928, 1227, 1083, 1074, 1052, 747, 474 of SEQ ID NO:885; nucleotide position 196, 394, 838, 1022, 1268, 1315, 1740, 1741, 1999, 2130 of SEQ ID NO:887; nucleotide position 1773, 225 of SEQ ID NO:889; nucleotide position 603 of SEQ ID NO:891; nucleotide position 2172 of SEQ ID NO:893; nucleotide position 2169, 825, and 822 of SEQ ID NO:895, wherein the presence of the reference nucleotide at the one or more polymorphic position(s) as provided in Table I indicates that the individual has a higher likelihood of being diagnosed as at risk of developing an angioedema or angioedema-like disorder upon administration of a pharmaceutically acceptable amount of an ACE inhibitor and/or vasopeptidase inhibitor as compared to an individual having an alternate allele at said polymorphic position(s).

The invention further relates to a method for predicting the likelihood that an individual will be diagnosed as being at risk of developing an angioedema or angioedema-like disorder upon administration of a pharmaceutically acceptable amount of an ACE inhibitor and/or vasopeptidase inhibitor comprising the steps of (a) obtaining a nucleic acid sample(s) from am individual to be assessed; and (b) determining the nucleotide present at one or more polymorphic position(s) of a gene selected from the group consisting of: nucleotide position 558, 643, 982, 1122, 1136, 1879 of SEQ ID NO:883; nucleotide position 2733, 2664, 2553, 2519, 2505, 1929, 1928, 1227, 1083, 1074, 1052, 747, 474 of SEQ ID NO:885; nucleotide position 196, 394, 838, 1022, 1268, 1315, 1740, 1741, 1999, 2130 of SEQ ID NO:887; nucleotide position 1773, 225 of SEQ ID NO:889; nucleotide position 603 of SEQ ID NO:891; nucleotide position 2172 of SEQ ID NO:893; nucleotide position 2169, 825, and 822 of SEQ ID NO:895, wherein the presence of the alternate nucleotide at the one or more polymorphic position(s) as provided in Table I indicates that the individual has a lower likelihood of being diagnosed as at risk of developing an angioedema or angioedema-like disorder upon administration of a pharmaceutically acceptable amount of an ACE inhibitor and/or vasopeptidase inhibitor as compared to an individual having an alternate allele at said polymorphic position(s).

The invention further relates to a method for predicting the likelihood that an individual will be diagnosed as being at risk of developing an angioedema or angioedema-like disorder upon administration of a pharmaceutically acceptable amount of an ACE inhibitor and/or vasopeptidase inhibitor comprising the steps of (a) obtaining a nucleic acid sample(s) from am individual to be assessed; and (b) determining the nucleotide present at one or more polymorphic position(s) of a gene selected from the group consisting of: nucleotide position 558, 643, 982, 1122, 1136, 1879 of SEQ ID NO:883; nucleotide position 2733, 2664, 2553, 2519, 2505, 1929, 1928, 1227, 1083, 1074, 1052, 747, 474 of SEQ ID NO:885; nucleotide position 196, 394, 838, 1022, 1268, 1315, 1740, 1741, 1999, 2130 of SEQ ID NO:887; nucleotide position 1773, 225 of SEQ ID NO:889; nucleotide position 603 of SEQ ID NO:891; nucleotide position 2172 of SEQ ID NO:893; nucleotide position 2169, 825, and 822 of SEQ ID NO:895, wherein the presence of the alternate nucleotide at the one or more polymorphic position(s) as provided in Table I indicates that the individual has a higher likelihood of being diagnosed as at risk of developing an angioedema or angioedema-like disorder upon administration of a pharmaceutically acceptable amount of an ACE inhibitor and/or vasopeptidase inhibitor as compared to an individual having an alternate allele at said polymorphic position(s).

The invention further relates to a method for predicting the likelihood that an individual will be diagnosed as being at risk of developing an angioedema or angioedema-like disorder upon administration of a pharmaceutically acceptable amount of an ACE inhibitor and/or vasopeptidase inhibitor wherein said individual is an individual at risk for developing an angioedema or angioedema-like disorder upon administration of a pharmaceutically acceptable amount of an ACE inhibitor and/or vasopeptidase inhibitor.

The invention further relates to a method for genotyping an individual comprising the steps of (a) obtaining a nucleic acid sample(s) from said individual; (b) determining the nucleotide present at least one polymorphic position, and (c) comparing said at least one polymorphic position with a known data set.

### BRIEF DESCRIPTION OF THE FIGURES/DRAWINGS

FIGS. 1A-C show the polynucleotide sequence (SEQ ID NO: 883) and deduced amino acid sequence (SEQ ID NO: 884) of the human complement component 1, s subcomponent protein, CIS (Genbank Accession No: NM_{—}001734.1) comprising, or alternatively consisting of, one or more of the predicted polynucleotide polymorphic loci, in addition to, the encoded polypeptide polymorphic loci of the present invention for this particular protein, which include but are not limited to the following polynucleotide polymorphisms: C1S-G558A (SNP_ID: AE111s17), C1S-C643T (SNP_ID: AE111s18), C1S-T982A (SNP_ID: AE111s20), C1S-C1122T (SNP_ID: AE111s21), C1S-G1136A (SNP_ID: AE111s22), and/or C1S-C1879T (SNP_ID: AE111s8); and polypeptide polymorphism—C1S-R119H (SNP_ID: AE111s17), C1S-N260K (SNP_ID: AE111s20), C1S-A307V (SNP_ID: AE111s21), and/or C1S-V3121 (SNP_ID: AE111s22). The standard one-letter abbreviation for amino acids is used to illustrate the deduced amino acid sequence. The polynucleotide sequence contains a sequence of 2647 nucleotides (SEQ ID NO:883), encoding a polypeptide of 688 amino acids (SEQ ID NO:884). The polynucleotide polymorphic sites are represented by an “N”, in bold. The polypeptide polymorphic sites are represented by an “X”, in bold. The present invention encompasses the polynucleotide at nucleotide position 558 as being either a “G” or an “A” (SEQ ID NO:885), the polynucleotide at nucleotide position 643 as being either a “C” or a “T”, the polynucleotide at nucleotide position 982 as being either a “T” or an “A”, the polynucleotide at nucleotide position 1122 as being either a “C” or a “T”, the polynucleotide at nucleotide position 1136 as being either a “G” or an “A”, and the polynucleotide at nucleotide position 1879 as being either a “C” or a “T” of FIGS. 1A-C (SEQ ID NO:289), in addition to any combination thereof. The present invention also encompasses the polypeptide at amino acid position 119 as being either an “Arg” or an “His”, the polypeptide at amino acid position 260 as being either an “Asn” or a “Lys”, the polypeptide at amino acid position 307 as being either a “Ala” or a “Val”, and the polypeptide at amino acid position 312 as being either a “Val” or a “Ile” of FIGS. 1A-C (SEQ ID NO:884).

FIGS. 2A-D show the polynucleotide sequence (SEQ ID NO:885) and deduced amino acid sequence (SEQ ID NO:886) of the human alanyl (membrane) aminopeptidase protein, ANPEP (Genbank Accession No: NM_{—}001150.1) comprising, or alternatively consisting of, one or more of the predicted polynucleotide polymorphic loci, in addition to, the encoded polypeptide polymorphic loci of the present invention for this particular protein, which include but are not limited to the following polynucleotide polymorphisms: ANPEP-T2733C (SNP_ID: AE112s36), ANPEP-C2664A (SNP_ID: AE112s38), ANPEP-C2553T (SNP_ID: AE112s33), ANPEP-C2519T (SNP_ID: AE112s32), ANPEP-C2505T (SNP_ID: AE112s30), ANPEP-A1929G (SNP_ID: AE112s18), ANPEP-T1928A (SNP_ID: AE112s19), ANPEP-C1227T (SNP_ID: AE112s68), ANPEP-G1083A (SNP_ID: AE112s63), ANPEP-G1074T (SNP_ID: AE112s62), ANPEP-C1052T (SNP_ID: AE112s61), ANPEP-T747A (SNP_ID: AE112s52), and/or ANPEP-C474T (SNP_ID: AE112s15); and polypeptide polymorphism—ANPEP-A800V (SNP_ID: AE112s32), ANPEP-I603M (SNP_ID: AE112s18), ANPEP-I603K (SNP_ID: AE112s19), and/or ANPEP-A311 V (SNP_ID: AE112s61). The standard one-letter abbreviation for amino acids is used to illustrate the deduced amino acid sequence. The polynucleotide sequence contains a sequence of 3494 nucleotides (SEQ ID NO:885), encoding a polypeptide of 967 amino acids (SEQ ID NO:886). The polynucleotide polymorphic sites are represented by an “N”, in bold. The polypeptide polymorphic sites are represented by an “X”, in bold. The present invention encompasses the polynucleotide at nucleotide position 2733 as being either a “T” or a “C”, the polynucleotide at nucleotide position 2664 as being either a “C” or an “A”, the polynucleotide at nucleotide position 2553 as being either a “C” or a “T”, the polynucleotide at nucleotide position 2519 as being either a “C” or a “T”, the polynucleotide at nucleotide position 2505 as being either a “C” or a “T”, the polynucleotide at nucleotide position 1929 as being either an “A” or a “G”, the polynucleotide at nucleotide position 1928 as being either a “T” or an “A”, the polynucleotide at nucleotide position 1227 as being either a “C” or a “T”, the polynucleotide at nucleotide position 1083 as being either a “G” or an “A”, the polynucleotide at nucleotide position 1074 as being either a “G” or a “T”, the polynucleotide at nucleotide position 1052 as being either a “C” or a “T”, the polynucleotide at nucleotide position 747 as being either a “T” or an “A”, and the polynucleotide at nucleotide position 474 as being either a “C” or a “T” of FIGS. 2A-D (SEQ ID NO:885), in addition to any combination thereof. The present invention also encompasses the polypeptide at amino acid position 800 as being either an “Ala” or a “Val”, the polypeptide at amino acid position 603 as being either an “Ile” or a “Met”, the polypeptide at amino acid position 603 as being either a “Ile” or a “Lys”, and the polypeptide at amino acid position 311 as being either a “Ala” or a “Val” of FIGS. 2A-D (SEQ ID NO:886). deduced amino acid sequence (SEQ ID NO:888) of the human meprin A, beta protein, MEP1B (Genbank Accession No: NM_{—}005925.1) comprising, or alternatively consisting of, one or more of the predicted polynucleotide polymorphic loci, in addition to, the encoded polypeptide polymorphic loci of the present invention for this particular protein, which include but are not limited to the following polynucleotide polymorphisms: MEP1B-G196A (SNP_ID: AE113s34), MEP1B-A394G (SNP_ID: AE113s38), MEP1B-A838G (SNP_ID: AE113s43), MEP1B-G1022A (SNP_ID: AE113s6), MEP1B-T1268A (SNP_ID: AE113s9), MEP1B-G1315A (SNP_ID: AE113s8), MEP1B-A1740C (SNP_ID: AE113s18), MEP1B-C1741T (SNP_ID: AE113s14), MEP1B-C1999T (SNP_ID: AE113s24), and/or MEP1B-T2130C (SNP_ID: AE113s25); and polypeptide polymorphism—MEP1B-I115M (SNP_ID: AE113s38), MEP1B-V326M (SNP_ID: AE113s6), MEP1B-S408T (SNP_ID: AE113s9), MEP1B-H565P (SNP_ID: AE113s18), and/or MEP1B-L695P (SNP_ID: AE113s25). The standard one-letter abbreviation for amino acids is used to illustrate the deduced amino acid sequence. The polynucleotide sequence contains a sequence of 2236 nucleotides (SEQ ID NO:887), encoding a polypeptide of 699 amino acids (SEQ ID NO:888). The polynucleotide polymorphic sites are represented by an “N”, in bold. The polypeptide polymorphic sites are represented by an “X”, in bold. The present invention encompasses the polynucleotide at nucleotide position 196 as being either a “G” or an “A”, the polynucleotide at nucleotide position 394 as being either an “A” or a “G”, the polynucleotide at nucleotide position 838 as being either an “A” or a “G”, the polynucleotide at nucleotide position 1022 as being either a “G” or an “A”, the polynucleotide at nucleotide position 1268 as being either a “T” or an “A”, the polynucleotide at nucleotide position 1315 as being either a “G” or an “A”, the polynucleotide at nucleotide position 1740 as being either an “A” or a “C”, the polynucleotide at nucleotide position 1741 as being either a “C” or a “T”, the polynucleotide at nucleotide position 1999 as being either a “C” or a “T”, and/or the polynucleotide at nucleotide position 2130 as being either a “T” or a “C” of FIGS. 3A-C (SEQ ID NO:887), in addition to any combination thereof. The present invention also encompasses the polypeptide at amino acid position 115 as being either an “Ile” or a “Met”, the polypeptide at amino acid position 326 as being either an “Val” or a “Met”, the polypeptide at amino acid position 408 as being either a “Ser” or a “Thr”, the polypeptide at amino acid position 565 as being either a “His” or a “Pro”, and the polypeptide at amino acid position 695 as being either a “Leu” or a “Pro” of FIGS. 3A-C (SEQ ID NO:888).

FIGS. 4A-B show the polynucleotide sequence (SEQ ID NO:889) and deduced amino acid sequence (SEQ ID NO:890) of the human X-prolyl aminopeptidase (aminopeptidase P)-like protein, XPNPEPL (Genbank Accession No: NM_{—}006523.1) comprising, or alternatively consisting of, one or more of the predicted polynucleotide polymorphic loci, in addition to, the encoded polypeptide polymorphic loci of the present invention for this particular protein, which include but are not limited to the following polynucleotide polymorphisms: XPNPEPL-C1773G (SNP_ID: AE114s30), and/or XPNPEPL-C225T (SNP_ID: AE114s33). The standard one-letter abbreviation for amino acids is used to illustrate the deduced amino acid sequence. The polynucleotide sequence contains a sequence of 3494 nucleotides (SEQ ID NO:889), encoding a polypeptide of 967 amino acids (SEQ ID NO:890). The polynucleotide polymorphic sites are represented by an “N”, in bold. The polypeptide polymorphic sites are represented by an “X”, in bold. The present invention encompasses the polynucleotide at nucleotide position 1773 as being either a “C” or a “G, and/or the polynucleotide at nucleotide position 225 as being either a “C” or a “T” of FIGS. 4A-B (SEQ ID NO:889), in addition to any combination thereof.

FIG. 5 shows the polynucleotide sequence (SEQ ID NO:891) and deduced amino acid sequence (SEQ ID NO:892) of the human kallikrein 1 protein, KLK1 (Genbank Accession No: NM_{—}002257.1) comprising, or alternatively consisting of, one or more of the predicted polynucleotide polymorphic loci, which include but are not limited to the following polynucleotide polymorphisms: KLK1-C603T (SNP_ID: AE115s11). The standard one-letter abbreviation for amino acids is used to illustrate the deduced amino acid sequence. The polynucleotide sequence contains a sequence of 871 nucleotides (SEQ ID NO:891), encoding a polypeptide of 261 amino acids (SEQ ID NO:892). The polynucleotide polymorphic sites are represented by an “N”, in bold. The present invention encompasses the polynucleotide at nucleotide position 603 as being either a “C” or a “T” of FIG. 5 (SEQ ID NO:891), in addition to any combination thereof.

FIGS. 6A-D show the polynucleotide sequence (SEQ ID NO:893) and deduced amino acid sequence (SEQ ID NO:894) of the human X-prolyl aminopeptidase (aminopeptidase P) 2 protein, XPNPEP2 (Genbank Accession No: NM_{—}003399.3) comprising, or alternatively consisting of, one or more of the predicted polynucleotide polymorphic loci, which include but are not limited to the following polynucleotide polymorphisms: XPNPEP2-G2172A (SNP_ID: AE116s18). The standard one-letter abbreviation for amino acids is used to illustrate the deduced amino acid sequence. The polynucleotide sequence contains a sequence of 3431 nucleotides (SEQ ID NO:893), encoding a polypeptide of 674 amino acids (SEQ ID NO:894). The polynucleotide polymorphic sites are represented by an “N”, in bold. The present invention encompasses the polynucleotide at nucleotide position 2172 as being either a “G” or an “A” of FIGS. 6A-D (SEQ ID NO:893), in addition to any combination thereof.

FIGS. 7A-C show the polynucleotide sequence (SEQ ID NO:895) and deduced amino acid sequence (SEQ ID NO:896) of the human guanylate cyclase 1, soluble, alpha 2 protein, GUCY1A2 (Genbank Accession No: NM_{—}000855.1) comprising, or alternatively consisting of, one or more of the predicted polynucleotide polymorphic loci which include but are not limited to the following polynucleotide polymorphisms: GUCY1A2-C2169T (SNP_ID: AE117s11), GUCY1A2-A825G (SNP_ID: AE117s7), and/or GUCY1A2-C822T (SNP_ID: AE117s6). The standard one-letter abbreviation for amino acids is used to illustrate the deduced amino acid sequence. The polynucleotide sequence contains a sequence of 2236 nucleotides (SEQ ID NO:895), encoding a polypeptide of 699 amino acids (SEQ ID NO:896). The polynucleotide polymorphic sites are represented by an “N”, in bold. The polypeptide polymorphic sites are represented by an “X”, in bold. The present invention encompasses the polynucleotide at nucleotide position 2169 as being either a “C” or a “T”, the polynucleotide at nucleotide position 825 as being either an “A” or a “G”, and/or the polynucleotide at nucleotide position 822 as being either a “C” or a “T” of FIGS. 7A-C (SEQ ID NO:895), in addition to any combination thereof.

FIG. 8 illustrates an example of the possible haplotypes (A, B, C, and D) for an individual that has the following genotype at a particular genomic locus: A/G heterozygote at SNP1, G/C heterozygote at SNP2, and A/C heterozygote at SNP3.

FIG. 9 illustrates an example of how the haplotype of an individual at a particular genomic locus can be determined using a combination of the individuals genotype with the genotypes of the individuals parents genotypes at the same locus. The example is based upon one parent having an A/A genotype at SNP1, a G/C genotype at SNP2, and an A/A genotype at SNP3, and the other parent having an A/G genotype at SNP1, C/C genotype at SNP2, and C/C genotype at SNP3, and the child being heterozygote at all three SNPs. As shown, there is only one possible haplotype combination. The later is based upon the absence of a crossing over event at this locus during meiosis.

FIGS. 10A-C illustrates an alignment of various metrin sequences with domain annotations, in addition to noting the location of the coding SNPs of the MEPB1 polypeptide of the present invention.

FIGS. 11A-E illustrates an alignment of various metrin sequences with domain annotations, in addition to noting the location of the coding SNPs of the ANPEP polypeptide of the present invention.

FIGS. 12A-B illustrates an alignment of various metrin sequences with domain annotations, in addition to noting the location of the coding SNPs of the ANPEP polypeptide of the present invention.

Table I provides a detailed summary of the reference alleles of the SNPs of the present invention (SEQ ID NO:1 to 147). ‘GENE_DESCRIPTION’ refers to the gene in which the SNP resides; ‘HGNC_ID’ refers to the gene symbol as designated by the HUGO Gene Nomenclature Committee; ‘SNP_ID’ refers to the unique internal name identifier associated with the SNP of the present invention; ‘REFSEQ_FLANK_REF’ refers to the genomic polynucleotide sequence of the gene immediately flanking the SNP of the present invention in both the 5′ and 3′ direction containing the reference allele; ‘REFSEQ_FLANK_REF SEQ ID NO:’ refers to the unique sequence number assigned to this sequence in the Sequence Listing; ‘REF_NT’ refers to the reference allele within the reference genomic nucleotide sequence for each respective polymorphic locus; ‘EXON’ refers to the location of each predicted SNP on the gene in which the SNP resides; ‘MUTATION_TYPE’ refers to the type of polymorphism for each SNP (e.g., “NON-CDS” for a SNP that resides outside the coding region, “Missense” for a SNP that resides within the coding region and results in a change in the amino acid sequence of the protein encoded by the SNP in which the SNP resides; and “Silent” for a SNP that resides within the coding region and does not result in a change in the amino acid sequence of the protein encoded by the SNP in which the SNP resides; ‘REVCOMP’ refers to the relative 5′ to 3′ orientation of the reference genomic polynucleotide sequence compared to the cDNA polynucleotide sequence of the gene wherein ‘0’ indicates the genomic and cDNA sequences are in the same orientation, whereas ‘1’ indicates the genomic and cDNA sequences are in an opposing orientation; ‘CDNA_SEQ_ID’ refers to the Genbank accession number of the wild type cDNA sequence of the gene in which the predicted SNP resides; and ‘CDNA_SEQ_POS’ refers to the nucleotide position of the predicted SNP on the cDNA sequence in which the SNP resides.

Table II provides a detailed summary of the alternate alleles for the SNPs of the present invention (SEQ ID NO: 148 to 294). ‘GENE_DESCRIPTION’ refers to the gene in which the SNP resides; ‘HGNC_ID’ refers to the gene symbol as designated by the HUGO Gene Nomenclature Committee; ‘SNP_ID’ refers to the unique internal name identifier associated with the SNP of the present invention; ‘REFSEQ_FLANK_ALT’ refers to the genomic polynucleotide sequence of the gene immediately flanking the SNP of the present invention in both the 5′ and 3′ direction containing the reference allele; ‘REFSEQ_FLANK_REF SEQ ID NO:’ refers to the unique sequence number assigned to this sequence in the Sequence Listing; ‘ALT_NT’ refers to the variant allele within the reference genomic nucleotide sequence for each respective polymorphic locus; ‘EXON’ refers to the location of each predicted SNP on the gene in which the SNP resides; ‘MUTATION_TYPE’ refers to the type of polymorphism for each SNP (e.g., “NON-CDS” for a SNP that resides outside the coding region, “Missense” for a SNP that resides within the coding region and results in a change in the amino acid sequence of the protein encoded by the SNP in which the SNP resides; and “Silent” for a SNP that resides within the coding region and does not result in a change in the amino acid sequence of the protein encoded by the SNP in which the SNP resides; ‘REVCOMP’ refers to the relative 5′ to 3′ orientation of the reference genomic polynucleotide sequence compared to the cDNA polynucleotide sequence of the gene wherein ‘0’ indicates the genomic and cDNA sequences are in the same orientation, whereas ‘1’ indicates the genomic and cDNA sequences are in an opposing orientation; ‘CDNA_SEQ_ID’ refers to the Genbank accession number of the wild type cDNA sequence of the gene in which the predicted SNP resides; and ‘CDNA_SEQ_POS’ refers to the nucleotide position of the predicted SNP on the cDNA sequence in which the SNP resides.

Table III provides a detailed summary of the alleles for the SNPs of the present invention including the reference and alternate codons and reference and alternate amino acid changes, as applicable (SEQ ID NO: 1 to 147; and 148 to 294). ‘GENE_DESCRIPTION’ refers to the gene in which the SNP resides; ‘HGNC_ID’ refers to the gene symbol as designated by the HUGO Gene Nomenclature Committee; ‘SNP_ID’ refers to the unique internal name identifier associated with the SNP of the present invention; ‘CONTIG_NUM’ refers to the name of the contig in which each SNP was detected; ‘CONTIG_POS’ refers to the nucleotide position on the contig in which the polymorphic locus of each SNP resides; ‘REF_SEQ_ID’ refers to the Genbank Accession No. of the genomic sequence in which the reference allele of the SNP resides; ‘REF_SEQ_POS’ refers to the nucleotide position of the polymorphic locus of each SNP; ‘REF_AA’ refers to the predicted reference amino acid sequence encoded by each reference SNP allele; ‘ALT_AA’ refers to the predicted alternate amino acid sequence encoded by each alternate SNP allele; ‘EXON’ refers to the location of each predicted SNP on the gene in which the SNP resides; ‘MUTATION_TYPE’ refers to the type of polymorphism for each SNP (e.g., “NON-CDS” for a SNP that resides outside the coding region, “Missense” for a SNP that resides within the coding region and results in a change in the amino acid sequence of the protein encoded by the SNP in which the SNP resides; and “Silent” for a SNP that resides within the coding region and does not result in a change in the amino acid sequence of the protein encoded by the SNP in which the SNP resides; ‘REVCOMP’ refers to the relative 5′ to 3′ orientation of the reference genomic polynucleotide sequence compared to the cDNA polynucleotide sequence of the gene wherein ‘0’ indicates the genomic and cDNA sequences are in the same orientation, whereas ‘1’ indicates the genomic and cDNA sequences are in an opposing orientation; ‘REF_CODON’ refers to the reference codon sequence in which the reference SNP allele resides; ‘ALT_CODON’ refers to the alternate codon sequence in which the alternate SNP allele resides; ‘PROT-EIN_ID’ refers to the Genbank accession number of the wild type protein sequence encoded by the gene in which the predicted SNP resides; and ‘PROT-EIN_POS’ refers to the amino acid position of the variant amino acid allele encoded by the gene in which the predicted encoding SNP resides.

Table IV provides a detailed summary of the various primers that were used to amplify the sequence surrounding each SNP of the present invention. The Table headings are the same as in Table I, II, and III above with the following exceptions: ‘PCR_AMPLICON_NAME’ refers to the name of the PCR product produced from PCR amplification using the described PCR_LEFT_PRIMER and PCR_RIGHT_PRIMER; ‘PCR_LEFT_PRIMER’ refers to the sequence of the left (upstream) PCR primer used to amplify across the polymorphic locus of each predicted SNP; ‘PCR_LEFT_PRIMER (SEQ ID NO:)’ refers to the unique sequence number assigned to this sequence in the Sequence Listing; ‘PCR_RIGHT_PRIMER’ refers to the sequence of the right (downstream) PCR primer used to amplify across the polymorphic locus of each predicted SNP; ‘PCR_RIGHT_PRIMER (SEQ ID NO:)’ refers to the unique sequence number assigned to this sequence in the Sequence Listing.

Table V provides a detailed summary of the various primers that were used to sequence the amplified PCR product of the primers listed in Table W. The sequence of each PCR amplicon was used in the identification of the SNPs of the present invention. The Table headings are the same as in Table I, II, and III above with the following exceptions: ‘FORWARD_SEQUENCING_PRIMER’ refers to the sequence of the left (upstream) primer used to sequencing across the PCR amplicon of each predicted SNP; ‘FORWARD_SEQUENCING_PRIMER’ (SEQ ID NO:)′ refers to the unique sequence number assigned to this sequence in the Sequence Listing; ‘REVERSE_SEQUENCING_PRIMER’ refers to the sequence of the left (downstream) primer used to sequencing across the PCR amplicon of each predicted SNP;; ‘REVERSE_SEQUENCING_PRIMER’ (SEQ ID NO:)′ refers to the unique sequence number assigned to this sequence in the Sequence Listing.

Table VI provides a detailed summary of the various primers that were used in genotyping the single nucleotide polymorphisms of the angioedema candidate genes of the present invention for identifying their putative association to the angioedema phenotype. The Table headings are the same as in Table I, II, and III above with the following exceptions: ‘ORCHID_FORWARD’ refers to the 3′ (forward) primer used for sequencing across the SNP loci of each respective SNP; ‘ORCHID_FORWARD’ (SEQ ID NO:)′ refers to the SEQ ID NO for this particular sequence within the Sequence Listing of the present invention; ‘ORCHID_REVERSE’ refers to the 5′ (reverse) primer used for sequencing across the SNP loci of each respective SNP; and ‘ORCHID_REVERSE’ (SEQ ID NO:)′ refers to the SEQ ID NO for this particular sequence within the Sequence Listing of the present invention.

Table VII provides a detailed summary of the various primers that may be used in genotyping the single nucleotide polymorphisms of the angioedema candidate genes of the present invention for identifying their putative association to the angioedema phenotype. The Table headings are the same as in Table I, II, and III above with the following exceptions:; ‘GBS_LEFT’ refers to the 3′ (forward) primer that may be used for sequencing across the SNP loci of each respective SNP; ‘GBS_LEFT (SEQ ID NO:)’ refers to the SEQ ID NO for this particular sequence within the Sequence Listing of the present invention; ‘GBS_RIGHT’ refers to the 5′ (reverse) primer that may be used for sequencing across the SNP loci of each respective SNP; and ‘GBS_RIGHT (SEQ ID NO:)’ refers to the SEQ ID NO for this particular sequence within the Sequence Listing of the present invention.

Table VIII provides a summary of the various DNA samples, in addition to their ethnic origin and disease phenotype, used in identifying the single nucleotide polymorphisms of the angioedema candidate genes of the present invention.

### DETAILED DESCRIPTION OF THE INVENTION

The present invention relates to a nucleic acid molecule which comprises a single nucleotide polymorphism (SNP) at a specific location. The nucleic acid molecule, e.g., a gene, which includes the SNP has at least two alleles, referred to herein as the reference allele and the variant allele. The reference allele (prototypical or wild type allele) has been designated arbitrarily and typically corresponds to the nucleotide sequence of the native form of the nucleic acid molecule. The variant allele differs from the reference allele by one nucleotide at the site(s) identified in the Table I, III, and/or IV. The present invention also relates to variant alleles of the described genes and to complements of the variant alleles. The invention further relates to portions of the variant alleles and portions of complements of the variant alleles which comprise (encompass) the site of the SNP and are at least nucleotides in length. Portions can be, for example, 5-10,5-15, .10-20,5-25, 10-30, 10- or 10-100 bases long. F or example, a portion of a variant allele which is nucleotides in length includes the single nucleotide polymorphism (the nucleotide which differs from the reference allele at that site) and twenty additional nucleotides which flank the site in the variant allele. These additional nucleotides can be on one or both sides of the polymorphism. Polymorphisms which are the subject of this invention are defined in Table I, III, and/or IV herein.

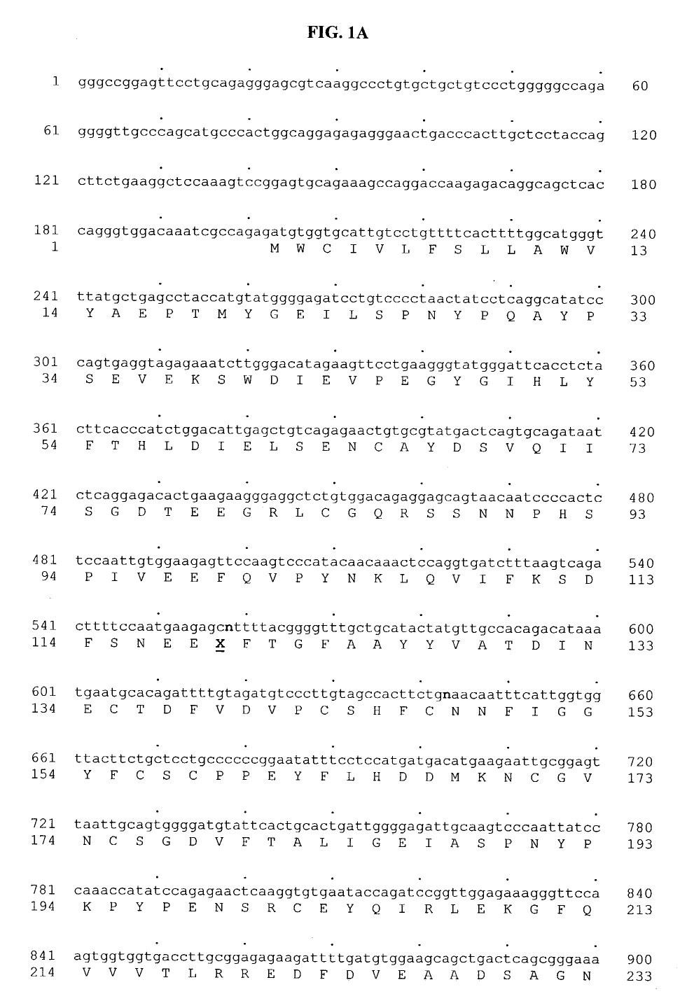

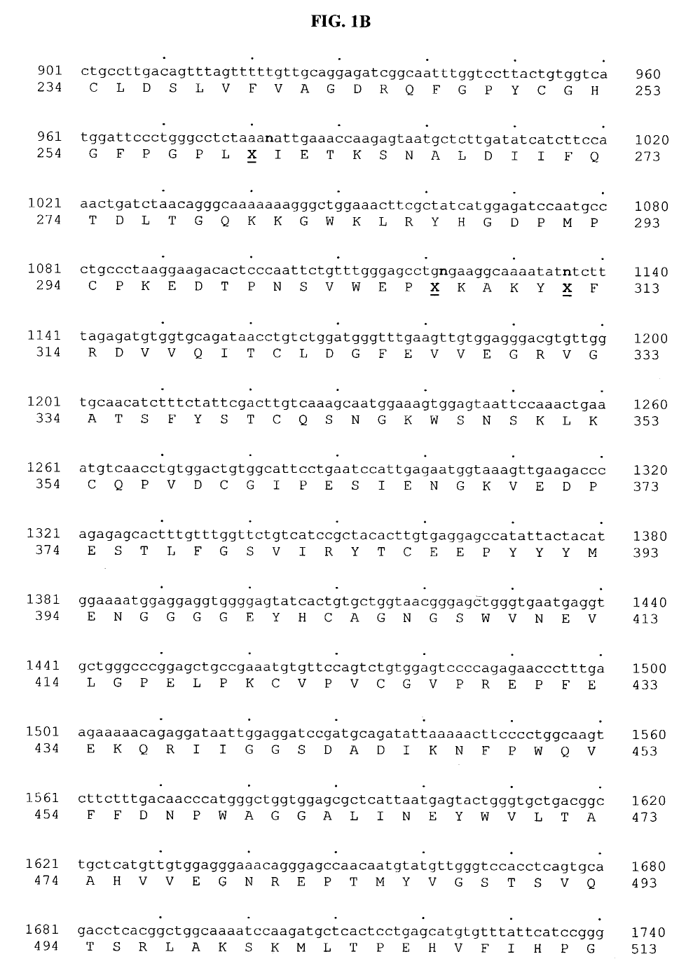

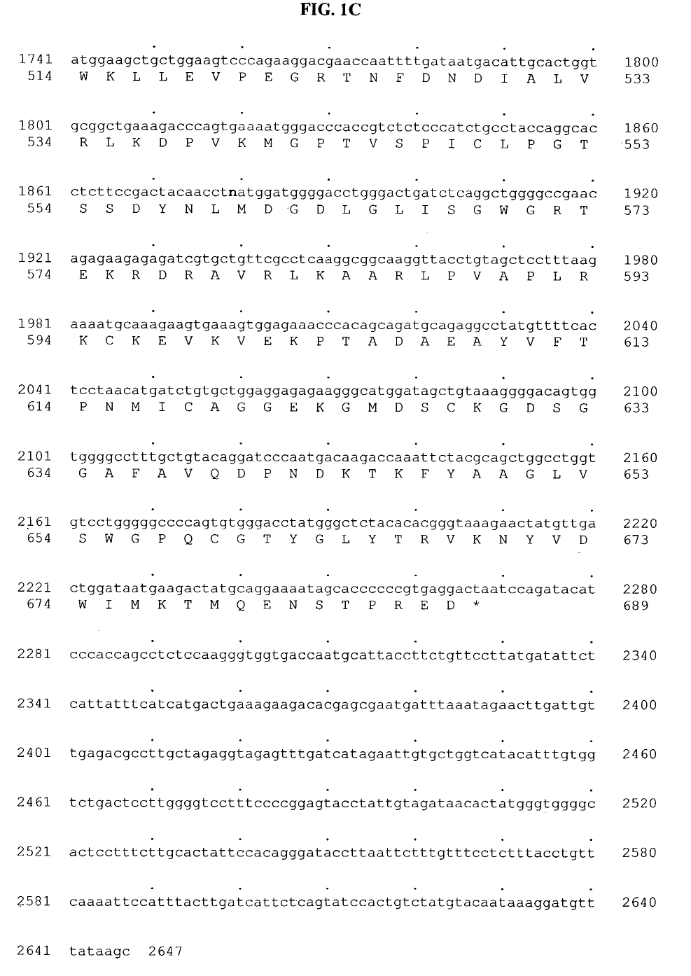

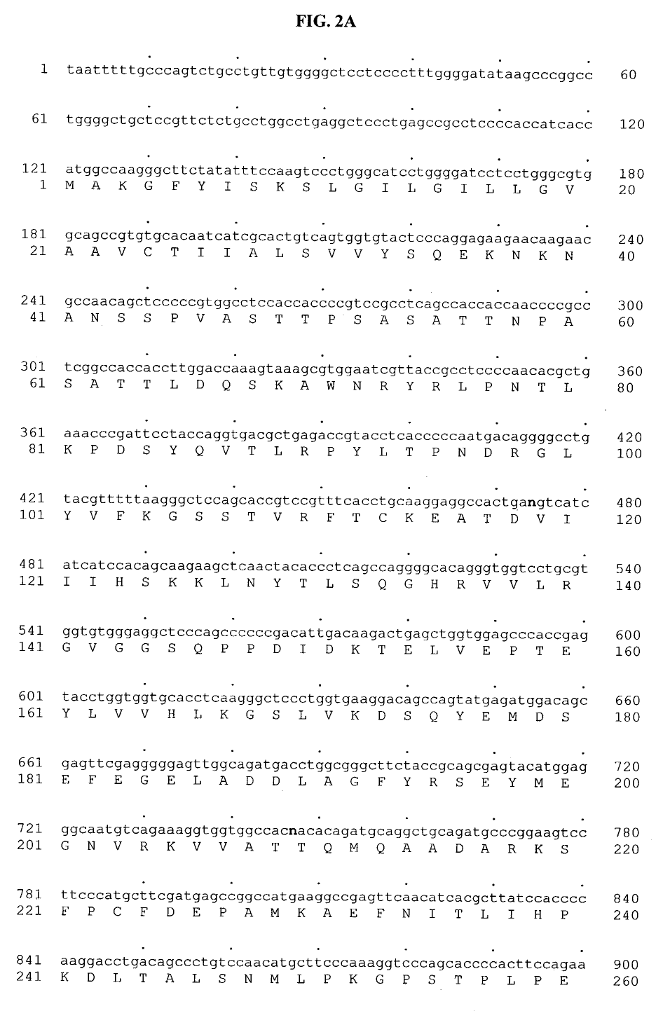

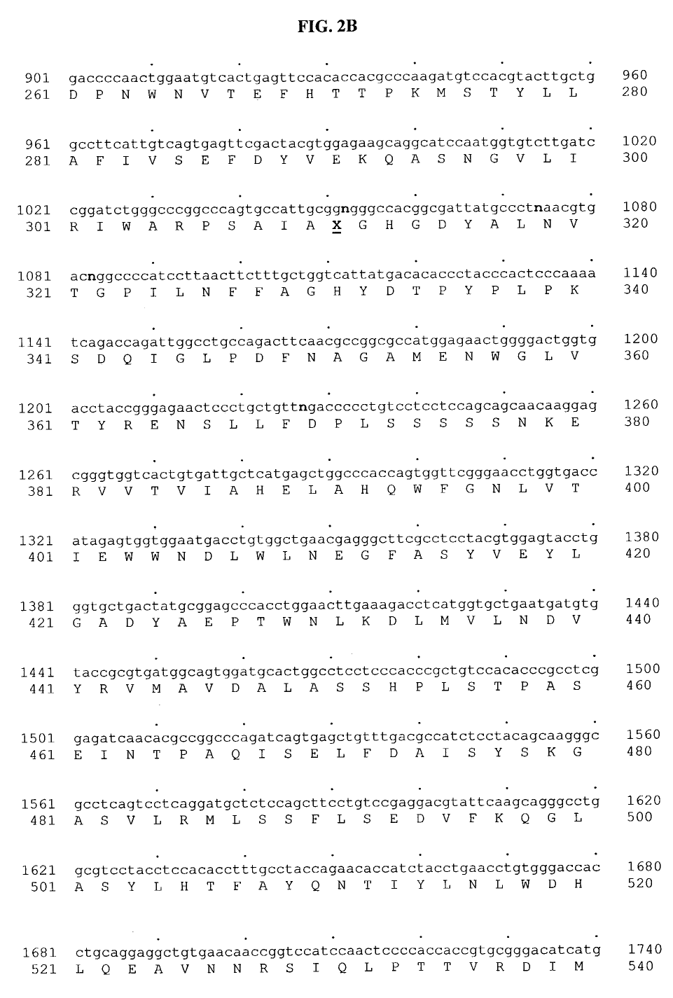

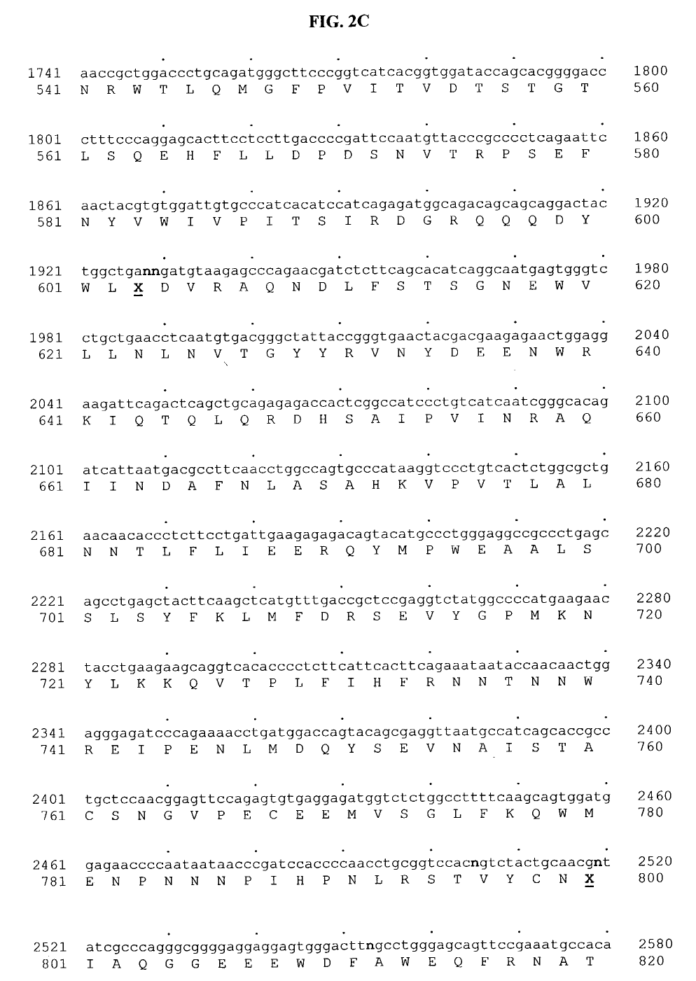

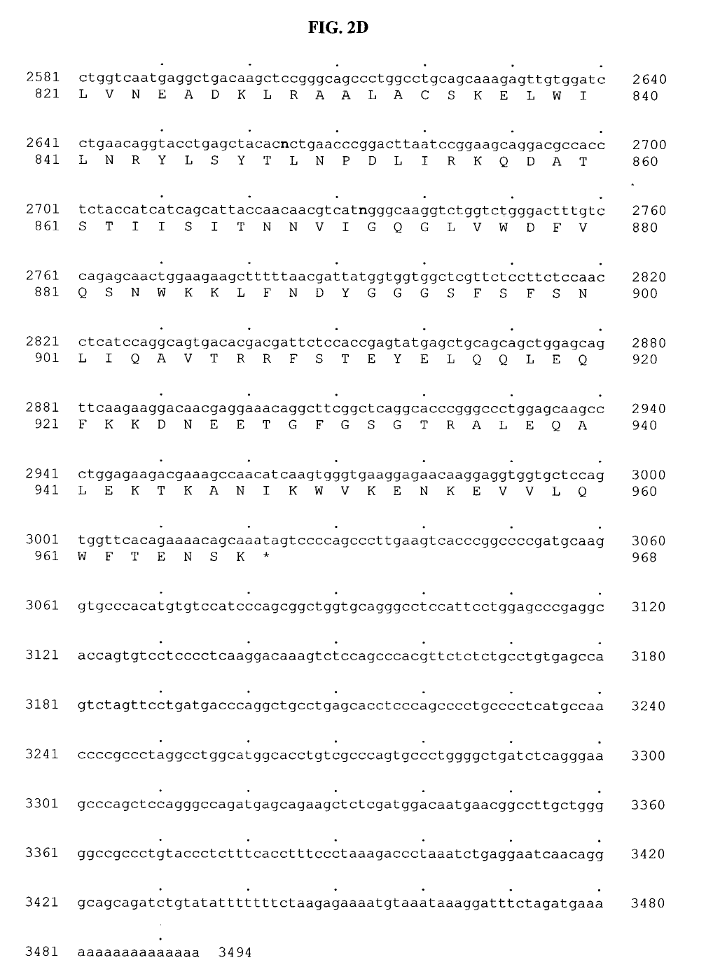

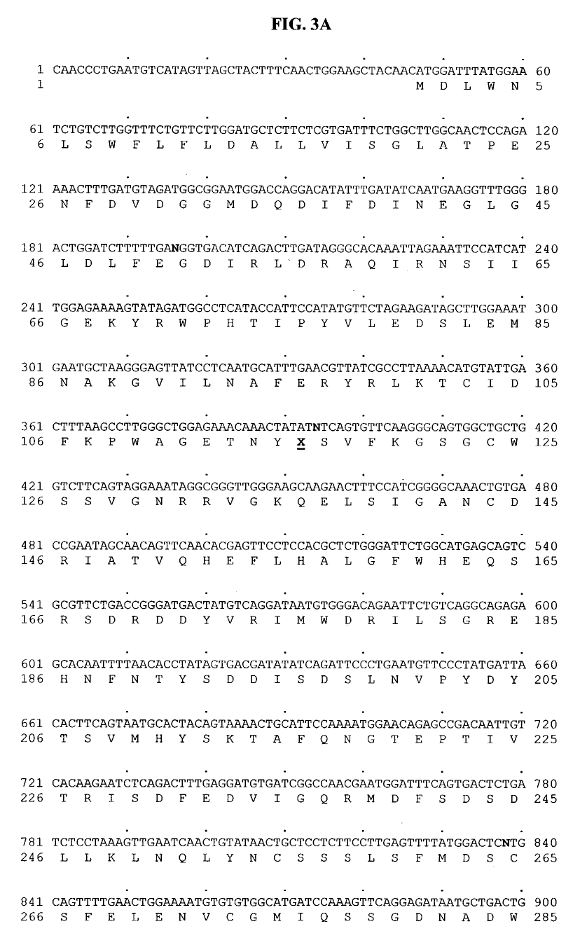

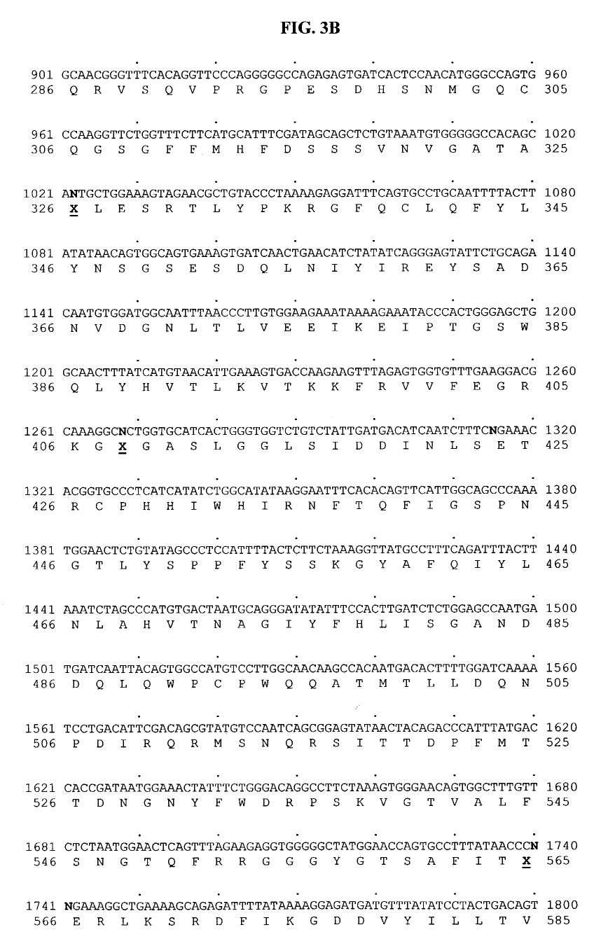

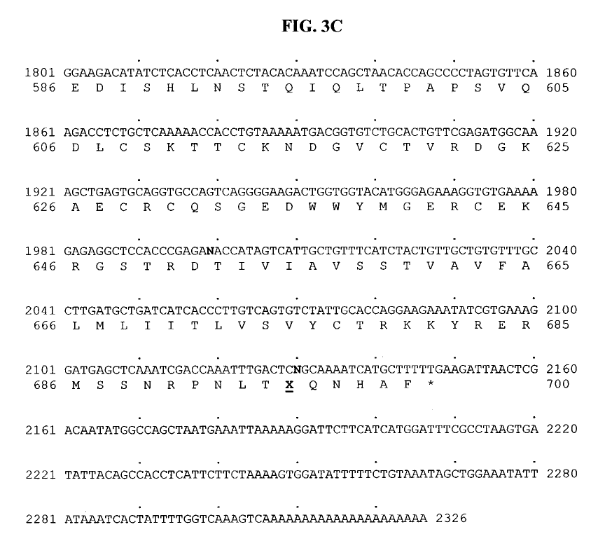

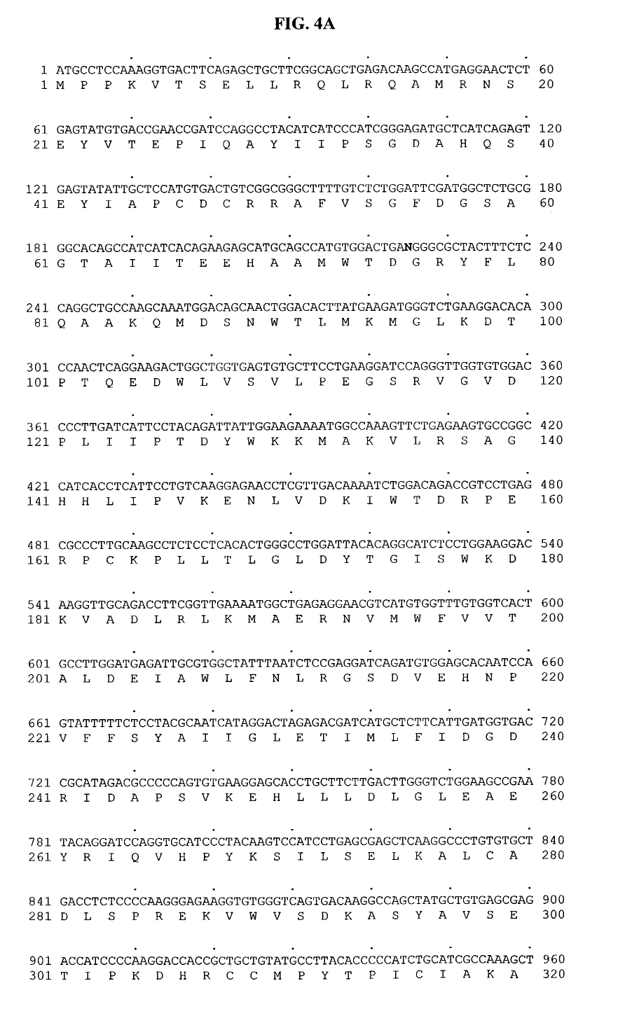

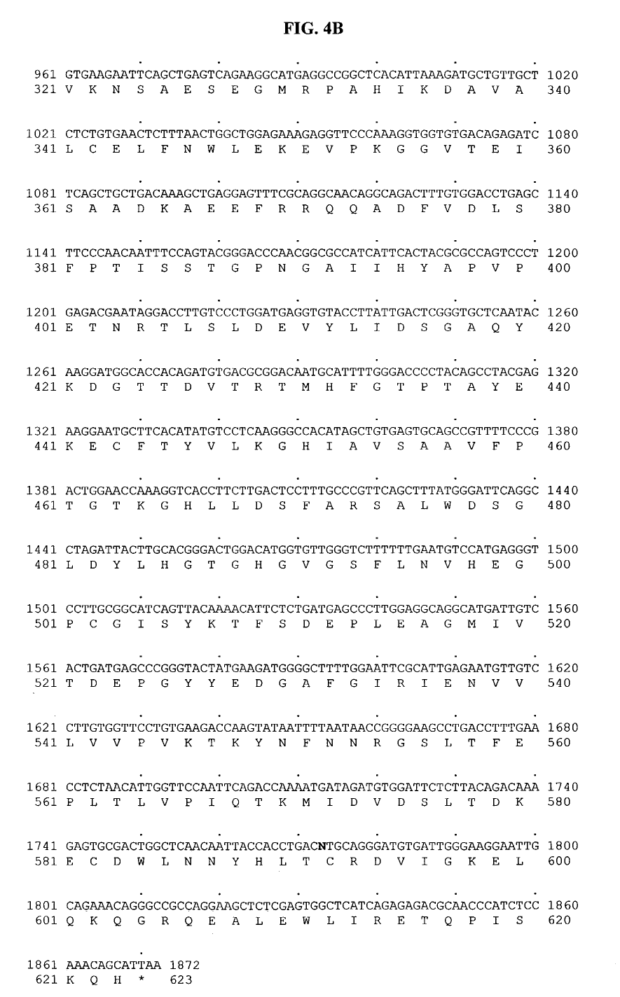

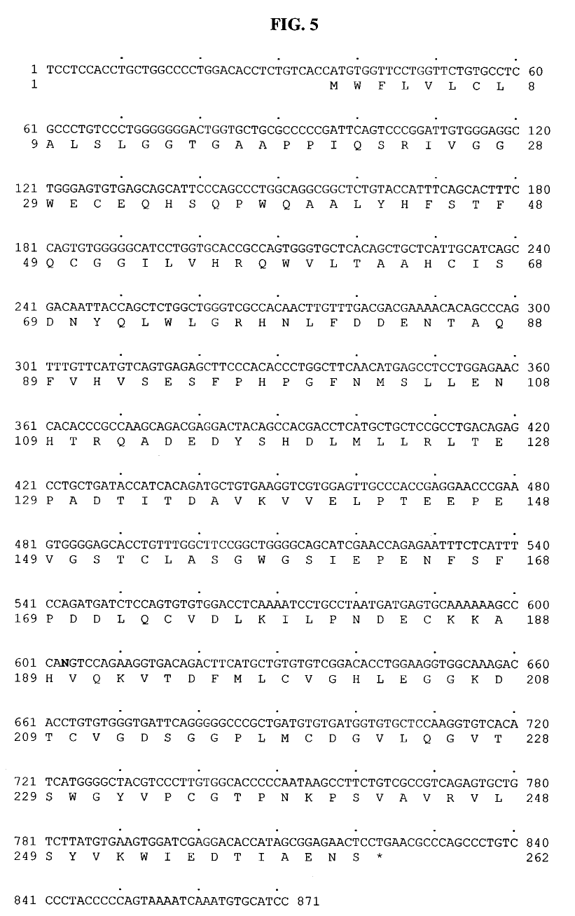

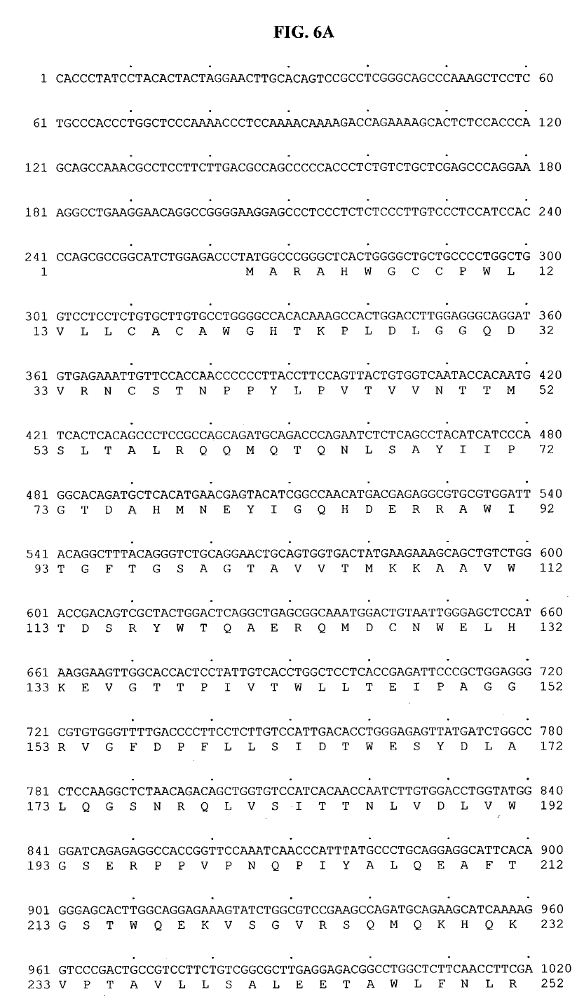

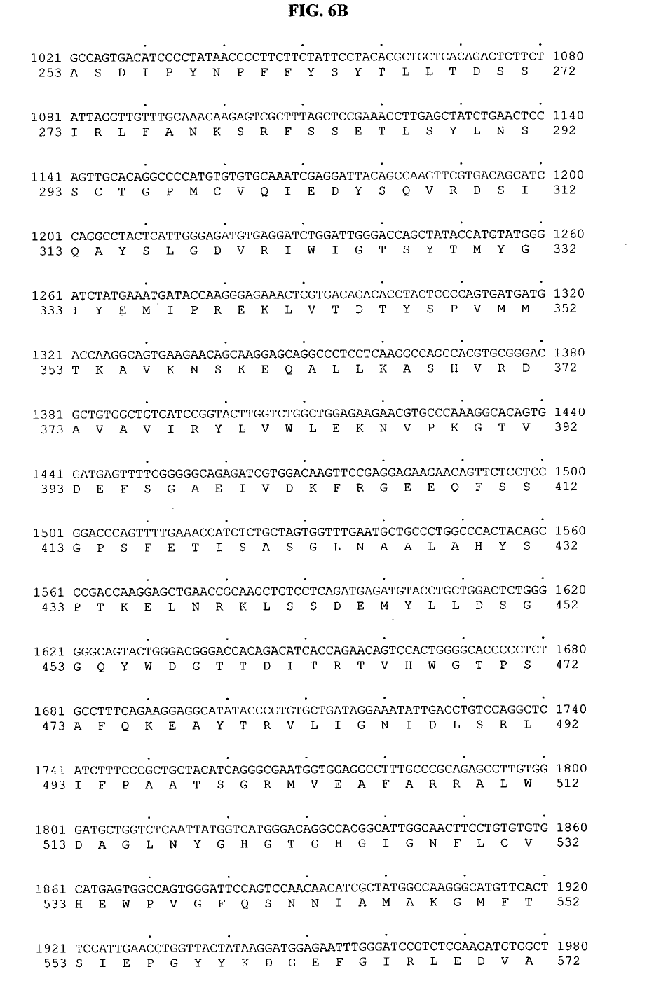

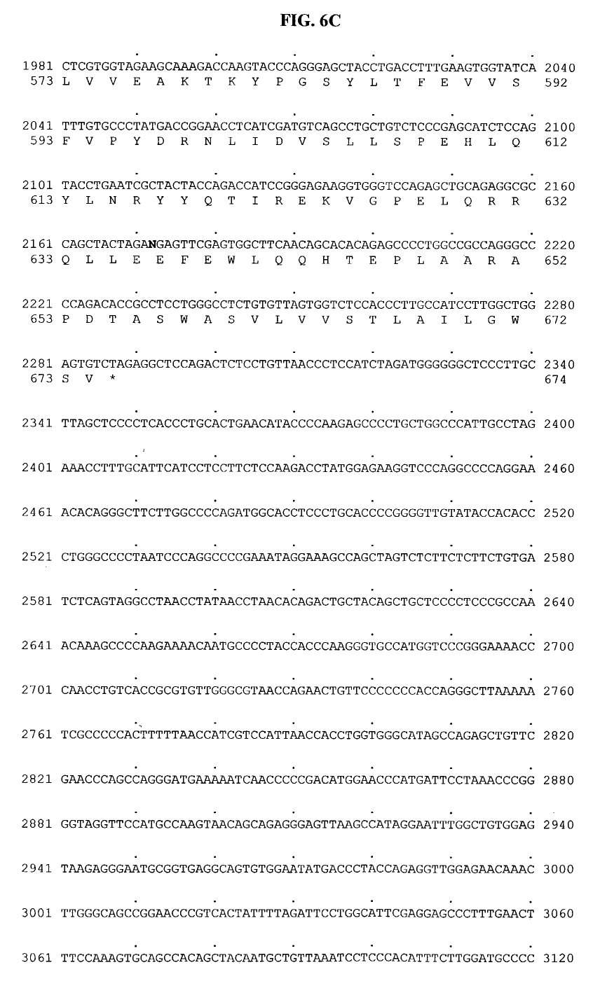

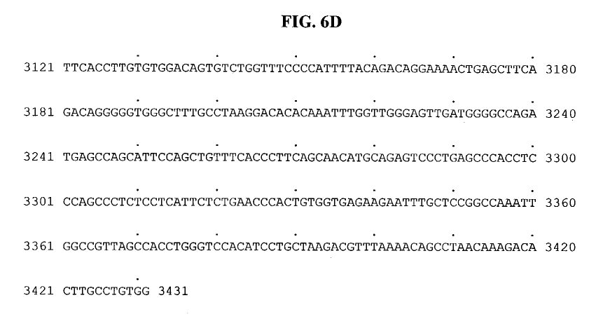

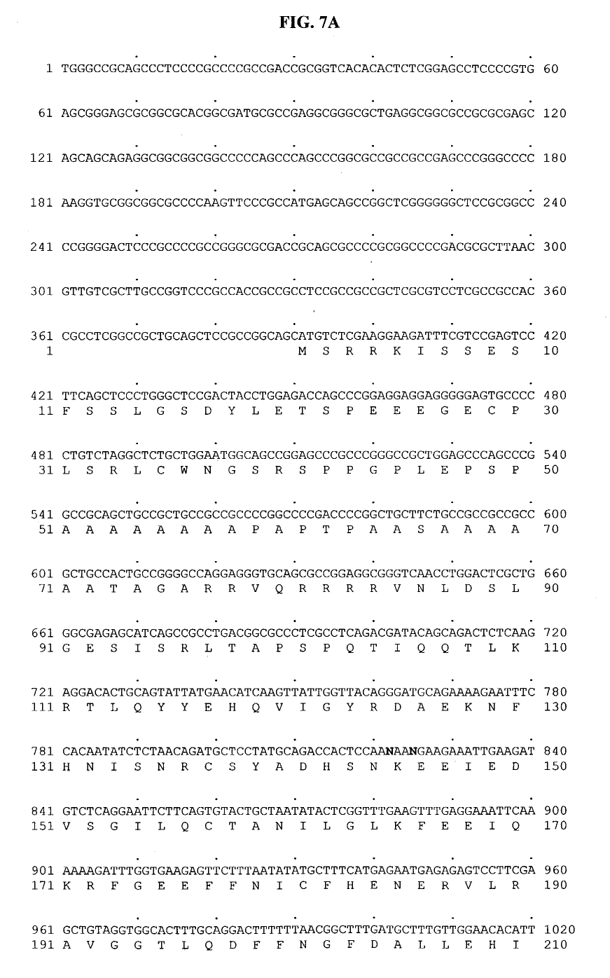

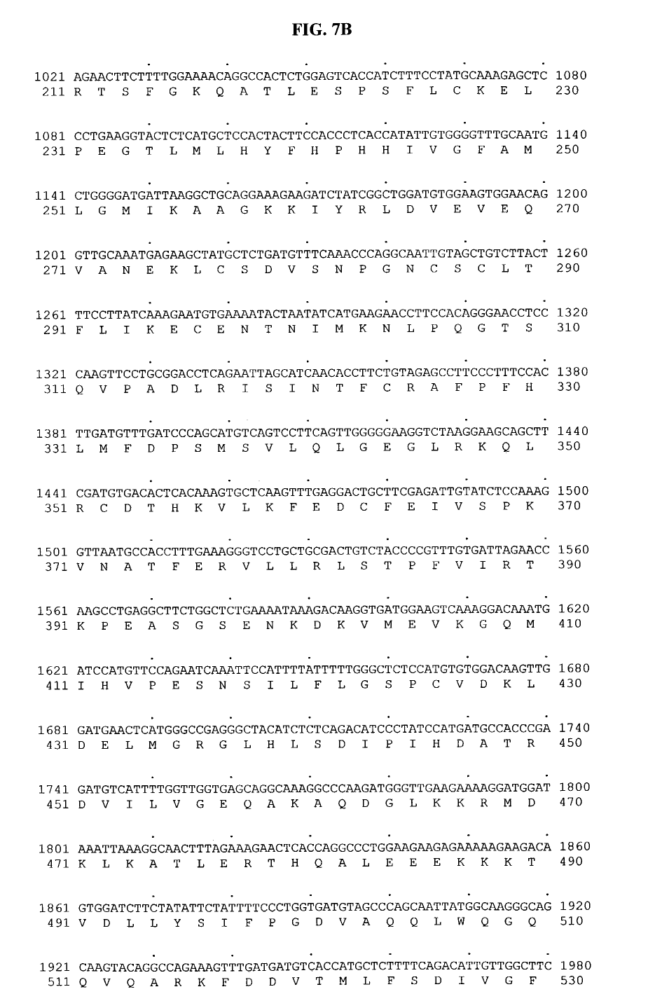

For example, the invention relates to a portion of a gene (e.g., human complement component 1, s subcomponent protein (C1S) having a nucleotide sequence according to FIGS. 1A-C (SEQ ID NO:883) comprising a single nucleotide polymorphism at a specific position (e.g., nucleotide 558). The reference nucleotide for this polymorphic form of C1S is shown in the ‘FLANK_SEQ (REF/ALT)’ column as the “REF” nucleotide (in this case, the “REF” nucleotide is “G”) of Table I, and the variant nucleotide is shown in the ‘FLANK_SEQ (REF/ALT)’ column as the “ALT” nucleotide of Table I (in this case, the “ALT” nucleotide is an “A”). In a preferred embodiment, the nucleic acid molecule of the invention comprises the variant (alternate) nucleotide at the polymorphic position. For example, the invention relates to a nucleic acid molecule which comprises the nucleic acid sequence shown in the ‘FLANK_SEQ (REF/ALT)’ as the “ALT” nucleotide in Table I having an “A” at nucleotide position 558 of FIGS. 1A-C (SEQ ID NO:883). The nucleotide sequences of the invention can be double- or single-stranded.

The single nucleotide polymorphisms described herein derive from genes that the inventors of the present invention believe are associated with the incidence of angioedema or angioedema-like events. Specifically, the single nucleotide polymorphisms of any one or more of the genes described herein may increase an individuals susceptibility to acquiring angioedema or an angioedema-like event, especially upon the administration of an ACE, or vasopeptidase, inhibitor among others.

The genes that are thought to harbor single nucleotide polymorphisms associated with angioedema were carefully chosen by the inventors of the present invention based upon a variety of criterions and are provided, in brief, below.

The bradykinin pathway, and genes associated with the bradykinin pathway, are suspected as playing a role in the incidence of angioedema for several reasons: 1) bradykinin is a substrate of ACE, and thus expected to be increased in the presence of ACE inhibitors; 2) bradykinin causes microvascular leakage, which might be involved in the angioedema phenotype; 3) deficiencies in the blood coagulation pathway, such as a defect in Cl esterase inhibitor, is expected to alter the bradykinin level; and 4) bradykinin levels are suspected to be increased during acute drug induced angioedema and hereditary angioedema (Nussberger, Cugno et al. 1998; Nussberger J 1999).

Members of the bradykinin pathway include, for example, the aminopeptidase P protein (XPNPEP2), the bradykinin B1 receptor (BDKRB1), the bradykinin B2 receptor (BDKRB2), the NK1 tachykinin receptor (TACR1), the C1 esterase inhibitor protein (C1NH), the tissue kallikrein protein (KLK1), angiotension converting enzyme 2 (ACE2), and the kallistatin protein (PI4; also referred to as SERPINA4). The bradykinin B1 receptor, the bradykinin B2 receptor, and the NK1 tachykinin receptor are involved in bradykinin signal transduction, while the other five proteins affect the production/degradation of bradykinin and other active kinins. These proteins, in addition to others described herein, have been selected for analysis of potential single nucleotide polymorphims in their encoding polynucleotide sequence based upon their participation in the bradykinin pathway.

Aminopeptidase P is a hydrolase that is specific for N-terminal imido bonds. Structurally, the enzyme is a member of the ‘pita bread fold’ family and occurs in mammalian tissues in both soluble and GPI-anchored membrane-bound forms. The deduced XPNPEP2 protein has 673 amino acids and an estimated molecular mass of 75,490 Da. The human and pig XPNPEP2 amino acid sequences show significant evolutionary divergence, with 83% identity; 5 of 6 potential N-glycosylation sites, and 5 of 6 cysteine residues that are potentially involved in disulfide bond formation, are conserved.

The bradykinin B1 and B2 receptors are G-protein coupled receptors with seven trans-membrane domains. The bradykinin B1 receptor is bradykinin inducible, while the bradykinin B2 receptor is constitutively expressed.

The NK1 tachykinin receptor is a receptor for tachykinins, which include, for example, substance P. The NK1 tachykinin receptor is a G-protein coupled receptor with seven trans-membrane domains. Bradykinin binding to the bradykinin B2 receptor causes the release of neuropeptides, such as substance P, ultimately leading to the activation of the NK1 tachykinin receptor.

C1 esterase inhibitor regulates the first component of complement (C1) by inhibition of the proteolytic activity of its subcomponents C1r and C1s. Such inhibition prevents activation of C4 and C2 by C1s. C1I also inhibits several other serine proteinases including plasmin, kallikrein, and coagulation factors XIa and XIIa. (Davis, A. E., III et al., Proc. Nat. Acad. Sci. 83: 3161-3165, 1986.) The C1 esterase inhibitor is known to comprise a 22-residue signal peptide at the N-terminal end of the protein.

Tissue kallikrein is a serine protease that is involved in the post-translational processing of peptides. The post-translational processing activity of the tissue kallikrein protein includes the generation of bradykinin from high molecular weight kininogen.

ACE2 is a zinc metalloprotease that shares significant sequence homology to angiotensin converting enzyme (ACE, DCP1), and like ACE, ACE2 cleaves angiotensin I in vitro. Moreover, it has been shown that des-Arg bradykinin is also a substrate for both ACE and ACE2 in vitro. des-Arg bradykinin is an active derivative of bradykinin, and it has been suggested that an increased level of this molecule may cause angioedema (Blais, C. et al., Immunopharmacology 1999;43:293-302). ACE inhibition by ACE inhibitors, which include the vasopeptidase inhibitor Omapatrilat, is expected to increase the local concentration of des-Arg bradykinin—such an effect may be the mechanism of ACE inhibitor and/or Omapatrilat-induced angioedema. Assuming the latter model is correct, the expression level of other proteases that can inactivate des-Arg bradykinin, such as ACE2, may determine one's susceptibility to angioedema upon ACE inhibitor (or Omapatrilat) treatment. For example, individuals with low ACE2 activity may be more sensitive to angioedema due to these individuals inability to rapidly degrade des-Arg bradykinin when ACE is inhibited. ACE2 has been shown to be insensitive to ACE inhibitors. The ACE2 protein is known to comprise the following features: a HEXXH motif (His374-His378); a Zn-binding motif, Glu406; Zn-binding, Ser740-Phe762; and a transmembrane domain (Tipnis, S. et al, (2000) J. Biol. Chem . . . 275, 33238-33243).

Kallistatin (PI4) tightly binds and inhibits tissue kallikrein, which is a key protease for generation of bradykinin and other kinins. Bradykinin and another active kinin, kallidin, are generated by cleavage of kininogens by kallikreins, which include tissue kallikrein. Thus, protein and activity levels of kallistatin can have a direct effect on the amount of bradykinin and other kinin levels. Since these kinin molecules are potentially involved in the angioedema phenotype, a molecule which can affect the kinin level, such as kallistatin, may also have an involvement in angioedema. Kallistatin is also shown to be a potent vasodilator. The kallistatin protein is a new member of the serpin superfamily and represents a major inhibitor of human tissue kallikrein in the circulation. Amino acid residues Lys386 (P3), Phe387 (P2), Phe388 (P1), Ser389 (P1′), and Ala390 (P2′) are involved in the binding to the active site of tissue kallikrein and its inhibition. The translated amino acid sequence of kallistatin matches the protein sequence and shares 44 to 46% sequence identity with human alpha-1-antichymotrypsin (AACT; 107280), alpha-1-antitrypsin (PI; 107400), corticosteroid-binding globulin (CBG; 122500), thyroxine-binding globulin of serum (TBG; 314200), and protein C inhibitor (PCI; 227300).

Proteins involved in the generation of bradykinin (BK) and BK-related peptides (referred to as kinins) also include the following: Plasma kallikrein (KLKB1), a2-macroglobulin (A2M), alpha-1 antiproteinase, antitrypsin (SERPINA1), and Kininogen (KNG). Kininogen (KNG) gene encodes precursors (HMW kininogen and LMW kininogen) for bradykinin (BK) and other BK-related kinin peptides. Kallikreins (KLK1, KLK2, and KLKB1) are serine proteases that cleave kininogens and liberate kinin (Campbell, D.,. Braz J Med Biol Res 33, 665-677 (2000); and Blais, C. J. et al.,. Peptides 21, 1903-1940 (2000)). Alpha2-macrogulobulin (A2M), protease inhibitor 4 (PI4), and alpha-1 antiprotease (SERPINA1) are serine protease inhibitors that are known to inhibit kallikreins. Therefore, the inventors of the present invention have conceived that variants of the KLKB1, A2M, SERPINA1, KNG, KLK1, KLK2, KLKB1, A2M, P14, and SERPINA1 are associated with the incidence of angioedema or angioedema-like events, in general, or in conjunction with the administration of an ACE or vasopeptidase inhibitor.

Single nucleotide polymorphisms in genes that either belong to or have an affect on the complement pathway were also investigated relative to the increasing an individuals susceptibility to angioedema or angioedema-like events. Such complement pathway genes include the following: C1, Q subcomponent alpha (C1QA), C1, Q subcomponent beta (C1QB), C1, S subcomponent (C1S), C1 esterase inhibitor (C1NH), Factor XII (F12), and Tissue plasminogen (PLG).

Defects in the C1NH gene are known to cause a hereditary form of angioedema⁴. Therefore, the inventors of the present invention have conceived that variations in the C1NH gene may affect the susceptibility of individual to angioedema in general, including the angioedema events induced by an ACE inhibitor or vasopeptidase inhibitor.

C1QA, C1QB and C1S are protease components in the complement pathway, which is inhibited by C1NH ((Ebo, D. et al., Acta Clin Belg 55, 22-29 (2000)). The PLG gene encodes a precursor for plasmin which is also known to be involved in activation of the complement pathway and is also inhibited by C1NH. FXII is a component of the blood coagulation pathway. FXII is involved both in the activation of the complement system and in complement generation. Therefore, the inventors of the present invention have conceived that variants of any one or more of the C1QA, C1QB, C1S , C1NH , F12, and/or PLG genes may alter the action of C1NH, resulting in one or more of these genes as being associated with the incidence of angioedema or angioedema-like events, in general, or in conjunction with the administration of an ACE or vasopeptidase inhibitor.

Single nucleotide polymorphisms in genes that are involved in bradykinin (BK) degradation were also investigated relative to increasing an individuals susceptibility to angioedema or angioedema-like events. Such bradykinin (BK) degradation genes include the following: angiotensin converting enzyme 2 (ACE2), alanyl aminopeptidase (ANPEP), chymase 1, mast cell (CMA1), carboxypeptidase U (CPB2), carboxypeptidase M (CPM), carboxypeptidase Ni (CPN1), angiotensin converting enzyme (DCP1), glutamyl aminopeptidase (ENPEP), leucyl/cystinal aminopeptidase (LNPEP), meprin A, alpha (MEP1A), meprin A, beta (MEP1B), neutral endopeptidase NEP 24.11 (MME), prolylcarboxypeptidase (PRCP), endopeptidase 24.15 (THOP1), aminopeptidase P (membrane bound) (XPNPEP2), and aminopeptidase P-like (XPNPEPL).

DCP1 and MME are known to be involved in bradykinin degradation. These two genes encode direct molecular targets of omapatrilat (vasopeptidase inhibitor) action, and it has been shown that inhibition of these enzymes with omapatrilat leads to a higher level of bradykinin (Blais,C. J. et al.,. J Pharmacol Exp Ther 295, 621-626 (2000)). ACE2 represents a recently discovered homologue of DCP1, which may have a potential involvement in bradykinin degradation. Kininase I group of proteases (CPB2, CPM, CPN1), THOP1 and XPNPEP2 have also been shown to be involved in the degradation of BK and BK-related kinins (Blais, C. J., et al., Peptides 21, 1903-1940 (2000)). XPNPEPL has a similar substrate specificity as XPNPEP2 and is also potentially capable of degrading BK and BK.-Other peptidases (ANPEP, CMA1, ENPEP, LNPEP, MEP1A, MEP1B, and PRCP) all have potential to be involved in BK and BK-related peptides, based on their substrate specificity. Therefore, the inventors of the present invention have conceived that variants of any one or more of the ACE2, ANPEP, CMA1, CPB2, CPM, CPN1, DCP1, ENPEP, LNPEP, MEP1A, MEP1B, MME, PRCP, THOP1, XPNPEP2, and/or XPNPEPL genes may be associated with the incidence of angioedema or angioedema-like events, in general, or in conjunction with the administration of an ACE or vasopeptidase inhibitor.

Single nucleotide polymorphisms in genes that are involved in BK and BK-related kinin signal transduction were also investigated relative to increasing an individuals susceptibility to angioedema or angioedema-like events. Such BK and BK-related kinin signal transduction genes include the following: bradykinin Bi receptor (BDKRB1), bradykinin B2 receptor (BDKRB2), nitric oxide synthase 2A (NOS2A), eNOS (NOS3), soluble guanylate cyclase 1, alpha-2 subunit (GUCY1A2), soluble guanylate cyclase 1, alpha-3 subunit (GUCY1A3), soluble guanylate cyclase 1, beta-2 subunit (GUCY1B2), soluble guanylate cyclase 1, beta-3 subunit (GUCY1B3), NK1 tachykinin receptor (TACR1), NK2 tachykinin receptor (TACR2), NK3 tachykinin receptor (TACR3), AKT kinase 2 (AKT2), and AKT kinase 3 (AKT3).

BDKRB1 and BDKRB2 both encode G-protein coupled receptor for BK and BK-related kinins (Marceau, F., et al., Clin Rev Allergy Immunol 16, 385-401 (1998)). Binding of BK and BK-related kinins is the first step of signal transduction by BDKRB1 and BDKRB2.

NOS2A and NOS3 encode nitric oxide (NO) synthases, and bradykinin is known to activate NO synthases (Marietta, M. et al., Trends Biochem Sci 26, 519-521 (2001)). Thus these molecules can be considered as part of signal transduction downstream of bradykinin. The inventors of the present invention have conceived that hyperactivation of these enzymes through genetic variation may enhance the observed angioedemphenotype or symptoms causing an individual to be more susceptible.

GUCY1A2, GUCY1A3, GUCY1B2 and GUCY1B3 all encode a subunit of soluble guanylate cyclase. Soluble guanylate cyclases are activated by NO and catalyze the formation of cGMP, which is a secondary messenger for signal transduction. At least part of the bradykinin action is mediated through these molecules, as bradykinin activates NO synthases. . The inventors of the present invention have conceived that variation in these genes may cause an enhanced bradykinin signal transduction.

Bradykinin also causes a release of substance P, which belongs to a tachykinin family of peptides. Thus substance P pathway can be considered to lie downstream of the bradykinin pathway. Substance P is also a substrate for angiotensin converting enzyme (DCP1), which is a target for vasopeptidase and ACE inhibitors. The inventors of the present invention have conceived that a genetic variation in substance P pathway has potential to contribute to vasopeptidase inhibitor induced, or ACE inhibitor induced angioedema phenotypes. TACR1 encodes a G-protein coupled receptor for substance P. TACR2 and TACR3 encode related G-protein coupled receptors for other related tachykinins (neurokinin A and neurokinin B). The relatedness of these molecule to TACR1 suggest some of the bradykinin signals may also be transduced through these molecules.

AKT kinases (AKT2, AKT3) induce signal transduction by NO synthases through phosphorylation (Marletta, M. et al., Trends Biochem Sci 26, 519-521 (2001)). As NO synthases are activated by bradykinin, AKT kinases can be considered to be in the same pathway as bradykinin signal transduction.

Therefore, the inventors of the present invention have conceived that variants of any one or more of the BDKRB1, BDKRB2, NOS2A, NOS3, GUCY1A2, GUCY1A3, GUCY1B2, GUCY1B3, TACR1, TACR2, TACR3, AKT2, and/or AKT3 genes may be associated with the incidence of angioedema or angioedema-like events, in general, or in conjunction with the administration of an ACE or vasopeptidase inhibitor. Moreover, the inventors of the present invention have conceived that variations in these genes may affect the intensity of downstream bradykinin signal transduction upon stimulation by BK or BK-related kinins. Such variants may cause the signal to be stronger, or can cause prolonged signal transduction due to decreased down regulation or desensitization. Therefore, such a phenomenon may contribute to the susceptibility of an individual to vasopeptidase inhibitor or ACE inhibitor induced angioedema by enhancing the effect of BK and BK-related kinins.

Single nucleotide polymorphisms in genes that are involved in angiotensin II signal transduction were also investigated relative to increasing an individuals susceptibility to angioedema or angioedema-like events. Such angiotensin II signal transduction genes include the following: angiotensin II receptor 1 (AGTR1), and angiotensin II receptor 2 (AGTR2).

AGTR1 and AGTR2 encode two of the G-protein coupled receptors for angiotensin II. The rate limiting step for angiotensin II generation is the conversion of angiotensin I to angiotensin II mediated by angiotensin converting enzyme (DPC1) (Weber, M., Am J Hypertens 12, 139S-147S (1999)). ACE inhibitor class of drugs and vasopeptidase inhibitors including Omapatrilat cause blood pressure reduction by inhibiting this conversion by DPC1, reducing angiotensin II generation. Thus, variant forms of AGTR1 and AGTR2 with an altered expression level, or a different affinity for angiotensin II may cause a hypersensitivity to the action of these drugs, leading to adverse side effects such as angioedema. Therefore, the inventors of the present invention have conceived that variants of any one or more of the AGTR1, and/or AGTR2 genes may be associated with the incidence of angioedema or angioedema-like events, in general, or in conjunction with the administration of an ACE or vasopeptidase inhibitor.

Single nucleotide polymorphisms in genes that are involved in atrial natriuretic factor signal transduction were also investigated relative to increasing an individuals susceptibility to angioedema or angioedema-like events. Such atrial natriuretic factor signal transduction genes include the ANF B-receptor (NPR2).

NPR2 encode atrial natriuretic factor (ANF) B-receptor. NPR2 also has a guanylate cyclase catalytic activity (Nakane, M. & Murad, F,. Adv Pharmacol 26, 7-18 (1994)). ANF degradation is mediated by neutral endopeptidase 24.11 (MME), which is a target of inhibition by Omapatrilat, a vasopeptidase inhibitor. Omapatrilat increases the half life of ANF through this mechanism, which contributes to the blood pressure reduction by this class of drugs in addition of DPC1 inhibition (Weber, M., Am J Hypertens 12, 139S-147S (1999)). Therefore, the inventors of the present invention have conceived that variants NPR2 gene may make an individual hypersensitive to the action of vasopeptidase inhibitors and thus be associated with the incidence of angioedema or angioedema-like events, in general, or in conjunction with the administration of an ACE or vasopeptidase inhibitor.

Single nucleotide polymorphisms in genes that are involved in angiogenesis were also investigated relative to increasing an individuals susceptibility to angioedema or angioedema-like events. Such atrial natriuretic factor signal transduction genes include the following; angiopoietin 1 (ANGPT1), vascular endothelial growth factor (VEGF), vascular endothelial growth factor B (VEGFB), and vascular endothelial growth factor C (VEGFC).

ANGPT1, VEGF, VEGFB and VEGFC all encode molecules involved in angiogenesis. In transgenic mice studies, it was shown that these factors can affect the properties of a blood vessel, including it's susceptibility leakage caused by inflammatory reagents (Thurston, G. et al. Leakage-resistant blood vessels in mice transgenically overexpressing angiopoietin-1. Science 286, 2511-2514 (1999)). The inventors of the present invention have conceived that since angioedema involves a vascular reaction, factors that influence the nature of blood vessel properties may contribute to an individual's susceptibility to angioedema. Therefore, the inventors of the present invention have conceived that variants of the ANGPT1, VEGF, VEGFB, and VEGFC genes may be associated with the incidence of angioedema or angioedema-like events, in general, or in conjunction with the administration of an ACE or vasopeptidase inhibitor.

The invention further provides allele-specific oligonucleotides that hybridize to a gene comprising a single nucleotide polymorphism or to the complement of the gene. Such oligonucleotides will hybridize to one polymorphic form of the nucleic acid molecules described herein but not to the other polymorphic form(s) of the sequence. Thus, such oligonucleotides can be used to determine the presence or absence of particular alleles of the polymorphic sequences described herein. These oligonucleotides can be probes or primers.

The invention further provides a method of analyzing a nucleic acid from an individual. The method determines which base is present at any one of the polymorphic sites shown in Tables I, II, and/or III. Optionally, a set of bases occupying a set of the polymorphic sites shown in Tables I, II, and/or III is determined. This type of analysis can be performed on a number of individuals, who are also tested (previously, concurrently or subsequently) for the presence of a disease phenotype. The presence or absence of disease phenotype is then correlated with a base or set of bases present at the polymorphic site or sites in the individuals tested.

Thus, the invention further relates to a method of predicting the presence, absence, likelihood of the presence or absence, or severity of a particular phenotype or disorder associated with a particular genotype. The method comprises obtaining a nucleic acid sample from an individual and determining the identity of one or more bases (nucleotides) at polymorphic sites of nucleic acid molecules described herein, wherein the presence of a particular base is correlated with a specified phenotype or disorder, thereby predicting the presence, absence, likelihood of the presence or absence, or severity of the phenotype or disorder in the individual. The correlation between a particular polymorphic form of a gene and a phenotype can thus be used in methods of diagnosis of that phenotype, as well as in the development of treatments for the phenotype.

### DEFINITIONS

An oligonucleotide can be DNA or RNA, and single- or double-stranded. Oligonucleotides can be naturally occurring or synthetic, but are typically prepared by synthetic means. Preferred oligonucleotides of the invention include segments of DNA, or their complements, which include any one of the polymorphic sites shown or described in Tables I . The segments can be between and 250 bases, and, in specific embodiments, are between 5-10,5-20, 10-20, 10-50,20-50 or 10-100 bases. For example, the segment can be bases. The polymorphic site can occur within any position of the segment. The segments can be from any of the allelic forms of DNA shown or described in Tables I.

As used herein, the terms “nucleotide”, “base” and “nucleic acid” are intended to be equivalent. The terms “nucleotide sequence”, “nucleic acid sequence”, “nucleic acid molecule” and “segment” are intended to be equivalent.

Hybridization probes are oligonucleotides which bind in a base-specific manner to a complementary strand of nucleic acid. Such probes include peptide nucleic acids, as described in Nielsen et a/., Science 254, 1497-1500 (1991). Probes can be any length suitable for specific hybridization to the target nucleic acid sequence. The most appropriate length of the probe may vary depending upon the hybridization method in which it is being used; for example, particular lengths may be more appropriate for use in microfabricated arrays, while other lengths may be more suitable for use in classical hybridization methods. Such optimizations are known to the skilled artisan. Suitable probes and primers can range from about 12 nucleotides to about 25 nucleotides in length. For example, probes and primers can be 5, 6, 8, 10, 12, 14, 16, 18, 20, 22, 24, 25, 26, or 40 nucleotides in length. The probe or primer preferably overlaps at least one polymorphic site occupied by any of the possible variant nucleotides. The nucleotide sequence can correspond to the coding seqllence of the allele or to the complement of the coding sequence of the allele.

As used herein, the term “primer” refers to a single-stranded oligonucleotide which acts as a point of initiation of template-directed DNA synthesis under appropriate conditions (e.g., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but typically ranges from to nucleotides. Short primer molecules generally require cooler temperatures to form sufficiently stable hybrid complexes with the template. A primer need not reflect the exact sequence of the template, but must be sufficiently complementary to hybridize with a template. The term primer site refers to the area of the target DNA to which a primer hybridizes. The term primer pair refers to a set of primers including a 5′ (upstream) primer that hybridizes with the 5′ end of the DNA sequence to be amplified and a 3′ (downstream) primer that hybridizes with the complement of the 3′ end of the sequence to be amplified.

As used herein, linkage describes the tendency of genes, alleles, loci or genetic markers to be inherited together as a result of their location on the same chromosome. It can be measured by percent recombination between the two genes, alleles, loci or genetic markers.

As used herein, polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild type form. Diploid organisms may be homozygous or heterozygous for allelic forms. A diallelic or biallelic polymorphism has two forms. A triallelic polymorphism has three forms.

Work described herein pertains to the resequencing of large numbers of genes in a large number of individuals to identify polymorphisms which may predispose individuals to disease. For example, polymorphisms in genes which are expressed in liver may predispose individuals to disorders of the liver. Likewise, polymorphisms in genes which are expressed in cardiovascular tissue may predispose individuals to disorders of the heart and/or circulatory system.

By altering amino acid sequence, SNPs may alter the function of the encoded proteins. The discovery of the SNP facilitates biochemical analysis of the variants and the development of assays to characterize the variants and to screen for pharmaceutical that would interact directly with on or another form of the protein. SNPs (including silent SNPs) may also alter the regulation of the gene at the transcriptional or post- transcriptional level. SNPs (including silent SNPs) also enable the development of specific DNA, RNA, or protein-based diagnostics that detect the presence or absence of the polymorphism in particular conditions.

A single nucleotide polymorphism occurs at a polymorphic site occupied by a single nucleotide, which is the site of variation between allelic sequences. The site is usually preceded by_and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic site is occupied by a base other than the reference base. For example, where the reference allele contains the base “T” at the polymorphic site, the altered allele can contain a “C”, “G” or “A” at the polymorphic site.

For the purposes of the present invention the terms “polymorphic position”, “polymorphic site”, “polymorphic locus”, and “polymorphic allele” shall be construed to be equivalent and are defined as the location of a sequence identified as having more than one nucleotide represented at that location in a population comprising at least one or more individuals, and/or chromosomes.

Hybridizations are usually performed under stringent conditions, for example, at a salt concentration of no more than 1 M and a temperature of at least 25° C. For example, conditions of 5×SSPE (750 mM NaCl, mM NaPhosphate, mM EDT A, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

The term “isolated” is used herein to indicate that the material in question exists in a physical milieu distinct from that in which it occurs in nature, and thus is altered “by the hand of man” from its natural state. For example, an isolated nucleic acid of the invention may be substantially isolated with respect to the complex cellular milieu in which it naturally occurs. In some instances, the isolated material will form part of a composition (for example, a crude extract containing other substances), buffer system or reagent mix. In other circumstance, the material may be purified to essential homogeneity, for example as determined by PAGE or column chromatography such as HPLC. Preferably, an isolated nucleic acid comprises at least about 50, 80, or 90 percent (on a molar basis) of all macromolecular species present. For example, an isolated polynucleotide could be part of a vector or a composition of matter, or could be contained within a cell, and still be “isolated” because that vector, composition of matter, or particular cell is not the original environment of the polynucleotide. On one hand, the term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. On the other hand, in consideration of other embodiments of the present invention, specifically the single nucleotide polymorphisms of the present invention, the term “isolated” may refer to genomic or cDNA libraries, whole cell total or mRNA preparations, genomic DNA preparations (including those separated by electrophoresis and transferred onto blots), sheared whole cell genomic DNA preparations. However, the present invention is meant to encompass those compositions where the art demonstrates no distinguishing features of the polynucleotide/sequences of the present invention (e.g., the knowledge that a particular nucleotide position represents a polymorphic site, the knowledge of which allele represents the reference and/or variant nucleotide base, the association of a particular polymorphism with a disease or disorder, wherein such association was not appreciated heretofore, etc.).

One hand, and in specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

On the other hand, and in specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, comprise a portion of non-coding sequences, comprise a portion of an intron sequence, etc., as disclosed herein. In another embodiment, the polynucleotides comprising coding sequences may correspond to a genomic sequence flanking a gene (i.e., 5′ or 3′ to the gene of interest in the genome). In other embodiments, the polynucleotides of the invention may contain the non-coding sequence of more than 1000, 500, 250, 100, 50, 25, 20, 15, 10, 5, 4, 3, 2, or 1 genomic flanking gene(s).

As used herein, a “polynucleotide” refers to a molecule having a nucleic acid sequence contained in SEQ ID NO:X. For example, the polynucleotide can contain the nucleotide sequence of the full length cDNA sequence, including the 5′ and 3′ untranslated sequences, the coding region, with or without a signal sequence, the secreted protein coding region, as well as fragments, epitopes, domains, and variants of the nucleic acid sequence. Moreover, as used herein, a “polypeptide” refers to a molecule having the translated amino acid sequence generated from the polynucleotide as broadly defined.

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were predicted by translation of a DNA sequence determined above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Using the information provided herein, a nucleic acid molecule of the present invention encoding a polypeptide of the present invention may be obtained using standard cloning and screening procedures, such as those for cloning cDNAs using mRNA as starting material.

A “polynucleotide” of the present invention also includes those polynucleotides capable of hybridizing, under stringent hybridization conditions, to sequences described herein, or the complement thereof. “Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65 degree C.

The polynucleotide of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“SEQ ID NO:X” refers to a polynucleotide sequence while “SEQ ID NO:Y” refers to a polypeptide sequence, both sequences identified by an integer specified in Table I, II, and/or III.

“A polypeptide having biological activity” refers to polypeptides exhibiting activity similar, but not necessarily identical to, an activity of a polypeptide of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention.)

The term “organism” as referred to herein is meant to encompass any organism referenced herein, though preferably to eukaryotic organsisms, more preferably to mammals, and most preferably to humans.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

The present invention encompasses the identification of proteins, nucleic acids, or other molecules, that bind to polypeptides and polynucleotides of the present invention (for example, in a receptor-ligand interaction). The polynucleotides of the present invention can also be used in interaction trap assays (such as, for example, that described by Ozenberger and Young (Mol Endocrinol., 9 (10):1321-9, (1995); and Ann. N. Y. Acad. Sci., 7;766:279-81, (1995)).

The polynucleotide and polypeptides of the present invention are useful as probes for the identification and isolation of full-length cDNAs and/or genomic DNA which correspond to the polynucleotides of the present invention, as probes to hybridize and discover novel, related DNA sequences, as probes for positional cloning of this or a related sequence, as probe to “subtract-out” known sequences in the process of discovering other novel polynucleotides, as probes to quantify gene expression, and as probes for microarrays.

In addition, polynucleotides and polypeptides of the present invention may comprise one, two, three, four, five, six, seven, eight, or more membrane domains.

Also, in preferred embodiments the present invention provides methods for further refining the biological function of the polynucleotides and/or polypeptides of the present invention.

Specifically, the invention provides methods for using the polynucleotides and polypeptides of the invention to identify orthologs, homologs, paralogs, variants, and/or allelic variants of the invention. Also provided are methods of using the polynucleotides and polypeptides of the invention to identify the entire coding region of the invention, non-coding regions of the invention, regulatory sequences of the invention, and secreted, mature, pro-, prepro-, forms of the invention (as applicable).

### Polynucleotides and Polypeptides of the Invention

### Features of the Polypeptide Encoded by Gene No:1

The present invention relates to isolated nucleic acid molecules comprising, or alternatively, consisting of all or a portion of one or more variant alleles of the human C1S gene (e.g., wherein reference or wildtype allele is exemplified by a “G” at nucleotide 558; a “C” at nucleotide 643; a “T” at nucleotide 982; a “C” at nucleotide 1122; a “G” at nucleotide 1136; and/or a “C” at nucleotide 1879 of SEQ ID NO:883). Preferred portions are at least 10, preferably at least 20, preferably at least 40, preferably at least 100, contiguous polynucleotides and comprise one or more reference alleles at the nucleotide position(s) provided in Table I, II, or III.

The present invention relates to isolated nucleic acid molecules comprising, or alternatively, consisting of all or a portion of one or more variant alleles of the human C1S gene (e.g., wherein alternate or variant allele is exemplified by an “A” at nucleotide 558; a “T” at nucleotide 643; a “A” at nucleotide 982; a “T” at nucleotide 1122; a “A” at nucleotide 1136; and/or a “T” at nucleotide 1879 of SEQ ID NO:883). Preferred portions are at least 10, preferably at least 20, preferably at least 40, preferably at least 100, contiguous polynucleotides and comprise one or more alternate (or variant) allele(s) at the nucleotide position(s) provided in Table I, II, or III. The invention further relates to isolated gene products, e.g., polypeptides and/or proteins, which are encoded by a nucleic acid molecule comprising all or a portion of at least one or more variant allele(s) of the gene.

In one embodiment, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with one or more reference allele(s) at the nucleotide position(s) provided in Table I, II, or III for C1S gene (or diagnosing or aiding in the diagnosis of such a disorder) comprising the steps of obtaining a DNA sample from an individual to be assessed and determining the nucleotide present at said nucleotide position. The presence of the reference allele at said position indicates that the individual has a greater likelihood of having a disorder associated therewith than an individual having the alternate (variant) allele(s) at said position(s), or a greater likelihood of having more severe symptoms.

Conversely, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with the alternate (variant) allele(s) at the nucleotide position provided in Table I, II, or III for the C1S gene (or diagnosing or aiding in the diagnosis of such a disorder) comprising the steps of obtaining a DNA sample from an individual to be assessed and determining the nucleotide present at said nucleotide position. The presence of the alternate (variant) allele(s) at said position(s) indicates that the individual has a greater likelihood of having a disorder associated therewith than an individual having the reference allele(s) at said position(s), or a greater likelihood of having more severe symptoms.

The present invention further relates to isolated proteins or polypeptides comprising, or alternatively, consisting of all or a portion of the encoded variant amino acid sequence of the human C1S polypeptide (e.g., wherein reference or wildtype human complement component 1, s subcomponent protein polypeptide is exemplified by a “R” at amino acid 119; a “N” at amino acid 260; an “A” at amino acid 307; and/or a “V” at amino acid 312 of SEQ ID NO:884). Preferred portions are at least 10, preferably at least 20, preferably at least 40, preferably at least 100, contiguous polypeptides and comprises the reference amino acid allele at the amino acid position provided in Table I, II, or III of the C1S polypeptide. The invention further relates to isolated nucleic acid molecules encoding such polypeptides or proteins, as well as to antibodies that bind to such proteins or polypeptides.

The present invention further relates to isolated proteins or polypeptides comprising, or alternatively, consisting of all or a portion of the encoded variant amino acid sequence of the human C1S polypeptide (e.g., wherein alternate or variant human complement component 1, s subcomponent protein polypeptide is exemplified by a “H” at amino acid 119; a “K” at amino acid 260; an “V” at amino acid 307; and/or a “I” at amino acid 312 of SEQ ID NO:884). Preferred portions are at least 10, preferably at least 20, preferably at least 40, preferably at least 100, contiguous polypeptides and comprises the alternate amino acid allele at the amino acid position provided in Table I, II, or III of the C1S polypeptide. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the SNPs and methods of the present invention include, the following, non-limiting diseases and disorders: angioedema, susceptibility to acquiring an angioedema or an angioedema-like disorder upon the administration of a pharmaceutically acceptable amount of an ACE inhibitor and/or vasopeptidase inhibitor cardiovascular disease; angina pectoris, hypertension, heart failure, myocardial infarction, ventricular hypertrophy, cough associated with ACE inhibitors, cough associated with vasopeptidase inhibitors, vascular diseases, miscrovascular disease, vascular leak syndrome, aneurysm, stroke, embolism, thrombosis, endothelial dysfunction, coronary artery disease, arteriosclerosis, and/or atherosclerosis.

Additional disorders which may be detected, diagnosed, identified, treated, prevented, and/or ameliorated by the SNPs and methods of the present invention include, the following, non-limiting diseases and disorders: hypotensive reactions during blood transfusions (Transfusion. October 1999;39 (10):1084-8.), hypersensitivity reactions during hemodialysis (Peptides. 1999;20 (4):421-30.), sepsis, inflammatory arthritis, and enterocolitis (Clin Rev Allergy Immunol. 1998 Winter;16 (4):365-84.), enterocolitis (Gut. September 1998;43 (3):365-74.), chronic granulomatous intestinal and systemic inflammation (FASEB J. March 1998;12 (3):325-33.), peptidoglycan-induced arthritis (Arthritis Rheum. July 1997;40 (7):1327-33.), arthritis (Proc Assoc Am Physicians. January 1997;109 (1):10-22.), intestinal inflammation (Dig Dis Sci. May1996; 41 (5):912-20.), acute phase response of inflammation (Peptides. 1996;17 (7):1163-70.), asthma (J Appl Physiol 1995; 78: 1844-1852), chronic obstructive pulmonary disease (COPD), cough reflex, allergies, and/or neurogenic inflammation.

Additional disorders which may be detected, diagnosed, identified, treated, prevented, and/or ameliorated by the SNPs and methods of the present invention include, the following, non-limiting diseases and disorders: hypotensive reactions during blood transfusions (Transfusion. October 1999;39 (10):1084-8.), hypersensitivity reactions during hemodialysis (Peptides. 1999;20 (4):421-30.), sepsis, inflammatory arthritis, and enterocolitis (Clin Rev Allergy Immunol. 1998 Winter;16 (4):365-84.), enterocolitis (Gut. September 1998;43 (3):365-74.), chronic granulomatous intestinal and systemic inflammation (FASEB J. March 1998;12 (3):325-33.), peptidoglycan-induced arthritis (Arthritis Rheum. July 1997;40 (7):1327-33.), arthritis (Proc Assoc Am Physicians. January 1997;109 (1):10-22.), intestinal inflammation (Dig Dis Sci. May 1996; 41 (5):912-20.), acute phase response of inflammation (Peptides. 1996;17 (7):1163-70.), asthma (J Appl Physiol 1995; 78: 1844-1852), chronic obstructive pulmonary disease (COPD), cough reflex, allergies, and/or neurogenic inflammation.

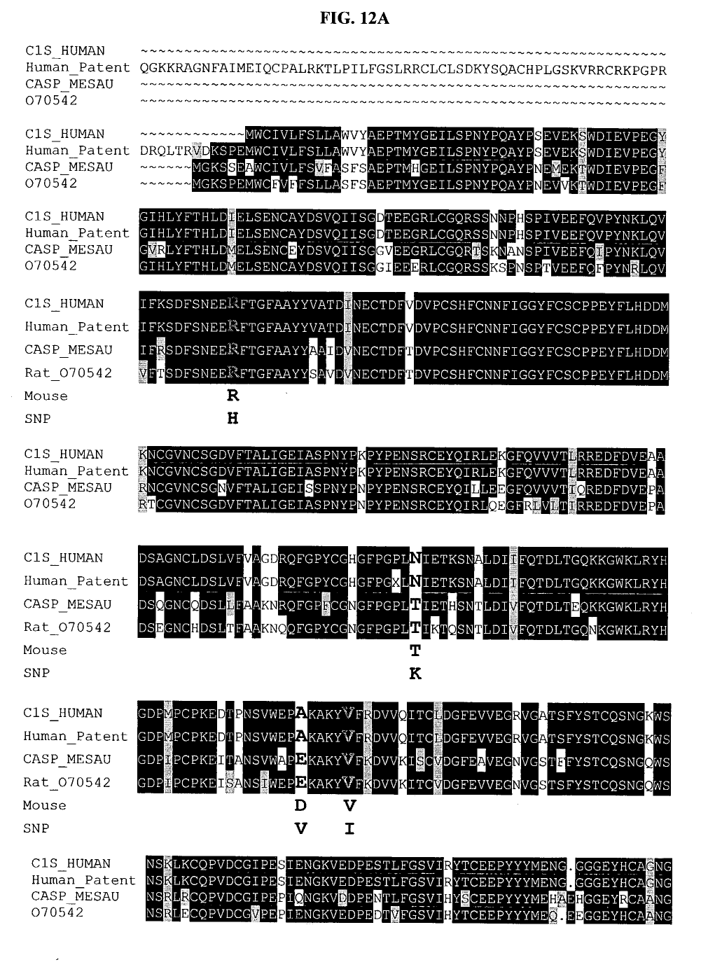

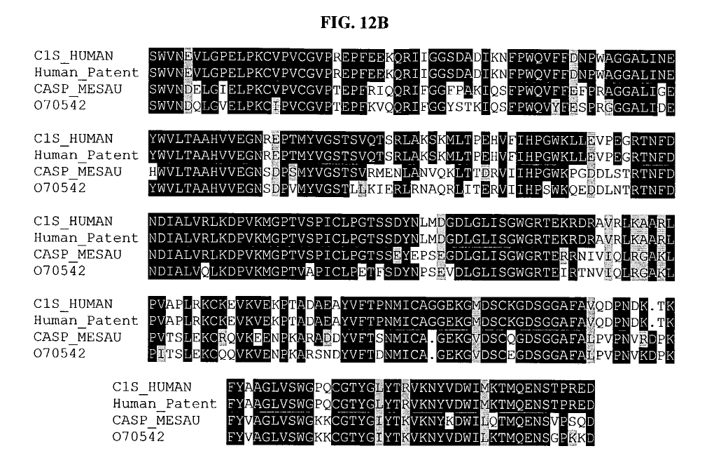

Analysis of the ANPEP coding SNPs of the present invention led to the determination that some of these SNPs lie within conserved domains of the ANPEP polypeptide and may modulate its physiological function. Specifically, the 119 R/H (SNP ID NO: AE111s17) resides in the CUB domain, a domain present in developmentally regulated domain of the C1S protein. This position is conserved in different organisms including mouse not given in the alignment. A change from “R to H”, though both are basic in nature, could be important due to the fact that Histidine is a bulky amino acid and also it corresponds to a conserved position; the 260 N/K (SNP ID NO: AE111s20) resides in the CUB domain. This position is not conserved in the alignment (N or T; mouse and rat sequences have a “T” in this position) but the substitution is conservative since both N and K are basic in nature; the 307 A/V variant (SNP ID NO: AE111s21) resides in the Sushi domain, a domain present in complement control protein, of the C1S protein. This position is not conserved in the alignment (amino acids corresponding to A, E and D are present in this position) but the substitution is conservative since both A and V are small hydrophobic amino acids; and the 312 V/I variant (SNP ID NO: AE111s22) resides in the Sushi domain. This represents a conserved residue in the alignment but the substitution from V to I is conservative.. An alignment of the ANPEP polypeptide with ANPEP sequences from other species is provided in FIGS. 12A-B.

### Features of the Polypeptide Encoded by Gene No:2

The present invention relates to isolated nucleic acid molecules comprising, or alternatively, consisting of all or a portion of one or more variant alleles of the human ANPEP gene (e.g., wherein reference or wildtype allele is exemplified by a “T” at nucleotide 2773; a “C” at nucleotide 2664; a “C” at nucleotide 2553; a “C” at nucleotide 2519; a “C” at nucleotide 2505; an “A” at nucleotide 1929; a “T” at nucleotide 1928; a “C” at nucleotide 1227; a “G” at nucleotide 1083; a “G” at nucleotide 1074; a “C” at nucleotide 1052; a “T” at nucleotide 747; and/or a “C” at nucleotide 474 of SEQ ID NO:885). Preferred portions are at least 10, preferably at least 20, preferably at least 40, preferably at least 100, contiguous polynucleotides and comprise one or more reference alleles at the nucleotide position(s) provided in Table I, II, or III.

The present invention relates to isolated nucleic acid molecules comprising, or alternatively, consisting of all or a portion of one or more variant alleles of the human ANPEP gene (e.g., wherein alternate or variant allele is exemplified by a “C” at nucleotide 2773; an “A” at nucleotide 2664; a “T” at nucleotide 2553; a “T” at nucleotide 2519; a “T” at nucleotide 2505; a “G” at nucleotide 1929; an “A” at nucleotide 1928; a “T” at nucleotide 1227; an “A” at nucleotide 1083; a “T” at nucleotide 1074; a “T” at nucleotide 1052; an “A” at nucleotide 747; and/or a “T” at nucleotide 474 of SEQ ID NO:885). Preferred portions are at least 10, preferably at least 20, preferably at least 40, preferably at least 100, contiguous polynucleotides and comprise one or more alternate (or variant) allele(s) at the nucleotide position(s) provided in Table I, II, or III. The invention further relates to isolated gene products, e.g., polypeptides and/or proteins, which are encoded by a nucleic acid molecule comprising all or a portion of at least one or more variant allele(s) of the gene.

In one embodiment, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with one or more reference allele(s) at the nucleotide position(s) provided in Table I, II, or III for ANPEP gene (or diagnosing or aiding in the diagnosis of such a disorder) comprising the steps of obtaining a DNA sample from an individual to be assessed and determining the nucleotide present at said nucleotide position. The presence of the reference allele at said position indicates that the individual has a greater likelihood of having a disorder associated therewith than an individual having the alternate (variant) allele(s) at said position(s), or a greater likelihood of having more severe symptoms.

Conversely, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with the alternate (variant) allele(s) at the nucleotide position provided in Table I, II, or III for the ANPEP gene (or diagnosing or aiding in the diagnosis of such a disorder) comprising the steps of obtaining a DNA sample from an individual to be assessed and determining the nucleotide present at said nucleotide position. The presence of the alternate (variant) allele(s) at said position(s) indicates that the individual has a greater likelihood of having a disorder associated therewith than an individual having the reference allele(s) at said position(s), or a greater likelihood of having more severe symptoms.

The present invention further relates to isolated proteins or polypeptides comprising, or alternatively, consisting of all or a portion of the encoded variant amino acid sequence of the human ANPEP polypeptide (e.g., wherein reference or wildtype human alanyl (membrane) aminopeptidase protein polypeptide is exemplified by a “A” at amino acid 800; an “I” at amino acid 603; and/or an “A” at amino acid 311 of SEQ ID NO:886). Preferred portions are at least 10, preferably at least 20, preferably at least 40, preferably at least 100, contiguous polypeptides and comprises the reference amino acid allele at the amino acid position provided in Table I, II, or III of the ANPEP polypeptide. The invention further relates to isolated nucleic acid molecules encoding such polypeptides or proteins, as well as to antibodies that bind to such proteins or polypeptides.

The present invention further relates to isolated proteins or polypeptides comprising, or alternatively, consisting of all or a portion of the encoded variant amino acid sequence of the human ANPEP polypeptide (e.g., wherein alternate or variant human alanyl (membrane) aminopeptidase protein polypeptide is exemplified by a “V” at amino acid 800; a “M” at amino acid 603; a “K” at amino acid 603; and/or a “V” at amino acid 311 of SEQ ID NO:886). Preferred portions are at least 10, preferably at least 20, preferably at least 40, preferably at least 100, contiguous polypeptides and comprises the alternate amino acid allele at the amino acid position provided in Table I, II, or III of the ANPEP polypeptide. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the SNPs and methods of the present invention include, the following, non-limiting diseases and disorders: angioedema, susceptibility to acquiring an angioedema or an angioedema-like disorder upon the administration of a pharmaceutically acceptable amount of an ACE inhibitor and/or vasopeptidase inhibitor cardiovascular disease; angina pectoris, hypertension, heart failure, myocardial infarction, ventricular hypertrophy, cough associated with ACE inhibitors, cough associated with vasopeptidase inhibitors, vascular diseases, miscrovascular disease, vascular leak syndrome, aneurysm, stroke, embolism, thrombosis, endothelial dysfunction, coronary artery disease, arteriosclerosis, and/or atherosclerosis.

Additional disorders which may be detected, diagnosed, identified, treated, prevented, and/or ameliorated by the SNPs and methods of the present invention include, the following, non-limiting diseases and disorders: hypotensive reactions during blood transfusions (Transfusion. October 1999;39 (10):1084-8.), hypersensitivity reactions during hemodialysis (Peptides. 1999;20 (4):421-30.), sepsis, inflammatory arthritis, and enterocolitis (Clin Rev Allergy Immunol. 1998 Winter;16 (4):365-84.), enterocolitis (Gut. September 1998;43 (3):365-74.), chronic granulomatous intestinal and systemic inflammation (FASEB J. March 1998;12 (3):325-33.), peptidoglycan-induced arthritis (Arthritis Rheum. July 1997;40 (7):1327-33.), arthritis (Proc Assoc Am Physicians. January 1997;109 (1):10-22.), intestinal inflammation (Dig Dis Sci. May 1996; 41 (5):912-20.), acute phase response of inflammation (Peptides. 1996;17 (7):1163-70.), asthma (J Appl Physiol 1995; 78: 1844-1852), chronic obstructive pulmonary disease (COPD), cough reflex, allergies, and/or neurogenic inflammation.

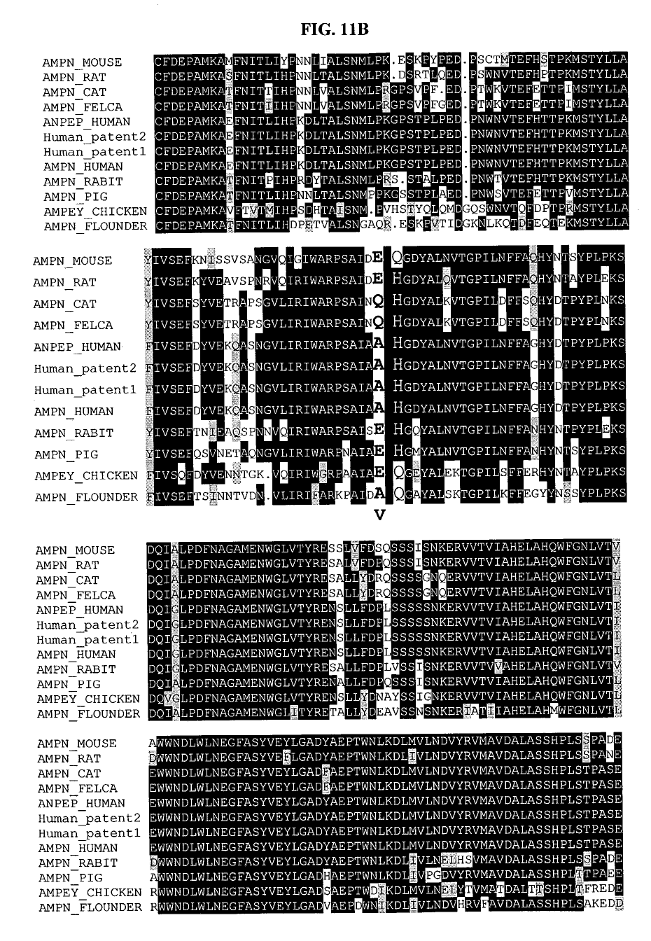

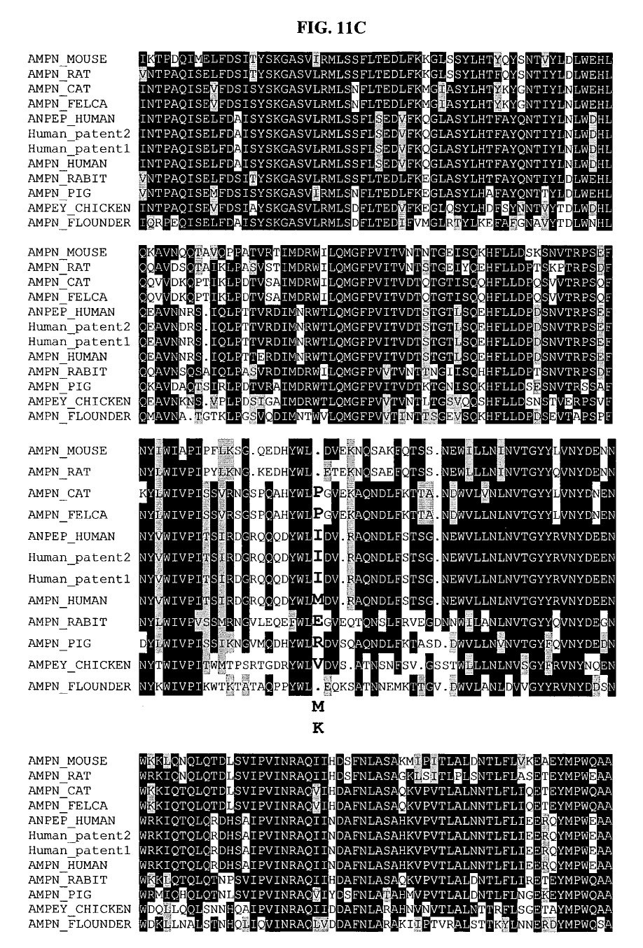

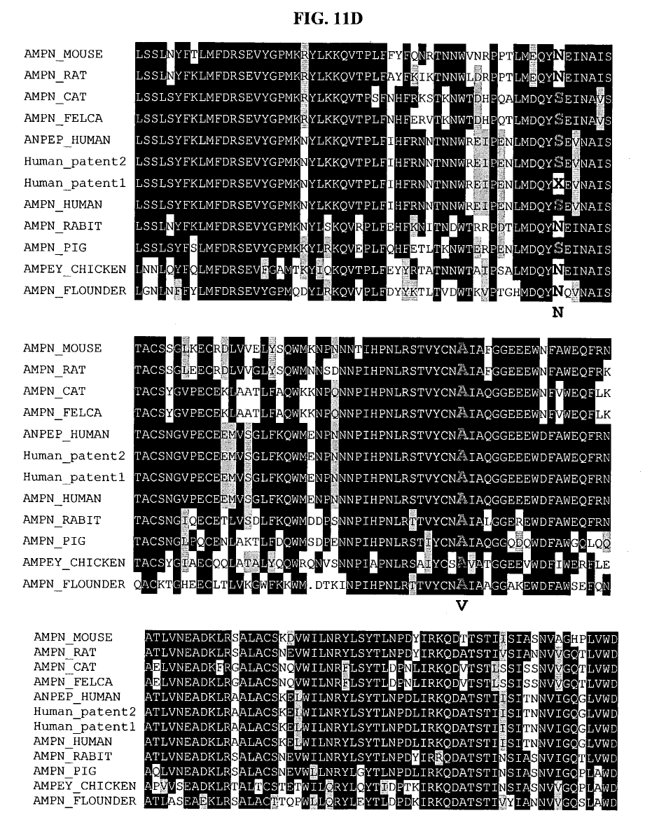

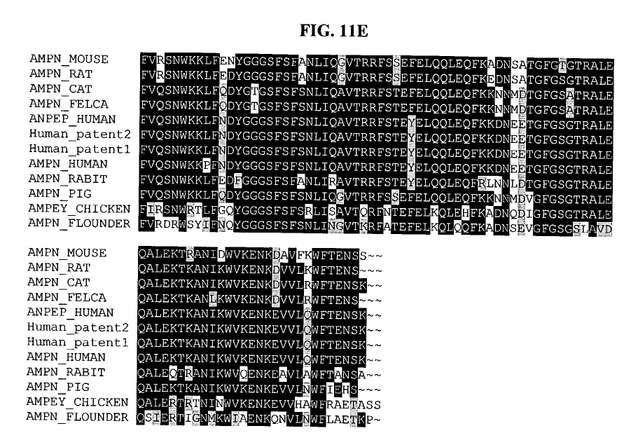

Analysis of the ANPEP coding SNPs of the present invention led to the determination that some of these SNPs lie within conserved domains of the ANPEP polypeptide and may modulate its physiological function. Specifically, the 86 Q/R variant residues in the Peptidase domain of the ANPEP protein. The change from Q to R is conservative and there are known rodent and other sequences which have R and N in this position; the 311 A/V variant (SNP ID NO: AE112s61) residues in the Peptidase domain of the ANPEP protein. The amino acids corresponding to this position are Glutamine and Aspartic acid in other organisms. In humans, it is Alanine and it is very different from other organisms. Alanine and Valine are a conservative substitutions and it maintains the difference between human and other organism sequences; the 603 I/M/K variant (SNP ID NO: AE112s18 and AE112s19) does not reside in a conserved location and it has different amino acids such as P, I, M, E, R, V. The change corresponding to M is already documented in one of the human sequences and the change corresponding to K is novel but may related to “R” found in the Pig AMP-N sequence; the 752 S/N variant does not residue in a conserved position; and the 800 A/V variant (SNP ID NO: AE112s32) residues in a highly conserved position among the different organisms. Therefore, the change from A to V, though a conservative hydrophobic substitution, could be important in the function of the ANPEP protein. An alignment of the ANPEP polypeptide with ANPEP sequences from other species is provided in FIGS. 11A-E.

### Features of the Polypeptide Encoded by Gene No:3

The present invention relates to isolated nucleic acid molecules comprising, or alternatively, consisting of all or a portion of one or more variant alleles of the human MEP1B gene (e.g., wherein reference or wildtype allele is exemplified by a “G” at nucleotide 196; an “A” at nucleotide 394; an “A” at nucleotide 838; a “G” at nucleotide 1022; a “T” at nucleotide 1268; a “G” at nucleotide 1315; an “A” at nucleotide 1740; a “C” at nucleotide 1741; a “C” at nucleotide 1999; and/or a “T” at nucleotide 2130 of SEQ ID NO:887). Preferred portions are at least 10, preferably at least 20, preferably at least 40, preferably at least 100, contiguous polynucleotides and comprise one or more reference alleles at the nucleotide position(s) provided in Table I, II, or III.

The present invention relates to isolated nucleic acid molecules comprising, or alternatively, consisting of all or a portion of one or more variant alleles of the human MEP1B gene (e.g., wherein alternate or variant allele is exemplified by an “A” at nucleotide 196; a “G” at nucleotide 394; a “G” at nucleotide 838; an “A” at nucleotide 1022; an “A” at nucleotide 1268; an “A” at nucleotide 1315; a “C” at nucleotide 1740; a “T” at nucleotide 1741; a “T” at nucleotide 1999; and/or a “C” at nucleotide 2130 of SEQ ID NO:887). Preferred portions are at least 10, preferably at least 20, preferably at least 40, preferably at least 100, contiguous polynucleotides and comprise one or more alternate (or variant) allele(s) at the nucleotide position(s) provided in Table I, II, or III. The invention further relates to isolated gene products, e.g., polypeptides and/or proteins, which are encoded by a nucleic acid molecule comprising all or a portion of at least one or more variant allele(s) of the gene.

In one embodiment, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with one or more reference allele(s) at the nucleotide position(s) provided in Table I, II, or III for MEP1B gene (or diagnosing or aiding in the diagnosis of such a disorder) comprising the steps of obtaining a DNA sample from an individual to be assessed and determining the nucleotide present at said nucleotide position. The presence of the reference allele at said position indicates that the individual has a greater likelihood of having a disorder associated therewith than an individual having the alternate (variant) allele(s) at said position(s), or a greater likelihood of having more severe symptoms.

Conversely, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with the alternate (variant) allele(s) at the nucleotide position provided in Table I, II, or III for the MEP1B gene (or diagnosing or aiding in the diagnosis of such a disorder) comprising the steps of obtaining a DNA sample from an individual to be assessed and determining the nucleotide present at said nucleotide position. The presence of the alternate (variant) allele(s) at said position(s) indicates that the individual has a greater likelihood of having a disorder associated therewith than an individual having the reference allele(s) at said position(s), or a greater likelihood of having more severe symptoms.

The present invention further relates to isolated proteins or polypeptides comprising, or alternatively, consisting of all or a portion of the encoded variant amino acid sequence of the human MEP1B polypeptide (e.g., wherein reference or wildtype human meprin A, beta protein polypeptide is exemplified by an “I” at amino acid 115; a “V” at amino acid 326; a “S” at amino acid 408; a “H” at amino acid 565; and/or a “L” at amino acid 695 of SEQ ID NO:888). Preferred portions are at least 10, preferably at least 20, preferably at least 40, preferably at least 100, contiguous polypeptides and comprises the reference amino acid allele at the amino acid position provided in Table I, II, or III of the MEP1B polypeptide. The invention further relates to isolated nucleic acid molecules encoding such polypeptides or proteins, as well as to antibodies that bind to such proteins or polypeptides.

The present invention further relates to isolated proteins or polypeptides comprising, or alternatively, consisting of all or a portion of the encoded variant amino acid sequence of the human MEP1B polypeptide (e.g., wherein alternate or variant human meprin A, beta protein polypeptide is exemplified by a “M” at amino acid 115; a “M” at amino acid 326; a “T” at amino acid 408; a “P” at amino acid 565; and/or a “P” at amino acid 695 of SEQ ID NO:888). Preferred portions are at least 10, preferably at least 20, preferably at least 40, preferably at least 100, contiguous polypeptides and comprises the alternate amino acid allele at the amino acid position provided in Table I, II, or III of the MEP1B polypeptide. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the SNPs and methods of the present invention include, the following, non-limiting diseases and disorders: angioedema, susceptibility to acquiring an angioedema or an angioedema-like disorder upon the administration of a pharmaceutically acceptable amount of an ACE inhibitor and/or vasopeptidase inhibitor cardiovascular disease; angina pectoris, hypertension, heart failure, myocardial infarction, ventricular hypertrophy, cough associated with ACE inhibitors, cough associated with vasopeptidase inhibitors, vascular diseases, miscrovascular disease, vascular leak syndrome, aneurysm, stroke, embolism, thrombosis, endothelial dysfunction, coronary artery disease, arteriosclerosis, and/or atherosclerosis.

Additional disorders which may be detected, diagnosed, identified, treated, prevented, and/or ameliorated by the SNPs and methods of the present invention include, the following, non-limiting diseases and disorders: hypotensive reactions during blood transfusions (Transfusion. October 1999;39 (10):1084-8.), hypersensitivity reactions during hemodialysis (Peptides. 1999;20 (4):421-30.), sepsis, inflammatory arthritis, and enterocolitis (Clin Rev Allergy Immunol. 1998 Winter;16 (4):365-84.), enterocolitis (Gut. September 1998;43 (3):365-74.), chronic granulomatous intestinal and systemic inflammation (FASEB J. March 1998;12 (3):325-33.), peptidoglycan-induced arthritis (Arthritis Rheum. July 1997;40 (7):1327-33.), arthritis (Proc Assoc Am Physicians. January 1997;109 (1):10-22.), intestinal inflammation (Dig Dis Sci. May 1996; 41 (5):912-20.), acute phase response of inflammation (Peptides. 1996;17 (7):1163-70.), asthma (J Appl Physiol 1995; 78: 1844-1852), chronic obstructive pulmonary disease (COPD), cough reflex, allergies, and/or neurogenic inflammation.

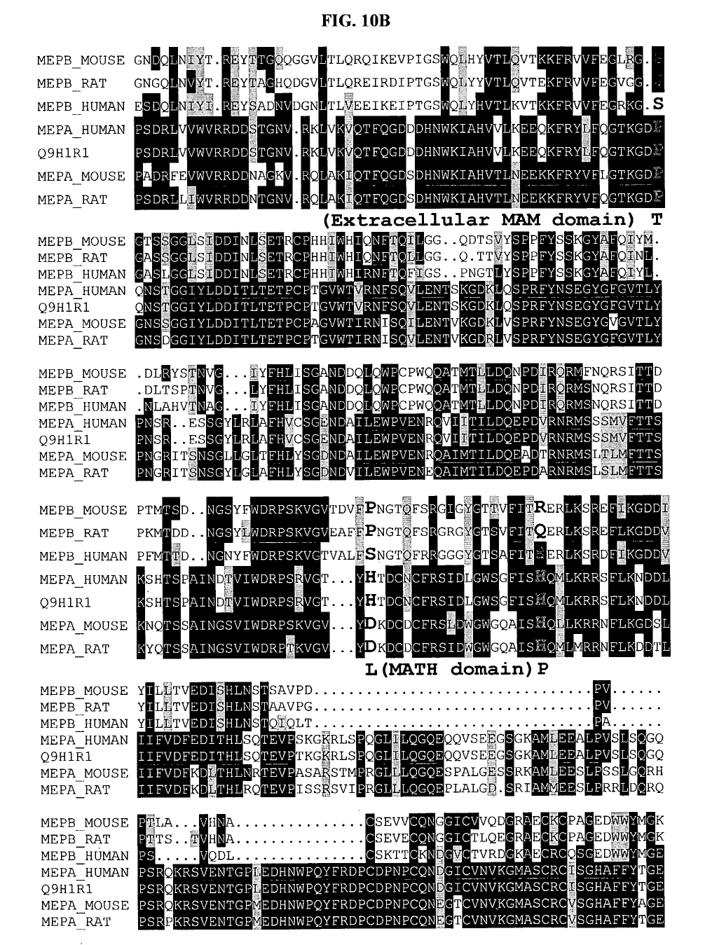

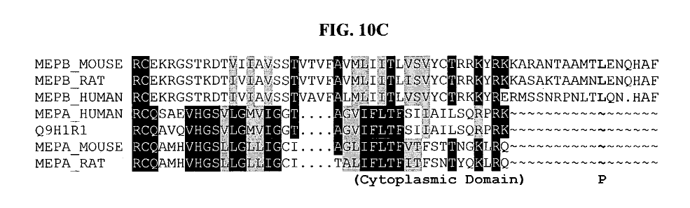

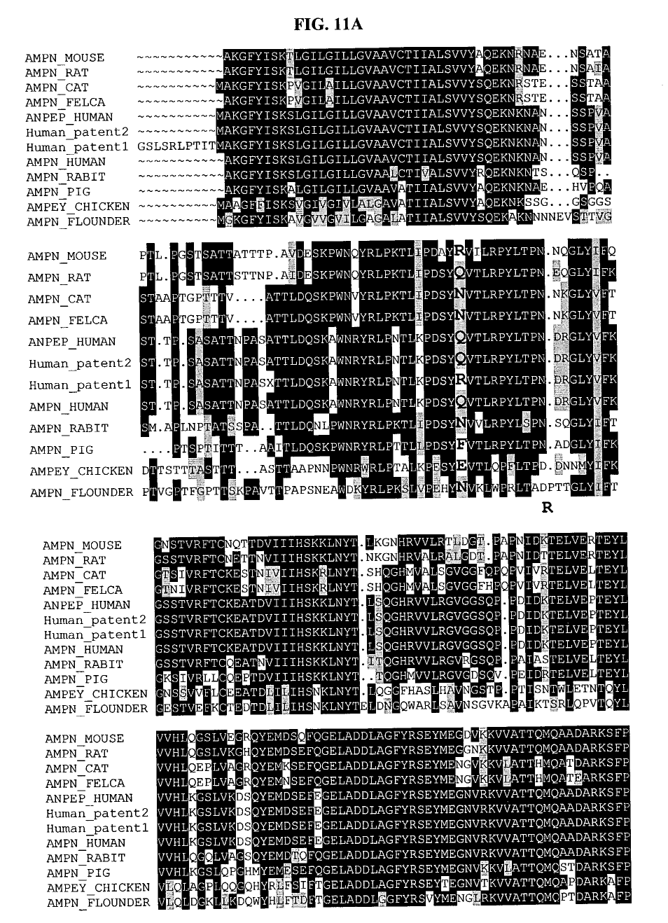

Analysis of the MEP1B coding SNPs of the present invention led to the determination that some of these SNPs lie within conserved domains of the MEP1B polypeptide and may modulate its physiological function. Specifically, the AA116:I/M variant (SNP ID NO: AE113s38) resides within the metalloprotease domain and is a conserved residue among MEPRIN B and A sequences; the AA326:V/M variant (SNP ID NO: AE113s6) resides within the extracellular MAM domain and this substitution may be favorable in that it makes the human MEPB sequence identical to rodent sequences at this position; the AA408:S/T variant (SNP ID NO: AE113s9) resides within the extracellular MAM domain and represents a conservative substitution, although its functional role is not clear; the AA546:S/L variant resides in a domain called MATH which is present in Meprin proteins and may be significant due to the substitution from hydrophilic S to hydrophobic L; the AA565:H/P variant (SNP ID NO: AE113s18) resides in a domain called MATH which is present in Meprin proteins. While this residue is not conserved, all the amino acids in this position are basic in nature. Therefore, the change to Proline could be significant both in terms of non-basicity and conformational change; and the AA695:L/P variant—(SNP ID NO: AE113s25) resides in the cytoplasmic tail of the MEPB1 protein. It is a conserved residue in rodent MEPB sequences. Mutagenesis of this residue (AA685 Tyrosine to Proline) results in a transport incompetent protein and retention of the protein in the Endoplasmic Reticulum. The introduction of an additional proline in the region may affect the protein conformation further and potentially prevent the MEPRIN-beta protein from reaching its functional Location. The C-terminus of the MEPB1 protein in thought to play an important role in intracellular trafficking. An alignment of these SNPs with other metrin proteins in provided in FIGS. 10A-C.

### Features of the Polypeptide Encoded by Gene No:4

The present invention relates to isolated nucleic acid molecules comprising, or alternatively, consisting of all or a portion of one or more variant alleles of the human XPNPEPL gene (e.g., wherein reference or wildtype allele is exemplified by a “C” at nucleotide 1773; and/or a “C” at nucleotide 225 of SEQ ID NO:889). Preferred portions are at least 10, preferably at least 20, preferably at least 40, preferably at least 100, contiguous polynucleotides and comprise one or more reference alleles at the nucleotide position(s) provided in Table I, II, or III.

The present invention relates to isolated nucleic acid molecules comprising, or alternatively, consisting of all or a portion of one or more variant alleles of the human XPNPEPL gene (e.g., wherein alternate or variant allele is exemplified by a “G” at nucleotide 1773; and/or a “T” at nucleotide 225 of SEQ ID NO:889). Preferred portions are at least 10, preferably at least 20, preferably at least 40, preferably at least 100, contiguous polynucleotides and comprise one or more alternate (or variant) allele(s) at the nucleotide position(s) provided in Table I, II, or III. The invention further relates to isolated gene products, e.g., polypeptides and/or proteins, which are encoded by a nucleic acid molecule comprising all or a portion of at least one or more variant allele(s) of the gene.

In one embodiment, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with one or more reference allele(s) at the nucleotide position(s) provided in Table I, I1, or III for XPNPEPL gene (or diagnosing or aiding in the diagnosis of such a disorder) comprising the steps of obtaining a DNA sample from an individual to be assessed and determining the nucleotide present at said nucleotide position. The presence of the reference allele at said position indicates that the individual has a greater likelihood of having a disorder associated therewith than an individual having the alternate (variant) allele(s) at said position(s), or a greater likelihood of having more severe symptoms.

Conversely, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with the alternate (variant) allele(s) at the nucleotide position provided in Table I, II, or III for the XPNPEPL gene (or diagnosing or aiding in the diagnosis of such a disorder) comprising the steps of obtaining a DNA sample from an individual to be assessed and determining the nucleotide present at said nucleotide position. The presence of the alternate (variant) allele(s) at said position(s) indicates that the individual has a greater likelihood of having a disorder associated therewith than an individual having the reference allele(s) at said position(s), or a greater likelihood of having more severe symptoms.

Representative disorders which may be detected, diagnosed, identified, treated, prevented, and/or ameliorated by the SNPs and methods of the present invention include, the following, non-limiting diseases and disorders: angioedema, susceptibility to acquiring an angioedema or an angioedema-like disorder upon the administration of a pharmaceutically acceptable amount of an ACE inhibitor and/or vasopeptidase inhibitor cardiovascular disease; angina pectoris, hypertension, heart failure, myocardial infarction, ventricular hypertrophy, cough associated with ACE inhibitors, cough associated with vasopeptidase inhibitors, vascular diseases, miscrovascular disease, vascular leak syndrome, aneurysm, stroke, embolism, thrombosis, endothelial dysfunction, coronary artery disease, arteriosclerosis, and/or atherosclerosis.

Additional disorders which may be detected, diagnosed, identified, treated, prevented, and/or ameliorated by the SNPs and methods of the present invention include, the following, non-limiting diseases and disorders: hypotensive reactions during blood transfusions (Transfusion. October 1999;39 (10):1084-8.), hypersensitivity reactions during hemodialysis (Peptides. 1999;20 (4):421-30.), sepsis, inflammatory arthritis, and enterocolitis (Clin Rev Allergy Immunol. 1998 Winter;16 (4):365-84.), enterocolitis (Gut. September 1998;43 (3):365-74.), chronic granulomatous intestinal and systemic inflammation (FASEB J. March 1998;12 (3):325-33.), peptidoglycan-induced arthritis (Arthritis Rheum. July 1997;40 (7):1327-33.), arthritis (Proc Assoc Am Physicians. January 1997;109 (1):10-22.), intestinal inflammation (Dig Dis Sci. May 1996; 41 (5):912-20.), acute phase response of inflammation (Peptides. 1996;17 (7):1163-70.), asthma (J Appl Physiol 1995; 78: 1844-1852), chronic obstructive pulmonary disease (COPD), cough reflex, allergies, and/or neurogenic inflammation.

### Features of the Polypeptide Encoded by Gene No:5

The present invention relates to isolated nucleic acid molecules comprising, or alternatively, consisting of all or a portion of one or more variant alleles of the human KLK1 gene (e.g., wherein reference or wildtype allele is exemplified by a “C” at nucleotide 603 of SEQ ID NO:891). Preferred portions are at least 10, preferably at least 20, preferably at least 40, preferably at least 100, contiguous polynucleotides and comprise one or more reference alleles at the nucleotide position(s) provided in Table I, II, or III.

The present invention relates to isolated nucleic acid molecules comprising, or alternatively, consisting of all or a portion of one or more variant alleles of the human KLK1gene (e.g., wherein alternate or variant allele is exemplified by a “T” at nucleotide 603 of SEQ ID NO:891). Preferred portions are at least 10, preferably at least 20, preferably at least 40, preferably at least 100, contiguous polynucleotides and comprise one or more alternate (or variant) allele(s) at the nucleotide position(s) provided in Table I, II, or III. The invention further relates to isolated gene products, e.g., polypeptides and/or proteins, which are encoded by a nucleic acid molecule comprising all or a portion of at least one or more variant allele(s) of the gene.

In one embodiment, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with one or more reference allele(s) at the nucleotide position(s) provided in Table I, II, or III for KLK1 gene (or diagnosing or aiding in the diagnosis of such a disorder) comprising the steps of obtaining a DNA sample from an individual to be assessed and determining the nucleotide present at said nucleotide position. The presence of the reference allele at said position indicates that the individual has a greater likelihood of having a disorder associated therewith than an individual having the alternate (variant) allele(s) at said position(s), or a greater likelihood of having more severe symptoms.

Conversely, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with the alternate (variant) allele(s) at the nucleotide position provided in Table I, II, or III for the KLK1 gene (or diagnosing or aiding in the diagnosis of such a disorder) comprising the steps of obtaining a DNA sample from an individual to be assessed and determining the nucleotide present at said nucleotide position. The presence of the alternate (variant) allele(s) at said position(s) indicates that the individual has a greater likelihood of having a disorder associated therewith than an individual having the reference allele(s) at said position(s), or a greater likelihood of having more severe symptoms.

Representative disorders which may be detected, diagnosed, identified, treated, prevented, and/or ameliorated by the SNPs and methods of the present invention include, the following, non-limiting diseases and disorders: angioedema, susceptibility to acquiring an angioedema or an angioedema-like disorder upon the administration of a pharmaceutically acceptable amount of an ACE inhibitor and/or vasopeptidase inhibitor cardiovascular disease; angina pectoris, hypertension, heart failure, myocardial infarction, ventricular hypertrophy, cough associated with ACE inhibitors, cough associated with vasopeptidase inhibitors, vascular diseases, miscrovascular disease, vascular leak syndrome, aneurysm, stroke, embolism, thrombosis, endothelial dysfunction, coronary artery disease, arteriosclerosis, and/or atherosclerosis.

Additional disorders which may be detected, diagnosed, identified, treated, prevented, and/or ameliorated by the SNPs and methods of the present invention include, the following, non-limiting diseases and disorders: hypotensive reactions during blood transfusions (Transfusion. October 1999;39 (10):1084-8.), hypersensitivity reactions during hemodialysis (Peptides. 1999;20 (4):421-30.), sepsis, inflammatory arthritis, and enterocolitis (Clin Rev Allergy Immunol. 1998 Winter;16 (4):365-84.), enterocolitis (Gut. September 1998;43 (3):365-74.), chronic granulomatous intestinal and systemic inflammation (FASEB J. March 1998;12 (3):325-33.), peptidoglycan-induced arthritis (Arthritis Rheum. July 1997;40 (7):1327-33.), arthritis (Proc Assoc Am Physicians. January 1997;109 (1):10-22.), intestinal inflammation (Dig Dis Sci. May 1996; 41 (5):912-20.), acute phase response of inflammation (Peptides. 1996;17 (7):1163-70.), asthma (J Appl Physiol 1995; 78: 1844-1852), chronic obstructive pulmonary disease (COPD), cough reflex, allergies, and/or neurogenic inflammation.

### Features of the Polypeptide Encoded by Gene No:6

The present invention relates to isolated nucleic acid molecules comprising, or alternatively, consisting of all or a portion of one or more variant alleles of the human XPNPEP2 gene (e.g., wherein reference or wildtype allele is exemplified by a “G” at nucleotide 2172 of SEQ ID NO:893). Preferred portions are at least 10, preferably at least 20, preferably at least 40, preferably at least 100, contiguous polynucleotides and comprise one or more reference alleles at the nucleotide position(s) provided in Table I, II, or III.

The present invention relates to isolated nucleic acid molecules comprising, or alternatively, consisting of all or a portion of one or more variant alleles of the human XPNPEP2 gene (e.g., wherein alternate or variant allele is exemplified by an “A” at nucleotide 2172 of SEQ ID NO:893). Preferred portions are at least 10, preferably at least 20, preferably at least 40, preferably at least 100, contiguous polynucleotides and comprise one or more alternate (or variant) allele(s) at the nucleotide position(s) provided in Table I, II, or III. The invention further relates to isolated gene products, e.g., polypeptides and/or proteins, which are encoded by a nucleic acid molecule comprising all or a portion of at least one or more variant allele(s) of the gene.

In one embodiment, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with one or more reference allele(s) at the nucleotide position(s) provided in Table I, II, or III for XPNPEP2 gene (or diagnosing or aiding in the diagnosis of such a disorder) comprising the steps of obtaining a DNA sample from an individual to be assessed and determining the nucleotide present at said nucleotide position. The presence of the reference allele at said position indicates that the individual has a greater likelihood of having a disorder associated therewith than an individual having the alternate (variant) allele(s) at said position(s), or a greater likelihood of having more severe symptoms.

Conversely, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with the alternate (variant) allele(s) at the nucleotide position provided in Table I, II, or III for the XPNPEP2 gene (or diagnosing or aiding in the diagnosis of such a disorder) comprising the steps of obtaining a DNA sample from an individual to be assessed and determining the nucleotide present at said nucleotide position. The presence of the alternate (variant) allele(s) at said position(s) indicates that the individual has a greater likelihood of having a disorder associated therewith than an individual having the reference allele(s) at said position(s), or a greater likelihood of having more severe symptoms.

Representative disorders which may be detected, diagnosed, identified, treated, prevented, and/or ameliorated by the SNPs and methods of the present invention include, the following, non-limiting diseases and disorders: angioedema, susceptibility to acquiring an angioedema or an angioedema-like disorder upon the administration of a pharmaceutically acceptable amount of an ACE inhibitor and/or vasopeptidase inhibitor cardiovascular disease; angina pectoris, hypertension, heart failure, myocardial infarction, ventricular hypertrophy, cough associated with ACE inhibitors, cough associated with vasopeptidase inhibitors, vascular diseases, miscrovascular disease, vascular leak syndrome, aneurysm, stroke, embolism, thrombosis, endothelial dysfunction, coronary artery disease, arteriosclerosis, and/or atherosclerosis.

Additional disorders which may be detected, diagnosed, identified, treated, prevented, and/or ameliorated by the SNPs and methods of the present invention include, the following, non-limiting diseases and disorders: hypotensive reactions during blood transfusions (Transfusion. October 1999;39 (10):1084-8.), hypersensitivity reactions during hemodialysis (Peptides. 1999;20 (4):421-30.), sepsis, inflammatory arthritis, and enterocolitis (Clin Rev Allergy Immunol. 1998 Winter;16 (4):365-84.), enterocolitis (Gut. September 1998;43 (3):365-74.), chronic granulomatous intestinal and systemic inflammation (FASEB J. March 1998;12 (3):325-33.), peptidoglycan-induced arthritis (Arthritis Rheum. July 1997;40 (7): 1327-33.), arthritis (Proc Assoc Am Physicians. January 1997;109 (1):10-22.), intestinal inflammation (Dig Dis Sci. May 1996; 41 (5):912-20.), acute phase response of inflammation (Peptides. 1996;17 (7):1163-70.), asthma (J Appl Physiol 1995; 78: 1844-1852), chronic obstructive pulmonary disease (COPD), cough reflex, allergies, and/or neurogenic inflammation.

### Features of the Polypeptide Encoded by Gene No:7

The present invention relates to isolated nucleic acid molecules comprising, or alternatively, consisting of all or a portion of one or more variant alleles of the human GUCY1A2 gene (e.g., wherein reference or wildtype allele is exemplified by a “C” at nucleotide 2169; an “A” at nucleotide 825; and/or a “C” at nucleotide 822 of SEQ ID NO:895). Preferred portions are at least 10, preferably at least 20, preferably at least 40, preferably at least 100, contiguous polynucleotides and comprise one or more reference alleles at the nucleotide position(s) provided in Table I, II, or III.

The present invention relates to isolated nucleic acid molecules comprising, or alternatively, consisting of all or a portion of one or more variant alleles of the human GUCY1A2 gene (e.g., wherein alternate or variant allele is exemplified by a “T” at nucleotide 2169; a “G” at nucleotide 825; and/or a “T” at nucleotide 822 of SEQ ID NO:895). Preferred portions are at least 10, preferably at least 20, preferably at least 40, preferably at least 100, contiguous polynucleotides and comprise one or more alternate (or variant) allele(s) at the nucleotide position(s) provided in Table I, II, or III. The invention further relates to isolated gene products, e.g., polypeptides and/or proteins, which are encoded by a nucleic acid molecule comprising all or a portion of at least one or more variant allele(s) of the gene.

In one embodiment, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with one or more reference allele(s) at the nucleotide position(s) provided in Table I, II, or III for GUCY1A2 gene (or diagnosing or aiding in the diagnosis of such a disorder) comprising the steps of obtaining a DNA sample from an individual to be assessed and determining the nucleotide present at said nucleotide position. The presence of the reference allele at said position indicates that the individual has a greater likelihood of having a disorder associated therewith than an individual having the alternate (variant) allele(s) at said position(s), or a greater likelihood of having more severe symptoms.

Conversely, the invention relates to a method for predicting the likelihood that an individual will have a disorder, particularly angioedema or an angioedema-like disorder, or be susceptible to acquiring an angioedema or an angioedema-like disorder upon the administration of a pharmaceutically acceptable amount of an ACE inhibitor and/or vasopeptidase inhibitor, associated with the alternate (variant) allele(s) at the nucleotide position provided in Table I, II, or III for the GUCY1A2 gene (or diagnosing or aiding in the diagnosis of such a disorder) comprising the steps of obtaining a DNA sample from an individual to be assessed and determining the nucleotide present at said nucleotide position. The presence of the alternate (variant) allele(s) at said position(s) indicates that the individual has a greater likelihood of having a disorder associated therewith than an individual having the reference allele(s) at said position(s), or a greater likelihood of having more severe symptoms.

Representative disorders which may be detected, diagnosed, identified, treated, prevented, and/or ameliorated by the SNPs and methods of the present invention include, the following, non-limiting diseases and disorders: angioedema, susceptibility to acquiring an angioedema or an angioedema-like disorder upon the administration of a pharmaceutically acceptable amount of an ACE inhibitor and/or vasopeptidase inhibitor cardiovascular disease; angina pectoris, hypertension, heart failure, myocardial infarction, ventricular hypertrophy, cough associated with ACE inhibitors, cough associated with vasopeptidase inhibitors, vascular diseases, miscrovascular disease, vascular leak syndrome, aneurysm, stroke, embolism, thrombosis, endothelial dysfunction, coronary artery disease, arteriosclerosis, and/or atherosclerosis.

Additional disorders which may be detected, diagnosed, identified, treated, prevented, and/or ameliorated by the SNPs and methods of the present invention include, the following, non-limiting diseases and disorders: hypotensive reactions during blood transfusions (Transfusion. October 1999;39 (10):1084-8.), hypersensitivity reactions during hemodialysis (Peptides. 1999;20 (4):421-30.), sepsis, inflammatory arthritis, and enterocolitis (Clin Rev Allergy Immunol. 1998 Winter;16 (4):365-84.), enterocolitis (Gut. September 1998;43 (3):365-74.), chronic granulomatous intestinal and systemic inflammation (FASEB J. March 1998;12 (3):325-33.), peptidoglycan-induced arthritis (Arthritis Rheum. July 1997;40 (7):1327-33.), arthritis (Proc Assoc Am Physicians. January 1997;109 (1):10-22.), intestinal inflammation (Dig Dis Sci. May 1996; 41 (5):912-20.), acute phase response of inflammation (Peptides. 1996;17 (7):1163-70.), asthma (J Appl Physiol 1995; 78: 1844-1852), chronic obstructive pulmonary disease (COPD), cough reflex, allergies, and/or neurogenic inflammation.

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the protein, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, can be substantially purified using techniques described herein or otherwise known in the art, such as, for example, by the one-step method described in Smith and Johnson, Gene 67:31-40 (1988). Polypeptides of the invention also can be purified from natural, synthetic or recombinant sources using protocols described herein or otherwise known in the art, such as, for example, antibodies of the invention raised against the full-length form of the protein.

The present invention provides a polynucleotide comprising, or alternatively consisting of, the sequence identified as SEQ ID NO:X. The present invention also provides a polypeptide comprising, or alternatively consisting of, the sequence identified as SEQ ID NO:Y. The present invention also provides polynucleotides encoding a polypeptide comprising, or alternatively consisting of the polypeptide sequence of SEQ ID NO:Y.

Preferably, the present invention is directed to a polynucleotide comprising, or alternatively consisting of, the sequence identified as SEQ ID NO:X, that is less than, or equal to, a polynucleotide sequence that is 5 mega basepairs, 1 mega basepairs, 0.5 mega basepairs, 0.1 mega basepairs, 50,000 basepairs, 20,000 basepairs, or 10,000 basepairs in length.

The present invention encompasses polynucleotides with sequences complementary to those of the polynucleotides of the present invention disclosed herein. Such sequences may be complementary to the sequence disclosed as SEQ ID NO:X, and/or the nucleic acid sequence encoding the sequence disclosed as SEQ ID NO:Y.

The invention encompasses the application of PCR methodology to the polynucleotide sequences of the present invention, and/or the cDNA encoding the polypeptides of the present invention. PCR techniques for the amplification of nucleic acids are described in U.S. Pat. No. 4,683,195 and Saiki et al., Science, 239:487-491 (1988). PCR, for example, may include the following steps, of denaturation of template nucleic acid (if double-stranded), annealing of primer to target, and polymerization. The nucleic acid probed or used as a template in the amplification reaction may be genomic DNA, cDNA, RNA, or a PNA. PCR may be used to amplify specific sequences from genomic DNA, specific RNA sequence, and/or cDNA transcribed from mRNA. References for the general use of PCR techniques, including specific method parameters, include Mullis et al., Cold Spring Harbor Symp. Quant. Biol., 51:263, (1987), Ehrlich (ed), PCR Technology, Stockton Press, NY, 1989; Ehrlich et al., Science, 252:1643-1650, (1991); and “PCR Protocols, A Guide to Methods and Applications”, Eds., Innis et al., Academic Press, New York, (1990).

### Polynucleotide and Polypeptide Variants

The present invention also encompasses variants (e.g., allelic variants, orthologs, etc.) of the polynucleotide sequence disclosed herein in SEQ ID NO:X, the complementary strand thereto.

The present invention also encompasses variants of the polypeptide sequence, and/or fragments therein, disclosed in SEQ ID NO:Y, a polypeptide encoded by the polynucleotide sequence in SEQ ID NO:X.

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

Thus, one aspect of the invention provides an isolated nucleic acid molecule comprising, or alternatively consisting of, a polynucleotide having a nucleotide sequence selected from the group consisting of: (a) a nucleotide sequence encoding a related polypeptide of the present invention having an amino acid sequence as shown in the Sequence Listing and described in SEQ ID NO:X; (b) a nucleotide sequence encoding a mature related polypeptide of the present invention having the amino acid sequence as shown in the Sequence Listing and described in SEQ ID NO:X; (c) a nucleotide sequence encoding a biologically active fragment of a related polypeptide of the present invention having an amino acid sequence shown in the Sequence Listing and described in SEQ ID NO:X; (d) a nucleotide sequence encoding an antigenic fragment of a related polypeptide of the present invention having an amino acid sequence sown in the Sequence Listing and described in SEQ ID NO:X; (e) a nucleotide sequence encoding a related polypeptide of the present invention comprising the complete amino acid sequence encoded by a human cDNA plasmid contained in SEQ ID NO:X; (f) a nucleotide sequence encoding a mature related polypeptide of the present invention having an amino acid sequence encoded by a human cDNA plasmid contained in SEQ ID NO:X; (g) a nucleotide sequence encoding a biologically active fragment of a related polypeptide of the present invention having an amino acid sequence encoded by a human cDNA plasmid contained in SEQ ID NO:X; (h) a nucleotide sequence encoding an antigenic fragment of a related polypeptide of the present invention having an amino acid sequence encoded by a human cDNA plasmid contained in SEQ ID NO:X; (I) a nucleotide sequence complimentary to any of the nucleotide sequences in (a), (b), (c), (d), (e), (f), (g), or (h), above.

The present invention is also directed to polynucleotide sequences which comprise, or alternatively consist of, a polynucleotide sequence which is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, any of the nucleotide sequences in (a), (b), (c), (d), (e), (f), (g), or (h), above. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), (e), (f), (g), or (h), above. Polynucleotides which hybridize to the complement of these nucleic acid molecules under stringent hybridization conditions or alternatively, under lower stringency conditions, are also encompassed by the invention, as are polypeptides encoded by these polypeptides.

Another aspect of the invention provides an isolated nucleic acid molecule comprising, or alternatively, consisting of, a polynucleotide having a nucleotide sequence selected from the group consisting of: (a) a nucleotide sequence encoding a related polypeptide of the present invention having an amino acid sequence as shown in the Sequence Listing and described in Table I; (b) a nucleotide sequence encoding a mature related polypeptide of the present invention having the amino acid sequence as shown in the Sequence Listing and described in Table I; (c) a nucleotide sequence encoding a biologically active fragment of a related polypeptide of the present invention having an amino acid sequence as shown in the Sequence Listing and described in Table I; (d) a nucleotide sequence encoding an antigenic fragment of a related polypeptide of the present invention having an amino acid sequence as shown in the Sequence Listing and described in Table I; (e) a nucleotide sequence complimentary to any of the nucleotide sequences in (a), (b), (c), (d), or (e) above.

The present invention is also directed to nucleic acid molecules which comprise, or alternatively, consist of, a nucleotide sequence which is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, any of the nucleotide sequences in (a), (b), (c), (d), or (e) above.

The present invention encompasses polypeptide sequences which comprise, or alternatively consist of, an amino acid sequence which is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, the following non-limited examples, the polypeptide sequence identified as SEQ ID NO:Y, and/or polypeptide fragments of any of the polypeptides provided herein. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), or (e) above. Polynucleotides which hybridize to the complement of these nucleic acid molecules under stringent hybridization conditions or alternatively, under lower stringency conditions, are also encompassed by the invention, as are polypeptides encoded by these polypeptides.

The present invention is also directed to polypeptides which comprise, or alternatively consist of, an amino acid sequence which is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, the polypeptide sequence shown in SEQ ID NO:Y, a polypeptide sequence encoded by the nucleotide sequence in SEQ ID NO:X, a polypeptide sequence encoded by the cDNA provided in Table I, and/or polypeptide fragments of any of these polypeptides (e.g., those fragments described herein). Polynucleotides which hybridize to the complement of the nucleic acid molecules encoding these polypeptides under stringent hybridization conditions or alternatively, under lower stringency conditions, are also encompasses by the present invention, as are the polypeptides encoded by these polynucleotides.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding the polypeptide. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence referenced in Table I, the ORF (open reading frame), or any fragment specified as described herein.

As a practical matter, whether any particular nucleic acid molecule or polypeptide is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2 (22):4673-4680, (1994)), which is based on the algorithm of Higgins, D. G., et al., Computer Applications in the Biosciences (CABIOS), 8 (2):189-191, (1992). In a sequence alignment the query and subject sequences are both DNA sequences. An RNA sequence can be compared by converting U's to T's. The result of said global sequence alignment is in percent identity. Preferred parameters used in a CLUSTALW alignment of DNA sequences to calculate percent identify are: Matrix=BLOSUM, k-tuple=1, Number of Top Diagonals=5, Gap Penalty=3, Gap Open Penalty 10, Gap Extension Penalty=0, Scoring Method=Percent, Window Size=5 or the length of the subject nucleotide sequence, whichever is shorter.

If the subject sequence is shorter than the query sequence because of 5′ or 3′ deletions, not because of internal deletions, a manual correction must be made to the results. This is because the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score is what may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for instance, an amino acid sequence referenced in Table I or Table VI (SEQ ID NO:Y) can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2 (22):4673-4680, (1994)), which is based on the algorithm of Higgins, D. G., et al., Computer Applications in the Biosciences (CABIOS), 8 (2):189-191, (1992). In a sequence alignment the query and subject sequences are both DNA sequences. An RNA sequence can be compared by converting U's to T's. The result of said global sequence alignment is in percent identity. Preferred parameters used in a CLUSTALW amino acid alignment are: Matrix=BLOSUM, k-tuple=l, Number of Top Diagonals=5, Gap Penalty=3, Gap Open Penalty 10, Gap Extension Penalty=0, Scoring Method=Percent, Window Size=5 or the length of the subject nucleotide sequence, whichever is shorter.

If the subject sequence is shorter than the query sequence due to N- or C-terminal deletions, not because of internal deletions, a manual correction must be made to the results. This is because the CLUSTALW program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the CLUSTALW sequence alignment. This percentage is then subtracted from the percent identity, calculated by the above CLUSTALW program using the specified parameters, to arrive at a final percent identity score. This final percent identity score is what may be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons, e.g., to optimize codon expression for a particular host (change codons in the mRNA to those preferred by a bacterial host such as E. coli).

Naturally occurring variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level and are included in the present invention. Alternatively, non-naturally occurring variants may be produced by mutagenesis techniques or by direct synthesis.

Thus, the invention further includes polypeptide variants that show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include, but are not limited to, the following: (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as, for example, an IgG Fe fusion region peptide, or leader or secretory sequence, or a sequence facilitating purification. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

Moreover, the invention further includes polypeptide variants created through the application of molecular evolution (“DNA Shuffling”) methodology to the polynucleotide disclosed as SEQ ID NO:X, and/or the cDNA encoding the polypeptide disclosed as SEQ ID NO:Y. Such DNA Shuffling technology is known in the art and more particularly described elsewhere herein (e.g., WPC, Stemmer, PNAS, 91:10747, (1994)), and in the Examples provided herein).

A further embodiment of the invention relates to a polypeptide which comprises the amino acid sequence of the present invention having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a peptide or polypeptide to have an amino acid sequence which comprises the amino acid sequence of the present invention, which contains at least one, but not more than 10, 9, 8, 7, 6, 5, 4, 3, 2 or 1 amino acid substitutions. In specific embodiments, the number of additions, substitutions, and/or deletions in the amino acid sequence of the present invention or fragments thereof (e.g., the mature form and/or other fragments described herein), is 1-5, 5-10, 5-25, 5-50, 10-50 or 50-150, conservative amino acid substitutions are preferable.

### Polynucleotide and Polypeptide Fragments

The present invention is directed to polynucleotide fragments of the polynucleotides of the invention, in addition to polypeptides encoded therein by said polynucleotides and/or fragments.

In the present invention, a “polynucleotide fragment” refers to a short polynucleotide having a nucleic acid sequence which: is a portion of that shown in SEQ ID NO:X or the complementary strand thereto, or is a portion of a polynucleotide sequence encoding the polypeptide of SEQ ID NO:Y. The nucleotide fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A fragment “at least 20 nt in length,” for example, is intended to include 20 or more contiguous bases from the cDNA sequence shown in SEQ ID NO:X. In this context “about” includes the particularly recited value, a value larger or smaller by several (5, 4, 3, 2, or 1) nucleotides, at either terminus, or at both termini. These nucleotide fragments have uses that include, but are not limited to, as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:X, or the complementary strand thereto. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Also encompassed by the present invention are polynucleotides which hybridize to these nucleic acid molecules under stringent hybridization conditions or lower stringency conditions, as are the polypeptides encoded by these polynucleotides.

In the present invention, a “polypeptide fragment” refers to an amino acid sequence which is a portion of that contained in SEQ ID NO:Y. Protein (polypeptide) fragments may be “free-standing,” or comprised within a larger polypeptide of which the fragment forms a part or region, most preferably as a single continuous region. Representative examples of polypeptide fragments of the invention, include, for example, fragments comprising, or alternatively consisting of, from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges or values, and ranges or values larger or smaller by several (5, 4, 3, 2, or 1) amino acids, at either extreme or at both extremes. Polynucleotides encoding these polypeptides are also encompassed by the invention.

Preferred polypeptide fragments include the full-length protein. Further preferred polypeptide fragments include the full-length protein having a continuous series of deleted residues from the amino or the carboxy terminus, or both. For example, any number of amino acids, ranging from 1-60, can be deleted from the amino terminus of the full-length polypeptide. Similarly, any number of amino acids, ranging from 1-30, can be deleted from the carboxy terminus of the full-length protein. Furthermore, any combination of the above amino and carboxy terminus deletions are preferred. Similarly, polynucleotides encoding these polypeptide fragments are also preferred.

Also preferred are polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of SEQ ID NO:Y falling within conserved domains are specifically contemplated by the present invention. Moreover, polynucleotides encoding these domains are also contemplated.

Other preferred polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity. Polynucleotides encoding these polypeptide fragments are also encompassed by the invention.

In a preferred embodiment, the functional activity displayed by a polypeptide encoded by a polynucleotide fragment of the invention may be one or more biological activities typically associated with the full-length polypeptide of the invention. Illustrative of these biological activities includes the fragments ability to bind to at least one of the same antibodies which bind to the full-length protein, the fragments ability to interact with at lease one of the same proteins which bind to the full-length, the fragments ability to elicit at least one of the same immune responses as the full-length protein (i.e., to cause the immune system to create antibodies specific to the same epitope, etc.), the fragments ability to bind to at least one of the same polynucleotides as the full-length protein, the fragments ability to bind to a receptor of the full-length protein, the fragments ability to bind to a ligand of the full-length protein, and the fragments ability to multimerize with the full-length protein. However, the skilled artisan would appreciate that some fragments may have biological activities which are desirable and directly inapposite to the biological activity of the full-length protein. The functional activity of polypeptides of the invention, including fragments, variants, derivatives, and analogs thereof can be determined by numerous methods available to the skilled artisan, some of which are described elsewhere herein.

The present invention encompasses polypeptides comprising, or alternatively consisting of, an epitope of the polypeptide having an amino acid sequence of SEQ ID NO:Y, or encoded by a polynucleotide that hybridizes to the complement of the sequence of SEQ ID NO:X under stringent hybridization conditions or lower stringency hybridization conditions as defined supra. The present invention further encompasses polynucleotide sequences encoding an epitope of a polypeptide sequence of the invention (such as, for example, the sequence disclosed in SEQ ID NO:1), polynucleotide sequences of the complementary strand of a polynucleotide sequence encoding an epitope of the invention, and polynucleotide sequences which hybridize to the complementary strand under stringent hybridization conditions or lower stringency hybridization conditions defined supra.

The term “epitopes,” as used herein, refers to portions of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross- reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length, or longer. Additional non-exclusive preferred antigenic epitopes include the antigenic epitopes disclosed herein, as well as portions thereof Antigenic epitopes are useful, for example, to raise antibodies, including monoclonal antibodies, that specifically bind the epitope. Preferred antigenic epitopes include the antigenic epitopes disclosed herein, as well as any combination of two, three, four, five or more of these antigenic epitopes. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl- N-hydroxysuccinimide ester (MBS), while other peptides may be coupled to carriers using a more general linking agent such as glutaraldehyde. Animals such as rabbits, rats and mice are immunized with either free or carrier- coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and as discussed above, the polypeptides of the present invention comprising an immunogenic or antigenic epitope can be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+nitriloacetic acid-agarose column and histidine-tagged proteins can be selectively eluted with imidazole-containing buffers.

### Antibodies

Further polypeptides of the invention relate to antibodies and T-cell antigen receptors (TCR) which immunospecifically bind a polypeptide, polypeptide fragment, or variant of SEQ ID NO:Y, and/or an epitope, of the present invention (as determined by immunoassays well known in the art for assaying specific antibody-antigen binding). Antibodies of the invention include, but are not limited to, polyclonal, monoclonal, monovalent, bispecific, heteroconjugate, multispecific, human, humanized or chimeric antibodies, single chain antibodies, Fab fragments, F (ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Mab) is meant to include intact molecules, as well as, antibody fragments (such as, for example, Fab and F (ab′)2 fragments) which are capable of specifically binding to protein. Fab and F (ab′)2 fragments lack the Fc fragment of intact antibody, clear more rapidly from the circulation of the animal or plant, and may have less non-specific tissue binding than an intact antibody (Wahl et al., J. Nucl. Med.. 24:316-325 (1983)). Thus, these fragments are preferred, as well as the products of a FAB or other immunoglobulin expression library. Moreover, antibodies of the present invention include chimeric, single chain, and humanized antibodies.

Most preferably the antibodies are human antigen-binding antibody fragments of the present invention and include, but are not limited to, Fab, Fab′ and F (ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homologue of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologues of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5 X 10-2 M, 10-2 M, 5 X 10-3 M, 10-3 M, 5 X 10-4 M, 10-4 M, 5 X 10-5 M, 10-5 M, 5 X 10-6 M, 10-6M, 5 X 10-7 M, 107 M, 5 X 10-8 M, 10-8 M, 5 X 10-9 M, 10-9 M, 5 X 10-10 M, 10-10 M, 5 X 10-11 M, 10-11 M, 5 X 10-12 M, 10-12 M, 5 X 10-13 M, 10-13 M, 5 X 10-14 M, 10-14 M, 5 X 10-15 M, or 10-15 M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, or at least 50%.

Antibodies of the present invention may act as agonists or antagonists of the polypeptides of the present invention. Preferably the antibodies of the present invention are specific for a single nucleotide polymorphism of any one of the angioedema candidate gene polypeptides of the present invention. More preferred are antibodies that are capable of specifically distinquiching between the variant and reference forms of a polypeptide of the present invention. Such antibodies are primarily useful in a kit to identify variant or normal forms of a polypeptide, and hence determining whether a particular individual is at a higher or lower risk of being susceptible to angioedema or an angioedema-like disorder upon the administration of an ACE or vasopeptidase inhibitor.

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art.

The antibodies of the present invention may comprise polyclonal antibodies. Methods of preparing polyclonal antibodies are known to the skilled artisan (Harlow, et al., Antibodies: A Laboratory Manual, (Cold spring Harbor Laboratory Press, 2^{nd} ed. (1988), which is hereby incorporated herein by reference in its entirety). For example, a polypeptide of the invention can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art. For the purposes of the invention, “immunizing agent” may be defined as a polypeptide of the invention, including fragments, variants, and/or derivatives thereof, in addition to fusions with heterologous polypeptides and other forms of the polypeptides described herein.

Typically, the immunizing agent and/or adjuvant will be injected in the mammal by multiple subcutaneous or intraperitoneal injections, though they may also be given intramuscularly, and/or through IV). The immunizing agent may include polypeptides of the present invention or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivitizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate). The immunization protocol may be selected by one skilled in the art without undue experimentation.

The antibodies of the present invention may comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2^{nd} ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies includes, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include polypeptides of the present invention or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, California and the American Type Culture Collection, Manassas, Va. As inferred throughout the specification, human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the polypeptides of the present invention. Preferably, the binding specificity of monoclonal antibodies produced by the hybridoma cells is determined by immunoprecipitation or by an in vitro binding assay, such as radioimmunoassay (RIA) or enzyme-linked immunoadsorbant assay (ELISA). Such techniques are known in the art and within the skill of the artisan. The binding affinity of the monoclonal antibody can, for example, be determined by the Scatchard analysis of Munson and Pollart, Anal. Biochem., 107:220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

The skilled artisan would acknowledge that a variety of methods exist in the art for the production of monoclonal antibodies and thus, the invention is not limited to their sole production in hydridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hydridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fe region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples herein. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1domain of the heavy chain.

For example, the antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F (ab′)2 fragments can also be employed using methods known in the art such as those disclosed in PCT publication WO 92/22324; Mullinax et al., BioTechniques 12 (6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28 (4/5):489-498 (1991); Studnicka et al., Protein Engineering 7 (6):805-814 (1994); Roguska. et al., PNAS 91:969-973 (1994)), and chain shuffling (U.S. Pat. No. 5,565,332). Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., Nature, 321:522-525 (1986); Reichmann et al., Nature, 332:323-327 (1988); Verhoeyen et al., Science, 239:1534-1536 (1988), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature 332:323-329 (1988)1 and Presta, Curr. Op. Struct. Biol., 2:593-596 (1992).

Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of cole et al., and Boerder et al., are also available for the preparation of human monoclonal antibodies (cole et al., Monoclonal Antibodies and Cancer Therapy, Alan R. Riss, (1985); and Boerner et al., J. Immunol., 147 (1):86-95, (1991)).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and creation of an antibody repertoire. This approach is described, for example, in U.S. Pat. Nos. 5,545,807; 5,545,806; 5,569,825; 5,625,126; 5,633,425; 5,661,106, and in the following scientific publications: Marks et al., Biotechnol., 10:779-783 (1992); Lonberg et al., Nature 368:856-859 (1994); Fishwild et al., Nature Biotechnol., 14:845-51 (1996); Neuberger, Nature Biotechnol., 14:826 (1996); Lonberg and Huszer, Intern. Rev. Immunol., 13:65-93 (1995).

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7 (5):437-444; (1989) and Nissinoff, J. Immunol. 147 (8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

The antibodies of the present invention may be bispecific antibodies. Bispecific antibodies are monoclonal, preferably human or humanized, antibodies that have binding specificities for at least two different antigens. In the present invention, one of the binding specificities may be directed towards a polypeptide of the present invention, the other may be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transformed into a suitable host organism. For further details of generating bispecific antibodies see, for example Suresh et al., Meth. In Enzym., 121:210 (1986).

Heteroconjugate antibodies are also contemplated by the present invention. Heteroconjugate antibodies are composed of two covalently joined antibodies. Such antibodies have, for example, been proposed to target immune system cells to unwanted cells (U.S. Pat. No. 4,676,980), and for the treatment of HIV infection (WO 91/00360; WO 92/20373; and EP03089). It is contemplated that the antibodies may be prepared in vitro using known methods in synthetic protein chemistry, including those involving crosslinking agents. For example, immunotoxins may be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

### Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem . . . 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non- essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt- cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl . Acad. Sci. USA 78:1527 (1981)); gpt, which confers resistance to mycophenolic acid (Mulligan & Berg, Proc. NatI. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11 (5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452 (1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the present invention can form dimers through disulfide bonding between the Fe portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341 (1992) (said references incorporated by reference in their entireties).

As discussed, supra, the polypeptides corresponding to a polypeptide, polypeptide fragment, or a variant of SEQ ID NO:Y may be fused or conjugated to the above antibody portions to increase the in vivo half life of the polypeptides or for use in immunoassays using methods known in the art. Further, the polypeptides corresponding to SEQ ID NO:Y may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide- linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fe portions for the purpose of high-throughput screening assays to identify antagonists of h1L-5. (See, Bennett et al., J. Molecular Recognition 8:52-58 (1995); Johanson et al., J. Biol. Chem. 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl . Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 1251, 131I, 111In or 99Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

Uses for Antibodies directed against polypeptides of the invention The antibodies of the present invention have various utilities. For example, such antibodies may be used in diagnostic assays to detect the presence or quantification of a variant or reference form of a polypeptides of the invention in a sample. Such a diagnostic assay may be comprised of at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278 (2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), ppl47-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); Dafvid et al., Biochem., 13:1014 (1974); Pain et al., J. Immunol. Metho., 40:219 (1981); and Nygren, J. Histochem. And Cytochem., 30:407 (1982).

### Assays for Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

### Therapeutic/Prophylactic Administration and Compositions

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem.. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353- 365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Diagnosis and Imaging With Antibodies

Labeled antibodies, and derivatives and analogs thereof, which specifically bind to a variant or reference allele of a polypeptide of interest can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell . Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (112In), and technetium (99Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: (a) administering (for example, parenterally, subcutaneously, or intraperitoneally) to a subject an effective amount of a labeled molecule which specifically binds to the polypeptide of interest; (b) waiting for a time interval following the administering for permitting the labeled molecule to preferentially concentrate at sites in the subject where the polypeptide is expressed (and for unbound labeled molecule to be cleared to background level); (c) determining background level; and (d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

### Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or calorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

### Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carlsbad, Calif.). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In one embodiment, the yeast Pichia pastoris is used to express the polypeptide of the present invention in a eukaryotic system. Pichia pastoris is a methylotrophic yeast which can metabolize methanol as its sole carbon source. A main step in the methanol metabolization pathway is the oxidation of methanol to formaldehyde using O2. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O2. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al., Mol. Cell. Biol. 5:1111-21 (1985); Koutz, P. J, et al., Yeast 5:167-77 (1989); Tschopp, J. F., et al., Nucl. Acids Res. 15:3859-76 (1987). Thus, a heterologous coding sequence, such as, for example, a polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a polypeptide of the invention, as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PAO815, as one skilled in the art would readily appreciate, as long as the proposed expression construct provides appropriately located signals for transcription, translation, secretion (if desired), and the like, including an in-frame AUG, as required.

In another embodiment, high-level expression of a heterologous coding sequence, such as, for example, a polynucleotide of the present invention, may be achieved by cloning the heterologous polynucleotide of the invention into an expression vector such as, for example, pGAPZ or pGAPZalpha, and growing the yeast culture in the absence of methanol.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination, resulting in the formation of a new transcription unit (see, e.g., U.S. Pat. No. 5,641,670, issued Jun. 24, 1997; U.S. Pat. No. 5,733,761, issued Mar. 31, 1998; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

In addition, polypeptides of the invention can be chemically synthesized using techniques known in the art (e.g., see Creighton, 1983, Proteins: Structures and Molecular Principles, W. H. Freeman & Co., N.Y., and Hunkapiller et al., Nature, 310:105-111 (1984)). For example, a polypeptide corresponding to a fragment of a polypeptide sequence of the invention can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, omithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein, the addition of epitope tagged peptide fragments (e.g., FLAG, HA, GST, thioredoxin, maltose binding protein, etc.), attachment of affinity tags such as biotin and/or streptavidin, the covalent attachment of chemical moieties to the amino acid backbone, N- or C-terminal processing of the polypeptides ends (e.g., proteolytic processing), deletion of the N-terminal methionine residue, etc.

Also provided by the invention are chemically modified derivatives of the polypeptides of the invention which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The invention further encompasses chemical derivitization of the polypeptides of the present invention, preferably where the chemical is a hydrophilic polymer residue. Exemplary hydrophilic polymers, including derivatives, may be those that include polymers in which the repeating units contain one or more hydroxy groups (polyhydroxy polymers), including, for example, poly (vinyl alcohol); polymers in which the repeating units contain one or more amino groups (polyamine polymers), including, for example, peptides, polypeptides, proteins and lipoproteins, such as albumin and natural lipoproteins; polymers in which the repeating units contain one or more carboxy groups (polycarboxy polymers), including, for example, carboxymethylcellulose, alginic acid and salts thereof, such as sodium and calcium alginate, glycosaminoglycans and salts thereof, including salts of hyaluronic acid, phosphorylated and sulfonated derivatives of carbohydrates, genetic material, such as interleukin-2 and interferon, and phosphorothioate oligomers; and polymers in which the repeating units contain one or more saccharide moieties (polysaccharide polymers), including, for example, carbohydrates.

The molecular weight of the hydrophilic polymers may vary, and is generally about 50 to about 5,000,000, with polymers having a molecular weight of about 100 to about 50,000 being preferred. The polymers may be branched or unbranched. More preferred polymers have a molecular weight of about 150 to about 10,000, with molecular weights of 200 to about 8,000 being even more preferred.

For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

Additional preferred polymers which may be used to derivatize polypeptides of the invention, include, for example, poly (ethylene glycol) (PEG), poly (vinylpyrrolidine), polyoxomers, polysorbate and poly (vinyl alcohol), with PEG polymers being particularly preferred. Preferred among the PEG polymers are PEG polymers having a molecular weight of from about 100 to about 10,000. More preferably, the PEG polymers have a molecular weight of from about 200 to about 8,000, with PEG 2,000, PEG 5,000 and PEG 8,000, which have molecular weights of 2,000, 5,000 and 8,000, respectively, being even more preferred. Other suitable hydrophilic polymers, in addition to those exemplified above, will be readily apparent to one skilled in the art based on the present disclosure. Generally, the polymers used may include polymers that can be attached to the polypeptides of the invention via alkylation or acylation reactions.

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminus) available for derivatization in a particular protein. Under the appropriate reaction conditions, substantially selective derivatization of the protein at the N-terminus with a carbonyl group containing polymer is achieved.

As with the various polymers exemplified above, it is contemplated that the polymeric residues may contain functional groups in addition, for example, to those typically involved in linking the polymeric residues to the polypeptides of the present invention. Such functionalities include, for example, carboxyl, amine, hydroxy and thiol groups. These functional groups on the polymeric residues can be further reacted, if desired, with materials that are generally reactive with such functional groups and which can assist in targeting specific tissues in the body including, for example, diseased tissue. Exemplary materials which can be reacted with the additional functional groups include, for example, proteins, including antibodies, carbohydrates, peptides, glycopeptides, glycolipids, lectins, and nucleosides.

In addition to residues of hydrophilic polymers, the chemical used to derivatize the polypeptides of the present invention can be a saccharide residue. Exemplary saccharides which can be derived include, for example, monosaccharides or sugar alcohols, such as erythrose, threose, ribose, arabinose, xylose, lyxose, fructose, sorbitol, mannitol and sedoheptulose, with preferred monosaccharides being fructose, mannose, xylose, arabinose, mannitol and sorbitol; and disaccharides, such as lactose, sucrose, maltose and cellobiose. Other saccharides include, for example, inositol and ganglioside head groups. Other suitable saccharides, in addition to those exemplified above, will be readily apparent to one skilled in the art based on the present disclosure. Generally, saccharides which may be used for derivitization include saccharides that can be attached to the polypeptides of the invention via alkylation or acylation reactions.

Moreover, the invention also encompasses derivitization of the polypeptides of the present invention, for example, with lipids (including cationic, anionic, polymerized, charged, synthetic, saturated, unsaturated, and any combination of the above, etc.). stabilizing agents.

The invention encompasses derivitization of the polypeptides of the present invention, for example, with compounds that may serve a stabilizing function (e.g., to increase the polypeptides half-life in solution, to make the polypeptides more water soluble, to increase the polypeptides hydrophilic or hydrophobic character, etc.). Polymers useful as stabilizing materials may be of natural, semi-synthetic (modified natural) or synthetic origin. Exemplary natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, glucose, polyglucose, polydextrose, pustulan, chitin, agarose, keratin, chondroitin, dermatan, hyaluronic acid, alginic acid, xanthin gum, starch and various other natural homopolymer or heteropolymers, such as those containing one or more of the following aldoses, ketoses, acids or amines: erythose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof Accordingly, suitable polymers include, for example, proteins, such as albumin, polyalginates, and polylactide-coglycolide polymers. Exemplary semi-synthetic polymers include carboxymethylcellulose, hydroxymethylcellulose, hydroxypropylmethylcellulose, methylcellulose, and methoxycellulose. Exemplary synthetic polymers include polyphosphazenes, hydroxyapatites, fluoroapatite polymers, polyethylenes (such as, for example, polyethylene glycol (including for example, the class of compounds referred to as Pluronics.RTM., commercially available from BASF, Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof. Methods for the preparation of derivatized polypeptides of the invention which employ polymers as stabilizing compounds will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

Moreover, the invention encompasses additional modifications of the polypeptides of the present invention. Such additional modifications are known in the art, and are specifically provided, in addition to methods of derivitization, etc., in U.S. Pat. No. 6,028,066, which is hereby incorporated in its entirety herein.

The polypeptides of the invention may be in monomers or multimers (i.e., dimers, trimers, tetramers and higher multimers). Accordingly, the present invention relates to monomers and multimers of the polypeptides of the invention, their preparation, and compositions (preferably, Therapeutics) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only polypeptides corresponding to the amino acid sequence of SEQ ID NO:Y (including fragments, variants, splice variants, and fusion proteins, corresponding to these polypeptides as described herein). These homomers may contain polypeptides having identical or different amino acid sequences. In a specific embodiment, a homomer of the invention is a multimer containing only polypeptides having an identical amino acid sequence. In another specific embodiment, a homomer of the invention is a multimer containing polypeptides having different amino acid sequences. In specific embodiments, the multimer of the invention is a homodimer (e.g., containing polypeptides having identical or different amino acid sequences) or a homotrimer (e.g., containing polypeptides having identical and/or different amino acid sequences). In additional embodiments, the homomeric multimer of the invention is at least a homodimer, at least a homotrimer, or at least a homotetramer.

As used herein, the term heteromer refers to a multimer containing one or more heterologous polypeptides (i.e., polypeptides of different proteins) in addition to the polypeptides of the invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the polypeptides of the invention. Such covalent associations may involve one or more amino acid residues contained in the polypeptide sequence (e.g., that recited in the Sequence Listing). In one instance, the covalent associations are cross-linking between cysteine residues located within the polypeptide sequences which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a fusion protein of the invention.

In one example, covalent associations are between the heterologous sequence contained in a fusion protein of the invention (see, e.g., U.S. Pat. No. 5,478,925). In a specific example, the covalent associations are between the heterologous sequence contained in an Fc fusion protein of the invention (as described herein). In another specific example, covalent associations of fusion proteins of the invention are between heterologous polypeptide sequence from another protein that is capable of forming covalently associated multimers, such as for example, osteoprotegerin (see, e.g., International Publication NO: WO 98/49305, the contents of which are herein incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Another method for preparing multimer polypeptides of the invention involves use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Trimeric polypeptides of the invention may offer the advantage of enhanced biological activity. Preferred leucine zipper moieties and isoleucine moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. Other peptides derived from naturally occurring trimeric proteins may be employed in preparing trimeric polypeptides of the invention.

In another example, proteins of the invention are associated by interactions between Flag® polypeptide sequence contained in fusion proteins of the invention containing Flag® polypeptide sequence. In a further embodiment, associations proteins of the invention are associated by interactions between heterologous polypeptide sequence contained in Flag® fusion proteins of the invention and anti-Flag® antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hydrophobic or signal peptide) and which can be incorporated by membrane reconstitution techniques into liposomes (see, e.g., U.S. Pat. No. 5,478,925, which is herein incorporated by reference in its entirety).

In addition, the polynucleotide insert of the present invention could be operatively linked to “artificial” or chimeric promoters and transcription factors. Specifically, the artificial promoter could comprise, or alternatively consist, of any combination of cis-acting DNA sequence elements that are recognized by trans-acting transcription factors. Preferably, the cis acting DNA sequence elements and trans-acting transcription factors are operable in mammals. Further, the trans-acting transcription factors of such “artificial” promoters could also be “artificial” or chimeric in design themselves and could act as activators or repressors to said “artificial” promoter.

### Methods of Use of the Allelic Polynucleotides and Polypeptides of the Present Invention

The determination of the polymorphic form(s) present in an individual at one or more polymorphic sites defined herein can be used in a number of methods.

In preferred embodiments, the polynucleotides and polypeptides of the present invention, including allelic and variant forms thereof, have uses which include, but are not limited to diagnosing individuals to identify whether a given individual has increased susceptibility or risk for angioedema using the genotype assays of the present invention, and diagnosing individuals to identify whether a given individual, upon administration of an ACE inhibitor or vasopeptidase inhibitors, has increased susceptibility or risk for angioedema using the genotype assays of the present invention.

In another embodiment, the polynucleotides and polypeptides of the present invention, including allelic and variant forms thereof, either alone, or in combination with other polymorphic polynucleotides (haplotypes) are useful as genetic markers.

In preferred embodiments, the polynucleotides and polypeptides of the present invention, including allelic and variant forms thereof, have uses which include, but are not limited to diagnosing individuals to identify whether a given individual has increased susceptibility or risk for other conditions such as hypertension, congestive heart failure, and inflammatory bowel disease using the genotype assays of the present invention, and diagnosing individuals to identify whether a given individual, upon administration of a ACE inhibitors, vasopeptidase inhibitors, and/or any other cardiovascular drug known in the art or described herein, has increased susceptibility or risk for angioedema using the genotype assays of the present invention.

In preferred embodiments, the polynucleotides and polypeptides of the present invention, including allelic and variant forms thereof, have uses which include, but are not limited to diagnosing individuals to identify whether a given individual has increased susceptibility or risk for additional conditions, which include, the following, non-limiting examples: angioedema, cardiovascular diseases, angina pectoris, hypertension, heart failure, myocardial infarction, ventricular hypertrophy, cough associated with ACE inhibitors, cough associated with vasopeptidase inhibitors, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the polynucleotides and polypeptides of the present invention, including allelic and variant forms thereof, have uses which include, but are not limited to diagnosing individuals to identify whether a given individual has increased susceptibility or risk for additional conditions, which include, the following, non-limiting examples: hypotensive reactions during blood transfusions (Transfusion. October 1999;39 (10):1084-8.), hypersensitivity reactions during hemodialysis (Peptides. 1999;20 (4):421-30.), sepsis, inflammatory arthritis, and enterocolitis (Clin Rev Allergy Immunol. 1998 Winter;16 (4):365-84.), enterocolitis (Gut. September 1998;43 (3):365-74.), chronic granulomatous intestinal and systemic inflammation (FASEB J. March 1998;12 (3):325-33.), peptidoglycan-induced arthritis (Arthritis Rheum. July 1997; 40 (7):1327-33.), arthritis (Proc Assoc Am Physicians. January 1997;109 (1):10-22.), intestinal inflammation (Dig Dis Sci. May 1996;41 (5):912-20.), acute phase response of inflammation (Peptides. 1996;17 (7):1163-70.), asthma (J Appl Physiol 1995; 78: 1844-1852), chronic obstructive pulmonary disease (COPD), cough reflex, allergies, and/or neurogenic inflammation.

The polynucleotides and polypeptides of the present invention, including allelic and/or variant forms thereof, are useful for creating recombinant vectors and hosts cells for the expression of variant forms of the polypeptides of the present invention.

The polynucleotides and polypeptides of the present invention, including allelic and/or variant forms thereof, are useful for creating antagonists directed against these polynucleotides and polypeptides, particularly antibody antagonists, for diagnostic, and/or therapeutic applications.

Additionally, the polynucleotides and polypeptides of the present invention, including allelic and/or variant forms thereof, are useful for creating additional antagonists directed against these polynucleotides and polypeptides, which include, but are not limited to the design of antisense RNA, ribozymes, PNAs, recombinant zinc finger proteins (Wolfe, S A., Ramm, E I., Pabo, C O, Structure, Fold, Des., 8 (7):739-50, (2000); Kang, J S., Kim, J S, J. Biol, Chem., 275 (12):8742-8, (2000); Wang, B S., Pabo, C O, Proc, Natl, Acad, Sci, U, S, A., 96 (17):9568-73, (1999); McColl, D J., Honchell, C D., Frankel, A D, Proc, Natl, Acad, Sci, U, S, A., 96 (17):9521-6, (1999); Segal, D J., Dreier, B., Beerli, R R., Barbas, CF-3rd, Proc, Natl, Acad, Sci, U, S, A., 96 (6):2758-63, (1999); Wolfe, S A., Greisman, H A., Ramm, E I., Pabo, C O, J. Mol, Biol., 285 (5):1917-34, (1999); Pomerantz, J L., Wolfe, S A., Pabo, C O, Biochemistry., 37 (4):965-70, (1998); Leon, O., Roth, M., Biol. Res. 33 (1):21-30 (2000); Berg, J M., Godwin, H A, Ann. Rev. Biophys. Biomol. Struct., 26:357-71 (1997)), in addition to other types of antagonists which are either described elsewhere herein, or known in the art.

The polynucleotides and polypeptides of the present invention, including allelic and/or variant forms thereof, are useful for creating small molecule antagonists directed against the variant forms of these polynucleotides and polypeptides, preferably wherein such small molecules are useful as therapeutic and/or pharmaceutical compounds for the treatment, detection, prognosis, and/or prevention of the following, nonlimiting diseases and/or disorders, cardiovascular diseases, inflammatory diseases, angioedema, hypertension, and congestive heart failure.

The polynucleotides and polypeptides of the present invention, including allelic and/or variant forms thereof, are useful for the treatment of angioedema, hypertension, and congestive heart failure, in addition to other diseases and/or conditions referenced elsewhere herein, through the application of gene therapy based regimens.

Additional uses of the polynucleotides and polypeptides of the present invention are provided herein.

### Forensics

Determination of which polymorphic forms occupy a set of polymorphic sites in an individual identifies a set of polymorphic forms that distinguishes the individual. See generally National Research Council, The Evaluation of Forensic DNA Evidence (Eds. Pollard et al., National Academy Press, DC, 1996). The more sites that are analyzed, the lower the probability that the set of polymorphic forms in one individual is the same as that in an unrelated individual. Preferably, if multiple sites are analyzed, the sites are unlinked. Thus, polymorphisms of the invention are often used in conjunction with polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental eITor) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

(ID) is the probability that two random individuals have the same polymorphic or allelic form at a given polymorphic site. In biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote: p (AA)=x²

Homozygote: p(BB)=y²=(1−x)²

Single Heterozygote: p(AB)=p(BA)=xy=x(1−x)

Both Heterozygotes: p(AB+BA)=2xy=2x(1−x)

The probability of identity at one locus (i.e., the probability that two individuals, picked at random from a population will have identical polymorphic forms at a given locus) is given by the equation:

p(ID)=(x²)²+(2xy)²+(y²)²

These calculations can be extended for any number of polymorphic forms at a given locus. For example, the probability of identity p (m) for a 3-allele system where the alleles have the frequencies in the population of x, y and z, respectively, is equal to the sum of the squares of the genotype frequencies:

p(ID)=x⁴+(2xy)²+(2yz)²+(2xz)²+z⁴+y⁴

In a locus of n alleles, the appropriate binomial expansion is used to calculate p (ID) and p (exc).

The cumulative probability of identity (cum p (ID)) for each of multiple unlinked loci is determined by multiplying the probabilities provided by each locus.

cum p(ID)=p(ID)p(ID2)p(ID3) . . . p(IDn)

The cumulative probability of non-identity for n loci (i.e. the probability that two random individuals will be different at 1 or more loci) is given by the equation:

cum p(nonID)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

### Paternity Testing

The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child.

If the set of polymorphisms in the child attributable to the father does not match the set of polymorphisms of the putative father, it can be concluded, barring experimental error, that the putative father is not the real father.

If the set of polymorphisms in the child attributable to the father does match the set of polymorphisms of the putative father, a statistical calculation can be performed to determine the probability of coincidental match.

The probability of parentage exclusion (representing the probability that a random male will have a polymorphic form at a given polymorphic site that makes him incompatible as the father) is given by the equation (see WQ 95/12607):

p(exc)=xy(1−xy)

where x and y are the population frequencies of alleles A and B of a biallelic polymorphic site.

(At a triallelic site p(exc)=xy(1−xy)+yz(1−yz)+xz(1−xz)+3xyz(1−xyz))),

where x, y and z and the respective population frequencies of alleles A, B and C).

The probability of non-exclusion is

p(non-exc)=1−p(exc)

The cumulative probability of non-exclusion (representing the value obtained when n loci are used) is thus:

cum p(non-exc)=p(non-exc1)p(non-exc2)p(non-exc3) . . . p(non-excn)

The cumulative probability of exclusion for n loci (representing the probability that a random male will be excluded)

cum p(exc)=1−cum p(non-exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

### Correlation of Polymorphisms with Phenotypic Traits

The polymorphisms of the invention may contribute to the phenotype of an organism in different ways. Some polymorphisms occur within a protein coding sequence and contribute to phenotype by affecting protein structure. The effect may be neutral, beneficial or detrimental, or both beneficial and detrimental, depending on the circumstances. For example, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation. A single polymorphism may affect more than one phenotypic trait. Likewise, a single phenotypic trait may be affected by polymorphisms in different genes. Further, some polymorphisms predispose an individual to a distinct mutation that is causally related to a certain phenotype.

Phenotypic traits include diseases that have known but hitherto unmapped genetic components (e.g., agammaglobulimenia, diabetes insipidus, Lesch-Nyhan syndrome, muscular dystrophy, Wiskott-Aldrich syndrome, Fabry's disease, familial hypercholesterolemia, polycystic kidney disease, hereditary spherocytosis, von Willebrand's disease, tuberous sclerosis, hereditary hemorrhagic telangiectasia, familial colonic polyposis, Ehlers-Danlos syndrome, osteogenesis imperfecta, and acute intermittent porphyria). Phenotypic traits also include symptoms of, or susceptibility to, multifactorial diseases of which a component is or may be genetic, such as autoimmune diseases, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, obesity), strength, speed, endurance, fertility, and susceptibility or receptivity to particular drugs or therapeutic treatments.

The correlation of one or more polymorphisms with phenotypic traits can be facilitated by knowledge of the gene product of the wild type (reference) gene. The genes in which SNPs of the present invention have been identified are genes which have been previously sequenced and characterized in one of their allelic forms. Thus, the SNPs of the invention can be used to identify correlations between one or another allelic form of the gene with a disorder with which the gene is associated, thereby identifying causative or predictive allelic forms of the gene.

Correlation is performed for a population of individuals who have been tested for the presence or absence of a phenotypic trait of interest and for polymorphic markers sets. To perform such analysis, the presence or absence of a set of polymorphisms (i.e. a polymorphic set) is determined for a set of the individuals, some of whom exhibit a particular trait, and some of which exhibit lack of the trait. The alleles of each polymorphism of the set are then reviewed to determine whether the presence or absence of a particular allele is associated with the trait of interest. Correlation can be performed by standard statistical methods such as a 1C-squared test and statistically significant correlations between polymorphic form(s) and phenotypic characteristics are noted. For example, it might be found that the presence of allele A1 at polymorphism A correlates with heart disease. As a further example, it might be found that the combined presence of allele A1 at polymorphism A and allele B1 at polymorphism B correlates with increased milk production of a farm animal.

Such correlations can be exploited in several ways. In the case of a strong correlation between a set of one or more polymorphic forms and a disease for which treatment is available, detection of the polymorphic form set in a human or animal patient may justify immediate administration of treatment, or at least the institution of regular monitoring of the patient. Detection of a polymorphic form correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. Identification of a polymorphic set in a patient correlated with enhanced receptiveness to one of several treatment regimes for a disease indicates that this treatment regime should be followed.

For animals and plants, correlations between characteristics and phenotype are useful for breeding for desired characteristics. For example, Beitz et al, U.S. Pat. No. 5,292,639 discuss use of bovine mitochondrial polymorphisms in a breeding program to improve milk production in cows. To evaluate the effect of mtDNA D-loop sequence polymorphism on milk production, each cow was assigned a value of 1 if variant or 0 if wildtype with respect to a prototypical mitochondrial DNA sequence at each of 10 locations considered. Each production trait was analyzed individually with the following animal model:

Yᵢⱼₖₚₙ=υ+YSᵢ+Pⱼ+Xₖ+β₁+. . . β₁₇+PEₙ+aₙ+eₚ

where Yᵢⱼₖₚₙ is the milk, fat, fat percentage, SNF , SNF percentage, energy concentration, or lactation energy record; υ is an overall mean; YSᵢ is the effect common to all cows calving in year-season; Xₖ is the effect common to cows in either the high or average selection line; β₁ to β₁₇ are the binomial regressions of production record on mtDNA D-loop sequence polymorphisms; PEₙ is permanent environmental effect common to all records of cow n; aₙ is effect of animal n and is composed of the additive genetic contribution of sire and dam breeding values and a Mendelian sampling effect; and eₚ is a random residual. It was found that eleven of seventeen polymorphisms tested influenced at least one production trait. Bovines having the best polymorphic forms for milk production at these eleven loci are used as parents for breeding the next generation of the herd.

### Genetic Mapping of Phenotypic Traits

The previous section concerns identifying correlations between phenotypic traits and polymorphisms that directly or indirectly contribute to those traits. The present section describes identification of a physical linkage between a genetic locus associated with a trait of interest and polymorphic markers that are not associated with the trait, but are in physical proximity with the genetic locus responsible for the trait and cosegregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83:7353-7357 (1986); Lander et al., Proc. Natl. Acad. Sci. (USA) 84:2363-2367 (1987); Donis-Keller et al., Cell 51:319-337 (1987); Lander et al., Genetics 121:185-199 (1989)). Genese localized by linkage can be cloned by a process known as directional cloning. See Winwright, Med. J. Australia 159:170-174 (1993); Collins, Nature Genetics 1:3-6 (1992).

Linkage studies are typically performed on members of a family. Available numbers of the family are characterized for the presence or absence of a phenotypic trait and for a set of polymorphic markers. The distribution of polymorphic markers in an informative meiosis is then analyzed to determine which polymorphic markers cosegregate with a phenotypic trait. See, e.g., Kerem et al., Science 245:1073-1080 (1989); Monaco et al., Nature 316:842 (1985); Yamoka et al., Neurology 40:222-226 (1990); Rossiter et al., FASEB Journal, 5:21-27 (1991).

Linkage is analyzed by calculation of LOD (log of the odds) values. A LOS value is the realtive likelihood of obtaining observed segregation data for a marker and a genetic locus when the two are located at a recombination fraction θ, versus the situation in which the two are not linked, and thus segregating independently (Thompson & Thompson, Genetics in Medicine (5^{th} ed, W. B. Saunders Company, Philadelphia, 1991); Strachan, “Mapping the human genome” in The Human Gneome (BIOS Scientic Publishers Ltd, Oxford), Chapter 4). A series of likelihoos ratios are calculated at various recombination fractions (0), ranging from θ=0.0 (coincident loci) to θ=0.50 (unlinked). Thus, the likelihoos ata given value of θ is: probability of data if loci linked at θ to probability of data if loci are unlinked. The computed likelihoods are usually expressed as the log10 of this ratio (i.e., a LOD score). For example, a LOD score of 3 indicates 1000:1 odds against an apparent obsered linkage being a coincidence. The use of logarithms allows data collected from different familites to be combined by simple algorithm. Computer programs are available for the calculation of LOD scores for differing values of θ (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., lvlathematical tables for research workers in human genetics (Churchill, London, 1961); Smith, Ann. Hum. Genet. 32,127-150 (1968). The value of θ at which the lod score is the highest is considered to be the best estimate of the recombination fraction. Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

### Modified Polypeptides and Gene Sequences

The invention further provides variant forms of nucleic acids and corresponding proteins. The nucleic acids comprise one of the sequences described in Table I, or the polynucleotides encoding the polypeptides described in Table VIII, in which the polymorphic position is occupied by one of the alternative bases for that position. Some nucleic acids encode full-length variant forms of proteins. Variant genes can be expressed in an expression vector in which a variant gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g. , mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like. As used herein, “gene product” includes mRNA, peptide and protein products.

The protein may be isolated by conventional means of protein biochemistry and purification to obtain a substantially pure product, i.e., 80,95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

The invention further provides transgenic nonhuman animals capable of expressing an exogenous variant gene and/or having one or both alleles of an endogenous variant gene inactivated. Expression of an exogenous variant gene is usually achieved by operably linking the gene to a promoter and optionally an enhancer, and microinjecting the construct into a zygote. See Hogan et al., “Manipulating the Mouse Embryo, A Laboratory Manual,” Cold Spring Harbor Laboratory Inactivation of endogenous variant genes can be achieved by forming a trans gene in which a cloned variant gene is inactivated by insertion of a positive selection marker. See Capecchi, Science 244, 1288-1292 (1989). The trans gene is then introduced into an embryonic stem cell, where it undergoes homologous recombination with an endogenous variant gene. Mice and other rodents are preferred animals. Such animals provide useful drug screening systems.

In addition to substantially full-length polypeptides expressed by variant genes, the present invention includes biologically active fragments of the polypeptides, or analogs thereof, including organic molecules which simulate the interactions of the peptides. Biologically active fragments include any portion of the full-length polypeptide which confers a biological function on the variant gene product, including ligand binding, and antibody binding. Ligand binding includes binding by nucleic acids, proteins or polypeptides, small biologically active molecules, or large cellular structures.

Polyclonal and/or monoclonal antibodies that specifically bind to variant gene products but not to corresponding prototypical gene products are also provided. Antibodies can be made by injecting mice or other animals with the variant gene product or synthetic peptide fragments thereof. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

### Haplotype Based Genetic Analysis

The invention further provides methods of applying the polynucleotides and polypeptides of the present invention to the elucidation of haplotypes. Such haplotypes may be associated with any one or more of the disease conditions referenced elsewhere herein. A “haplotype” is defined as the pattern of a set of alleles of single nucleotide polymorphisms along a chromosome. For example, consider the case of three single nucleotide polymorphisms (SNP1, SNP2, and SNP3) in one chromosome region, of which SNP1 is an A/G polymorphism, SNP2 is a G/C polymorphism, and SNP3 is an A/C polymorphism. A and G are the alleles for the first, G and C for the second and A and C for the third SNP. Given two alleles for each SNP, there are three possible genotypes for individuals at each SNP. For example, for the first SNP, A/A, A/G and G/G are the possible genotypes for individuals. When an individual has a genotype for a SNP in which the alleles are not the same, for example A/G for the first SNP, then the individual is a heterozygote. When an individual has an A/G genotype at SNP1, G/C genotype at SNP2, and A/C genotype at SNP3 (FIG. 50), there are four possible combinations of haplotypes (A, B, C, and D) for this individual. The set of SNP genotypes of this individual alone would not provide sufficient information to resolve which combination of haplotypes this individual possesses. However, when this individual's parents' genotypes are available, haplotypes could then be assigned unambiguously. For example, if one parent had an A/A genotype at SNP1, a G/C genotype at SNP2, and an A/A genotype at SNP3, and the other parent had an A/G genotype at SNP1, C/C genotype at SNP2, and C/C genotype at SNP3, while the child was a heterozygote at all three SNPs (FIG. 51), there is only one possible haplotype combination, assuming there was no crossing over in this region during meiosis.

When the genotype information of relatives is not available, haplotype assignment can be done using the long range-PCR method (Clark, A. G. Mol Biol Evol 7 (2): 111-22 (1990); Clark, A. G., K. M. Weiss, et al.. Am J Hum Genet 63 (2): 595-612 (1998); Fullerton, S. M., A. G. Clark, et al., Am J Hum. Genet 67 (4): 881-900 (2000); Templeton, A. R., A. G. Clark, et al., Am J Hum Genet 66 (1): 69-83 (2000)). When the genotyping result of the SNPs of interest are available from general population samples, the most likely haplotypes can also be assigned using statistical methods (Excoffier, L. and M. Slatkin. Mol Biol Evol 12 (5): 921-7 (1995); Fallin, D. and N. J. Schork, Am J Hum Genet 67 (4): 947-59 (2000); Long, J. C., R. C. Williams, et al., Am J Hum Genet 56 (3): 799-810 (1995)).

Once an individual's haplotype in a certain chromosome region (i.e., locus) has been determined, it can be used as a tool for genetic association studies using different methods, which include, for example, haplotype relative risk analysis (Knapp, M., S. A. Seuchter, et al., Am J Hum Genet 52 (6): 1085-93 (1993); Li, T., M. Arranz, et al., Schizophr Res 32 (2): 87-92 (1998); Matise, T. C., Genet Epidemiol 12 (6): 641-5 (1995); Ott, J., Genet Epidemiol 6 (1): 127-30 (1989); Terwilliger, J and J. Ott, Hum Hered 42 (6): 337-46 (1992)). Haplotype based genetic analysis, using a combination of SNPs, provides increased detection sensitivity, and hence statistical significance, for genetic associations of diseases, as compared to analyses using individual SNPs as markers. Multiple SNPs present in a single gene or a continuous chromosomal region are useful for such haplotype-based analyses.

### Kits

The invention further provides kits comprising at least one agent for identifying which alleleic form of the SNPs identified herein is present in a sample. For example, suitable kits can comprise at least one antibody specific for a particular protein or peptide encoded by one alleleic form of the gene, or allele-specific oligonucleotide as described herein. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 1, 10, 100 or all of the polymorphisms shown in Table I. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

### Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents, based on actual sequence data (repeat polymorphisms), are presently available. Each polynucleotide of the present invention can be used as a chromosome marker.

Briefly, sequences can be mapped to chromosomes by preparing PCR primers (preferably 15-25 bp) from the sequences shown in SEQ ID NO:X. Primers can be selected using computer analysis so that primers do not span more than one predicted exon in the genomic DNA. These primers are then used for PCR screening of somatic cell hybrids containing individual human chromosomes. Only those hybrids containing the human gene corresponding to the SEQ ID NO:X will yield an amplified fragment.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. Disease mapping data are known in the art. Assuming 1 megabase mapping resolution and one gene per 20 kb, a cDNA precisely localized to a chromosomal region associated with the disease could be one of 50-500 potential causative genes.

Thus, once coinheritance is established, differences in the polynucleotide and the corresponding gene between affected and unaffected organisms can be examined. First, visible structural alterations in the chromosomes, such as deletions or translocations, are examined in chromosome spreads or by PCR. If no structural alterations exist, the presence of point mutations are ascertained. Mutations observed in some or all affected organisms, but not in normal organisms, indicates that the mutation may cause the disease. However, complete sequencing of the polypeptide and the corresponding gene from several normal organisms is required to distinguish the mutation from a polymorphism. If a new polymorphism is identified, this polymorphic polypeptide can be used for further linkage analysis.

Furthermore, increased or decreased expression of the gene in affected organisms as compared to unaffected organisms can be assessed using polynucleotides of the present invention. Any of these alterations (altered expression, chromosomal rearrangement, or mutation) can be used as a diagnostic or prognostic marker.

Thus, the invention also provides a diagnostic method useful during diagnosis of a disorder, involving measuring the expression level of polynucleotides of the present invention in cells or body fluid from an organism and comparing the measured gene expression level with a standard level of polynucleotide expression level, whereby an increase or decrease in the gene expression level compared to the standard is indicative of a disorder.

By “measuring the expression level of a polynucleotide of the present invention” is intended qualitatively or quantitatively measuring or estimating the level of the polypeptide of the present invention or the level of the mRNA encoding the polypeptide in a first biological sample either directly (e.g., by determining or estimating absolute protein level or mRNA level) or relatively (e.g., by comparing to the polypeptide level or mRNA level in a second biological sample). Preferably, the polypeptide level or mRNA level in the first biological sample is measured or estimated and compared to a standard polypeptide level or mRNA level, the standard being taken from a second biological sample obtained from an individual not having the disorder or being determined by averaging levels from a population of organisms not having a disorder. As will be appreciated in the art, once a standard polypeptide level or mRNA level is known, it can be used repeatedly as a standard for comparison.

By “biological sample” is intended any biological sample obtained from an organism, body fluids, cell line, tissue culture, or other source which contains the polypeptide of the present invention or mRNA. As indicated, biological samples include body fluids (such as the following non-limiting examples, sputum, amniotic fluid, urine, saliva, breast milk, secretions, interstitial fluid, blood, serum, spinal fluid, etc.) which contain the polypeptide of the present invention, and other tissue sources found to express the polypeptide of the present invention. Methods for obtaining tissue biopsies and body fluids from organisms are well known in the art. Where the biological sample is to include mRNA, a tissue biopsy is the preferred source.

The method(s) provided above may Preferably be applied in a diagnostic method and/or kits in which polynucleotides and/or polypeptides are attached to a solid support. In one exemplary method, the support may be a “gene chip” or a “biological chip” as described in U.S. Pat. Nos. 5,837,832, 5,874,219, and 5,856,174. Further, such a gene chip with polynucleotides of the present invention attached may be used to identify polymorphisms between the polynucleotide sequences, with polynucleotides isolated from a test subject. The knowledge of such polymorphisms (i.e. their location, as well as, their existence) would be beneficial in identifying disease loci for many disorders, including proliferative diseases and conditions. Such a method is described in U.S. Pat. Nos. 5,858,659 and 5,856,104. The US Patents referenced supra are hereby incorporated by reference in their entirety herein.

The present invention encompasses polynucleotides of the present invention that are chemically synthesized, or reproduced as peptide nucleic acids (PNA), or according to other methods known in the art. The use of PNAs would serve as the preferred form if the polynucleotides are incorporated onto a solid support, or gene chip. For the purposes of the present invention, a peptide nucleic acid (PNA) is a polyamide type of DNA analog and the monomeric units for adenine, guanine, thymine and cytosine are available commercially (Perceptive Biosystems). Certain components of DNA, such as phosphorus, phosphorus oxides, or deoxyribose derivatives, are not present in PNAs. As disclosed by P. E. Nielsen, M. Egholm, R. H. Berg and O. Buchardt, Science 254, 1497 (1991); and M. Egholm, O. Buchardt, L. Christensen, C. Behrens, S. M. Freier, D. A. Driver, R. H. Berg, S. K. Kim, B. Norden, and P. E. Nielsen, Nature 365, 666 (1993), PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the stronger binding characteristics of PNA:DNA hybrids. In addition, it is more likely that single base mismatches can be determined with PNA/DNA hybridization because a single mismatch in a PNA/DNA 15-mer lowers the melting point (T.sub.m) by 8°-20° C., vs. 4°-16° C. for the DNA/DNA 15-mer duplex. Also, the absence of charge groups in PNA means that hybridization can be done at low ionic strengths and reduce possible interference by salt during the analysis.

In addition to the foregoing, a polynucleotide can be used to control gene expression through triple helix formation or antisense DNA or RNA. Antisense techniques are discussed, for example, in Okano, J. Neurochem. 56: 560 (1991); “Oligodeoxynucleotides as Antisense Inhibitors of Gene Expression, CRC Press, Boca Raton, Fla. (1988). Triple helix formation is discussed in, for instance Lee et al., Nucleic Acids Research 6:3073 (1979); Cooney et al., Science 241:456 (1988); and Dervan et al., Science 251:1360 (1991). Both methods rely on binding of the polynucleotide to a complementary DNA or RNA. For these techniques, preferred polynucleotides are usually oligonucleotides 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991) ) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat or prevent disease.

The present invention encompasses the addition of a nuclear localization signal, operably linked to the 5′ end, 3′ end, or any location therein, to any of the oligonucleotides, antisense oligonucleotides, triple helix oligonucleotides, ribozymes, PNA oligonucleotides, and/or polynucleotides, of the present invention. See, for example, G. Cutrona, et al., Nat. Biotech., 18:300-303, (2000); which is hereby incorporated herein by reference.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell. In one example, polynucleotide sequences of the present invention may be used to construct chimeric RNA/DNA oligonucleotides corresponding to said sequences, specifically designed to induce host cell mismatch repair mechanisms in an organism upon systemic injection, for example (Bartlett, R. J., et al., Nat. Biotech, 18:615-622 (2000), which is hereby incorporated by reference herein in its entirety). Such RNA/DNA oligonucleotides could be designed to correct genetic defects in certain host strains, and/or to introduce desired phenotypes in the host (e.g., introduction of a specific polymorphism within an endogenous gene corresponding to a polynucleotide of the present invention that may ameliorate and/or prevent a disease symptom and/or disorder, etc.). Alternatively, the polynucleotide sequence of the present invention may be used to construct duplex oligonucleotides corresponding to said sequence, specifically designed to correct genetic defects in certain host strains, and/or to introduce desired phenotypes into the host (e.g., introduction of a specific polymorphism within an endogenous gene corresponding to a polynucleotide of the present invention that may ameliorate and/or prevent a disease symptom and/or disorder, etc). Such methods of using duplex oligonucleotides are known in the art and are encompassed by the present invention (see EP1007712, which is hereby incorporated by reference herein in its entirety).

The polynucleotides are also useful for identifying organisms from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The polynucleotides of the present invention can be used as additional DNA markers for RFLP.

The polynucleotides of the present invention can also be used as an alternative to RFLP, by determining the actual base-by-base DNA sequence of selected portions of an organisms genome. These sequences can be used to prepare PCR primers for amplifying and isolating such selected DNA, which can then be sequenced. Using this technique, organisms can be identified because each organism will have a unique set of DNA sequences. Once an unique ID database is established for an organism, positive identification of that organism, living or dead, can be made from extremely small tissue samples. Similarly, polynucleotides of the present invention can be used as polymorphic markers, in addition to, the identification of transformed or non-transformed cells and/or tissues.

There is also a need for reagents capable of identifying the source of a particular tissue. Such need arises, for example, when presented with tissue of unknown origin. Appropriate reagents can comprise, for example, DNA probes or primers specific to particular tissue prepared from the sequences of the present invention. Panels of such reagents can identify tissue by species and/or by organ type. In a similar fashion, these reagents can be used to screen tissue cultures for contamination. Moreover, as mentioned above, such reagents can be used to screen and/or identify transformed and non-transformed cells and/or tissues.

In the very least, the polynucleotides of the present invention can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

### Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell . Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, 121I), carbon (14C), sulfur (35S), tritium (3H), indium (121In), and technetium (99 mTc), and fluorescent labels, such as fluorescein and rhodamine, and biotin.

In addition to assaying protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, 131I, 112In, 99 mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

Thus, the invention provides a diagnostic method of a disorder, which involves (a) assaying the expression of a polypeptide of the present invention in cells or body fluid of an individual; (b) comparing the level of gene expression with a standard gene expression level, whereby an increase or decrease in the assayed polypeptide gene expression level compared to the standard expression level is indicative of a disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Moreover, polypeptides of the present invention can be used to treat, prevent, and/or diagnose disease. For example, patients can be administered a polypeptide of the present invention in an effort to replace absent or decreased levels of the polypeptide (e.g., insulin), to supplement absent or decreased levels of a different polypeptide (e.g., hemoglobin S for hemoglobin B, SOD, catalase, DNA repair proteins), to inhibit the activity of a polypeptide (e.g., an oncogene or tumor suppressor), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth inhibition, enhancement of the immune response to proliferative cells or tissues).

Similarly, antibodies directed to a polypeptide of the present invention can also be used to treat, prevent, and/or diagnose disease. For example, administration of an antibody directed to a polypeptide of the present invention can bind and reduce overproduction of the polypeptide. Similarly, administration of an antibody can activate the polypeptide, such as by binding to a polypeptide bound to a membrane (receptor).

At the very least, the polypeptides of the present invention can be used as molecular weight markers on SDS-PAGE gels or on molecular sieve gel filtration columns using methods well known to those of skill in the art. Polypeptides can also be used to raise antibodies, which in turn are used to measure protein expression from a recombinant cell, as a way of assessing transformation of the host cell. Moreover, the polypeptides of the present invention can be used to test the following biological activities.

### Gene Therapy Methods

Another aspect of the present invention is to gene therapy methods for treating or preventing disorders, diseases and conditions. The gene therapy methods relate to the introduction of nucleic acid (DNA, RNA and antisense DNA or RNA) sequences into an animal to achieve expression of a polypeptide of the present invention. This method requires a polynucleotide which codes for a polypeptide of the invention that operatively linked to a promoter and any other genetic elements necessary for the expression of the polypeptide by the target tissue. Such gene therapy and delivery techniques are known in the art, see, for example, WO90/11092, which is herein incorporated by reference.

Thus, for example, cells from a patient may be engineered with a polynucleotide (DNA or RNA) comprising a promoter operably linked to a polynucleotide of the invention ex vivo, with the engineered cells then being provided to a patient to be treated with the polypeptide. Such methods are well-known in the art. For example, see Belldegrun et al., J. Natl. Cancer Inst., 85:207-216 (1993); Ferrantini et al., Cancer Research, 53:107-1112 (1993); Ferrantini et al., J. Immunology 153: 4604-4615 (1994); Kaido, T., et al., Int. J. Cancer 60: 221-229 (1995); Ogura et al., Cancer Research 50: 5102-5106 (1990); Santodonato, et al., Human Gene Therapy 7:1-10 (1996); Santodonato, et al., Gene Therapy 4:1246-1255 (1997); and Zhang, et al., Cancer Gene Therapy 3: 31-38 (1996)), which are herein incorporated by reference. In one embodiment, the cells which are engineered are arterial cells. The arterial cells may be reintroduced into the patient through direct injection to the artery, the tissues surrounding the artery, or through catheter injection.

As discussed in more detail below, the polynucleotide constructs can be delivered by any method that delivers injectable materials to the cells of an animal, such as, injection into the interstitial space of tissues (heart, muscle, skin, lung, liver, and the like). The polynucleotide constructs may be delivered in a pharmaceutically acceptable liquid or aqueous carrier.

In one embodiment, the polynucleotide of the invention is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, the polynucleotides of the invention can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The polynucleotide vector constructs of the invention used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1/V5, pcDNA3.1, and pRc/CMV2 available from Invitrogen. Other suitable vectors will be readily apparent to the skilled artisan.

Any strong promoter known to those skilled in the art can be used for driving the expression of polynucleotide sequence of the invention. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter also may be the native promoter for the polynucleotides of the invention.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct of the invention can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

The constructs may also be delivered with delivery vehicles such as viral sequences, viral particles, liposome formulations, lipofectin, precipitating agents, etc. Such methods of delivery are known in the art.

In certain embodiments, the polynucleotide constructs of the invention are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA, 84:7413-7416 (1987), which is herein incorporated by reference); mRNA (Malone et al., Proc. Natl. Acad. Sci. USA , 86:6077-6081 (1989), which is herein incorporated by reference); and purified transcription factors (Debs et al., J. Biol. Chem . . . , 265:10189-10192 (1990), which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Feigner et al., Proc. Natl. Acad. Sci. USA , 84:7413-7416 (1987), which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication NO: WO 90/11092 (which is herein incorporated by reference) for a description of the synthesis of DOTAP (1,2-bis (oleoyloxy)-3-(trimethylammonio)propane) liposomes. Preparation of DOTMA liposomes is explained in the literature, see, e.g., Felgner et al., Proc. Natl. Acad. Sci. USA, 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology, 101:512-527 (1983), which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca2+-EDTA chelation (Papahadjopoulos et al., Biochim. Biophys. Acta, 394:483 (1975); Wilson et al., Cell , 17:77 (1979)); ether injection (Deamer et al., Biochim. Biophys. Acta, 443:629 (1976); Ostro et al., Biochem. Biophys. Res. Commun., 76:836 (1977); Fraley et al., Proc. Natl. Acad. Sci. USA, 76:3348 (1979)); detergent dialysis (Enoch et al., Proc. Natl. Acad. Sci. USA , 76:145 (1979)); and reverse-phase evaporation (REV) (Fraley et al., J. Biol. Chem. . . . 255:10431 (1980); Szoka et al., Proc. Natl. Acad. Sci. USA , 75:145 (1978); Schaefer-Ridder et al., Science, 215:166 (1982)), which are herein incorporated by reference.

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals.

In certain embodiments, cells are engineered, ex vivo or in vivo, using a retroviral particle containing RNA which comprises a sequence encoding polypeptides of the invention. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14X, VT-19-17-H2, RCRE, RCRIP, GP+E-86, GP+envAm12, and DAN cell lines as described in Miller, Human Gene Therapy , 1:5-14 (1990), which is incorporated herein by reference in its entirety. The vector may transduce the packaging cells through any means known in the art. Such means include, but are not limited to, electroporation, the use of liposomes, and CaPO4 precipitation. In one alternative, the retroviral plasmid vector may be encapsulated into a liposome, or coupled to a lipid, and then administered to a host.

The producer cell line generates infectious retroviral vector particles which include polynucleotide encoding polypeptides of the invention. Such retroviral vector particles then may be employed, to transduce eukaryotic cells, either in vitro or in vivo. The transduced eukaryotic cells will express polypeptides of the invention.

In certain other embodiments, cells are engineered, ex vivo or in vivo, with polynucleotides of the invention contained in an adenovirus vector. Adenovirus can be manipulated such that it encodes and expresses polypeptides of the invention, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartzet al., Am. Rev. Respir. Dis., 109:233-238 (1974)). Finally, adenovirus mediated gene transfer has been demonstrated in a number of instances including transfer of alpha-1-antitrypsin and CFTR to the lungs of cotton rats (Rosenfeld et al., Science, 252:431-434 (1991); Rosenfeld et al., Cell, 68:143-155 (1992)). Furthermore, extensive studies to attempt to establish adenovirus as a causative agent in human cancer were uniformly negative (Green et al. Proc. Natl. Acad. Sci. USA, 76:6606 (1979)).

Suitable adenoviral vectors useful in the present invention are described, for example, in Kozarsky and Wilson, Curr. Opin. Genet. Devel., 3:499-503 (1993); Rosenfeld et al., Cell , 68:143-155 (1992); Engelhardt et al., Human Genet. Ther., 4:759-769 (1993); Yang et al., Nature Genet., 7:362-369 (1994); Wilson et al., Nature, 365:691-692 (1993); and U.S. Pat. No. 5,652,224, which are herein incorporated by reference. For example, the adenovirus vector Ad2 is useful and can be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, Curr. Topics in Microbiol. Immunol., 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The polynucleotide construct containing polynucleotides of the invention is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct of the invention. These viral particles are then used to transduce eukaryotic cells, either ex vivo or in vivo. The transduced cells will contain the polynucleotide construct integrated into its genome, and will express the desired gene product.

Another method of gene therapy involves operably associating heterologous control regions and endogenous polynucleotide sequences (e.g. encoding the polypeptide sequence of interest) via homologous recombination (see, e.g., U.S. Pat. No. 5,641,670, issued Jun. 24, 1997; International Publication NO: WO 96/29411, published Sep. 26, 1996; International Publication NO: WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous sequence is placed under the control of the promoter. The promoter then drives the expression of the endogenous sequence.

The polynucleotides encoding polypeptides of the present invention may be administered along with other polynucleotides encoding angiogenic proteins. Angiogenic proteins include, but are not limited to, acidic and basic fibroblast growth factors, VEGF-1, VEGF-2 (VEGF-C), VEGF-3 (VEGF-B), epidermal growth factor alpha and beta, platelet-derived endothelial cell growth factor, platelet-derived growth factor, tumor necrosis factor alpha, hepatocyte growth factor, insulin like growth factor, colony stimulating factor, macrophage colony stimulating factor, granulocyte/macrophage colony stimulating factor, and nitric oxide synthase.

Preferably, the polynucleotide encoding a polypeptide of the invention contains a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers. (Kaneda et al., Science, 243:375 (1989)).

A preferred method of local administration is by direct injection. Preferably, a recombinant molecule of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA, 189:11277-11281 (1992), which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian. Therapeutic compositions of the present invention can be administered to any animal, preferably to mammals and birds. Preferred mammals include humans, dogs, cats, mice, rats, rabbits sheep, cattle, horses and pigs, with humans being particularly preferred.

### Cardiovascular Disorders

Polynucleotides or polypeptides, or agonists or antagonists of the invention may be used to treat, prevent, and/or diagnose cardiovascular diseases, disorders, and/or conditions, including peripheral artery disease, such as limb ischemia.

Cardiovascular diseases, disorders, and/or conditions include cardiovascular abnormalities, such as arterio-arterial fistula, arteriovenous fistula, cerebral arteriovenous malformations, congenital heart defects, pulmonary atresia, and Scimitar Syndrome. Congenital heart defects include aortic coarctation, cor triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as aortopulmonary septal defect, endocardial cushion defects, Lutembacher's Syndrome, trilogy of Fallot, ventricular heart septal defects.

Cardiovascular diseases, disorders, and/or conditions also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, Scimitar Syndrome, cardiovascular syphilis, and cardiovascular tuberculosis.

Arrhythmias include sinus arrhythmia, atrial fibrillation, atrial flutter, bradycardia, extrasystole, Adams-Stokes Syndrome, bundle-branch block, sinoatrial block, long QT syndrome, parasystole, Lown-Ganong-Levine Syndrome, Mahaim-type pre-excitation syndrome, Wolff-Parkinson-White syndrome, sick sinus syndrome, tachycardias, and ventricular fibrillation. Tachycardias include paroxysmal tachycardia, supraventricular tachycardia, accelerated idioventricular rhythm, atrioventricular nodal reentry tachycardia, ectopic atrial tachycardia, ectopic junctional tachycardia, sinoatrial nodal reentry tachycardia, sinus tachycardia, Torsades de Pointes, and ventricular tachycardia.

Heart valve disease include aortic valve insufficiency, aortic valve stenosis, hear murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis.

Myocardial diseases include alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis.

Myocardial ischemias include coronary disease, such as angina pectoris, coronary aneurysm, coronary arteriosclerosis, coronary thrombosis, coronary vasospasm, myocardial infarction and myocardial stunning.

Cardiovascular diseases also include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular diseases, disorders, and/or conditions, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

Aneurysms include dissecting aneurysms, false aneurysms, infected aneurysms, ruptured aneurysms, aortic aneurysms, cerebral aneurysms, coronary aneurysms, heart aneurysms, and iliac aneurysms.

Arterial occlusive diseases include arteriosclerosis, intermittent claudication, carotid stenosis, fibromuscular dysplasias, mesenteric vascular occlusion, Moyamoya disease, renal artery obstruction, retinal artery occlusion, and thromboangiitis obliterans.

Cerebrovascular diseases, disorders, and/or conditions include carotid artery diseases, cerebral amyloid angiopathy, cerebral aneurysm, cerebral anoxia, cerebral arteriosclerosis, cerebral arteriovenous malformation, cerebral artery diseases, cerebral embolism and thrombosis, carotid artery thrombosis, sinus thrombosis, Wallenberg's syndrome, cerebral hemorrhage, epidural hematoma, subdural hematoma, subaraxhnoid hemorrhage, cerebral infarction, cerebral ischemia (including transient), subclavian steal syndrome, periventricular leukomalacia, vascular headache, cluster headache, migraine, and vertebrobasilar insufficiency.

Embolisms include air embolisms, amniotic fluid embolisms, cholesterol embolisms, blue toe syndrome, fat embolisms, pulmonary embolisms, and thromoboembolisms. Thrombosis include coronary thrombosis, hepatic vein thrombosis, retinal vein occlusion, carotid artery thrombosis, sinus thrombosis, Wallenberg's syndrome, and thrombophlebitis.

Ischemia includes cerebral ischemia, ischemic colitis, compartment syndromes, anterior compartment syndrome, myocardial ischemia, reperfusion injuries, and peripheral limb ischemia. Vasculitis includes aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis.

Polynucleotides or polypeptides, or agonists or antagonists of the invention, are especially effective for the treatment of critical limb ischemia and coronary disease.

Polypeptides may be administered using any method known in the art, including, but not limited to, direct needle injection at the delivery site, intravenous injection, topical administration, catheter infusion, biolistic injectors, particle accelerators, gelfoam sponge depots, other commercially available depot materials, osmotic pumps, oral or suppositorial solid pharmaceutical formulations, decanting or topical applications during surgery, aerosol delivery. Such methods are known in the art. Polypeptides of the invention may be administered as part of a Therapeutic, described in more detail below. Methods of delivering polynucleotides of the invention are described in more detail herein.

### Anti-Angiogenesis Activity

The naturally occurring balance between endogenous stimulators and inhibitors of angiogenesis is one in which inhibitory influences predominate. Rastinejad et al., Cell 56:345-355 (1989). In those rare instances in which neovascularization occurs under normal physiological conditions, such as wound healing, organ regeneration, embryonic development, and female reproductive processes, angiogenesis is stringently regulated and spatially and temporally delimited. Under conditions of pathological angiogenesis such as that characterizing solid tumor growth, these regulatory controls fail. Unregulated angiogenesis becomes pathologic and sustains progression of many neoplastic and non-neoplastic diseases. A number of serious diseases are dominated by abnormal neovascularization including solid tumor growth and metastases, arthritis, some types of eye diseases, disorders, and/or conditions, and psoriasis. See, e.g., reviews by Moses et al., Biotech. 9:630-634 (1991); Folkman et al., N. Engl. J. Med., 333:1757-1763 (1995); Auerbach et al., J. Microvasc. Res. 29:401-411 (1985); Folkman, Advances in Cancer Research, eds. Klein and Weinhouse, Academic Press, New York, pp. 175-203 (1985); Patz, Am. J. Opthalmol. 94:715-743 (1982); and Folkman et al., Science 221:719-725 (1983). In a number of pathological conditions, the process of angiogenesis contributes to the disease state. For example, significant data have accumulated which suggest that the growth of solid tumors is dependent on angiogenesis. Folkman and Klagsbrun, Science 235:442-447 (1987).

The present invention provides for treatment of diseases, disorders, and/or conditions associated with neovascularization by administration of the polynucleotides and/or polypeptides of the invention, as well as agonists or antagonists of the present invention. Malignant and metastatic conditions which can be treated with the polynucleotides and polypeptides, or agonists or antagonists of the invention include, but are not limited to, malignancies, solid tumors, and cancers described herein and otherwise known in the art (for a review of such disorders, see Fishman et al., Medicine, 2d Ed., J. B. Lippincott Co., Philadelphia (1985)). Thus, the present invention provides a method of treating, preventing, and/or diagnosing an angiogenesis-related disease and/or disorder, comprising administering to an individual in need thereof a therapeutically effective amount of a polynucleotide, polypeptide, antagonist and/or agonist of the invention. For example, polynucleotides, polypeptides, antagonists and/or agonists may be utilized in a variety of additional methods in order to therapeutically treat or prevent a cancer or tumor. Cancers which may be treated, prevented, and/or diagnosed with polynucleotides, polypeptides, antagonists and/or agonists include, but are not limited to solid tumors, including prostate, lung, breast, ovarian, stomach, pancreas, larynx, esophagus, testes, liver, parotid, biliary tract, colon, rectum, cervix, uterus, endometrium, kidney, bladder, thyroid cancer; primary tumors and metastases; melanomas; glioblastoma; Kaposi's sarcoma; leiomyosarcoma; non-small cell lung cancer; colorectal cancer; advanced malignancies; and blood born tumors such as leukemias. For example, polynucleotides, polypeptides, antagonists and/or agonists may be delivered topically, in order to treat or prevent cancers such as skin cancer, head and neck tumors, breast tumors, and Kaposi's sarcoma.

Within yet other aspects, polynucleotides, polypeptides, antagonists and/or agonists may be utilized to treat superficial forms of bladder cancer by, for example, intravesical administration. Polynucleotides, polypeptides, antagonists and/or agonists may be delivered directly into the tumor, or near the tumor site, via injection or a catheter. Of course, as the artisan of ordinary skill will appreciate, the appropriate mode of administration will vary according to the cancer to be treated. Other modes of delivery are discussed herein.

Polynucleotides, polypeptides, antagonists and/or agonists may be useful in treating, preventing, and/or diagnosing other diseases, disorders, and/or conditions, besides cancers, which involve angiogenesis. These diseases, disorders, and/or conditions include, but are not limited to: benign tumors, for example hemangiomas, acoustic neuromas, neurofibromas, trachomas, and pyogenic granulomas; artheroscleric plaques; ocular angiogenic diseases, for example, diabetic retinopathy, retinopathy of prematurity, macular degeneration, corneal graft rejection, neovascular glaucoma, retrolental fibroplasia, rubeosis, retinoblastoma, uvietis and Pterygia (abnormal blood vessel growth) of the eye; rheumatoid arthritis; psoriasis; delayed wound healing; endometriosis; vasculogenesis; granulations; hypertrophic scars (keloids); nonunion fractures; scleroderma; trachoma; vascular adhesions; myocardial angiogenesis; coronary collaterals; cerebral collaterals; arteriovenous malformations; ischemic limb angiogenesis; Osler-Webber Syndrome; plaque neovascularization; telangiectasia; hemophiliac joints; angiofibroma; fibromuscular dysplasia; wound granulation; Crohn's disease; and atherosclerosis.

For example, within one aspect of the present invention methods are provided for treating, preventing, and/or diagnosing hypertrophic scars and keloids, comprising the step of administering a polynucleotide, polypeptide, antagonist and/or agonist of the invention to a hypertrophic scar or keloid.

Within one embodiment of the present invention polynucleotides, polypeptides, antagonists and/or agonists are directly injected into a hypertrophic scar or keloid, in order to prevent the progression of these lesions. This therapy is of particular value in the prophylactic treatment of conditions which are known to result in the development of hypertrophic scars and keloids (e.g., burns), and is preferably initiated after the proliferative phase has had time to progress (approximately 14 days after the initial injury), but before hypertrophic scar or keloid development. As noted above, the present invention also provides methods for treating, preventing, and/or diagnosing neovascular diseases of the eye, including for example, corneal neovascularization, neovascular glaucoma, proliferative diabetic retinopathy, retrolental fibroplasia and macular degeneration.

Moreover, Ocular diseases, disorders, and/or conditions associated with neovascularization which can be treated, prevented, and/or diagnosed with the polynucleotides and polypeptides of the present invention (including agonists and/or antagonists) include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. See, e.g., reviews by Waltman et al., Am. J. Ophthal. 85:704-710 (1978) and Gartner et al., Surv. Ophthal. 22:291-312 (1978).

Thus, within one aspect of the present invention methods are provided for treating or preventing neovascular diseases of the eye such as corneal neovascularization (including corneal graft neovascularization), comprising the step of administering to a patient a therapeutically effective amount of a compound (as described above) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates. A wide variety of diseases, disorders, and/or conditions can result in corneal neovascularization, including for example, corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of wearing contact lenses.

Within particularly preferred embodiments of the invention, may be prepared for topical administration in saline (combined with any of the preservatives and antimicrobial agents commonly used in ocular preparations), and administered in eyedrop form. The solution or suspension may be prepared in its pure form and administered several times daily. Alternatively, anti-angiogenic compositions, prepared as described above, may also be administered directly to the cornea. Within preferred embodiments, the anti-angiogenic composition is prepared with a muco-adhesive polymer which binds to cornea. Within further embodiments, the anti-angiogenic factors or anti-angiogenic compositions may be utilized as an adjunct to conventional steroid therapy. Topical therapy may also be useful prophylactically in corneal lesions which are known to have a high probability of inducing an angiogenic response (such as chemical burns). In these instances the treatment, likely in combination with steroids, may be instituted immediately to help prevent subsequent complications.

Within other embodiments, the compounds described above may be injected directly into the corneal stroma by an ophthalmologist under microscopic guidance. The preferred site of injection may vary with the morphology of the individual lesion, but the goal of the administration would be to place the composition at the advancing front of the vasculature (i.e., interspersed between the blood vessels and the normal cornea). In most cases this would involve perilimbic corneal injection to “protect” the cornea from the advancing blood vessels. This method may also be utilized shortly after a corneal insult in order to prophylactically prevent corneal neovascularization. In this situation the material could be injected in the perilimbic cornea interspersed between the corneal lesion and its undesired potential limbic blood supply. Such methods may also be utilized in a similar fashion to prevent capillary invasion of transplanted corneas. In a sustained-release form injections might only be required 2-3 times per year. A steroid could also be added to the injection solution to reduce inflammation resulting from the injection itself.

Within another aspect of the present invention, methods are provided for treating or preventing neovascular glaucoma, comprising the step of administering to a patient a therapeutically effective amount of a polynucleotide, polypeptide, antagonist and/or agonist to the eye, such that the formation of blood vessels is inhibited. In one embodiment, the compound may be administered topically to the eye in order to treat or prevent early forms of neovascular glaucoma. Within other embodiments, the compound may be implanted by injection into the region of the anterior chamber angle. Within other embodiments, the compound may also be placed in any location such that the compound is continuously released into the aqueous humor. Within another aspect of the present invention, methods are provided for treating or preventing proliferative diabetic retinopathy, comprising the step of administering to a patient a therapeutically effective amount of a polynucleotide, polypeptide, antagonist and/or agonist to the eyes, such that the formation of blood vessels is inhibited.

Within particularly preferred embodiments of the invention, proliferative diabetic retinopathy may be treated by injection into the aqueous humor or the vitreous, in order to increase the local concentration of the polynucleotide, polypeptide, antagonist and/or agonist in the retina. Preferably, this treatment should be initiated prior to the acquisition of severe disease requiring photocoagulation.

Within another aspect of the present invention, methods are provided for treating or preventing retrolental fibroplasia, comprising the step of administering to a patient a therapeutically effective amount of a polynucleotide, polypeptide, antagonist and/or agonist to the eye, such that the formation of blood vessels is inhibited. The compound may be administered topically, via intravitreous injection and/or via intraocular implants.

Additionally, diseases, disorders, and/or conditions which can be treated, prevented, and/or diagnosed with the polynucleotides, polypeptides, agonists and/or agonists include, but are not limited to, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Moreover, diseases, disorders, and/or conditions and/or states, which can be treated, prevented, and/or diagnosed with the polynucleotides, polypeptides, agonists and/or agonists include, but are not limited to, solid tumors, blood born tumors such as leukemias, tumor metastasis, Kaposi's sarcoma, benign tumors, for example hemangiomas, acoustic neuromas, neurofibromas, trachomas, and pyogenic granulomas, rheumatoid arthritis, psoriasis, ocular angiogenic diseases, for example, diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vascluogenesis, granulations, hypertrophic scars (keloids), nonunion fractures, scleroderma, trachoma, vascular adhesions, myocardial angiogenesis, coronary collaterals, cerebral collaterals, arteriovenous malformations, ischemic limb angiogenesis, Osler-Webber Syndrome, plaque neovascularization, telangiectasia, hemophiliac joints, angiofibroma fibromuscular dysplasia, wound granulation, Crohn's disease, atherosclerosis, birth control agent by preventing vascularization required for embryo implantation controlling menstruation, diseases that have angiogenesis as a pathologic consequence such as cat scratch disease (Rochele minalia quintosa), ulcers (Helicobacter pylori), Bartonellosis and bacillary angiomatosis.

In one aspect of the birth control method, an amount of the compound sufficient to block embryo implantation is administered before or after intercourse and fertilization have occurred, thus providing an effective method of birth control, possibly a “morning after” method. Polynucleotides, polypeptides, agonists and/or agonists may also be used in controlling menstruation or administered as either a peritoneal lavage fluid or for peritoneal implantation in the treatment of endometriosis.

Polynucleotides, polypeptides, agonists and/or agonists of the present invention may be incorporated into surgical sutures in order to prevent stitch granulomas.

Polynucleotides, polypeptides, agonists and/or agonists may be utilized in a wide variety of surgical procedures. For example, within one aspect of the present invention a compositions (in the form of, for example, a spray or film) may be utilized to coat or spray an area prior to removal of a tumor, in order to isolate normal surrounding tissues from malignant tissue, and/or to prevent the spread of disease to surrounding tissues. Within other aspects of the present invention, compositions (e.g., in the form of a spray) may be delivered via endoscopic procedures in order to coat tumors, or inhibit angiogenesis in a desired locale. Within yet other aspects of the present invention, surgical meshes which have been coated with anti- angiogenic compositions of the present invention may be utilized in any procedure wherein a surgical mesh might be utilized. For example, within one embodiment of the invention a surgical mesh laden with an anti-angiogenic composition may be utilized during abdominal cancer resection surgery (e.g., subsequent to colon resection) in order to provide support to the structure, and to release an amount of the anti-angiogenic factor.

Within further aspects of the present invention, methods are provided for treating tumor excision sites, comprising administering a polynucleotide, polypeptide, agonist and/or agonist to the resection margins of a tumor subsequent to excision, such that the local recurrence of cancer and the formation of new blood vessels at the site is inhibited. Within one embodiment of the invention, the anti-angiogenic compound is administered directly to the tumor excision site (e.g., applied by swabbing, brushing or otherwise coating the resection margins of the tumor with the anti-angiogenic compound). Alternatively, the anti-angiogenic compounds may be incorporated into known surgical pastes prior to administration. Within particularly preferred embodiments of the invention, the anti-angiogenic compounds are applied after hepatic resections for malignancy, and after neurosurgical operations.

Within one aspect of the present invention, polynucleotides, polypeptides, agonists and/or agonists may be administered to the resection margin of a wide variety of tumors, including for example, breast, colon, brain and hepatic tumors. For example, within one embodiment of the invention, anti-angiogenic compounds may be administered to the site of a neurological tumor subsequent to excision, such that the formation of new blood vessels at the site are inhibited.

The polynucleotides, polypeptides, agonists and/or agonists of the present invention may also be administered along with other anti-angiogenic factors. Representative examples of other anti-angiogenic factors include: Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include platelet factor 4; protamine sulphate; sulphated chitin derivatives (prepared from queen crab shells), (Murata et al., Cancer Res. 51:22-26, 1991); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, alpha,alpha-dipyridyl, aminopropionitrile fumarate; 4-propyl-5-(4-pyridinyl)-2 (3H)-oxazolone; Methotrexate; Mitoxantrone; Heparin; Interferons; 2 Macroglobulin-serum; ChIMP-3 (Pavloff et al., J. Bio. Chem. 267:17321-17326, 1992); Chymostatin (Tomkinson et al., Biochem J. 286:475-480, 1992); Cyclodextrin Tetradecasulfate; Eponemycin; Camptothecin; Fumagillin (Ingber et al., Nature 348:555-557, 1990); Gold Sodium Thiomalate (“GST”; Matsubara and Ziff, J. Clin. Invest. 79:1440-1446, 1987); anticollagenase-serum; alpha2-antiplasmin (Holmes et al., J. Biol. Chem . . . 262 (4):1659-1664, 1987); Bisantrene (National Cancer Institute); Lobenzarit disodium (N-(2)-carboxyphenyl-4-chloroanthronilic acid disodium or “CCA”; Takeuchi et al., Agents Actions 36:312-316, 1992); Thalidomide; Angostatic steroid; AGM-1470; carboxynaminolmidazole; and metalloproteinase inhibitors such as BB94.

### Chemotaxis

A polynucleotide or polypeptide and/or agonist or antagonist of the present invention may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

A polynucleotide or polypeptide and/or agonist or antagonist of the present invention may increase chemotaxic activity of particular cells. These chemotactic molecules can then be used to treat, prevent, and/or diagnose inflammation, infection, hyperproliferative diseases, disorders, and/or conditions, or any immune system disorder by increasing the number of cells targeted to a particular location in the body. For example, chemotaxic molecules can be used to treat, prevent, and/or diagnose wounds and other trauma to tissues by attracting immune cells to the injured location. Chemotactic molecules of the present invention can also attract fibroblasts, which can be used to treat, prevent, and/or diagnose wounds.

It is also contemplated that a polynucleotide or polypeptide and/or agonist or antagonist of the present invention may inhibit chemotactic activity. These molecules could also be used to treat, prevent, and/or diagnose diseases, disorders, and/or conditions. Thus, a polynucleotide or polypeptide and/or agonist or antagonist of the present invention could be used as an inhibitor of chemotaxis.

### Binding Activity

A polypeptide of the present invention may be used to screen for molecules that bind to the polypeptide or for molecules to which the polypeptide binds. The binding of the polypeptide and the molecule may activate (agonist), increase, inhibit (antagonist), or decrease activity of the polypeptide or the molecule bound. Examples of such molecules include antibodies, oligonucleotides, proteins (e.g., receptors),or small molecules.

Preferably, the molecule is closely related to the natural ligand of the polypeptide, e.g., a fragment of the ligand, or a natural substrate, a ligand, a structural or functional mimetic. (See, Coligan et al., Current Protocols in Immunology 1 (2):Chapter 5 (1991).) Similarly, the molecule can be closely related to the natural receptor to which the polypeptide binds, or at least, a fragment of the receptor capable of being bound by the polypeptide (e.g., active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

Additionally, the receptor to which a polypeptide of the invention binds can be identified by numerous methods known to those of skill in the art, for example, ligand panning and FACS sorting (Coligan, et al., Current Protocols in Immun., 1 (2), Chapter 5, (1991)). For example, expression cloning is employed wherein polyadenylated RNA is prepared from a cell responsive to the polypeptides, for example, NIH3T3 cells which are known to contain multiple receptors for the FGF family proteins, and SC-3 cells, and a cDNA library created from this RNA is divided into pools and used to transfect COS cells or other cells that are not responsive to the polypeptides. Transfected cells which are grown on glass slides are exposed to the polypeptide of the present invention, after they have been labeled. The polypeptides can be labeled by a variety of means including iodination or inclusion of a recognition site for a site-specific protein kinase.

Following fixation and incubation, the slides are subjected to auto-radiographic analysis. Positive pools are identified and sub-pools are prepared and re-transfected using an iterative sub-pooling and re-screening process, eventually yielding a single clones that encodes the putative receptor.

As an alternative approach for receptor identification, the labeled polypeptides can be photoaffinity linked with cell membrane or extract preparations that express the receptor molecule. Cross-linked material is resolved by PAGE analysis and exposed to X-ray film. The labeled complex containing the receptors of the polypeptides can be excised, resolved into peptide fragments, and subjected to protein microsequencing. The amino acid sequence obtained from microsequencing would be used to design a set of degenerate oligonucleotide probes to screen a cDNA library to identify the genes encoding the putative receptors.

Moreover, the techniques of gene-shuffling, motif-shuffling, exon-shuffling, and/or codon-shuffling (collectively referred to as “DNA shuffling”) may be employed to modulate the activities of polypeptides of the invention thereby effectively generating agonists and antagonists of polypeptides of the invention. See generally, U.S. Pat. Nos. 5,605,793, 5,811,238, 5,830,721, 5,834,252, and 5,837,458, and Patten, P. A., et al., Curr. Opinion Biotechnol. 8:724-33 (1997); Harayama, S. Trends Biotechnol. 16 (2):76-82 (1998); Hansson, L. O., et al., J. Mol. Biol. 287:265-76 (1999); and Lorenzo, M. M. and Blasco, R. Biotechniques 24 (2):308-13 (1998) (each of these patents and publications are hereby incorporated by reference). In one embodiment, alteration of polynucleotides and corresponding polypeptides of the invention may be achieved by DNA shuffling. DNA shuffling involves the assembly of two or more DNA segments into a desired polynucleotide sequence of the invention molecule by homologous, or site-specific, recombination. In another embodiment, polynucleotides and corresponding polypeptides of the invention may be altered by being subjected to random mutagenesis by error-prone PCR, random nucleotide insertion or other methods prior to recombination. In another embodiment, one or more components, motifs, sections, parts, domains, fragments, etc., of the polypeptides of the invention may be recombined with one or more components, motifs, sections, parts, domains, fragments, etc. of one or more heterologous molecules. In preferred embodiments, the heterologous molecules are family members. In further preferred embodiments, the heterologous molecule is a growth factor such as, for example, platelet-derived growth factor (PDGF), insulin-like growth factor (IGF-I), transforming growth factor (TGF)-alpha, epidermal growth factor (EGF), fibroblast growth factor (FGF), TGF-beta, bone morphogenetic protein (BMP)-2, BMP-4, BMP-5, BMP-6, BMP-7, activins A and B, decapentaplegic (dpp), 60A, OP-2, dorsalin, growth differentiation factors (GDFs), nodal, MIS, inhibin-alpha, TGF-betal, TGF-beta2, TGF-beta3, TGF-beta5, and glial-derived neurotrophic factor (GDNF).

Other preferred fragments are biologically active fragments of the polypeptides of the invention. Biologically active fragments are those exhibiting activity similar, but not necessarily identical, to an activity of the polypeptide. The biological activity of the fragments may include an improved desired activity, or a decreased undesirable activity.

Additionally, this invention provides a method of screening compounds to identify those which modulate the action of the polypeptide of the present invention. An example of such an assay comprises combining a mammalian fibroblast cell, a the polypeptide of the present invention, the compound to be screened and 3[H] thymidine under cell culture conditions where the fibroblast cell would normally proliferate. A control assay may be performed in the absence of the compound to be screened and compared to the amount of fibroblast proliferation in the presence of the compound to determine if the compound stimulates proliferation by determining the uptake of 3[H] thymidine in each case. The amount of fibroblast cell proliferation is measured by liquid scintillation chromatography which measures the incorporation of 3[H] thymidine. Both agonist and antagonist compounds may be identified by this procedure.

In another method, a mammalian cell or membrane preparation expressing a receptor for a polypeptide of the present invention is incubated with a labeled polypeptide of the present invention in the presence of the compound. The ability of the compound to enhance or block this interaction could then be measured. Alternatively, the response of a known second messenger system following interaction of a compound to be screened and the receptor is measured and the ability of the compound to bind to the receptor and elicit a second messenger response is measured to determine if the compound is a potential agonist or antagonist. Such second messenger systems include but are not limited to, cAMP guanylate cyclase, ion channels or phosphoinositide hydrolysis.

All of these above assays can be used as diagnostic or prognostic markers. The molecules discovered using these assays can be used to treat, prevent, and/or diagnose disease or to bring about a particular result in a patient (e.g., blood vessel growth) by activating or inhibiting the polypeptide/molecule. Moreover, the assays can discover agents which may inhibit or enhance the production of the polypeptides of the invention from suitably manipulated cells or tissues. Therefore, the invention includes a method of identifying compounds which bind to the polypeptides of the invention comprising the steps of: (a) incubating a candidate binding compound with the polypeptide; and (b) determining if binding has occurred. Moreover, the invention includes a method of identifying agonists/antagonists comprising the steps of: (a) incubating a candidate compound with the polypeptide, (b) assaying a biological activity, and (b) determining if a biological activity of the polypeptide has been altered.

Also, one could identify molecules bind a polypeptide of the invention experimentally by using the beta-pleated sheet regions contained in the polypeptide sequence of the protein. Accordingly, specific embodiments of the invention are directed to polynucleotides encoding polypeptides which comprise, or alternatively consist of, the amino acid sequence of each beta pleated sheet regions in a disclosed polypeptide sequence. Additional embodiments of the invention are directed to polynucleotides encoding polypeptides which comprise, or alternatively consist of, any combination or all of contained in the polypeptide sequences of the invention. Additional preferred embodiments of the invention are directed to polypeptides which comprise, or alternatively consist of, the amino acid sequence of each of the beta pleated sheet regions in one of the polypeptide sequences of the invention. Additional embodiments of the invention are directed to polypeptides which comprise, or alternatively consist of, any combination or all of the beta pleated sheet regions in one of the polypeptide sequences of the invention.

### Targeted Delivery

In another embodiment, the invention provides a method of delivering compositions to targeted cells expressing a receptor for a polypeptide of the invention, or cells expressing a cell bound form of a polypeptide of the invention.

As discussed herein, polypeptides or antibodies of the invention may be associated with heterologous polypeptides, heterologous nucleic acids, toxins, or prodrugs via hydrophobic, hydrophilic, ionic and/or covalent interactions. In one embodiment, the invention provides a method for the specific delivery of compositions of the invention to cells by administering polypeptides of the invention (including antibodies) that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides a method for the specific destruction of cells (e.g., the destruction of tumor cells) by administering polypeptides of the invention (e.g., polypeptides of the invention or antibodies of the invention) in association with toxins or cytotoxic prodrugs.

By “toxin” is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. By “cytotoxic prodrug” is meant a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

### Antisense And Ribozyme (Antagonists)

In specific embodiments, antagonists according to the present invention are nucleic acids corresponding to the sequences contained in SEQ ID NO:X, or the complementary strand thereof. In one embodiment, antisense sequence is generated internally by the organism, in another embodiment, the antisense sequence is separately administered (see, for example, O'Connor, Neurochem., 56:560 (1991). Oligodeoxynucleotides as Antisense Inhibitors of Gene Expression, CRC Press, Boca Raton, Fla. (1988). Antisense technology can be used to control gene expression through antisense DNA or RNA, or through triple-helix formation. Antisense techniques are discussed for example, in Okano, Neurochem., 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research, 6:3073 (1979); Cooney et al., Science, 241:456 (1988); and Dervan et al., Science, 251:1300 (1991). The methods are based on binding of a polynucleotide to a complementary DNA or RNA.

For example, the use of c-myc and c-myb antisense RNA constructs to inhibit the growth of the non-lymphocytic leukemia cell line HL-60 and other cell lines was previously described. (Wickstrom et al. (1988); Anfossi et al. (1989)). These experiments were performed in vitro by incubating cells with the oligoribonucleotide. A similar procedure for in vivo use is described in WO 91/15580. Briefly, a pair of oligonucleotides for a given antisense RNA is produced as follows: A sequence complimentary to the first 15 bases of the open reading frame is flanked by an EcoR1 site on the 5 end and a HindlIl site on the 3 end. Next, the pair of oligonucleotides is heated at 90° C. for one minute and then annealed in 2× ligation buffer (20 mM TRIS HCl pH 7.5, 10 mM MgCl2, 10 MM dithiothreitol (DTT) and 0.2 mM ATP) and then ligated to the EcoR1/Hind III site of the retroviral vector PMV7 (WO 91/15580).

For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the antisense nucleic acid of the invention is produced intracellularly by transcription from an exogenous sequence. For example, a vector or a portion thereof, is transcribed, producing an antisense nucleic acid (RNA) of the invention. Such a vector would contain a sequence encoding the antisense nucleic acid of the invention. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the sequence encoding a polypeptide of the invention, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature, 29:304-310 (1981), the promoter contained in the 3′ long terminal repeat of Rous sarcoma virus (Yamamoto et al., Cell, 22:787-797 (1980), the herpes thymidine promoter (Wagner et al., Proc. NatI. Acad. Sci. U.S.A., 78:1441-1445 (1981), the regulatory sequences of the metallothionein gene (Brinster et al., Nature, 296:39-42 (1982)), etc.

The antisense nucleic acids of the invention comprise a sequence complementary to at least a portion of an RNA transcript of a gene of interest. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded antisense nucleic acids of the invention, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with a RNA sequence of the invention it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., Nature, 372:333-335 (1994). Thus, oligonucleotides complementary to either the 5′- or 3′-non-translated, non-coding regions of a polynucleotide sequence of the invention could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The polynucleotides of the invention can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci., 84:648-652 (1987); PCT Publication NO: WO88/09810, published Dec. 15, 1988) or the blood-brain barrier (see, e.g., PCT Publication NO: WO89/10134, published Apr. 25, 1988), hybridization-triggered cleavage agents. (See, e.g., Krol et al., BioTechniques, 6:958-976 (1988)) or intercalating agents. (See, e.g., Zon, Pharm. Res., 5:539-549 (1988)). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an a-anomeric oligonucleotide. An a-anomeric oligonucleotide forms specific double-stranded hybrids with complementary RNA in which, contrary to the usual b-units, the strands run parallel to each other (Gautier et al., Nucl. Acids Res., 15:6625-6641 (1987)). The oligonucleotide is a 2-0-methylribonucleotide (Inoue et al., Nucl. Acids Res., 15:6131-6148 (1987)), or a chimeric RNA-DNA analogue (Inoue et al., FEBS Lett. 215:327-330 (1987)).

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (Nucl. Acids Res., 16:3209 (1988)), methylphosphonate oligonucleotides can be prepared by use of controlled pore glass polymer supports (Sarin et al., Proc. Natl. Acad. Sci. U.S.A., 85:7448-7451 (1988)), etc.

While antisense nucleotides complementary to the coding region sequence of the invention could be used, those complementary to the transcribed untranslated region are most preferred.

Potential antagonists according to the invention also include catalytic RNA, or a ribozyme (See, e.g., PCT International Publication WO 90/11364, published Oct. 4, 1990; Sarver et al, Science, 247:1222-1225 (1990). While ribozymes that cleave mRNA at site specific recognition sequences can be used to destroy mRNAs corresponding to the polynucleotides of the invention, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature, 334:585-591 (1988). There are numerous potential hammerhead ribozyme cleavage sites within each nucleotide sequence disclosed in the Sequence Listing. Preferably, the ribozyme is engineered so that the cleavage recognition site is located near the 5′ end of the mRNA corresponding to the polynucleotides of the invention; i.e., to increase efficiency and minimize the intracellular accumulation of non-functional mRNA transcripts.

As in the antisense approach, the ribozymes of the invention can be composed of modified oligonucleotides (e.g. for improved stability, targeting, etc.) and should be delivered to cells which express the polynucleotides of the invention in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities' of the ribozyme to destroy endogenous messages and inhibit translation. Since ribozymes unlike antisense molecules, are catalytic, a lower intracellular concentration is required for efficiency.

Antagonist/agonist compounds may be employed to inhibit the cell growth and proliferation effects of the polypeptides of the present invention on neoplastic cells and tissues, i.e. stimulation of angiogenesis of tumors, and, therefore, retard or prevent abnormal cellular growth and proliferation, for example, in tumor formation or growth.

The antagonist/agonist may also be employed to prevent hyper-vascular diseases, and prevent the proliferation of epithelial lens cells after extracapsular cataract surgery. Prevention of the mitogenic activity of the polypeptides of the present invention may also be desirous in cases such as restenosis after balloon angioplasty.

The antagonist/agonist may also be employed to prevent the growth of scar tissue during wound healing.

The antagonist/agonist may also be employed to treat, prevent, and/or diagnose the diseases described herein.

Thus, the invention provides a method of treating or preventing diseases, disorders, and/or conditions, including but not limited to the diseases, disorders, and/or conditions listed throughout this application, associated with overexpression of a polynucleotide of the present invention by administering to a patient (a) an antisense molecule directed to the polynucleotide of the present invention, and/or (b) a ribozyme directed to the polynucleotide of the present invention.

### Other Preferred Embodiments

Other preferred embodiments of the claimed invention include an isolated nucleic acid molecule comprising a nucleotide sequence containing one or more polymorphic positions and is at least about 20, 25, 30, 35, 40, 45, or 50 contiguous nucleotides and is derived from a nucleotide sequence defined in Table I.

Also preferred is a nucleic acid molecule wherein said sequence of contiguous nucleotides is included in the nucleotide sequence of SEQ ID NO:X in the range of positions beginning with the nucleotide at about the position of the “5′ NT of Start Codon of ORF” and ending with the nucleotide at about the position of the “3′ NT of ORF” as defined for SEQ ID NO:X in Table I.

Also preferred is an isolated nucleic acid molecule comprising a nucleotide sequence containing at least one or more polymorphic positions and is at least about 150 contiguous nucleotides in the nucleotide sequence of SEQ ID NO:X.

Further preferred is an isolated nucleic acid molecule comprising a nucleotide sequence containing at least one or more polymorphic positions and is at least about 500 contiguous nucleotides in the nucleotide sequence of SEQ ID NO:X.

A further preferred embodiment is a nucleic acid molecule comprising a nucleotide sequence containing one or more polymorphic positions and corresponds to, or is derived from, SEQ ID NO:X beginning with the nucleotide at about the position of the “5′ NT of ORF” and ending with the nucleotide at about the position of the “3′ NT of ORF” as defined for SEQ ID NO:X in Table I.

A further preferred embodiment is an isolated nucleic acid molecule comprising a nucleotide sequence containing one or more polymorphic positions and correponds to, or is derived from, the complete nucleotide sequence of SEQ ID NO:X.

Also preferred is an isolated nucleic acid molecule which hybridizes under stringent hybridization conditions to a nucleic acid molecule, wherein said nucleic acid molecule which hybridizes does not hybridize under stringent hybridization conditions to a nucleic acid molecule having a nucleotide sequence consisting of only A residues or of only T residues.

Also preferred is a composition of matter comprising a DNA molecule which comprises a cDNA clone identified by a SNP_ID Identifier in Table I.

Also preferred is an isolated nucleic acid molecule comprising a nucleotide sequence containing at least one or more polymorphic positions and is at least 20 contiguous nucleotides in the nucleotide sequence of a cDNA clone identified by a SNP_ID Identifier in Table I.

Also preferred is an isolated nucleic acid molecule, wherein said sequence of at least 20 contiguous nucleotides is included in the nucleotide sequence of the complete open reading frame sequence encoded by said cDNA clone.

A further preferred embodiment is a method for detecting in a biological sample a nucleic acid molecule comprising a nucleotide sequence containing at least one or more polymorphic positions and is at least 20 contiguous nucleotides in a sequence selected from the group consisting of: a nucleotide sequence of SEQ ID NO:X wherein X is any integer as defined in Table I; which method comprises a step of comparing a nucleotide sequence of at least one nucleic acid molecule in said sample with a sequence selected from said group and determining whether the sequence of said nucleic acid molecule in said sample contains one or more polymorphic positions relative to said selected sequence.

Also preferred is the above method wherein said step of comparing sequences comprises determining the extent of nucleic acid hybridization between nucleic acid molecules in said sample and a nucleic acid molecule comprising said sequence selected from said group. Similarly, also preferred is the above method wherein said step of comparing sequences is performed by comparing the nucleotide sequence determined from a nucleic acid molecule in said sample with said sequence selected from said group. The nucleic acid molecules can comprise DNA molecules or RNA molecules.

A further preferred embodiment is a method for identifying the species, tissue or cell type of a biological sample which method comprises a step of detecting nucleic acid molecules in said sample, if any, comprising a nucleotide sequence containing one or more polymorphic positions and corresponds to, or is derived from, a sequence that is at least 20 contiguous nucleotides in a sequence selected from the group consisting of: a nucleotide sequence of SEQ ID NO:X wherein X is any integer as defined in Table I; and a nucleotide sequence encoded by a cDNA clone identified a SNP_ID Identifier in Table I.

The method for identifying the species, tissue or cell type of a biological sample can comprise a step of detecting nucleic acid molecules comprising a nucleotide sequence in a panel of at least two nucleotide sequences, wherein at least one sequence in said panel contains one or more polymorphic positions to a sequence of at least 20 contiguous nucleotides in a sequence selected from said group.

Also preferred is a method for diagnosing in a subject a pathological condition associated with abnormal structure or expression of a gene encoding a protein identified in Table I, which method comprises a step of detecting in a biological sample obtained from said subject nucleic acid molecules, if any, comprising a nucleotide sequence that contains one or more polymorphic positions and corresponds to, or is derived from, a sequence that is at least 20 contiguous nucleotides in a sequence selected from the group consisting of: a nucleotide sequence of SEQ ID NO:X wherein X is any integer as defined in Table I; and a nucleotide sequence encoded by a cDNA clone identified by a SNP_ID Identifier in Table I.

The method for diagnosing a pathological condition can comprise a step of detecting nucleic acid molecules comprising a nucleotide sequence in a panel of at least two nucleotide sequences, wherein at least one sequence in said panel contains one or more polymorphic positions and is derived from, or corresponds to, a sequence that is at least 20 contiguous nucleotides in a sequence selected from said group.

Also preferred is a composition of matter comprising isolated nucleic acid molecules wherein the nucleotide sequences of said nucleic acid molecules comprise a panel of at least two nucleotide sequences, wherein at least one sequence in said panel contains one or more polymorphic positions and is derived from, or corresponds to, a sequence that is at least 20 contiguous nucleotides in a sequence selected from the group consisting of: a nucleotide sequence of SEQ ID NO:X wherein X is any integer as defined in Table I; and a nucleotide sequence encoded by a cDNA clone identified by a SNP_ID Identifier in Table I. The nucleic acid molecules can comprise DNA molecules or RNA molecules.

Also preferred is an isolated polypeptide comprising an amino acid sequence containing one or more polymorphic positions and is derived from, or corresponds to, a sequence that is at least about 10 contiguous amino acids in the amino acid sequence of SEQ ID NO:Y wherein Y is any integer as defined in Table I.

Also preferred is an isolated polypeptide comprising an amino acid sequence containing one or more polymorphic positions and is derived from, or corresponds to, a sequence that is at least about 10 contiguous amino acids and is encoded by a nucleotide sequence provided in Table I.

Also preferred is an isolated polypeptide comprising an amino acid sequence containing one or more polymorphic positions and is derived from, or corresponds to, a sequence that is at least about 30 contiguous amino acids in the amino acid sequence of SEQ ID NO:Y.

Further preferred is an isolated polypeptide comprising an amino acid sequence containing one or more polymorphic positions and is derived from, or corresponds to, a sequence that is at least about 100 contiguous amino acids in the amino acid sequence of SEQ ID NO:Y.

Further preferred is an isolated polypeptide comprising an amino acid sequence containing one or more polymorphic positions and is derived from, or corresponds to, the complete amino acid sequence of SEQ ID NO:Y.

Also preferred is a polypeptide wherein said sequence of contiguous amino acids is included in the amino acid sequence of the protein encoded by a cDNA clone identified by a SNP_ID Identifier in Table I.

Also preferred is an isolated polypeptide comprising an amino acid sequence containing one or more polymorphic positions and is derived from, or corresponds to, a sequence that is at least about 30 contiguous amino acids in the amino acid sequence of the protein encoded by a cDNA clone identified by a SNP_ID Identifier in Table I.

Also preferred is an isolated polypeptide comprising an amino acid sequence containing one or more polymorphic positions and is derived from, or corresponds to, a sequence that is at least about 100 contiguous amino acids in the amino acid sequence of the protein encoded by a cDNA clone identified by a SNP_ID Identifier in Table I.

Also preferred is an isolated polypeptide comprising an amino acid sequence containing one or more polymorphic positions and is derived from, or corresponds to, the amino acid sequence of the protein encoded by a cDNA clone identified by a SNP_ID Identifier in Table I.

Further preferred is an isolated antibody which binds specifically to a polypeptide comprising an amino acid sequence containing one or more polymorphic positions and is derived from, or corresponds to, a sequence that is at least 10 contiguous amino acids in a sequence selected from the group consisting of: an amino acid sequence of SEQ ID NO:Y wherein Y is any integer as defined in Table I; and a complete amino acid sequence of a protein encoded by a cDNA clone identified by a cDNA Clone Identifier in Table I, and/or an SNP_ID Identifier in Table III, or IV.

Further preferred is a method for detecting in a biological sample a polypeptide comprising an amino acid sequence containing one or more polymorphic positions and is derived from, or corresponds to, a sequence that is at least 10 contiguous amino acids in a sequence selected from the group consisting of: an amino acid sequence of SEQ ID NO:Y wherein Y is any integer as defined in Table I, and/or in Table VI; and a complete amino acid sequence of a protein encoded by a cDNA clone identified by a SNP_ID Identifier in Table I; which method comprises a step of comparing an amino acid sequence of at least one polypeptide molecule in said sample with a sequence selected from said group and determining whether the sequence of said polypeptide molecule in said sample containing one or more polymorphic positions and is derived from, or corresponds to, a sequence that is at least 10 contiguous amino acids.

Also preferred is the above method wherein said step of comparing an amino acid sequence of at least one polypeptide molecule in said sample with a sequence selected from said group comprises determining the extent of specific binding of polypeptides in said sample to an antibody which binds specifically to a polypeptide comprising an amino acid sequence containing one or more polymorphic positions and is derived from, or corresponds to, a sequence that is at least 10 contiguous amino acids in a sequence selected from the group consisting of: an amino acid sequence of SEQ ID NO:Y wherein Y is any integer as defined in Table I; and a complete amino acid sequence of a protein encoded by a cDNA clone identified by a SNP_ID Identifier in Table I.

Also preferred is the above method wherein said step of comparing sequences is performed by comparing the amino acid sequence determined from a polypeptide molecule in said sample with said sequence selected from said group.

Also preferred is a method for identifying the species, tissue or cell type of a biological sample which method comprises a step of detecting polypeptide molecules in said sample, if any, comprising an amino acid sequence containing one or more polymorphic positions and is derived from, or corresponds to, a sequence that is at least 10 contiguous amino acids in a sequence selected from the group consisting of: an amino acid sequence of SEQ ID NO:Y wherein Y is any integer as defined in Table I; and a complete amino acid sequence of a protein encoded by a cDNA clone identified by a SNP_ID Identifier in Table I.

Also preferred is the above method for identifying the species, tissue or cell type of a biological sample, which method comprises a step of detecting polypeptide molecules comprising an amino acid sequence in a panel of at least two amino acid sequences, wherein at least one sequence in said panel containing one or more polymorphic positions and is derived from, or corresponds to, a sequence that is at least 10 contiguous amino acids in a sequence selected from the above group.

Also preferred is a method for diagnosing a pathological condition associated with an organism with abnormal structure or expression of a gene encoding a protein identified in Table I, which method comprises a step of detecting in a biological sample obtained from said subject polypeptide molecules comprising an amino acid sequence in a panel of at least two amino acid sequences, wherein at least one sequence in said panel containing one or more polymorphic positions and is derived from, or corresponds to, a sequence that is at least 10 contiguous amino acids in a sequence selected from the group consisting of: an amino acid sequence of SEQ ID NO:Y wherein Y is any integer as defined in Table I; and a complete amino acid sequence of a protein encoded by a cDNA clone identified by a SNP_ID Identifier in Table I.

In any of these methods, the step of detecting said polypeptide molecules includes using an antibody.

Also preferred is an isolated nucleic acid molecule, wherein said nucleotide sequence encoding a polypeptide has been optimized for expression of said polypeptide in a prokaryotic host.

Also preferred is an isolated nucleic acid molecule, wherein said polypeptide comprises an amino acid sequence selected from the group consisting of: an amino acid sequence of SEQ ID NO:Y wherein Y is any integer as defined in Table I, and/or in Table VI; and a complete amino acid sequence of a protein encoded by a cDNA clone identified by a SNP_ID Identifier in Table I.

Further preferred is a method of making a recombinant vector comprising inserting any of the above isolated nucleic acid molecule(s) into a vector. Also preferred is the recombinant vector produced by this method. Also preferred is a method of making a recombinant host cell comprising introducing the vector into a host cell, as well as the recombinant host cell produced by this method.

Also preferred is a method of making an isolated polypeptide comprising culturing this recombinant host cell under conditions such that said polypeptide is expressed and recovering said polypeptide. Also preferred is this method of making an isolated polypeptide, wherein said recombinant host cell is a eukaryotic cell and said polypeptide is a protein comprising an amino acid sequence selected from the group consisting of: an amino acid sequence of SEQ ID NO:Y wherein Y is an integer set forth in Table I and said position of the “Total AA of ORF” of SEQ ID NO:Y is defined in Table I; and an amino acid sequence of a protein encoded by a cDNA clone identified by a SNP_ID identifier in Table I. The isolated polypeptide produced by this method is also preferred.

Also preferred is a method of treatment of an individual in need of an increased level of a protein activity, which method comprises administering to such an individual a pharmaceutical composition comprising an amount of an isolated polypeptide, polynucleotide, or antibody of the claimed invention effective to increase the level of said protein activity in said individual.

The following Examples are offered for the purpose of illustrating the present invention and are not to be construed to limit the scope of this invention. The teachings of all references cited herein are hereby incorporated herein by reference. REFERENCES

Agostoni A, C. M., Cugno M, Zingale L C, Gioffre D, Nussberger J (1999). “Angioedema due to angiotensin-converting enzyme inhibitors.” Immunopharmacology Oct. 15, 1999;44 (1-2):21-5 44 (1-2): 21-25.

Altschul S F, G. W., Miller W, Myers E W, Lipman D J (1990). “Basic local alignment search tool.” J Mol Biol 215 (3): 403-410.

Anderson M W, d. R. (1990). “Studies of the mechanism of angiotensin-converting enzyme (ACE) inhibitor-associated angioedema: the effect of an ACE inhibitor on cutaneous responses to bradykinin, codeine, and histamine.” J Allergy Clin Immunol 85 (5): 856-858.

Barnes, P. J. (1997). Bradykinin. Asthma. P. J. Barnes, M. M. Grunstein, A. R. Leff and A. J. Woolcock. Philadelphia, New York, Lippincott-Raven. 1: 577-584.

Brown N J, R. W., Snowden M, Griffin M R (1996). “Black Americans have an increased rate of angiotensin converting enzyme inhibitor-associated angioedema.” Clin Pharmacol Ther 60 (1): 8-13.

Brown N J, S. M., Griffin M R (1997). “Recurrent angiotensin-converting enzyme inhibitor—associated angioedema.” JAMA 278 (3): 232-233.

Brown N J, V. D. (1998). “Angiotensin-converting enzyme inhibitors.” Circulation 97 (14): 1411-1420.

Chen X, L. L., Kwok P Y. (1999). “Fluorescence polarization in homogeneous nucleic acid analysis.” Genome Res. May 9, 1999; (5):492-8. 9 (5): 492-498.

Coats, A. (2000). “Omapatrilat—the ups and downs of an exciting but complicated new drug.” Int J Cardiol 74 (1): 1-3.

Cormack, B. (2000). Directed Mutagenesis Using the Polymerase Chain Reaction. Current Protocols in Molecular Biology, John Wiley & Sons, Inc. Supplement 37: 8.5.1-8.5.10.

Ebo D G, S. W. (2000). “Hereditary angioneurotic edema: review of the literature.” Acta Clin Belg January-February 2000;55 (1):22-9 55 (1): 22-29.

Lennon G, A. C., Polymeropoulos M, Soares M B (1996). “The I.M.A.G.E. Consortium: an integrated molecular analysis of genomes and their expression.” Genomics 33 (1): 151-152.

Marceau, F., J. Hess, et al. (1998). “The B1 receptors for kinins.” Pharmacol Rev 50 (3): 357-386.

Marceau F, B. D. (1998). “Kinin receptors.” Clin Rev Allergy Immunol 16 (4): 385-401.

Marceau F, L. J., Saint-Jacques E, Bachvarov D R. (1997). “The kinin B1 receptor: an inducible G protein coupled receptor.” Can J Physiol Pharmacol 75 (6): 725-730.

Marceau F, L. J., Bouthillier J, Bachvarova M, Houle S, Bachvarov D R (1999). “Effect of endogenous kinins, prostanoids, and NO on kinin B1 and B2 receptor expression in the rabbit.” Am J Physiol 277 (6 Pt 2): R1568-R1578.

Nickerson D A, T. V., Taylor S L. (1997). “PolyPhred: automating the detection and genotyping of single nucleotide substitutions using fluorescence-based resequencing.” Nucleic Acids Res 25 (14): 2745-2751.

Nussberger, J., M. Cugno, et al. (1998). “Plasma bradykinin in angio-edema.” Lancet 351: 1693-1697.

Nussberger J, C. M., Cicardi M, Agostoni A. (1999). “Local bradykinin generation in hereditary angioedema.” J Allergy Clin Immunol. December 1999;104 (6):1321-2 104 (6): 1321-1322.

Rieder M J, T. S., Clark A G, Nickerson D A (1999). “Sequence variation in the human angiotensin converting enzyme.” Nat Genet 22 (1): 59-62.

Robl J A, S. C., Stevenson J, Ryono D E, Simpkins L M, Cimarusti M P, Dejneka T, Slusarchyk W A, Chao S, Stratton L, Misra R N, Bednarz M S, Asaad M M, Cheung H S, Abboa-Offei B E, Smith P L, Mathers P D, Fox M, Schaeffer T R, Seymour A A, Trippodo N C (1997). “Dual metalloprotease inhibitors: mercaptoacetyl-based fused heterocyclic dipeptide mimetics as inhibitors of angiotensin-converting enzyme and neutral endopeptidase.” J Med Chem 40 (11): 1570-1577.

Rozen S, S. H. (2000). “Primer3 on the WWW for general users and for biologist programmers.” Methods Mol Biol 132: 365-386.

Sambrook, J., E. Fritsch, et al. (1989). Molecular Cloning, Cold Spring Harbor Laboratory Press.

Schiller P I, M. S., Haefeli W E, Schlienger R G, Bircher A J (1997). “Angiotensin-converting enzyme inhibitor-induced angioedema: late onset, irregular course, and potential role of triggers.” Allergy 52 (4): 432-435.

Strader, C., T. Fong, et al. (1995). “The family of G-protein-coupled receptors.” FASEB J 9 (9): 745-754.

Tosi, M. (1998). “Molecular genetics of C1 inhibitor.” Immunobiology 199 (2): 358-365.

van Rijnsoever E W, K.-Z. W., Feenstra J. (1998). “Angioneurotic edema attributed to the use of losartan.” Arch Intern Med. Oct. 12, 1998;158 (18):2063-5. 158 (18): 2063-2065.

### EXAMPLES

### Example 1

Method of Discovering the Single Nucleotide Polymorphisms (SNPs) of the Present Invention

Candidate genes for SNP discovery were chosen from the bradykinin pathway based upon their involvement in the following pathway processes: i.) Generation of bradykinin and related peptides: C1 esterase inhibitor, kininogen, tissue and plasma kallikreins; ii.) Degradation of bradykinin, and related peptides: ACE, NEP, aminopeptidase P, carboxypeptidases M, N, and U; and iii.) Bradykinin signal transduction: B1 and B2 bradykinin receptors, NK1 tachykinin receptor. Additional genes were chosen for SNP discovery based upon the identification of other SNPs within those genes from prior SNP discovery studies (see co-pending patent applications U.S. Ser. No. 10/005,956 and U.S. Ser. No. 60/353,790); which are hereby incorporated herein by reference in their entirety). Specifically, the following genes were analyzed for the presence of potential SNPs: C1, S subcomponent (HGNC ID: C1S), alanyl aminopeptidase (HGNC_ID: ANPEP), meprin A, beta (HGNC_ID: MEP1B), Aminopeptidase P-like (HGNC_ID: XPNPEPL), Tissue kallikrein (HGNC_ID: KLK1), Aminopeptidase P (membrane bound) (HGNC_ID: XPNPEP2), and Soluble guanylate cyclase 1, alpha-2 subunit (HGNC_ID: GUCY1A2).

SNP discovery was based on comparative DNA sequencing of PCR products derived from genomic DNA from multiple individuals. All the genomic DNA samples were obtained from patients enrolled in a Bristol-Myers Squibb Company omapatrilat clinical study (see Table VIII). PCR amplicons were designed to cover the entire coding region of the exons using the Primer3 program (Rozen S 2000). Exon-intron structure of candidate genes and intron sequences were obtained by blastn search of Genbank cDNA sequences against the human genome draft sequences. The sizes of these PCR amplicons varied according to the exon-intron structure. All the samples amplified from genomic DNA (20 ng) in reactions (50 ul) containing 10 mM Tris-Cl pH 8.3, 50 mM KCl, 2.5 mM MgCl₂, 150 uM dNTPs, 3 uM PCR primers, and 3.75 U TaqGold DNA polymerase (PE Biosystems).

PCR was performed in MJ Research Tetrad machines under a cycling condition of 94 degrees 10 min, 30 cycles of 94 degrees 30 sec, 60 degrees 30 sec, and 72 degrees 30 sec, followed by 72 degrees 7 min. PCR products were purified using QIAquick PCR purification kit (Qiagen), and were sequenced by the dye-terminator method using PRISM 3700 automated DNA sequencer (Applied Biosystems, Foster City, Calif.) following the manufacturer's instruction outlined in the Owner's Manual (which is hereby incorporated herein by reference in its entirety). Sequencing results were analyzed for the presence of polymorphisms using PolyPhred software(Nickerson D A 1997; Rieder M J 1999). All the sequence traces of potential polymorphisms were visually inspected to confirm the presence of SNPs.

DNA sequences of PCR primers and sequencing primers used for SNP discovery are provided in Tables IV and V, respectfully.

Alternative methods for identifying SNPs of the present invention are known in the art. One such method involves resequencing of target sequences from individuals of diverse ethnic and geographic backgrounds by hybridization to probes immobilized to microfabricated arrays. The strategy and principles for the design and use of such arrays are generally described in WO 95/11995.

A typical probe array used in such as analysis would have two groups of four sets of probes that respectively tile both strands of a reference sequence. A first probe set comprises a plurality of probes exhibiting perfect complementarily with one of the reference sequences. Each probe in the first probe set has an interrogation position that corresponds to a nucleotide in the reference sequence. That is, the interrogation position is aligned with the corresponding nucleotide in the reference sequence, when the probe and reference sequence are aligned to maximize complementarily between the two. For each probe in the first set, there are three corresponding probes from three additional probe sets. Thus, there are four probes corresponding to each nucleotide in the reference sequence. The probes from the three additional probe sets would be identical to the corresponding probe from the first probe set except at the interrogation position, which occurs in the same position in each of the four corresponding probes from the four probe sets, and is occupied by a different nucleotide in the four probe sets. In the present analysis, probes were nucleotides long. Arrays tiled for multiple different references sequences were included on the same substrate.

Publicly available sequences for a given gene can be assembled into Gap4 (http://www.biozentrum.unibas.ch/-biocomp/staden/Overview.html). PCR primers covering each exon, could be designed, for example, using Primer 3 (httP://www-genome.wi.mit.edu/cgi-bin/primer/primer3.cgi). Primers would not be designed in regions where there are sequence discrepancies between reads. Genomic DNA could be amplified from at least two individuals using 2.5 pmol each primer, 1.5 mM MgCl2, 100˜M dNTPs, 0.75˜M AmpliTaq GOLD polymerase, and about 19 ng DNA in a 15 ul reaction. Reactions could be assembled using a PACKARD MultiPROBE robotic pipetting station and then put in MJ 96-well tetrad thermocyclers (96° C. for minutes, followed by cycles of 96° C. for seconds, 59° C. for 2 minutes, and 72° C. for 2 minutes). A subset of the PCR assays for each individual could then be run on 3% NuSieve gels in 0.5× TBE to confirm that the reaction worked.

For a given DNA, 5 ul (about 50 ng) of each PCR or RT -PCR product could be pooled (Final volume=150-200 ul). The products can be purified using QiaQuick PCR purification from Qiagen. The samples would then be eluted once in 35 ul sterile water and 4 ul 1OX One-Phor-All buffer (Pharmacia). The pooled samples are then digested with 0.2 u DNaseI (Promega) for 10 minutes at 37° C. and then labeled with 0.5 nmols biotin-N6-ddATP and 15 u Terminal Transferase (GibcoBRL Life Technology) for 60 minutes at 37° C. Both fragmentation and labeling reactions could be terminated by incubating the pooled sample for 15 minutes at 100° C.

Low-density DNA chips {Affymetrix, CA) may be hybridized following the manufacturer's instructions. Briefly, the hybridization cocktail consisted of 3M TMACl, mM Tris pH 7.8, 0.01% Triton X-100, 100 mg/ml herring sperm DNA {Gibco BRL), 200 pM control biotin-labeled oligo. The processed PCR products are then denatured for 7 minutes at 100° C. and then added to prewarmed {37° C.) hybridization solution. The chips are hybridized overnight at 44° C. Chips are washed in 1× SSPET and 6× SSPET followed by staining with 2 ug/ml SARPE and 0.5 mg/ml acetylated BSA in 200 ul of 6× SSPET for 8 minutes at room temperature. Chips are scanned using a Molecular Dynamics scanner.

Chip image files may be analyzed using Ulysses {Affymetrix, CA) which uses four algorithms to identify potential polymorphisms. Candidate polymorphisms may be visually inspected and assigned a confidence value: where high confidence candidates display all three genotypes, while likely candidates show only two genotypes {homozygous for reference sequence and heterozygous for reference and variant). Some of the candidate polymorphisms may be confirmed by ABI sequencing. Identified polymorphisms could then be compared to several databases to determine if they are novel.

### Example 2

Method of Determining the Allele Frequency for Each SNP of the Present Invention

Allele frequencies of these polymorphisms were determined by genotyping Caucasian, and African American DNA samples obtained from a Bristol-Myers Squibb omapatrilat clinical study using the FP-TDI assay (Chen X 1999). The ethnicity for each of the DNA samples utilized for the present invention are provided in Table VIII. Automated genotyping calls were made with an allele calling software developed by Joel Hirschorn (Whitehead Institute/MIT Center for Genome Research, personal communication).

Briefly, the no template controls (NTCs) were labeled accordingly in column C. The appropriate cells were completed in column L indicating whether REF (homozygous ROX) or VAR (homozygous TAMRA) are expected to be rare genotypes (<10% of all samples)—the latter is important in helping the program to identify rare homozygotes. The number of 96 well plates genotyped in cell P2 are noted (generally between 0.5 and 4)—the program works best if this is accurate. No more than 384 samples can be analyzed at a time. The pairs of mP values from the LJL were pasted into columns E and F; making sure there were no residual data was left at the bottom fewer than 384 data points are provided. The DNA names were provided in columns A, B or C; column I will be a concatenation of columns A, B and C. In addition, the well numbers for each sample were also provided in column D.

With the above information provided, the program should automatically cluster the points and identify genotypes. The program works by converting the mP values into polar coordinates (distance from origin and angle from origin) with the angle being on a scale from 0 to 2; heterozygotes are placed as close to 1 as possible.

The cutoff values in columns L and M may be adjusted as desired.

Expert parameters: The most important parameters are the maximum angle for REF and minimum angle for VAR. These parameters may need to be changed in a particularly skewed assay which may be observed when an REF or VAR cluster is close to an angle of 1 and has called as a failed or HETs.

Other parameters are low and high cutoffs that are used to determine which points are considered for the determination of edges of the clusters. With small numbers of data points, the high cutoff may need to be increased (to 500 or so). This may be the right thing to do for every assay, but certainly when the program fails to identify a small cluster with high signal.

NTC TAMRA and ROX indicate the position of the no template control or failed samples as estimated by the computer algorithm.

No signal=mP<is the threshold below which points are automatically considered failures. “Throw out points with signal above” is the TAMRA or ROX mP value above which points are considered failures. The latter may occasionally need to be adjusted from 250 to 300, but caveat emptor for assays with signals >250. ‘Lump’ or ‘split’ describes a subtle difference in the way points are grouped into clusters. Lump generally is better. ‘HETs expected’ in the rare case where only homozygotes of either class are expected (e.g. a study of X chromosome SNPs in males), change this to “N”.

Notes on method of clustering: The origin is defined by the NTCs or other low signal points (the position of the origin is shown as “NTC TAMRA” and “NTC ROX”); the points with very low or high signal are not considered initially. The program finds the point farthest from the origin and calls that a HET; the ROX/TAMRA ratio is calculated from this point, placing the heterozygotes at 45 degrees from the origin (an angle of “1”). The angles from the origin are calculated (the scale ranges from 0 to 2) and used to define clusters. A histogram of angles is generated. The cluster boundaries are defined by an algorithm that takes into account the shape of the histogram. The homozygote clusters are defined as the leftmost and rightmost big clusters (unless the allele is specified as being rare, in which case the cluster need not be big). The heterozygote is the biggest cluster in between the REF and VAR. If there are two equal clusters, the one best-separated from REF and VAR is called HET. All other clusters are failed. Some fine tuning is applied to lump in scattered points on the edges of the clusters (if “Lump” is selected). The boundaries of the clusters are “Angles” in column L.

Once the clusters are defined, the interquartile distance of signal intensity is defined for each cluster. Points falling more than 3 or 4 interquartiles from the mean are excluded. (These are the “Signal cutoffs” in column M).

The allelic frequency of a subset of the SNPs of the present invention are provided in Table XXXX.

The invention encompasses additional methods of determining the allelic frequency of the SNPs of the present invention. Such methods may be known in the art, some of which are described elsewhere herein.

### Example 3

Method of Genotyping each SNP of the Present Invention

### Genomic DNA Preparation

Genomic DNA samples for genotyping may be prepared using the Purigene™ DNA extraction kit from Gentra Systems (http://www.gentra.com). After preparation, DNA samples may be diluted to a 2 ng/ul working concentration with TE buffer (10 mM Tris-Cl, pH 8.0, 0.1 mM EDTA, pH 8.0) and stored in 1 ml 96 deep well plates (VWR) at −20 degrees until use.

Samples for genomic DNA preparations may be obtained from patients participating in a Bristol-Myers Squibb (BMS) omapatrilat clinical study, or from other sources known in the art or otherwise described herein.

### Genotyping

The SNP genotyping reactions may be performed using the SNPStream™ system (Orchid Biosience, Princeton, N.J.) based on genetic bit analysis (Nikiforov, T. et al, Nucleic Acids Res 22, 4167-4175 (1994)).

The regions including polymorphic sites may be amplified by the polymerase chain reaction (PCR) using a pair of primers (OPERON Technologies), one of which is phosphorothioated. 6 ul PCR cocktail containing 1.0 ng/ul genomic DNA, 200 uM dNTPs, 0.5 uM forward PCR primer, 0.5 uM reverse PCR primer (phosphorothioated), 0.05 u/ul Platinum Taq DNA polymerase (LifeTechnologies), and 1.5 mM MgCl₂. The PCR primer pairs that may be used for genotyping analysis are provided in Table VI under the headings ‘ORCHID_FORWARD (SEQ ID Nos: 1240 thru 1250) and ‘ORCHID_REVERSE’ (SEQ ID Nos: 1251 thru 1261). The PCR reaction was set up in 384-well plates (MJ Research) using a MiniTrak liquid handling station (Packard Bioscience). The PCR primer sequences may be selected from those provided in Table VI herein, or any other primer as may otherwise be required. PCR thermocycling may be performed under the following conditions in a MJ Research Tetrad machine: stepl, 95 degrees for 2 min; step 2, 94 degrees for 30 min; step 3, 55 degrees for 2 min; step 4, 72 degrees for 30 sec; step 5, go back to step 2 for an additional 39 cycles; step 6, 72 degrees for 1 min; and step 7, 12 degrees indefinitely)

After thermocycling, the amplified samples may be placed in the SNPStream™ (Orchid Bioscience) machine, and automated genetic bit analysis (GBA) (Nikiforov, T. et al,supra) reaction can then be performed. The first step of this reaction is degradation of one of the strands of the PCR products by T7 gene 6 exonuclease to make them single-stranded. The strand containing the phosphorothioated primer are resistant to T7 gene 6 nuclease, and were not degraded by this enzyme. After digestion, the single-stranded PCR products are subjected to an annealing step whereby the single stranded PCR products are annealed to the GBA primer on a solid phase, and then subjected to the GBA reaction (single base extension) using dideoxy-NTPs labeled with biotin or fluorescein. The GBA primers used for single base extension are provided in Table IX under the heading ‘ORCHID_SNPIT’ (SEQ ID Nos:X),. Polynucleotide bases represented by an “N” in Table IX represent bases that are substituted with a C3 linker (C3 spacer phosphoramidite) during synthesis of the primer. Such linkers may be obtained from Research Genetics, and Sigma-Genosys, for example. The ‘ORCHID_SNPIT’ primers are obtained from Operon. Incorporation of these dideoxynucleotides into a GBA primer are detected by a two color ELISA assay using anti-fluorescein alkaline phosphatase conjugate and anti-biotin horseradish peroxidase. Automated genotype calls are made by GenoPak software (Orchid Bioscience), before manual correction of automated calls are done upon inspection of the resulting allelogram of each SNP.

### Example 4

Alternative Method of Genotyping each SNP of the Present Invention

In addition to the method of genotyping described in Example 3, the skilled artisan could determine the genotype of the polymorphisms of the present invention using the below described alternative method. This method is referred to as the “GBS method” herein and may be performed as described in conjunction with the teachings described elsewhere herein.

Briefly, the direct analysis of the sequence of the polymorphisms of the present invention can be accomplished by DNA sequencing of PCR products corresponding to the same. PCR amplicons are designed to be in close proximity to the polymorphisms of the present invention using the Primer3 program. The M13_SEQUENCE1 “TGTAAAACGACGGCCAGT (SEQ ID NO:1213)” is prepended to each forward PCR primer. The M13_SEQUENCE2 “CAGGAAACAGCTATGACC (SEQ ID NO: 1214)” is prepended to each reverse PCR primer. The specific GBS forward PCR primers for each SNP of the present invention are provided in Table VII under the “GBS_LEFT” column (SEQ ID NO: 897 thru 1043). The specific GBS reverse PCR primers for each SNP of the present invention are provided in Table VII under the “GBS_RIGHT” column (SEQ ID NO: 1044 thru 1190).

PCR amplification may be performed on genomic DNA samples amplified from (20 ng) in reactions (50 ul) containing 10 mM Tris-Cl pH 8.3, 50 mM KCl, 2.5 mM MgCl₂, 150 uM dNTPs, 3 uM PCR primers, and 3.75 U TaqGold DNA polymerase (PE Biosystems). PCR was performed in MJ Research Tetrad machines under a cycling condition of 94 degrees 10 min, 30 cycles of 94 degrees 30 sec, 60 degrees 30sec, and 72 degrees 30 sec, followed by 72 degrees 7 min. PCR products were purified using QIAquick PCR purification kit (Qiagen), and were sequenced by the dye-terminator method using PRISM 3700 automated DNA sequencer (Applied Biosystems, Foster City, Calif.) following the manufacturer's instruction outlined in the Owner's Manual (which is hereby incorporated herein by reference in its entirety).essentially the same as described in herein.

PCR products are sequenced by the dye-terminator method using the M13_SEQUENCE1 (SEQ ID NO:1213) and M13_SEQUENCE2 (SEQ ID NO:1214) primers above. The genotype can be determined by analysis of the sequencing results at the polymorphic position.

### Example5

Statistical Analysis of the Association Between the Angioedema Phenotype and the SNPs of the Present Invention

The association between angioedema and the single nucleotide polymorphisms of the present invention may be investigated by applying statistical analysis to the results of the genotyping assays described elsewhere herein. The central hypothesis of this analysis is that a predisposition to develop angioedema may be conferred by specific genomic factors. The analysis attempted to identify one or more of these factors in DNA samples from index cases and matched control subjects who were exposed to omapatrilat ([4S-[4.alpha. (R*), 7.alpha., 10a.beta.]]-octahydro-4-[ (2-mercapto-1-oxo-3-phenylpropyl)amino]-5-oxo-7H-pyrido[2,1-b] [1,3]thiazepine-7-carboxylic acid) in a Bristol-Myers Squibb (BMS) omapatrilat clinical study.

### Methods

Sample. Investigators in the BMS omapatrilat clinical trial diagnosed angioedema in some subjects. Head and neck edema, which shares some clinical features with angioedema, for example, lip swelling, was also identified in some subjects. One subject experienced angioedema and head and neck edema. For the purposes of statistical analysis, this individual was considered only as an angioedema case. In this study, “head and neck edema” is referred to as an “angioedema-like event”.

Prior to initiating this analysis, listings of index cases and matched controls were generated from subjects that participated in a Bristol-Myers Squibb omapatrilat clinical program. These listings pre-specified the population of subjects that were to be enrolled at the investigative sites. Case subjects who were previously exposed to omapatrilat and experienced angioedema or angioedema-like events were matched with control subjects who were exposed to omapatrilat but did not experience angioedema or angioedema-like events. Matched controls were identified for each index case based on nationality, race, gender, and starting dose of omapatrilat. Matching did not include other potential angioedema risk factors such as dose escalation, age, tobacco use and allergy history. To reduce the total number of sites to a manageable level, controls for Non-Black index cases were selected based on the matching criteria from those sites with an index case. Controls for Black index cases were selected based on the matching criteria from index case sites first and then only from sites associated with a trial in Black hypertensives.

The overall sample consisted of 215 subjects including 56 cases with at least one matched control for a total of 159 controls. Race was self-reported as part of each subject's participation in the original omapatrilat phase 11/111 program. The overall sample included a mixture of races, including Blacks, Caucasians and Brazilian subjects. The Brazilian subjects self-reported Mulatto for race. These subjects are referred to as “Other” for race in this study. The statistical analyses described below were performed on the overall sample and four subgroups, including Blacks, Caucasians, Angioedema and Angioedema-like. The Blacks subgroup included 21 angioedema and angioedema-like cases and 51 matched controls. The Caucasians subgroup included 34 angioedema and angioedema-like cases and 107 matched controls. The angioedema subgroup included a mixture of races for a total of 23 cases and 70 matched controls. The angioedema-like subgroup also included a mixture of races for a total of 33 cases and 89 matched controls.

Measures. Single nucleotide polymorphisms (SNPs) in angioedema-susceptibility candidate gene regions (Table I) may be genotyped on all subjects essentially as described in Example 3 herein . The SNPs that are genotyped likely represent a sample of the polymorphic variation in each gene and are not exhaustive with regard to coverage of the total genetic variation that may be present in each gene. Specifically, only those SNPs referenced herein are genotyped and statistically analyzed, as described. The SNPs for which a statistical association to angioedema susceptibility was confirmed are provided in Table XXXXX and described as angioedema-susceptibility candidate genes. Although an association to angioedema susceptibility may not have been observed for the other candidate angioedema SNPs provided in Table I, larger sample populations and/or other genotyping methods may result in an observed association. Thus, all of the SNPs of the present invention should be considered as angioedema-susceptibility candidate genes for the purposes of the present invention.

Statistical Analyses. Conditional logistic regression (HOSMER and LEMESHOW 2000) is used to examine the associations between genotypes of candidate angioedema susceptibility gene SNPs and the development of angioedema or angioedema-like events. All SNPs are bi-allelic with three possible genotypes. For each SNP, in the overall sample and each subgroup, allele frequencies are estimated. For consistency in SNP genotype parameter coding in the logistic regression models, the less frequent allele of each SNP is designated as the rare allele and the number of copies of that allele that each subject carried, either 0, 1, or 2, is then determined. Three possible genotypes for each SNP leaves two degrees of freedom for parameters in the conditional logistic regression model representing the information contained in these three genotype categories. Two dummy variables are therefore created based on the copies of the rare allele for each subject for use in the conditional logistic regression model,

x₁=1 if copies of rare allele=1, 0 otherwise and

x₂=1 if copies of rare allele=2, 0 otherwise.

The full conditional logistic regression model used was

$${π}_{k} ( x ) = \frac{{ⅇ}^{{α}_{k} + {β}_{1}^{′} {x}_{1} + {β}_{2}^{′} {x}_{2}}}{1 + {ⅇ}^{{α}_{k} + {β}_{1}^{′} {x}_{1} + {β}_{2}^{′} {x}_{2}}} ,$$

where x in πₖ (x) is the vector of dummy variables representing the SNP genotypes described above, k is the matching stratum index specific to each matched case-control set of subjects, πₖ (x) is the matching stratum-specific expected probability that a subject is a case given x, αₖ is the matching stratum-specific contribution to πₖ (x) of all the matching variables constant within the kth stratum and each β′ represents the contribution of the respective dummy variable to πₖ (x).

For each SNP, the null hypothesis was that the vector of β′ are all equal to 0 and was tested using the scores test (HOSMER and LEMESHOW 2000). The degrees of freedom for the scores test statistic was equal to one less than the number of genotypes. Exponentiation of each slope coefficient, β′, provided an estimate of the ratio of the odds of an adverse event (angioedema and/or Angioedema-like) in subjects carrying the specified copies of the rare allele represented in the definition of the coefficient, relative to controls matched for nationality, race, gender and starting dose, over the odds of such an adverse event for similarly matched subjects not carrying any copies of the rare allele. 95% confidence interval limits are estimated for each odds ratio based on the standard error estimate of the respective slope coefficient.

The sample sizes for the subgroup analyses (angioedema, angioedema-like, Blacks, Caucasians) are small and unbalanced with regard to the distribution of individuals among SNP genotype classes. Unbalanced genotype numbers are expected in samples from human populations and are also observed for the overall sample. Furthermore, some SNP allele frequencies are very rare. In situations where many or all of these conditions existed, the asymptotic maximum likelihood methods used for parameter estimation with conventional conditional logistic regression may not be reliable and, for SNPs with extreme genotype distributions resulting in zero cells, it is impossible to obtain parameter estimates using these methods. Exact conditional logistic regression is used to supplement the asymptotic methods described above to deal with these estimation problems whenever computationally necessary and feasible (MEHTA and PATEL 1995). LogXact-4® for Windows software was used for all the asymptotic and exact conditional logistic regression parameter estimates (Mehta and Patel 2000).

Since the SNP coverage within each gene was not exhaustive of the genetic variation that may be present and possibly related to event susceptibility in each gene, inferences about these SNP associations with angioedema and/or angioedema-like events for each gene, are therefore related to the hypothesis that genetic variation in that gene may be involved in susceptibility to such events.

The utility, in general, of each of these significant SNP-angioedema and/or angioedema-like event associations is that they suggest (1) such SNPs may be causally involved, alone or in combination with other SNPs in the respective gene regions with susceptibility to angioedema and/or angioedema-like events resulting from exposure to a neutral endopeptidase (NEP) inhibitor and/or an angiotensin converting enzyme (ACE) inhibitor; (2) such SNPs, if not directly causally involved, are reflective of an association because of linkage disequilibrium with one or more other SNPs that may be causally involved, alone or in combination with other SNPs in the respective gene regions with susceptibility to angioedema and/or angioedema-like events resulting from exposure to a neutral endopeptidase (NEP) inhibitor and/or an angiotensin converting enzyme (ACE) inhibitor; (3) such SNPs may be useful in establishing haplotypes that may be used to narrow the search for and identify polymorphisms or combinations of polymorphisms that may be causally, alone or in combination with other SNPs in the respective gene regions with susceptibility to angioedema and/or angioedema-like events resulting from exposure to a neutral endopeptidase (NEP) inhibitor and/or an angiotensin converting enzyme (ACE) inhibitor; and (4) such SNPs, if used to establish haplotypes that are identified as causally involved in such event susceptibility, may be used to predict which subjects are most likely to experience such events when exposed to a neutral endopeptidase (NEP) inhibitor and/or an angiotensin converting enzyme (ACE) inhibitor. The term “respective gene regions” shall be construed to refer to those regions of each gene which have been used to identify the SNPs of the present invention.

Although the association to the angioedema phenotype may be demonstrated herein for less than all of the SNPs of the present invention, at least one or more SNPs may show an association to the angioedema phenotype using the methods essentially as described herein. Such associations are encompassed by the present invention. Moreover, the use of such SNPs for which an association to the angioedema phenotype has either been established or not established are encompassed by the present invention for use in establishing haplotypes to predict which subjects are most likely to experience such events when exposed to a neutral endopeptidase (NEP) inhibitor and/or an angiotensin converting enzyme (ACE) inhibitor.

### References

HOSMER, D. W., and S. LEMESHOW, 2000 Applied logistic regression. John Wiley & Sons, New York.

MEHTA, C., and N. PATEL, 2000 LogXact-4® for Windows, pp. Cytel Software Corporation, Cambridge.

MEHTA, C. R., and N. R. PATEL, 1995 Exact logistic regression: theory and examples. Stat Med 14: 2143-60.

### Example 6

Method of Isolating the Native Forms of the Andioedema Candidate Genes

A number of methods have been described in the art that may be utilized in isolating the native forms of the angioedema candidate genes. Specific methods for each gene are referenced below and which are hereby incorporated by reference herein in their entireties. The artisan, skilled in the molecular biology arts, would be able to isolate these native forms based upon the methods and information contained, and/or referenced, therein.

### Aminopeptidase P (HGNC_ID: XPNPEP2)

1) Venema, R. C., et al., Biochim Biophys Acta Oct. 9, 1997;1354 (1):45-8. Cloning and tissue distribution of human membrane-bound aminopeptidase P.

2) Cottrell, G. S., et al., Biochem Soc Trans August 1998;26 (3):S248. The cloning and functional expression of human pancreatic aminopeptidase P.

3) Sprinkle, T. J., et al., Genomics May 15, 1998;50 (1):114-6. Assignment of the membrane-bound human aminopeptidase P gene (XPNPEP2) to chromosome Xq25.

### Kallikrein 1 (HGNC_ID: KLK1)

1) Fukushima, D., et al., Biochemistry Dec. 31, 1985;24 (27):8037-43. Nucleotide sequence of cloned cDNA for human pancreatic kallikrein.

2) Evans, B. A., et al., Biochemistry May 3, 1988;27 (9):3124-9. Structure and chromosomal localization of the human renal kallikrein gene.

3) Angermann, A., et al., Biochem J Sep. 15, 1989;262 (3):787-93. Cloning and expression of human salivary-gland kallikrein in Escherichia coli.

Methods of isolation for the other angioedema candidate genes of the present invention may be found in reference to the references cited in the Genbank accession nos. for each gene provided herein which are hereby incorporated by reference herein.

### Example 7

Method of Isolating the Polymorphic Forms of the Andioedema Candidate Genes of the Present Invention

Since the allelic genes of the present invention represent genes present within at least a subset of the human population, these genes may be isolated using the methods provided in Example 3 above. For example, the source DNA used to isolate the allelic gene may be obtained through a random sampling of the human population and repeated until the allelic form of the gene is obtained. Preferably, random samples of source DNA from the human population are screened using the SNPs and methods of the present invention to identify those sources that comprise the allelic form of the gene. Once identified, such a source may be used to isolate the allelic form of the gene(s). The invention encompasses the isolation of such allelic genes from both genomic and/or cDNA libraries created from such source(s).

In reference to the specific methods provided in Example 3 above, it is expected that isolating the angioedema candidate genes would be within the skill of an artisan trained in the molecular biology arts. Nonetheless, a detailed exemplary method of isolating at least one of the bradykinin associated genes, in this case the variant form (R119H) of the C1S cDNA (SNP_ID=AE111s17) is provided. Briefly, First, the individuals with the R119H variation are identified by genotyping the genomic DNA samples using the FP-SBE (Chen X 1999) method, described in Examples 1 and 2 above. DNA samples publicly available (e.g., from the Coriell Institute (Collingswood, N.J.) or from the Bristol-Myers Squibb omipatriot clinical samples described herein may be used. Oligonucleotide primers that are used for this genotyping assay are as follows.

| | | | |
|---|---|---|---|
| CIS.L: | 5′-GTGAGCCTTACCTGTGGCAA-3′ | (PCR forward primer) | (SEQ ID NO:1191) |
| CIS.R: | 5′-AAACTCCAGGTGATCTTTAAGTCAGA-3′ | (PCR reverse primer) | (SEQ ID NO:1202) |
| CIS: | 5′-AGACTTTTCCAATGAAGAGC-3′ | (SBE primer) | (SEQ ID NO:1215) |

By analyzing genomic DNA samples, individuals with the R119H form of the CIS variant may be identified. Next, Lymphoblastoid cell lines from these individuals may be obtained from the Coriell Institute. These cells can be grown in RPMI-1640 medium with L-glutamine plus 10% FCS at 37 degrees. PolyA+RNA are then isolated from these cells using Oligotex Direct Kit (Life Technologies).

First strand cDNA (complementary DNA) is produced using Superscript Preamplification System for First Strand cDNA Synthesis (Life Technologies, Cat No 18089-011) using these polyA+RNA as templates, as specified in the users manual which is hereby incorporated herein by reference in its entirety. Specific cDNA encoding the C1S protein is amplified by polymerase chain reaction (PCR) using a forward primer which hybridizes to the 5′-UTR region, a reverse primer which hybridizes to the 3′-UTR region, and these first strand cDNA as templates (Sambrook, Fritsch et al. 1989). For example, the primers specified in Tables IV and/or V may be used. Alternatively, these primers may be designed using Primer3 program (Rozen S 2000). Restriction enzyme sites (example: SalI for the forward primer, and NotI for reverse primer) are added to the 5′-end of these primer sequences to facilitate cloning into expression vectors after PCR amplification. PCR amplification may be performed essentially as described in the owner's manual of the Expand Long Template PCR System (Roche Molecular Biochemicals) following manufacturer's standard protocol, which is hereby incorporated herein by reference in its entirety.

PCR amplification products are digested with restriction enzymes (such as SalI and NotI, for example) and ligated with expression vector DNA cut with the same set of restriction enzymes. pSPORT (Invitrogen) is one example of such an expression vector. After ligated DNA is introduced into E. coli cells (Sambrook, Fritsch et al. 1989), plasmid DNA is isolated from these bacterial cells. This plasmid DNA is sequenced to confirm the presence an intact (full-length) coding region of the human C1S protein with the R119H variation using methods well known in the art and described elsewhere herein.

The skilled artisan would appreciate that the above method may be applied to isolating the other novel polymorphic bradykinin associated genes of the present invention through the simple substitution of applicable PCR and sequencing primers. Such primers may be selected from any one of the applicable primers provided in Tables IV and/or V, or may be designed using the Primer3 program (Rozen S 2000) as described. Such primers may preferably comprise at least a portion of any one of the polynucleotide sequences of the present invention.

### Example 8

Method of Engineering the Allelic Forms of the Andioedema Candidate Genes of the Present Invention

Aside from isolating the allelic genes of the present invention from DNA samples obtained from the human population and/or the Coriell Institute, as described in Example 4 above, the invention also encompasses methods of engineering the allelic genes of the present invention through the application of site-directed mutagenesis to the isolated native forms of the genes. Such methodology could be applied to synthesize allelic forms of the genes comprising at least one, or more, of the encoding SNPs of the present invention (e.g., silent, missense)—preferably at least 1, 2, 3, or 4 encoding SNPs for each gene.

In reference to the specific methods provided in Example 4 above, it is expected that isolating the novel polymorphic angioedema candidate genes of the present invention would be within the skill of an artisan trained in the molecular biology arts. Nonetheless, a detailed exemplary method of engineering at least one of the bradykinin associated genes to comprise the encoding and/or non-coding polymorphic nucleic acid sequence, in this case the variant form (R119H) of C1S cDNA (SNP_ID=AE111s17) is provided. Briefly, cDNA clones encoding the human C1S protein may be identified by homology searches with the BLASTN program (Altschul SF 1990) against the Genbank non-redundant nucleotide sequence database using the published human C1S cDNA sequence (GenBank Accession No.: NM_{—}001734.1). After obtaining these clones, they are sequenced to confirm the validity of the DNA sequences.

Once these clones are confirmed to contain the intact wild type cDNA sequence of the C1S coding region, the R119H polymorphism (mutation) may be introduced into the native sequence using PCR directed in vitro mutagenesis (Cormack 2000). In this method, synthetic oligonucleotides are designed to incorporate a point mutation at one end of an amplified fragment. Following PCR, the amplified fragments are made blunt-ended by treatment with Klenow Fragment. These fragments are then ligated and subcloned into a vector to facilitate sequIence analysis. This method consists of the following steps.

1. Subcloning of cDNA insert into a high copy plasmid vector containing multiple cloning sites and M13 flanking sequences, such as pUC19 (Sambrook, Fritsch et al. 1989), in the forward orientation. The skilled artisan would appreciate that other plasmids could be equally substituted, and may be desirable in certain circumstances.

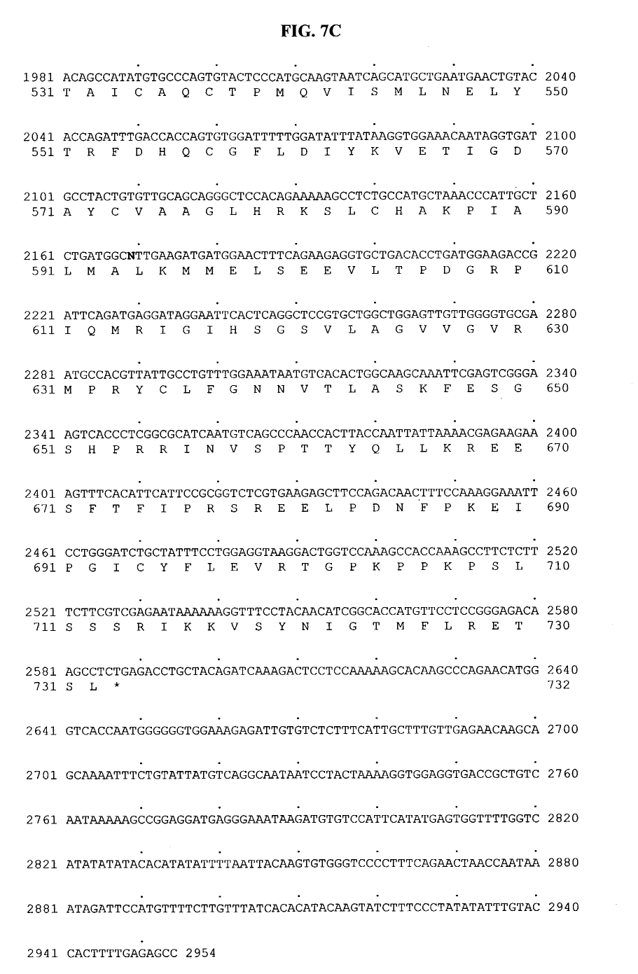

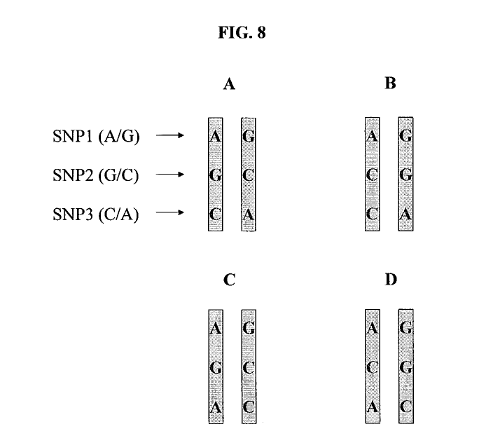

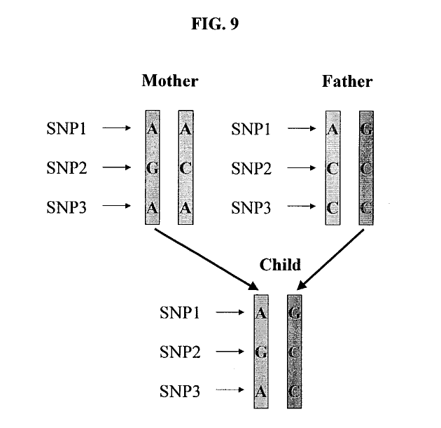

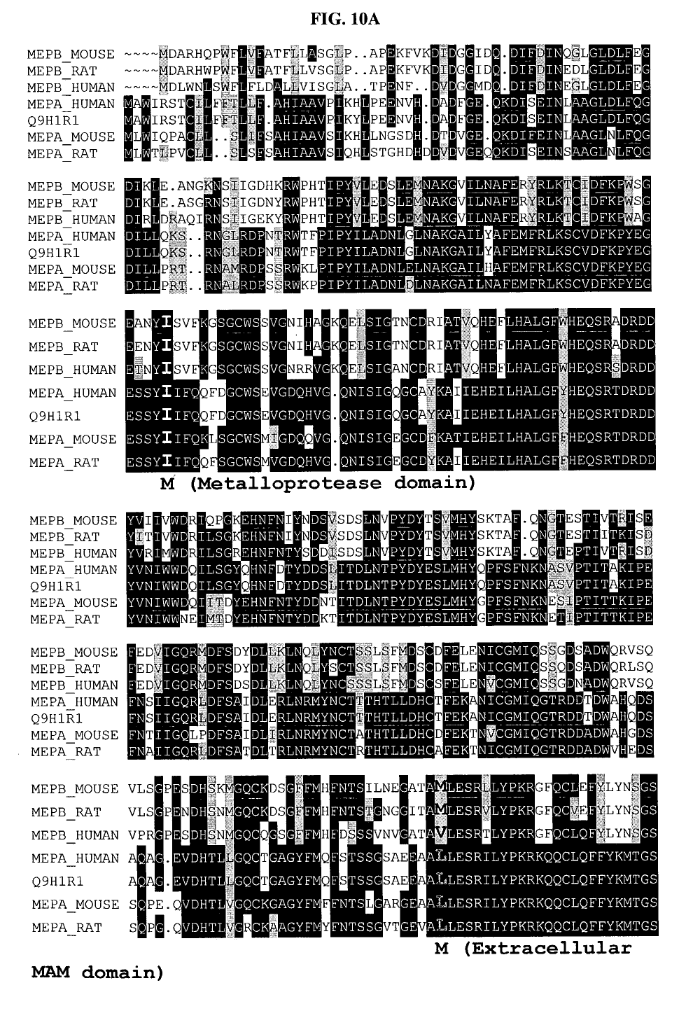

2. Introduction of a mutation by PCR amplification of the cDNA region downstream of the mutation site using a primer including the mutation. (FIG. 8.5.2 in (Cormack 2000)). In the case of introducing the R119H mutation into the human C1S protein, the following two primers may be used.

M13 reverse sequencing primer

5′-AGCGGATAACAATTTCACACAGGA-3′ (SEQ ID NO:1216).

Mutation primer

5′-ATTTTACGGGGTTTGCTGCAT-3′ (SEQ ID NO:1217).

Mutation primer contains the mutation (R119H) at the 5′ end and its downstream flanking sequence. M13 reverse sequencing primer hybridizes to the pUC19 vector. Subcloned cDNA comprising the human C1S protein is used as a template (described in Step 1). A 100 ul PCR reaction mixture is prepared using long of the template DNA, 200 uM 4 dNTPs, 1 uM primers, 0.25 U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5 U Klenow Fragment is added and incubated for 15 min at 30 degrees. The PCR product is then digested with the restriction enzyme, EcoRI.

3. PCR amplification of the upstream region is then performed, using subcloned cDNA as a template (the product of Step 1). This PCR is done using the following two primers:

M13 forward sequencing primer 5′-CGCCAGGGTTTTCCCAGTCACGAC-3′ (SEQ ID NO:1218).

Flanking primer 5′-GCTCTTCATTGGAAAAGTCT-3′ (SEQ ID NO:1219).

Flanking primer is complimentary to the upstream flanking sequence of the R119H mutation. M13 forward sequencing primer hybridizes to the pUC19 vector. PCR conditions and Klenow treatments follow the same procedures as provided in Step 2, above. The PCR product is then digested with the restriction enzyme, HindIII.

4. Prepare the pUC19 vector for cloning the cDNA comprising the polymorphic site. Digest pUC19 plasmid DNA with EcoRI and HindII. The resulting digested vector fragment may then be purified using techniques well known in the art, such as gel purification, for example.

5. Combine the products from Step 2 (PCR product containing mutation), Step 3 (PCR product containing the upstream region), and Step 4 (digested vector), and ligate them together using standard blunt-end ligation conditions (Sambrook, Fritsch et al. 1989).

6. Transform the resulting recombinant plasmid from Step 5 into E. coli competent cells using methods known in the art, such as, for example, the transformation methods described in Sambrook, Fritsch et al. 1989.

7. Analyze the amplified fragment portion of the plasmid DNA by DNA sequencing to confirm the point mutation, and absence of any other mutations introduced during PCR. The method of sequencing the insert DNA, including the primers utilized, are described herein or are otherwise known in the art.

The skilled artisan would appreciate that the above method may be applied to engineering the other polymorphic bradykinin associated genes of the present invention through the simple subsitution of applicable mutation, flanking, PCR, and sequencing primers for each specific gene and/or polymorphism. Some of these primers may be selected from any one of the applicable primers provided in the Tables herein, or may be designed using the Primer3 program (Rozen S 2000), or designed manually, as described. Such primers may preferably comprise at least a portion of any one of the polynucleotide sequences of the present invention.

Moreover, the skilled artisan would appreciate that the above method may be applied to engineering more than one polymorphic nucleic acid sequence of the present invention into the novel polymorphic angioedema associated genes of the present invention. Such an engineered gene could be created through succesive rounds of site-directed mutagenesis, as described in Steps 1 thru 7 above, or consolidated into a single round of mutagenesis. For example, Step 2 above could be performed for each mutation, then the products of both mutation amplifications could be combined with the product of Step 3 and 4, and the procedure followed as described.

### Example 9

Alternative Methods of Detecting Polymorphisms Encompassed by the Present Invention

### Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed. For example, if the target nucleic acid is a cytochrome P450, the liver is a suitable source.

Many of the methods described below require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86, 1173 (1989), and self-sustained sequence replication (Guatelli et al., Proc. Nat. Acad. Sci. USA, 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Additional methods of amplification are known in the art or are described elsewhere herein.

### Detection of Polymorphisms in Target DNA

There are two distinct types of analysis of target DNA for detecting polymorphisms. The first type of analysis, sometimes referred to as de novo characterization, is carried out to identify polymorphic sites not previously characterized (i.e., to identify new polymorphisms). This analysis compares target sequences in different individuals to identify points of variation, i.e., polymorphic sites. By analyzing groups of individuals representing the greatest ethnic diversity among humans and greatest breed and species variety in plants and animals, patterns characteristic of the most common alleles/haplotypes of the locus can be identified, and the frequencies of such alleles/haplotypes in the population can be determined. Additional allelic frequencies can be determined for subpopulations characterized by criteria such as geography, race, or gender. The de novo identification of polymorphisms of the invention is described in the Examples section.

The second type of analysis determines which form(s) oaf characterized (known) polymorphism are present in individuals under test. Additional methods of analysis are known in the art or are described elsewhere herein.

### Allele-Specific Probes

The design and use of allele-specific probes for analyzing polymorphisms is described by e.g., Saiki et al., Nature 324,163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

### Tiling Arrays

The polymorphisms can also be identified by hybridization to nucleic acid arrays, some examples of which are described in WO 95/11995. The same arrays or different arrays can be used for analysis of characterized polymorphisms. -WO 95/11995 also describes sub arrays that are optimized for detection of a variant form of a precharacterized polymorphism. Such a sub array contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to bases).

### Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17,2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing elongation from the primer (see, e.g., WO 93/22456).

### Direct-Sequencing

The direct analysis of the sequence of polymorphisms of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

### Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology. Principles and Applications for DNA Amplification, (W H. Freeman and Co, New York, 1992), Chapter 7.

### Single-Strand Conformation Polymorphism Analysis

Alleles of target sequences can be differentiated using single-strand Conformation polymorphism analysis, which identifies base differences by alteration in electrophoretic migration of single stranded PCR products, as described in Orita et al., Proc. Nat. Acad. Sci. 86,2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

### Single Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997), uses a locus-specific oligonucleotide primer labeled on the 5′ terminus with 5-carboxyfluorescein (F AM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs) in dye-terminator sequencing fashion. An increase in fluorescence of the added ddNTP in response to excitation at the wavelength of the labeled primer is used to infer the identity of the added nucleotide.

### Example 10

Bacterial Expression of a Polypeptide

A polynucleotide encoding a polypeptide of the present invention is amplified using PCR oligonucleotide primers corresponding to the 5′ and 3′ ends of the DNA sequence, as outlined in the Examples above or otherwise known in the art, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM imidazole. Imidazole is removed by a final dialyzing step against PBS or 50 mM sodium acetate pH 6 buffer plus 200 mM NaCl. The purified protein is stored at 4 degree C. or frozen at −80 degree C.

### Example 11

Purification of a Polypeptide from an Inclusion Body

The following alternative method can be used to purify a polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfluidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perceptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perceptive Biosystems) and weak anion (Poros CM-20, Perceptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A280 monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Coomassie blue stained 16% SDS-PAGE gel when 5 ug of purified protein is loaded. The purified protein can also be tested for endotoxin/LPS contamination, and typically the LPS content is less than 0.1 ng/ml according to LAL assays.

### Example 12

Cloning and Expression of a Polypeptide in a Baculovirus Expression System

In this example, the plasmid shuttle vector pAc373 is used to insert a polynucleotide into a baculovirus to express a polypeptide. A typical baculovirus expression vector contains the strong polyhedrin promoter of the Autographa californica nuclear polyhedrosis virus (AcMNPV) followed by convenient restriction sites, which may include, for example BamHI, XbaI and Asp718. The polyadenylation site of the simian virus 40 (“SV40”) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

A polynucleotide encoding a polypeptide of the present invention is amplified using PCR oligonucleotide primers corresponding to the 5′ and 3′ ends of the DNA sequence, as outlined in the Examples above or otherwise known in the art, to synthesize insertion fragments. The primers used to amplify the cDNA insert should preferably contain restriction sites at the 5′ end of the primers in order to clone the amplified product into the expression vector. Specifically, the cDNA sequence contained in the deposited clone, including the AUG initiation codon and the naturally associated leader sequence identified elsewhere herein (if applicable), is amplified using the PCR protocol described herein. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures” Texas Agricultural Experimental Station Bulletin No. 1555 (1987).

The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transformed with 1.0 ug of a commercially available linearized baculovirus DNA (“BaculoGoldtm baculovirus DNA”, Pharmingen, San Diego, Calif.), using the lipofection method described by Felgner et al., Proc. Natl. Acad. Sci. USA 84:7413-7417 (1987). One ug of BaculoGoldtm virus DNA and Sug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of 35S-methionine and 5 uCi 35S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 13

Expression of a Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem . . . 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transformation. Five μg of an expression plasmid is cotransformed with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of the desired gene product is analyzed, for instance, by SDS-PAGE and Western blot or by reversed phase HPLC analysis.

### Example 14

Production of an Antibody from a Polypeptide

The antibodies of the present invention can be prepared by a variety of methods. (See, Current Protocols, Chapter 2.) As one example of such methods, cells expressing a polypeptide of the present invention are administered to an animal to induce the production of sera containing polyclonal antibodies. In a preferred method, a preparation of the protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Kohler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C.), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F (ab′)2 and other fragments of the antibodies of the present invention may be used according to the methods disclosed herein. Such fragments are typically produced by proteolytic cleavage, using enzymes such as papain (to produce Fab fragments) or pepsin (to produce F (ab′)2 fragments). Alternatively, protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

### Example 15

Method of Detecting Abnormal Levels of a Polypeptide in a Biological Sample

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

For example, antibody-sandwich ELISAs are used to detect polypeptides in a sample, preferably a biological sample. Wells of a microtiter plate are coated with specific antibodies, at a final concentration of 0.2 to 10 ug/ml. The antibodies are either monoclonal or polyclonal and are produced by the method described elsewhere herein. The wells are blocked so that non-specific binding of the polypeptide to the well is reduced.

The coated wells are then incubated for >2 hours at RT with a sample containing the polypeptide. Preferably, serial dilutions of the sample should be used to validate results. The plates are then washed three times with deionized or distilled water to remove unbounded polypeptide.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 16

Method of Treatment Using Gene Therapy—Ex vivo

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 degree C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in the Examples herein or otherwise known in the art, using primers and having appropriate restriction sites and initiation/stop codons, if necessary. Preferably, the 5′ primer contains an EcoRI site and the 3′ primer includes a HindIIIl site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector has the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

### Example 17

Method of Treatment Using Gene Therapy—In vivo

Another aspect of the present invention is using in vivo gene therapy methods to treat disorders, diseases and conditions. The gene therapy method relates to the introduction of naked nucleic acid (DNA, RNA, and antisense DNA or RNA) sequences into an animal to increase or decrease the expression of the polypeptide. The polynucleotide of the present invention may be operatively linked to a promoter or any other genetic elements necessary for the expression of the polypeptide by the target tissue. Such gene therapy and delivery techniques and methods are known in the art, see, for example, WO90/11092, WO98/11779; U.S. Pat. Nos. 5,693,622, 5,705,151, 5,580,859; Tabataet al., Cardiovasc. Res. 35 (3):470-479 (1997); Chao et al., Pharmacol. Res. 35 (6):517-522 (1997); Wolff, Neuromuscul. Disord. 7 (5):314-318 (1997); Schwartz et al., Gene Ther. 3 (5):405-411 (1996); Tsurumi et al., Circulation 94 (12):3281-3290 (1996) (incorporated herein by reference).

The polynucleotide constructs may be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Felgner P. L. et al. (1995) Ann. NY Acad. Sci. 772:126-139 and Abdallah B. et al. (1995) Biol. Cell 85 (1):1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for protein expression. A time course for protein expression may be done in a similar fashion except that quadriceps from different mice are harvested at different times. Persistence of DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using naked DNA.

### Example 20

The Effect of the Polypeptides of the Invention on the Growth of Vascular Endothelial Cells

On day 1, human umbilical vein endothelial cells (HUVEC) are seeded at 2-5×104 cells/35 mm dish density in M199 medium containing 4% fetal bovine serum (FBS), 16 units/ml heparin, and 50 units/ml endothelial cell growth supplements (ECGS, Biotechnique, Inc.). On day 2, the medium is replaced with M199 containing 10% FBS, 8 units/ml heparin. A polypeptide having the amino acid sequence described herein, and positive controls, such as VEGF and basic FGF (bFGF) are added, at varying concentrations. On days 4 and 6, the medium is replaced. On day 8, cell number is determined with a Coulter Counter.

An increase in the number of HUVEC cells indicates that the polypeptide of the invention may proliferate vascular endothelial cells.

One skilled in the art could easily modify the exemplified studies to test the activity of polynucleotides of the invention (e.g., gene therapy), agonists, and/or antagonists of polynucleotides or polypeptides of the invention.

### Example 21

Stimulatory Effect of Polypeptides of the Invention of the Proliferation of Vascular Endothelial Cells

For evaluation of mitogenic activity of growth factors, the colorimetric MTS (3-(4,5-dimethylthiazol-2-yl)-5-(3-carboxymethoxyphenyl)-2-(4-sulfophenyl)2H-tetrazolium) assay with the electron coupling reagent PMS (phenazine methosulfate) was performed (CellTiter 96 AQ, Promega). Cells are seeded in a 96-well plate (5,000 cells/well) in 0.1 mL serum-supplemented medium and are allowed to attach overnight. After serum-starvation for 12 hours in 0.5% FBS, conditions (bFGF, VEGF165 or a polypeptide of the invention in 0.5% FBS) with or without Heparin (8 U/ml) are added to wells for 48 hours. 20 mg of MTS/PMS mixture (1:0.05) are added per well and allowed to incubate for 1 hour at 37° C. before measuring the absorbance at 490 nm in an ELISA plate reader. Background absorbance from control wells (some media, no cells) is subtracted, and seven wells are performed in parallel for each condition. See, Leak et al. In Vitro Cell. Dev. Biol. 30A:512-518 (1994).

One skilled in the art could easily modify the exemplified studies to test the activity of polynucleotides of the invention (e.g., gene therapy), agonists, and/or antagonists of polynucleotides or polypeptides of the invention.

### Example 22

Inhibition of PDGF-Induced Vascular Smooth Muscle Cell Proliferation Stimulatory Effect

HAoSMC proliferation can be measured, for example, by BrdUrd incorporation. Briefly, subconfluent, quiescent cells grown on the 4-chamber slides are transfected with CRP or FITC-labeled AT2-3LP. Then, the cells are pulsed with 10% calf serum and 6 mg/ml BrdUrd. After 24 h, immunocytochemistry is performed by using BrdUrd Staining Kit (Zymed Laboratories). In brief, the cells are incubated with the biotinylated mouse anti-BrdUrd antibody at 4 degrees C. for 2 h after being exposed to denaturing solution and then incubated with the streptavidin-peroxidase and diaminobenzidine. After counterstaining with hematoxylin, the cells are mounted for microscopic examination, and the BrdUrd-positive cells are counted. The BrdUrd index is calculated as a percent of the BrdUrd-positive cells to the total cell number. In addition, the simultaneous detection of the BrdUrd staining (nucleus) and the FITC uptake (cytoplasm) is performed for individual cells by the concomitant use of bright field illumination and dark field-UV fluorescent illumination. See, Hayashida et al., J. Biol. Chem. 6:271 (36):21985-21992 (1996).

One skilled in the art could easily modify the exemplified studies to test the activity of polynucleotides of the invention (e.g., gene therapy), agonists, and/or antagonists of polynucleotides or polypeptides of the invention.

### Example 23

Stimulation of Endothelial Migration

This example will be used to explore the possibility that a polypeptide of the invention may stimulate lymphatic endothelial cell migration.

Endothelial cell migration assays are performed using a 48 well microchemotaxis chamber (Neuroprobe Inc., Cabin John, MD; Falk, W., et al., J. Immunological Methods 1980;33:239-247). Polyvinylpyrrolidone-free polycarbonate filters with a pore size of 8 um (Nucleopore Corp. Cambridge, Mass.) are coated with 0.1% gelatin for at least 6 hours at room temperature and dried under sterile air. Test substances are diluted to appropriate concentrations in M199 supplemented with 0.25% bovine serum albumin (BSA), and 25 ul of the final dilution is placed in the lower chamber of the modified Boyden apparatus. Subconfluent, early passage (2-6) HUVEC or BMEC cultures are washed and trypsinized for the minimum time required to achieve cell detachment. After placing the filter between lower and upper chamber, 2.5×105 cells suspended in 50 ul M199 containing 1% FBS are seeded in the upper compartment. The apparatus is then incubated for 5 hours at 37° C. in a humidified chamber with 5% CO2 to allow cell migration. After the incubation period, the filter is removed and the upper side of the filter with the non-migrated cells is scraped with a rubber policeman. The filters are fixed with methanol and stained with a Giemsa solution (Diff-Quick, Baxter, McGraw Park, Ill.). Migration is quantified by counting cells of three random high-power fields (40×) in each well, and all groups are performed in quadruplicate.

One skilled in the art could easily modify the exemplified studies to test the activity of polynucleotides of the invention (e.g., gene therapy), agonists, and/or antagonists of polynucleotides or polypeptides of the invention.

### Example 24

Stimulation of Nitric Oxide Production by Endothelial Cells

Nitric oxide released by the vascular endothelium is believed to be a mediator of vascular endothelium relaxation. Thus, activity of a polypeptide of the invention can be assayed by determining nitric oxide production by endothelial cells in response to the polypeptide.

Nitric oxide is measured in 96-well plates of confluent microvascular endothelial cells after 24 hours starvation and a subsequent 4 hr exposure to various levels of a positive control (such as VEGF-1) and the polypeptide of the invention. Nitric oxide in the medium is determined by use of the Griess reagent to measure total nitrite after reduction of nitric oxide-derived nitrate by nitrate reductase. The effect of the polypeptide of the invention on nitric oxide release is examined on HUVEC.

Briefly, NO release from cultured HUVEC monolayer is measured with a NO-specific polarographic electrode connected to a NO meter (Iso-NO, World Precision Instruments Inc.) (1049). Calibration of the NO elements is performed according to the following equation:

2 KNO2+2KI+2H2SO4 6 2NO+I2+2H2O+2K2SO4

The standard calibration curve is obtained by adding graded concentrations of KNO2 (0, 5, 10, 25, 50, 100, 250, and 500 nmol/L) into the calibration solution containing KI and H2SO4. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) To maintain the temperature at 37° C. The NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×106 endothelial cells. All values reported are means of four to six measurements in each group (number of cell culture wells). See, Leak et al. Biochem. and Biophys. Res. Comm. 217:96-105 (1995).

One skilled in the art could easily modify the exemplified studies to test the activity of polynucleotides of the invention (e.g., gene therapy), agonists, and/or antagonists of polynucleotides or polypeptides of the invention.

### Example 25

Effect of Polypepides of the Invention on Cord Formation in Angiogenesis

Another step in angiogenesis is cord formation, marked by differentiation of endothelial cells. This bioassay measures the ability of microvascular endothelial cells to form capillary-like structures (hollow structures) when cultured in vitro.

CADMEC (microvascular endothelial cells) are purchased from Cell Applications, Inc. as proliferating (passage 2) cells and are cultured in Cell Applications' CADMEC Growth Medium and used at passage 5. For the in vitro angiogenesis assay, the wells of a 48-well cell culture plate are coated with Cell Applications' Attachment Factor Medium (200 ml/well) for 30 min. at 37° C. CADMEC are seeded onto the coated wells at 7,500 cells/well and cultured overnight in Growth Medium. The Growth Medium is then replaced with 300 mg Cell Applications' Chord Formation Medium containing control buffer or a polypeptide of the invention (0.1 to 100 ng/ml) and the cells are cultured for an additional 48 hr. The numbers and lengths of the capillary-like chords are quantitated through use of the Boeckeler VIA-170 video image analyzer. All assays are done in triplicate.

Commercial (R&D) VEGF (50 ng/ml) is used as a positive control. b-esteradiol (1 ng/ml) is used as a negative control. The appropriate buffer (without protein) is also utilized as a control.

One skilled in the art could easily modify the exemplified studies to test the activity of polynucleotides of the invention (e.g., gene therapy), agonists, and/or antagonists of polynucleotides or polypeptides of the invention.

### Example 26

Angiogenic Effect on Chick Chorioallantoic Membrane

Chick chorioallantoic membrane (CAM) is a well-established system to examine angiogenesis. Blood vessel formation on CAM is easily visible and quantifiable. The ability of polypeptides of the invention to stimulate angiogenesis in CAM can be examined.

Fertilized eggs of the White Leghorn chick (Gallus gallus) and the Japanese qual (Coturnix coturnix) are incubated at 37.8° C. and 80% humidity. Differentiated CAM of 16-day-old chick and 13-day-old qual embryos is studied with the following methods.

On Day 4 of development, a window is made into the egg shell of chick eggs. The embryos are checked for normal development and the eggs sealed with cellotape. They are further incubated until Day 13. Thermanox coverslips (Nunc, Naperville, Ill.) are cut into disks of about 5 mm in diameter. Sterile and salt-free growth factors are dissolved in distilled water and about 3.3 mg/5 ml are pipetted on the disks. After air-drying, the inverted disks are applied on CAM. After 3 days, the specimens are fixed in 3% glutaraldehyde and 2% formaldehyde and rinsed in 0.12 M sodium cacodylate buffer. They are photographed with a stereo microscope [Wild M8] and embedded for semi- and ultrathin sectioning as described above. Controls are performed with carrier disks alone.

One skilled in the art could easily modify the exemplified studies to test the activity of polynucleotides of the invention (e.g., gene therapy), agonists, and/or antagonists of polynucleotides or polypeptides of the invention.

### Example 27

Angiogenesis Assay Using a Matrigel Implant in Mouse

In vivo angiogenesis assay of a polypeptide of the invention measures the ability of an existing capillary network to form new vessels in an implanted capsule of murine extracellular matrix material (Matrigel). The protein is mixed with the liquid Matrigel at 4 degree C. and the mixture is then injected subcutaneously in mice where it solidifies. After 7 days, the solid “plug” of Matrigel is removed and examined for the presence of new blood vessels. Matrigel is purchased from Becton Dickinson Labware/Collaborative Biomedical Products.

When thawed at 4 degree C. the Matrigel material is a liquid. The Matrigel is mixed with a polypeptide of the invention at 150 ng/ml at 4 degrees C. and drawn into cold 3 ml syringes. Female C57B1/6 mice approximately 8 weeks old are injected with the mixture of Matrigel and experimental protein at 2 sites at the midventral aspect of the abdomen (0.5 ml/site). After 7 days, the mice are sacrificed by cervical dislocation, the Matrigel plugs are removed and cleaned (i.e., all clinging membranes and fibrous tissue is removed). Replicate whole plugs are fixed in neutral buffered 10% formaldehyde, embedded in paraffin and used to produce sections for histological examination after staining with Masson's Trichrome. Cross sections from 3 different regions of each plug are processed. Selected sections are stained for the presence of vWF. The positive control for this assay is bovine basic FGF (150 ng/ml). Matrigel alone is used to determine basal levels of angiogenesis.

One skilled in the art could easily modify the exemplified studies to test the activity of polynucleotides of the invention (e.g., gene therapy), agonists, and/or antagonists of polynucleotides or polypeptides of the invention.

### Example 28

Rescue of Ischemia in Rabbit Lower Limb Model

To study the in vivo effects of polynucleotides and polypeptides of the invention on ischemia, a rabbit hindlimb ischemia model is created by surgical removal of one femoral arteries as described previously (Takeshita et al., Am J. Pathol 147:1649-1660 (1995)). The excision of the femoral artery results in retrograde propagation of thrombus and occlusion of the external iliac artery. Consequently, blood flow to the ischemic limb is dependent upon collateral vessels originating from the internal iliac artery (Takeshitaet al. Am J. Pathol 147:1649-1660 (1995)). An interval of 10 days is allowed for post-operative recovery of rabbits and development of endogenous collateral vessels. At 10 day post-operatively (day 0), after performing a baseline angiogram, the internal iliac artery of the ischemic limb is transfected with 500 mg naked expression plasmid containing a polynucleotide of the invention by arterial gene transfer technology using a hydrogel-coated balloon catheter as described (Riessen et al. Hum Gene Ther. 4:749-758 (1993); Leclerc et al. J. Clin. Invest. 90: 936-944 (1992)). When a polypeptide of the invention is used in the treatment, a single bolus of 500 mg polypeptide of the invention or control is delivered into the internal iliac artery of the ischemic limb over a period of 1 min. through an infusion catheter. On day 30, various parameters are measured in these rabbits: (a) BP ratio—The blood pressure ratio of systolic pressure of the ischemic limb to that of normal limb; (b) Blood Flow and Flow Reserve—Resting FL: the blood flow during undilated condition and Max FL: the blood flow during fully dilated condition (also an indirect measure of the blood vessel amount) and Flow Reserve is reflected by the ratio of max FL: resting FL; (c) Angiographic Score—This is measured by the angiogram of collateral vessels. A score is determined by the percentage of circles in an overlaying grid that with crossing opacified arteries divided by the total number m the rabbit thigh; (d) Capillary density—The number of collateral capillaries determined in light microscopic sections taken from hindlimbs.

One skilled in the art could easily modify the exemplified studies to test the activity of polynucleotides of the invention (e.g., gene therapy), agonists, and/or antagonists of polynucleotides or polypeptides of the invention.

### Example 29

Effect of Polypeptides of the Invention on Vasodilation

Since dilation of vascular endothelium is important in reducing blood pressure, the ability of polypeptides of the invention to affect the blood pressure in spontaneously hypertensive rats (SHR) is examined. Increasing doses (0, 10, 30, 100, 300, and 900 mg/kg) of the polypeptides of the invention are administered to 13-14 week old spontaneously hypertensive rats (SHR). Data are expressed as the mean +/− SEM. Statistical analysis are performed with a paired t-test and statistical significance is defined as p<0.05 vs. the response to buffer alone.

One skilled in the art could easily modify the exemplified studies to test the activity of polynucleotides of the invention (e.g., gene therapy), agonists, and/or antagonists of polynucleotides or polypeptides of the invention.

### Example 30

Rat Ischemic Skin Flap Model

The evaluation parameters include skin blood flow, skin temperature, and factor VIII immunohistochemistry or endothelial alkaline phosphatase reaction. Expression of polypeptides of the invention, during the skin ischemia, is studied using in situ hybridization.

The study in this model is divided into three parts as follows:

a) Ischemic skin

b) Ischemic skin wounds

c) Normal wounds

The experimental protocol includes:

a) Raising a 3×4 cm, single pedicle full-thickness random skin flap (myocutaneous flap over the lower back of the animal).

b) An excisional wounding (4-6 mm in diameter) in the ischemic skin (skin-flap).

c) Topical treatment with a polypeptide of the invention of the excisional wounds (day 0, 1, 2, 3, 4 post-wounding) at the following various dosage ranges: 1 mg to 100 mg.

d) Harvesting the wound tissues at day 3, 5, 7, 10, 14 and 21 post-wounding for histological, immunohistochemical, and in situ studies.

One skilled in the art could easily modify the exemplified studies to test the activity of polynucleotides of the invention (e.g., gene therapy), agonists, and/or antagonists of polynucleotides or polypeptides of the invention.

### Example 31

Peripheral Arterial Disease Model

Angiogenic therapy using a polypeptide of the invention is a novel therapeutic strategy to obtain restoration of blood flow around the ischemia in case of peripheral arterial diseases. The experimental protocol includes:

a) One side of the femoral artery is ligated to create ischemic muscle of the hindlimb, the other side of hindlimb serves as a control.

b) A polypeptide of the invention, in a dosage range of 20 mg-500 mg, is delivered intravenously and/or intramuscularly 3 times (perhaps more) per week for 2-3 weeks.

c) The ischemic muscle tissue is collected after ligation of the femoral artery at 1, 2, and 3 weeks for the analysis of expression of a polypeptide of the invention and histology. Biopsy is also performed on the other side of normal muscle of the contralateral hindlimb.

One skilled in the art could easily modify the exemplified studies to test the activity of polynucleotides of the invention (e.g., gene therapy), agonists, and/or antagonists of polynucleotides or polypeptides of the invention.

### Example 32

Ischemic Myocardial Disease Model

A polypeptide of the invention is evaluated as a potent mitogen capable of stimulating the development of collateral vessels, and restructuring new vessels after coronary artery occlusion. Alteration of expression of the polypeptide is investigated in situ. The experimental protocol includes:

a) The heart is exposed through a left-side thoracotomy in the rat. Immediately, the left coronary artery is occluded with a thin suture (6-0) and the thorax is closed.

b) A polypeptide of the invention, in a dosage range of 20 mg-500 mg, is delivered intravenously and/or intramuscularly 3 times (perhaps more) per week for 2-4 weeks.

c) Thirty days after the surgery, the heart is removed and cross-sectioned for morphometric and in situ analyzes.

One skilled in the art could easily modify the exemplified studies to test the activity of polynucleotides of the invention (e.g., gene therapy), agonists, and/or antagonists of polynucleotides or polypeptides of the invention.

### Example 33

Lymphedema Animal Model

The purpose of this experimental approach is to create an appropriate and consistent lymphedema model for testing the therapeutic effects of a polypeptide of the invention in lymphangiogenesis and re-establishment of the lymphatic circulatory system in the rat hind limb. Effectiveness is measured by swelling volume of the affected limb, quantification of the amount of lymphatic vasculature, total blood plasma protein, and histopathology. Acute lymphedema is observed for 7-10 days. Perhaps more importantly, the chronic progress of the edema is followed for up to 3-4 weeks.

Prior to beginning surgery, blood sample is drawn for protein concentration analysis. Male rats weighing approximately ˜350 g are dosed with Pentobarbital. Subsequently, the right legs are shaved from knee to hip. The shaved area is swabbed with gauze soaked in 70% EtOH. Blood is drawn for serum total protein testing. Circumference and volumetric measurements are made prior to injecting dye into paws after marking 2 measurement levels (0.5 cm above heel, at mid-pt of dorsal paw). The intradermal dorsum of both right and left paws are injected with 0.05 ml of 1% Evan's Blue. Circumference and volumetric measurements are then made following injection of dye into paws.

Using the knee joint as a landmark, a mid-leg inguinal incision is made circumferentially allowing the femoral vessels to be located. Forceps and hemostats are used to dissect and separate the skin flaps. After locating the femoral vessels, the lymphatic vessel that runs along side and underneath the vessel(s) is located. The main lymphatic vessels in this area are then electrically coagulated suture ligated.

Using a microscope, muscles in back of the leg (near the semitendinosis and adductors) are bluntly dissected. The popliteal lymph node is then located. The 2 proximal and 2 distal lymphatic vessels and distal blood supply of the popliteal node are then and ligated by suturing. The popliteal lymph node, and any accompanying adipose tissue, is then removed by cutting connective tissues.

Care is taken to control any mild bleeding resulting from this procedure. After lymphatics are occluded, the skin flaps are sealed by using liquid skin (Vetbond) (AJ Buck). The separated skin edges are sealed to the underlying muscle tissue while leaving a gap of ˜0.5 cm around the leg. Skin also may be anchored by suturing to underlying muscle when necessary.

To avoid infection, animals are housed individually with mesh (no bedding). Recovering animals are checked daily through the optimal edematous peak, which typically occurred by day 5-7. The plateau edematous peak are then observed. To evaluate the intensity of the lymphedema, the circumference and volumes of 2 designated places on each paw before operation and daily for 7 days are measured. The effect plasma proteins on lymphedema is determined and whether protein analysis is a useful testing perimeter is also investigated. The weights of both control and edematous limbs are evaluated at 2 places. Analysis is performed in a blind manner.

Circumference Measurements: Under brief gas anesthetic to prevent limb movement, a cloth tape is used to measure limb circumference. Measurements are done at the ankle bone and dorsal paw by 2 different people then those 2 readings are averaged. Readings are taken from both control and edematous limbs.

Volumetric Measurements: On the day of surgery, animals are anesthetized with Pentobarbital and are tested prior to surgery. For daily volumetrics animals are under brief halothane anesthetic (rapid immobilization and quick recovery), both legs are shaved and equally marked using waterproof marker on legs. Legs are first dipped in water, then dipped into instrument to each marked level then measured by Buxco edema software (Chen/Victor). Data is recorded by one person, while the other is dipping the limb to marked area.

Blood-plasma protein measurements: Blood is drawn, spun, and serum separated prior to surgery and then at conclusion for total protein and Ca2+ comparison.

Limb Weight Comparison: After drawing blood, the animal is prepared for tissue collection. The limbs are amputated using a quillitine, then both experimental and control legs are cut at the ligature and weighed. A second weighing is done as the tibio-cacaneal joint is disarticulated and the foot is weighed.

Histological Preparations: The transverse muscle located behind the knee (popliteal) area is dissected and arranged in a metal mold, filled with freezeGel, dipped into cold methylbutane, placed into labeled sample bags at −80 EC until sectioning. Upon sectioning, the muscle is observed under fluorescent microscopy for lymphatics.

One skilled in the art could easily modify the exemplified studies to test the activity of polynucleotides of the invention (e.g., gene therapy), agonists, and/or antagonists of polynucleotides or polypeptides of the invention.

### Example 34

Suppression of TNF Alpha-Induced Adhesion Molecule Expression by a Polypeptide of the Invention

The recruitment of lymphocytes to areas of inflammation and angiogenesis involves specific receptor-ligand interactions between cell surface adhesion molecules (CAMs) on lymphocytes and the vascular endothelium. The adhesion process, in both normal and pathological settings, follows a multi-step cascade that involves intercellular adhesion molecule-1 (ICAM-1), vascular cell adhesion molecule-1 (VCAM-1), and endothelial leukocyte adhesion molecule-1 (E-selectin) expression on endothelial cells (EC). The expression of these molecules and others on the vascular endothelium determines the efficiency with which leukocytes may adhere to the local vasculature and extravasate into the local tissue during the development of an inflammatory response. The local concentration of cytokines and growth factor participate in the modulation of the expression of these CAMs.

Tumor necrosis factor alpha (TNF-a), a potent proinflammatory cytokine, is a stimulator of all three CAMs on endothelial cells and may be involved in a wide variety of inflammatory responses, often resulting in a pathological outcome.

The potential of a polypeptide of the invention to mediate a suppression of TNF-a induced CAM expression can be examined. A modified ELISA assay which uses ECs as a solid phase absorbent is employed to measure the amount of CAM expression on TNF-a treated ECs when co-stimulated with a member of the FGF family of proteins.

To perform the experiment, human umbilical vein endothelial cell (HUVEC) cultures are obtained from pooled cord harvests and maintained in growth medium (EGM-2; Clonetics, San Diego, Calif.) supplemented with 10% FCS and 1% penicillin/streptomycin in a 37 degree C. humidified incubator containing 5% CO2. HUVECs are seeded in 96-well plates at concentrations of 1×104 cells/well in EGM medium at 37 degree C. for 18-24 hrs or until confluent. The monolayers are subsequently washed 3 times with a serum-free solution of RPMI-1640 supplemented with 100 U/ml penicillin and 100 mg/ml streptomycin, and treated with a given cytokine and/or growth factor(s) for 24 h at 37 degree C. Following incubation, the cells are then evaluated for CAM expression.

Human Umbilical Vein Endothelial cells (HUVECs) are grown in a standard 96 well plate to confluence. Growth medium is removed from the cells and replaced with 90 ul of 199 Medium (10% FBS). Samples for testing and positive or negative controls are added to the plate in triplicate (in 10 ul volumes). Plates are incubated at 37 degree C. for either 5 h (selectin and integrin expression) or 24 h (integrin expression only). Plates are aspirated to remove medium and 100 μl of 0.1% paraformaldehyde-PBS (with Ca++ and Mg++) is added to each well. Plates are held at 4° C. for 30 min.

Fixative is then removed from the wells and wells are washed 1× with PBS (+Ca,Mg)+0.5% BSA and drained. Do not allow the wells to dry. Add 10 μl of diluted primary antibody to the test and control wells. Anti-ICAM-1-Biotin, Anti-VCAM-1-Biotin and Anti-E-selectin-Biotin are used at a concentration of 10 μg/ml (1:10 dilution of 0.1 mg/ml stock antibody). Cells are incubated at 37° C. for 30 min. in a humidified environment. Wells are washed X3 with PBS (+Ca,Mg)+0.5% BSA.

Then add 20 μl of diluted ExtrAvidin-Alkaline Phosphatase (1:5,000 dilution) to each well and incubated at 37° C. for 30 min. Wells are washed X3 with PBS (+Ca,Mg)+0.5% BSA. 1 tablet of p-Nitrophenol Phosphate pNPP is dissolved in 5 ml of glycine buffer (pH 10.4). 100 μl of pNPP substrate in glycine buffer is added to each test well. Standard wells in triplicate are prepared from the working dilution of the ExtrAvidin-Alkaline Phosphatase in glycine buffer: 1:5,000 (100)>10-0.5>10-1 >10-1.5. 5 μl of each dilution is added to triplicate wells and the resulting AP content in each well is 5.50 ng, 1.74 ng, 0.55 ng, 0.18 ng. 100 μl of pNNP reagent must then be added to each of the standard wells. The plate must be incubated at 37° C. for 4h. A volume of 50 μl of 3M NaOH is added to all wells. The results are quantified on a plate reader at 405 nm. The background subtraction option is used on blank wells filled with glycine buffer only. The template is set up to indicate the concentration of AP-conjugate in each standard well [5.50 ng; 1.74 ng; 0.55 ng; 0.18 ng]. Results are indicated as amount of bound AP-conjugate in each sample.

From the foregoing, it is apparent that the invention includes a number of general uses tat can be expressed concisely as follows. The invention provides for the use of any of the ncletic acid seqments described above in the diagnosis or monitoring of diseases, such as cancer, inflammation, heart disease, diseases of the cardiovascular system, and infection by microorganisms. The invention further provides for the use of any of the nucleic acid segments in the manufacture of a medicament for the treatment or prophylaxis of such diseases. The invention further provides for the use of any of the DNA segments as a pharmaceutical.

### Example 35

Method of Assessing the Effect on Low Flow Ischemia in an Isolated Perfused Rat Heart Model by a Polypeptide of the Present Invention

Male Sprague-Dawley rats (350-450 grams) are fasted overnight and then anesthetized with sodium pentobarbital (30-40 mg/kg, ip). Following intubation by tracheotomy, the animals are ventilated with a rodent respirator (Model 683, Harvard Instruments, South Natick, Mass.) at a tidal volume of 4-5 ml delivered at 65-75 breaths/min and anticoagulated with sodium heparin (1000 IU/kg) administered via external jugular vein. A median thoracotomy may then be performed, the ribs are retracted, and the heart may then be exposed. The pericardium may be removed and the ascending aorta cleared of all connective tissue. A 2-0 silk suture may be placed around the base of the aorta in order to secure a perfusion cannula. The inferior vena cava may be then clamped and an incision may be made in the base of aorta. A custom steel cannula connected to a 3 way stopcock may be quickly inserted through the incision and then secured with the preplaced suture. Retrograde extracorporeal perfusion may be established with oxygenated (95% oxygen, 5% carbon dioxide, pH 7.4) Krebs-Henseleit solution comprised of (in mM) 1.25 calcium chloride, 112 sodium chloride, 25 sodium bicarbonate, 5 potassium chloride, 1 potassium biphosphate, 1.2 magnesium sulfate and 5.5 dextrose. The heart may be then transferred to a standard Langendorff perfusion apparatus [Doring et al., The isolated perfused warm-blooded heart according to Langendorff, 1st ed. March: Biomesstechnik-Verlag; 1988] where it may be perfused with oxygenated Krebs-Henseleit buffer warmed to 37 DEG C. and delivered at a constant perfusion pressure of 86 mm Hg. A water filed latex ballon may be fashioned from a latex finger cot (#55613-413, VWR Scientific, S. Plainfield, N.J.) and attached to a stainless steel cannula (model LL2, Hugo Sachs, March-Hugstetten, Germany) which may be then inserted into the left ventricle. The cannula may be attached to a pressure transducer (model P23, Gould Instruments, Valley View, Ohio) for the measurement of developed ventricular force. The heart may be then submerged in a water-jacketed (37 DEG C.) organ bath. Perfusate flow may be monitored with an extracorporeal electromagnetic flow probe (model MDL 1401, Skalar Instruments, Litchfield, Conn.). Hearts are allowed to beat at their intrinsic normal sinus rate. All data are continuously digitized at 250 Hz for subsequent analysis (Po-Neh-Mah Acquisition System, Gould Instruments, Valley View, Ohio). From the digitized data, steady state measurements for heart rate, perfusate flow and LV (left ventricular) developed pressure (LV systolic-LV end-diastolic pressure) are obtained during control, drug pretreatment, low flow and reperfusion. Hearts are prepared and assayed in quadruplicate.

### Ventricular Performance

Periodic load independent indices of myocardial performance are obtained as the mean slope of the linear portion of triplicate Frank-Starling (FS) curves [Schlant, Normal physiology of the cardiovascular system. In: Hurst JW, ed. The Heart, 4th ed. New York: McGraw-Hill; 1978: 71-100].

Similarly, the mean of the peak left ventricular developed pressures (LVDPmax) obtained during each discrete series of FS curves may be also recorded and meaned. FS curves are obtained by the inflation of the intraventricular balloon at a constant rate of 50 .mu.l/min with a programmable infusion/withdrawal pump (model 44, Harvard Apparatus, South Natick, Mass.). Balloon inflation may be discontinued at the onset of the descending limb of the FS curve, defined as that point where left ventricular developed pressure (LVDP) declined with further increases in balloon volume (preload). The balloon volume may be then removed at 300 .mu.l/min until LVDP may be undetectable (<2 mmHg). This process may be repeated until 3 reproducible curves are obtained.

### Preparation and Administration of Vector

Polynucleotides encoding polypeptides of the present invention may be cloned into an appropriate vector (referred to as “test vector”) as described herein or otherwise known in the art and administered in a pharmaceutically effective amount via infusion into the distal perfusion stream of each heart with a programmable infusion pump (model 22, Harvard Apparatus, South Natick, Mass.). Each pump may be controlled by a custom computer program which continuously monitored the perfusate flow in each heart, and dynamically adjusted the infusion rate of a test vector to maintain a constant DMSO concentration of 0.04%. Vehicle hearts are treated in an identical manner without said polynucleotides. Vectors may represent plasmids, viral vectors, etc., any of which comprising the encoding polynucleotide sequence of a polypeptide of the present invention, or fragment thereof.

### Experimental Protocol

Using this model, the vector comprising the encoding polynucleotide sequence of a polypeptide of the present invention, or fragment thereof may be compared to both vehicle and the selective angiotensin converting enzyme inhibitor fosinoprilat (free acid form of fosinopril). Test vector may be run in 20 hearts, vehicle in 21, and fosinoprilat in 19, for example.

The maximum dose of each vector/compound may be limited to the maximum no effect hemodynamic dose, assessed in normal hearts, in order to avoid the confounding effects of pharmacologically induced cardio-depression on ventricular performance.

Following a preliminary five minute equilibration period, control FS curves are performed in each heart and LVDPmax noted for each heart. Experimental preload (balloon volume) may be then adjusted to that unique balloon volume which produced 70% of LVDPmax in each heart. This volume may be then maintained as subsequently detailed. A five minute control period ensued once the specified preload had been achieved in all hearts. At this point infusion of either test vector, drug, or vehicle may commence and may be continued for the remainder of the experiment.

In order to avoid confounding inotropic drug effects, the dosage rationale for drug treatment during low flow ischemia may be to end with the highest concentration which did not affect steady state hemodynamics at normal perfusion pressure. Following a 5 minute control period, the drug may be administered as a continuous infusion for 10 minutes at normal perfusion (86 mmHg), and throughout 45 minutes of low flow ischemia (50 mmHg).

The slope of the Frank-Starling (FS) relationship may be employed as a load independent index of ventricular contractible function during control and low flow ischemia. All ES data are normalized and expressed as a percent of the control FS for each heart. Data for all like groups are pooled and are expressed as mean .+−.sem (standard error of the mean). All groups are compared by a one way analysis of variance. A p value of <0.05 may be considered significant.

Additional methods may be employed to determine the effect of the polynucleotides and polypeptides of the present invention on cardiovascular function, or to exemplify any function associated with said polynucleotides and polypeptides herein, such as for example, those methods described in U.S. Pat. Nos. 6,140,319; 6,248,729; International Publication No. WO0174348; International Publication No. WO0057883; and International Publication No. WO9965500.

One skilled in the art could easily modify the exemplified studies to test the activity of polynucleotides of the invention (e.g., gene therapy), agonists, and/or antagonists of polynucleotides or polypeptides of the invention.

### Example 36

Additional Methods of Genotyping the SNPs of the Present invention

The skilled artisan would acknowledge that there are a number of methods that may be employed for genotyping a SNP of the present invention, aside from the preferred methods described herein. The present invention encompasses the following non-limiting types of genotype assays: PCR-free genotyping methods, Single-step homogeneous methods, Homogeneous detection with fluorescence polarization, Pyrosequencing, “Tag” based DNA chip system, Bead-based methods, fluorescent dye chemistry, Mass spectrometry based genotyping assays, TaqMan genotype assays, Invader genotype assays, and microfluidic genotype assays, among others.

Specifically encompassed by the present invention are the following, non-limiting genotyping methods: Landegren, U., Nilsson, M. & Kwok, P. Genome Res 8, 769-776 (1998); Kwok, P., Pharmacogenomics 1, 95-100 (2000); Gut, I., Hum Mutat 17, 475-492 (2001); Whitcombe, D., Newton, C. & Little, S., Curr Opin Biotechnol 9, 602-608 (1998); Tillib, S. & Mirzabekov, A., Curr Opin Biotechnol 12, 53-58 (2001); Winzeler, E. et al., Science 281, 1194-1197 (1998); Lyamichev, V. et al., Nat Biotechnol 17, 292-296 (1999); Hall, J. et al., Proc Natl Acad Sci U S A 97, 8272-8277 (2000); Mein, C. et al., Genome Res 10, 333-343 (2000); Ohnishi, Y. et al., J Hum Genet 46, 471-477 (2001); Nilsson, M. et al., Science 265, 2085-2088 (1994); Baner, J., Nilsson, M., Mendel-Hartvig, M. & Landegren, U., Nucleic Acids Res 26, 5073-5078 (1998); Baner, J. et al., Curr Opin Biotechnol 12, 11-15 (2001); Hatch, A., Sano, T., Misasi, J. & Smith, C., Genet Anal 15, 35-40 (1999); Lizardi, P. et al., Nat Genet 19, 225-232 (1998); Zhong, X., Lizardi, P., Huang, X., Bray-Ward, P. & Ward, D., Proc Natl Acad Sci U S A 98, 3940-3945 (2001); Faruqi, F. et al. BMC Genomics 2, 4 (2001); Livak, K., Gnet Anal 14, 143-149 (1999); Marras, S., Kramer, F. & Tyagi, S., Genet Anal 14, 151-156 (1999); Ranade, K. et al., Genome Res 11, 1262-1268 (2001); Myakishev, M., Khripin, Y., Hu, S. & Hamer, D., Genome Re 11, 163-169 (2001); Beaudet, L., Bedard, J., Breton, B., Mercuri, R. & Budarf, M., Genome Res 11, 600-608 (2001); Chen, X., Levine, L. & P Y, K., Genome Res 9, 492-498 (1999); Gibson, N. et al., Clin Chem 43, 1336-1341 (1997); Latif, S., Bauer-Sardina, I., Ranade, K., Livak, K. & P Y, K., Genome Res 11, 436-440 (2001); Hsu, T., Law, S., Duan, S., Neri, B. & Kwok, P., Clin Chem 47, 1373-1377 (2001); Alderbom, A., Kristofferson, A. & Hammerling, U., Genome Res 10, 1249-1258 (2000); Ronaghi, M., Uhlen, M. & Nyren, P., Science 281, 363, 365 (1998); Ronaghi, M., Genome Res 11, 3-11 (2001); Pease, A. et al., Proc Natl Acad Sci U S A 91, 5022-5026 (1994); Southern, E., Maskos, U. & Elder, J., Genomics 13, 1008-1017 (1993); Wang, D. et al., Science 280, 1077-1082 (1998); Brown, P. & Botstein, D., Nat Genet 21, 33-37 (1999); Cargill, M. et al. Nat Genet 22, 231-238 (1999); Dong, S. et al., Genome Res 11, 1418-1424 (2001); Halushka, M. et al., Nat Genet 22, 239-247 (1999); Hacia, J., Nat Genet 21, 42-47 (1999); Lipshutz, R., Fodor, S., Gingeras, T. & Lockhart, D., Nat Genet 21, 20-24 (1999); Sapolsky, R. et al., Genet Anal 14, 187-192 (1999); Tsuchihashi, Z. & Brown, P., J Virol 68, 5863 (1994); Herschlag, D., J Biol Chem 270, 20871-20874 (1995); Head, S. et al., Nucleic Acids Res 25, 5065-5071 (1997); Nikiforov, T. et al., Nucleic Acids Res 22, 4167-4175 (1994); Syvanen, A. et al., Genomics 12, 590-595 (1992); Shumaker, J., Metspalu, A. & Caskey, C., Hum Mutat 7, 346-354 (1996); Lindroos, K., Liljedahl, U., Raitio, M. & Syvanen, A., Nucleic Acids Res 29, E69-9 (2001); Lindblad-Toh, K. et al., Nat Genet 24, 381-386 (2000); Pastinen, T. et al., Genome Res 10, 1031-1042 (2000); Fan, J. et al., Genome Res 10, 853-860 (2000); Hirschhorn, J. et al., Proc Natl Acad Sci U S A 97, 12164-12169 (2000); Bouchie, A., Nat Biotechnol 19, 704 (2001); Hensel, M. et al., Science 269, 400-403 (1995); Shoemaker, D., Lashkari, D., Morris, D., Mittmann, M. & Davis, R. Nat Genet 14, 450-456 (1996); Gerry, N. et al., J Mol Biol 292, 251-262 (1999); Ladner, D. et al., Lab Invest 81, 1079-1086 (2001); lannone, M. et al. Cytometry 39, 131-140 (2000); Fulton, R., McDade, R., Smith, P., Kienker, L. & Kettman, J. J., Clin Chem 43, 1749-1756 (1997); Armstrong, B., Stewart, M. & Mazumder, A., Cytometry 40, 102-108 (2000); Cai, H. et al., Genomics 69, 395 (2000); Chen, J. et al., Genome Res 10, 549-557 (2000); Ye, F. et al. Hum Mutat 17, 305-316 (2001); Michael, K., Taylor, L., Schultz, S. & Walt, D., Anal Chem 70, 1242-1248 (1998); Steemers, F., Ferguson, J. & Walt, D., Nat Biotechnol 18, 91-94 (2000); Chan, W. & Nie, S., Science 281, 2016-2018 (1998); Han, M., Gao, X., Su, J. & Nie, S., Nat Biotechnol 19, 631-635 (2001); Griffin, T. & Smith, L., Trends Biotechnol 18, 77-84 (2000); Jackson, P., Scholl, P. & Groopman, J., Mol Med Today 6, 271-276 (2000); Haff, L. & Smirnov, I., Genome Res 7, 378-388 (1997); Ross, P., Hall, L., Smirnov, I. & Haff, L., Nat Biotechnol 16, 1347-1351 (1998); Bray, M., Boerwinkle, E. & Doris, P. Hum Mutat 17, 296-304 (2001); Sauer, S. et al., Nucleic Acids Res 28, E13 (2000); Sauer, S. et al., Nucleic Acids Res 28, E100(2000); Sun, X., Ding, H., Hung, K. & Guo, B., Nucleic Acids Res 28, E68 (2000); Tang, K. et al., Proc Natl Acad Sci U S A 91, 10016-10020 (1999); Li, J. et al., Electrophoresis 20, 1258-1265 (1999); Little, D., Braun, A., O'Donnell, M. & Koster, H., Nat Med 3, 1413-1416 (1997); Little, D. et al. Anal Chem 69, 4540-4546 (1997); Griffin, T., Tang, W. & Smith, L., Nat Biotechnol 15, 1368-1372 (1997); Ross, P., Lee, K. & Belgrader, P., Anal Chem 69, 4197-4202 (1997); Jiang-Baucom, P., Girard, J., Butler, J. & Belgrader, P., Anal Chem 69, 4894-4898 (1997); Griffin, T., Hall, J., Prudent, J. & Smith, L., Proc Natl Acad Sci U S A 96, 6301-6306 (1999); Kokoris, M. et al., Mol Diagn 5, 329-340 (2000); Jurinke, C., van den Boom, D., Cantor, C. & Koster, H. (2001); and/or Taranenko, N. et al., Genet Anal 13, 87-94 (1996).

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the Sequence Listing submitted herewith and the corresponding computer readable form are both incorporated herein by reference in their entireties.

While this invention has been particularly shown and described with references to preferred embodiments thereof, it will be understood by those skilled in the art that various changes in form and details may be made therein without departing from the scope of the invention encompassed by the appended claims.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the Sequence Listing submitted herewith and the corresponding computer readable form are both incorporated herein by reference in their entireties.

While this invention has been particularly shown and described with references to preferred embodiments thereof, it will be understood by those skilled in the art that various changes in form and details may be made therein without departing from the scope of the invention encompassed by the appended claims.

TABLE I

| | | | | REFSEQ_FLANK_REF | | | | | | |
|---|---|---|---|---|---|---|---|---|---|---|
| | | | | SEQ | | | | | | |
| GENE_DESCR. | HGNC_ID | SNP_ID | REFSEQ_FLANK_REF | ID NO: | REF_NT | EXON | MUTATION_TYPE | REV_COMP | CDNA_SEQ_ID | CDNA_SEQ_POS |
| C1,S | C1S | AELLLs1 | TATTGTTTTTTTGTFFGTTTGTTFFCAAG | 1 | T | 5FLANK | Non-CDS | 1 | NM_001 | |
| subcomponent | | | TTTGGGACTAAAA | | | | | 734.1 | | |
| C1,S | C1S | AE111s2 | CCTGCAGAGGGAGCGTCAAGGCCCTG | 2 | G | Exon1 | Non-CDS | 1 | NM_001 | 32 |
| subcomponent | | | TGCTGCTGTCCCTGG | | | | | | 734.1 | |
| C1,S | C1S | AE111s13 | GGCGGGAGGATTGCTTTGAGCCCAGGA | 3 | C | Intron2 | Non-CDS | 1 | NM_001 | |
| subcomponent | | | CTTTGAGACCAGCCT | | | | | | 734.1 | |
| C1,S | C1S | AE111s15 | GCACAAAAAGAATAGAGATGGAAGA | 4 | G | Intron3 | Non-CDS | 1 | NM_001 | |
| subcomponent | | | CTAGGGCTAAGGTAGC | | | | | | 734.1 | |
| C1,S | C1S | AE111s17 | AGACTTTTCCAATGAAGAGCGTTTTAC | 5 | G | Exon4 | Missense | 1 | NM_001 | 558 |
| subcomponent | | | GGGGTTTGCTGCAT | | | | | | 734.1 | |
| C1,S | C1S | AE111s19 | TCTTACCCTTATGGTTTTGGATTTAAC | 6 | A | Intron4 | Non-CDS | 1 | NM_001 | |
| subcomponent | | | CTCATTCTCCCTTC | | | | | | 734.1 | |
| C1,S | C1S | AE111s18 | GTCCCTTGTAGCCACTTCTGCAACAAT | 7 | C | Exon5 | Silent | 1 | NM_001 | 643 |
| subcomponent | | | TTTCATTGGTGGTTA | | | | | | 734.1 | |
| C1,S | C1S | AE111s20 | GGATTCCCTGGGCCTCTAAATATTGAA | 8 | T | Exon7 | Missense | 1 | NM_001 | 982 |
| subcomponent | | | ACCAAGAGTAATGC | | | | | | 734.1 | |
| C1,S | C1S | AE111s21 | CAATTCTGTTTGGGAGCCTGCGAAGG | 9 | C | Exon8 | Missense | 1 | NM_001 | 1122 |
| subcomponent | | | CAAAATATGTCTTTA | | | | | | 734.1 | |
| C1,S | C1S | AE111s22 | AGCCTGCGAAGGCAAAATATGTCTTT | 10 | G | Exon8 | Missense | 1 | NM_001 | 1136 |
| subcomponent | | | AGAGATGTGGTGCAG | | | | | | 734.1 | |
| C1,S | C1S | AE111s5 | CCACAGAGAGGCTGGTGTGGGGAGGT | 11 | G | Intron9 | Non-CDS | 1 | NM_001 | |
| subcomponent | | | TCATCCCAGGTGTGC | | | | | | 734.1 | |
| C1,S | C1S | AE111s6 | TTGGGGAGAGATGATAGCCATGGGTG | 12 | T | Intron1 | Non-CDS | 1 | NM_001 | |
| subcomponent | | | ACTGGGAGTCACCTT | | | | | | 734.1 | |
| C1,S | C1S | AE111s8 | ACCTCTTTCCGACTACAACCTCATGGAT | 13 | C | Exon12 | Silent | 1 | NM_001 | 1879 |
| subcomponent | | | GGGGACCTGGGACT | | | | | | 734.1 | |
| alanyl | ANPEP | AE112s57 | TCCCAGACTCCACGGTGCTCCGCATGC | 14 | C | 3FLANK | Non-CDS | 0 | NM_001 | |
| aminopeptidase | | | ACCCAGCTTCCCCC | | | | | | 150.1 | |
| alanyl | ANPEP | AE112s55 | GGCCCTGGAGCTGGGCTTCCCTGAGA | 15 | C | Exon20 | Non-CDS | 0 | NM_001 | 3296 |
| aminopeptidase | | | TCAGCCCCAGGGCAC | | | | | | 150.1 | |
| alanyl | ANPEP | AE112s56 | CAGCCTGGGTCATCAGGAACTAGACT | 16 | T | Exon20 | Non-CDS | 0 | NM_001 | 3185 |
| aminopeptidase | | | GGCTCACAGGCAGAG | | | | | | 150.1 | |
| alanyl | ANPEP | AE112s58 | ATGGAGGCCCTGCACCAGCCGCTGGG | 17 | G | Exon20 | Non-CDS | 0 | NM_001 | 3084 |
| aminopeptidase | | | ATGGACACATGTGGG | | | | | | 150.1 | |
| alanyl | ANPEP | AE112s44 | CTGGCTACTCGGCTTCCTCTGTAAATG | 18 | G | Intron19 | Non-CDS | 0 | NM_001 | |
| aminopeptidase | | | AGGAAGGCCTGGCG | | | | | | 150.1 | |
| alanyl | ANPEP | AE112s46 | TGTGCTCCCTCAGGGCCACGTGGGAG | 19 | T | Intron19 | Non-CDS | 0 | NM_001 | |
| aminopeptidase | | | AAGAATGGAGTGCCC | | | | | | 150.1 | |
| alanyl | ANPEP | AE112s42 | AATGGAGTGCCCCTTTGGCCTGGGAA | 20 | T | Intron19 | Non-CDS | 0 | NM_001 | |
| aminopeptidase | | | GGAAGTAGGCAGAGC | | | | | | 150.1 | |
| alanyl | ANPEP | AE112s43 | CCCCTTTGGCCTGGGAAGGAAGTAGG | 21 | A | Intron19 | Non-CDS | 0 | NM_001 | |
| aminopeptidase | | | CAGAGCCCAGGTCTG | | | | | | 150.1 | |
| alanyl | ANPEP | AE112s41 | TCACACCTACTGGGTAGGGAGCCATG | 22 | G | Intron18 | Non-CDS | 0 | NM_001 | |
| aminopeptidase | | | ATTATAGAGGAGATG | | | | | | 150.1 | |
| alanyl | ANPEP | AE112s40 | CCCCCCGGGGCCCCAGCCTCGGCGCT | 23 | G | Intron18 | Non-CDS | 0 | NM_001 | |
| aminopeptidase | | | CGCTGTCCCTGACAC | | | | | | 150.1 | |
| alanyl | ANPEP | AE112s36 | TCCCAGACCAGACCTTGCCCAATGAC | 24 | A | Exon18 | Silent | 0 | NM_001 | 2733 |
| aminopeptidase | | | GTTGTTGGTAATGCT | | | | | | 150.1 | |
| alanyl | ANPEP | AE112s38 | CGGATTAAGTCCGGGTTCAGGGTGTA | 25 | G | Exon18 | Silent | 0 | NM_001 | 2664 |
| aminopeptidase | | | GCTCAGGTACCTAAG | | | | | | 150.1 | |
| alanyl | ANPEP | AE112s35 | CTAAGAGGGCCAGATGCTGTCTCAGC | 26 | C | Intron17 | Non-CDS | 0 | NM_001 | |
| aminopeptidase | | | TACTGCTAATTCAGG | | | | | | 150.1 | |
| alanyl | ANPEP | AE112s29 | CTCTCGCAGTCCCACCCTGCGCCAAG | 27 | G | Intron17 | Non-CDS | 0 | NM_001 | |
| aminopeptidase | | | ACTCACCTGTTCAGG | | | | | | 150.1 | |
| alanyl | ANPEP | AE112s33 | TTTCGGAACTGCTCCCAGGCGAAGTC | 28 | G | Exon17 | Silent | 0 | NM_001 | 2553 |
| aminopeptidase | | | CCACTCCTCCTCCCC | | | | | | 150.1 | |
| alanyl | ANPEP | AE112s32 | CCTCCCCGCCCTGGGCGATAGCGTTGC | 29 | G | Exon17 | Missense | 0 | NM_001 | 2519 |
| aminopeptidase | | | AGTAGACGGTGGAC | | | | | | 150.1 | |
| alanyl | ANPEP | AE112s30 | GCGATAGCGTTGCAGTAGACGGTGGA | 30 | G | Exon17 | Silent | 0 | NM_001 | 2505 |
| aminopeptidase | | | CCGCAGGTTGGGGTG | | | | | | 150.1 | |
| alanyl | ANPEP | AE112s26 | TAGTTCTGGGGAAAAGAAAATATGAA | 31 | T | Intron14 | Non-CDS | 0 | NM_001 | |
| aminopeptidase | | | TCTCATCAAAGACTC | | | | | | 150.1 | |
| alanyl | ANPEP | AE112s22 | GTGGGGACCCAGGGAGGGACGTCCAG | 32 | G | Intron13 | Non-CDS | 0 | NM_001 | |
| aminopeptidase | | | GGAGCACAGGAGCTC | | | | | | 150.1 | |
| alanyl | ANPEP | AE112s23 | GAGCTCAGGGCACAGCACGTGGCATA | 33 | G | Intron13 | Non-CDS | 0 | NM_001 | |
| aminopeptidase | | | TGGGAAGGGCAGCAG | | | | | | 150.1 | |
| alanyl | ANPEP | AE112s21 | GAAGTCACGAGCTTCTGCAGCTGAGC | 34 | C | Intron13 | Non-CDS | 0 | NM_001 | |
| aminopeptidase | | | CAGGCAGCGGAGCAC | | | | | | 150.1 | |
| alanyl | ANPEP | AE112s17 | TGTTGAATGGAATGGCCCATCACAGC | 35 | C | Intron10 | Non-CDS | 0 | NM_001 | |
| aminopeptidase | | | CCTCAGAACATGGGC | | | | | | 150.1 | |
| alanyl | ANPEP | AE112s18 | GCTGGATTACCTCTTACATCTATCAGC | 36 | T | Exon11 | Missense | 0 | NM_001 | 1929 |
| aminopeptidase | | | CAGTAGTCCTGCTG | | | | | | 150.1 | |
| alanyl | ANPEP | AE112s19 | CTGGATTACCTCTTACATCTATCAGCC | 37 | A | Exon11 | Missense | 0 | NM_001 | 1928 |
| aminopeptidase | | | AGTAGTCCTGCTGC | | | | | | 150.1 | |
| alanyl | ANPEP | AE112s71 | CCCTCCAGGCCAAGTCCCCACCTCCTT | 38 | C | Intron7 | Non-CDS | 0 | NM_001 | |
| aminopeptidase | | | CCCCGTGCCCCACG | | | | | | 150.1 | |
| alanyl | ANPEP | AE112s72 | ACCTCCTTCCCCGTGCCCCACGAGGA | 39 | C | Intron7 | Non-CDS | 0 | NM_001 | |
| aminopeptidase | | | GCGGGCTGCACCTTG | | | | | | 150.1 | |
| alanyl | ANPEP | AE112s69 | CATGCCCCCCGCACCAGACCCCTGGG | 40 | C | Intron6 | Non-CDS | 0 | NM_001 | |
| aminopeptidase | | | CAGCTGGCTTACCAA | | | | | | 150.1 | |
| alanyl | ANPEP | AE112s68 | CTGGAGGAGGACAGGGGGTCGAACA | 41 | G | Exon5 | Silent | 0 | NM_001 | 1227 |
| aminopeptidase | | | GCAGGGAGTTCTCCCG | | | | | | 150.1 | |
| alanyl | ANPEP | AE112s64 | TCTGGTCTGGGGAGGCGATGCCATTG | 42 | C | Intron4 | Non-CDS | 0 | NM_001 | |
| aminopeptidase | | | GCAGGATGAACTCCG | | | | | | 150.1 | |
| alanyl | ANPEP | AE112s63 | AAGAAGTTAAGGATGGGGCCCGTCAC | 43 | C | Exon4 | Silent | 0 | NM_001 | 1083 |
| aminopeptidase | | | GTTCAGGGCATAATC | | | | | | 150.1 | |
| alanyl | ANPEP | AE112s62 | AGGATGGGGCCCGTCACGTTCAGGGC | 44 | C | Exon4 | Silent | 0 | NM_001 | 1074 |
| aminopeptidase | | | ATAATCGCCGTGGCC | | | | | | 150.1 | |
| alanyl | ANPEP | AE112s61 | GGGCATAATCGCCGTGGCCCGCCGCA | 45 | G | Exon4 | Missense | 0 | NM_001 | 1052 |
| aminopeptidase | | | ATGGCACTGGGCCGG | | | | | | 150.1 | |
| alanyl | ANPEP | AE112s65 | CAGGTGGGCTGGGTCCCAGGGCCCAG | 46 | G | Intron3 | Non-CDS | 0 | NM_001 | |
| aminopeptidase | | | TGGGCTGGGGGGATC | | | | | | 150.1 | |
| alanyl | ANPEP | AE112s53 | CCTGGCCCTGTGGCCGCAGGCAGGGC | 47 | C | Intron2 | Non-CDS | 0 | NM_001 | |
| aminopeptidase | | | CCACTCACCTTTGGG | | | | | | 150.1 | |
| alanyl | ANPEP | AE112s52 | TCTGCAGCCTGCATCTGTGTAGTGGCC | 48 | A | Exon2 | Silent | 0 | NM_001 | 747 |
| aminopeptidase | | | ACCACCCTGCCCCA | | | | | | 150.1 | |
| alanyl | ANPEP | AE112s13 | AGCACCTCGGATCCACCCCACCGGGC | 49 | C | Intron1 | Non-CDS | 0 | NM_001 | |
| aminopeptidase | | | AGCCCAGCCGGAACT | | | | | | 150.1 | |
| alanyl | ANPEP | AE112s15 | TTGCTGTGGATGATGATGACGTCAGT | 50 | G | Exon1 | Silent | 0 | NM_001 | 474 |
| aminopeptidase | | | GGCCTCCTTGCAGGT | | | | | | 150.1 | |
| alanyl | ANPEP | AE112s1 | GGCTCCAACAGGCGAAGGTCACTGGA | 51 | A | 5FLANK | Non-CDS | 0 | NM_001 | |
| aminopeptidase | | | CTGGGCAGGGGCACG | | | | | | 150.1 | |
| alanyl | ANPEP | AE112s5 | AGTTCCTCCAGGTTCCCCTCCCTGCCG | 52 | C | 5FLANK | Non-CDS | 0 | NM_001 | |
| aminopeptidase | | | CTTCTTGCCAAATA | | | | | | 150.1 | |
| alanyl | ANPEP | AE112s4 | TAAAATGAAATGAGTTGTTTTGCTTTT | 53 | T | 5FLANK | Non-CDS | 0 | NM_001 | |
| aminopeptidase | | | TTTGCTGAAGGCTT | | | | | | 150.1 | |
| alanyl | ANPEP | AE112s3 | CAGGTTTGTTGAACAGATTTAGTGAG | 54 | A | 5FLANK | Non-CDS | 0 | NM_001 | |
| aminopeptidase | | | AAAACATATTAAACA | | | | | | 150.1 | |
| alanyl | ANPEP | AE112s2 | GTGAGAAAACATATTAAACACCAAAT | 55 | C | 5FLANK | Non-CDS | 0 | NM_001 | |
| aminopeptidase | | | AGTAGAATGATTAAA | | | | | | 150.1 | |
| alanyl | ANPEP | AE112s9 | AGGCCGAGGTGGGTGGATCACGAGGT | 56 | C | 5FLANK | Non-CDS | 0 | NM_001 | |
| aminopeptidase | | | TAGGAGTTCAAGACC | | | | | | 150.1 | |
| alanyl | ANPEP | AE112s6 | AGTTGGGCGTGGTGGCGGGCGTCTGT | 57 | G | 5FLANK | Non-CDS | 0 | NM_001 | |
| aminopeptidase | | | AATCCCAGCTACTTG | | | | | | 150.1 | |
| alanyl | ANPEP | AE112s7 | GGCAGAGAATTGCTTGAATCCGGGAG | 58 | C | 5FLANK | Non-CDS | 0 | NM_001 | |
| aminopeptidase | | | GCAGAGATTGCAGTG | | | | | | 150.1 | |
| alanyl | ANPEP | AE112s11 | AITGCAGTGAGCTGAGATCGCACCAC | 59 | C | 5FLANK | Non-CDS | 0 | NM_001 | |
| aminopeptidase | | | TGCACTCCAGCCTGG | | | | | | 150.1 | |
| alanyl | ANPEP | AE112s12 | TTGCAGTGAGCTGAGATCGCACCACT | 60 | A | 5FLANK | Non-CDS | 0 | NM_001 | |
| aminopeptidase | | | GCACTCCAGCCTGGG | | | | | | 150.1 | |
| meprin A, beta | MEP1B | AE113s4 | GTGGTGTGATCTCGGCTCACCGCAAC | 61 | C | 5FLANK | Non-CDS | 1 | NM_005 | |
| | | | CTCTGCCTCCCAGGT | | | | | | 925.1 | |
| meprin A, beta | MEP1B | AE113s3 | AGCTGGGATTACAGACATGCACCACC | 62 | A | 5FLANK | Non-CDS | 1 | NM_005 | |
| | | | ACACCTGGCTAATTT | | | | | | 925.1 | |
| meprin A, beta | MEP1B | AE113s2 | TCTAGTAGAGATGGGGTTTCACTGTGT | 63 | A | 5FLANK | Non-CDS | 1 | NM_005 | |
| | | | TGGCCAGGTTGGGC | | | | | | 925.1 | |
| meprin A, beta | MEP1B | AE113s1 | GTTGGCCAGGTTGGGCTTGAACTCCC | 64 | A | 5FLANK | Non-CDS | 1 | NM_005 | |
| | | | GACCTCAGGTGGTCC | | | | | | 925.1 | |
| meprin A, beta | MEP1B | AE113s5 | ATAGGCACTCAATAATTTTTATTAAAT | 65 | A | 5FLANK | Non-CDS | 1 | NM_005 | |
| | | | GAGTGAATGATAAA | | | | | | 925.1 | |
| meprin A, beta | MEP1B | AE113s34 | TTGGGACTGGATCTTTTTGAGGGTGAC | 66 | G | Exon3 | Silent | 1 | NM_005 | 196 |
| | | | ATCAGACTTGATAG | | | | | | 925.1 | |
| meprin A, beta | MEP1B | AE113s37 | TTCAAAGGGTGGGCTTATATAGTCTTT | 67 | A | Intron5 | Non-CDS | 1 | NM_005 | |
| | | | AAGGTTTTGTTTGG | | | | | | 925.1 | |
| meprin A, beta | MEP1B | AE113s38 | GCTGGAGAAACAAACTATATATCAGT | 68 | A | Exon6 | Missense | 1 | NM_005 | 394 |
| | | | GTTCAAGGGCAGTGG | | | | | | 925.1 | |
| meprin A, beta | MEP1B | AE113s39 | CTGCCCTGAGACCTCAGATTGTAACAT | 69 | G | Intron6 | Non-CDS | 1 | NM_005 | |
| | | | TCTCTTCCCCCTTT | | | | | | 925.1 | |
| meprin A, beta | MEP1B | AE113s41 | ACATTTCTCTTTTTTTCCTATGTTTTTA | 70 | T | Intron7 | Non-CDS | 1 | NM_005 | |
| | | | GTTAAGGACTGAA | | | | | | 925.1 | |
| meprin A, beta | MEP1B | AE113s40 | TTTAGTTAAGGACTGAATTTCTAAGCA | 71 | C | Intron7 | Non-CDS | 1 | NM_005 | |
| | | | TGTGTCCTCTCTTG | | | | | | 925.1 | |
| meprin A, beta | MEP1B | AE113s43 | TCCTTGAGTTTTATGGACTCGTGCAGT | 72 | G | Exon9 | Silent | 1 | NM_005 | 838 |
| | | | TTTGAACTGGAAAA | | | | | | 925.1 | |
| meprin A, beta | MEP1B | AE113s42 | TAATGATTATTCATTAGGTTACTACTG | 73 | A | Intron9 | Non-CDS | 1 | NM_005 | |
| | | | AGAAGTAGGCCTTG | | | | | | 925.1 | |
| meprin A, beta | MEP1B | AE113s7 | CTCTCAATTCTGCTTTTCTTTAACAGG | 74 | T | Intron9 | Non-CDS | 1 | NM_005 | |
| | | | TTCTGGTTTCTTCA | | | | | | 925.1 | |
| meprin A, beta | MEP1B | AE113s6 | TAAATGTGGGGGCCACAGCAGTGCTG | 75 | G | Exon10 | Missense | 1 | NM_005 | 1022 |
| | | | GAAAGTAGAACGCTG | | | | | | 925.1 | |
| meprin A, beta | MEP1B | AE113s9 | TGTTTGAAGGACGCAAAGGCTCTGGT | 76 | T | Exon10 | Missense | 1 | NM_005 | 1268 |
| | | | GCATCACTGGGTGGT | | | | | | 925.1 | |
| meprin A, beta | MEP1B | AE113s8 | ATTGATGACATCAATCTTTCGGAAAC | 77 | 6 | Exon10 | Silent | 1 | NM_005 | 1315 |
| | | | ACGGTGCCCTCATCA | | | | | | 925.1 | |
| meprin A, beta | MEP1B | AE113s18 | AACCAGTGCCTTTTATAACCCACGAAA | 78 | A | Exon12 | Missense | 1 | NM_005 | 1740 |
| | | | GGCTGAAAAGCAGAG | | | | | | 925.1 | |
| meprin A, beta | MEP1B | AE113s14 | ACCAGTGCCTTTATAACCCACGAAAG | 79 | C | Exon12 | Silent | 1 | NM_005 | 1741 |
| | | | GCTGAAAAGCAGAGA | | | | | | 925.1 | |
| meprin A, beta | MEP1B | AE113s16 | TTTATATAAACTAGTTTTTTTTTGTTGT | 80 | T | Intron12 | Non-CDS | 1 | NM_005 | |
| | | | GGCCACTGATTAT | | | | | | 925.1 | |
| meprin A, beta | MEP1B | AE113s21 | TTCCACTTTTAGATGTATTATATAGAG | 81 | T | Intron12 | Non-CDS | 1 | NM_005 | |
| | | | ATGTGGGGGGAATG | | | | | | 925.1 | |
| meprin A, beta | MEP1B | AE113s22 | ATCAAATGATTGTTAAACAAAAGCTG | 82 | A | Intron12 | Non-CDS | 1 | NM_005 | |
| | | | ATITCCATCCACACT | | | | | | 925.1 | |
| meprin A, beta | MEP1B | AE113s24 | AAGAGAGGCTCCACCCGAGACACCAT | 83 | C | Exon14 | Silent | 1 | NM_005 | 1999 |
| | | | AGTCATTGCTGTTTC | | | | | | 925.1 | |
| meprin A, beta | MEP1B | AE113s25 | AAATCGACCAAATTTGACTCCGCAAA | 84 | C | Exon14 | Missense | 1 | NM_005 | 2130 |
| | | | ATGTAAGTTGAGGCT | | | | | | 925.1 | |
| meprin A, beta | MEP1B | AE113s26 | GGCTGATGTTTGATTATTCATAACCTA | 85 | T | Intron14 | Non-CDS | 1 | NM_005 | |
| | | | TTGGTGAAATCTTA | | | | | | 925.1 | |
| meprin A, beta | MEP1B | AE113s31 | CTTCATTTAAAGACCAGATCATTTTAT | 86 | A | Intron14 | Non-CDS | 1 | NM_005 | |
| | | | TATGATTCATTTAA | | | | | | 925.1 | |
| meprin A, beta | MEP1B | AE113s30 | AAAGTGGATATTTTTCTGTAAATAGCT | 87 | A | Exon15 | Non-CDS | 1 | NM_005 | 2265 |
| | | | GGAAATATTATAAA | | | | | | 925.1 | |
| Aminopeptidase | XPNPEPL | AE114s30 | TTCCCAATCACATCCCTGCAGGTCAGG | 88 | G | Exon19 | Silent | 0 | NM_006 | 1773 |
| P-like | | | TGGTAATTGTTGAG | | | | | | 523.1 | |
| Aminopeptidase | XPNPEPL | AE114s31 | CACAAGCTGAGAGAAAAGGTGTGCAA | 89 | G | Intron18 | Non-CDS | 0 | NM_006 | |
| P-like | | | CACCCTTTCTCCTTA | | | | | | 523.1 | |
| Aminopeptidase | XPNPEPL | AE114s29 | ATTAAAAAATACAAAACAACGAATAG | 90 | G | Intron16 | Non-CDS | 0 | NM_006 | |
| P-like | | | CTTTCAAAAAGGCTC | | | | | | 523.1 | |
| Aminopeptidase | XPNPEPL | AE114s28 | AAGAAGGTGACCTGAAAGACATAAAG | 91 | A | Intron15 | Non-CDS | 0 | NM_006 | |
| P-like | | | AGCCACTTAATTGTA | | | | | | 523.1 | |
| Aminopeptidase | XPNPEPL | AE114s26 | TGCAGAATGGGGACAGATGTCTAATC | 92 | C | Intron14 | Non-CDS | 0 | NM_006 | |
| P-like | | | CTGTAGTTTTCTGGT | | | | | | 523.1 | |
| Aminopeptidase | XPNPEPL | AE114s25 | AGGAGCAATTTTACTACCCCACATGT | 93 | A | Intron14 | Non-CDS | 0 | NM_006 | |
| P-like | | | AATTTTTACTTCTTT | | | | | | 523.1 | |
| Aminopeptidase | XPNPEPL | AE114s24 | GAGCAATTTTACTACCCCACATGTAAT | 94 | A | Intron14 | Non-CDS | 0 | NM_006 | |
| P-like | | | TTTTACTTCTTTAT | | | | | | 523.1 | |
| Aminopeptidase | XPNPEPL | AE114s23 | TATCAAACAGTCTTTTGTGAGAACAG | 95 | G | Intron13 | Non-CDS | 0 | NM_006 | |
| P-like | | | AGTCACAACTGGGCT | | | | | | 523.1 | |
| Aminopeptidase | XPNPEPL | AE114s22 | ACCAGCTGAGTGCTCTCAGCTGGAGC | 96 | T | Intron13 | Non-CDS | 0 | NM_006 | |
| P-like | | | TCAGCTGTCCACCTTI | | | | | | 523.1 | |
| Aminopeptidase | XPNPEPL | AE114s18 | AGCAGCAAGGCCAGGCAGCATGCTCC | 97 | T | Intron12 | Non-CDS | 0 | NM_006 | |
| P-like | | | TCCGCATGCCTTACG | | | | | | 523.1 | |
| Aminopeptidase | XPNPEPL | AE114s14 | CTGCCTGTTGCCTGTAACAGAAAGAC | 98 | A | Intron11 | Non-CDS | 0 | NM_006 | |
| P-like | | | AGAAAGCAGAGTGGC | | | | | | 523.1 | |
| Aminopeptidase | XPNPEPL | AE114s13 | CACGAGGCTCCTGGGTCCCCCCAAAT | 99 | C | Intron11 | Non-CDS | 0 | NM_006 | |
| P-like | | | GGATCCTTACCTGCG | | | | | | 523.1 | |
| Aminopeptidase | XPNPEPL | AE114s16 | AAAGCTCACTTTTTCCTCAATTGCTTC | 100 | T | Intron10 | Non-CDS | 0 | NM_006 | |
| P-like | | | ATCCCAACTGACCA | | | | | | 523.1 | |
| Aminopeptidase | XPNPEPL | AE114s12 | GCATCCACTCTATTATGGAACTTTTCT | 101 | C | Intron10 | Non-CDS | 0 | NM_006 | |
| P-like | | | TCATTCTTGTTACT | | | | | | 523.1 | |
| Aminopeptidase | XPNPEPL | AE114s45 | GGCTGAGGCTCAGAGAGATTCTAACC | 102 | C | Intron9 | Non-CDS | 0 | NM_006 | |
| P-like | | | TTGTGCCAGGCTCAA | | | | | | 523.1 | |
| Aminopeptidase | XPNPEPL | AE114s44 | CGGTGGTCCTAGAGGCAAAGGGCAGT | 103 | G | Intron8 | Non-CDS | 0 | NM_006 | |
| P-like | | | AGGGTGGGAAATCAA | | | | | | 523.1 | |
| Aminopeptidase | XPNPEPL | AE114s43 | GAGCCTGCAGATGGAGGAGAGGTGGG | 104 | G | Intron7 | Non-CDS | 0 | NM_006 | |
| P-like | | | TGGCAGAAAAAGGAG | | | | | | 523.1 | |
| Aminopeptidase | XPNPEPL | AE114s42 | AATATCAGGCAGTGATGGGTGGGCCA | 105 | G | Intron7 | Non-CDS | 0 | NM_006 | |
| P-like | | | GGACCAGAAAGGGCA | | | | | | 523.1 | |
| Aminopeptidase | XPNPEPL | AE114s41 | CAGCCCTGGTCCCAGAAAGCAGTAGG | 106 | A | Intron5 | Non-CDS | 0 | NM_006 | |
| P-like | | | AACCCAGTGAAAGAA | | | | | | 523.1 | |
| Aminopeptidase | XPNPEPL | AE114s35 | TCGACTTGGATTCGGAGGACGAGGCT | 107 | G | Intron5 | Non-CDS | 0 | NM_006 | |
| P-like | | | GGAGACAATAGCAAG | | | | | | 523.1 | |
| Aminopeptidase | XPNPEPL | AE114s34 | CTTGGATTCGGAGGACGAGGCTGGAG | 108 | C | Intron5 | Non-CDS | 0 | NM_006 | |
| P-like | | | ACAATAGCAAGAGGT | | | | | | 523.1 | |
| Aminopeptidase | XPNPEPL | AE114s36 | ACGCCCATTGCAGCAGGGCAGGGAGC | 109 | G | Intron4 | Non-CDS | 0 | NM_006 | |
| P-like | | | AGGGCCTTGAAAGTC | | | | | | 523.1 | |
| Aminopeptidase | XPNPEPL | AE114s38 | GCCCATTGCAGCAGGGCAGGGAGCAG | 110 | G | Intron4 | Non-CDS | 0 | NM_006 | |
| P-like | | | GGCCTTGAAAGTCCA | | | | | | 523.1 | |
| Aminopeptidase | XPNPEPL | AE114s33 | GCCTGGAGAAAGTAGCGCCCGTCAGT | 111 | G | Exon3 | Silent | 0 | NM_006 | 225 |
| P-like | | | CCACATGGCTGCATG | | | | | | 523.1 | |
| Aminopeptidase | XPNPEPL | AE114s32 | GACTGTTCTGCCTGCTTAGCCTTGTGC | 112 | C | Intron1 | Non-CDS | 0 | NM_006 | |
| P-like | | | TAGGCTCAGCGGGG | | | | | | 523.1 | |
| Aminopeptidase | XPNPEPL | AE114s5 | ATTTCAAAACCAGCCTGCCCCCAGCA | 113 | C | 5FLANK | Non-CDS | 0 | NM_006 | |
| P-like | | | ATTCAACGTCCAGTT | | | | | | 523.1 | |
| Aminopeptidase | XPNPEPL | AE114s3 | TAGGTGTTTATCTTTGTTTATAGTAGA | 114 | T | 5FLANK | Non-CDS | 0 | NM_006 | |
| P-like | | | AAAAAGCAGGAGAA | | | | | | 523.1 | |
| Aminopeptidase | XPNPEPL | AE114s7 | AAAGCAGGAGAAGTGTATGGTCAGTT | 115 | T | 5FLANK | Non-CDS | 0 | NM_006 | |
| P-like | | | AAATAAACTATAATA | | | | | | 523.1 | |
| Aminopeptidase | XPNPEPL | AE114s6 | AGATGACAGGCTGGGCGCAGTGGCTC | 116 | T | 5FLANK | Non-CDS | 0 | NM_006 | |
| P-like | | | ACGCCTTTAATTCCA | | | | | | 523.1 | |
| Aminopeptidase | XPNPEPL | AE114s9 | AGTTTCTAGAAGGACAGTCACCCAAG | 117 | C | 5FLANK | Non-CDS | 0 | NM_006 | |
| P-like | | | TGTTAAGAGTGGTTA | | | | | | 523.1 | |
| Tissue kallikrein | KLK1 | AE115s13 | AGCTCTCAGAAGCCAGTTCAGAGGCT | 118 | G | Intron4 | Non-CDS | 0 | NM_002 | |
| | | | GAGTCCCCTCCCTCA | | | | | | 257.1 | |
| Tissue kallikrein | KLK1 | AE115s12 | GCTCTCAGAAGCCAGTTCAGAGGCTG | 119 | A | Intron4 | Non-CDS | 0 | NM_002 | |
| | | | AGTCCCCTCCCTCAG | | | | | | 257.1 | |
| Tissue kallikrein | KLK1 | AE115s11 | AAGTCTGTCACCTFCTGGACGTGGGCT | 120 | G | Exon4 | Silent | 0 | NM_002 | 603 |
| | | | TTTTTGCACTCATC | | | | | | 257.1 | |
| Tissue kallikrein | KLK1 | AE115s14 | CTGCTFCCCTGGCCCTTTCTCCCTTGG | 121 | C | Intron3 | Non-CDS | 0 | NM_002 | |
| | | | ACGCAGGAGTCCCC | | | | | | 257.1 | |
| Tissue kallikrein | KLK1 | AE115s1 | CTTTAGCATTACAAAATCCAGTGTCCC | 122 | G | 5FLANK | Non-CDS | 0 | NM_002 | |
| | | | TCCCAAACAATGCT | | | | | | 257.1 | |
| Tissue kallikrein | KLK1 | AE115s4 | AATGAGCCTCCCACTTCTTGCTCCCAG | 123 | C | 5FLANK | Non-CDS | 0 | NM_002 | |
| | | | TTATGGGTGCTCAA | | | | | | 257.1 | |
| Tissue kallikrein | KLK1 | AE115s3 | GGTCTCTGAAGATAATTAGGAACTAG | 124 | A | 5FLANK | Non-CDS | 0 | NM_002 | |
| | | | ATTCCTGCACCTCAA | | | | | | 257.1 | |
| Aminopeptidase | XPNPEP2 | AE116s1 | GCATTCCTCTCTGGGAAATCACCTTCC | 125 | A | 5FLANK | Non-CDS | 1 | NM_003 | |
| P (membrane | | | TCGAACCCAAAGAG | | | | | | 399.3 | |
| bound) | | | | | | | | | | |
| Aminopeptidase | XPNPEP2 | AE116s2 | TGGGAATCAGCTTGCTGGAGGGAAGG | 126 | G | 5FLANK | Non-CDS | 1 | NM_003 | |
| P (membrane | | | GGACCGAATTAAGGA | | | | | | 399.3 | |
| bound) | | | | | | | | | | |
| Aminopeptidase | XPNPEP2 | AE116s4 | ACCCTCTGTCTGCTCGAGCCCAGGAA | 127 | C | Exon1 | Non-CDS | 1 | NM_003 | 175 |
| P (membrane | | | AGGCCTGAAGGAAGA | | | | | | 399.3 | |
| bound) | | | | | | | | | | |
| Aminopeptidase | XPNPEP2 | AE116s3 | GAGGCCGGGGAAAGAGCCCTCCCTCT | 128 | C | Exon1 | Non-CDS | 1 | NM_003 | 214 |
| P (membrane | | | CTCCCTTGTCCCTCC | | | | | | 399.3 | |
| bound) | | | | | | | | | | |
| Aminopeptidase | XPNPEP2 | AE116s29 | GCTTCCTGGAAGAGGCACTTGGTGTG | 129 | G | Intron3 | Non-CDS | 1 | NM_003 | |
| P (membrane | | | CTTGGGCTTGTAGCT | | | | | | 399.3 | |
| bound) | | | | | | | | | | |
| Aminopeptidase | XPNPEP2 | AE116s30 | CTGCAAGTTTCTCTGTTCTGCCCCAGG | 130 | C | Intron4 | Non-CDS | 1 | NM_003 | |
| P (membrane | | | AACTGCAGTGGTGA | | | | | | 399.3 | |
| bound) | | | | | | | | | | |
| Aminopeptidase | XPNPEP2 | AE116s32 | TATAAAGAAATGGACCTTGGTAGAAG | 131 | T | Intron6 | Non-CDS | 1 | NM_003 | |
| P (membrane | | | GAGGGGCGGTGGGAC | | | | | | 399.3 | |
| bound) | | | | | | | | | | |
| Aminopeptidase | XPNPEP2 | AE116s33 | AGGGAACCAGGACTAACTTTGCCTGA | 132 | G | Intron6 | Non-CDS | 1 | NM_003 | |
| P (membrane | | | ATCACAATTTTTTCC | | | | | | 399.3 | |
| bound) | | | | | | | | | | |
| Aminopeptidase | XPNPEP2 | AE116s7 | GATACCCAAGGTGGGTTTGCCAGGCC | 133 | C | Intron10 | Non-CDS | 1 | NM_003 | |
| P (membrane | | | CCAGCCCAAGCCAGG | | | | | | 399.3 | |
| bound) | | | | | | | | | | |
| Aminopeptidase | XPNPEP2 | AE116s18 | CAGAGGCGCCAGCTACTAGAGGAGTT | 134 | G | Exon21 | Silent | 1 | NM_003 | 2172 |
| P (membrane | | | CGAGTGGCTTCAACA | | | | | | 399.3 | |
| bound) | | | | | | | | | | |
| Aminopeptidase | XPNPEP2 | AE116s28 | TCCAAGACCTATGGAGAAGGTCCCAG | 135 | T | Intron21 | Non-CDS | 1 | NM_003 | 2445 |
| P (membrane | | | GCCCCAGGAACACAG | | | | | | 399.3 | |
| bound) | | | | | | | | | | |
| Aminopeptidase | XPNPEP2 | AE116s21 | ATTGGCCGTTAGCCACCTGGGTCCAC | 136 | G | Intron21 | Non-CDS | 1 | NM_003 | 3378 |
| P (membrane | | | ATCCTGCTAAGACGT | | | | | | 399.3 | |
| bound) | | | | | | | | | | |
| Soluble | GUCY1A2 | AE117s11 | AAACCCATITGCTCTGATGGCCTTGAA | 137 | C | Exon6 | Silent | 1 | NM_000 | 2169 |
| guanylate | | | GATGATGGAACTTTC | | | | | | 855.1 | |
| cyclase 1, alpha- | | | | | | | | | | |
| 2 subunit | | | | | | | | | | |
| Soluble | GUCY1A2 | AE117s13 | GGCCTCCTCCTTCCTGTCTTGGCCTTT | 138 | G | Intron7 | Non-CDS | 1 | NM_000 | |
| guanylate | | | GTTTTGGGCTGCTA | | | | | | 855.1 | |
| cyclase 1, alpha- | | | | | | | | | | |
| 2 subunit | | | | | | | | | | |
| Soluble | GUCY1A2 | AE117s7 | GAGACATCTTCAATTTCTTCTTTGTTG | 139 | T | Exon3 | Silent | 0 | NM_000 | 825 |
| guanylate | | | GAGTGGTCTGCATA | | | | | | 855.1 | |
| cyclase 1, alpha- | | | | | | | | | | |
| 2 subunit | | | | | | | | | | |
| Soluble | GUCY1A2 | AE117s6 | ACATCTTCAATTTCTTCTTTGTTGGAG | 140 | G | Exon3 | Silent | 0 | NM_000 | 822 |
| guanylate | | | TGGTCTGCATAGGA | | | | | | 855.1 | |
| cyclase 1, alpha- | | | | | | | | | | |
| 2 subunit | | | | | | | | | | |
| Soluble | GUCY1A2 | AE117s10 | AAGAAGAAAACAAAATTAGCATAAG | 141 | A | Intron2 | Non-CDS | 0 | NM_000 | |
| guanylate | | | GAGAAATTTTTGAAAG | | | | | | 855.1 | |
| cyclase 1, alpha- | | | | | | | | | | |
| 2 subunit | | | | | | | | | | |
| Soluble | GUCY1A2 | AE117s9 | TTTTTGAAAGCATCTGCGAATGCAACT | 142 | T | Intron2 | Non-CDS | 0 | NM_000 | |
| guanylate | | | CATATACACACGCA | | | | | | 855.1 | |
| cyclase 1, alpha- | | | | | | | | | | |
| 2 subunit | | | | | | | | | | |
| Soluble | GUCY1A2 | AE117s14 | TCCCTGGCCTACTTGATTCATGTGCTC | 143 | T | Intron7 | Non-CDS | 1 | NM_000 | |
| guanylate | | | TGTATTTTCTTCTTT | | | | | | 855.1 | |
| cyclase 1, alpha- | | | | | | | | | | |
| 2 subunit | | | | | | | | | | |
| Soluble | GUCY1A2 | AE117s15 | TTTCAGAACTAACCAATAAATAGATT | 144 | T | Exon8 | Non-CDS | 1 | NM_000 | 2882 |
| guanylate | | | CCATGTTTTCTTGTT | | | | | | 855.1 | |
| cyclase 1, alpha- | | | | | | | | | | |
| 2 subunit | | | | | | | | | | |
| Soluble | GUCY1A2 | AE117s1 | CAAAGAGCCCTGAGGATGAGGGACGT | 145 | G | 5FLANK | Non-CDS | 1 | NM_000 | |
| guanylate | | | GGGATCATTCCCCGG | | | | | | 855.1 | |
| cyclase 1, alpha- | | | | | | | | | | |
| 2 subunit | | | | | | | | | | |
| Soluble | GUCY1A2 | AE117s2 | ATCATTCCCCGGTTTCCCCCGGGCCAG | 146 | G | 5FLANK | Non-CDS | 1 | NM_000 | |
| guanylate | | | GTGCTAGGTCGTTT | | | | | | 855.1 | |
| cyclase 1, alpha- | | | | | | | | | | |
| 2 subunit | | | | | | | | | | |
| Soluble | GUCY1A2 | AE117s3 | AGGCGGCTATAGGTGGCAAGCGGAGC | 147 | C | 5FLANK | Non-CDS | 1 | NM_000 | |
| guanylate | | | TGTCCAGCACTAGTG | | | | | | 855.1 | |
| cyclase 1, alpha- | | | | | | | | | | |
| 2 subunit | | | | | | | | | | |

TABLE II

| | | | | REFSEQ_FLANK_ALT | | | | | | |
|---|---|---|---|---|---|---|---|---|---|---|
| | | | | SEQ | | | | | | |
| GENE_DESCR. | HGNC_ID | SNP_ID | REFSEQ_FLANK_ALT | ID NO: | ALT_NT | EXON | MUTATION_TYPE | REV_COMP | CDNA_SEQ_ID | CDNA _SEQ_POS |
| C1,S | C1S | AE111s1 | TATTGTTTTTTGTTTGTTTGCTTTCAAG | 148 | C | 5FLANK | Non-CDS | 1 | NM_00 | |
| subcomponent | | | TTTGGGACTAAAA | | | | | | 1734.1 | |
| C1,S | C1S | AE111s2 | CCTGCAGAGGGAGCGTCAAGACCCTG | 149 | A | Exon1 | Non-CDS | 1 | NM_00 | 32 |
| subcomponent | | | TGCTGCTGTCCCTGG | | | | | | 1734.1 | |
| C1,S | C1S | AE111s13 | GGCGGGAGGATTGCTTGAGCTCAGGA | 150 | T | Intron2 | Non-CDS | 1 | NM_00 | |
| subcomponent | | | CTTTGAGACCAGCCT | | | | | | 1734.1 | |
| C1,S | C1S | AE111s15 | GCACAAAAAGAATAGAGATGAAAGA | 151 | A | Intron3 | Non-CDS | 1 | NM_00 | |
| subcomponent | | | CTAGGGCTAAGGTAGC | | | | | | 1734.1 | |
| C1,S | C1S | AE111s17 | AGACTTTTCCAATGAAGAGCATTTTAC | 152 | A | Exon4 | Missense | 1 | NM_00 | 558 |
| subcomponent | | | GGGGTTTGCTGCAT | | | | | | 1734.1 | |
| C1,S | C1S | AE111s19 | TCTTACCCTTATGGTTTTGGGTTTAAC | 153 | G | Intron4 | Non-CDS | 1 | NM_00 | |
| subcomponent | | | CTCATTCTCCCTTC | | | | | | 1734.1 | |
| C1,S | C1S | AE111s18 | GTCCCTTGTAGCCACTTCTGTAACAAT | 154 | T | Exon5 | Silent | 1 | NM_00 | 643 |
| subcomponent | | | TTCATTGGTGGTTA | | | | | | 1734.1 | |
| C1,S | C1S | AE111s20 | GGATITCCCTGGGCCTCTAAAAATTGA | 155 | A | Exon7 | Missense | 1 | NM_00 | 982 |
| subcomponent | | | AACCAAGAGTAATGC | | | | | | 1734.1 | |
| C1,S | C1S | AE111s21 | CAATTCTGTTTGGGAGCCTGTGAAGG | 156 | T | Exon8 | Missense | 1 | NM_00 | 1122 |
| subcomponent | | | CAAAATATGTCTTTA | | | | | | 1734.1 | |
| C1,S | C1S | AE111s22 | AGCCTGCGAAGGCAAAATATATCTTT | 157 | A | Exon8 | Missense | 1 | NM_00 | 1136 |
| subcomponent | | | AGAGATGTGGTGCAG | | | | | | 1734.1 | |
| C1,S | C1S | AE111s5 | CCACAGAGAGGCTGGTGTGGAGAGGT | 158 | A | Intron9 | Non-CDS | 1 | NM_00 | |
| subcomponent | | | TCATCCCAGGTGTGC | | | | | | 1734.1 | |
| C1,S | C1S | AE111s6 | TTGGGGAGAGATGATAGCCACGGGTG | 159 | C | Intron1 | Non-CDS | 1 | NM_00 | |
| subcomponent | | | ACTGGGAGTCACCTT | | | | | | 1734.1 | |
| C1,S | C1S | AE111s8 | ACCTCTTCCGACTACAACCTTATGGAT | 160 | T | Exon12 | Silent | 1 | NM_00 | 1879 |
| subcomponent | | | GGGGACCTGGGACT | | | | | | 1734.1 | |
| alanyl | ANPEP | AE112s57 | TCCCAGACTCCACGGTGCTCTGCATGC | 161 | T | 3FLANK | Non-CDS | 0 | NM_00 | |
| aminopeptidase | | | ACCCAGCTTCCCCC | | | | | | 1150.1 | |
| alanyl | ANPEP | AE112s55 | GGCCCTGGAGCTGGGCTTCCTTGAGA | 162 | T | Exon20 | Non-CDS | 0 | NM_00 | 3296 |
| aminopeptidase | | | TCAGCCCCAGGGCAC | | | | | | 1150.1 | |
| alanyl | ANPEP | AE112s56 | CAGCCTGGGTCATCAGGAACCAGACT | 163 | C | Exon20 | Non-CDS | 0 | NM_00 | 3185 |
| aminopeptidase | | | GGCTCACAGGCAGAG | | | | | | 1150.1 | |
| alanyl | ANPEP | AE112s58 | ATGGAGGCCCTGCACCAGCCACTGGG | 164 | A | Exon20 | Non-CDS | 0 | NM_00 | 3084 |
| aminopeptidase | | | ATGGACACATGTGGG | | | | | | 1150.1 | |
| alanyl | ANPEP | AE112s44 | CTGGCTACTCGGCTTCCTCTATAAATG | 165 | A | Intron19 | Non-CDS | 0 | NM_00 | |
| aminopeptidase | | | AGGAAGGCCTGGCG | | | | | | 1150.1 | |
| alanyl | ANPEP | AE112s46 | TGTGCTCCCTCAGGGCCACGCGGGAG | 166 | C | Intron19 | Non-CDS | 0 | NM_00 | |
| aminopeptidase | | | AAGAATGGAGTGCCC | | | | | | 1150.1 | |
| alanyl | ANPEP | AE112s42 | AATGGAGTGCCCCTTTGGCCGGGGAA | 167 | G | Intron19 | Non-CDS | 0 | NM_00 | |
| aminopeptidase | | | GGAAGTAGGCAGAGC | | | | | | 1150.1 | |
| alanyl | ANPEP | AE112s43 | CCCCTTTGGCCTGGGAAGGATGTAGG | 168 | T | Intron19 | Non-CDS | 0 | NM_00 | |
| aminopeptidase | | | CAGAGCCCAGGTCTG | | | | | | 1150.1 | |
| alanyl | ANPEP | AE112s41 | TCACACCTACTGGGTAGGGAACCATG | 169 | A | Intron18 | Non-CDS | 0 | NM_00 | |
| aminopeptidase | | | ATTTATAGAGGAGATG | | | | | | 1150.1 | |
| alanyl | ANPEP | AE112s40 | CCCCCCGGGGCCCCAGCCTCAGCGCT | 170 | A | Intron18 | Non-CDS | 0 | NM_00 | |
| aminopeptidase | | | CGCTGTCCCTGACAC | | | | | | 1150.1 | |
| alanyl | ANPEP | AE112s36 | TCCCAGACCAGACCTTGCCCGATGAC | 171 | G | Exon18 | Silent | 0 | NM_00 | 2733 |
| aminopeptidase | | | GTTGTTGGTAATGCT | | | | | | 1150.1 | |
| alanyl | ANPEP | AE112s38 | CGGATTAAGTCCGGGTTCAGTGTGTA | 172 | T | Exon18 | Silent | 0 | NM_00 | 2664 |
| aminopeptidase | | | GCTCAGGTACCTAAG | | | | | | 1150.1 | |
| alanyl | ANPEP | AE112s35 | CTAAGAGGGCCAGATGCTGTGTCAGC | 173 | G | Intron17 | Non-CDS | 0 | NM_00 | |
| aminopeptidase | | | TACTGCTAATTCAGG | | | | | | 1150.1 | |
| alanyl | ANPEP | AE112s29 | CTCTCGCAGTCCCACCCTGCCCCAAGA | 174 | C | Intron17 | Non-CDS | 0 | NM_00 | |
| aminopeptidase | | | CTCACCTGTTCAGG | | | | | | 1150.1 | |
| alanyl | ANPEP | AE112s33 | TTTCGGAACTGCTCCCAGGCAAAGTC | 175 | A | Exon17 | Silent | 0 | NM_00 | 2553 |
| aminopeptidase | | | CCACTCCTCCTCCCC | | | | | | 1150.1 | |
| alanyl | ANPEP | AE112s32 | CCTCCCCGCCCTGGGCGATAACGTTGC | 176 | A | Exon17 | Missense | 0 | NM_00 | 2519 |
| aminopeptidase | | | AGTAGACGGTGGAC | | | | | | 1150.1 | |
| alanyl | ANPEP | AE112s30 | GCGATAGCGTTGCAGTAGACAGTGGA | 177 | A | Exon17 | Silent | 0 | NM_00 | 2505 |
| aminopeptidase | | | CCGCAGGTTGGGGTG | | | | | | 1150.1 | |
| alanyl | ANPEP | AE112s26 | TAGTTCTGGGGAAAAGAAAACATGAA | 178 | C | Intron14 | Non-CDS | 0 | NM_00 | |
| aminopeptidase | | | TCTCATCAAAGACTC | | | | | | 1150.1 | |
| alanyl | ANPEP | AE112s22 | GTGGGGACCCAGGGAGGGACATCCAG | 179 | A | Intron13 | Non-CDS | 0 | NM_00 | |
| aminopeptidase | | | GGAGCACAGGAGCTC | | | | | | 1150.1 | |
| alanyl | ANPEP | AE112s23 | GAGCTCAGGGCACAGCACGTAGCATA | 180 | A | Intron13 | Non-CDS | 0 | NM_00 | |
| aminopeptidase | | | TGGGAAGGGCAGCAG | | | | | | 1150.1 | |
| alanyl | ANPEP | AE112s21 | GAAGTCACGAGCTTCTGCAGTTGAGC | 181 | T | Intron13 | Non-CDS | 0 | NM_00 | |
| aminopeptidase | | | CAGGCAGCGGAGCAC | | | | | | 1150.1 | |
| alanyl | ANPEP | AE112s17 | TGTTGAATGGAATGGCCCATGACAGC | 182 | G | Intron10 | Non-CDS | 0 | NM_00 | |
| aminopeptidase | | | CCTCAGAACATGGGC | | | | | | 1150.1 | |
| alanyl | ANPEP | AE112s18 | GCTGGATTACCTCTTACATCCATCAGC | 183 | C | Exon11 | Missense | 0 | NM_00 | 1929 |
| aminopeptidase | | | CAGTAGTCCTGCTG | | | | | | 1150.1 | |
| alanyl | ANPEP | AE112s19 | CTGGATTACCTCTTACATCTTTCAGCC | 184 | T | Exon11 | Missense | 0 | NM_00 | 1928 |
| aminopeptidase | | | AGTAGTCCTGCTGC | | | | | | 1150.1 | |
| alanyl | ANPEP | AE112s71 | CCCTCCAGGCCAAGTCCCCATCTCCTT | 185 | T | Intron7 | Non-CDS | 0 | NM_00 | |
| aminopeptidase | | | CCCCGTGCCCCACG | | | | | | 1150.1 | |
| alanyl | ANPEP | AE112s72 | ACCTCCTTCCCCGTGCCCCATGAGGAG | 186 | T | Intron7 | Non-CDS | 0 | NM_00 | |
| aminopeptidase | | | CGGGCTGCACCTTG | | | | | | 1150.1 | |
| alanyl | ANPEP | AE112s69 | CATGCCCCCCGCACCAGACCTCTGGG | 187 | T | Intron6 | Non-CDS | 0 | NM_00 | |
| aminopeptidase | | | CAGCTGGCTTACCAA | | | | | | 1150.1 | |
| alanyl | ANPEP | AE112s68 | CTGGAGGAGGACAGGGGGTCAAACA | 188 | A | Exon5 | Silent | 0 | NM_00 | 1227 |
| aminopeptidase | | | GCAGGGAGTTCTCCCG | | | | | | 1150.1 | |
| alanyl | ANPEP | AE112s64 | TCTGGTCTGGGGAGGCGATGTCATTG | 189 | T | Intron4 | Non-CDS | 0 | NM_00 | |
| aminopeptidase | | | GCAGGATGAACTCCG | | | | | | 1150.1 | |
| alanyl | ANPEP | AE112s63 | AAGAAGTTAAGGATGGGGCCTGTCAC | 190 | T | Exon4 | Silent | 0 | NM_00 | 1083 |
| aminopeptidase | | | GTTCAGGGCATAATC | | | | | | 1150.1 | |
| alanyl | ANPEP | AE112s62 | AGGATGGGGCCCGTCACGTTAAGGGC | 191 | A | Exon4 | Silent | 0 | NM_00 | 1074 |
| aminopeptidase | | | ATAATCGCCGTGGCC | | | | | | 1150.1 | |
| alanyl | ANPEP | AE112s61 | GGGCATAATCGCCGTGGCCCACCGCA | 192 | A | Exon4 | Missense | 0 | NM_00 | 1052 |
| aminopeptidase | | | ATGGCACTGGGCCGG | | | | | | 1150.1 | |
| alanyl | ANPEP | AE112s65 | CAGGTGGGCTGGGTCCCAGGACCCAG | 193 | A | Intron3 | Non-CDS | 0 | NM_00 | |
| aminopeptidase | | | TGGGCTGGGGGGATC | | | | | | 1150.1 | |
| alanyl | ANPEP | AE112s53 | CCTGGCCCTGTGGCCGCAGGAAGGGC | 194 | A | Intron2 | Non-CDS | 0 | NM_00 | |
| aminopeptidase | | | CCACTCACCTTTGGG | | | | | | 1150.1 | |
| alanyl | ANPEP | AE112s52 | TCTGCAGCCTGCATCTGTGTTGTGGCC | 195 | T | Exon2 | Silent | 0 | NM_00 | 747 |
| aminopeptidase | | | ACCACCCTGCCCCA | | | | | | 1150.1 | |
| alanyl | ANPEP | AE112s13 | AGCACCTCGGATCCACCCCAGCGGGC | 196 | G | Intron1 | Non-CDS | 0 | NM_00 | |
| aminopeptidase | | | AGCCCAGCCGGAACT | | | | | | 1150.1 | |
| alanyl | ANPEP | AE112s15 | TTGCTGTGGATGATGATGACATCAGT | 197 | A | Exon1 | Silent | 0 | NM_00 | 474 |
| aminopeptidase | | | GGCCTCCTTGCAGGT | | | | | | 1150.1 | |
| alanyl | ANPEP | AE112s1 | GGCTCCAACAGGCGAAGGTCGCTGGA | 198 | G | 5FLANK | Non-CDS | 0 | NM_00 | |
| aminopeptidase | | | CTGGGCAGGGGCACG | | | | | | 1150.1 | |
| alanyl | ANPEP | AE112s5 | AGTTCCTCCAGGTTCCCCTCACTGCCG | 199 | A | 5FLANK | Non-CDS | 0 | NM_00 | |
| aminopeptidase | | | CTTCTTGCCAAATA | | | | | | 1150.1 | |
| alanyl | ANPEP | AE112s4 | TAAAATGAAATGAGTTGTTTCGCTTTT | 200 | C | 5FLANK | Non-CDS | 0 | NM_00 | |
| aminopeptidase | | | TTTGCTGAAGGCTT | | | | | | 1150.1 | |
| alanyl | ANPEP | AE112s3 | CAGGTTTGTTGAACAGATTTCGTGAG | 201 | C | 5FLANK | Non-CDS | 0 | NM_00 | |
| aminopeptidase | | | AAAACATATTAAACA | | | | | | 1150.1 | |
| alanyl | ANPEP | AE112s2 | GTGAGAAAACATATTAAACAGCAAAT | 202 | G | 5FLANK | Non-CDS | 0 | NM_00 | |
| aminopeptidase | | | AGTAGAATGATTAAA | | | | | | 1150.1 | |
| alanyl | ANPEP | AE112s9 | AGGCCGAGGTGGGTGGATCATGAGGT | 203 | T | 5FLANK | Non-CDS | 0 | NM_00 | |
| aminopeptidase | | | TAGGAGTTCAAGACC | | | | | | 1150.1 | |
| alanyl | ANPEP | AE112s6 | AGTTGGGCGTGGTGGCGGGCATCTGT | 204 | A | 5FLANK | Non-CDS | 0 | NM_00 | |
| aminopeptidase | | | AATCCCAGCTACTTG | | | | | | 1150.1 | |
| alanyl | ANPEP | AE112s7 | GGCAGAGAATTGCTTGAATCTGGGAG | 205 | T | 5FLANK | Non-CDS | 0 | NM_00 | |
| aminopeptidase | | | GCAGAGATTGCAGTG | | | | | | 1150.1 | |
| alanyl | ANPEP | AE112s11 | ATTGCAGTGAGCTGAGATCGAACCAC | 206 | A | 5FLANK | Non-CDS | 0 | NM_00 | |
| aminopeptidase | | | TGCACTCCAGCCTGG | | | | | | 1150.1 | |
| alanyl | ANPEP | AE112s12 | TTGCAGTGAGCTGAGATCGCCCCACT | 207 | C | 5FLANK | Non-CDS | 0 | NM_00 | |
| aminopeptidase | | | GCACTCCAGCCTGGG | | | | | | 1150.1 | |
| meprin A, beta | MEP1B | AE113s4 | GTGGTGTGATCTCGGCTCACTGCAACC | 208 | T | 5FLANK | Non-CDS | 1 | NM_00 | |
| | | | TCTGCCTCCCAGGT | | | | | | 5925.1 | |
| meprin A, beta | MEP1B | AE113s3 | AGCTGGGATTACAGACATGCCCCACC | 209 | C | 5FLANK | Non-CDS | 1 | NM_00 | |
| | | | ACACCTGGCTAATTT | | | | | | 5925.1 | |
| meprin A, beta | MEP1B | AE113s2 | TCTAGTAGAGATGGGGTTTCCCTGTGT | 210 | C | 5FLANK | Non-CDS | 1 | NM_00 | |
| | | | TGGCCAGGTTGGGC | | | | | | 5925.1 | |
| meprin A, beta | MEP1B | AE113s1 | GTTGGCCAGGTTGGGCTTGACCTCCCG | 211 | C | 5FLANK | Non-CDS | 1 | NM_00 | |
| | | | ACCTCAGGTGGTCC | | | | | | 5925.1 | |
| meprin A, beta | MEP1B | AE113s5 | ATAGGCACTCAATAATTTTTAAAT | 212 | G | 5FLANK | Non-CDS | 1 | NM_00 | |
| | | | GAGTGAATGATAAA | | | | | | 5925.1 | |
| meprin A, beta | MEP1B | AE113s34 | TTGGGACTGGATCTTTTTGAAGGTGAC | 213 | A | Exon3 | Silent | 1 | NM_00 | 196 |
| | | | ATCAGACTTGATAG | | | | | | 5925.1 | |
| meprin A, beta | MEP1B | AE113s37 | TTCAAAGGGTGGGCTTATATGGTCTTT | 214 | G | Intron5 | Non-CDS | 1 | NM_00 | |
| | | | AAGGTTTTGTTTGG | | | | | | 5925.1 | |
| meprin A, beta | MEP1B | AE113s38 | GCTGGAGAAACAAACTATATGTCAGT | 215 | G | Exon6 | Missense | 1 | NM_00 | 394 |
| | | | GTTCAAGGGCAGTGG | | | | | | 5925.1 | |
| meprin A, beta | MEP1B | AE113s39 | CTGCCCTGAGACCTCAGATTTTAACAT | 216 | T | Intron6 | Non-CDS | 1 | NM_00 | |
| | | | TCTCTTCCCCCTTT | | | | | | 5925.1 | |
| meprin A, beta | MEP1B | AE113s41 | ACATTTCTCTTTTTTTCCTACGTTTTTA | 217 | C | Intron7 | Non-CDS | 1 | NM_00 | |
| | | | GTTTAAGGACTGAA | | | | | | 5925.1 | |
| meprin A, beta | MEP1B | AE113s40 | TTTAGTTAAGGACTGAATTTTTAAGCA | 218 | T | Intron7 | Non-CDS | 1 | NM_00 | |
| | | | TGTGTCCTCTCTTG | | | | | | 5925.1 | |
| meprin A, beta | MEP1B | AE113s43 | TCCTTGAGTTTTATGGACTCATGCAGT | 219 | A | Exon9 | Silent | 1 | NM_00 | 838 |
| | | | TTTGAACTGGAAAA | | | | | | 5925.1 | |
| meprin A, beta | MEP1B | AE113s42 | TAATGATTATTCATTAGGTTGCTACTG | 220 | G | Intron9 | Non-CDS | 1 | NM_00 | |
| | | | AGAAGTAGGCCTTG | | | | | | 5925.1 | |
| meprin A, beta | MEP1B | AE113s7 | CTCTCAATTCTGCTTTTCTTCAACAGG | 221 | C | Intron9 | Non-CDS | 1 | NM_00 | |
| | | | TTCTGGTTTCTTCA | | | | | | 5925.1 | |
| meprin A, beta | MEP1B | AE113s6 | TAAATGTGGGGGCCACAGCAATGCTG | 222 | A | Exon10 | Missense | 1 | NM_00 | 1022 |
| | | | GAAAGTAGAACGCTG | | | | | | 5925.1 | |
| meprin A, beta | MEP1B | AE113s9 | TGTTTGAAGGACGCAAAGGCACTGGT | 223 | A | Exon10 | Missense | 1 | NM_00 | 1268 |
| | | | GCATCACTGGGTGGT | | | | | | 5925.1 | |
| meprin A, beta | MEP1B | AE113s8 | ATTGATGACATCAATCTTTCAGAAAC | 224 | A | Exon10 | Silent | 1 | NM_00 | 1315 |
| | | | ACGGTGCCCTCATCA | | | | | | 5925.1 | |
| meprin A, beta | MEP1B | AE113s18 | AACCAGTGCCTTTATAACCCCCGAAA | 225 | C | Exon12 | Missense | 1 | NM_00 | 1740 |
| | | | GGCTGAAAAGCAGAG | | | | | | 5925.1 | |
| meprin A, beta | MEP1B | AE113s14 | ACCAGTGCCTTTATAACCCATGAAAG | 226 | T | Exon12 | Silent | 1 | NM_00 | 1741 |
| | | | GCTGAAAAGCAGAGA | | | | | | 5925.1 | |
| meprin A, beta | MEP1B | AE113s16 | TTTATATAAACTAGTTTTTTATTGTTGT | 227 | A | Intron12 | Non-CDS | 1 | NM_00 | |
| | | | GGCCACTGATTAT | | | | | | 5925.1 | |
| meprin A, beta | MEP1B | AE113s21 | TTCCACTTTTAGATGTATTAGATAGAG | 228 | G | Intron12 | Non-CDS | 1 | NM_00 | |
| | | | ATGTGGGGGGAATG | | | | | | 5925.1 | |
| mepnn A, beta | MEP1B | AE113s22 | ATCAAATGATTGTTAAACAACAGCTG | 229 | C | Intron12 | Non-CDS | 1 | NM_00 | |
| | | | ATTTCCATCCACACT | | | | | | 5925.1 | |
| meprin A, beta | MEP1B | AE113s24 | AAGAGAGGCTCCACCCGAGATACCAT | 230 | T | Exon14 | Silent | 1 | NM_00 | 1999 |
| | | | AGTCATTGCTGTTTC | | | | | | 5925.1 | |
| meprin A, beta | MEP1B | AE113s25 | AAATCGACCAAATTTGACTCTGCAAA | 231 | T | Exon14 | Missense | 1 | NM_00 | 2130 |
| | | | ATGTAAGTTGAGGCT | | | | | | 5925.1 | |
| meprin A, beta | MEP1B | AE113s26 | GGCTGATGTTTGATTATTCACAACCTA | 232 | C | Intron14 | Non-CDS | 1 | NM_00 | |
| | | | TTGGTGAAATCTTA | | | | | | 5925.1 | |
| meprin A, beta | MEP1B | AE113s31 | CTTCATTTAAAGACCAGATCGTTTTAT | 233 | 0 | Intron14 | Non-CDS | 1 | NM_00 | |
| | | | TATGATTCATTTAA | | | | | | 5925.1 | |
| meprin A, beta | MEP1B | AE113s30 | AAAGTGGATATTTTTCTGTACATAGCT | 234 | C | Exon15 | Non-CDS | 1 | NM_00 | 2265 |
| | | | GGAAATATTATAAA | | | | | | 5925.1 | |
| Aminopeptidase | XPNPEPL | AE114s30 | TTCCCAATCACATCCCTGCACGTCAGG | 235 | C | Exon19 | Silent | 0 | NM_00 | 1773 |
| P-like | | | TGGTAATTGTTGAG | | | | | | 6523.1 | |
| Aminopeptidase | XPNPEPL | AE114s31 | CACAAGCTGAGAGAAAAGGTATGCAA | 236 | A | Intron18 | Non-CDS | 0 | NM_00 | |
| P-like | | | CACCCTTTCTCCTTA | | | | | | 6523.1 | |
| Aminopeptidase | XPNPEPL | AE114s29 | ATITAAAAAATACAAAACAACTAATAG | 237 | T | Intron16 | Non-CDS | 0 | NM_00 | |
| P-like | | | CTTTCAAAAAGGCTC | | | | | | 6523.1 | |
| Aminopeptidase | XPNPEPL | AE114s28 | AAGAAGGTGACCTGAAAGACGTAAAG | 238 | G | Intron15 | Non-CDS | 0 | NM_00 | |
| P-like | | | AGCCACTTAATTGTA | | | | | | 6523.1 | |
| Aminopeptidase | XPNPEPL | AE114s26 | TGCAGAATGGGGACAGATGTGTAATC | 239 | G | Intron14 | Non-CDS | 0 | NM_00 | |
| P-like | | | CTGTAGTTTTCTGGT | | | | | | 6523.1 | |
| Aminopeptidase | XPNPEPL | AE114s25 | AGGAGCAATTTTACTACCCCCCATGTA | 240 | C | Intron14 | Non-CDS | 0 | NM_00 | |
| P-like | | | ATTTTTACTTCTTT | | | | | | 6523.1 | |
| Aminopeptidase | XPNPEPL | AE114s24 | GAGCAATTTTACTACCCCACGTGTAAT | 241 | G | Intron14 | Non-CDS | 0 | NM_00 | |
| P-like | | | TTTTACTTCTTTAT | | | | | | 6523.1 | |
| Aminopeptidase | XPNPEPL | AE114s23 | TATCAAACAGTCTTTTGTGAAAACAG | 242 | A | Intron13 | Non-CDS | 0 | NM_00 | |
| P-like | | | AGTCACAACTGGGCT | | | | | | 6523.1 | |
| Aminopeptidase | XPNPEPL | AE114s22 | ACCAGCTGAGTGCTCTCAGCCGGAGC | 243 | C | Intron13 | Non-CDS | 0 | NM_00 | |
| P-like | | | TCAGCTGTCCACCTT | | | | | | 6523.1 | |
| Aminopeptidase | XPNPEPL | AE114s18 | AGCAGCAAGGCCAGGCAGCACGCTCC | 244 | C | Intron12 | Non-CDS | 0 | NM_00 | |
| P-like | | | TCCGCATGCCTTACG | | | | | | 6523.1 | |
| Aminopeptidase | XPNPEPL | AE114s14 | CTGCCTGTTGCCTGTAACAGCAAGAC | 245 | C | Intron11 | Non-CDS | 0 | NM_00 | |
| P-like | | | AGAAAGCAGAGTGGC | | | | | | 6523.1 | |
| Aminopeptidase | XPNPEPL | AE114s13 | CACGAGGCTCCTGGGTCCCCTCAAAT | 246 | T | Intron11 | Non-CDS | 0 | NM_00 | |
| P-like | | | GGATCCTTACCTGCG | | | | | | 6523.1 | |
| Aminopeptidase | XPNPEPL | AE114s16 | AAAGCTCACTTTTTCCTCAACTGCTTC | 247 | C | Intron10 | Non-CDS | 0 | NM_00 | |
| P-like | | | ATCCCAACTGACCA | | | | | | 6523.1 | |
| Aminopeptidase | XPNPEPL | AE114s12 | GCATCCACTCTATTATGGAATTTTTCT | 248 | T | Intron10 | Non-CDS | 0 | NM_00 | |
| P-like | | | TCATTCTTGTTACT | | | | | | 6523.1 | |
| Aminopeptidase | XPNPEPL | AE114s45 | GGCTGAGGCTCAGAGAGATTGTAACC | 249 | G | Intron9 | Non-CDS | 0 | NM_00 | |
| P-like | | | TTGTGCCAGGCTCAA | | | | | | 6523.1 | |
| Aminopeptidase | XPNPEPL | AE114s44 | CGGTGGTCCTAGAGGCAAAGAGCAGT | 250 | A | Intron8 | Non-CDS | 0 | NM_00 | |
| P-like | | | AGGGTGGGAAATCAA | | | | | | 6523.1 | |
| Aminopeptidase | XPNPEPL | AE114s43 | GAGCCTGCAGATGGAGGAGAAGTGGG | 251 | A | Intron7 | Non-CDS | 0 | NM_00 | |
| P-like | | | TGGCAGAAAAAGGAG | | | | | | 6523.1 | |
| Aminopeptidase | XPNPEPL | AE114s42 | AATATCAGGCAGTGATGGGTAGGCCA | 252 | A | Intron7 | Non-CDS | 0 | NM_00 | |
| P-like | | | GGACCAGAAAGGGCA | | | | | | 6523.1 | |
| Aminopeptidase | XPNPEPL | AE114s41 | CAGCCCTGGTCCCAGAAAGCGGTAGG | 253 | G | Intron5 | Non-CDS | 0 | NM_00 | |
| P-like | | | AACCCAGTGAAAGAA | | | | | | 6523.1 | |
| Aminopeptidase | XPNPEPL | AE114s35 | TCGACTTGGATTCGGAGGACAAGGCT | 254 | A | Intron5 | Non-CDS | 0 | NM_00 | |
| P-like | | | GGAGACAATAGCAAG | | | | | | 6523.1 | |
| Aminopeptidase | XPNPEPL | AE114s34 | CTTGGATTCGGAGGACGAGGTTGGAG | 255 | T | Intron5 | Non-CDS | 0 | NM_00 | |
| P-like | | | ACAATAGCAAGAGGT | | | | | | 6523.1 | |
| Aminopeptidase | XPNPEPL | AE114s36 | ACGCCCATTGCAGCAGGGCACGGAGC | 256 | C | Intron4 | Non-CDS | 0 | NM_00 | |
| P-like | | | AGGGCCTTGAAAGTC | | | | | | 6523.1 | |
| Aminopeptidase | XPNPEPL | AE114s38 | GCCCATTGCAGCAGGGCAGGAAGCAG | 257 | A | Intron4 | Non-CDS | 0 | NM_00 | |
| P-like | | | GGCCTTGAAAGTCCA | | | | | | 6523.1 | |
| Aminopeptidase | XPNPEPL | AB114533 | GCCTGGAGAAAGTAGCGCCCATCAGT | 258 | A | Exon3 | Silent | 0 | NM_00 | 225 |
| P-like | | | CCACATGGCTGCATG | | | | | | 6523.1 | |
| Aminopeptidase | XPNPEPL | AE114s32 | GACTGTTCTGCCTGCTTAGCTTTGTGC | 259 | T | Intron1 | Non-CDS | 0 | NM_00 | |
| P-like | | | TAGGCTCAGCGGGG | | | | | | 6523.1 | |
| Aminopeptidase | XPNPEPL | AE114s5 | ATTTCAAAACCAGCCTGCCCTCAGCA | 260 | T | 5FLANK | Non-CDS | 0 | NM_00 | |
| P-like | | | ATTCAACGTCCAGTT | | | | | | 6523.1 | |
| Arninopeptidase | XPNPEPL | AE114s3 | TAGGTGTTTATCTTTGTTTACAGTAGA | 261 | C | 5FLANK | Non-CDS | 0 | NM_00 | |
| P-like | | | AAAAAGCAGGAGAA | | | | | | 6523.1 | |
| Arninopeptidase | XPNPEPL | AE114s7 | AAAGCAGGAGAAGTGTATGGGCAGTT | 262 | G | 5FLANK | Non-CDS | 0 | NM_00 | |
| P-like | | | AAATAAACTATAATA | | | | | | 6523.1 | |
| Aminopeptidase | XPNPEPL | AE114s6 | AGATGACAGGCTGGGCGCAGCGGCTC | 263 | C | 5FLANK | Non-CDS | 0 | NM_00 | |
| P-like | | | ACGCCTTTAATTCCA | | | | | | 6523.1 | |
| Aminopeptidase | XPNPEPL | AE114s9 | AGTTTCTAGAAGGACAGTCATCCAAG | 264 | T | 5FLANK | Non-CDS | 0 | NM_00 | |
| P-like | | | TGTTAAGAGTGGTTA | | | | | | 6523.1 | |
| Tissue kallikrein | KLK1 | AE115s13 | AGCTCTCAGAAGCCAGTTCACAGGCT | 265 | C | Intron4 | Non-CDS | 0 | NM_00 | |
| | | | GAGTCCCCTCCCTCA | | | | | | 2257.1 | |
| Tissue kallikrein | KLK1 | AE115s12 | GCTCTCAGAAGCCAGTTCAGGGGCTG | 266 | C | Intron4 | Non-CDS | 0 | NM_00 | |
| | | | AGTCCCCTCCCTCAG | | | | | | 2257.1 | |
| Tissue kallikrein | KLK1 | AE115s11 | AAGTCTGTCACCTTCTGGACATGGGCT | 267 | A | Exon4 | Silent | 0 | NM_00 | 603 |
| | | | TTTTTGCACTCATC | | | | | | 2257.1 | |
| Tissue kallikrein | KLK1 | AE115s14 | CTGCTTCCCTGGCCCTTTCTACCTTGG | 268 | A | Intron3 | Non-CDS | 0 | NM_00 | |
| | | | ACGCAGGAGTCCCC | | | | | | 2257.1 | |
| Tissue kallikrein | KLK1 | AE115s1 | CTTTAGCATTACAAAATCCAATGTCCC | 269 | A | 5FLANK | Non-CDS | 0 | NM_00 | |
| | | | TCCCAAACAATGCT | | | | | | 2257.1 | |
| Tissue kallikrein | KLK1 | AE115s4 | AATGAGCCTCCCACTTCTTGTTCCCAG | 270 | T | 5FLANK | Non-CDS | 0 | NM_00 | |
| | | | TTATGGGTGCTCAA | | | | | | 2257.1 | |
| Tissue kallikrein | KLK1 | AE115s3 | GGTCTCTGAAGATAATTAGGGACTAG | 271 | G | 5FLANK | Non-CDS | 0 | NM_00 | |
| | | | ATTCCTGCACCTCAA | | | | | | 2257.1 | |
| Aminopeptidase | XPNPEP2 | AE116s1 | GCATTCCTCTCTGGGAAATCGCCTTCC | 272 | G | 5FLANK | Non-CDS | 1 | NM_00 | |
| P (membrane | | | TCGAACCCAAAGAG | | | | | | 3399.3 | |
| bound) | | | | | | | | | | |
| Aminopeptidase | XPNPEP2 | AE116s2 | TGGGAATCAGCTTGCTGGAGAGAAGG | 273 | A | 5FLANK | Non-CDS | 1 | NM_00 | |
| P (membrane | | | GGACCGAATTAAGGA | | | | | | 3399.3 | |
| bound) | | | | | | | | | | |
| Aminopeptidase | XPNPEP2 | AE116s4 | ACCCTCTGTCTGCTCGAGCCTAGGAA | 274 | T | Exon1 | Non-CDS | 1 | NM_00 | 175 |
| P (membrane | | | AGGCCTGAAGGAAGA | | | | | | 3399.3 | |
| bound) | | | | | | | | | | |
| Aminopeptidase | XPNPEP2 | AE116s3 | GAGGCCGGGGAAAGAGCCCTGCCTCT | 275 | G | Exon1 | Non-CDS | 1 | NM_00 | 214 |
| P (membrane | | | CTCCCTTGTCCCTCC | | | | | | 3399.3 | |
| bound) | | | | | | | | | | |
| Aminopeptidase | XPNPEP2 | AE116s29 | GCTTCCTGGAAGAGGCACTTAGTGTG | 276 | A | Intron3 | Non-CDS | 1 | NM_00 | |
| P (membrane | | | CTTGGGCTTGTAGCT | | | | | | 3399.3 | |
| bound) | | | | | | | | | | |
| Aminopeptidase | XPNPEP2 | AE116s30 | CTGCAAGTTTCTCTGTTCTGTCCCAGG | 277 | T | Intron4 | Non-CDS | 1 | NM_00 | |
| P (membrane | | | AACTGCAGTGGTGA | | | | | | 3399.3 | |
| bound) | | | | | | | | | | |
| Aminopeptidase | XPNPEP2 | AE116s32 | TATAAAGAAATGGACCTTGGGAGAAG | 278 | G | Intron6 | Non-CDS | 1 | NM_00 | |
| P (membrane | | | GAGGGGCGGTGGGAC | | | | | | 3399.3 | |
| bound) | | | | | | | | | | |
| Aminopeptidase | XPNPEP2 | AE116s33 | AGGGAACCAGGACTAACTTTACCTGA | 279 | A | Intron6 | Non-CDS | 1 | NM_00 | |
| P (membrane | | | ATCACAATTTTTTCC | | | | | | 3399.3 | |
| bound) | | | | | | | | | | |
| Aminopeptidase | XPNPEP2 | AE116s7 | GATACCCAAGGTGGGTTTGCTAGGCC | 280 | T | Intron10 | Non-CDS | 1 | NM_00 | |
| P (membrane | | | CCAGCCCAAGCCAGG | | | | | | 3399.3 | |
| bound) | | | | | | | | | | |
| Aminopeptidase | XPNPEP2 | AE116s18 | CAGAGGCGCCAGCTACTAGAAGAGTT | 281 | A | Exon21 | Silent | 1 | NM_00 | 2172 |
| P (membrane | | | CGAGTGGCTTCAACA | | | | | | 3399.3 | |
| bound) | | | | | | | | | | |
| Aminopeptidase | XPNPEP2 | AE116s28 | TCCAAGACCTATGGAGAAGGGCCCAG | 282 | G | Intron21 | Non-CDS | 1 | NM_00 | 2445 |
| P (membrane | | | GCCCCAGGAACACAG | | | | | | 3399.3 | |
| bound) | | | | | | | | | | |
| Aminopeptidase | XPNPEP2 | AE116s21 | ATTGGCCGTTAGCCACCTGGTTCCACA | 283 | T | Intron21 | Non-CDS | 1 | NM_00 | 3378 |
| P (membrane | | | TCCTGCTAAGACGT | | | | | | 3399.3 | |
| bound) | | | | | | | | | | |
| Soluble | GUCY1A2 | AE117s11 | AAACCCATTGCTCTGATGGCTTTGAAG | 284 | T | Exon6 | Silent | 1 | NM_00 | 2169 |
| guanylare | | | ATGATGGAACTTTC | | | | | | 0855.1 | |
| cyclase 1, alpha- | | | | | | | | | | |
| 2 subunit | | | | | | | | | | |
| Soluble | GUCY1A2 | AE117s13 | GGCCTCCTCCTTCCTGTCTTTGCCTTTG | 285 | T | Intron7 | Non-CDS | 1 | NM_00 | |
| guanylate | | | TTTTGGGCTGCTA | | | | | | 0855.1 | |
| cyclase 1, alpha- | | | | | | | | | | |
| 2 subunit | | | | | | | | | | |
| Soluble | GUCY1A2 | AE117s7 | GAGACATGTTCAATTTCTTCCTTGTTG | 286 | C | Exon3 | Silent | 0 | NM_00 | 825 |
| guanylate | | | GAGTGGTCTGCATA | | | | | | 0855.1 | |
| cyclase 1, alpha- | | | | | | | | | | |
| 2 subunit | | | | | | | | | | |
| Soluble | GUCY1A2 | AE117s6 | ACATCTTCAATTTCTTCTTTATTGGAG | 287 | A | Exon3 | Silent | 0 | NM_00 | 822 |
| guanylate | | | TGGTCTGCATAGGA | | | | | | 0855.1 | |
| cyclase 1, alpha- | | | | | | | | | | |
| 2 subunit | | | | | | | | | | |
| Soluble | GUCY1A2 | AE117s10 | AAGAAGAAAACAAAATTAGCTTAAGG | 288 | T | Intron2 | Non-CDS | 0 | NM_00 | |
| guanylate | | | AGAAATTTTTGAAAG | | | | | | 0855.1 | |
| cyclase 1, alpha- | | | | | | | | | | |
| 2 subunit | | | | | | | | | | |
| Soluble | GUCY1A2 | AF117s9 | TTTTTGAAAGCATCTGCGAACGCAACT | 289 | C | Intron2 | Non-CDS | 0 | NM_00 | |
| guanylate | | | CATATACACACGCA | | | | | | 0855.1 | |
| cyclase 1, alpha- | | | | | | | | | | |
| 2 subunit | | | | | | | | | | |
| Soluble | GUCY1A2 | AE117s14 | TCCCTGGCCTACTTGATTCACGTGCTC | 290 | C | Intron7 | Non-CDS | 1 | NM_00 | |
| guanylate | | | TGTATTTTCTTCTT | | | | | | 0855.1 | |
| cyclase 1, alpha- | | | | | | | | | | |
| 2 subunit | | | | | | | | | | |
| Soluble | GUCY1A2 | AE117s15 | TTTCAGAACTAACCAATAAACAGATT | 291 | C | Exon8 | Non-CDS | 1 | NM_00 | 2882 |
| guanylate | | | CCATGTTTTCTTGTT | | | | | | 0855.1 | |
| cyclase 1, alpha- | | | | | | | | | | |
| 2 subunit | | | | | | | | | | |
| Soluble | GUCY1A2 | AE117s1 | CAAAGAGCCCTGAGGATGAGAGACGT | 292 | A | 5FLANK | Non-CDS | 1 | NM_00 | |
| guanylate | | | GGGATCATTCCCCGG | | | | | | 0855.1 | |
| cyclase 1, alpha- | | | | | | | | | | |
| 2 subunit | | | | | | | | | | |
| Soluble | GUCY1A2 | AE117s2 | ATCATTCCCCGGTTTCCCCCCGGCCAG | 293 | C | 5FLANK | Non-CDS | 1 | NM_00 | |
| guanylate | | | GTGCTAGGTCGTTT | | | | | | 0855.1 | |
| cyclase 1, alpha- | | | | | | | | | | |
| 2 subunit | | | | | | | | | | |
| Soluble | GUCY1A2 | AE117s3 | AGGCGGCTATAGGTGGCAAGTGGAGC | 294 | T | 5FLANK | Non-CDS | 1 | NM_00 | |
| guanylate | | | TGTCCAGCACTAGTG | | | | | | 0855.1 | |
| cyclase 1, alpha- | | | | | | | | | | |
| 2 subunit | | | | | | | | | | |

TABLE III

| | | | | | | | | | | MUTA- | | | | PRO- | PRO- |
|---|---|---|---|---|---|---|---|---|---|---|---|---|---|---|---|
| GENE_DES- | | | CONTIG_{—} | | | | | ALT | | TION_{—} | REV | REF_{—} | ALT_{—} | TEIN_{—} | TEIN_{—} |
| CRIPTION | HGNC_ID | SNP_ID | NUM | CONTIG_POS | REF_SEQ_ID | REF_SEQ_POS | REF_AA | _AA | EXON | TYPE | COMP | CODON | CODON | ID | POS |
| C1,S | C1S | AE111s1 | AE111_X | 824 | AC00 | 14549 | | | 5FLANK | Non- | 1 | | | NP_00 | |
| subcomponent | | | O.f1:Conti | | 6512.1 | 3 | | | | CDS | | | | 1725.1 | |
| | | | g2 | | 2 | | | | | | | | | | |
| C1,S | C1S | AE111s2 | AE111_X | 1033 | AC00 | 14634 | | | Exon1 | Non- | 1 | | | NP_00 | |
| subcomponent | | | 0.f3:Conti | | 6512.1 | 9 | | | | CDS | | | | 1725.1 | |
| | | | g1 | | 2 | | | | | | | | | | |
| C1,S | C1S | AE111s1 | AE111_X | 314 | AC00 | 14762 | | | Intron2 | Non- | 1 | | | NP_00 | |
| subcomponent | | 3 | 2:Contig3 | | 6512.1 | 7 | | | | CDS | | | | 1725.1 | |
| | | | | | 2 | | | | | | | | | | |
| C1,S | C1S | AE111s1 | AE111_X | 346 | AC00 | 14828 | | | Intron3 | Non- | 1 | | | NP_00 | |
| subcomponent | | 5 | 3:Contig1 | | 6512.1 | 3 | | | | CDS | | | | 1725.1 | |
| | | | | | 2 | | | | | | | | | | |
| C1,S | C1S | AE111s1 | AE111_X | 573 | AC00 | 14860 | R | H | Exon4 | Missens | 1 | CGT | CAT | NP_00 | 119 |
| subcomponent | | 7 | 4:Contig1 | | 6512.1 | 1 | | | | e | | | | 1725.1 | |
| | | | | | 2 | | | | | | | | | | |
| C1,S | C1S | AE111s1 | AE111_X | 83 | AC00 | 14977 | | | Intron4 | Non- | 1 | | | NP_00 | |
| subcomponent | | 9 | 5:Contig1 | | 6512.1 | 2 | | | | CDS | | | | 1725.1 | |
| | | | | | 2 | | | | | | | | | | |
| C1,S | C1S | AE111s1 | AE111_X | 196 | AC00 | 14988 | C | C | Exon5 | Silent | 1 | TGG | TGT | NP_00 | 147 |
| subcomponent | | 8 | 5:Contig1 | | 6512.1 | 5 | | | | | | | | 1725.1 | |
| | | | | | 2 | | | | | | | | | | |
| C1,S | C1S | AE111s2 | AE111_X | 552 | AC00 | 15144 | N | K | Exon7 | Missens | 1 | AAT | AAA | NP_00 | 260 |
| subcomponent | | 0 | 7:Contig1 | | 6512.1 | 8 | | | | e | | | | 1725.1 | |
| | | | | | 2 | | | | | | | | | | |
| C1,S | C1S | AE111s2 | AE111_X | 180 | AC00 | 15213 | A | V | Exon8 | Missens | 1 | GCG | GTG | NP_00 | 307 |
| subcomponent | | 1 | 8:Contig1 | | 6512.1 | 5 | | | | e | | | | 1725.1 | |
| | | | | | 2 | | | | | | | | | | |
| C1,S | C1S | AE111s2 | AE111_X | 194 | AC00 | 15214 | V | I | Exon8 | Missens | 1 | GTC | ATC | NP_00 | 312 |
| subcomponent | | 2 | 8:Contig1 | | 6512.1 | 9 | | | | e | | | | 1725.1 | |
| | | | | | 2 | | | | | | | | | | |
| C1,S | C1S | AE111s5 | AE11_X | 74 | AC00 | 15308 | | | Intron9 | Non- | 1 | | | NP_00 | |
| subcomponent | | | 10:Contig | | 6512.1 | 6 | | | | CDS | | | | 1725.1 | |
| | | | 1 | | 2 | | | | | | | | | | |
| C1,S | C1S | AE111s6 | AE111_X | 50 | AC00 | 15413 | | | Intron1 | Non- | 1 | | | NP_00 | |
| subcomponent | | | 11:Contig | | 6512.1 | 7 | | | | CDS | | | | 1725.1 | |
| | | | 1 | | 2 | | | | | | | | | | |
| C1,S | C1S | AE111s8 | AE111_X | 407 | AC00 | 15583 | L | L | Exon12 | Silent | 1 | CTC | CTT | NP_00 | 559 |
| subcomponent | | | 12.f2:Con | | 6512.1 | 0 | | | | | | | | 1725.1 | |
| | | | tig1 | | 2 | | | | | | | | | | |
| alanyl | ANPEP | AE112s5 | AE112_X | 90 | AC01 | 79819 | | | 3FLANK | Non- | 0 | | | NP_00 | |
| aminopeptidase | | 7 | 20.f1:Con | | 8988.6 | | | | | CDS | | | | 1141.1 | |
| | | | tig1 | | | | | | | | | | | | |
| alanyl | ANPEP | AE112s5 | AE112_X | 354 | AC01 | 80083 | | | Exon20 | Non- | 0 | | | NP_00 | |
| aminopeptidase | | 5 | 20.f1:Con | | 8988.6 | | | | | CDS | | | | 1141.1 | |
| | | | tig1 | | | | | | | | | | | | |
| alanyl | ANPEP | AE112s5 | AE112_X | 465 | AC01 | 80194 | | | Exon20 | Non- | 0 | | | NP_00 | |
| aminopeptidase | | 6 | 20.f1:Con | | 8988.6 | | | | | CDS | | | | 1141.1 | |
| | | | tig1 | | | | | | | | | | | | |
| alanyl | ANPEP | AE112s5 | AE112_X | 395 | AC01 | 80295 | | | Exon20 | Non- | 0 | | | NP_00 | |
| aminopeptidase | | 8 | 20.f2:Con | | 8988.6 | | | | | CDS | | | | 1141.1 | |
| | | | tig2 | | | | | | | | | | | | |
| alanyl | ANPEP | AE112s4 | AE112_X | 24 | AC01 | 85340 | | | Intron19 | Non- | 0 | | | NP_00 | |
| aminopeptidase | | 4 | 19:Contig | | 8988.6 | | | | | CDS | | | | 1141.1 | |
| | | | 3 | | | | | | | | | | | | |
| alanyl | ANPEP | AE112s4 | AE112_X | 75 | AC01 | 85391 | | | Intron19 | Non- | 0 | | | NP_00 | |
| aminopeptidase | | 6 | 19:Contig | | 8988.6 | | | | | CDS | | | | 1141.1 | |
| | | | 3 | | | | | | | | | | | | |
| alanyl | ANPEP | AE112s4 | AE112_X | 104 | AC01 | 85420 | | | Intron19 | Non- | 0 | | | NP_00 | |
| aminopeptidase | | 2 | 19:Contig | | 8988.6 | | | | | CDS | | | | 1141.1 | |
| | | | 3 | | | | | | | | | | | | |
| alanyl | ANPEP | AE112s4 | AE112_X | 113 | AC01 | 85429 | | | Intron19 | Non- | 0 | | | NP_00 | |
| aminopeptidase | | 3 | 19:Contig | | 8988.6 | | | | | CDS | | | | 1141.1 | |
| | | | 3 | | | | | | | | | | | | |
| alanyl | ANPEP | AE112s4 | AE112_X | 309 | AC01 | 85626 | | | Intron18 | Non- | 0 | | | NP_00 | |
| aminopeptidase | | 1 | 19:Contig | | 8988.6 | | | | | CDS | | | | 1141.1 | |
| | | | 2 | | | | | | | | | | | | |
| alanyl | ANPEP | AE112s4 | AE112_X | 79 | AC01 | 85921 | | | Intron18 | Non- | 0 | | | NP_00 | |
| aminopeptidase | | 0 | 18:Contig | | 8988.6 | | | | | CDS | | | | 1141.1 | |
| | | | 2 | | | | | | | | | | | | |
| alanyl | ANPEP | AE112s3 | AE112_X | 211 | AC01 | 86015 | I | I | Exon18 | Silent | 0 | ATT | ATC | NP_00 | 871 |
| aminopeptidase | | 6 | 18:Contig | | 8988.6 | | | | | | | | | 1141.1 | |
| | | | 1 | | | | | | | | | | | | |
| alanyl | ANPEP | AE112s3 | AE112_X | 242 | AC01 | 86084 | T | T | Exon18 | Silent | 0 | ACC | ACA | NP_00 | 848 |
| aminopeptidase | | 8 | 18:Contig | | 8988.6 | | | | | | | | | 1141.1 | |
| | | | 2 | | | | | | | | | | | | |
| alanyl | ANPEP | AE112s3 | AE112_X | 106 | AC01 | 86120 | | | Intron17 | Non- | 0 | | | NP_00 | |
| aminopeptidase | | 5 | 18:Contig | | 8988.6 | | | | | CDS | | | | 1141.1 | |
| | | | 1 | | | | | | | | | | | | |
| alanyl | ANPEP | AE112s2 | AE112_X | 112 | AC01 | 87154 | | | Intron17 | Non- | 0 | | | NP_00 | |
| aminopeptidase | | 9 | 17:Contig | | 8988.6 | | | | | CDS | | | | 1141.1 | |
| | | | 1 | | | | | | | | | | | | |
| alanyl | ANPEP | AE112s3 | AE112_X | 204 | AC01 | 87261 | F | F | Exon17 | Silent | 0 | TTC | TTT | NP_00 | 811 |
| aminopeptidase | | 3 | 17:Contig | | 8988.6 | | | | | | | | | 1141.1 | |
| | | | 2 | | | | | | | | | | | | |
| alanyl | ANPEP | AE112s3 | AE112_X | 170 | AC01 | 87295 | A | V | Exon17 | Missens | 0 | GCT | GTT | NP_00 | 800 |
| aminopeptidase | | 2 | 17:Contig | | 8988.6 | | | | | e | | | | 1141.1 | |
| | | | 2 | | | | | | | | | | | | |
| alanyl | ANPEP | AE112s3 | AE112_X | 267 | AC01 | 87309 | T | T | Exon17 | Silent | 0 | ACC | ACT | NP_00 | 795 |
| aminopeptidase | | 0 | 17:Contig | | 8988.6 | | | | | | | | | 1141.1 | |
| | | | 1 | | | | | | | | | | | | |
| alanyl | ANPEP | AE112s2 | AE112_X | 145 | AC01 | 88148 | | | Intron14 | Non- | 0 | | | NP_00 | |
| aminopeptidase | | 6 | 15:Contig | | 8988.6 | | | | | CDS | | | | 1141.1 | |
| | | | 1 | | | | | | | | | | | | |
| alanyl | ANPEP | AE112s2 | AE112_X | 323 | AC01 | 94128 | | | Intron13 | Non- | 0 | | | NP_00 | |
| aminopeptidase | | 2 | 13:Contig | | 8988.6 | | | | | CDS | | | | 1141.1 | |
| | | | 1 | | | | | | | | | | | | |
| alanyl | ANPEP | AE112s2 | AE112_X | 101 | AC01 | 94163 | | | Intron13 | Non- | 0 | | | NP_00 | |
| aminopeptidase | | 3 | 13:Contig | | 8988.6 | | | | | CDS | | | | 1141.1 | |
| | | | 2 | | | | | | | | | | | | |
| alanyl | ANPEP | AE112s2 | AE112_X | 210 | AC01 | 94241 | | | Intron13 | Non- | 0 | | | NP_00 | |
| aminopeptidase | | 1 | 13:Contig | | 8988.6 | | | | | CDS | | | | 1141.1 | |
| | | | 1 | | | | | | | | | | | | |
| alanyl | ANPEP | AE112s1 | AE112_X | 294 | AC01 | 96042 | | | Intron10 | Non- | 0 | | | NP_00 | |
| aminopeptidase | | 7 | 11:Contig | | 8988.6 | | | | | CDS | | | | 1141.1 | |
| | | | 1 | | | | | | | | | | | | |
| alanyl | ANPEP | AE112s1 | AE112_X | 171 | AC01 | 96128 | I | M | Exon11 | Missens | 0 | ATA | ATG | NP_00 | 603 |
| aminopeptidase | | 8 | 11:Contig | | 8988.6 | | | | | e | | | | 1141.1 | |
| | | | 2 | | | | | | | | | | | | |
| alanyl | ANPEP | AE112s1 | AE112_X | 172 | AC01 | 96129 | I | K | Exon11 | Missens | 0 | ATA | AAA | NP_00 | 603 |
| aminopeptidase | | 9 | 11:Contig | | 8988.6 | | | | | e | | | | 1141.1 | |
| | | | 2 | | | | | | | | | | | | |
| alanyl | ANPEP | AE112s7 | AE112_X | 42 | AC01 | 98619 | | | Intron7 | Non- | 0 | | | NP_00 | |
| aminopeptidase | | 1 | 7:Contig1 | | 8988.6 | | | | | CDS | | | | 1141.1 | |
| alanyl | ANPEP | AE112s7 | AE112_X | 61 | AC01 | 98638 | | | Intron7 | Non- | 0 | | | NP_00 | |
| aminopeptidase | | 2 | 7:Contig1 | | 8988.6 | | | | | CDS | | | | 1141.1 | |
| alanyl | ANPEP | AE112s6 | AE112_X | 293 | AC01 | 98879 | | | Intron6 | Non- | 0 | | | NP_00 | |
| aminopeptidase | | 9 | 6:Contig1 | | 8988.6 | | | | | CDS | | | | 1141.1 | |
| alanyl | ANPEP | AE112s6 | AE112_X | 202 | AC01 | 99333 | F | F | Exon5 | Silent | 0 | TTC | TTT | NP_00 | 369 |
| aminopeptidase | | 8 | 5:Contig2 | | 8988.6 | | | | | | | | | 1141.1 | |
| alanyl | ANPEP | AE112s6 | AE112_X | 348 | AC01 | 99430 | | | Intron4 | Non- | 0 | | | NP_00 | |
| aminopeptidase | | 4 | 4:Contig1 | | 8988.6 | | | | | CDS | | | | 1141.1 | |
| alanyl | ANPEP | AE112s6 | AE112_X | 218 | AC01 | 99560 | T | T | Exon4 | Silent | 0 | ACG | ACA | NP_00 | 321 |
| aminopeptidase | | 3 | 4:Contig1 | | 8988.6 | | | | | | | | | 1141.1 | |
| alanyl | ANPEP | AE112s6 | AE112_X | 209 | AC01 | 99569 | L | L | Exon4 | Silent | 0 | CTG | CTT | NP_00 | 318 |
| aminopeptidase | | 2 | 4:Contig1 | | 8988.6 | | | | | | | | | 1141.1 | |
| alanyl | ANPEP | AE112s6 | AE112_X | 187 | AC01 | 99591 | A | V | Exon4 | Missens | 0 | GCG | GTG | NP_00 | 311 |
| aminopeptidase | | 1 | 4:Contig1 | | 8988.6 | | | | | e | | | | 1141.1 | |
| alanyl | ANPEP | AE112s6 | AE112_X | 81 | AC01 | 99697 | | | Intron3 | Non- | 0 | | | NP_00 | |
| aminopeptidase | | 5 | 4:Contig1 | | 8988.6 | | | | | CDS | | | | 1141.1 | |
| alanyl | ANPEP | AE112s5 | AE112_X | 253 | AC01 | 10031 | | | Intron2 | Non- | 0 | | | NP_00 | |
| aminopeptidase | | 3 | 2:Contig1 | | 8988.6 | 5 | | | | CDS | | | | 1141.1 | |
| alanyl | ANPEP | AE112s5 | AE112_X | 109 | AC01 | 10045 | T | T | Exon2 | Silent | 0 | ACT | ACA | NP_00 | 209 |
| aminopeptidase | | 2 | 2:Contig1 | | 8988.6 | 9 | | | | | | | | 1141.1 | |
| alanyl | ANPEP | AE112s1 | AE112_X | 94 | AC01 | 10095 | | | Intron1 | Non- | 0 | | | NP_00 | |
| aminopeptidase | | 3 | 1.f1Conti | | 8988.6 | 4 | | | | CDS | | | | 1141.1 | |
| | | | g1 | | | | | | | | | | | | |
| alanyl | ANPEP | AE112s1 | AE112_X | 474 | AC01 | 10123 | D | D | Exon1 | Silent | 0 | GAG | GAT | NP_00 | 118 |
| aminopeptidase | | 5 | 1.f2:Conti | | 8988.6 | 8 | | | | | | | | 1141.1 | |
| | | | g1 | | | | | | | | | | | | |
| alanyl | ANPEP | AE112s1 | AE112_X | 976 | AC01 | 10173 | | | 5FLANK | Non- | 0 | | | NP_00 | |
| aminopeptidase | | | 0.f1:Conti | | 8988.6 | 7 | | | | CDS | | | | 1141.1 | |
| | | | g1 | | | | | | | | | | | | |
| alanyl | ANPEP | AE112s5 | AE112_X | 532 | AC01 | 10218 | | | 5FLANK | Non- | 0 | | | NP_00 | |
| aminopeptidase | | | 0.f2:Conti | | 8988.6 | 0 | | | | CDS | | | | 1141.1 | |
| | | | g1 | | | | | | | | | | | | |
| alanyl | ANPEP | AE112s4 | AE112_X | 463 | AC01 | 10224 | | | 5FLANK | Non- | 0 | | | NP_00 | |
| aminopeptidase | | | 0.f2:Conti | | 8988.6 | 9 | | | | CDS | | | | 1141.1 | |
| | | | g1 | | | | | | | | | | | | |
| alanyl | ANPEP | AE112s3 | AE112_X | 337 | AC01 | 10237 | | | 5FLANK | Non- | 0 | | | NP_00 | |
| aminopeptidase | | | 0.f2:Conti | | 8988.6 | 5 | | | | CDS | | | | 1141.1 | |
| | | | g1 | | | | | | | | | | | | |
| alanyl | ANPEP | AE112s2 | AE112_{—X} | 316 | AC01 | 10239 | | | 5FLANK | Non- | 0 | | | NP_00 | |
| aminopeptidase | | | 0.f2:Conti | | 8988.6 | 6 | | | | CDS | | | | 1141.1 | |
| | | | g1 | | | | | | | | | | | | |
| alanyl | ANPEP | AE112s9 | AE112_X | 779 | AC01 | 10249 | | | 5FLANK | Non- | 0 | | | NP_00 | |
| aminopeptidase | | | 0.f3:Conti | | 8988.6 | 0 | | | | CDS | | | | 1141.1 | |
| | | | g2 | | | | | | | | | | | | |
| alanyl | ANPEP | AE112s6 | AE112_X | 511 | AC01 | 10258 | | | 5FLANK | Non- | 0 | | | NP_00 | |
| aminopeptidase | | | 0.f3 Conti | | 8988.6 | 3 | | | | CDS | | | | 1141.1 | |
| | | | g1 | | | | | | | | | | | | |
| alanyl | ANPEP | AE112s7 | AE112_X | 561 | AC01 | 10263 | | | 5FLANK | Non- | 0 | | | NP_00 | |
| aminopeptidase | | | 0.f3:Conti | | 8988.6 | 3 | | | | CDS | | | | 1141.1 | |
| | | | g1 | | | | | | | | | | | | |
| alanyl | ANPEP | AE112s1 | AE112_X | 954 | AC01 | 10266 | | | 5FLANK | Non- | 0 | | | NP_00 | |
| aminopeptidase | | 1 | 0.f3:Conti | | 8988.6 | 5 | | | | CDS | | | | 1141.1 | |
| | | | g2 | | | | | | | | | | | | |
| alanyl | ANPEP | AE112s1 | AE112_X | 955 | AC01 | 10266 | | | 5FLANK | Non- | 0 | | | NP_00 | |
| aminopeptidase | | 2 | 0.f3:Conti | | 8988.6 | 6 | | | | CDS | | | | 1141.1 | |
| | | | g2 | | | | | | | | | | | | |
| meprin A, beta | MEP1B | AE113s4 | AE113_X | 949 | AC01 | 56518 | | | 5FLANK | Non- | 1 | | | NP_00 | |
| | | | 0.f1:Conti | | 5563.5 | | | | | CDS | | | | 5916.1 | |
| | | | g2 | | | | | | | | | | | | |
| meprin A, beta | MEP1B | AE113s3 | AE113_X | 876 | AC01 | 56591 | | | 5FLANK | Non- | 1 | | | NP_00 | |
| | | | 0.f1:Conti | | 5563.5 | | | | | CDS | | | | 5916.1 | |
| | | | g2 | | | | | | | | | | | | |
| meprin A, beta | MEP1B | AE113s2 | AE113_X | 828 | AC01 | 56639 | | | 5FLANK | Non- | 1 | | | NP_00 | |
| | | | 0.f1:Conti | | 5563.5 | | | | | CDS | | | | 5916.1 | |
| | | | g2 | | | | | | | | | | | | |
| meprin A, beta | MEP1B | AE113s1 | AE113_X | 803 | AC01 | 56664 | | | 5FLANK | Non- | 1 | | | NP_00 | |
| | | | 0.f1:Conti | | 5563.5 | | | | | CDS | | | | 5916.1 | |
| | | | g2 | | | | | | | | | | | | |
| meprin A, beta | MEP1B | AE113s5 | AE113_X | 615 | AC01 | 57081 | | | 5FLANK | Non- | 1 | | | NP_00 | |
| | | | 0.f2:Conti | | 5563.5 | | | | | CDS | | | | 5916.1 | |
| | | | g2 | | | | | | | | | | | | |
| meprin A, beta | MEP1B | AE113s3 | AE113_X | 71 | AC01 | 60239 | E | E | Exon3 | Silent | 1 | GAG | GAA | NP_00 | 50 |
| | | 4 | 3:Contig1 | | 5563.5 | | | | | | | | | 5916.1 | |
| meprin A, beta | MEP1B | AE113s3 | AE113_X | 66 | AC01 | 63013 | | | Intron5 | Non- | 1 | | | NP_00 | |
| | | 7 | 5:Contig1 | | 5563.5 | | | | | CDS | | | | 5916.1 | |
| meprin A, beta | MEP1B | AE113s3 | AE113_X | 409 | AC01 | 70436 | I | M | Exon6 | Missens | 1 | ATA | ATG | NP_00 | 115 |
| | | 8 | 6:Contig1 | | 5563.5 | | | | | e | | | | 5916.1 | |
| meprin A, beta | MEP1B | AE113s3 | AE113_X | 324 | AC01 | 71587 | | | Intron6 | Non- | 1 | | | NP_00 | |
| | | 9 | 7:Contig1 | | 5563.5 | | | | | CDS | | | | 5916.1 | |
| meprin A, beta | MEP1B | AE113s4 | AE113_X | 82 | AC01 | 71829 | | | Intron7 | Non- | 1 | | | NP_00 | |
| | | 1 | 7:Contig1 | | 5563.5 | | | | | CDS | | | | 5916.1 | |
| meprin A, beta | MEP1B | AE113s4 | AE113_X | 58 | AC01 | 71853 | | | Intron7 | Non- | 1 | | | NP_00 | |
| | | 0 | 7:Contig1 | | 5563.5 | | | | | CDS | | | | 5916.1 | |
| meprin A, beta | MEP1B | AE113s4 | AE113_X | 275 | AC01 | 75591 | S | S | Exon9 | Silent | 1 | TCA | TCG | NP_00 | 264 |
| | | 3 | 9:Contig1 | | 5563.5 | | | | | | | | | 5916.1 | |
| meprin A, beta | MEP1B | AE113s4 | AE113_X | 25 | AC01 | 75841 | | | Intron9 | Non- | 1 | | | NP_00 | |
| | | 2 | 9:Contig1 | | 5563.5 | | | | | CDS | | | | 5916.1 | |
| meprin A, beta | MEP1B | AE113s7 | AE113_X | 343 | AC01 | 77941 | | | Intron9 | Non- | 1 | | | NP_00 | |
| | | | 10:Contig | | 5563.5 | | | | | CDS | | | | 5916.1 | |
| meprin A, beta | MEP1B | AE113s6 | AE113_X | 281 | AC01 | 78003 | V | M | Exon10 | Missens | 1 | GTG | ATG | NP_00 | 326 |
| | | | 10:Contig | | 5563.5 | | | | | e | | | | 5916.1 | |
| meprin A, beta | MEP1B | AE113s9 | AE113_X | 358 | AC01 | 80648 | S | T | Exon10 | Missens | 1 | TCT | ACT | NP_00 | 408 |
| | | | 11.f1:Con | | 5563.5 | | | | | e | | | | 5916.1 | |
| | | | tig1 | | | | | | | | | | | | |
| meprin A, beta | MEP1B | AE113s8 | AE113_X | 311 | AC01 | 80695 | S | S | Exon10 | Silent | 1 | TCG | TCA | NP_00 | 423 |
| | | | 11.f1:Con | | 5563.5 | | | | | | | | | 5916.1 | |
| | | | tig1 | | | | | | | | | | | | |
| meprin A, beta | MEP1B | AE113s1 | AE113_X | 212 | AC01 | 82642 | H | P | Exon12 | Missens | 1 | CAC | CCC | NP_00 | 565 |
| | | 8 | 12:Contig | | 5563.5 | | | | | e | | | | 5916.1 | |
| | | | 3 | | | | | | | | | | | | |
| meprin A, beta | MEP1B | AE113s1 | AE113_X | 207 | AC01 | 82643 | H | H | Exon12 | Silent | 1 | CAC CAT | NP_00 | 565 | |
| | | 4 | 12:Contig | | 5563.5 | | | | | | | | | 5916.1 | |
| | | | 2 | | | | | | | | | | | | |
| meprin A, beta | MEP1B | AE113s1 | AE113_X | 107 | AC01 | 82747 | | | Intron12 | Non- | 1 | | | NP_00 | |
| | | 6 | 12:Contig | | 5563.5 | | | | | CDS | | | | 5916.1 | |
| | | | 3 | | | | | | | | | | | | |
| meprin A, beta | MEP1B | AE113s2 | AE113_X | 46 | AC01 | 82808 | | | Intron12 | Non- | 1 | | | NP_00 | |
| | | 1 | 12:Contig | | 5563.5 | | | | | CDS | | | | 5916.1 | |
| | | | 3 | | | | | | | | | | | | |
| meprin A, beta | MEP1B | AE113s2 | AE113_X | 285 | AC01 | 84369 | | | Intron12 | Non- | 1 | | | NP_00 | |
| | | 2 | 13:Contig | | 5563.5 | | | | | CDS | | | | 5916.1 | |
| | | | 2 | | | | | | | | | | | | |
| meprin A, beta | MEP1B | AE113s2 | AE113_X | 147 | AC01 | 85243 | D | D | Exon14 | Silent | 1 | GAC | GAT | NP_00 | 651 |
| | | 4 | 14:Contig | | 5563.5 | | | | | | | | | 5916.1 | |
| | | | 1 | | | | | | | | | | | | |
| meprin A, beta | MEP1B | AE113s2 | AE113_X | 278 | AC01 | 85374 | L | P | Exon14 | Missens | 1 | CTG | CCG | NP_00 | 695 |
| | | 5 | 14:Contig | | 5563.5 | | | | | e | | | | 5916.1 | |
| | | | 1 | | | | | | | | | | | | |
| meprin A, beta | MEP1B | AE113s2 | AE113_X | 315 | AC01 | 85411 | | | Intron14 | Non- | 1 | | | NP_00 | |
| | | 6 | 14:Contig | | 5563.5 | | | | | CDS | | | | 5916.1 | |
| | | | 1 | | | | | | | | | | | | |
| meprin A, beta | MEP1B | AE113s3 | AE113_X | 25 | AC01 | 87572 | | | Intron14 | Non- | 1 | | | NP_00 | |
| | | 1 | 15:Contig | | 5563.5 | | | | | CDS | | | | 5916.1 | |
| | | | 2 | | | | | | | | | | | | |
| meprin A, beta | MEP1B | AE113s3 | AE113_X | 572 | AC01 | 87777 | | | Exon15 | Non- | 1 | | | NP_00 | |
| | | 0 | 15:Contig | | 5563.5 | | | | | CDS | | | | 5916.1 | |
| | | | 1 | | | | | | | | | | | | |
| Aminopeptidase | XPNPE | AE114s3 | AE114_X | 208 | AL35 | 77943 | T | T | Exon19 | Silent | 0 | ACC | ACG | NP_00 | 591 |
| P-like | PL | 0 | 19:Contig | | 4951.6 | | | | | | | | | 6514.1 | |
| | | | 1 | | | | | | | | | | | | |
| Aminopeptidase | XPNPE | AE114s3 | AE114_X | 64 | AL35 | 78087 | | | Intron18 | Non- | 0 | | | NP_00 | |
| P-like | PL | 1 | 19:Contig | | 4951.6 | | | | | CDS | | | | 6514.1 | |
| | | | 1 | | | | | | | | | | | | |
| Aminopeptidase | XPNPE | AE114s2 | AE114_X | 57 | AL35 | 83332 | | | Intron16 | Non- | 0 | | | NP_00 | |
| P-like | PL | 9 | 16:Contig | | 4951.6 | | | | | CDS | | | | 6514.1 | |
| | | | 1 | | | | | | | | | | | | |
| Aminopeptidase | XPNPE | AE114s2 | AE114_X | 299 | AL35 | 83574 | | | Intron15 | Non- | 0 | | | NP_00 | |
| P-like | PL | 8 | 16:Contig | | 4951.6 | | | | | CDS | | | | 6514.1 | |
| | | | 1 | | | | | | | | | | | | |
| Aminopeptidase | XPNPE | AE114s2 | AE114_X | 41 | AL35 | 84567 | | | Intron14 | Non- | 0 | | | NP_00 | |
| P-like | PL | 6 | 15:Contig | | 4951.6 | | | | | CDS | | | | 6514.1 | |
| | | | 1 | | | | | | | | | | | | |
| Aminopeptidase | XPNPE | AE114s2 | AE114_X | 243 | AL35 | 85927 | | | Intron14 | Non- | 0 | | | NP_00 | |
| P-like | PL | 5 | 14:Contig | | 4951.6 | | | | | CDS | | | | 6514.1 | |
| | | | 1 | | | | | | | | | | | | |
| Aminopeptidase | XPNPE | AE114s2 | AE114_X | 241 | AL35 | 85929 | | | Intron14 | Non- | 0 | | | NP_00 | |
| P-like | PL | 4 | 14:Contig | | 4951.6 | | | | | CDS | | | | 6514.1 | |
| | | | 1 | | | | | | | | | | | | |
| Aminopeptidase | XPNPE | AE114s2 | AE114_X | 365 | AL35 | 88001 | | | Intron13 | Non- | 0 | | | NP_00 | |
| P-like | PL | 3 | 13:Contig | | 4951.6 | | | | | CDS | | | | 6514.1 | |
| | | | 1 | | | | | | | | | | | | |
| Aminopeptidase | XPNPE | AE114s2 | AE114_X | 252 | AL35 | 88114 | | | Intron13 | Non- | 0 | | | NP_00 | |
| P-like | PL | 2 | 13:Contig | | 4951.6 | | | | | CDS | | | | 6514.1 | |
| | | | 1 | | | | | | | | | | | | |
| Aminopeptidase | XPNPE | AE114s1 | AE114_X | 183 | AL35 | 90430 | | | Intron12 | Non- | 0 | | | NP_00 | |
| P-like | PL | 8 | 12:Contig | | 4951.6 | | | | | CDS | | | | 6514.1 | |
| | | | 1 | | | | | | | | | | | | |
| Aminopeptidase | XPNPE | AE114s1 | AE114_X | 347 | AL3S | 90537 | | | Intron11 | Non- | 0 | | | NP_00 | |
| P-like | PL | 4 | 11:Contig | | 4951.6 | | | | | CDS | | | | 6514.1 | |
| | | | 1 | | | | | | | | | | | | |
| Aminopeptidase | XPNPE | AE114s1 | AE114_X | 240 | AL35 | 90644 | | | Intron11 | Non- | 0 | | | NP_00 | |
| P-like | PL | 3 | 11:Contig | | 4951.6 | | | | | CDS | | | | 6514.1 | |
| | | | 1 | | | | | | | | | | | | |
| Aminopeptidase | XPNPE | AE114s1 | AE114_X | 367 | AL35 | 90816 | | | Intron10 | Non- | 0 | | | NP_00 | |
| P-like | PL | 6 | 11:Contig | | 4951.6 | | | | | CDS | | | | 6514.1 | |
| | | | 2 | | | | | | | | | | | | |
| Aminopeptidase | XPNPE | AE114s1 | AE114_X | 392 | AL35 | 92632 | | | Intron10 | Non- | 0 | | | NP_00 | |
| P-like | PL | 2 | 10:Contig | | 4951.6 | | | | | CDS | | | | 6514.1 | |
| | | | 1 | | | | | | | | | | | | |
| Aminopeptidase | XPNPE | AE114s4 | AE114_X | 47 | AL35 | 93405 | | | Intron9 | Non- | 0 | | | NP_00 | |
| P-like | PL | 5 | 9:Contig2 | | 4951.6 | | | | | CDS | | | | 6514.1 | |
| Aminopeptidase | XPNPE | AE114s4 | AE114_X | 55 | AL35 | 93604 | | | Intron8 | Non- | 0 | | | NP_00 | |
| P-like | PL | 4 | 9:Contig1 | | 4951.6 | | | | | CDS | | | | 6514.1 | |
| Aminopeptidase | XPNPE | AE114s4 | AE114_X | 355 | AL3 | 595319 | | | Intron7 | Non- | 0 | | | NP_00 | |
| P-like | PL | 3 | 8:Contig1 | | 4951.6 | | | | | CDS | | | | 6514.1 | |
| Aminopeptidase | XPNPE | AE114s4 | AE114_X | 562 | AL35 | 96650 | | | Intron7 | Non- | 0 | | | NP_00 | |
| P-like | PL | 2 | 7:Contig1 | | 4951.6 | | | | | CDS | | | | 6514.1 | |
| Aminopeptidase | XPNPE | AE114s4 | AE114_X | 107 | AL35 | 99049 | | | Intron5 | Non- | 0 | | | NP_00 | |
| P-like | PL | 1 | 6:Contig1 | | 4951.6 | | | | | CDS | | | | 6514.1 | |
| Aminopeptidase | XPNPE | AE114s3 | AE114_X | 311 | AL35 | 10063 | | | Intron5 | Non- | 0 | | | NP_00 | |
| P-like | PL | 5 | 5:Contig2 | | 4951.6 | 6 | | | | CDS | | | | 6514.1 | |
| Aminopeptidase | XPNPE | AE114s3 | AE114_X | 307 | AL35 | 10064 | | | Intron5 | Non- | 0 | | | NP_00 | |
| P-like | PL | 4 | 5:Contig2 | | 4951.6 | 0 | | | | CDS | | | | 6514.1 | |
| Aminopeptidase | XPNPE | AE114s3 | AE114_X | 39 | AL35 | 10090 | | | Intron4 | Non- | 0 | | | NP_00 | |
| P-like | PL | 6 | 5:Contig2 | | 4951.6 | 8 | | | | CDS | | | | 6514.1 | |
| Aminopeptidase | XPNPE | AE114s3 | AE114_X | 324 | AL35 | 10091 | | | Intron4 | Non- | 0 | | | NP_00 | |
| P-like | PL | 8 | 5:Contig3 | | 4951.6 | 0 | | | | CDS | | | | 6514.1 | |
| Aminopeptidase | XPNPE | AE114s3 | AE114_X | 183 | AL35 | 10444 | D | D | Exon3 | Silent | 0 | GAC | GAT | NP_00 | 75 |
| P-like | PL | 3 | 3:Contig2 | | 4951.6 | 3 | | | | | | | | 6514.1 | |
| Aminopeptidase | XPNPE | AE114s3 | AE114_X | 138 | AL35 | 10580 | | | Intron1 | Non- | 0 | | | NP_00 | |
| P-like | PL | 2 | 2:Contig1 | | 4951.6 | 4 | | | | CDS | | | | 6514.1 | |
| Aminopeptidase | XPNPE | AE114s5 | AE114_X | 796 | AL35 | 12051 | | | 5FLANK | Non- | 0 | | | NP_00 | |
| P-like | PL | | 0.f1:Conti | | 4951.6 | 6 | | | | CDS | | | | 6514.1 | |
| | | | g1 | | | | | | | | | | | | |
| Aminopeptidase | XPNPE | AE114s3 | AE114_X | 504 | AL35 | 12080 | | | 5FLANK | Non- | 0 | | | NP_00 | |
| P-like | PL | | 0.f1:Conti | | 4951.6 | 8 | | | | CDS | | | | 6514.1 | |
| | | | g1 | | | | | | | | | | | | |
| Aminopeptidase | XPNPE | AE114s7 | AE114_X | 631 | AL35 | 12083 | | | 5FLANK | Non- | 0 | | | NP_00 | |
| P-like | PL | | 0.f2:Conti | | 4951.6 | 7 | | | | CDS | | | | 6514.1 | |
| | | | g1 | | | | | | | | | | | | |
| Aminopeptidase | XPNPE | AE114s6 | AE114_X | 473 | AL35 | 12099 | | | 5FLANK | Non- | 0 | | | NP_00 | |
| P-like | PL | | 0.f2:Conti | | 4951.6 | 5 | | | | CDS | | | | 6514.1 | |
| | | | g1 | | | | | | | | | | | | |
| Aminopeptidase | XPNPE | AE114s9 | AE114_X | 211 | AL35 | 12137 | | | 5FLANK | Non- | 0 | | | NP_00 | |
| P-like | PL | | 0.f3:Conti | | 4951.6 | 2 | | | | CDS | | | | 6514.1 | |
| | | | g1 | | | | | | | | | | | | |
| Tissue kallikrein | KLK1 | AE115s1 | AE115_X | 310 | AF243 | 7613 | | | Intron4 | Non- | 0 | | | NP_00 | |
| | | 3 | 4:Contig2 | | 527.1 | | | | | CDS | | | | 2248.1 | |
| Tissue kallikrein | KLK1 | AE115s1 | AE115_X | 309 | AF243 | 7614 | | | Intron4 | Non- | 0 | | | NP_00 | |
| | | 2 | 4:Contig2 | | 527.1 | | | | | CDS | | | | 2248.1 | |
| Tissue kallikrein | KLK1 | AE115s1 | AE115_X | 189 | AF243 | 7734 | H | H | Exon4 | Silent | 0 | CAC | CAT | NP_00 | 189 |
| | | 1 | 4:Contig2 | | 527.1 | | | | | | | | | 2248.1 | |
| Tissue kallikrein | KLK1 | AE115s1 | AE115_X | 58 | AF243 | 7865 | | | Intron3 | Non- | 0 | | | NP_00 | |
| | | 4 | 4:Contig2 | | 527.1 | | | | | CDS | | | | 2248.1 | |
| Tissue kallikrein | KLK1 | AE115s1 | AE115_X | 333 | AF243 | 12221 | | | 5FLANK | Non- | 0 | | | NP_00 | |
| | | | 0.f2:Conti | | 527.1 | | | | | CDS | | | | 2248.1 | |
| | | | g1 | | | | | | | | | | | | |
| Tissue kallikrein | KLK1 | AE115s4 | AE115_X | 415 | AF243 | 12270 | | | 5FLANK | Non- | 0 | | | NP_00 | |
| | | | 0.f3:Conti | | 527.1 | | | | | CDS | | | | 2248.1 | |
| | | | g4 | | | | | | | | | | | | |
| Tissue kallikrein | KLK1 | AE115s3 | AE115_X | 203 | AF243 | 12482 | | | 5FLANK | Non- | 0 | | | NP_00 | |
| | | | 0.f3:Conti | | 527.1 | | | | | CDS | | | | 2248.1 | |
| | | | g4 | | | | | | | | | | | | |
| Aminopeptidase | XPNPE | AE116s1 | AE116_X | 1006 | AL02 | 53154 | | | 5FLANK | Non- | 1 | | | NP_00 | |
| P (membrane | P2 | | 0.f1:Conti | | 3653.1 | | | | | CDS | | | | 3390.2 | |
| bound) | | | g2 | | | | | | | | | | | | |
| Aminopeptidase | XPNPE | AE116s2 | AE116_X | 875 | AL02 | 54031 | | | 5FLANK | Non- | 1 | | | NP_00 | |
| P (membrane | P2 | | 0.f3:Conti | | 3653.1 | | | | | CDS | | | | 3390.2 | |
| bound) | | | g1 | | | | | | | | | | | | |
| Aminopeptidase | XPNPE | AE116s4 | AE116_X | 144 | AL02 | 54334 | | | Exon1 | Non- | 1 | | | NP_00 | |
| P (membrane | P2 | | 1.f1:Conti | | 3653.1 | | | | | CDS | | | | 3390.2 | |
| bound) | | | g1 | | | | | | | | | | | | |
| Aminopeptidase | XPNPE | AE116s3 | AE116_X | 105 | AL02 | 54373 | | | Exon1 | Non- | 1 | | | NP_00 | |
| P(membrane | P2 | | 1.f1:Conti | | 3653.1 | | | | | CDS | | | | 3390.2 | |
| bound) | | | g1 | | | | | | | | | | | | |
| Aminopeptidase | XPNPE | AE116s2 | AE116_X | 319 | AL02 | 60282 | | | Intron3 | Non- | 1 | | | NP_00 | |
| P(membrane | P2 | 9 | 4:Contig1 | | 3653.1 | | | | | CDS | | | | 3390.2 | |
| bound) | | | | | | | | | | | | | | | |
| Aminopeptidase | XPNPE | AE116s3 | AE116_X | 557 | AL02 | 61440 | | | Intron4 | Non- | 1 | | | NP_00 | |
| P (membrane | P2 | 0 | 5:Contig2 | | 3653.1 | | | | | CDS | | | | 3390.2 | |
| bound) | | | | | | | | | | | | | | | |
| Aminopeptidase | XPNPE | AE116s3 | AE116_X | 375 | AL02 | 61976 | | | Intron6 | Non- | 1 | | | NP_00 | |
| P (membrane | P2 | 2 | 6:Contig1 | | 3653.1 | | | | | CDS | | | | 3390.2 | |
| bound) | | | | | | | | | | | | | | | |
| Aminopeptidase | XPNPE | AE116s3 | AE116_X | 93 | AL02 | 62748 | | | Intron6 | Non- | 1 | | | NP_00 | |
| P (membrane | P2 | 3 | 7:Contig1 | | 3653.1 | | | | | CDS | | | | 3390.2 | |
| bound) | | | | | | | | | | | | | | | |
| Aminopeptidase | XPNPE | AE116s7 | AE116_X | 306 | AL02 | 67566 | | | Intron10 | Non- | 1 | | | NP_00 | |
| P (membrane | P2 | | 10:Contig | | 3653.1 | | | | | CDS | | | | 3390.2 | |
| bound) | | | 1 | | | | | | | | | | | | |
| Aminopeptidase | XPNPE | AE116s1 | AE116_X | 186 | AL02 | 83578 | E | E | Exon21 | Silent | 1 | GAG | GAA | NP_00 | 636 |
| P(membrane | P2 | 8 | 21.f1:Con | | 3653.1 | | | | | | | | | 3390.2 | |
| bound) | | | tig1 | | | | | | | | | | | | |
| Aminopeptidase | XPNPE | AE116s2 | AE116_X | 351 | AL02 | 83851 | | | Intron21 | Non- | 1 | | | NP_00 | |
| P (membrane | P2 | 8 | 21.f4:Con | | 3653.1 | | | | | CDS | | | | 3390.2 | |
| bound) | | | tig4 | | | | | | | | | | | | |
| Aminopeptidase | XPNPE | AE116s2 | AE116_X | 1206 | AL02 | 84706 | | | Intron21 | Non- | 1 | | | NP_00 | |
| P (membrane | P2 | 1 | 21.f3:Con | | 3653.1 | | | | | CDS | | | | 3390.2 | |
| bound) | | | tig1 | | | | | | | | | | | | |
| Soluble | GUCY | AE117s1 | AE117_X | 368 | AP000 | 39950 | A | A | Exon6 | Silent | 1 | GCC | GCT | NP_00 | 593 |
| guanylate | 1A2 | 1 | 6:Contig2 | | 906.3 | | | | | | | | | 0846.1 | |
| cyclase 1, alpha- | | | | | | | | | | | | | | | |
| 2 subunit | | | | | | | | | | | | | | | |
| Soluble | GUCY | AE117s1 | AE117_X | 65 | AP000 | 55148 | | | Intron7 | Non- | 1 | | | NP_00 | |
| guanylate | 1A2 | 3 | 7:Contig2 | | 906.3 | | | | | CDS | | | | 0846.1 | |
| cyclase 1, alpha- | | | | | | | | | | | | | | | |
| 2 subunit | | | | | | | | | | | | | | | |
| Soluble | GUCY | AE117s7 | AE117_X | 561 | AC01 | 82689 | K | K | Exon3 | Silent | 0 | AAA | AAG | NP_00 | 145 |
| guanylate | 1A2 | | 3:Contig2 | | 9359.3 | | | | | | | | | 0846.1 | |
| cyclase 1, alpha- | | | | | | | | | | | | | | | |
| 2 subunit | | | | | | | | | | | | | | | |
| Soluble | GUCY | AE117s6 | AE117_X | 163 | AC01 | 82692 | N | N | Exon3 | Silent | 0 | AAC | AAT | NP_00 | 144 |
| guanylate | 1A2 | | 3:Contig2 | | 9359.3 | | | | | | | | | 0846.1 | |
| cyclase 1, alpha- | | | | | | | | | | | | | | | |
| 2 subunit | | | | | | | | | | | | | | | |
| Soluble | GUCY | AE117s1 | AE117_X | 74 | AC01 | 82781 | | | Intron2 | Non- | 0 | | | NP_00 | |
| guanylate | 1A2 | 0 | 3:Contig2 | | 9359.3 | | | | | CDS | | | | 0846.1 | |
| cyclase 1, alpha- | | | | | | | | | | | | | | | |
| 2 subunit | | | | | | | | | | | | | | | |
| Soluble | GUCY | AE117s9 | AE117_X | 684 | AC01 | 82812 | | | Intron2 | Non- | 0 | | | NP_00 | |
| guanylate | 1A2 | | 3:Contig2 | | 9359.3 | | | | | CDS | | | | 0846.1 | |
| cyclase 1, alpha- | | | | | | | | | | | | | | | |
| 2 subunit | | | | | | | | | | | | | | | |
| Soluble | GUCY | AE117s1 | AE117_X | 595 | AP000 | 16260 | | | Intron7 | Non- | 1 | | | NP_00 | |
| guanylate | 1A2 | 4 | 8.f1:Conti | | 906.3 | 5 | | | | CDS | | | | 0846.1 | |
| cyclase 1, alpha- | | | g2 | | | | | | | | | | | | |
| 2 subunit | | | | | | | | | | | | | | | |
| Soluble | GUCY | AE117s1 | AE117_X | 501 | AP000 | 16312 | | | Exon8 | Non- | 1 | | | NP_00 | |
| guanylate | 1A2 | 5 | 8.f2:Conti | | 906.3 | 7 | | | | CDS | | | | 0846.1 | |
| cyclase 1, alpha- | | | g1 | | | | | | | | | | | | |
| 2 subunit | | | | | | | | | | | | | | | |
| Soluble | GUCY | AE117s1 | AE117_X | 217 | AC01 | 16837 | | | 5FLANK | Non- | 1 | | | NP_00 | |
| guanylate | 1A2 | | 0.f1:Conti | | 9359.3 | 6 | | | | CDS | | | | 0846.1 | |
| cyclase 1, alpha- | | | g1 | | | | | | | | | | | | |
| 2 subunit | | | | | | | | | | | | | | | |
| Soluble | GUCY | AE117s2 | AE117_X | 246 | AC01 | 16840 | | | 5FLANK | Non- | 1 | | | NP_00 | |
| guanylate | 1A2 | | 0.f1:Conti | | 9359.3 | 5 | | | | CDS | | | | 0846.1 | |
| cyclase 1, alpha- | | | g1 | | | | | | | | | | | | |
| 2 subunit | | | | | | | | | | | | | | | |
| Soluble | GUCY | AE117s3 | AE117_X | 323 | AC01 | 16848 | | | 5FLANK | Non- | 1 | | | NP_00 | |
| guanylate | 1A2 | | 0.f1:Conti | | 9359.3 | 2 | | | | CDS | | | | 0846.1 | |
| cyclase 1, alpha- | | | g1 | | | | | | | | | | | | |
| 2 subunit | | | | | | | | | | | | | | | |

TABLE IV

| | | | | | PCR_LEFT_PRIMER (SEQ | | PCR_RIGHT_PRIMER (SEQ |
|---|---|---|---|---|---|---|---|
| | | | | | ID | | ID |
| GENE_{—DESCRIPTION} | HGNC_ID | SNP_ID | PCR_AMPLICON_NAME | PCR_LEFT_PRIMER | NO:) | PCR_RIGHT_PRIMER | NO:) |
| C1, S subcomponent | C1S | AE111s1 | AE111_X0.f1 | TGTAAAACGACGGCCAGTAGCC | 295 | CAGGAAACAGCTATGACCTCCTC | 442 |
| | | | | AACTAAAGGGGCATAAA | | CTCTCCCTCCCTTTAT | |
| C1, S subcomponent | C1S | AE111s2 | AE111_X0.f3 | TGTAAAACGACGGCCAGTCTGG | 296 | CAGGAAACAGCTATGACCTTCA | 443 |
| | | | | ACTTTCTGGGTTTGGTT | | GAAGCTGGTAGGAGCAA | |
| C1, S subcomponent | C1S | AE111s13 | AE111_X2 | TGTAAAACGACGGCCAGTCTTT | 297 | CAGGAAACAGCTATGACCCATG | 444 |
| | | | | GAAGGTGACACTGAGCC | | ACGCTGGGCTAATTTT | |
| C1, S subcomponent | C1S | AE111s15 | AE111_X3 | TGTAAAACGACGGCCAGTTTCTT | 298 | CAGGAAACAGCTATGACCCACT | 445 |
| | | | | CTGCCTCTGGCAAATA | | CCCTCCCACATCCTTAT | |
| C1, S subcomponent | C1S | AE111s17 | AE111_X4 | TGTAAAACGACGGCCAGTGTGC | 299 | CAGGAAACAGCTATGACCCCTC | 446 |
| | | | | CACCTGTACTCACAGTG | | ATTCAATGTGTTGCTCA | |
| C1, S subcomponent | C1S | AE111s19 | AE111_X5 | TGTAAAACGACGGCCAGTCGGA | 300 | CAGGAAACAGCTATGACCAAGA | 447 |
| | | | | TCTTTAAGCAATAGGCC | | AGTGCCAAATGAAGGCT | |
| C1, S subcomponent | C1S | AE111s18 | AE111_X5 | TGTAAAACGACGGCCAGTCGGA | 301 | CAGGAAACAGCTATGACCAAGA | 448 |
| | | | | TCTTTAAGCAATAGGC | | AGTGCCAAATGAAGGCT | |
| C1, S subcomponent | C1S | AE111s20 | AE111_X7 | TGTAAAACGACGGCCAGTTTTGT | 302 | CAGGAAACAGCTATGACCCATC | 449 |
| | | | | ATCTCCCACTTCCCAA | | TACCTGCTTTGATCCCA | |
| C1, S subcomponent | C1S | AE111s21 | AE111_X8 | TGTAAAACGACGGCCAGTTGAG | 303 | CAGGAAACAGCTATGACCCTCC | 450 |
| | | | | GACAACTGGACGATTTT | | ATGACTCACAAGGGAGA | |
| C1, S subcomponent | C1S | AE111s22 | AE111_X8 | TGTAAAACGACGGCCAGTTGAG | 304 | CAGGAAACAGCTATGACCCTCC | 451 |
| | | | | GACAACTGGACGATTTT | | ATGACTCACAAGGGAGA | |
| C1, S subcomponent | C1S | AE111s5 | AE111_X10 | TGTAAAACGACGGCCAGTAGAG | 305 | CAGGAAACAGCTATGACCTCTCC | 452 |
| | | | | CTGAGAGATGCCAGTTG | | AAACTCTGTCTCTGGC | |
| C1, S subcomponent | C1S | AE111s6 | AE111_X11 | TGTAAAACGACGGCCAGTGAGA | 306 | CAGGAAACAGCTATGACCTCAG | 453 |
| | | | | TGTTCCCTTTGTCTCCC | | TCCTTTCCCAAGAGGAT | |
| C1, S subcomponent | C1S | AE111s8 | AE111_X12.f2 | TGTAAAACGACGGCCAGTAGCA | 307 | CAGGAAACAGCTATGACCACCA | 454 |
| | | | | TGTGTTTATTCATCCGG | | CTGTCCCCTTTACAGCT | |
| alanyl aminopeptidase | ANPEP | AE112s57 | AE112_X20.f1 | TGTAAAACGACGGCCAGTGTCC | 308 | CAGGAAACAGCTATGACCCTCC | 455 |
| | | | | CAGACTCCACGGTG | | CCTCAAGGACAAAGTCT | |
| alanyl aminopeptidase | ANPEP | AE112s55 | AE112_X20.f1 | TGTAAAACGACGGCCAGTGTCC | 309 | CAGGAAACAGCTATGACCCTCC | 456 |
| | | | | CAGACTCCACGGTG | | CCTCAAGGACAAAGTCT | |
| alanyl aminopeptidase | ANPEP | AE112s56 | AE112_X20.f1 | TGTAAAACGACGGCCAGTGTCC | 310 | CAGGAAACAGCTATGACCCTCC | 457 |
| | | | | CAGACTCCACGGTG | | CCTCAAGGACAAAGTCT | |
| alanyl aminopeptidase | ANPEP | AE112s58 | AE112_X20.f2 | TGTAAAACGACGGCCAGTCCTG | 311 | CAGGAAACAGCTATGACCGGGA | 458 |
| | | | | GGTCATCAGGAACTAGA | | CTCTAAAGTGGGAGTGG | |
| alanyl aminopeptidase | ANPEP | AE112s44 | AE112_X19 | TGTAAAACGACGGCCAGTAAAG | 312 | CAGGAAACAGCTATGACCGTGA | 459 |
| | | | | TCTGGCTACTCGGCTTC | | CTCCCCCACATCCTAGT | |
| alanyl aminopeptidase | ANPEP | AE112s46 | AE112_X19 | TGTAAAACGACGGCCAGTAAAG | 313 | CAGGAAACAGCTATGACCGTGA | 460 |
| | | | | TCTGGCTACTCGGCTTC | | CTCCCCCACATCCTAGT | |
| alanyl aminopeptidase | ANPEP | AE112s42 | AE112_X19 | TGTAAAACGACGGCCAGTAAAG | 314 | CAGGAAACAGCTATGACCGTGA | 461 |
| | | | | TCTGGCTACTCGGCTTC | | CTCCCCCACATCCTAGT | |
| alanyl aminopeptidase | ANPEP | AE112s43 | AE112_X19 | TGTAAAACGACGGCCAGTAAAG | 315 | CAGGAAACAGCTATGACCGTGA | 462 |
| | | | | TCTGGCTACTCGGCTTC | | CTCCCCCACATCCTAGT | |
| alanyl aminopeptidase | ANPEP | AE112s41 | AE112_X19 | TGTAAAACGACGGCCAGTAAAG | 316 | CAGGAAACAGCTATGACCGTGA | 463 |
| | | | | TCTGGCTACTCGGCTTC | | CTCCCCCACATCCTAGT | |
| alanyl aminopeptidase | ANPEP | AE112s40 | AE112_X18 | TGTAAAACGACGGCCAGTTGGC | 317 | CAGGAAACAGCTATGACCGTTG | 464 |
| | | | | TGATTTTTGTCCACTTC | | AGAGGATGCAGGCATAG | |
| alanyl aminopeptidase | ANPEP | AE112s36 | AE112_X18 | TGTAAAACGACGGCCAGTTGGC | 318 | CAGGAAACAGCTATGACCGTTG | 465 |
| | | | | TGATTTTTGTCCACTTC | | AGAGGATGCAGGCATAG | |
| alanyl aminopeptidase | ANPEP | AE112s38 | AE112_X18 | TGTAAAACGACGGCCAGTTGGC | 319 | CAGGAAACAGCTATGACCGTTG | 466 |
| | | | | TGATTTTTGTCCACTTC | | AGAGGATGCAGGCATAG | |
| alanyl aminopeptidase | ANPEP | AE112s35 | AE112_X18 | TGTAAAACGACGGCCAGTTGGC | 320 | CAGGAAACAGCTATGACCGTTG | 467 |
| | | | | TGATTTTTGTCCACTTC | | AGAGGATGCAGGCATAG | |
| alanyl aminopeptidase | ANPEP | AE112s29 | AE112_X17 | TGTAAAACGACGGCCAGTAATA | 321 | CAGGAAACAGCTATGACCCCTC | 468 |
| | | | | CTGCCTCCACCTCAACA | | CTCTGCCAGTCTTCAT | |
| alanyl aminopeptidase | ANPEP | AE112s33 | AE112_X17 | TGTAAAACGACGGCCAGTAATA | 322 | CAGGAAACAGCTATGACCCCTC | 469 |
| | | | | CTGCCTCCACCTCAACA | | CTCTGCCAGTCTTCAT | |
| alanyl aminopeptidase | ANPEP | AE112s32 | AE112_X17 | TGTAAAACGACGGCCAGTAATA | 323 | CAGGAAACAGCTATGACCCCTC | 470 |
| | | | | CTGCCTCCACCTCAACA | | CTCTGCCAGTCTTCAT | |
| alanyl aminopeptidase | ANPEP | AE112s30 | AE112_X17 | TGTAAAACGACGGCCAGTAATA | 324 | CAGGAAACAGCTATGACCCCTC | 471 |
| | | | | CTGCCTCCACCTCAACA | | CTCTGCCAGTCTTCAT | |
| alanyl aminopeptidase | ANPEP | AE112s26 | AE112_X15 | TGTAAAACGACGGCCAGTCAGA | 325 | CAGGAAACAGCTATGACCCTCT | 472 |
| | | | | GAACACTGCCAGGATGT | | AGACTTCTATGGGCCCC | |
| alanyl aminopeptidase | ANPEP | AE112s22 | AE112_X13 | TGTAAAACGACGGCCAGTCATC | 326 | CAGGAAACAGCTATGACCCTCA | 473 |
| | | | | TGTGAAATGGGTGTGTG | | GCTGCAGAGAGACCACT | |
| alanyl aminopeptidase | ANPEP | AE112s23 | AE112_X13 | TGTAAAACGACGGCCAGTCATC | 327 | CAGGAAACAGCTATGACCCTCA | 474 |
| | | | | TGTGAAATGGGTGTGTG | | GCTGCAGAGAGACCACT | |
| alanyl aminopeptidase | ANPEP | AE112s21 | AE112_X13 | TGTAAAACGACGGCCAGTCATC | 328 | CAGGAAACAGCTATGACCCTCA | 475 |
| | | | | TGTGAAATGGGTGTGTG | | GCTGCAGAGAGACCACT | |
| alanyl aminopeptidase | ANPEP | AE112s17 | AE112_X11 | TGTAAAACGACGGCCAGTCAGC | 329 | CAGGAAACAGCTATGACCGTAG | 476 |
| | | | | AAAGTGGAGATTGGAAC | | ACAGACCCTCCCTCCAG | |
| alanyl aminopeptidase | ANPEP | AE112s18 | AE112_X11 | TGTAAAACGACGGCCAGTCAGC | 330 | CAGGAAACAGCTATGACCGTAG | 477 |
| | | | | AAAGTGGAGATTGGAAC | | ACAGACCCTCCCTCCAG | |
| alanyl aminopeptidase | ANPEP | AE112s19 | AE112_X11 | TGTAAAACGACGGCCAGTCAGC | 331 | CAGGAAACAGCTATGACCGTAG | 478 |
| | | | | AAAGTGGAGATTGGAAC | | ACAGACCCTCCCTCCAG | |
| alanyl aminopeptidase | ANPEP | AE112s71 | AE112_X7 | TGTAAAACGACGGCCAGTCAGA | 332 | CAGGAAACAGCTATGACCGTAC | 479 |
| | | | | CCCTGCCTTCAGTGAG | | CTGGGTGCTGACTATGC | |
| alanyl aminopeptidase | ANPEP | AE112s72 | AE112_X7 | TGTAAAACGACGGCCAGTCAGA | 333 | CAGGAAACAGCTATGACCGTAC | 480 |
| | | | | CCCTGCCTTCAGTGAG | | CTGGGTGCTGACTATGC | |
| alanyl aminopeptidase | ANPEP | AE112s69 | AE112_X6 | TGTAAAACGACGGCCAGTACAC | 334 | CAGGAAACAGCTATGACCGGTC | 481 |
| | | | | ATCATTCAGCACCATGA | | ACGATGGTGTTGAAATC | |
| alanyl aminopeptidase | ANPEP | AE112s68 | AE112_X5 | TGTAAAACGACGGCCAGTGATT | 335 | CAGGAAACAGCTATGACCACTC | 482 |
| | | | | TCAACACCATCGTGACC | | CCAAAATCAGGTGAGTG | |
| alanyl aminopeptidase | ANPEP | AE112s64 | AE112_X4 | TGTAAAACGACGGCCAGTGTCA | 336 | CAGGAAACAGCTATGACCAGGT | 483 |
| | | | | CCAGTCCCCAGTTCTC | | TTAGCTTGATTGGCCTC | |
| alanyl aminopeptidase | ANPEP | AE112s63 | AE112_X4 | TGTAAAACGACGGCCAGTGTCA | 337 | CAGGAAACAGCTATGACCAGGT | 484 |
| | | | | CCAGTCCCCAGTTCTC | | TTAGCTTGATTGGCCTC | |
| alanyl aminopeptidase | ANPEP | AE112s62 | AE112_X4 | TGTAAAACGACGGCCAGTGTCA | 338 | CAGGAAACAGCTATGACCAGGT | 485 |
| | | | | CCAGTCCCCAGTTCTC | | TTAGCTTGATTGGCCTC | |
| alanyl aminopeptidase | ANPEP | AE112s61 | AE112_X4 | TGTAAAACGACGGCCAGTGTCA | 339 | CAGGAAACAGCTATGACCAGGT | 486 |
| | | | | CCAGTCCCCAGTTCTC | | TTAGCTTGATTGGCCTC | |
| alanyl aminopeptidase | ANPEP | AE112s65 | AE112_X4 | TGTAAAACGACGGCCAGTGTCA | 340 | CAGGAAACAGCTATGACCAGGT | 487 |
| | | | | CCAGTCCCCAGTTCTC | | TTAGCTTGATTGGCCTC | |
| alanyl aminopeptidase | ANPEP | AE112s53 | AE112_X2 | TGTAAAACGACGGCCAGTGTGT | 341 | CAGGAAACAGCTATGACCCTGG | 488 |
| | | | | GTGTGAGGACGTACCCT | | TAGCATCCTCAGAGCC | |
| alanyl aminopeptidase | ANPEP | AE112s52 | AE112_X2 | TGTAAAACGACGGCCAGTGTGT | 342 | CAGGAAACAGCTATGACCCTGG | 489 |
| | | | | GTGTGAGGACGTACCCT | | TAGCATCCTCAGAGCC | |
| alanyl aminopeptidase | ANPEP | AE112s13 | AE112_X1.f1 | TGTAAAACGACGGCCAGTCCTG | 343 | CAGGAAACAGCTATGACCTCAT | 490 |
| | | | | CAAGCTAAGTCCTTCCT | | CATCATCCACAGCAAGA | |
| alanyl aminopeptidase | ANPEP | AE112s15 | AE112_X1.f2 | TGTAAAACGACGGCCAGTCTCC | 344 | CAGGAAACAGCTATGACCAATC | 491 |
| | | | | ACCAGCTCAGTCTTGTC | | ATCGCACTGTCAGTGGT | |
| alanyl aminopeptidase | ANPEP | AE112s1 | AE112_X0.f1 | TGTAAAACGACGGCCAGTTCAG | 345 | CAGGAAACAGCTATGACCGTGT | 492 |
| | | | | GCCAGGCAGAGAAC | | TCTTAATAGGAGCCGGG | |
| alanyl aminopeptidase | ANPEP | AE112s5 | AE112_X0.f2 | TGTAAAACGACGGCCAGTGGGA | 346 | CAGGAAACAGCTATGACCCAGC | 493 |
| | | | | ACAGCATCAGAACTGAG | | CGGGAATGATTTTTAA | |
| alanyl aminopeptidase | ANPEP | AE112s4 | AE112_X0.f2 | TGTAAAACGACGGCCAGTGGGA | 347 | CAGGAAACAGCTATGACCCAGC | 494 |
| | | | | ACAGCATCAGAACTGAG | | CGGGAATGATTTTTAA | |
| alanyl aminopeptidase | ANPEP | AE112s3 | AE112_X0.f2 | TGTAAAACGACGGCCAGTGGGA | 348 | CAGGAAACAGCTATGACCCAGC | 495 |
| | | | | ACAGCATCAGAACTGAG | | CGGGAATGATTTTTAA | |
| alanyl aminopeptidase | ANPEP | AE112s2 | AE112_X0.f2 | TGTAAAACGACGGCCAGTGGGA | 349 | CAGGAAACAGCTATGACCCAGC | 496 |
| | | | | ACAGCATCAGAACTGAG | | CGGGAATGATTTTTAA | |
| alanyl aminopeptidase | ANPEP | AE112s9 | AE112_X0.f3 | TGTAAAACGACGGCCAGTTGCC | 350 | CAGGAAACAGCTATGACCAAGG | 497 |
| | | | | TCTCAGGTTTGTTGAAC | | TGTGTATTTTATTTTGTATGGTA | |
| | | | | | | A | |
| alanyl aminopeptidase | ANPEP | AE112s6 | AE112_X0.f3 | TGTAAAACGACGGCCAGTTGCC | 351 | CAGGAAACAGCTATGACCAAGG | 498 |
| | | | | TCTCAGGTTTGTTGAAC | | TGTGTATTTTATTTTGTATGGTA | |
| | | | | | | A | |
| alanyl aminopeptidase | ANPEP | AE112s7 | AE112_X0.f3 | TGTAAAACGACGGCCAGTTGCC | 352 | CAGGAAACAGCTATGACCAAGG | 499 |
| | | | | TCTCAGGTTTGTTGAAC | | TGTGTATTTTATTTTGTATGGTA | |
| | | | | | | A | |
| alanyl aminopeptidase | ANPEP | AE112s11 | AE112_X0.f3 | TGTAAAACGACGGCCAGTTGCC | 353 | CAGGAAACAGCTATGACCAAGG | 500 |
| | | | | TCTCAGGTTTGTTGAAC | | TGTGTATTTTATTTTGTATGGTA | |
| | | | | | | A | |
| alanyl aminopeptidase | ANPEP | AE112s12 | AE112_X0.f3 | TGTAAAACGACGGCCAGITGCC | 354 | CAGGAAACAGCTATGACCAAGG | 501 |
| | | | | TCTCAGGTTTGTTGAAC | | TGTGTATTTTATTTTGTATGGTA | |
| | | | | | | A | |
| meprin A, beta | MEP1B | AE113s4 | AE113_X0.f1 | TGTAAAACGACGGCCAGYI1TAG | 355 | CAGGAAACAGCTATGACCCAAC | 502 |
| | | | | CACAAAATGGGGTGAAG | | CTGTTCTCTCAGTTCCC | |
| meprin A, beta | MEP1B | AE113s3 | AE113_X0.f1 | TGTAAAACGACGGCCAAGTTTAG | 356 | CAGGAAACAGCTATGACCCAAC | 503 |
| | | | | CACAAAATGGGGTGAAG | | CTGTTCTCTCAGTTCCC | |
| meprin A, beta | MEP1B | AE113s2 | AE113_X0.f1 | TGTAAAACGACGGCCAGTTTAG | 357 | CAGGAAACAGCTATGACCCAAC | 504 |
| | | | | CACAAAATGGGGTGAAG | | CTGTTCTCTCAGTTCCC | |
| meprin A, beta | MEP1B | AE113s1 | AE113_X0.f1 | TGTAAAACGACGGCCAGTTTAG | 357 | CAGGAAACAGCTATGACCCAAC | 504 |
| | | | | CACAAAATGGGGTGAAG | | CTGTTCTCTCAGTTCCC | |
| meprin A, beta | MEP1B | AE113s5 | AE113_X0.f2 | TGTAAAACGACGGCCAGTGATT | 359 | CAGGAAACAGCTATGACCTGCC | 506 |
| | | | | ACACATGTGAGCCACCAT | | TGGCTGAGACTTCTTAA | |
| meprin A, beta | MEP1B | AE113s34 | AE113_X3 | TGTAAAACGACGGCCAGTTAGT | 360 | CAGGAAACAGCTATGACCGAAT | 507 |
| | | | | CACCTGAGGCCAAAAGA | | TTGGCTTGCAGTGGTTA | |
| meprin A, beta | MEP1B | AE113s37 | AE113_X5 | TGTAAAACGACGGCCAGTACAC | 361 | CAGGAAACAGCTATGACCCAAA | 508 |
| | | | | ACAATTTGTTTGTGCGA | | GGAGCAAACCAAACAAA | |
| meprin A, beta | MEP1B | AE113s38 | AE113_X6 | TGTAAAACGACGGCCAGTGGCC | 362 | CAGGAAACAGCTATGACCATTG | 509 |
| | | | | AAATAAGGGATCTGAAG | | CTGCAATGTAATGTCCC | |
| meprin A, beta | MEP1B | AE113s39 | AE113_X7 | TGTAAAACGACGGCCAGTAAAG | 363 | CAGGAAACAGCTATGACCCAGT | 510 |
| | | | | GTGGGTAATGAAGTGGG | | TGCCACAGATGACAAGA | |
| meprin A, beta | MEP1B | AE113s41 | AE113_X7 | TGTAAAACGACGGCCAGTAAAG | 364 | CAGGAAACAGCTATGACCCAGT | 511 |
| | | | | GTGGGTAATGAAGTGGG | | TGCCACAGATGACAAGA | |
| meprin A, beta | MEP1B | AE113s40 | AE113_X7 | TGTAAAACGACGGCCAGTAAAG | 365 | CAGGAAACAGCTATGACCCAGT | 512 |
| | | | | GTGGGTAATGAAGTGGG | | TGCCACAGATGACAAGA | |
| meprin A, beta | MEP1B | AE113s43 | AE113_X9 | TGTAAAACGACGGCCAGTCCAA | 366 | CAGGAAACAGCTATGACCATCA | 513 |
| | | | | CCTGGCATTTCTAATACTG | | GGTGCAAGGCCTACTTC | |
| meprin A, beta | MEP1B | AE113s42 | AE113_X9 | TGTAAAACGACGGCCAGTCCAA | 367 | CAGGAAACAGCTATGACCATCA | 514 |
| | | | | CCTGGCATTTCTAATACTG | | GGTGCAAGGCCTACTTC | |
| meprin A, beta | MEP1B | AE113s7 | AE113_X10 | TGTAAAACGACGGCCAGTTGGA | 368 | CAGGAAACAGCTATGACCAGTA | 515 |
| | | | | TGTTACTTATGCCTCGC | | GCCCCTGCCCTATTGTA | |
| meprin A, beta | MEP1B | AE113s6 | AE113_X10 | TGTAAAACGACGGCCAGITGGA | 369 | CAGGAAACAGCTATGACCAGTA | 516 |
| | | | | TGTTACTTATGCCTCGC | | GCCCCTGCCCTATTGTA | |
| meprin A, beta | MEP1B | AE113s9 | AE113_X11.f1 | TGTAAAACGACGGCCAGTGGTT | 370 | CAGGAAACAGCTATGACCATCC | 517 |
| | | | | TTGGATGACCCTTTGTC | | CTGCATTAGTCACATGG | |
| meprin A, beta | MEP1B | AE113s8 | AE113_X11.f1 | TGTAAAACGACGGCCAGTGGTT | 371 | CAGGAAACAGCTATGACCATCC | 518 |
| | | | | TTGGATGACCCTTTGTC | | CTGCATTAGTCACATGG | |
| meprin A, beta | MEP1B | AE113s18 | AE113_X12 | TGTAAAACGACGGCCAGTCCTG | 372 | CAGGAAACAGCTATGACCATAT | 519 |
| | | | | TTCTTTCCTACCACCAA | | TCCTCCCAAACCCCATT | |
| meprin A, beta | MEP1B | AE113s14 | AE113_X12 | TGTAAAACGACGGCCAGTCCTG | 373 | CAGGAAACAGCTATGACCATAT | 520 |
| | | | | TTCTTTCCTACCACCAA | | TCCTCCCAAACCCCATT | |
| meprin A, beta | MEP1B | AE113s16 | AE113_X12 | TGTAAAACGACGGCCAGTCCTG | 374 | CAGGAAACAGCTATGACCATAT | 521 |
| | | | | TTCTTTCCTACCACCAA | | TCCTCCCAAACCCCATT | |
| meprin A, beta | MEP1B | AE113s21 | AE113_X12 | TGTAAAACGACGGCCAGTCCTG | 375 | CAGGAAACAGCTATGACCATAT | 522 |
| | | | | TTCTTTCCTACCACCAA | | TCCTCCCAAACCCCATT | |
| meprin A, beta | MEP1B | AE113s22 | AE113_X13 | TGTAAAACGACGGCCAGTGCAA | 376 | CAGGAAACAGCTATGACCTGAA | 523 |
| | | | | CATTGCAAGATCTCACC | | TGGCTGTGAGATGAGAA | |
| meprin A, beta | MEP1B | AE113s24 | AE113_X14 | TGTAAAACGACGGCCAGTGGTG | 377 | CAGGAAACAGCTATGACCAAAA | 524 |
| | | | | ATCCACATCTTTAACTGTGA | | ATGTCCTAGTTCCTCTCCA | |
| meprin A, beta | MEP1B | AE113s25 | AE113_X14 | TGTAAAACGACGGCCAGTGGTG | 378 | CAGGAAACAGCTATGACCAAAA | 525 |
| | | | | ATCCACATCTTTAACTGTGA | | ATGTCCTAGTTCCTCTCCA | |
| meprin A, beta | MEP1B | AE113s26 | AE113_X14 | TGTAAAACGACGGCCAGTGGTG | 379 | CAGGAAACAGCTATGACCAAAA | 526 |
| | | | | ATCCACATCTTTAACTGTGA | | ATGTCCTAGTTCCTCTCCA | |
| meprin A, beta | MEP1B | AE113s31 | AE113_X15 | TGTAAAACGACGGCCAGTTGCT | 380 | CAGGAAACAGCTATGACCGAAA | 527 |
| | | | | GATGGGGACTTCATTTA | | AGCCATCTCTAACTGTTGC | |
| meprin A, beta | MEP1B | AE113s30 | AE113_X15 | TGTAAAACGACGGCCAGTTGCT | 381 | CAGGAAACAGCTATGACCGAAA | 528 |
| | | | | GATGGGGACTTCATTTA | | AGCCATCTCTAACTGTTGC | |
| Aminopeptidase P-like | XPNPEPL | AE114s30 | AE114_X19 | TGTAAAACGACGGCCAGTAAGG | 382 | CAGGAAACAGCTATGACCGTAT | 529 |
| | | | | AAAGGGGAAAGATGTCA | | TTCCGGGGAAACAGAAC | |
| Aminopeptidase P-like | XPNPEPL | AE114s31 | AE114_X19 | TGTAAAACGACGGCCAGTAAGG | 383 | CAGGAAACAGCTATGACCGTAT | 530 |
| | | | | AAAGGGGAAAGATGTCA | | TTCCGGGGAAACAGAAC | |
| Aminopeptidase P-like | XPNPEPL | AE114s29 | AE114_X16 | TGTAAAACGACGGCCAGTGGAA | 384 | CAGGAAACAGCTATGACCCGTT | 531 |
| | | | | GGGAAAAGTATTGCCAA | | CTGGAGCCACACATAAT | |
| Aminopeptidase P-like | XPNPEPL | AE114s28 | AE114_X16 | TGTAAAACGACGGCCAGTGGAA | 385 | CAGGAAACAGCTATGACCCGTT | 532 |
| | | | | GGGAAAAGTATTGCCAA | | CTGGAGCCACACATAAT | |
| Aminopeptidase P-like | XPNPEPL | AE114s26 | AE114_X15 | TGTAAAACGACGGCCAGTTATG | 386 | CAGGAAACAGCTATGACCTTAG | 533 |
| | | | | GAGACTCACCCCATGAG | | CAGGCAGGATTTTCTGA | |
| Aminopeptidase P-like | XPNPEPL | AE114s25 | AE114_X14 | TGTAAAACGACGGCCAGTATAA | 387 | CAGGAAACAGCTATGACCTCTC | 534 |
| | | | | GCCTGACTCCACAGCAA | | ACTTGTCCACATGCTTG | |
| Aminopeptidase P-like | XPNPEPL | AE114s24 | AE114_X14 | TGTAAAACGACGGCCAGTATAA | 388 | CAGGAAACAGCTATGACCTCTC | 535 |
| | | | | GCCTGACTCCACAGCAA | | ACTTGTCCACATGCTTG | |
| Aminopeptidase P-like | XPNPEPL | AE114s23 | AE114_X13 | TGTAAAACGACGGCCAGTAAAC | 389 | CAGGAAACAGCTATGACCAGCT | 536 |
| | | | | TTCCAGCCAAGGACTGT | | CTTGTGTGTCCTCCTCC | |
| Aminopeptidase P-like | XPNPEPL | AE114s22 | AE114_X13 | TGTAAAACGACGGCCAGTAAAC | 390 | CAGGAAACAGCTATGACCAGCT | 537 |
| | | | | TTCCAGCCAAGGACTGT | | CTTGTGTGTCCTCCTCC | |
| Aminopeptidase P-like | XPNPEPL | AE114s18 | AE114_X12 | TGTAAAACGACGGCCAGTTAAG | 391 | CAGGAAACAGCTATGACCTTGCT | 538 |
| | | | | AAGAGGCTCCCAAAAGG | | AGTTGAATGAGGCTGG | |
| Aminopeptidase P-like | XPNPEPL | AE114s14 | AE114_X11 | TGTAAAACGACGGCCAGTAAAT | 392 | CAGGAAACAGCTATGACCGTGA | 539 |
| | | | | TGTTGGGAAGCTCAGGT | | GAGCCTTTGGGAGTTCT | |
| Aminopeptidase P-like | XPNPEPL | AE114s13 | AE114_X11 | TGTAAAACGACGGCCAGTAAAT | 393 | CAGGAAACAGCTATGACCGTGA | 540 |
| | | | | TGTTGGGAAGCTCAGGT | | GAGCCTTTGGGAGTTCT | |
| Aminopeptidase P-like | XPNPEPL | AE114s16 | AE114_X11 | TGTAAAACGACGGCCAGTAAAT | 394 | CAGGAAACAGCTATGACCGTGA | 541 |
| | | | | TGTTGGGAAGCTCAGGT | | GAGCCTTTGGGAGTTCT | |
| Aminopeptidase P-like | XPNPEPL | AE114s12 | AE114_X10 | TGTAAAACGACGGCCAGTAACC | 395 | CAGGAAACAGCTATGACCTGCA | 542 |
| | | | | CAAAAAGCATCCACTCT | | GGTACAGCCTTCTCAGT | |
| Aminopeptidase P-like | XPNPEPL | AE114s45 | AE114_X9 | TGTAAAACGACGGCCAGTCAAT | 396 | CAGGAAACAGCTATGACCAAAA | 543 |
| | | | | AACTCCTCCTGGAAGGC | | GGTTGTTGCCTTGGTCT | |
| Aminopeptidase P-like | XPNPEPL | AE114s44 | AE114_X9 | TGTAAAACGACGGCCAGTCAAT | 397 | CAGGAAACAGCTATGACCAAAA | 544 |
| | | | | AACTCCTCCTGGAAGGC | | GGTTGTTGCCTTGGTCT | |
| Aminopeptidase P-like | XPNPEPL | AE114s43 | AE114_X8 | TGTAAAACGACGGCCAGTCTCA | 398 | CAGGAAACAGCTATGACCAGAG | 545 |
| | | | | TCCATGGAGACCACAGT | | AAAAGGGGCTTGGTTTT | |
| Aminopeptidase P-like | XPNPEPL | AE114s42 | AE114_X7 | TGTAAAACGACGGCCAGTGGGA | 399 | CAGGAAACAGCTATGACCACTC | 546 |
| | | | | AAATGTTAAAAGGGCAA | | CAAGCTGAAGCTCTTCC | |
| Aminopeptidase P-like | XPNPEPL | AE114s41 | AE114_X6 | TGTAAAACGACGGCCAGTCTTCT | 400 | CAGGAAACAGCTATGACCCCTG | 547 |
| | | | | TATTCCCTGGCCACTC | | CCTGTTTAAGTAGCGTG | |
| Aminopeptidase P-like | XPNPEPL | AE114s35 | AE114_X5 | TGTAAAACGACGGCCAGTGTAG | 401 | CAGGAAACAGCTATGACCGGAC | 548 |
| | | | | GGCAAAATCCTCCTGTC | | CACCTTTCTGAAGGTTC | |
| Aminopeptidase P-like | XPNPEPL | AE114s34 | AE114_X5 | TGTAAAACGACGGCCAGTGTAG | 402 | CAGGAAACAGCTATGACCGGAC | 549 |
| | | | | GGCAAAATCCTCCTGTC | | CACCTTTCTGAAGGTTC | |
| Aminopeptidase P-like | XPNPEPL | AE114s36 | AE114_X5 | TGTAAAACGACGGCCAGTGTAG | 403 | CAGGAAACAGCTATGACCGGAC | 550 |
| | | | | GGCAAAATCCTCCTGTC | | CACCTTTCTGAAGGTTC | |
| Aminopeptidase P-like | XPNPEPL | AE114s38 | AE114_X5 | TGTAAAACGACGGCCAGTGTAG | 404 | CAGGAAACAGCTATGACCGGAC | 551 |
| | | | | GGCAAAATCCTCCTGTC | | CACCTTTCTGAAGGTTC | |
| Aminopeptidase P-like | XPNPEPL | AE114s33 | AE114_X3 | TGTAAAACGACGGCCAGTTCCTT | 405 | CAGGAAACAGCTATGACCTGTC | 552 |
| | | | | GGATTTTCTCAAAAGGGT | | ATTTTGTGCAGCTTCTG | |
| Aminopeptidase P-like | XPNPEPL | AE114s32 | AE114_X2 | TGTAAAACGACGGCCAGTGTCA | 406 | CAGGAAACAGCTATGACCTCAA | 553 |
| | | | | ATGTGGAGCATCTGGTT | | TCTTCTGCAGTGTGTGC | |
| Aminopeptidase P-like | XPNPEPL | AE114s5 | AE114_X0.f1 | TGTAAAACGACGGCCAGTATCG | 407 | CAGGAAACAGCTATGACCTGAC | 554 |
| | | | | GTTCGGTCACATACTCA | | CATACACTTCTCCTGCTT | |
| Aminopeptidase P-like | XPNPEPL | AE114s3 | AE114_X0.f1 | TGTAAAACGACGGCCAGTATCG | 408 | CAGGAAACAGCTATGACCTGAC | 555 |
| | | | | GTTCGGTCACATACTCA | | CATACACTTCTCCTGCTT | |
| Aminopeptidase P-like | XPNPEPL | AE114s7 | AE114_X0.f2 | TGTAAAACGACGGCCAGTTGAC | 409 | CAGGAAACAGCTATGACCCTCCT | 556 |
| | | | | TGGTTACCTCTTTCACTCC | | GAGTGCAAGTGATTCC | |
| Aminopeptidase P-like | XPNPEPL | AE114s6 | AE114_X0.f2 | TGTAAAACGACGGCCAGTTGAC | 410 | CAGGAAACAGCTATGACCCTCCT | 557 |
| | | | | TGGTTACCTCTTTCACTCC | | GAGTGCAAGTGATTCC | |
| Aminopeptidase P-like | XPNPEPL | AE114s9 | AE114_X0.f3 | TGTAAAACGACGGCCAGTATCA | 411 | CAGGAAACAGCTATGACCACAG | 558 |
| | | | | GCCAGGTGTGGTGG | | CTGGTTGGTAGCAGGTA | |
| Tissue kallikrein | KLK1 | AE115s13 | AE115_X4 | TGTAAAACGACGGCCAGTGTGA | 412 | CAGGAAACAGCTATGACCAATT | 559 |
| | | | | AGCAGATGCCTGGTTAG | | GTATGTGGGGGCAGACT | |
| Tissue kallikrein | KLK1 | AE115s12 | AE115_X4 | TGTAAAACGACGGCCAGTGTGA | 413 | CAGGAAACAGCTATGACCAATT | 560 |
| | | | | AGCAGATGCCTGGTTAG | | GTATGTGGGGGCAGACT | |
| Tissue kallikrein | KLK1 | AE115s11 | AE115_X4 | TGTAAAACGACGGCCAGTGTGA | 414 | CAGGAAACAGCTATGACCAATT | 561 |
| | | | | AGCAGATGCCTGGTTAG | | GTATGTGGGGGCAGACT | |
| Tissue kallikrein | KLK1 | AE115s14 | AE115_X4 | TGTAAAACGACGGCCAGTGTGA | 415 | CAGGAAACAGCTATGACCAATT | 562 |
| | | | | AGCAGATGCCTGGTTAG | | GTATGTGGGGGCAGACT | |
| Tissue kallikrein | KLK1 | AE115s1 | AE115_X0.f2 | TGTAAAACGACGGCCAGTGGGA | 416 | CAGGAAACAGCTATGACCAGAT | 563 |
| | | | | AGCAGAAACAGGAAGAC | | CTCTGGGACTTTGGAGG | |
| Tissue kallikrein | KLK1 | AE115s4 | AE115_X0.f3 | TGTAAAACGACGGCCAGTGGGT | 417 | CAGGAAACAGCTATGACCATTT | 564 |
| | | | | CTCCCAGGGTAAGTTCT | | ACTTGTGGGCTTCCTGG | |
| Tissue kallikrein | KLK1 | AE115s3 | AE115_X0.f3 | TGTAAAACGACGGCCAGTGGGT | 418 | CAGGAAACAGCTATGACCATTT | 565 |
| | | | | CTCCCAGGGTAAGTTCT | | ACTTGTGGGCTTCCTGG | |
| Aminopeptidase P | XPNPEP2 | AE116s1 | AE116_X0.f1 | TGTAAAACGACGGCCAGTCTCC | 419 | CAGGAAACAGCTATGACCGCAT | 566 |
| (membrane bound) | | | | AGGCTAGGTGAGCATC | | TTTCTGAACCTGCACTC | |
| Aminopeptidase P | XPNPEP2 | AE116s2 | AE116_X0.f3 | TGTAAAACGACGGCCAGTAGGA | 420 | CAGGAAACAGCTATGACCGTCTT | 567 |
| (membrane bound) | | | | GGATGAGGAGGGAAAA | | TTGTTTTGGAGGGCTC | |
| Aminopeptidase P | XPNPEP2 | AE116s4 | AE116_X1.f1 | TGTAAAACGACGGCCAGTAAAA | 421 | CAGGAAACAGCTATGACCATGC | 568 |
| (membrane bound) | | | | GCAGGCTGATTGAGACC | | ACATACCACAGAGGAGG | |
| Aminopeptidase P | XPNPEP2 | AE116s3 | AE116_X1.f1 | TGTAAAACGACGGCCAGTAAAA | 422 | CAGGAAACAGCTATGACCATGC | 569 |
| (membrane bound) | | | | GCAGGCTGATTGAGACC | | ACATACCACAGAGGAGG | |
| Aminopeptidase P | XPNPEP2 | AE116s29 | AE116_X4 | TGTAAAACGACGGCCAGTCTGC | 423 | CAGGAAACAGCTATGACCCCAG | 570 |
| (membrane bound) | | | | AAAAGATACGGTTGCTC | | ATGCTGAGACACTAGCC | |
| Aminopeptidase P | XPNPEP2 | AE116s30 | AE116_X5 | TGTAAAACGACGGCCAG3TGAT | 424 | CAGGAAACAGCTATGACCTGCA | 571 |
| (membrane bound) | | | | TCAGGACACCTTTCTGC | | CGCTCTCACCTATACCT | |
| Aminopeptidase P | XPNPEP2 | AE116s32 | AE116_X6 | TGTAAAACGACGGCCAGTAGGA | 425 | CAGGAAACAGCTATGACCCCTC | 572 |
| (membrane bound) | | | | AATTTGAGGCCATCACT | | CTTCTACCAAGGTCCAT | |
| Aminopeptidase P | XPNPEP2 | AE116s33 | AE116_X7 | TGTAAAACGACGGCCAGTGCAA | 426 | CAGGAAACAGCTATGACCTAAA | 573 |
| (membrane bound) | | | | AGGGAACCAGGACTAAC | | CAAGCATCCCAGGTGAC | |
| Aminopeptidase P | XPNPEP2 | AE116s7 | AE116_X10 | TGTAAAACGACGGCCAGTCTTC | 427 | CAGGAAACAGCTATGACCAGGG | 574 |
| (membrane bound) | | | | CTTTGACCTCCAGGAAC | | GCTTCACCTCACTTTAG | |
| AminopeptidaseP | XPNPEP2 | AE116s18 | AE116_X21.f1 | TGTAAAACGACGGCCAGTGGAC | 428 | CAGGAAACAGCTATGACCTGCA | 575 |
| (membrane bound) | | | | TATGGTGACAGCTGGAG | | AAGGTTTCTAGGCAATG | |
| Aminopeptidase P | XPNPEP2 | AE116s28 | AE116_X21.f4 | TGTAAAACGACGGCCAGTAGCC | 429 | CAGGAAACAGCTATGACCCCAC | 576 |
| (membrane bound) | | | | ACAGCTACAATGCTGTT | | CTCACCCTCTCTTCTTC | |
| Aminopeptidase P | XPNPEP2 | AE116s21 | AE116_X21.f3 | TGTAAAACGACGGCCAGTAGAG | 430 | CAGGAAACAGCTATGACCTTGT | 577 |
| (membrane bound) | | | | CCCAAACCTATCACCAC | | GTGTCCTTAGGCAAAGC | |
| Soluble guanylate | GUCY1A2 | AE117s11 | AE117_X6 | TGTAAAACGACGGCCAGTGATG | 431 | CAGGAAACAGCTATGACCTCCTC | 578 |
| cyclase 1, alpha-2 | | | | TTTTGCCGACATGTTTT | | CTTAAGCACCCAACTT | |
| subunit | | | | | | | |
| Soluble guanylate | GUCY1A2 | AE117s13 | AE117_X7 | TGTAAAACGACGGCCAGTTTCCT | 432 | CAGGAAACAGCTATGACCGGGA | 579 |
| cyclase 1, alpha-2 | | | | GTCTTTGAGGAGCTCA | | CTCACAGATGACAGCAT | |
| subunit | | | | | | | |
| Soluble guanylate | GUCY1A2 | AE117s7 | AE117_X3 | TGTAAAACGACGGCCAGTAACA | 433 | CAGGAAACAGCTATGACCTGAA | 580 |
| cyclase 1, alpha-2 | | | | CCCATCACTTCACAAGG | | AGCATAGACTGCGTGTG | |
| subunit | | | | | | | |
| Soluble guanylate | GUCY1A2 | AE117s6 | AE117_X3 | TGTAAAACGACGGCCAGTAACA | 434 | CAGGAAACAGCTATGACCTGAA | 581 |
| cyclase 1, alpha-2 | | | | CCCATCACTTCACAAGG | | AGCATAGACTGCGTGTG | |
| subunit | | | | | | | |
| Soluble guanylate | GUCY1A2 | AE117s10 | AE117_X3 | TGTAAAACGACGGCCAGTAACA | 435 | CAGGAAACAGCTATGACCTGAA | 582 |
| cyclase 1, alpha-2 | | | | CCCATCACTTCACAAGG | | AGCATAGACTGCGTGTG | |
| subunit | | | | | | | |
| Soluble guanylate | GUCY1A2 | AE117s9 | AE117_X3 | TGTAAAACGACGGCCAGTAACA | 436 | CAGGAAACAGCTATGACCTGAA | 583 |
| cyclase 1, alpha-2 | | | | CCCATCACTTCACAAGG | | AGCATAGACTGCGTGTG | |
| subunit | | | | | | | |
| Soluble guanylate | GUCY1A2 | AE117s14 | AE117_X8.f1 | TGTAAAACGACGGCCAGTAGGC | 437 | CAGGAAACAGCTATGACCTTTTG | 584 |
| cyclase 1, alpha-2 | | | | TTCCTGAGCCTTTAAAA | | CTGCTTGTTCTCAACA | |
| subunit | | | | | | | |
| Soluble guanylate | GUCY1A2 | AE117s15 | AE117_X8.f2 | TGTAAAACGACGGCCAGTCCAG | 438 | CAGGAAACAGCTATGACCTTGG | 585 |
| cyclase 1, alpha-2 | | | | AACATGGGTCACCAA | | GAAGTITTACCACACTGC | |
| subunit | | | | | | | |
| Soluble guanylate | GUCY1A2 | AE117s1 | AE117_X0.f1 | TGTAAAACGACGGCCAGTGGAA | 439 | CAGGAAACAGCTATGACCGACT | 586 |
| cyclase 1, alpba-2 | | | | GGACTGGTTTGCAAAGA | | TGTGTTCCCCGCCT | |
| subunit | | | | | | | |
| Soluble guanylate | GUCY1A2 | AE117s2 | AE117_X0.f1 | TGTAAAACGACGGCCAGTGGAA | 440 | CAGGAAACAGCTATGACCGACT | 587 |
| cyclase 1, alpba-2 | | | | GGACTGGTTTGCAAAGA | | TGTGTTCCCCGCCT | |
| subunit | | | | | | | |
| Soluble guanylate | GUCY1A2 | AE117s3 | AE117_X0.f1 | TGTAAAACGACGGCCAGTGGAA | 441 | CAGGAAACAGCTATGACCGACT | 588 |
| cyclase 1, alpha-2 | | | | GGACTGGTTTGCAAAGA | | TGTGTTCCCCGCCT | |
| subunit | | | | | | | |

TABLE V

| | | | | | | REVERSE_SEQUENCING |
|---|---|---|---|---|---|---|
| GENE_{—} | | | | FORWARD_SEQUENCING_PRIMER | | _PRIMER |
| DESCRIPTION | HGNC_ID | SNP_ID | FORWARD_SEQUENCING_PRIMER | (SEQ ID NO:) | REVERSE_SEQUENCING_PRIMER | (SEQ ID NO:) |
| C1, S subcomponent | C1S | AE111s1 | TGTAAAACGACGGCCAGT | 589 | CAGGAAACAGCTATGACC | 736 |
| C1, S subcomponent | C1S | AE111s2 | TGTAAAACGACGGCCAGT | 590 | CAGGAAACAGCTATGACC | 737 |
| C1, S subcomponent | C1S | AE111s13 | TGTAAAACGACGGCCAGT | 591 | CAGGAAACAGCTATGACC | 738 |
| C1, S subcomponent | C1S | AE111s15 | TGTAAAACGACGGCCAGT | 592 | CAGGAAACAGCTATGACC | 739 |
| C1, S subcomponent | C1S | AE111s17 | TGTAAAACGACGGCCAGT | 593 | CAGGAAACAGCTATGACC | 740 |
| C1, S subcomponent | C1S | AE111s19 | TGTAAAACGACGGCCAGT | 594 | CAGGAAACAGCTATGACC | 741 |
| C1, S subcomponent | C1S | AE111s18 | TGTAAAACGACGGCCAGT | 595 | CAGGAAACAGCTATGACC | 742 |
| C1, S subcomponent | C1S | AE111s20 | TGTAAAACGACGGCCAGT | 596 | CAGGAAACAGCTATGACC | 743 |
| C1, S subcomponent | C1S | AE111s21 | TGTAAAACGACGGCCAGT | 597 | CAGGAAACAGCTATGACC | 744 |
| C1, S subcomponent | C1S | AE111s22 | TGTAAAACGACGGCCAGT | 598 | CAGGAAACAGCTATGACC | 745 |
| C1, S subcomponent | C1S | AE111s5 | TGTAAAACGACGGCCAGT | 599 | CAGGAAACAGCTATGACC | 746 |
| C1, S subcomponent | C1S | AE111s6 | TGTAAAACGACGGCCAGT | 600 | CAGGAAACAGCTATGACC | 747 |
| C1, S subcomponent | C1S | AE111s8 | TGTAAAACGACGGCCAGT | 601 | CAGGAAACAGCTATGACC | 748 |
| alanyl aminopeptidase | ANPEP | AE112s57 | TGTAAAACGACGGCCAGT | 602 | CAGGAAACAGCTATGACC | 749 |
| alanyl aminopeptidase | ANPEP | AE112s55 | TGTAAAACGACGGCCAGT | 603 | CAGGAAACAGCTATGACC | 750 |
| alanyl aminopeptidase | ANPEP | AE112s56 | TGTAAAACGACGGCCAGT | 604 | CAGGAAACAGCTATGACC | 751 |
| alanyl aminopeptidase | ANPEP | AE112s58 | TGTAAAACGACGGCCAGT | 605 | CAGGAAACAGCTATGACC | 752 |
| alanyl aminopeptidase | ANPEP | AE112s44 | TGTAAAACGACGGCCAGT | 606 | CAGGAAACAGCTATGACC | 753 |
| alanyl aminopeptidase | ANPEP | AE112s46 | TGTAAAACGACGGCCAGT | 607 | CAGGAAACAGCTATGACC | 754 |
| alanyl aminopeptidase | ANPEP | AE112s42 | TGTAAAACGACGGCCAGT | 608 | CAGGAAACAGCTATGACC | 755 |
| alanyl aminopeptidase | ANPEP | AE112s43 | TGTAAAACGACGGCCAGT | 609 | CAGGAAACAGCTATGACC | 756 |
| alanyl aminopeptidase | ANPEP | AE112s41 | TGTAAAACGACGGCCAGT | 610 | CAGGAAACAGCTATGACC | 757 |
| alanyl aminopeptidase | ANPEP | AE112s40 | TGTAAAACGACGGCCAGT | 611 | CAGGAAACAGCTATGACC | 758 |
| alanyl aminopeptidase | ANPEP | AE112s36 | TGTAAAACGACGGCCAGT | 612 | CAGGAAACAGCTATGACC | 759 |
| alanyl aminopeptidase | ANPEP | AE112s38 | TGTAAAACGACGGCCAGT | 613 | CAGGAAACAGCTATGACC | 760 |
| alanyl aminopeptidase | ANPEP | AE112s35 | TGTAAAACGACGGCCAGT | 614 | CAGGAAACAGCTATGACC | 761 |
| alanyl aminopeptidase | ANPEP | AE112s29 | TGTAAAACGACGGCCAGT | 615 | CAGGAAACAGCTATGACC | 762 |
| alanyl aminopeptidase | ANPEP | AE112s33 | TGTAAAACGACGGCCAGT | 616 | CAGGAAACAGCTATGACC | 763 |
| alanyl aminopeptidase | ANPEP | AE112s32 | TGTAAAACGACGGCCAGT | 617 | CAGGAAACAGCTATGACC | 764 |
| alanyl aminopeptidase | ANPEP | AE112s30 | TGTAAAACGACGGCCAGT | 618 | CAGGAAACAGCTATGACC | 765 |
| alanyl aminopeptidase | ANPEP | AE112s26 | TGTAAAACGACGGCCAGT | 619 | CAGGAAACAGCTATGACC | 766 |
| alanyl aminopeptidase | ANPEP | AE112s22 | TGTAAAACGACGGCCAGT | 620 | CAGGAAACAGCTATGACC | 767 |
| alanyl aminopeptidase | ANPEP | AE112s23 | TGTAAAACGACGGCCAGT | 621 | CAGGAAACAGCTATGACC | 768 |
| alanyl aminopeptidase | ANPEP | AE112s21 | TGTAAAACGACGGCCAGT | 622 | CAGGAAACAGCTATGACC | 769 |
| alanyl aminopeptidase | ANPEP | AE112s17 | TGTAAAACGACGGCCAGT | 623 | CAGGAAACAGCTATGACC | 770 |
| alanyl aminopeptidase | ANPEP | AE112s18 | TGTAAAACGACGGCCAGT | 624 | CAGGAAACAGCTATGACC | 771 |
| alanyl aminopeptidase | ANPEP | AE112s19 | TGTAAAACGACGGCCAGT | 625 | CAGGAAACAGCTATGACC | 772 |
| alanyl aminopeptidase | ANPEP | AE112s71 | TGTAAAACGACGGCCAGT | 626 | CAGGAAACAGCTATGACC | 773 |
| alanyl aminopeptidase | ANPEP | AE112s72 | TGTAAAACGACGGCCAGT | 627 | CAGGAAACAGCTATGACC | 774 |
| alanyl aminopeptidase | ANPEP | AE112s69 | TGTAAAACGACGGCCAGT | 628 | CAGGAAACAGCTATGACC | 775 |
| alanyl aminopeptidase | ANPEP | AE112s68 | TGTAAAACGACGGCCAGT | 629 | CAGGAAACAGCTATGACC | 776 |
| alanyl aminopeptidase | ANPEP | AE112s64 | TGTAAAACGACGGCCAGT | 630 | CAGGAAACAGCTATGACC | 777 |
| alanyl aminopeptidase | ANPEP | AE112s63 | TGTAAAACGACGGCCAGT | 631 | CAGGAAACAGCTATGACC | 778 |
| alanyl aminopeptidase | ANPEP | AE112s62 | TGTAAAACGACGGCCAGT | 632 | CAGGAAACAGCTATGACC | 779 |
| alanyl aminopeptidase | ANPEP | AE112s61 | TGTAAAACGACGGCCAGT | 633 | CAGGAAACAGCTATGACC | 780 |
| alanyl aminopeptidase | ANPEP | AE112s65 | TGTAAAACGACGGCCAGT | 634 | CAGGAAACAGCTATGACC | 781 |
| alanyl aminopeptidase | ANPEP | AE112s53 | TGTAAAACGACGGCCAGT | 635 | CAGGAAACAGCTATGACC | 782 |
| alanyl aminopeptidase | ANPEP | AE112s52 | TGTAAAACGACGGCCAGT | 636 | CAGGAAACAGCTATGACC | 783 |
| alanyl aminopeptidase | ANPEP | AE112s13 | TGTAAAACGACGGCCAGT | 637 | CAGGAAACAGCTATGACC | 784 |
| alanyl aminopeptidase | ANPEP | AE112s15 | TGTAAAACGACGGCCAGT | 638 | CAGGAAACAGCTATQACC | 785 |
| alanyl aminopeptidase | ANPEP | AE112s1 | TGTAAAACGACGGCCAGT | 639 | CAGGAAACAGCTATGACC | 786 |
| alanyl aminopeptidase | ANPEP | AE112s5 | TGTAAAACGACGGCCAGT | 640 | CAGGAAACAGCTATGACC | 787 |
| alanyl aminopeptidase | ANPEP | AE112s4 | TGTAAAACGACGGCCAGT | 641 | CAGGAAACAGCTATGACC | 788 |
| alanyl aminopeptidase | ANPEP | AE112s3 | TGTAAAACGACGGCCAGT | 642 | CAGGAAACAGCTATGACC | 789 |
| alanyl aminopeptidase | ANPEP | AE112s2 | TGTAAAACGACGGCCAGT | 643 | CAGGAAACAGCTATGACC | 790 |
| alanyl aminopeptidase | ANPEP | AE112s9 | TGTAAAACGACGGCCAGT | 644 | CAGGAAACAGCTATGACC | 791 |
| alanyl aminopeptidase | ANPEP | AE112s6 | TGTAAAACGACGGCCAGT | 645 | CAGGAAACAGCTATGACC | 792 |
| alanyl aminopeptidase | ANPEP | AE112s7 | TGTAAAACGACGGCCAGT | 646 | CAGGAAACAGCTATGACC | 793 |
| alanyl aminopeptidase | ANPEP | AE112s11 | TGTAAAACGACGGCCAGT | 647 | CAGGAAACAGCTATGACC | 794 |
| alanyl aminopeptidase | ANPEP | AE112s12 | TGTAAAACGACGGCCAGT | 648 | CAGGAAACAGCTATGACC | 795 |
| meprin A, beta | MEP1B | AE113s4 | TGTAAAACGACGGCCAGT | 649 | CAGGAAACAGCTATGACC | 796 |
| meprin A, beta | MEP1B | AE113s3 | TGTAAAACGACGGCCAGT | 650 | CAGGAAACAGCTATGACC | 797 |
| meprin A, beta | MEP1B | AE113s2 | TGTAAAACGACGGCCAGT | 651 | CAGGAAACAGCTATGACC | 798 |
| meprin A, beta | MEP1B | AE113s1 | TGTAAAACGACGGCCAGT | 652 | CAGGAAACAGCTATGACC | 799 |
| meprin A, beta | MEP1B | AE113s5 | TGTAAAACGACGGCCAGT | 653 | CAGGAAACAGCTATGACC | 800 |
| meprin A, beta | MEP1B | AE113s34 | TGTAAAACGACGGCCAGT | 654 | CAGGAAACAGCTATGACC | 801 |
| meprin A, beta | MEP1B | AE113s37 | TGTAAAACGACGGCCAGT | 655 | CAGGAAACAGCTATGACC | 802 |
| meprin A, beta | MEP1B | AE113s38 | TGTAAAACGACGGCCAGT | 656 | CAGGAAACAGCTATGACC | 803 |
| meprin A, beta | MEP1B | AE113s39 | TGTAAAACGACGGCCAGT | 657 | CAGGAAACAGCTATGACC | 804 |
| meprin A, beta | MEP1B | AE113s41 | TGTAAAACGACGGCCAGT | 658 | CAGGAAACAGCTATGACC | 805 |
| meprin A, beta | MEP1B | AE113s40 | TGTAAAACGACGGCCAGT | 659 | CAGGAAACAGCTATGACC | 806 |
| meprin A, beta | MEP1B | AE113s43 | TGTAAAACGACGGCCAGT | 660 | CAGGAAACAGCTATGACC | 807 |
| meprin A, beta | MEP1B | AE113s42 | TGTAAAACGACGGCCAGT | 661 | CAGGAAACAGCTATGACC | 808 |
| meprin A, beta | MEP1B | AE113s7 | TGTAAAACGACGGCCAGT | 662 | CAGGAAACAGCTATGACC | 809 |
| meprin A, beta | MEP1B | AE113s6 | TGTAAAACGACGGCCAGT | 663 | CAGGAAACAGCTATGACC | 810 |
| meprin A, beta | MEP1B | AE113s9 | TGTAAAACGACGGCCAGT | 664 | CAGGAAACAGCTATGACC | 811 |
| meprin A, beta | MEP1B | AE113s8 | TGTAAAACGACGGCCAGT | 665 | CAGGAAACAGCTATGACC | 812 |
| meprin A, beta | MEP1B | AE113s18 | TGTAAAACGACGGCCAGT | 666 | CAGGAAACAGCTATGACC | 813 |
| meprin A, beta | MEP1B | AE113s14 | TGTAAAACGACGGCCAGT | 667 | CAGGAAACAGCTATGACC | 814 |
| meprin A, beta | MEP1B | AE113s16 | TGTAAAACGACGGCCAGT | 668 | CAGGAAACAGCTATGACC | 815 |
| meprin A, beta | MEP1B | AE113s21 | TGTAAAACGACGGCCAGT | 669 | CAGGAAACAGCTATGACC | 816 |
| meprin A, beta | MEP1B | AE113s22 | TGTAAAACGACGGCCAGT | 670 | CAGGAAACAGCTATGACC | 817 |
| meprin A, beta | MEP1B | AE113s24 | TGTAAAACGACGGCCAGT | 671 | CAGGAAACAGCTATGACC | 818 |
| meprin A, beta | MEP1B | AE113s25 | TGTAAAACGACGGCCAGT | 672 | CAGGAAACAGCTATGACC | 819 |
| meprin A, beta | MEP1B | AE113s26 | TGTAAAACGACGGCCAGT | 673 | CAGGAAACAGCTATGACC | 820 |
| meprin A, beta | MEP1B | AE113s31 | TGTAAAACGACGGCCAGT | 674 | CAGGAAACAGCTATGACC | 821 |
| meprin A, beta | MEP1B | AE113s30 | TGTAAAACGACGGCCAGT | 675 | CAGGAAACAGCTATGACC | 822 |
| Aminopeptidase P-like | XPNPEPL | AE114s30 | TGTAAAACGACGGCCAGT | 676 | CAGGAAACAGCTATGACC | 823 |
| Aminopeptidase P-like | XPNPEPL | AE114s31 | TGTAAAACGACGGCCAGT | 677 | CAGGAAACAGCTATGACC | 824 |
| Aminopeptidase P-like | XPNPEPL | AE114s29 | TGTAAAACGACGGCCAGT | 678 | CAGGAAACAGCTATGACC | 825 |
| Aminopeptidase P-like | XPNPEPL | AE114s28 | TGTAAAACGACGGCCAGT | 679 | CAGGAAACAGCTATGACC | 826 |
| Aminopeptidase P-like | XPNPEPL | AE114s26 | TGTAAAACGACGGCCAGT | 680 | CAGGAAACAGCTATGACC | 827 |
| Aminopeptidase P-like | XPNPEPL | AE114s25 | TGTAAAACGACGGCCAGT | 681 | CAGGAAACAGCTATGACC | 828 |
| Aminopeptidase P-like | XPNPEPL | AE114s24 | TGTAAAACGACGGCCAGT | 682 | CAGGAAACAGCTATGACC | 829 |
| Aminopeptidase P-like | XPNPEPL | AE114s23 | TGTAAAACGACGGCCAGT | 683 | CAGGAAACAGCTATGACC | 830 |
| Aminopeptidase P-like | XPNPEPL | AE114s22 | TGTAAAACGACGGCCAGT | 684 | CAGGAAACAGCTATGACC | 831 |
| Aminopeptidase P-like | XPNPEPL | AE114s18 | TGTAAAACGACGGCCAGT | 685 | CAGGAAACAGCTATGACC | 832 |
| Aminopeptidase P-like | XPNPEPL | AE114s14 | TGTAAAACGACGGCCAGT | 686 | CAGGAAACAGCTATGACC | 833 |
| Aminopeptidase P-like | XPNPEPL | AE114s13 | TGTAAAACGACGGCCAGT | 687 | CAGGAAACAGCTATGACC | 834 |
| Aminopeptidase P-like | XPNPEPL | AE114s16 | TGTAAAACGACGGCCAGT | 688 | CAGGAAACAGCTATGACC | 835 |
| Aminopeptidase P-like | XPNPEPL | AE114s12 | TGTAAAACGACGGCCAGT | 689 | CAGGAAACAGCTATGACC | 836 |
| Aminopeptidase P-like | XPNPEPL | AE114s45 | TGTAAAACGACGGCCAGT | 690 | CAGGAAACAGCTATGACC | 837 |
| Aminopeptidase P-like | XPNPEPL | AE114s44 | TGTAAAACGACGGCCAGT | 691 | CAGGAAACAGCTATGACC | 838 |
| Aminopeptidase P-like | XPNPEPL | AE114s43 | TGTAAAACGACGGCCAGT | 692 | CAGGAAACAGCTATGACC | 839 |
| Aminopeptidase P-like | XPNPEPL | AE114s42 | TGTAAAACGACGGCCAGT | 693 | CAGGAAACAGCTATGACC | 840 |
| Aminopeptidase P-like | XPNPEPL | AE114s4 | TGTAAAACGACGGCCAGT | 694 | CAGGAAACAGCTATGACC | 841 |
| Aminopeptidase P-like | XPNPEPL | AE114s35 | TGTAAAACGACGGCCAGT | 695 | CAGGAAACAGCTATGACC | 842 |
| Aminopeptidase P-like | XPNPEPL | AE114s34 | TGTAAAACGACGGCCAGT | 696 | CAGGAAACAGCTATGACC | 843 |
| Aminopeptidase P-like | XPNPEPL | AE114s36 | TGTAAAACGACGGCCAGT | 697 | CAGGAAACAGCTATGACC | 844 |
| Aminopeptidase P-like | XPNPEPL | AE114s38 | TGTAAAACGACGGCCAGT | 698 | CAGGAAACAGCTATGACC | 845 |
| Aminopeptidase P-like | XPNPEPL | AE114s33 | TGTAAAACGACGGCCAGT | 699 | CAGGAAACAGCTATGACC | 846 |
| Aminopeptidase P-like | XPNPEPL | AE114s32 | TGTAAAACGACGGCCAGT | 700 | CAGGAAACAGCTATGACC | 847 |
| Aminopeptidase P-like | XPNPEPL | AE114s5 | TGTAAAACGACGGCCAGT | 701 | CAGGAAACAGCTATGACC | 848 |
| Aminopeptidase P-like | XPNPEPL | AE114s3 | TGTAAAACGACGGCCAGT | 702 | CAGGAAACAGCTATGACC | 849 |
| Aminopeptidase P-like | XPNPEPL | AE114s7 | TGTAAAACGACGGCCAGT | 703 | CAGGAAACAGCTATGACC | 850 |
| Aminopeptidase P-like | XPNPEPL | AE114s6 | TGTAAAACGACGGCCAGT | 704 | CAGGAAACAGCTATGACC | 851 |
| Aminopeptidase P-like | XPNPEPL | AE114s9 | TGTAAAACGACGGCCAGT | 705 | CAGGAAACAGCTATGACC | 852 |
| Tissue kallikrein | KLK1 | AE115s13 | TGTAAAACGACGGCCAGT | 706 | CAGGAAACAGCTATGACC | 853 |
| Tissue kallikrein | KLK1 | AE115s12 | TGTAAAACGACGGCCAGT | 707 | CAGGAAACAGCTATGACC | 854 |
| Tissue kallikrein | KLK1 | AE115s11 | TGTAAAACGACGGCCAGT | 708 | CAGGAAACAGCTATGACC | 855 |
| Tissue kallikrein | KLK1 | AE115s14 | TGTAAAACGACGGCCAGT | 709 | CAGGAAACAGCTATGACC | 856 |
| Tissue kallikrein | KLK1 | AE115s1 | TGTAAAACGACGGCCAGT | 710 | CAGGAAACAGCTATGACC | 857 |
| Tissue kallikrein | KLK1 | AE115s4 | TGTAAAACGACGGCCAGT | 711 | CAGGAAACAGCTATGACC | 858 |
| Tissue kallikrein | KLK1 | AE115s3 | TGTAAAACGACGGCCAGT | 712 | CAGGAAACAGCTATGACC | 859 |
| Aminopeptidase P (membrane | XPNPEP2 | AE116s1 | TGTAAAACGACGGCCAGT | 713 | CAGGAAACAGCTATGACC | 860 |
| bound) | | | | | | |
| Aminopeptidase P (membrane | XPNPEP2 | AE116s2 | TGTAAAACGACGGCCAGT | 714 | CAGGAAACAGCTATGACC | 861 |
| bound) | | | | | | |
| Aminopeptidase P (membrane | XPNPEP2 | AE116s4 | TGTAAAACGACGGCCAGT | 715 | CAGGAAACAGCTATGACC | 862 |
| bound) | | | | | | |
| Aminopeptidase P (membrane | XPNPEP2 | AE116s3 | TGTAAAACGACGGCCAGT | 716 | CAGGAAACAGCTATGACC | 863 |
| bound) | | | | | | |
| Aminopeptidase P (membrane | XPNPEP2 | AE116s29 | TGTAAAACGACGGCCAGT | 717 | CAGGAAACAGCTATGACC | 864 |
| bound) | | | | | | |
| Aminopeptidase P (membrane | XPNPEP2 | AE116s30 | TGTAAAACGACGGCCAGT | 718 | CAGGAAACAGCTATGACC | 865 |
| bound) | | | | | | |
| Aminopeptidase P (membrane | XPNPEP2 | AE116s32 | TGTAAAACGACGGCCAGT | 719 | CAGGAAACAGCTATGACC | 866 |
| bound) | | | | | | |
| Aminopeptidase P (membrane | XPNPEP2 | AE116s33 | TGTAAAACGACGGCCAGT | 720 | CAGGAAACAGCTATGACC | 867 |
| bound) | | | | | | |
| Aminopeptidase P (membrane | XPNPEP2 | AE116s7 | TGTAAAACGACGGCCAGT | 721 | CAGGAAACAGCTATGACC | 868 |
| bound) | | | | | | |
| Aminopeptidase P (membrane | XPNPEP2 | AE116s18 | TGTAAAACGACGGCCAGT | 722 | CAGGAAACAGCTATGACC | 869 |
| bound) | | | | | | |
| Aminopeptidase P (membrane | XPNPEP2 | AE116s28 | TGTAAAACGACGGCCAGT | 723 | CAGGAAACAGCTATGACC | 870 |
| bound) | | | | | | |
| Aminopeptidase P (membrane | XPNPEP2 | AE116s21 | TGTAAAACGACGGCCAGT | 724 | CAGGAAACAGCTATGACC | 871 |
| bound) | | | | | | |
| Soluble guanylate cyclase 1, | GUCY1A2 | AE117s11 | TGTAAAACGACGGCCAGT | 725 | CAGGAAACAGCTATGACC | 872 |
| alpha-2 subunit | | | | | | |
| Soluble guanylate cyclase 1, | GUCY1A2 | AE117s13 | TGTAAAACGACGGCCAGT | 726 | CAGGAAACAGCTATGACC | 873 |
| alpha-2 subunit | | | | | | |
| Soluble guanylate cyclase 1, | GUCY1A2 | AE117s7 | TGTAAAACGACGGCCAGT | 727 | CAGGAAACAGCTATGACC | 874 |
| alpha-2 subunit | | | | | | |
| Soluble guanylate cyclase 1, | GUCY1A2 | AE117s6 | TGTAAAACGACGGCCAGT | 728 | CAGGAAACAGCTATGACC | 875 |
| alpha-2 subunit | | | | | | |
| Soluble guanylate cyclase 1, | GUCY1A2 | AE117s10 | TGTAAAACGACGGCCAGT | 729 | CAGGAAACAGCTATGACC | 876 |
| alpha-2 subunit | | | | | | |
| Soluble guanylate cyclase 1, | GUCY1A2 | AE117s9 | TGTAAAACGACGGCCAGT | 730 | CAGGAAACAGCTATGACC | 877 |
| alpha-2 subunit | | | | | | |
| Soluble guanylate cyclase 1, | GUCY1A2 | AE117s14 | TGTAAAACGACGGCCAGT | 731 | CAGGAAACAGCTATGACC | 878 |
| alpba-2 subunit | | | | | | |
| Soluble guanylate cyclase 1, | GUCY1A2 | AE117s15 | TGTAAAACGACGGCCAGT | 732 | CAGGAAACAGCTATGACC | 879 |
| alpha-2 subunit | | | | | | |
| Soluble guanylate cyclase 1, | GUCY1A2 | AE117s1 | TGTAAAACGACGGCCAGT | 733 | CAGGAAACAGCTATGACC | 880 |
| alpha-2 subunit | | | | | | |
| Soluble guanylate cyclase 1, | GUCY1A2 | AE117s2 | TGTAAAACGACGGCCAGT | 734 | CAGGAAACAGCTATGACC | 881 |
| alpha-2 subunit | | | | | | |
| Soluble guanylate cyclase 1, | GUCY1A2 | AE117s3 | TGTAAAACGACGGCCAGT | 735 | CAGGAAACAGCTATGACC | 882 |
| alpha-2 subunit | | | | | | |

TABLE VI

| | | | | | |
|---|---|---|---|---|---|
| | | | ORCHID_FOR_ | | |
| | | | WARD | | ORCHID_REVERSE |
| HGNC_ID | SNP_ID | ORCHID_FORWARD | (SEQ ID NO:) | ORCHID_REVERSE | (SEQ ID NO:) |

| | | | | | |
|---|---|---|---|---|---|
| C1S | AE111s1 | N/A | | N/A | |
| C1S | AE111s2 | N/A | | N/A | |
| C1S | AE111s13 | N/A | | N/A | |
| C1S | AE111s15 | N/A | | N/A | |
| C1S | AE111s17 | GTGAGCCTTACCTGTGGCAA | 1191 | AAACTCCAGGTGATCTTTAAGTCAGA | 1202 |
| C1S | AE111s19 | N/A | | N/A | |
| C1S | AE111s18 | N/A | | N/A | |
| C1S | AE111s20 | N/A | | N/A | |
| C1S | AE111s21 | ATCCAGACAGGTTATCTGCACC | 1192 | AATGCCCTGCCCTAAGGA | 1203 |
| C1S | AE111s22 | TACTTTACCTCCACAACTTCAAACC | 1193 | TAAGGAAGACACTCCCAATTCTGTT | 1204 |
| C1S | AE111s5 | N/A | | N/A | |
| C1S | AE111s6 | N/A | | N/A | |
| C1S | AE111s8 | N/A | | N/A | |
| ANPEP | AE112s57 | N/A | | N/A | |
| ANPEP | AE112s55 | N/A | | N/A | |
| ANPEP | AE112s56 | N/A | | N/A | |
| ANPEP | AE112s58 | N/A | | N/A | |
| ANPEP | AE112s44 | N/A | | N/A | |
| ANPEP | AE112s46 | N/A | | N/A | |
| ANPEP | AE112s42 | N/A | | N/A | |
| ANPEP | AE112s43 | N/A | | N/A | |
| ANPEP | AE112s41 | N/A | | N/A | |
| ANPEP | AE112s40 | N/A | | N/A | |
| ANPEP | AE112s36 | N/A | | N/A | |
| ANPEP | AE112s38 | N/A | | N/A | |
| ANPEP | AE112s35 | N/A | | N/A | |
| ANPEP | AE112s29 | N/A | | N/A | |
| ANPEP | AE112s33 | N/A | | N/A | |
| ANPEP | AE112s32 | N/A | | N/A | |
| ANPEP | AE112s30 | N/A | | N/A | |
| ANPEP | AE112s26 | N/A | | N/A | |
| ANPEP | AE112s22 | N/A | | N/A | |
| ANPEP | AE112s23 | N/A | | N/A | |
| ANPEP | AE112s21 | N/A | | N/A | |
| ANPEP | AE112s17 | N/A | | N/A | |
| ANPEP | AE112s18 | TCACATCCATCAGAGATGGC | 1194 | GGGGCTGCTGCCCATAAG | 1205 |
| ANPEP | AE112s19 | N/A | | N/A | |
| ANPEP | AE112s71 | N/A | | N/A | |
| ANPEP | AE112s72 | N/A | | N/A | |
| ANPEP | AE112s69 | N/A | | N/A | |
| ANPEP | AE112s68 | N/A | | N/A | |
| ANPEP | AE112s64 | N/A | | N/A | |
| ANPEP | AE112s63 | N/A | | N/A | |
| ANPEP | AE112s62 | N/A | | N/A | |
| ANPEP | AE112s61 | TTCTTCCTCACACCTTGCAGA | 1195 | ATAATGACCAGCAAAGAAGTTAAGGAT | 1206 |
| ANPEP | AE112s65 | N/A | | N/A | |
| ANPEP | AE112s53 | N/A | | N/A | |
| ANPEP | AE112s52 | N/A | | N/A | |
| ANPEP | AE112s13 | N/A | | N/A | |
| ANPEP | AE112s15 | N/A | | N/A | |
| ANPEP | AE112s1 | AATGTGAAGTTTCAGGGGTCTC | 1196 | AAGAAGTGGGAGGCTCATTGA | 1207 |
| ANPEP | AE112s5 | N/A | | N/A | |
| ANPEP | AE112s4 | N/A | | N/A | |
| ANPEP | AE112s3 | N/A | | N/A | |
| ANPEP | AE112s2 | N/A | | N/A | |
| ANPEP | AE112s9 | N/A | | N/A | |
| ANPEP | AE112s6 | N/A | | N/A | |
| ANPEP | AE112s7 | N/A | | N/A | |
| ANPEP | AE112s11 | N/A | | N/A | |
| ANPEP | AE112s12 | N/A | | N/A | |
| MEP1B | AE113s4 | N/A | | N/A | |
| MEP1B | AE113s3 | N/A | | N/A | |
| MEP1B | AE113s2 | N/A | | N/A | |
| MEP1B | AE113s1 | N/A | | N/A | |
| MEP1B | AE113s5 | N/A | | N/A | |
| MEP1B | AE113s34 | N/A | | N/A | |
| MEP1B | AE113s37 | N/A | | N/A | |
| MEP1B | AE113s38 | TTATCGCCTTAAAACATGTATTGACTTTA | 1197 | TTCTCCTTAGATGCCTTAGAAGACTAAGT | 1208 |
| MEP1B | AE113s39 | N/A | | N/A | |
| MEP1B | AE113s41 | N/A | | N/A | |
| MEP1B | AE113s40 | N/A | | N/A | |
| MEP1B | AE113s43 | N/A | | N/A | |
| MEP1B | AE113s42 | N/A | | N/A | |
| MEP1B | AE113s7 | N/A | | N/A | |
| MEP1B | AE113s6 | N/A | | N/A | |
| MEP1B | AE113s9 | N/A | | N/A | |
| MEP1B | AE113s8 | N/A | | N/A | |
| MEP1B | AE113s18 | N/A | | N/A | |
| MEP1B | AE113s14 | N/A | | N/A | |
| MEP1B | AE113s16 | N/A | | N/A | |
| MEP1B | AE113s21 | N/A | | N/A | |
| MEP1B | AE113s22 | N/A | | N/A | |
| MEP1B | AE113s24 | N/A | | N/A | |
| MEP1B | AE113s25 | N/A | | N/A | |
| MEP1B | AE113s26 | N/A | | N/A | |
| MEP1B | AE113s31 | N/A | | N/A | |
| MEP1B | AE113s30 | N/A | | N/A | |
| XPNPEPL | AE114s30 | N/A | | N/A | |
| XPNPEPL | AE114s31 | N/A | | N/A | |
| XPNPEPL | AE114s29 | N/A | | N/A | |
| XPNPEPL | AE114s28 | N/A | | N/A | |
| XPNPEPL | AE114s26 | N/A | | N/A | |
| XPNPEPL | AE114s25 | N/A | | N/A | |
| XPNPEPL | AE114s24 | N/A | | N/A | |
| XPNPEPL | AE114s23 | N/A | | N/A | |
| XPNPEPL | AE114s22 | N/A | | N/A | |
| XPNPEPL | AE114s18 | N/A | | N/A | |
| XPNPEPL | AE114s14 | N/A | | N/A | |
| XPNPEPL | AE114s13 | N/A | | N/A | |
| XPNPEPL | AE114s16 | N/A | | N/A | |
| XPNPEPL | AE114s12 | N/A | | N/A | |
| XPNPEPL | AE114s45 | N/A | | N/A | |
| XPNPEPL | AE114s44 | N/A | | N/A | |
| XPNPEPL | AE114s43 | N/A | | N/A | |
| XPNPEPL | AE114s42 | N/A | | N/A | |
| XPNPEPL | AE114s41 | N/A | | N/A | |
| XPNPEPL | AE114s35 | N/A | | N/A | |
| XPNPEPL | AE114s34 | N/A | | N/A | |
| XPNPEPL | AE114s36 | N/A | | N/A | |
| XPNPEPL | AE114s38 | N/A | | N/A | |
| XPNPEPL | AE114s33 | N/A | | N/A | |
| XPNPEPL | AE114s32 | N/A | | N/A | |
| XPNPEPL | AE114s5 | N/A | | N/A | |
| XPNPEPL | AE114s3 | TTCCTGGATCTCCTCTCCCC | 1198 | CAGACTGGGCAAAAATTAACCA | 1209 |
| XPNPEPL | AE114s7 | N/A | | N/A | |
| XPNPEPL | AE114s6 | N/A | | N/A | |
| XPNPEPL | AE114s9 | N/A | | N/A | |
| KLK1 | AE115s13 | N/A | | N/A | |
| KLK1 | AE115s12 | N/A | | N/A | |
| KLK1 | AE115s11 | N/A | | N/A | |
| KLK1 | AE115s14 | N/A | | N/A | |
| KLK1 | AE115s1 | GGACTTTATTCAAGAATAGGAGATAAGTA | 1199 | CTGTTGTACGTATTATAGTTTATTTAACTG | 1210 |
| | | CAC | | ACCA | |
| KLK1 | AE115s4 | N/A | | N/A | |
| KLK1 | AE115s3 | N/A | | N/A | |
| XPNPEP2 | AE116s1 | AAGGCAGCTCCTAGTAAATGCTCTA | 1200 | ATTGGGTCTGTTTGTCTCTCTAAAGC | 1211 |
| XPNPEP2 | AE116s2 | TTTTTCCCAGCCCCCTCTC | 1201 | AGAGCCCAGAGTGTGGGAG | 1212 |
| XPNPEP2 | AE116s4 | N/A | | N/A | |
| XPNPEP2 | AE116s3 | N/A | | N/A | |
| XPNPEP2 | AE116s29 | N/A | | N/A | |
| XPNPEP2 | AE116s30 | N/A | | N/A | |
| XPNPEP2 | AE116s32 | N/A | | N/A | |
| XPNPEP2 | AE116s33 | N/A | | N/A | |
| XPNPEP2 | AE116s7 | N/A | | N/A | |
| XPNPEP2 | AE116s18 | N/A | | N/A | |
| XPNPEP2 | AE116s28 | N/A | | N/A | |
| XPNPEP2 | AE116s21 | N/A | | N/A | |
| GUCY1A2 | AE117s11 | N/A | | N/A | |
| GUCY1A2 | AE117s13 | N/A | | N/A | |
| GUCY1A2 | AE117s7 | N/A | | N/A | |
| GUCY1A2 | AE117s6 | N/A | | N/A | |
| GUCY1A2 | AE117s10 | N/A | | N/A | |
| GUCY1A2 | AE117s9 | N/A | | N/A | |
| GUCY1A2 | AE117s14 | N/A | | N/A | |
| GUCY1A2 | AE117s15 | N/A | | N/A | |
| GUCY1A2 | AE117s1 | N/A | | N/A | |
| GUCY1A2 | AE117s2 | N/A | | N/A | |
| GUCY1A2 | AE117s3 | N/A | | N/A | |

TABLE VII

| HGNC_ID | SNP_ID | GBS_LEFT | GBS_LEFT (SEQ ID NO:) | GBS_RIGHT | GBS_RIGHT (SEQ ID NO:) |
|---|---|---|---|---|---|
| C1S | AE111s1 | TGTAAAACGACGGCCAGTTTTATGGACCAGGATA | 897 | CAGGAAACAGCTATGACCTCAGTCCCTTGTCTGCA | 1044 |
| | | ACCCC | | ATTTC | |
| C1S | AE111s2 | TGTAAAACGACGGCCAGTTCTGCTCCTCGTTTGTA | 898 | CAGGAAACAGCTATGACCAGCATTAAGCAATCGG | 1045 |
| | | AGGA | | TGAAA | |
| C1S | AE111s13 | TGTAAAACGACGGCCAGTTCTACCACCTCAGCCTT | 899 | CAGGAAACAGCTATGACCAGAAAGCCAGGACCAA | 1046 |
| | | TTGA | | GAGAC | |
| C1S | AE111s15 | TGTAAAACGACGGCCAGTGTGGGGATTGTTACTG | 900 | CAGGAAACAGCTATGACCCAGGCATATCCCAGTG | 1047 |
| | | CTCCT | | AGGTA | |
| C1S | AE111s17 | TGTAAAACGACGGCCAGTCCTCATTCAATGTGTTG | 901 | CAGGAAACAGCTATGACCACTGAAGAAGGGAGGC | 1048 |
| | | CTCA | | TCTGT | |
| C1S | AE111s19 | TGTAAAACGACGGCCAGTATCATGGAGGAAATAT | 902 | CAGGAAACAGCTATGACCAGGGCTCAACTCTGGA | 1049 |
| | | TCCGG | | ATCAT | |
| C1S | AE111s18 | TGTAAAACGACGGCCAGTAAGAAGTGCCAAATGA | 903 | CAGGAAACAGCTATGACCCGGATCTTTAAGCAAT | 1050 |
| | | AGGCT | | AGGCC | |
| C1S | AE111s20 | TGTAAAACGACGGCCAGTTATCACCAACCAAAGA | 904 | CAGGAAACAGCTATGACCATAGGTGTGAGCCACT | 1051 |
| | | GGCAC | | GCACT | |
| C1S | AE111s21 | TGTAAAACGACGGCCAGTGACAATTTCAGTCCGCT | 905 | CAGGAAACAGCTATGACCGACCTGAGACTGCAGA | 1052 |
| | | CATC | | CAGCT | |
| C1S | AE111s22 | TGTAAAACGACGGCCAGTCTCCATGACTCACAAG | 906 | CAGGAAACAGCTATGACCTGAGGACAACTGGACG | 1053 |
| | | GGAGA | | ATTTT | |
| C1S | AE111s5 | TGTAAAACGACGGCCAGTAGTCCACAGCTACCAG | 907 | CAGGAAACAGCTATGACCCTTTCCAAGAAAGGGG | 1054 |
| | | ACGAA | | CTATG | |
| C1S | AE111s6 | TGTAAAACGACGGCCAGTAGGCCCTGTTTTTAGCA | 908 | CAGGAAACAGCTATGACCAGTGTGGCCTGTGTTCT | 1055 |
| | | GTTC | | CTGT | |
| C1S | AE111s8 | TGTAAAACGACGGCCAGTTGTGGGTTTCTCCACTT | 909 | CAGGAAACAGCTATGACCCCAACAATGTATGTTG | 1056 |
| | | TCAC | | GGTCC | |
| ANPEP | AE112s57 | TGTAAAACGACGGCCAGTCCCTCTTTCACCTTTCC | 910 | CAGGAAACAGCTATGACCCTCCAGCTGCCATCAG | 1057 |
| | | CTAA | | AATAG | |
| ANPEP | AE112s55 | TGTAAAACGACGGCCAGTTGCCTGTGAGCCAGTCT | 911 | CAGGAAACAGCTATGACCCAGCTGCTGTATTGAC | 1058 |
| | | AGTT | | CCACT | |
| ANPEP | AE112s56 | TGTAAAACGACGGCCAGTCATCAAGTGGGTGAAG | 912 | CAGGAAACAGCTATGACCTTCATTGTCCATCGAGA | 1059 |
| | | GAGAA | | GCTT | |
| ANPEP | AE112s58 | TGTAAAACGACGGCCAGTCATCAAGTGGGTGAAG | 913 | CAGGAAACAGCTATGACCTfCATTGTCCATCGAGA | 1060 |
| | | GAGAA | | GCTT | |
| ANPEP | AE112s44 | TGTAAAACGACGGCCAGTGCAGGTAAGAAGTCAT | 914 | CAGGAAACAGCTATGACCGGTAACGGGTACATGG | 1061 |
| | | TCCCC | | AGGTT | |
| ANPEP | AE112s46 | TGTAAAACGACGGCCAGTCTCGTTCTCCTTCTCCA | 915 | CAGGAAACAGCTATGACCGGTAACGGGTACATGG | 1062 |
| | | ACCT | | AGGTT | |
| ANPEP | AE112s42 | TGTAAAACGACGGCCAGTTCTCTCCCTCCCTGCAG | 916 | CAGGAAACAGCTATGACCGGTAACGGGTACATGG | 1063 |
| | | TTAT | | AGGTT | |
| ANPEP | AE112s43 | TGTAAAAGGACGGCCAGTTCTCTCCCTCCCTGCAG | 917 | CAGGAAACAGCTATGACCGGTAACGGGTACATGG | 1064 |
| | | TTAT | | AGGTT | |
| ANPEP | AE112s41 | TGTAAAACGACGGCCAGTCAGGAATGGAAAGACA | 918 | CAGGAAACAGCTATGACCTTCTTACCTGCTGCAGC | 1065 |
| | | AGCTG | | TCAT | |
| ANPEP | AE112s40 | TGTAAAACGACGGCCAGTGACTTAATCCGGAAGC | 919 | CAGGAAACAGCTATGACCCACAGCTTGAACACAG | 1066 |
| | | AGGAC | | GTCTG | |
| ANPEP | AE112s36 | TGTAAAACGACGGCCAGTGGGACAGTCCAAGTTC | 920 | CAGGAAACAGCTATGACCTGGCTGATTTTTGTCCA | 1067 |
| | | TCTCC | | CTTC | |
| ANPEP | AE112s38 | TGTAAAACGACGGCCAGTTGTGACCCTGCTTAGGT | 921 | CAGGAAACAGCTATGACCCTGACACCCATTCCAC | 1068 |
| | | GACT | | AGACT | |
| ANPEP | AE112s35 | TGTAAACGACGGCCAGTCGCTAGGACTCCTTCCT | 922 | CAGGAAACAGCTATGACCACAAAGTCCCAGACCA | 1069 |
| | | TCAT | | GACCT | |
| ANPEP | AE112s29 | TGTAAAACGACGGCCAGTCACCGTCTACTGCAAC | 923 | CAGGAAACAGCTATGACCTCAGGTAAAGGCTAAG | 1070 |
| | | GCTAT | | AGGCC | |
| ANPEP | AE112s33 | TGTAAAACGACGGCCAGTGTCCTCAGCAGAACCC | 924 | CAGGAAACAGCTATGACCAATACTGCCTCCACCTC | 1071 |
| | | TGTC | | AACA | |
| ANPEP | AE112s32 | TGTAAAACGACGGCCAGTACCCCAATAATAACCC | 925 | CAGGAAACAGCTATGACCGACTCACCTGTTCAGG | 1072 |
| | | GTGAG | | ATCCA | |
| ANPEP | AE112s30 | TGTAAAACGACGGCCAGTACCCCAATAATAACCC | 926 | CAGGAAACAGCTATGACCGACTCACCTGITCAGG | 1073 |
| | | GTGAG | | ATCCA | |
| ANPEP | AE112s26 | TGTAAAACGACGGCCAGTGAAATGGTGACCCGTA | 927 | CAGGAAACAGCTATGACCACTTCCAAACCCATGA | 1074 |
| | | GACAA | | GAGCT | |
| ANPEP | AE112s22 | TGTAAAACGACGGCCAGTTCATTAATGACGCCTTC | 928 | CAGGAAACAGCTATGACCAAAGCTTGGTGCTGTG | 1075 |
| | | AACC | | GAGTA | |
| ANPEP | AE112s23 | TGTAAAACGACGGCCAGTTCATTAATGACGCCTTC | 929 | CAGGAAACAGCTATGACCAAAGCTTGGTGCTGTG | 1076 |
| | | AACC | | GAGTA | |
| ANPEP | AE112s21 | TGTAAAACGACGGCCAGTCTCAGCTGCAGAGAGA | 930 | CAGGAAACAGCTATGACCCATCTGTGAAATGGGT | 1077 |
| | | CCACT | | GTGTG | |
| ANPEP | AE112s17 | TGTAAAACGACGGCCAGTCCCATCACATCCATCA | 931 | CAGGAAACAGCTATGACCTGGCCTAGGATTCTCTC | 1078 |
| | | GAGAT | | CTTT | |
| ANPEP | AE112s18 | TGTAAAACGACGGCCAGTGTAGACAGACCCTCCC | 932 | CAGGAAACAGCTATGACCCAGCAAAGTGGAGATT | 1079 |
| | | TCCAG | | GGAAC | |
| ANPEP | AE112s19 | TGTAAAACGACGGCCAGTGTAGACAGACCCTCCC | 933 | CAGGAAACAGCTATGACCCAGCAAAGTGGAGATT | 1080 |
| | | TCCAG | | GGAAC | |
| ANPEP | AE112s71 | TGTAAAACGACGGCCAGTTCATGGTGCTGAATGA | 934 | CAGGAAACAGCTATGACCCTTGAATACGTCCTCG | 1081 |
| | | TGTGT | | GACAG | |
| ANPEP | AE112s72 | TGTAAAACGACGGCCAGTTCATGGTGCTGAATGA | 935 | CAGGAAACAGCTATGACCCTTGAATACGTCCTCG | 1082 |
| | | TGTGT | | GACAG | |
| ANPEP | AE112s69 | TGTAAAACGACGGCCAGTGGGAACCTGGTGACCA | 936 | CAGGAAACAGCTATGACCCTGTAGGAGATGGCGT | 1083 |
| | | TAGAG | | CAAAC | |
| ANPEP | AE112s68 | TGTAAAACGACGGCCAGTACTCCCAAAATCAGGT | 937 | CAGGAAACAGCTATGACCGATTTCAACACCATCG | 1084 |
| | | GAGTG | | TGACC | |
| ANPEP | AE112s64 | TGTAAAACGACGGCCAGTCTGCTTCTTCCTCACAC | 938 | CAGGAAACAGCTATGACCTCATGAGCAATCACAG | 1085 |
| | | CTTG | | TGACC | |
| ANPEP | AE112s63 | TGTAAAACGACGGCCAGTAGGTTTAGCTTGATTGG | 939 | CAGGAAACAGCTATGACCGTAGGTCACCAGTCCC | 1086 |
| | | CCTC | | CAGTT | |
| ANPEP | AE112s62 | TGTAAAACGACGGCCAGTAGGTTTAGCTTGATTGG | 940 | CAGGAAACAGCTATGACCGTAGGTCACCAGTCCC | 1087 |
| | | CCTC | | CAGTT | |
| ANPEP | AE112s61 | TGTAAAACGACGGCCAGTAGGTTTAGCTTGATTGG | 941 | CAGGAAACAGCTATGACCGTAGGTCACCAGTCCC | 1088 |
| | | CCTC | | CAGTT | |
| ANPEP | AE112s65 | TGTAAAACGACGGCCAGTCAGACGGTGGCTATGA | 942 | CAGGAAACAGCTATGACCGCAAAGAAGTTAAGGA | 1089 |
| | | TGATT | | TGGGG | |
| ANPEP | AE112s53 | TGTAAAACGACGGCCAGTACCCTCTAACCTTCCTG | 943 | CAGGAAACAGCTATGACCACATTCCAGTTGGGGT | 1090 |
| | | TTGG | | CTTCT | |
| ANPEP | AE112s52 | TGTAAAACGACGGCCAGTGAGAGAAACTCACCCG | 944 | CAGGAAACAGCTATGACCACTCACCTTTGGGAAG | 1091 |
| | | AGGTC | | CATGT | |
| ANPEP | AE112s13 | TGTAAAACGACGGCCAGTGGACAGCCAGTATGAG | 945 | CAGGAAACAGCTATGACCGACCTCGGGTGAGTTT | 1092 |
| | | ATGGA | | CTCTC | |
| ANPEP | AE112s15 | TGTAAAACGACGGCCAGTCACCACCTTGGACCAA | 946 | CAGGAAACAGCTATGACCTCCATCTCATACTGGCT | 1093 |
| | | AGTAA | | GTCC | |
| ANPEP | AE112s1 | TGTAAAACGACGGCCAGTAGCTCTCAAGCAGATC | 947 | CAGGAAACAGCTATGACCGCCCAGGGACTTGGAA | 1094 |
| | | AATGC | | ATATA | |
| ANPEP | AE112s5 | TGTAAAACGACGGCCAGTTACCTGCTCAAGGTCA | 948 | CAGGAAACAGCTATGACCCCCGGCTCCTATTAAG | 1095 |
| | | CAGCT | | AACAC | |
| ANPEP | AE112s4 | TGTAAAACGACGGCCAGTCAGCCGGGAATGATTT | 949 | CAGGAAACAGCTATGACCCCCGGCTCCTATTAAG | 1096 |
| | | TTAA | | AACAC | |
| ANPEP | AE112s3 | TGTAAAACGACGGCCAGTCAGGCTGGTCCTTGAAC | 950 | CAGGAAACAGCTATGACCCTTCAAGAGCAGGGAG | 1097 |
| | | TCCTA | | TTCCT | |
| ANPEP | AE112s2 | TGTAAAACGACGGCCAGTATCTCTGCCTCCCGGAT | 951 | CAGGAAACAGCTATGACCCTTTCCAGCTGTGACCT | 1098 |
| | | T | | TGAG | |
| ANPEP | AE112s9 | TGTAAAACGACGGCCAGTGAAGGGAACGATTTTC | 952 | CAGGAAACAGCTATGACCTGCCTCTCAGGTTTGTT | 1099 |
| | | TTCTTTT | | GAAC | |
| ANPEP | AE112s6 | TGTAAAACGACGGCCAGTGAAGGGAACGATTTTC | 953 | CAGGAAACAGCTATGACCTGCCTCTCAGGTTTGTT | 1100 |
| | | TTCTTTT | | GAAC | |
| ANPEP | AE112s7 | TGTAAAACGACGGCCAGTGCAATTGTTGAGCTTTG | 954 | CAGGAAACAGCTATGACCTGTTGAACAGATTTAG | 1101 |
| | | GGTA | | TGAGAAAACA | |
| ANPEP | AE112s11 | TGTAAAACGACGGCCAGTGATGGGGAAAGTGACA | 955 | CAGGAAACAGCTATGACCTAGGAGTTCAAGACCA | 1102 |
| | | ATGAA | | GCCTG | |
| ANPEP | AE112s12 | TGTAAAACGACGGCCAGTGATGGGGAAAGTGACA | 956 | CAGGAAACAGCTATGACCTAGGAGTTCAAGACCA | 1103 |
| | | ATGAA | | GCCTG | |
| MEP1B | AE113s4 | TGTAAAACGACGGCCAGTTTAGCACAAAATGGGG | 957 | CAGGAAACAGCTATGACCCATGGTGGCTCACATG | 1104 |
| | | TGAAG | | TGTAA | |
| MEP1B | AE113s3 | TGTAAAACGACGGCCAGTATGGAGTGTCATTCTGT | 958 | CAGGAAACAGCTATGACCCTCAGTTCCCATGGCCTT | 1105 |
| | | TGCC | | CATA | |
| MEP1B | AE113s2 | TGTAAAACGACGGCCAGTATGGAGTGTCATTCTGT | 959 | CAGGAAACAGCTATGACCCTCAGTTCCCATGGCTT | 1106 |
| | | TGCC | | CATA | |
| MEP1B | AE113s1 | TGTAAAACGACGGCCAGTATGGAGTGTCATTCTGT | 960 | CAGGAAACAGCTATGACCCTCAGTTCCATGGCTT | 1107 |
| | | TGCC | | CATA | |
| MEP1B | AE113s5 | TGTAAAACGACGGCCAGTCTTTTATGACCCATGTC | 961 | CAGGAAACAGCTATGACCTGCCTGGCTGAGACTT | 1108 |
| | | CCAC | | CTTAA | |
| MEP1B | AE113s34 | TGTAAAACGACGGCCAGTATCAAAGATGTAGATG | 962 | CAGGAAACAGCTATGACCATGTGTCACCATGATTC | 1109 |
| | | GCGGA | | TGCA | |
| MEP1B | AE113s37 | TGTAAAACGACGGCCAGTTGCATTTATTTTTGACA | 963 | CAGGAAACAGCTATGACCAACGTTGGTTGAACTC | 1110 |
| | | GGCA | | ATTGC | |
| MEP1B | AE113s38 | TGTAAAACGACGGCCAGTTATATTGCAGTGGCCTG | 964 | CAGGAAACAGCTATGACCGCAACTAGCAAGAAAG | 1111 |
| | | CTTC | | TCCCC | |
| MEP1B | AE113s39 | TGTAAAACGACGGCCAGTGTCCAAACTATGTCAC | 965 | CAGGAAACAGCTATGACCGACATAGTCATCCCGG | 1112 |
| | | CTGGG | | TCAGA | |
| MEP1B | AE113s41 | TGTAAAACGACGGCCAGTAGTTCAACACGAGTTC | 966 | CAGGAAACAGCTATGACCGCAAAACTAATTTGGC | 1113 |
| | | CTCCA | | CACAG | |
| MEP1B | AE113s40 | TGTAAAACGACGGCCAGTAGTTCAACACGAGTTC | 967 | CAGGAAACAGCTATGACCGCAAAACTAATTTGGC | 1114 |
| | | CTCCA | | CACAG | |
| MEP1B | AE113s43 | TGTAAAACGACGGCCAGTGGGTTCTGCAAATAAA | 968 | CAGGAAACAGCTATGACCTCACTCCTGTTACCTTG | 1115 |
| | | GAGGG | | GCAC | |
| MEP1B | AE113s42 | TGTAAAACGACGGCCAGTAGGAGATAATGCTGAC | 969 | CAGGAAACAGCTATGACCAGATAAGAGGCCTTGC | 1116 |
| | | TGGCA | | AGGAG | |
| MEP1B | AE113s7 | TGTAAAACGACGGCCAGTTAGCTTTTGCAAGTGCC | 970 | CAGGAAACAGCTATGACCAAGGGTTAAATTGCCA | 1117 |
| | | AGAT | | TCCAC | |
| MEP1B | AE113s6 | TGTAAAACGACGGCCAGTTAGCTTTTGCAAGTGCC | 971 | CAGGAAACAGCTATGACCAAGGGTTAAATTGCCA | 1118 |
| | | AGAT | | TCCAC | |
| MEP1B | AE113s9 | TGTAAAACGACGGCCAGTGGTTTTGGATGACCCTT | 972 | CAGGAAACAGCTATGACCATCCCTGCATTAGTCAC | 1119 |
| | | TGTC | | ATGG | |
| MEP1B | AE113s8 | TGTAAAACGACGGCCAGTTTTTGCAGAAATACCC | 973 | CAGGAAACAGCTATGACCCCAAAAGTGTCATTGT | 1120 |
| | | ACTGG | | GGCTT | |
| MEP1B | AE113s18 | TGTAAAACGACGGCCAGTTTCACATTGCCTGTTCT | 974 | CAGGAAACAGCTATGACCAATCAGTGGCCACAAC | 1121 |
| | | TTCC | | AAAAA | |
| MEP1B | AE113s14 | TGTAAAACGACGGCCAGTTTCACATTGCCTGTTCT | 975 | CAGGAAACAGCTATGACCAATCAGTGGCCACAAC | 1122 |
| | | TTCC | | AAAAA | |
| MEP1B | AE113s16 | TGTAAAACGACGGCCAGTCAGTGCCTTTATAACCC | 976 | CAGGAAACAGCTATGACCTTCCGCTATAGCTTCAT | 1123 |
| | | ACGA | | GTGG | |
| MEP1B | AE113s21 | TGTAAAACGACGGCCAGTCAGTGCCTTTATAACCC | 977 | CAGGAAACAGCTATGACCTTCCGCTATAGCTTCAT | 1124 |
| | | AGGA | | GTGG | |
| MEP1B | AE113s22 | TGTAAAACGACGGCCAGTACTTAAGATTGGGCTT | 978 | CAGGAAACAGCTATGACCCCTTACCTGCACTCAGC | 1125 |
| | | GGCAT | | TTTG | |
| MEP1B | AE113s24 | TGTAAAACGACGGCCAGTTCTCCAGTCCAGAGTG | 979 | CAGGAAACAGCTATGACCTCAAATTTGGTCGATTT | 1126 |
| | | GGTAA | | GAGC | |
| MEP1B | AE113s25 | TGTAAAACGACGGCCAGTGAGTCATGCTCTCAAC | 980 | CAGGAAACAGCTATGACCGTTGTCCCAGCATAGG | 1127 |
| | | ATGCA | | AAACA | |
| MEP1B | AE113s26 | TGTAAAACGACGGCCAGTATGCTGATCATCACCCT | 981 | CAGGAAACAGCTATGACCGCAAAATCAGTTGTGC | 1128 |
| | | TGTC | | CTTTC | |
| MEP1B | AE113s31 | TGTAAAACGACGGCCAGTGCGAATGTATGTGTTC | 982 | CAGGAAACAGCTATGACCTAGGCGAAATCCATGA | 1129 |
| | | ACCCT | | TGAAG | |
| MEP1B | AE113s30 | TGTAAAACGACGGCCAGTTCTTTCCCCTTTTAGCA | 983 | CAGGAAACAGCTATGACCTCCCATTGGGTGTTTCT | 1130 |
| | | GCAT | | AGTG | |
| XPNPEPL | AE114s30 | TGTAAAACGACGGCCAGTCTTCCTTCCTCCAGAGC | 984 | CAGGAAACAGCTATGACCCAGAAGCCATGAAGTC | 1131 |
| | | ATTT | | TGGTC | |
| XPNPEPL | AE114s31 | TGTAAAACGACGGCCAGTTCAGGTGGTAATTGTTG | 985 | CAGGAAACAGCTATGACCCAGGAGWfCTCACAGA | 1132 |
| | | AGCC | | GTCCG | |
| XPNPEPL | AE114s29 | TGTAAAACGACGGCCAGTCATGTTTCCCCTGAGTG | 986 | CAGGAAACAGCTATGACCGCGGCATCAGTTACAA | 1133 |
| | | GTTA | | AACAT | |
| XPNPEPL | AE114s28 | TGTAAAACGACGGCCAGTATGTTTTGTAACTGATG | 987 | CAGGAAACAGCTATGACCCTTGTGCTGTGCTGAA | 1134 |
| | | CCGC | | ATCTG | |
| XPNPEPL | AE114s26 | TGTAAAACGACGGCCAGTAAACAAGAAAAAGAGC | 988 | CAGGAAACAGCTATGACCGGGTCATCTCTCTGAC | 1135 |
| | | CTGCC | | AGTGC | |
| XPNPEPL | AE114s25 | TGTAAAACGACGGCCAGTGCCCTAACCTTAGCAG | 989 | CAGGAAACAGCTATGACCTCTCACTTGTCCACATG | 1136 |
| | | AATGC | | CTTG | |
| XPNPEPL | AE114s24 | TGTAAAACGACGGCCAGTGCCCTAACCTTAGCAG | 990 | CAGGAAACAGCTATGACCTCTCACTTGTCCACATG | 1137 |
| | | AATGC | | CTTG | |
| XPNPEPL | AE114s23 | TGTAAAACGACGGCCAGTCCCTTACTTCATAAGGC | 991 | CAGGAAACAGCTATGACCGTAAACCAGGACTGCT | 1138 |
| | | CCTG | | GTGGA | |
| XPNPEPL | AE114s22 | TGTAAAACGACGGCCAGTAAACTTCCAGCCAAGG | 992 | CAGGAAACAGCTATGACCCTTGTCCCTGGATGAG | 1139 |
| | | ACTGT | | GTGTA | |
| XPNPEPL | AE114s18 | TGTAAAACGACGGCCAGTTAAGAAGAGGCTCCCA | 993 | CAGGAAACAGCTATGACCTIGCTAGTTGAATGAG | 1140 |
| | | AAAGG | | GCTGG | |
| XPNPEPL | AE114s14 | TGTAAAACGACGGCCAGTTGGGGATTGGGAGAAA | 994 | CAGGAAACAGCTATGACCGGAGTTTCGCAGGTAA | 1141 |
| | | ATAAA | | GGATC | |
| XPNPEPL | AE114s13 | TGTAAAACGACGGCCAGTAAATTGTTGGGAAGCT | 995 | CAGGAAACAGCTATGACCGTGAGAGCCTTTGGGA | 1142 |
| | | CAGGT | | GTTCT | |
| XPNPEPL | AE114s16 | TGTAAAACGACGGCCAGTGATCCTTACCTGCGAA | 996 | CAGGAAACAGCTATGACCTAGGAGGGGATGGTTT | 1143 |
| | | ACTCC | | TGAAT | |
| XPNPEPL | AE114s12 | TGTAAAACGACGGCCAGTACCACAGAGAAACCAC | 997 | CAGGAAACAGCTATGACCnGGCCATTAATTTCTT | 1144 |
| | | CCTGT | | GCTC | |
| XPNPEPL | AE114s45 | TGTAAAACGACGGCCAGTGGCCCAAAATCTTTCTT | 998 | CAGGAAACAGCTATGACCGTATGCCTTACACCCCC | 1145 |
| | | CATC | | ATCT | |
| XPNPEPL | AE114s44 | TGTAAAACGACGGCCAGTCAATAACTCCTCCTGG | 999 | CAGGAAACAGCTATGACCTAGCTATATGGGTCGC | 1146 |
| | | AAGGC | | CAGTG | |
| XPNPEPL | AE114s43 | TGTAAAACGACGGCCAGTCTTGTCACTGACCCACA | 1000 | CAGGAAACAGCTATGACCAGATTTCTGGCTTGGC | 1147 |
| | | CCTT | | AGATT | |
| XPNPEPL | AE114s42 | TGTAAAACGACGGCCAGTCTGGGTTTATTTGCATT | 1001 | CAGGAAACAGCTATGACCATCAGATGTGGAGCAC | 1148 |
| | | GTGG | | AATCC | |
| XPNPEPL | AE114s41 | TGTAAAACGACGGCCAGTCACAGATACCCAGAGA | 1002 | CAGGAAACAGCTATGACCTCAAGTGGCTTGGACA | 1149 |
| | | CCCAA | | CTTCT | |
| XPNPEPL | AE114s35 | TGTAAAACGACGGCCAGTCAAAGCCAAACTAAGT | 1003 | CAGGAAACAGCTATGACCGGAAGAAAATGGCCAA | 1150 |
| | | GGCAA | | AGTTC | |
| XPNPEPL | AE114s34 | TGTAAAACGACGGCCAGTCAAAGCCAAACTAAGT | 1004 | CAGGAAACAGCTATGACCGGAAGAAAATGGCCAA | 1151 |
| | | GGCAA | | AGTTC | |
| XPNPEPL | AE114s36 | TGTAAAACGACGGCCAGTCAACGAGGTTCTCCTTG | 1005 | CAGGAAACAGCTATGACCGTCCCTTTCTGCTTGGG | 1152 |
| | | ACAG | | TAAG | |
| XPNPEPL | AE114s38 | TGTAAAACGACGGCCAGTCAACGAGGTTCTCCTTG | 1006 | CAGGAAACAGCTATGACCGTCCCTTTCTGCTTGGG | 1153 |
| | | ACAG | | TAAG | |
| XPNPEPL | AE114s33 | TGTAAAACGACGGCCAGTTCCTTGGATTCTCAAAA | 1007 | CAGGAAACAGCTATGACCCCCAGAGGTTTCTTTGG | 1154 |
| | | GGGT | | AGTC | |
| XPNPEPL | AE114s32 | TGTAAAACGACGGCCAGTCCATCGAATCCAGAGA | 1008 | CAGGAAACAGCTATGACCAAAGGGAACAGCTCTC | 1155 |
| | | CAAAA | | TCTGC | |
| XPNPEPL | AE114s5 | TGTAAAACGACGGCCAGTATCGGTTCGGTCACAT | 1009 | CAGGAAACAGCTATGACCTAGCTGAACTTTTCTGG | 1156 |
| | | ACTCA | | CCAC | |
| XPNPEPL | AE114s3 | TGTAAAACGACGGCCAGTCCCACTCCCAGTTAGGT | 1010 | CAGGAAACAGCTATGACCTCACCCACAAATGTTG | 1157 |
| | | CPTC | | TCTACC | |
| XPNPEPL | AE114s7 | TCTAAAACCACCCCCACTCCCACTCCCACTTAGGT | 1011 | CACCAAACACCTATCACCTCACCCACAAATCTTC | 1158 |
| | | CTTC | | TCTAGG | |
| XPNPEPL | AE114s6 | TCTAAAACCACCCCCACTAACCACCACAACTCTA | 1012 | CACCAAACACCTATCACCCTCCTCACTCCAAGTC | 1159 |
| | | TCCTCA | | ATTCC | |
| XPNPEPL | AE114s9 | TCTAAAACCACCGCCACTACTCACCTCACACCAC | 1013 | CAGGAAACAGCTATGACCTTTGACTCCTAGTGGAC | 1160 |
| | | ACCAC | | CCAA | |
| KLK1 | AE115s13 | TGTAAAACCACGGCCACTAACACATTACCACCAC | 1014 | CACCAAACACCTATCACCCCACTCTCTCGACCTC | 1161 |
| | | CAACG | | AAAAT | |
| KLK1 | AE115s12 | TCTAAAACCACCCCCACTAACACATTACCACCAC | 1015 | CACCAAACACCTATCACCCCACTCTCTCCACCTC | 1162 |
| | | CAACC | AAAAT | | |
| KLK1 | AE115s11 | TCTAAAACCACCCCCACTCTCAACCACATCCCTC | 1016 | CACCAAACAGCTATCACCAATTCTATCTCGGGCC | 1163 |
| | | CTTAG | ACACT | | |
| KLK1 | AE115s14 | TCTAAAACCACCGCCACTCCTCACCACACACCTC | 1017 | CACCAAACACCTATCACCCCTCACACACCCTGCT | 1164 |
| | | TCTTT | CATAC | | |
| KLK1 | AE115s1 | TGTAAAACGACGGCCAGTTCCCCAAAGCTACTGTT | 1018 | CACGAAACACCTATCACCAAATGTCATGTTCAAG | 1165 |
| | | CCTT | GTCCC | | |
| KLK1 | AE115s4 | TCTAAAACCACGGCCACTGCCTACACATCACCAC | 1019 | CAGGAAACAGCTATCACCAAATGTGATGTTCAAG | 1166 |
| | | ATTCC | | CTCCC | |
| KLK1 | AE115s3 | TGTAAAACGACGGCCAGTCAGGCTAACCACAGAT | 1020 | CAGGAAACAGCTATGACCTTTCTTAGCCAGGTCCC | 1167 |
| | | TCCAA | | TCAT | |
| XPNPEP2 | AE116s1 | TGTAAAACGACGGCCAGTGGGCGCTCTGAGAAGA | 1021 | CAGGAAACAGCTATGACCACCCTGGGCAACAGAA | 1168 |
| | | CTAAT | TAAGA | | |
| XPNPEP2 | AE116s2 | TGTAAAACGACGGCCAGTGTCTTTTGTTTTGGAGG | 1022 | CAGGAAACAGCTATGACCGAAGGAGGAAAAAGG | 1169 |
| | | GCTC | | AGGAGC | |
| XPNPEP2 | AE116s4 | TGTAAAACGACGGCCAGTATGCACATACCACAGA | 1023 | CAGGAAACAGCTATGACCCCTCACACCCTATCCTA | 1170 |
| | | GGAGG | | CACG | |
| XPNPEP2 | AE116s3 | TGTAAAACGACGGCCAGTAACAGAAAAAGAGACT | 1024 | CAGGAAACAGCTATGACCGAGCCCTCCAAAACAA | 1171 |
| | | CGGGC | | AAGAC | |
| XPNPEP2 | AE116s29 | TGTAAAACGACGGCCAGTAATGATTGTCACCTGC | 1025 | CAGGAAACAGCTATGACCCTCCTTTGCAGAACAG | 1172 |
| | | AGACC | TCCAG | | |
| XPNPEP2 | AE116s30 | TGTAAAACGACGGCCAGTGAACCTAGTCCAGGTC | 1026 | CAGGAAACAGCTATGACCTGATTCAGGACACCTTT | 1173 |
| | | CCAAG | | CTGC | |
| XPNPEP2 | AE116s32 | TGTAAAACGACGGCCAGTATCCTGGCCTATTCATC | 1027 | CAGGAAACAGCTATGACCTATTGTCACCTGGCTCC | 1174 |
| | | CACT | | TCAC | |
| XPNPEP2 | AE116s33 | TGTAAAACGACGGCCAGTACTGAATCACCTGTGA | 1028 | CAGGAAACAGCTATGACCTGTTAGAGTAGGCTCA | 1175 |
| | | ATGCC | | GGGCA | |
| XPNPEP2 | AE116s7 | TGTAAAACGACGGCCAGTCTCTTCTGGCTCTTCCC | 1029 | CAGGAAACAGCTATGACCTTTGCAAACAAGAGTC | 1176 |
| | | AGTT | | GCTTT | |
| XPNPEP2 | AE116s18 | TGTAAAACGACGGCCAGTCCCCATCTAGATGGAG | 1030 | CAGGAAACAGCTATGACCGGACTATGGTGACAGC | 1177 |
| | | GGTTA | | TGGAG | |
| XPNPEP2 | AE116s28 | TGTAAAACGACGGCCAGTATTGCTCTCTTGGGGTT | 1031 | CAGGAAACAGCTATGACCGAGGCTCCAGACTCTC | 1178 |
| | | TTGT | | CTGTT | |
| XPNPEP2 | AE116s21 | TGTAAAACGACGGCCAGTCCACCTCACCCTCTCTT | 1032 | CAGGAAACAGCTATGACCCCTTCACCTTGTGTGGA | 1179 |
| | | CTTC | | CAGT | |
| GUCY1A2 | AE117s11 | TGTAAAACGACGGCCAGTGGAATTATTTGCCTCCT | 1033 | CAGGAAACAGCTATGACCGATGTTTTGCCGACAT | 1180 |
| | | CCAG | | GTTTT | |
| GUCY1A2 | AE117s13 | TGTAAAACGACGGCCAGTGACTGTGGGAAATGAC | 1034 | CAGGAAACAGCTATGACCACGTTATTGCCTGTTTG | 1181 |
| | | AACCT | | GAAA | |
| GUCY1A2 | AE117s7 | TGTAAAACGACGGCCAGTAACACCCATCACTTCA | 1035 | CAGGAAACAGCTATGACCTGAAAGCATAGACTGC | 1182 |
| | | CAAGG | | GTGTG | |
| GUCY1A2 | AE117s6 | TGTAAAACGACGGCCAGTAACACCCATCACTTCA | 1036 | CAGGAAACAGCTATGACCTGAAAGCATAGACTGC | 1183 |
| | | CAAGG | | GTGTG | |
| GUCY1A2 | AE117s10 | TGTAAAACGACGGCCAGTCACCTCGACTTTCCCTC | 1037 | CAGGAAACAGCTATGACCCAAATGCTGATGTGAA | 1184 |
| | | TCTT | TGGTG | | |
| GUCY1A2 | AE117s9 | TGTAAAACGACGGCCAGTCTTTGTTGGAGTGGTCT | 1038 | CAGGAAACAGCTATGACCGCCTTGATGAAGTGCT | 1185 |
| | | GCAT | | CTCAG | |
| GUCY1A2 | AE117s14 | TGTAAAACGACGGCCAGTACCAGTCCTTACCTCCA | 1039 | CAGGAAACAGCTATGACCACCATACTCAGCTTTG | 1186 |
| | | GGAA | | GGGTT | |
| GUCY1A2 | AE117s15 | TGTAAAACGACGGCCAGTTTGGGAAGTTTACCAC | 1040 | CAGGAAACAGCTATGACCGGTGACCGCTGTCAAT | 1187 |
| | | ACTGC | | AAAAA | |
| GUCY1A2 | AE117s1 | TGTAAAACGACGGCCAGTATAAGCTTTGGAATGA | 1041 | CAGGAAACAGCTATGACCCAAGACAGCACTGTTG | 1188 |
| | | AGCCG | CTGAG | | |
| GUCY1A2 | AE117s2 | TGTAAAACGACGGCCAGTCGAGTCAGTTCGAGAA | 1042 | CAGGAAACAGCTATGACCCAAGACAGCACTGTTG | 1189 |
| | | GCATT | CTGAG | | |
| GUCY1A2 | AE117s3 | TGTAAAACGACGGCCAGTGGTGCAAACAGATCGC | 1043 | CAGGAAACAGCTATGACCAAGGACAGGGGCAACA | 1190 |
| | | ATATT | TTTT | | |

TABLE VIII

| Status | Gender | Blacks | Caucasians |
|---|---|---|---|
| Cases | Females | 13 | 12 |
| | Males | 5 | 11 |
| | Total | 18 | 23 |
| Controls | Females | 5 | 0 |
| | Males | 2 | 0 |
| | Total | 7 | 0 |
| | ALL | 25 | 23 |

What is claimed is:

1. An isolated nucleic acid derived from a human gene encoding a protein selected from a member of the group consisting of: the C1, S subcomponent protein (C1S), the alanyl aminopeptidase protein (ANPEP), the meprin A, beta protein (MEP1B), the Aminopeptidase P-like protein (XPNPEPL), the tissue kallikrein protein (KLK1), the membrane bound aminopeptidase P protein (XPNPEP2), and the soluble guanylate cyclase 1, alpha-2 subunit protein (GUCY1A2), wherein said nucleic acid comprises at least one polymorphic position.

2. The isolated nucleic acid of claim 1 wherein said at least one polymorphic position for each said gene is a polymorphic position specified in a Table selected from the group consisting of: Table I, Table II, and Table III.

3. The isolated nucleic acid of claim 2 wherein the sequence at said at least one polymorphic position is depicted in a nucleic acid sequence selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182,183,184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, and 294, or complement thereof.

4. The isolated nucleic acid of claim 3 wherein said at least one polymorphic position resides in a non-coding position within the genomic sequence of said gene.

5. The isolated nucleic acid of claim 3 wherein said at least one polymorphic position resides in a coding position within the genomic sequence of said gene.

6. The isolated nucleic acid of claim 5 wherein said at least one polymorphic position residing in a coding position results in a missense mutation of the translated product of said gene.

7. The isolated nucleic acid of claim 5 wherein said at least one polymorphic position residing in a coding position results in a silent mutation of the translated product of said gene.

8. The isolated nucleic acid of claim 4 wherein said at least one polymorphic position residing in a non-coding position resides within the untranslated region of said gene.

9. The isolated nucleic acid of claim 4 wherein said at least one polymorphic position residing in a non-coding position resides within an intronic region of said gene.

10. A probe that hybridizes to a polymorphic position defined in claim 2.

11. The probe of claim 10 wherein said probe is at least 15 nucleotides in length.

12. The probe of claim 11 wherein a central position of the probe aligns with said polymorphic position.

13. The probe of claim 12 wherein the 3′ end of the primer aligns with said polymorphic position.

14. A method for genotyping an individual comprising the steps of
a. obtaining a nucleic acid sample(s) from said individual;
b. determining the nucleotide present at at least one polymorphic position of a polymorphic allele of claim 2, and
c. comparing said at least one polymorphic position with a known data set.

15. A method of constructing haplotypes using the isolated nucleic acids of claim 1, comprising the step of grouping at least two said nucleic acids.

16. The method according to claim 15 further comprising the step of using said haplotypes to identify an individual for the presence of a disease phenotype, and correlating the presence of the disease phenotype with said haplotype.

17. A method for identifying an individual at risk of developing a disorder upon administration of a pharmaceutically acceptable amount of an ACE inhibitor and/or vasopeptidase inhibitor comprising the steps of
a. amplifying one or more sequences from one or more nucleic acid samples using appropriate PCR primers for amplifying across at least one polymorphic position specified in claim 2;
b. comparing said at least one polymorphic position with a known data set; and
c. determining whether the result correlates with an increased or decreased risk for developing a disorder.

18. The method according to claim 17 wherein the disease phenotype is angioedema or an angioedema-like disorder.

19. A kit for identifying an individual at risk of developing a disorder upon administration of a pharmaceutically acceptable amount of an ACE inhibitor and/or vasopeptidase inhibitor, said kit comprising
a. sequencing primers, and
b. sequencing reagents, wherein said primers are primers that hybridize to at least one polymorphic position in a human gene selected from the group consisting of: the C1, S subcomponent protein (C1S), the alanyl aminopeptidase protein (ANPEP), the meprin A, beta protein (MEP1B), the Aminopeptidase P-like protein (XPNPEPL), the tissue kallikrein protein (KLK1), the membrane bound aminopeptidase P protein (XPNPEP2), and the soluble guanylate cyclase 1, alpha-2 subunit protein (GUCY1A2).
